(19) United States
(12) Patent Application Publication
(10) Pub. No.: US 2025/0146076 A1
(43) Pub. Date: May 8, 2025

(54) ALK GENE FUSIONS AND USES THEREOF

(71) Applicant: FOUNDATION MEDICINE, INC., Cambridge, MA (US)

(72) Inventors: Xin LIU, Boston, MA (US); Erica GORNSTEIN, Melrose, MA (US); Rachel ERBACH, Arlington, MA (US); Mark ROSENZWEIG, Norfolk, MA (US)

(73) Assignee: Foundation Medicine, Inc., Boston, MA (US)

(21) Appl. No.: 18/726,094

(22) PCT Filed: Jan. 13, 2023
(86) PCT No.: PCT/US2023/060663

§ 371 Date: Jul. 1, 2024

Related U.S. Application Data
(60) Provisional application No. 63/299,688, filed on Jan. 14, 2022.

(51) Int. Cl. C12Q 1/6886

(52) U.S. Cl. CPC C12Q 1/6886; C12Q 2600/156

(57) ABSTRACT

Provided herein are anaplastic lymphoma kinase (ALK) fusion nucleic acid molecules and polypeptides, methods related to detecting ALK fusion nucleic acid molecules and polypeptides in cancer, as well as methods of treatment and uses related thereto. Detection of an ALK fusion nucleic acid molecule or polypeptide can be used to identify individuals that may benefit from treatment with an anti-cancer therapy.

### CROSS-REFERENCE TO RELATED APPLICATIONS

This application claims the benefit of U.S. Provisional Application No. 63/299,688, filed Jan. 14, 2022, which is hereby incorporated by reference in its entirety.

### REFERENCE TO AN ELECTRONIC SEQUENCE LISTING

The contents of the electronic sequence listing (197102007340SEQLIST.xml; Size: 708,628 bytes; and Date of Creation: Jan. 12, 2023) are herein incorporated by reference in their entirety.

### TECHNICAL FIELD

Provided herein are anaplastic lymphoma kinase (ALK) fusion nucleic acid molecules and polypeptides, methods related to detecting such ALK fusion nucleic acid molecules and polypeptides, as well as methods of diagnosis/treatment and uses related thereto.

### BACKGROUND

Kinases activated by gene fusions are established oncogenic drivers and therapeutic targets, and have been associated with both hematopoietic malignancies and solid tumors. Kinase fusions have also been observed in patients following initial treatment with targeted therapies, suggesting that kinase fusions may be an acquired resistance mechanism, and that patients with such fusions could benefit from strategies that target the acquired kinase fusion. See, e.g, Xu et al., Cancer Manag Res (2019) 11:6343-51; Piotrowska et al., Cancer Discov (2018) 8(12):1529-39; Schrock et al., J Thorac Oncol (2018) 13(9):1312-23; and Schrock et al., J Thorac Oncol 2019; 14(2):255-64).

The anaplastic lymphoma kinase (ALK) gene encodes a receptor tyrosine kinase. ALK is a member of the insulin receptor superfamily. ALK activation induces downstream pathways associated with cell survival, angiogenesis, and cell proliferation (Grande et al., Molecular cancer therapeutics, vol. 10, 4 (2011): 569-79). ALK is a known oncogene that has been associated with cancerous phenotypes, including inflammatory myofibroblastic tumors, neuroblastoma, lung cancer, non-Hodgkin's lymphoma, and anaplastic large cell lymphoma, among others. Chromosomal rearrangements involving the ALK gene have been found in certain cancers, and have been characterized as oncogenic (see, e.g., Hallberg et al., Annals of oncology, vol. 27 Suppl 3 (2016): iii4-iii15; and Ignatius et al., JTO clinical and research reports, vol. 1, 1 100015, 2020). ALK gene fusions have also been associated with sensitivity of cancer to ALK inhibitors, such as crizotinib, ceritinib, alectinib, or entrectinib (see, e.g., Ali et al., USCAP Abstract 1868 2015; Mansfield et al., Annals of oncology, vol. 27, 11 (2016): 2111-2117; Yakirevich et al., Clinical cancer research, vol. 22, 15 (2016): 3831-40; Subbiah et al., Journal of hematology & oncology, vol. 8 66, 2015; Amatu et al., British journal of cancer vol. 113, 12 (2015): 1730-4; Lee et al., Oncotarget vol. 6, 27 (2015): 24320-32; Shan et al., Journal of thoracic oncology, vol. 10, 6 (2015): e37-9; Ali et al., The oncologist, vol. 21, 6 (2016): 762-70; and Ou et al., Journal of thoracic oncology, vol. 9, 12 (2014): 1821-5).

Thus, there is a need in the art for characterizing the cancer landscape of ALK kinase fusions, and for developing methods, compositions, and assays for evaluating and treating patients with such fusions.

All references cited herein, including patents, patent applications and publications, are hereby incorporated by reference in their entirety. To the extent that any reference incorporated by reference conflicts with the instant disclosure, the instant disclosure shall control.

### SUMMARY OF THE INVENTION

In one aspect, provided herein is a method of identifying an individual having a cancer who may benefit from a treatment comprising an anaplastic lymphoma kinase (ALK)-targeted therapy, the method comprising detecting in a sample from the individual: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule, wherein detection of the ALK fusion nucleic acid molecule or polypeptide in the sample identifies the individual as one who may benefit from the treatment comprising the ALK-targeted therapy.

In another aspect, provided herein is a method of selecting a therapy for an individual having a cancer, the method comprising detecting in a sample from the individual: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule, wherein detection of the ALK fusion nucleic acid molecule or polypeptide in the sample identifies the individual as one who may benefit from a treatment comprising an ALK-targeted therapy.

In another aspect, provided herein is a method of identifying one or more treatment options for an individual having a cancer, the method comprising: (a) detecting in a sample from the individual: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule; and (b) generating a report comprising one or more treatment options identified for the individual based at least in part on detection of the ALK fusion nucleic acid molecule or polypeptide in the sample, wherein the one or more treatment options comprise an ALK-targeted therapy.

In another aspect, provided herein is a method of identifying one or more treatment options for an individual having a cancer, the method comprising: (a) acquiring knowledge of: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule, in a sample from the individual; and (b) generating a report comprising one or more treatment options identified for the individual based at least in part on said knowledge, wherein the one or more treatment options comprise an ALK-targeted therapy.

In another aspect, provided herein is a method of selecting a treatment for an individual having cancer, comprising acquiring knowledge of: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule, in a sample from an individual having a cancer, wherein responsive to the acquisition of said knowledge: (i) the individual is classified as a candidate to receive a treatment comprising an ALK-targeted therapy; and/or (ii) the individual is identified as likely to respond to a treatment that comprises an ALK-targeted therapy.

In another aspect, provided herein is a method of predicting survival of an individual having a cancer, comprising acquiring knowledge of: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule, in a sample from the individual, wherein responsive to the acquisition of said knowledge, the individual is predicted to have longer survival when treated with a treatment comprising an ALK-targeted therapy, as compared to survival of an individual whose cancer does not comprise an ALK fusion nucleic acid molecule or polypeptide.

In another aspect, provided herein is a method of predicting survival of an individual having a cancer treated with a treatment comprising an ALK-targeted therapy, the method comprising acquiring knowledge of: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule, in a sample from the individual, wherein responsive to the acquisition of said knowledge, the individual is predicted to have longer survival when treated with a treatment comprising an ALK-targeted therapy, as compared to an individual whose cancer does not exhibit an ALK fusion nucleic acid molecule or polypeptide.

In another aspect, provided herein is a method of treating or delaying progression of cancer, comprising: (a) acquiring knowledge of: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule, in a sample from an individual having a cancer; and (b) responsive to said knowledge, administering to the individual an effective amount of a treatment that comprises an ALK-targeted therapy.

In another aspect, provided herein is a method of treating or delaying progression of cancer, comprising administering to an individual having cancer an effective amount of a treatment that comprises an ALK-targeted therapy, wherein the ALK-targeted therapy is administered responsive to acquiring knowledge of: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule, in a sample from the individual.

In another aspect, provided herein is a method of monitoring, evaluating or screening an individual having a cancer, comprising acquiring knowledge of: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule, in a sample from the individual, wherein responsive to the acquisition of said knowledge, the individual is predicted to have increased risk of cancer recurrence, aggressive cancer, anti-cancer therapy resistance, increased ALK expression, clinical benefit from an ALK-targeted therapy, or poor prognosis, as compared to an individual whose cancer does not comprise an ALK fusion nucleic acid molecule or polypeptide. In some embodiments, responsive to the acquisition of said knowledge, the individual is predicted to have resistance to a non-ALK-targeted anti-cancer therapy.

In another aspect, provided herein is a method of assessing an ALK fusion nucleic acid molecule or polypeptide in a cancer in an individual, the method comprising: (a) detecting in a sample from the individual: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule; and (b) providing an assessment of the ALK fusion nucleic acid molecule or polypeptide.

In another aspect, provided herein is a method of detecting an ALK fusion nucleic acid molecule or polypeptide, the method comprising detecting in a sample from an individual having a cancer: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule.

In another aspect, provided herein is a method of detecting the presence or absence of a cancer in an individual, the method comprising: (a) detecting the presence or absence of a cancer in a sample from the individual; and (b) detecting in a sample from the individual the presence or absence of: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule. In some embodiments, the method comprises detecting the presence of the cancer in a sample from the individual. In some embodiments, the method comprises detecting the presence of the ALK fusion nucleic acid molecule, or the ALK fusion polypeptide encoded by the ALK fusion nucleic acid molecule, in a sample from the individual.

In another aspect, provided herein is a method for monitoring progression or recurrence of a cancer in an individual, the method comprising: (a) detecting, in a first sample obtained from the individual at a first time point, the presence or absence of: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule; (b) detecting, in a second sample obtained from the individual at a second time point after the first time point, the presence or absence of: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule; and (c) providing an assessment of cancer progression or cancer recurrence in the individual based, at least in part, on the presence or absence of the ALK fusion nucleic acid molecule or the ALK fusion polypeptide in the first sample and/or in the second sample. In some embodiments, the presence of the ALK fusion nucleic acid molecule or the ALK fusion polypeptide in the first sample and/or in the second sample identifies the individual as having increased risk of cancer progression or cancer recurrence. In some embodiments, the method further comprises selecting a treatment, administering a treatment, adjusting a treatment, adjusting the dose of a treatment, or applying a treatment to the individual based, at least in part, on detecting the presence of the ALK fusion nucleic acid molecule or the ALK fusion polypeptide in the first sample and/or in the second sample, wherein the treatment comprises an ALK-targeted therapy.

In another aspect, provided herein is a method of detecting an ALK fusion nucleic acid molecule, the method comprising: (a) providing a plurality of nucleic acid molecules obtained from a sample from an individual having a cancer, wherein the plurality of nucleic acid molecules comprises nucleic acid molecules corresponding to an ALK fusion nucleic acid molecule comprising a fusion between an ALK gene, or a portion thereof, and a gene listed in Table 1, or a portion thereof; (b) optionally, ligating one or more adapters onto one or more nucleic acid molecules from the plurality of nucleic acid molecules; (c) optionally, amplifying the one or more ligated nucleic acid molecules from the plurality of nucleic acid molecules; (d) optionally, capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the ALK fusion nucleic acid molecule; (f) analyzing the plurality of sequence reads; and (g) based on the analysis, detecting the presence or absence of the ALK fusion nucleic acid molecule in the sample. In some embodiments, the method further comprises receiving, at one or more processors, sequence read data for the plurality of sequence reads. In some embodiments, the analyzing the plurality of sequence reads comprises identifying, using the one or more processors, the presence or absence of sequence reads corresponding to the ALK fusion nucleic acid molecule. In some embodiments, the amplified nucleic acid molecules are captured by hybridization with one or more bait molecules. In another aspect, provided herein is a method of detecting an ALK fusion nucleic acid molecule, the method comprising: (a) providing a sample from an individual having a cancer, wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to an ALK fusion nucleic acid molecule in said library to produce an enriched sample, wherein the ALK fusion nucleic acid molecule comprises a fusion between an ALK gene, or a portion thereof, and a gene listed in Table 1, or a portion thereof; (e) sequencing the enriched sample, thereby producing a plurality of sequence reads; (f) analyzing the plurality of sequence reads for the presence of the ALK fusion nucleic acid molecule; and (g) detecting, based on the analyzing step, the presence or absence of the ALK fusion nucleic acid molecule in the sample from the individual. In some embodiments, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample. In some embodiments, the one or more adapters comprise amplification primers, flow cell adapter sequences, substrate adapter sequences, sample index sequences, or unique molecular identifier (UMI) sequences. In some embodiments, the selectively enriching comprises: (a) combining one or more bait molecules with the library, thereby hybridizing the one or more bait molecules to one or more nucleic acid molecules comprising nucleotide sequences corresponding to the ALK fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer. In some embodiments, the method further comprises generating a molecular profile for the individual, based, at least in part, on detecting the presence or absence of the ALK fusion nucleic acid molecule. In some embodiments, the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile for the individual further comprises results from a nucleic acid sequencing-based test. In some embodiments, the method further comprises selecting a treatment, administering a treatment, or applying a treatment to the individual based on the generated molecular profile, wherein the treatment comprises an ALK-targeted therapy. In some embodiments, the method further comprises generating a report indicating the presence or absence of the ALK fusion nucleic acid molecule in the sample. In some embodiments, the method further comprises generating, by the one or more processors, a report indicating the presence or absence of the ALK fusion nucleic acid molecule in the sample. In some embodiments, the method further comprises transmitting the report to the individual, a caregiver, a healthcare provider, a physician, an oncologist, an electronic medical record system, a hospital, a clinic, a third-party payer, an insurance company, or a government office. In some embodiments, the report is transmitted via a computer network or a peer-to-peer connection.

In another aspect, provided herein is a method of identifying a candidate treatment for a cancer in an individual in need thereof, comprising performing DNA sequencing on a sample obtained from the individual to determine a sequencing mutation profile on a group of genes comprising one or more of ALK, one or more genes listed in Table 1, or any combination thereof, wherein the sequencing mutation profile identifies the presence or absence of an ALK fusion nucleic acid molecule, wherein the ALK fusion nucleic acid molecule comprises a fusion between an ALK gene, or a portion thereof, and a gene listed in Table 1, or a portion thereof. In some embodiments, the candidate treatment comprises an ALK-targeted therapy. In some embodiments, the presence of the ALK fusion nucleic acid molecule in the sample identifies the individual as one who may benefit from a treatment comprising an ALK-targeted therapy. In some embodiments, the presence of the ALK fusion nucleic acid molecule in the sample predicts the individual to have longer survival when treated with a treatment comprising an ALK-targeted therapy, as compared to survival of an individual whose cancer does not comprise an ALK fusion nucleic acid molecule. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencing mutation profile identifies the presence or absence of a fragment of the ALK fusion nucleic acid molecule comprising a breakpoint or fusion junction.

In another aspect, provided herein is a method of treating or delaying progression of cancer, comprising: (a) detecting in a sample from an individual having a cancer: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule; and (b) administering to the individual an effective amount of a treatment that comprises an ALK-targeted therapy.

In some embodiments, which may be combined with any of the preceding aspects or embodiments, the ALK fusion nucleic acid molecule is an ALK fusion nucleic acid molecule listed in Table 2, and wherein the order of the genes in the fusion in the 5′ to 3′ direction is as listed in Table 2. In some embodiments, which may be combined with any of the preceding aspects or embodiments, the ALK fusion nucleic acid molecule is an ALK fusion nucleic acid molecule listed in Table 3, and wherein the ALK fusion nucleic acid molecule comprises or results from a corresponding 5′ breakpoint and/or 3′ breakpoint within the exons or introns as listed in Table 3. In some embodiments, which may be combined with any of the preceding aspects or embodiments, the ALK fusion nucleic acid molecule is an ALK fusion nucleic acid molecule listed in Table 4, and wherein the ALK fusion nucleic acid molecule comprises or results from a corresponding 5′ breakpoint within the chromosomal coordinates as listed in Table 4, and/or a corresponding 3′ breakpoint within the chromosomal coordinates as listed in Table 4. In some embodiments, which may be combined with any of the preceding aspects or embodiments, the ALK fusion nucleic acid molecule is an ALK fusion nucleic acid molecule listed in Table 5, and wherein the ALK fusion nucleic acid molecule comprises or results from a fusion of a corresponding 5′ exon as listed in Table 5, or a portion thereof, fused to a corresponding 3′ exon as listed in Table 5, or a portion thereof. In some embodiments, which may be combined with any of the preceding aspects or embodiments, the ALK fusion nucleic acid molecule is an ALK fusion nucleic acid molecule listed in Table 6, and wherein the ALK fusion nucleic acid molecule comprises, in the 5′ to 3′ direction, the corresponding exons or portions thereof as listed in Table 6. In some embodiments, which may be combined with any of the preceding aspects or embodiments, the ALK fusion nucleic acid molecule is an ALK fusion nucleic acid molecule listed in Table 7, and wherein the ALK fusion nucleic acid molecule comprises a corresponding nucleotide sequence as listed in Table 7, or a nucleotide sequence with at least about 70% homology thereto. In some embodiments, which may be combined with any of the preceding aspects or embodiments, the ALK fusion nucleic acid molecule encodes an ALK fusion polypeptide. In some embodiments, which may be combined with any of the preceding aspects or embodiments, the ALK fusion nucleic acid molecule comprises a nucleotide sequence encoding an ALK fusion polypeptide that comprises an amino acid sequence as listed in Table 8, or an amino acid sequence with at least about 70% homology thereto. In some embodiments, which may be combined with any of the preceding aspects or embodiments, the ALK fusion polypeptide encoded by the ALK fusion nucleic acid molecule comprises an ALK kinase domain, or a fragment of an ALK kinase domain having ALK kinase activity. In some embodiments, which may be combined with any of the preceding aspects or embodiments, the ALK fusion polypeptide encoded by the ALK fusion nucleic acid molecule has ALK kinase activity. In some embodiments, which may be combined with any of the preceding aspects or embodiments, the ALK fusion polypeptide encoded by the ALK fusion nucleic acid molecule has a constitutive ALK kinase activity. In some embodiments, which may be combined with any of the preceding aspects or embodiments, the ALK fusion polypeptide encoded by the ALK fusion nucleic acid molecule is oncogenic. In some embodiments, which may be combined with any of the preceding aspects or embodiments, the ALK fusion polypeptide encoded by the ALK fusion nucleic acid molecule promotes cancer cell survival, angiogenesis, cancer cell proliferation, and any combination thereof. In some embodiments, which may be combined with any of the preceding aspects or embodiments, the ALK fusion polypeptide encoded by the ALK fusion nucleic acid molecule is capable of dimerizing with an ALK polypeptide or with another ALK fusion polypeptide in a cancer cell. In some embodiments, which may be combined with any of the preceding aspects or embodiments, the ALK fusion polypeptide encoded by the ALK fusion nucleic acid molecule is an ALK fusion polypeptide listed in Table 8, and wherein the ALK fusion polypeptide comprises a corresponding amino acid sequence as listed in Table 8, or an amino acid sequence with at least about 70% homology thereto.

In some embodiments, which may be combined with any of the preceding aspects or embodiments, the cancer is a carcinoma, a sarcoma, a lymphoma, a leukemia, a myeloma, a germ cell cancer, or a blastoma. In some embodiments, which may be combined with any of the preceding aspects or embodiments, the cancer is a solid tumor. In some embodiments, which may be combined with any of the preceding aspects or embodiments, the cancer is a hematologic malignancy. In some embodiments, which may be combined with any of the preceding aspects or embodiments, the cancer is a lymphoma. In some embodiments, which may be combined with any of the preceding aspects or embodiments, the cancer is a non-small cell lung carcinoma, a leiomyosarcoma, a thyroid carcinoma, a colorectal cancer, a pancreatic cancer, or a malignant peritoneal mesothelioma. In some embodiments, which may be combined with any of the preceding aspects or embodiments, the cancer is a B cell cancer, multiple myeloma, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, which may be combined with any of the preceding aspects or embodiments, the cancer is an anus squamous cell carcinoma, brain glioblastoma (GBM), breast cancer, breast carcinoma, breast invasive ductal carcinoma (IDC), colon adenocarcinoma (CRC), esophagus adenocarcinoma, fallopian tube serous carcinoma, gallbladder adenocarcinoma, gallbladder carcinoma, gastroesophageal junction adenocarcinoma, lung adenocarcinoma, lung non-small cell lung carcinoma, lung non-small cell lung carcinoma, lymph node Castleman's disease, lymph node lymphoma T-cell, ovary clear cell carcinoma, ovary endometrioid adenocarcinoma, ovary epithelial carcinoma, ovary high grade serous carcinoma, ovary serous carcinoma, pancreas cancer, pancreas ductal adenocarcinoma, pediatric bone osteosarcoma, bone osteosarcoma, pediatric skin melanoma, skin melanoma, pediatric soft tissue sarcoma, soft tissue sarcoma, pediatric soft tissue sarcoma undifferentiated, soft tissue sarcoma undifferentiated, peritoneum serous carcinoma, prostate acinar adenocarcinoma, small intestine adenocarcinoma, soft tissue leiomyosarcoma, soft tissue liposarcoma, thyroid papillary carcinoma, unknown primary adenocarcinoma, unknown primary carcinoma, unknown primary malignant neoplasm, unknown primary myoepithelial carcinoma, uterus carcinosarcoma, uterus endometrial adenocarcinoma endometrioid, uterus leiomyosarcoma, or vulva squamous cell carcinoma (SCC). In some embodiments, which may be combined with any of the preceding aspects or embodiments, the ALK fusion nucleic acid molecule is an ALK fusion nucleic acid molecule listed in Table 9, or the ALK fusion polypeptide is an ALK fusion polypeptide listed in Table 9, and wherein the cancer is the corresponding cancer as listed in Table 9. In some embodiments, which may be combined with any of the preceding aspects or embodiments, the cancer is metastatic. In some embodiments, the cancer has metastasized to the brain of the individual. In some embodiments, the individual has an intracranial metastasis of the cancer. In some embodiments, the individual has an extracranial metastasis of the cancer. In some embodiments, the cancer has not metastasized to the brain of the individual. In some embodiments, the individual does not have an intracranial metastasis of the cancer. In some embodiments, which may be combined with any of the preceding aspects or embodiments, the individual does not have an extracranial metastasis of the cancer.

In some embodiments, which may be combined with any of the preceding aspects or embodiments, the ALK-targeted therapy comprises one or more of a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof. In some embodiments, which may be combined with any of the preceding aspects or embodiments, the ALK-targeted therapy is a kinase inhibitor. In some embodiments, the ALK-targeted therapy is a tyrosine kinase inhibitor. In some embodiments, the ALK-targeted therapy is kinase inhibitor that inhibits the kinase activity of an ALK polypeptide. In some embodiments, the ALK-targeted therapy is a multi-kinase inhibitor or an ALK-specific inhibitor. In some embodiments, the ALK-targeted therapy comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A. In some embodiments, the nucleic acid inhibits the expression of the ALK fusion nucleic acid molecule or polypeptide. In some embodiments, the nucleic acid is a double-stranded RNA (dsRNA), a small interfering RNA (siRNA), or a small hairpin RNA (shRNA). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

In some embodiments, which may be combined with any of the preceding aspects or embodiments, the individual has received a prior anti-cancer treatment, or is being treated with an anti-cancer treatment. In some embodiments, the ALK fusion nucleic acid molecule, and/or the ALK fusion polypeptide encoded by the ALK fusion nucleic acid molecule, confers resistance of the cancer to the anti-cancer treatment. In some embodiments, the anti-cancer treatment is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, a chemotherapy, a targeted therapy, a non-ALK-targeted anti-cancer therapy, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, which may be combined with any of the preceding aspects or embodiments, the nucleic acid comprises a double-stranded RNA (dsRNA), a small interfering RNA (siRNA), or a small hairpin RNA (shRNA).

In some embodiments, which may be combined with any of the preceding aspects or embodiments, the cancer has not been previously treated. In some embodiments, which may be combined with any of the preceding aspects or embodiments, the ALK-targeted therapy is a first-line or front-line treatment. In some embodiments, the ALK-targeted therapy is brigatinib. In some embodiments, the ALK-targeted therapy is crizotinib. In some embodiments, the ALK-targeted therapy is lorlatinib. In some embodiments, the ALK-targeted therapy is a kinase inhibitor in combination with a PD-1- or a PD-L1-targeted agent. In some embodiments, the kinase inhibitor is a tyrosine kinase inhibitor. In some embodiments, the kinase inhibitor inhibits the kinase activity of an ALK polypeptide. In some embodiments, the kinase inhibitor is a multi-kinase inhibitor or an ALK-specific inhibitor. In some embodiments, the kinase inhibitor comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, and TAE684 (NVP-TAE684). In some embodiments, the PD-1-targeted agent is a PD-1 inhibitor. In some embodiments, the PD-1 inhibitor comprises one or more of nivolumab, pembrolizumab, cemiplimab, or dostarlimab. In some embodiments, the PD-L1-targeted agent is a PD-L1 inhibitor. In some embodiments, the PD-L1 inhibitor comprises one or more of atezolizumab, avelumab, or durvalumab. In some embodiments, the ALK-targeted therapy is alectinib in combination with a PD-1- or a PD-L1-targeted agent. In some embodiments, the ALK-targeted therapy is alectinib in combination with atezolizumab. In some embodiments, the cancer is a non-small cell lung carcinoma.

In some embodiments, which may be combined with any of the preceding aspects or embodiments, the cancer has not been previously treated with crizotinib. In some embodiments, which may be combined with any of the preceding aspects or embodiments, the cancer is crizotinib-naïve. In some embodiments, the ALK-targeted therapy is brigatinib.

In some embodiments, which may be combined with any of the preceding aspects or embodiments, the cancer is kinase inhibitor-naïve. In some embodiments, which may be combined with any of the preceding aspects or embodiments, the cancer has not been previously treated with a kinase inhibitor. In some embodiments, the ALK-targeted therapy is crizotinib or alectinib. In some embodiments, the ALK-targeted therapy is alectinib. In some embodiments, the ALK-targeted therapy is ceritinib. In some embodiments, the cancer is a non-small cell lung carcinoma. In some embodiments, the cancer is a kinase inhibitor-naïve non-small cell lung carcinoma.

In some embodiments, which may be combined with any of the preceding aspects or embodiments, the cancer has been previously treated with a kinase inhibitor. In some embodiments, which may be combined with any of the preceding aspects or embodiments, the cancer progressed on a prior treatment with a kinase inhibitor. In some embodiments, which may be combined with any of the preceding aspects or embodiments, the cancer is refractory to a prior kinase inhibitor treatment. In some embodiments, which may be combined with any of the preceding aspects or embodiments, the cancer progressed on a prior treatment with a chemotherapy and a kinase inhibitor. In some embodiments, the kinase inhibitor is a tyrosine kinase inhibitor. In some embodiments, the kinase inhibitor inhibits the kinase activity of an ALK polypeptide. In some embodiments, the kinase inhibitor is a multi-kinase inhibitor or an ALK-specific inhibitor. In some embodiments, the ALK-targeted therapy is lorlatinib. In some embodiments, the kinase inhibitor is crizotinib, and the ALK-targeted therapy is alectinib, ceritinib or brigatinib. In some embodiments, the kinase inhibitor is alectinib, and the ALK-targeted therapy is brigatinib. In some embodiments, the cancer progressed on a prior crizotinib treatment, and wherein the ALK-targeted therapy comprises continued treatment with crizotinib. In some embodiments, the cancer progressed on a prior lorlatinib treatment, and wherein the ALK-targeted therapy comprises continued treatment with lorlatinib. In some embodiments, the kinase inhibitor is crizotinib, and the ALK-targeted therapy is ceritinib. In some embodiments, the cancer is a non-small cell lung carcinoma.

In some embodiments, which may be combined with any of the preceding aspects or embodiments, the cancer is a solid tumor, and wherein the ALK-targeted therapy is entrectinib. In some embodiments, the cancer is a non-small cell lung carcinoma, a renal cell carcinoma, a colorectal cancer, or an inflammatory myofibroblastic tumor.

In some embodiments, which may be combined with any of the preceding aspects or embodiments, the ALK-targeted therapy is a fourth line treatment. In some embodiments, the ALK-targeted therapy is lorlatinib. In some embodiments, the cancer is an inflammatory myofibroblastic sarcoma, optionally, wherein the inflammatory myofibroblastic sarcoma is metastatic. In some embodiments, the individual has leptomeningeal disease.

In some embodiments, which may be combined with any of the preceding aspects or embodiments, the cancer further comprises one or more mutations in an ALK kinase domain encoded by an ALK gene. In some embodiments, which may be combined with any of the preceding aspects or embodiments, the ALK fusion nucleic acid molecule encodes an ALK fusion polypeptide comprising an ALK kinase domain, or a portion thereof, comprising one or more mutations in the ALK kinase domain. In some embodiments, which may be combined with any of the preceding aspects or embodiments, the ALK fusion polypeptide encoded by the ALK fusion nucleic acid molecule comprises an ALK kinase domain, or a portion thereof, comprising one or more mutations in the ALK kinase domain. In some embodiments, the one or more mutations in the ALK kinase domain comprise an amino acid substitution at amino acid position G1202 of the ALK kinase domain, optionally wherein the amino acid substitution is a G1202R amino acid substitution. In some embodiments, the ALK-targeted therapy is lorlatinib.

In some embodiments, which may be combined with any of the preceding aspects or embodiments, the cancer was previously treated with crizotinib, and wherein the ALK-targeted therapy is brigatinib. In some embodiments, the cancer is a non-small cell lung carcinoma.

In some embodiments, which may be combined with any of the preceding aspects or embodiments, the cancer is a colorectal cancer, and wherein the ALK-targeted therapy is ceritinib.

In some embodiments, which may be combined with any of the preceding aspects or embodiments, the cancer is a pancreatic cancer, and wherein the ALK-targeted therapy is ceritinib. In some embodiments, the cancer is a pancreatic ductal adenocarcinoma.

In some embodiments, which may be combined with any of the preceding aspects or embodiments, the ALK-targeted therapy is a kinase inhibitor in combination with a PD-1- or a PD-L1-targeted agent. In some embodiments, the kinase inhibitor is a tyrosine kinase inhibitor. In some embodiments, the kinase inhibitor inhibits the kinase activity of an ALK polypeptide. In some embodiments, the kinase inhibitor is a multi-kinase inhibitor or an ALK-specific inhibitor. In some embodiments, the kinase inhibitor comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, and TAE684 (NVP-TAE684). In some embodiments, the PD-1-targeted agent is a PD-1 inhibitor. In some embodiments, the PD-1 inhibitor comprises one or more of nivolumab, pembrolizumab, cemiplimab, or dostarlimab. In some embodiments, the PD-L1-targeted agent is a PD-L1 inhibitor. In some embodiments, the PD-L1 inhibitor comprises one or more of atezolizumab, avelumab, or durvalumab. In some embodiments, the ALK-targeted therapy is lorlatinib in combination with avelumab. In some embodiments, the cancer is a non-small cell lung carcinoma.

In some embodiments, which may be combined with any of the preceding aspects or embodiments, the treatment or the one or more treatment options further comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the additional anti-cancer therapy comprises one or more of a heat shock protein 90 inhibitor, an EGFR inhibitor, a SHP2 inhibitor, a MEK inhibitor, an IGF-1R inhibitor, a vascular endothelial growth factor (VEGF)-targeted therapy, or an mTOR inhibitor. In some embodiments, the nucleic acid comprises a double-stranded RNA (dsRNA), a small interfering RNA (siRNA), or a small hairpin RNA (shRNA).

In some embodiments, which may be combined with any of the preceding aspects or embodiments, the method further comprises obtaining the sample from the individual. In some embodiments, which may be combined with any of the preceding aspects or embodiments, the sample is obtained from the cancer. In some embodiments, which may be combined with any of the preceding aspects or embodiments, the sample comprises a tissue biopsy sample, a liquid biopsy sample, or a normal control. In some embodiments, which may be combined with any of the preceding aspects or embodiments, the sample is from a tumor biopsy, tumor specimen, or circulating tumor cell. In some embodiments, which may be combined with any of the preceding aspects or embodiments, the sample is a liquid biopsy sample and comprises blood, plasma, cerebrospinal fluid, sputum, stool, urine, or saliva. In some embodiments, which may be combined with any of the preceding aspects or embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, which may be combined with any of the preceding aspects or embodiments, the method comprises acquiring knowledge of or detecting the ALK fusion nucleic acid molecule or the ALK fusion polypeptide encoded by the ALK fusion nucleic acid molecule in a tissue biopsy sample, in a liquid biopsy sample, or in both a tissue biopsy sample and a liquid biopsy sample, from the individual.

In some embodiments, which may be combined with any of the preceding aspects or embodiments, the acquiring knowledge of the ALK fusion nucleic acid molecule or the ALK fusion polypeptide encoded by the ALK fusion nucleic acid molecule comprises detecting the ALK fusion nucleic acid molecule or polypeptide in the sample. In some embodiments, which may be combined with any of the preceding aspects or embodiments, detecting the ALK fusion nucleic acid molecule in the sample comprises detecting a fragment of the ALK fusion nucleic acid molecule comprising a breakpoint or fusion junction between the ALK gene, or the portion thereof, and the gene listed in Table 1, or the portion thereof. In some embodiments, which may be combined with any of the preceding aspects or embodiments, the ALK fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing. In some embodiments, the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS).

In some embodiments, which may be combined with any of the preceding aspects or embodiments, detecting the ALK fusion polypeptide comprises detecting a portion of the ALK fusion polypeptide that is encoded by a fragment of the ALK fusion nucleic acid molecule that comprises a breakpoint or fusion junction between the ALK gene, or the portion thereof, and the gene listed in Table 1, or the portion thereof. In some embodiments, which may be combined with any of the preceding aspects or embodiments, the ALK fusion polypeptide is detected in the sample by one or more of: immunoblotting, enzyme linked immunosorbent assay (ELISA), immunohistochemistry, or mass spectrometry.

In some embodiments, which may be combined with any of the preceding aspects or embodiments, the method further comprises selectively enriching for one or more nucleic acids in the sample comprising nucleotide sequences corresponding to the ALK fusion nucleic acid molecule; wherein the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the ALK fusion nucleic acid molecule and producing nucleic acid hybrids; and (b) isolating the nucleic acid hybrids to produce the enriched sample. In some embodiments, which may be combined with any of the preceding aspects or embodiments, the one or more bait molecules comprise a capture nucleic acid molecule configured to hybridize to a nucleotide sequence corresponding to the ALK fusion nucleic acid molecule. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, which may be combined with any of the preceding aspects or embodiments, the one or more bait molecules are conjugated to an affinity reagent or to a detection reagent. In some embodiments, the affinity reagent is an antibody, an antibody fragment, or biotin, or wherein the detection reagent is a fluorescent marker. In some embodiments, the capture nucleic acid molecule comprises a DNA, RNA, or mixed DNA/RNA molecule. In some embodiments, which may be combined with any of the preceding aspects or embodiments, the selectively enriching comprises amplifying the one or more nucleic acid molecules comprising nucleotide sequences corresponding to the ALK fusion nucleic acid molecule using a polymerase chain reaction (PCR) to produce an enriched sample. In some embodiments, which may be combined with any of the preceding aspects or embodiments, the method further comprises sequencing the enriched sample.

In some embodiments, which may be combined with any of the preceding aspects or embodiments, the method further comprises acquiring knowledge of or detecting in a sample from the individual a base substitution, a short insertion/deletion (indel), a copy number alteration, or a genomic rearrangement in one or more genes.

In some embodiments, which may be combined with any of the preceding aspects or embodiments, the ALK fusion nucleic acid molecule is an ALK fusion nucleic acid molecule listed in Table 2, and wherein the order of the genes in the fusion in the 5′ to 3′ direction is as listed in Table 2; the ALK fusion nucleic acid molecule is an ALK fusion nucleic acid molecule listed in Table 3, and wherein the ALK fusion nucleic acid molecule comprises or results from a corresponding 5′ breakpoint and/or 3′ breakpoint within the exons or introns as listed in Table 3; the ALK fusion nucleic acid molecule is an ALK fusion nucleic acid molecule listed in Table 4, and wherein the ALK fusion nucleic acid molecule comprises or results from a corresponding 5′ breakpoint within the chromosomal coordinates as listed in Table 4, and/or a corresponding 3′ breakpoint within the chromosomal coordinates as listed in Table 4; the ALK fusion nucleic acid molecule is an ALK fusion nucleic acid molecule listed in Table 5, and wherein the ALK fusion nucleic acid molecule comprises or results from a fusion of a corresponding 5′ exon as listed in Table 5, or a portion thereof, fused to a corresponding 3′ exon as listed in Table 5, or a portion thereof; the ALK fusion nucleic acid molecule is an ALK fusion nucleic acid molecule listed in Table 6, and wherein the ALK fusion nucleic acid molecule comprises, in the 5′ to 3′ direction, the corresponding exons or portions thereof as listed in Table 6; the ALK fusion nucleic acid molecule is an ALK fusion nucleic acid molecule listed in Table 7, and wherein the ALK fusion nucleic acid molecule comprises a corresponding nucleotide sequence as listed in Table 7, or a nucleotide sequence with at least about 70% homology thereto; and/or the ALK fusion nucleic acid molecule comprises a nucleotide sequence encoding an ALK fusion polypeptide that comprises an amino acid sequence as listed in Table 8, or an amino acid sequence with at least about 70% homology thereto.

In some embodiments, which may be combined with any of the preceding aspects or embodiments, the ALK fusion polypeptide encoded by the ALK fusion nucleic acid molecule: (a) comprises an ALK kinase domain, or a fragment of an ALK kinase domain having ALK kinase activity; (b) has ALK kinase activity; (c) has constitutive ALK kinase activity; (d) is oncogenic; (e) promotes cancer cell survival, angiogenesis, cancer cell proliferation, and any combination thereof; (f) is capable of dimerizing with an ALK polypeptide or with another ALK fusion polypeptide in a cancer cell; and/or (g) is an ALK fusion polypeptide listed in Table 8, and wherein the ALK fusion polypeptide comprises a corresponding amino acid sequence as listed in Table 8, or an amino acid sequence with at least about 70% homology thereto.

In some embodiments, which may be combined with any of the preceding aspects or embodiments, the cancer is: (a) a carcinoma, a sarcoma, a lymphoma, a leukemia, a myeloma, a germ cell cancer, or a blastoma; (b) a solid tumor; (c) a hematologic malignancy; (d) a lymphoma; (e) a non-small cell lung carcinoma, a leiomyosarcoma, a thyroid carcinoma, a colorectal cancer, a pancreatic cancer, or a malignant peritoneal mesothelioma; (f) a B cell cancer, multiple myeloma, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor; or (g) an anus squamous cell carcinoma, brain glioblastoma (GBM), breast cancer, breast carcinoma, breast invasive ductal carcinoma (IDC), colon adenocarcinoma (CRC), esophagus adenocarcinoma, fallopian tube serous carcinoma, gallbladder adenocarcinoma, gallbladder carcinoma, gastroesophageal junction adenocarcinoma, lung adenocarcinoma, lung non-small cell lung carcinoma, lung non-small cell lung carcinoma, lymph node Castleman's disease, lymph node lymphoma T-cell, ovary clear cell carcinoma, ovary endometrioid adenocarcinoma, ovary epithelial carcinoma, ovary high grade serous carcinoma, ovary serous carcinoma, pancreas cancer, pancreas ductal adenocarcinoma, pediatric bone osteosarcoma, bone osteosarcoma, pediatric skin melanoma, skin melanoma, pediatric soft tissue sarcoma, soft tissue sarcoma, pediatric soft tissue sarcoma undifferentiated, soft tissue sarcoma undifferentiated, peritoneum serous carcinoma, prostate acinar adenocarcinoma, small intestine adenocarcinoma, soft tissue leiomyosarcoma, soft tissue liposarcoma, thyroid papillary carcinoma, unknown primary adenocarcinoma, unknown primary carcinoma, unknown primary malignant neoplasm, unknown primary myoepithelial carcinoma, uterus carcinosarcoma, uterus endometrial adenocarcinoma endometrioid, uterus leiomyosarcoma, or vulva squamous cell carcinoma (SCC). In some embodiments, which may be combined with any of the preceding aspects or embodiments, the ALK fusion nucleic acid molecule is an ALK fusion nucleic acid molecule listed in Table 9 or the ALK fusion polypeptide is an ALK fusion polypeptide listed in Table 9, and wherein the cancer is the corresponding cancer as listed in Table 9. In some embodiments, which may be combined with any of the preceding aspects or embodiments, the cancer is metastatic; the cancer has metastasized to the brain of the individual; the individual has an intracranial metastasis of the cancer or an extracranial metastasis of the cancer; the cancer has not metastasized to the brain of the individual; the individual does not have an intracranial metastasis of the cancer; or the individual does not have an extracranial metastasis of the cancer.

In some embodiments, which may be combined with any of the preceding aspects or embodiments, the ALK-targeted therapy: (a) comprises one or more of a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof; (b) is a kinase inhibitor; (c) is a tyrosine kinase inhibitor, a kinase inhibitor that inhibits the kinase activity of an ALK polypeptide, a multi-kinase inhibitor, or an ALK-specific inhibitor; or (d) comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A.

In some embodiments, which may be combined with any of the preceding aspects or embodiments, the individual has received a prior anti-cancer treatment, or is being treated with an anti-cancer treatment; the cancer has not been previously treated; the cancer has not been previously treated with crizotinib; the cancer has not been previously treated with a kinase inhibitor; the cancer has been previously treated with a kinase inhibitor; or the cancer progressed on a prior treatment with a kinase inhibitor or is refractory to a prior kinase inhibitor treatment.

In some embodiments, which may be combined with any of the preceding aspects or embodiments, the ALK fusion nucleic acid molecule and/or the ALK fusion polypeptide encoded by the ALK fusion nucleic acid molecule confer resistance of the cancer to the prior anti-cancer treatment.

In some embodiments, which may be combined with any of the preceding aspects or embodiments, the cancer further comprises one or more mutations or rearrangements in an ALK kinase domain encoded by an ALK gene, and/or the ALK fusion nucleic acid molecule encodes an ALK fusion polypeptide comprising an ALK kinase domain, or a portion thereof, comprising one or more mutations in the ALK kinase domain.

In some embodiments, which may be combined with any of the preceding aspects or embodiments, the treatment further comprises an additional anti-cancer therapy. In some embodiments, the additional anti-cancer therapy comprises: (a) one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof; or (b) one or more of a heat shock protein 90 inhibitor, an EGFR inhibitor, a SHP2 inhibitor, a MEK inhibitor, an IGF-1R inhibitor, a vascular endothelial growth factor (VEGF)-targeted therapy, or an mTOR inhibitor.

In some embodiments, which may be combined with any of the preceding aspects or embodiments, the method further comprises obtaining the sample from the individual; the sample is obtained from the cancer; the sample comprises a tissue biopsy sample, a tumor biopsy sample, a tumor specimen, a liquid biopsy sample, a normal control, circulating tumor cells, circulating tumor DNA (ctDNA), cell-free DNA, or cell-free RNA; and/or the sample comprises cells and/or nucleic acids from the cancer.

In some embodiments, which may be combined with any of the preceding aspects or embodiments, the acquiring knowledge of the ALK fusion nucleic acid molecule or the ALK fusion polypeptide encoded by the ALK fusion nucleic acid molecule comprises detecting the ALK fusion nucleic acid molecule or polypeptide in the sample.

In some embodiments, which may be combined with any of the preceding aspects or embodiments, detecting the ALK fusion nucleic acid molecule in the sample comprises detecting a fragment of the ALK fusion nucleic acid molecule comprising a breakpoint or fusion junction between the ALK gene, or the portion thereof, and the gene listed in Table 1, or the portion thereof; and/or (b) detecting the ALK fusion polypeptide comprises detecting a portion of the ALK fusion polypeptide that is encoded by a fragment of the ALK fusion nucleic acid molecule that comprises a breakpoint or fusion junction between the ALK gene, or the portion thereof, and the gene listed in Table 1, or the portion thereof. In some embodiments, which may be combined with any of the preceding aspects or embodiments, the ALK fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing; and/or the ALK fusion polypeptide is detected in the sample by one or more of: immunoblotting, enzyme linked immunosorbent assay (ELISA), immunohistochemistry, or mass spectrometry.

In some embodiments, which may be combined with any of the preceding aspects or embodiments, the method further comprises acquiring knowledge of or detecting in a sample from the individual a base substitution, a short insertion/deletion (indel), a copy number alteration, or a genomic rearrangement in one or more genes.

In some embodiments, which may be combined with any of the preceding aspects or embodiments, the individual is a human.

In another aspect, provided herein is a kit comprising a probe or bait for detecting an ALK fusion nucleic acid molecule comprising a fusion between an ALK gene, or a portion thereof, and a gene listed in Table 1, or a portion thereof. In another aspect, provided herein is a kit comprising a probe or bait for detecting any of the ALK fusion nucleic acid molecules provided herein.

In another aspect, provided herein is a nucleic acid encoding an ALK fusion nucleic acid molecule, or a fragment thereof, comprising a fusion between an ALK gene, or a portion thereof, and a gene listed in Table 1, or a portion thereof. In another aspect, provided herein is a nucleic acid encoding any of the ALK fusion nucleic acid molecules provided herein, or a portion thereof.

In another aspect, provided herein is a vector comprising any of the nucleic acids provided herein. In another aspect, provided herein is a host cell comprising any of the vectors provided herein.

In another aspect, provided herein is an antibody or antibody fragment that specifically binds to an ALK fusion polypeptide, or to a portion thereof, wherein the ALK fusion polypeptide is encoded by an ALK fusion nucleic acid molecule comprising a fusion between an ALK gene, or a portion thereof, and a gene listed in Table 1, or a portion thereof. In another aspect, provided herein is an antibody or antibody fragment that specifically binds to any of the ALK fusion polypeptides provided herein, or to a portion thereof. In another aspect, provided herein is a kit comprising any of the antibodies or antibody fragments provided herein.

In another aspect, provided herein is an in vitro use of one or more oligonucleotides for detecting an ALK fusion nucleic acid molecule comprising a fusion between an ALK gene, or a portion thereof, and a gene listed in Table 1, or a portion thereof. In another aspect, provided herein is an in vitro use of one or more oligonucleotides for detecting any of the ALK fusion nucleic acid molecules provided herein, or a portion thereof. In another aspect, provided herein is a kit comprising one or more oligonucleotides for detecting an ALK fusion nucleic acid molecule comprising a fusion between an ALK gene, or a portion thereof, and a gene listed in Table 1, or a portion thereof. In another aspect, provided herein is a kit comprising one or more oligonucleotides for detecting any of the ALK fusion nucleic acid molecules provided herein, or a portion thereof.

In another aspect, provided herein is a system, comprising: a memory configured to store one or more program instructions; and one or more processors configured to execute the one or more program instructions, the one or more program instructions when executed by the one or more processors are configured to: (a) obtain a plurality of sequence reads of one or more nucleic acid molecules, wherein the one or more nucleic acid molecules are derived from a sample obtained from an individual; (b) analyze the plurality of sequence reads for the presence of an ALK fusion nucleic acid molecule comprising a fusion between an ALK gene, or a portion thereof, and a gene listed in Table 1, or a portion thereof; and (c) detect, based on the analyzing, the ALK fusion nucleic acid molecule in the sample.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acid molecules, wherein the one or more nucleic acid molecules are derived from a sample obtained from an individual; (b) analyzing, using the one or more processors, the plurality of sequence reads for the presence of an ALK fusion nucleic acid molecule comprising a fusion between an ALK gene, or a portion thereof, and a gene listed in Table 1, or a portion thereof; and (c) detecting, using the one or more processors and based on the analyzing, the ALK fusion nucleic acid molecule in the sample.

In some embodiments, which may be combined with any of the preceding aspects or embodiments, the sample is from an individual having a cancer. In some embodiments, which may be combined with any of the preceding aspects or embodiments, the plurality of sequence reads is obtained by sequencing, whole exome sequencing, whole genome sequencing, gene-targeted sequencing, or next-generation sequencing. In some embodiments, which may be combined with any of the preceding aspects or embodiments, the ALK fusion nucleic acid molecule is an ALK fusion nucleic acid molecule listed in Table 2, and wherein the order of the genes in the fusion in the 5′ to 3′ direction is as listed in Table 2. In some embodiments, which may be combined with any of the preceding aspects or embodiments, the ALK fusion nucleic acid molecule is an ALK fusion nucleic acid molecule listed in Table 3, and wherein the ALK fusion nucleic acid molecule comprises or results from a corresponding 5′ breakpoint and/or 3′ breakpoint within the exons or introns as listed in Table 3. In some embodiments, which may be combined with any of the preceding aspects or embodiments, the ALK fusion nucleic acid molecule is an ALK fusion nucleic acid molecule listed in Table 4, and wherein the ALK fusion nucleic acid molecule comprises or results from a corresponding 5′ breakpoint within the chromosomal coordinates as listed in Table 4, and/or a corresponding 3′ breakpoint within the chromosomal coordinates as listed in Table 4. In some embodiments, which may be combined with any of the preceding aspects or embodiments, the ALK fusion nucleic acid molecule is an ALK fusion nucleic acid molecule listed in Table 5, and wherein the ALK fusion nucleic acid molecule comprises or results from a fusion of a corresponding 5′ exon as listed in Table 5, or a portion thereof, fused to a corresponding 3′ exon as listed in Table 5, or a portion thereof. In some embodiments, which may be combined with any of the preceding aspects or embodiments, the ALK fusion nucleic acid molecule is an ALK fusion nucleic acid molecule listed in Table 6, and wherein the ALK fusion nucleic acid molecule comprises, in the 5′ to 3′ direction, the corresponding exons or portions thereof as listed in Table 6. In some embodiments, which may be combined with any of the preceding aspects or embodiments, the ALK fusion nucleic acid molecule is an ALK fusion nucleic acid molecule listed in Table 7, and wherein the ALK fusion nucleic acid molecule comprises a corresponding nucleotide sequence as listed in Table 7, or a nucleotide sequence with at least about 70% homology thereto. In some embodiments, which may be combined with any of the preceding aspects or embodiments, the ALK fusion nucleic acid molecule encodes an ALK fusion polypeptide. In some embodiments, which may be combined with any of the preceding aspects or embodiments, the ALK fusion nucleic acid molecule comprises a nucleotide sequence encoding an ALK fusion polypeptide that comprises an amino acid sequence as listed in Table 8, or an amino acid sequence with at least about 70% homology thereto. In some embodiments, which may be combined with any of the preceding aspects or embodiments, the encoded ALK fusion polypeptide comprises an ALK kinase domain, or a fragment of an ALK kinase domain, having ALK kinase activity. In some embodiments, which may be combined with any of the preceding aspects or embodiments, the encoded ALK fusion polypeptide has ALK kinase activity. In some embodiments, which may be combined with any of the preceding aspects or embodiments, the encoded ALK fusion polypeptide has a constitutive ALK kinase activity. In some embodiments, which may be combined with any of the preceding aspects or embodiments, the encoded ALK fusion polypeptide is oncogenic. In some embodiments, which may be combined with any of the preceding aspects or embodiments, the encoded ALK fusion polypeptide promotes cancer cell survival, angiogenesis, cancer cell proliferation, and any combination thereof. In some embodiments, which may be combined with any of the preceding aspects or embodiments, the encoded ALK fusion polypeptide is capable of dimerizing with an ALK polypeptide or with another ALK fusion polypeptide in a cancer cell. In some embodiments, which may be combined with any of the preceding aspects or embodiments, the encoded ALK fusion polypeptide is an ALK fusion polypeptide listed in Table 8, and wherein the encoded ALK fusion polypeptide comprises a corresponding amino acid sequence as listed in Table 8, or an amino acid sequence with at least about 70% homology thereto.

In some embodiments, which may be combined with any of the preceding aspects or embodiments, the cancer is a carcinoma, a sarcoma, a lymphoma, a leukemia, a myeloma, a germ cell cancer, or a blastoma. In some embodiments, which may be combined with any of the preceding aspects or embodiments, the cancer is a solid tumor. In some embodiments, which may be combined with any of the preceding aspects or embodiments, the cancer is a hematologic malignancy. In some embodiments, which may be combined with any of the preceding aspects or embodiments, the cancer is a lymphoma. In some embodiments, which may be combined with any of the preceding aspects or embodiments, the cancer is a non-small cell lung carcinoma, a leiomyosarcoma, a thyroid carcinoma, a colorectal cancer, a pancreatic cancer, or a malignant peritoneal mesothelioma. In some embodiments, which may be combined with any of the preceding aspects or embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, which may be combined with any of the preceding aspects or embodiments, the cancer is an anus squamous cell carcinoma, brain glioblastoma (GBM), breast cancer, breast carcinoma, breast invasive ductal carcinoma (IDC), colon adenocarcinoma (CRC), esophagus adenocarcinoma, fallopian tube serous carcinoma, gallbladder adenocarcinoma, gallbladder carcinoma, gastroesophageal junction adenocarcinoma, lung adenocarcinoma, lung non-small cell lung carcinoma, lung non-small cell lung carcinoma, lymph node Castleman's disease, lymph node lymphoma T-cell, ovary clear cell carcinoma, ovary endometrioid adenocarcinoma, ovary epithelial carcinoma, ovary high grade serous carcinoma, ovary serous carcinoma, pancreas cancer, pancreas ductal adenocarcinoma, pediatric bone osteosarcoma, bone osteosarcoma, pediatric skin melanoma, skin melanoma, pediatric soft tissue sarcoma, soft tissue sarcoma, pediatric soft tissue sarcoma undifferentiated, soft tissue sarcoma undifferentiated, peritoneum serous carcinoma, prostate acinar adenocarcinoma, small intestine adenocarcinoma, soft tissue leiomyosarcoma, soft tissue liposarcoma, thyroid papillary carcinoma, unknown primary adenocarcinoma, unknown primary carcinoma, unknown primary malignant neoplasm, unknown primary myoepithelial carcinoma, uterus carcinosarcoma, uterus endometrial adenocarcinoma endometrioid, uterus leiomyosarcoma, or vulva squamous cell carcinoma (SCC). In some embodiments, which may be combined with any of the preceding aspects or embodiments, the ALK fusion nucleic acid molecule is an ALK fusion nucleic acid molecule listed in Table 9, or the encoded ALK fusion polypeptide is an ALK fusion polypeptide listed in Table 9, and wherein the cancer is the corresponding cancer as listed in Table 9.

In some embodiments, which may be combined with any of the preceding aspects or embodiments, the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

In some embodiments, which may be combined with any of the preceding aspects or embodiments, the one or more program instructions when executed by the one or more processors are further configured to generate, based at least in part on the detecting, a molecular profile for the sample. In some embodiments, which may be combined with any of the preceding aspects or embodiments, the method further comprises generating, based at least in part on the detecting, a molecular profile for the sample. In some embodiments, the individual is administered a treatment based at least in part on the molecular profile. In some embodiments, the treatment comprises an ALK-targeted therapy. In some embodiments, the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test.

In another aspect, provided herein is an ALK-targeted therapy for use in a method of treating or delaying progression of cancer, wherein the method comprises administering the ALK-targeted therapy to an individual, wherein: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule, is detected in a sample obtained from the individual. In another aspect, provided herein is an ALK-targeted therapy for use in a method of treating or delaying progression of cancer, wherein the method comprises administering the ALK-targeted therapy to an individual, wherein: (a) an ALK fusion nucleic acid molecule provided herein, or a portion thereof, or (b) an ALK fusion polypeptide provided herein, or a portion thereof, is detected in a sample obtained from the individual.

In another aspect, provided herein is an ALK-targeted therapy for use in the manufacture of a medicament for treating or delaying progression of cancer, wherein the medicament is to be administered to an individual, wherein: (a) an ALK fusion nucleic acid molecule comprising a fusion between an ALK gene, or a portion thereof, and a gene listed in Table 1, or a portion thereof, or (b) an ALK fusion polypeptide encoded by the ALK fusion nucleic acid molecule, is detected in a sample obtained from the individual. In another aspect, provided herein is an ALK-targeted therapy for use in the manufacture of a medicament for treating or delaying progression of cancer, wherein the medicament is to be administered to an individual, wherein: (a) an ALK fusion nucleic acid molecule provided herein, or a portion thereof, or (b) an ALK fusion polypeptide provided herein, or a portion thereof, is detected in a sample obtained from the individual.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 depicts an exemplary device, in accordance with some embodiments.

FIG. 2 depicts an exemplary system, in accordance with some embodiments.

FIG. 3 depicts a block diagram of an exemplary process for detecting an ALK fusion nucleic acid molecule, in accordance with some embodiments.

### DETAILED DESCRIPTION

The present disclosure relates generally to detecting ALK kinase fusions in cancer, as well as methods of treatment, and uses related thereto.

Kinase fusions are an important class of targetable oncogenic driver variants. The present disclosure describes the results of comprehensive genomic profiling of the pan-cancer landscape of ALK gene fusions. These analyses identified diverse rearrangements leading to fusion genes involving ALK and numerous fusion partner genes (see, e.g., Example 1). Without wishing to be bound by theory, it is thought that the presence of an ALK fusion described herein in a sample from individuals having cancer may identify cancer patients who are likely to respond to treatment with an anti-cancer therapy such as a targeted anti-cancer therapy, e.g., an ALK-targeted therapy as described herein.

### I. General Techniques

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. Definitions

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

“Polynucleotide,” “nucleic acid,” or “nucleic acid molecule” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like), those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2-0-methyl-, 2-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(0)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“K”) and lambda (“X”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-1 13 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human lgG1 EU antibody.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-31 0 (2004); Lee et al., J. Mol. Biol. 340(5): 1073-1093 (2004); Fellouse, Proc. Natl. Acad. Sci. USA 101 (34): 12467-12472 (2004); and Lee et al., J. Immunol. Methods 284(1-2): 11 9-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and U.S. Pat. No. 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody.

A humanized antibody optionally may comprise at least a portion of an antibody constant region derived from a human antibody.

A “humanized form” of an antibody, e.g., a non-human antibody, refers to an antibody that has undergone humanization.

A “blocking” antibody or an “antagonist” antibody is one which inhibits or reduces biological activity of the antigen it binds. For example, blocking antibodies or antagonist antibodies substantially or completely inhibit the biological activity of the antigen.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

“Percent (%) amino acid sequence identity” or “homology” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, ALIGN-2, or Megalign (DNASTAR) software. Those skilled in the art can determine appropriate parameters for measuring alignment, including any algorithms needed to achieve maximal alignment over the full-length of the sequences being compared.

The term “detection” includes any means of detecting, including direct and indirect detection. The term “biomarker” as used herein (e.g., a “biomarker” such as a kinase fusion or a fusion nucleic acid molecule or polypeptide described herein) refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy including a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refer to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of an agent or a pharmaceutical composition (e.g., a pharmaceutical composition including the agent) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a reagent for specifically detecting a biomarker (e.g., a kinase fusion or a fusion nucleic acid molecule or polypeptide described herein) described herein. In certain embodiments, the manufacture or kit is promoted, distributed, or sold as a unit for performing the methods described herein.

The phrase “based on” when used herein means that the information about one or more biomarkers (e.g., a kinase fusion or a fusion nucleic acid molecule or polypeptide described herein) is used to inform a treatment decision, information provided on a package insert, or marketing/promotional guidance, etc.

### III. Methods, Systems, and Devices

In some aspects, provided herein are methods for identifying an individual having a cancer who may benefit from a treatment comprising an anaplastic lymphoma kinase (ALK)-targeted therapy. In other aspects, provided herein are methods for selecting a therapy or treatment for an individual having a cancer. In other aspects, provided herein are methods for identifying one or more treatment options for an individual having a cancer. In other aspects, provided herein are methods for predicting survival of an individual having a cancer. In other aspects, provided herein are methods for predicting survival of an individual having a cancer treated with a treatment comprising an ALK-targeted therapy. In other aspects, provided herein are methods for treating or delaying progression of cancer. In other aspects, provided herein are methods for monitoring, evaluating or screening an individual having a cancer. In other aspects, provided herein are methods for assessing an ALK fusion nucleic acid molecule or polypeptide in a cancer in an individual. In other aspects, provided herein are methods for detecting an ALK fusion nucleic acid molecule or polypeptide in a sample from an individual having a cancer. In other aspects, provided herein are methods for detecting the presence or absence of a cancer and/or an ALK fusion nucleic acid molecule or polypeptide in an individual.

In some embodiments of any of the methods provided herein, the methods comprise detecting the presence or absence of an ALK fusion nucleic acid molecule provided herein, or a fragment thereof, in a sample from an individual. In other embodiments of any of the methods provided herein, the methods comprise detecting the presence or absence of an ALK fusion polypeptide provided herein, or a fragment thereof, in a sample from an individual. In other embodiments of any of the methods provided herein, the methods comprise acquiring knowledge of the presence or absence of an ALK fusion polypeptide provided herein, or a fragment thereof, in a sample from an individual. In other embodiments of any of the methods provided herein, the methods comprise acquiring knowledge of the presence or absence of an ALK fusion nucleic acid molecule provided herein, or a fragment thereof, in a sample from an individual. In some embodiments, detection of an ALK fusion nucleic acid molecule or polypeptide of the disclosure, or a fragment thereof, in the sample identifies the individual as one who may benefit from the treatment comprising an ALK-targeted therapy. In some embodiments, the methods further comprise generating a report comprising one or more treatment options identified for the individual based at least in part on detection of the ALK fusion nucleic acid molecule or polypeptide, or a fragment thereof, in the sample, wherein the one or more treatment options comprise an ALK-targeted therapy. In some embodiments, the methods further comprise generating a report comprising one or more treatment options identified for the individual based at least in part on knowledge of the presence of the ALK fusion nucleic acid molecule or polypeptide, or a fragment thereof, in a sample from an individual, wherein the one or more treatment options comprise an ALK-targeted therapy. In some embodiments, responsive to the acquisition of knowledge of the presence of the ALK fusion nucleic acid molecule or polypeptide, or a fragment thereof, in a sample from an individual: (i) the individual is classified as a candidate to receive a treatment comprising an ALK-targeted therapy; and/or (ii) the individual is identified as likely to respond to a treatment that comprises an ALK-targeted therapy. In some embodiments, responsive to the acquisition of knowledge of the presence of the ALK fusion nucleic acid molecule or polypeptide, or a fragment thereof, in a sample from an individual, the individual is predicted to have longer survival when treated with a treatment comprising an ALK-targeted therapy, as compared to survival of an individual whose cancer does not comprise an ALK fusion nucleic acid molecule or polypeptide. In some embodiments, responsive to the acquisition of knowledge of the presence of the ALK fusion nucleic acid molecule or polypeptide, or a fragment thereof, in a sample from an individual, the method comprises administering to the individual an effective amount of a treatment that comprises an ALK-targeted therapy. In some embodiments, responsive to the acquisition of knowledge of the presence of the ALK fusion nucleic acid molecule or polypeptide, or a fragment thereof, in a sample from an individual, the individual is predicted to have increased risk of cancer recurrence, aggressive cancer, resistance to an anti-cancer therapy, e.g., a non-ALK-targeted therapy, poor prognosis, e.g., when treated with a non-ALK-targeted therapy, increased expression of ALK, or clinical benefit to ALK-targeted therapies, as compared to an individual whose cancer does not comprise an ALK fusion nucleic acid molecule or polypeptide. In some embodiments, the methods provided herein comprise providing an assessment of the ALK fusion nucleic acid molecule or polypeptide, or fragment thereof, e.g., in an individual or in a sample from an individual. In some embodiments, the methods provided herein comprise detecting the ALK fusion nucleic acid molecule or polypeptide, or a fragment thereof, in a sample from an individual, and administering to the individual an effective amount of a treatment that comprises an ALK-targeted therapy. In some embodiments, the methods provided herein comprise acquiring knowledge the presence of the ALK fusion nucleic acid molecule or polypeptide, or a fragment thereof, in a sample from an individual, and administering to the individual an effective amount of a treatment that comprises an ALK-targeted therapy.

In other aspects, provided herein are systems and non-transitory computer readable storage media. In some embodiments, a system of the disclosure comprises a memory configured to store one or more program instructions; and one or more processors configured to execute the one or more program instructions, the one or more program instructions when executed by the one or more processors are configured to: (a) obtain a plurality of sequence reads of one or more nucleic acids, wherein the one or more nucleic acids are derived from a sample obtained from an individual; (b) analyze the plurality of sequence reads for the presence of an ALK fusion nucleic acid molecule provided herein; and (c) detect, based on the analyzing, the ALK fusion nucleic acid molecule in the sample. In some embodiments, a non-transitory computer readable storage medium of the disclosure comprises one or more programs executable by one or more computer processors for performing a method, comprising: (a) obtaining, using the one or more processors, a plurality of sequence reads of one or more nucleic acids, wherein the one or more nucleic acids are derived from a sample obtained from an individual; (b) analyzing, using the one or more processors, the plurality of sequence reads for the presence of an ALK fusion nucleic acid molecule provided herein; and (c) detecting, using the one or more processors and based on the analyzing, the ALK fusion nucleic acid molecule in the sample.

### A. ALK Fusions

Certain aspects of the present disclosure relate to genomic rearrangements involving an anaplastic lymphoma kinase (ALK) gene, or a portion thereof. An ALK rearrangement of the present disclosure may relate to any chromosomal translocation, fusion, or rearrangement involving the locus of an ALK gene. In some embodiments, the rearrangements of the disclosure result in an ALK fusion nucleic acid molecule that comprises at least a portion of an ALK gene fused to at least a portion of another gene, such as any of an ABCB11, ACTN4, AGAP1, APH1A, AZI2, BTBD9, C2orf73, CAPN14, CARMIL1, CASP8, CDC42BPA, CIB4, CNTNAP5, COL3A1, CPQ, CPSF7, CREBBP, CTBP1, CTNND1, CYP51A1, CYS1, EPHA2, FHOD3, FILIP1L, GMCL1, GPN1, GPR113, HADHA, HS1BP3, INTS9, ITGA6, KCTD18, KIF5C, KLC4, LINC00535, LRRFIP2, MAGOHB, MAMDC4, MANBA, MAP3K9, MED13L, METTL25, MTBP, MYH10, MYO5C, NFIA, NINJ2, OPRM1, OTX1, PAQR4, PDCD10, PDE3A, PELI1, PLEC, PTGER4, PTPRJ, QKI, RPS6KA5, SASH1, SEC16B, SKAP1, SLC25A13, SLC30A6, SNX17, SOX13, SRSF7, TANGO6, TG, TMCO3, TNS3, TRIM24, TTC28, UBE2L3, UBE3B, UTRN, VASP, WDR92, YPEL5, ZNF446, ZNF454, ZNF513, or ZSWIM2 gene, or a gene listed in Table 1. Accordingly, certain aspects of the present disclosure relate to ALK fusion nucleic acid molecules, as well as to ALK fusion polypeptides encoded by such ALK fusion nucleic acid molecules.

As used herein “anaplastic lymphoma kinase” or “ALK” refer to a gene encoding an ALK mRNA or polypeptide. The ALK gene encodes the ALK receptor tyrosine kinase protein. ALK is also known as CD246, NBLST3, anaplastic lymphoma receptor tyrosine kinase, and ALK receptor tyrosine kinase. In some embodiments, an ALK gene is a human ALK gene. An exemplary ALK gene is represented by NCBI Gene ID No. 238. Exemplary ALK transcript sequences are represented by NCBI Ref. Seq. NM_004304 and provided below as SEQ ID NO: 90. Exemplary amino acid sequences of an ALK polypeptide are represented by NCBI Ref. Seq. NP_004295 and provided below as SEQ ID NO: 185.

(SEQ ID NO: 90)
ATGGGAGCCATCGGGCTCCTGTGGCTCCTGCCGCTGCTGCTTTCCACGGCAGCTGTGGGCTCCGGGATGGGGACC
GGCCAGCGCGCGGGCTCCCCAGCTGCGGGGCCGCCGCTGCAGCCCCGGGAGCCACTCAGCTACTCGCGCCTGCAG
AGGAAGAGTCTGGCAGTTGACTTCGTGGTGCCCTCGCTCTTCCGTGTCTACGCCCGGGACCTACTGCTGCCACCA
TCCTCCTCGGAGCTGAAGGCTGGCAGGCCCGAGGCCCGCGGCTCGCTAGCTCTGGACTGCGCCCCGCTGCTCAGG
TTGCTGGGGCCGGCGCCGGGGGTCTCCTGGACCGCCGGTTCACCAGCCCCGGCAGAGGCCCGGACGCTGTCCAGG
GTGCTGAAGGGCGGCTCCGTGCGCAAGCTCCGGCGTGCCAAGCAGTTGGTGCTGGAGCTGGGCGAGGAGGCGATC
TTGGAGGGTTGCGTCGGGCCCCCCGGGGAGGCGGCTGTGGGGCTGCTCCAGTTCAATCTCAGCGAGCTGTTCAGT
TGGTGGATTCGCCAAGGCGAAGGGCGACTGAGGATCCGCCTGATGCCCGAGAAGAAGGCGTCGGAAGTGGGCAGA
GAGGGAAGGCTGTCCGCGGCAATTCGCGCCTCCCAGCCCCGCCTTCTCTTCCAGATCTTCGGGACTGGTCATAGC
TCCTTGGAATCACCAACAAACATGCCTTCTCCTTCTCCTGATTATTTTACATGGAATCTCACCTGGATAATGAAA
GACTCCTTCCCTTTCCTGTCTCATCGCAGCCGATATGGTCTGGAGTGCAGCTTTGACTTCCCCTGTGAGCTGGAG
TATTCCCCTCCACTGCATGACCTCAGGAACCAGAGCTGGTCCTGGCGCCGCATCCCCTCCGAGGAGGCCTCCCAG
ATGGACTTGCTGGATGGGCCTGGGGCAGAGCGTTCTAAGGAGATGCCCAGAGGCTCCTTTCTCCTTCTCAACACC
TCAGCTGACTCCAAGCACACCATCCTGAGTCCGTGGATGAGGAGCAGCAGTGAGCACTGCACACTGGCCGTCTCG
GTGCACAGGCACCTGCAGCCCTCTGGAAGGTACATTGCCCAGCTGCTGCCCCACAACGAGGCTGCAAGAGAGATC
CTCCTGATGCCCACTCCAGGGAAGCATGGTTGGACAGTGCTCCAGGGAAGAATCGGGCGTCCAGACAACCCATTT
CGAGTGGCCCTGGAATACATCTCCAGTGGAAACCGCAGCTTGTCTGCAGTGGACTTCTTTGCCCTGAAGAACTGC
AGTGAAGGAACATCCCCAGGCTCCAAGATGGCCCTGCAGAGCTCCTTCACTTGTTGGAATGGGACAGTCCTCCAG
CTTGGGCAGGCCTGTGACTTCCACCAGGACTGTGCCCAGGGAGAAGATGAGAGCCAGATGTGCCGGAAACTGCCT
GTGGGTTTTTACTGCAACTTTGAAGATGGCTTCTGTGGCTGGACCCAAGGCACACTGTCACCCCACACTCCTCAA
TGGCAGGTCAGGACCCTAAAGGATGCCCGGTTCCAGGACCACCAAGACCATGCTCTATTGCTCAGTACCACTGAT
GTCCCCGCTTCTGAAAGTGCTACAGTGACCAGTGCTACGTTTCCTGCACCGATCAAGAGCTCTCCATGTGAGCTC
CGAATGTCCTGGCTCATTCGTGGAGTCTTGAGGGGAAACGTGTCCTTGGTGCTAGTGGAGAACAAAACCGGGAAG
GAGCAAGGCAGGATGGTCTGGCATGTCGCCGCCTATGAAGGCTTGAGCCTGTGGCAGTGGATGGTGTTGCCTCTC
CTCGATGTGTCTGACAGGTTCTGGCTGCAGATGGTCGCATGGTGGGGACAAGGATCCAGAGCCATCGTGGCTTTT
GACAATATCTCCATCAGCCTGGACTGCTACCTCACCATTAGCGGAGAGGACAAGATCCTGCAGAATACAGCACCC
AAATCAAGAAACCTGTTTGAGAGAAACCCAAACAAGGAGCTGAAACCCGGGGAAAATTCACCAAGACAGACCCCC
ATCTTTGACCCTACAGTTCATTGGCTGTTCACCACATGTGGGGCCAGCGGGCCCCATGGCCCCACCCAGGCACAG
TGCAACAACGCCTACCAGAACTCCAACCTGAGCGTGGAGGTGGGGAGCGAGGGCCCCCTGAAAGGCATCCAGATC
TGGAAGGTGCCAGCCACCGACACCTACAGCATCTCGGGCTACGGAGCTGCTGGCGGGAAAGGCGGGAAGAACACC
ATGATGCGGTCCCACGGCGTGTCTGTGCTGGGCATCTTCAACCTGGAGAAGGATGACATGCTGTACATCCTGGTT
GGGCAGCAGGGAGAGGACGCCTGCCCCAGTACAAACCAGTTAATCCAGAAAGTCTGCATTGGAGAGAACAATGTG
ATAGAAGAAGAAATCCGTGTGAACAGAAGCGTGCATGAGTGGGCAGGAGGCGGAGGAGGAGGGGGTGGAGCCACC
TACGTATTTAAGATGAAGGATGGAGTGCCGGTGCCCCTGATCATTGCAGCCGGAGGTGGTGGCAGGGCCTACGGG
GCCAAGACAGACACGTTCCACCCAGAGAGACTGGAGAATAACTCCTCGGTTCTAGGGCTAAACGGCAATTCCGGA
GCCGCAGGTGGTGGAGGTGGCTGGAATGATAACACTTCCTTGCTCTGGGCCGGAAAATCTTTGCAGGAGGGTGCC
ACCGGAGGACATTCCTGCCCCCAGGCCATGAAGAAGTGGGGGTGGGAGACAAGAGGGGGTTTCGGAGGGGGTGGA
GGGGGGTGCTCCTCAGGTGGAGGAGGCGGAGGATATATAGGCGGCAATGCAGCCTCAAACAATGACCCCGAAATG
GATGGGGAAGATGGGGTTTCCTTCATCAGTCCACTGGGCATCCTGTACACCCCAGCTTTAAAAGTGATGGAAGGC
CACGGGGAAGTGAATATTAAGCATTATCTAAACTGCAGTCACTGTGAGGTAGACGAATGTCACATGGACCCTGAA
AGCCACAAGGTCATCTGCTTCTGTGACCACGGGACGGTGCTGGCTGAGGATGGCGTCTCCTGCATTGTGTCACCC
ACCCCGGAGCCACACCTGCCACTCTCGCTGATCCTCTCTGTGGTGACCTCTGCCCTCGTGGCCGCCCTGGTCCTG
GCTTTCTCCGGCATCATGATTGTGTACCGCCGGAAGCACCAGGAGCTGCAAGCCATGCAGATGGAGCTGCAGAGC
CCTGAGTACAAGCTGAGCAAGCTCCGCACCTCGACCATCATGACCGACTACAACCCCAACTACTGCTTTGCTGGC
AAGACCTCCTCCATCAGTGACCTGAAGGAGGTGCCGCGGAAAAACATCACCCTCATTCGGGGTCTGGGCCATGGC
GCCTTTGGGGAGGTGTATGAAGGCCAGGTGTCCGGAATGCCCAACGACCCAAGCCCCCTGCAAGTGGCTGTGAAG
ACGCTGCCTGAAGTGTGCTCTGAACAGGACGAACTGGATTTCCTCATGGAAGCCCTGATCATCAGCAAATTCAAC
CACCAGAACATTGTTCGCTGCATTGGGGTGAGCCTGCAATCCCTGCCCCGGTTCATCCTGCTGGAGCTCATGGCG
GGGGGAGACCTCAAGTCCTTCCTCCGAGAGACCCGCCCTCGCCCGAGCCAGCCCTCCTCCCTGGCCATGCTGGAC
CTTCTGCACGTGGCTCGGGACATTGCCTGTGGCTGTCAGTATTTGGAGGAAAACCACTTCATCCACCGAGACATT
GCTGCCAGAAACTGCCTCTTGACCTGTCCAGGCCCTGGAAGAGTGGCCAAGATTGGAGACTTCGGGATGGCCCGA
GACATCTACAGGGCGAGCTACTATAGAAAGGGAGGCTGTGCCATGCTGCCAGTTAAGTGGATGCCCCCAGAGGCC
TTCATGGAAGGAATATTCACTTCTAAAACAGACACATGGTCCTTTGGAGTGCTGCTATGGGAAATCTTTTCTCTT
GGATATATGCCATACCCCAGCAAAAGCAACCAGGAAGTTCTGGAGTTTGTCACCAGTGGAGGCCGGATGGACCCA
CCCAAGAACTGCCCTGGGCCTGTATACCGGATAATGACTCAGTGCTGGCAACATCAGCCTGAAGACAGGCCCAAC
TTTGCCATCATTTTGGAGAGGATTGAATACTGCACCCAGGACCCGGATGTAATCAACACCGCTTTGCCGATAGAA
TATGGTCCACTTGTGGAAGAGGAAGAGAAAGTGCCTGTGAGGCCCAAGGACCCTGAGGGGGTTCCTCCTCTCCTG
GTCTCTCAACAGGCAAAACGGGAGGAGGAGCGCAGCCCAGCTGCCCCACCACCTCTGCCTACCACCTCCTCTGGC
AAGGCTGCAAAGAAACCCACAGCTGCAGAGATCTCTGTTCGAGTCCCTAGAGGGCCGGCCGTGGAAGGGGGACAC
GTGAATATGGCATTCTCTCAGTCCAACCCTCCTTCGGAGTTGCACAAGGTCCACGGATCCAGAAACAAGCCCACC
AGCTTGTGGAACCCAACGTACGGCTCCTGGTTTACAGAGAAACCCACCAAAAAGAATAATCCTATAGCAAAGAAG
GAGCCACACGACAGGGGTAACCTGGGGCTGGAGGGAAGCTGTACTGTCCCACCTAACGTTGCAACTGGGAGACTT
CCGGGGGCCTCACTGCTCCTAGAGCCCTCTTCGCTGACTGCCAATATGAAGGAGGTACCTCTGTTCAGGCTACGT
CACTTCCCTTGTGGGAATGTCAATTACGGCTACCAGCAACAGGGCTTGCCCTTAGAAGCCGCTACTGCCCCTGGA
GCTGGTCATTACGAGGATACCATTCTGAAAAGCAAGAATAGCATGAACCAGCCTGGGCCCTGA

Exemplary amino acid sequences of an ALK polypeptide are represented by NCBI Ref. Seq. NP_004295 and provided below as SEQ ID NO: 185.

(SEQ ID NO: 185)
MGAIGLLWLLPLLLSTAAVGSGMGTGQRAGSPAAGPPLQPREPLSYSRLQRKSLAVDFVVPSLFRVYARDLLLPP
SSSELKAGRPEARGSLALDCAPLLRLLGPAPGVSWTAGSPAPAEARTLSRVLKGGSVRKLRRAKQLVLELGEEAI
LEGCVGPPGEAAVGLLQFNLSELFSWWIRQGEGRLRIRLMPEKKASEVGREGRLSAAIRASQPRLLFQIFGTGHS
SLESPTNMPSPSPDYFTWNLTWIMKDSFPFLSHRSRYGLECSFDFPCELEYSPPLHDLRNQSWSWRRIPSEEASQ
MDLLDGPGAERSKEMPRGSFLLLNTSADSKHTILSPWMRSSSEHCTLAVSVHRHLQPSGRYIAQLLPHNEAAREI
LLMPTPGKHGWTVLQGRIGRPDNPFRVALEYISSGNRSLSAVDFFALKNCSEGTSPGSKMALQSSFTCWNGTVLQ
LGQACDFHQDCAQGEDESQMCRKLPVGFYCNFEDGFCGWTQGTLSPHTPQWQVRTLKDARFQDHQDHALLLSTTD
VPASESATVTSATFPAPIKSSPCELRMSWLIRGVLRGNVSLVLVENKTGKEQGRMVWHVAAYEGLSLWQWMVLPL
LDVSDREWLQMVAWWGQGSRAIVAFDNISISLDCYLTISGEDKILQNTAPKSRNLFERNPNKELKPGENSPRQTP
IFDPTVHWLFTTCGASGPHGPTQAQCNNAYQNSNLSVEVGSEGPLKGIQIWKVPATDTYSISGYGAAGGKGGKNT
MMRSHGVSVLGIFNLEKDDMLYILVGQQGEDACPSTNQLIQKVCIGENNVIEEEIRVNRSVHEWAGGGGGGGGAT
YVFKMKDGVPVPLIIAAGGGGRAYGAKTDTFHPERLENNSSVLGLNGNSGAAGGGGGWNDNISLLWAGKSLQEGA
TGGHSCPQAMKKWGWETRGGFGGGGGGCSSGGGGGGYIGGNAASNNDPEMDGEDGVSFISPLGILYTPALKVMEG
HGEVNIKHYLNCSHCEVDECHMDPESHKVICFCDHGTVLAEDGVSCIVSPTPEPHLPLSLILSVVTSALVAALVL
AFSGIMIVYRRKHQELQAMQMELQSPEYKLSKLRTSTIMTDYNPNYCFAGKTSSISDLKEVPRKNITLIRGLGHG
AFGEVYEGQVSGMPNDPSPLQVAVKTLPEVCSEQDELDFLMEALIISKFNHQNIVRCIGVSLQSLPRFILLELMA
GGDLKSFLRETRPRPSQPSSLAMLDLLHVARDIACGCQYLEENHFIHRDIAARNCLLTCPGPGRVAKIGDFGMAR
DIYRASYYRKGGCAMLPVKWMPPEAFMEGIFTSKTDTWSFGVLLWEIFSLGYMPYPSKSNQEVLEFVTSGGRMDP
PKNCPGPVYRIMTQCWQHQPEDRPNFAIILERIEYCTQDPDVINTALPIEYGPLVEEEEKVPVRPKDPEGVPPLL
VSQQAKREEERSPAAPPPLPTTSSGKAAKKPTAAEISVRVPRGPAVEGGHVNMAFSQSNPPSELHKVHGSRNKPT
SLWNPTYGSWFTEKPTKKNNPIAKKEPHDRGNLGLEGSCTVPPNVATGRLPGASLLLEPSSLTANMKEVPLERLR
HFPCGNVNYGYQQQGLPLEAATAPGAGHYEDTILKSKNSMNQPGP

As used herein “ABCB11” refers to a gene encoding an ABCB11 mRNA or polypeptide. The ABCB11 gene encodes a bile salt export pump protein. ABCB11 is also known as ABC16, BRIC2, BSEP, PFIC-2, PFIC2, PGY4, and SPGP. In some embodiments, an ABCB11 gene is a human ABCB11 gene. An exemplary ABCB11 gene is represented by NCBI Gene ID No. 8647. Exemplary ABCB11 transcript sequences are represented by NCBI Ref. Seq. NM_003742 and provided herein as SEQ ID NO: 87. Exemplary amino acid sequences of an ABCB11 polypeptide are represented by NCBI Ref. Seq. NP_003733 and provided herein as SEQ ID NO: 182.

As used herein “ACTN4” refers to a gene encoding an ACTN4 mRNA or polypeptide. The ACTN4 gene encodes the alpha-actinin-4 protein. ACTN4 is also known as ACTININ-4, FSGS, and FSGS1. In some embodiments, an ACTN4 gene is a human ACTN4 gene. An exemplary ACTN4 gene is represented by NCBI Gene ID No. 81. Exemplary ACTN4 transcript sequences are represented by NCBI Ref. Seq. NM_004924 and provided herein as SEQ ID NO: 88. Exemplary amino acid sequences of an ACTN4 polypeptide are represented by NCBI Ref. Seq. NP_004915 and provided herein as SEQ ID NO: 183.

As used herein “AGAP1” refers to a gene encoding an AGAP1 mRNA or polypeptide. The AGAP1 gene encodes an arf-GAP with GTPase, ANK repeat and PH domain-containing protein 1 protein. AGAP1 is also known as AGAP-1, CENTG2, cnt-g2, and GGAP1. In some embodiments, an AGAP1 gene is a human AGAP1 gene. An exemplary AGAP1 gene is represented by NCBI Gene ID No. 116987. Exemplary AGAP1 transcript sequences are represented by NCBI Ref. Seq. NM_014914 and provided herein as SEQ ID NO: 89. Exemplary amino acid sequences of an AGAP1 polypeptide are represented by NCBI Ref. Seq. NP_055729 and provided herein as SEQ ID NO: 184.

As used herein “APH1A” refers to a gene encoding an APH1A mRNA or polypeptide. The APH1A gene encodes a gamma-secretase subunit APH-1A protein. APH1A is also known as 6530402N02Rik, APH-1, APH-1A, and CGI-78. In some embodiments, an APH1A gene is a human APH1A gene. An exemplary APH1A gene is represented by NCBI Gene ID No. 51107. Exemplary APH1A transcript sequences are represented by NCBI Ref. Seq. NM_016022 and provided herein as SEQ ID NO: 91. Exemplary amino acid sequences of an APH1A polypeptide are represented by NCBI Ref. Seq. NP_057106.2 and provided herein as SEQ ID NO: 186.

As used herein “AZI2” refers to a gene encoding an AZI2 mRNA or polypeptide. The AZI2 gene encodes a 5-azacytidine-induced protein 2 protein. AZI2 is also known as AZ2, NAP1, and TILP. In some embodiments, an AZI2 gene is a human AZI2 gene. An exemplary AZI2 gene is represented by NCBI Gene ID No. 64343. Exemplary AZI2 transcript sequences are represented by NCBI Ref. Seq. NM_022461 and provided herein as SEQ ID NO: 92. Exemplary amino acid sequences of an AZI2 polypeptide are represented by NCBI Ref. Seq. NP_071906 and provided herein as SEQ ID NO: 187.

As used herein “BTBD9” refers to a gene encoding a BTBD9 mRNA or polypeptide. The BTBD9 gene encodes the BTB/POZ domain-containing protein 9. BTBD9 is also known as dJ322I12.1. In some embodiments, a BTBD9 gene is a human BTBD9 gene. An exemplary BTBD9 gene is represented by NCBI Gene ID No. 114781. Exemplary BTBD9 transcript sequences are represented by NCBI Ref. Seq. NM_052893 and provided herein as SEQ ID NO: 93. Exemplary amino acid sequences of a BTBD9 polypeptide are represented by NCBI Ref. Seq. NP_443125 and provided herein as SEQ ID NO: 188.

As used herein “C2orf73” refers to a gene encoding a C2orf73 mRNA or polypeptide. The C2orf73 gene encodes the uncharacterized protein C2orf73. In some embodiments, a C2orf73 gene is a human C2orf73 gene. An exemplary C2orf73 gene is represented by NCBI Gene ID No. 129852. Exemplary C2orf73 transcript sequences are represented by NCBI Ref. Seq. NM_001100396 and provided herein as SEQ ID NO: 95. Exemplary amino acid sequences of a C2orf73 polypeptide are represented by NCBI Ref. Seq. NP_001093866 and provided herein as SEQ ID NO: 190.

As used herein “CAPN14” refers to a gene encoding a CAPN14 mRNA or polypeptide. The CAPN14 gene encodes the calpain-14 protein. In some embodiments, a CAPN14 gene is a human CAPN14 gene. An exemplary CAPN14 gene is represented by NCBI Gene ID No. 440854. Exemplary CAPN14 transcript sequences are represented by NCBI Ref. Seq. NM_001145122 and provided herein as SEQ ID NO: 96. Exemplary amino acid sequences of a CAPN14 polypeptide are represented by NCBI Ref. Seq. NP_001138594 and provided herein as SEQ ID NO: 191.

As used herein “CARMIL1” refers to a gene encoding a CARMIL1 mRNA or polypeptide. The CARMIL1 gene encodes an F-actin-uncapping protein. CARMIL1 is also known as CARMIL, CARMIL1a, dJ501N12.1, dJ501N12.5, LRRC16, and LRRC16A. In some embodiments, a CARMIL1 gene is a human CARMIL1 gene. An exemplary CARMIL1 gene is represented by NCBI Gene ID No. 55604. Exemplary CARMIL1 transcript sequences are represented by NCBI Ref. Seq. NM_017640 and provided herein as SEQ ID NO: 97. Exemplary amino acid sequences of an CARMIL1 polypeptide are represented by NCBI Ref. Seq. NP_060110 and provided herein as SEQ ID NO: 192.

As used herein “CASP8” refers to a gene encoding a CASP8 mRNA or polypeptide. The CASP8 gene encodes the caspase-8 protein. CASP8 is also known as ALPS2B, CAP4, Casp-8, FLICE, MACH, and MCH5. In some embodiments, a CASP8 gene is a human CASP8 gene. An exemplary CASP8 gene is represented by NCBI Gene ID No. 841. Exemplary CASP8 transcript sequences are represented by NCBI Ref. Seq. NM_001228 and provided herein as SEQ ID NO: 98. Exemplary amino acid sequences of a CASP8 polypeptide are represented by NCBI Ref. Seq. NP_001219 and provided herein as SEQ ID NO: 193.

As used herein “CDC42BPA” refers to a gene encoding a CDC42BPA mRNA or polypeptide. The CDC42BPA gene encodes the serine/threonine-protein kinase MRCK alpha protein. CDC42BPA is also known as MRCK, MRCKA, and PK428. In some embodiments, a CDC42BPA gene is a human CDC42BPA gene. An exemplary CDC42BPA gene is represented by NCBI Gene ID No. 8476. Exemplary CDC42BPA transcript sequences are represented by NCBI Ref. Seq. NM_003607 and provided herein as SEQ ID NO: 99. Exemplary amino acid sequences of a CDC42BPA polypeptide are represented by NCBI Ref. Seq. NP_003598 and provided herein as SEQ ID NO: 194.

As used herein “CIB4” refers to a gene encoding a CIB4 mRNA or polypeptide. The CIB4 gene encodes the calcium and integrin-binding family member 4 protein. CIB4 is also known as KIP4. In some embodiments, a CIB4 gene is a human CIB4 gene. An exemplary CIB4 gene is represented by NCBI Gene ID No. 130106. Exemplary CIB4 transcript sequences are represented by NCBI Ref. Seq. NM_001029881 and provided herein as SEQ ID NO: 100. Exemplary amino acid sequences of a CIB4 polypeptide are represented by NCBI Ref. Seq. NP_001025052 and provided herein as SEQ ID NO: 195.

As used herein “CNTNAP5” refers to a gene encoding a CNTNAP5 mRNA or polypeptide. The CNTNAP5 gene encodes the contactin-associated protein-like 5 protein. CNTNAP5 is also known as caspr5. In some embodiments, a CNTNAP5 gene is a human CNTNAP5 gene. An exemplary CNTNAP5 gene is represented by NCBI Gene ID No. 129684. Exemplary CNTNAP5 transcript sequences are represented by NCBI Ref. Seq. NM_130773 and provided herein as SEQ ID NO: 101. Exemplary amino acid sequences of a CNTNAP5 polypeptide are represented by NCBI Ref. Seq. NP_570129 and provided herein as SEQ ID NO: 196.

As used herein “COL3A1” refers to a gene encoding a COL3A1 mRNA or polypeptide. The COL3A1 gene encodes the collagen alpha-1(III) chain protein. COL3A1 is also known as EDS4A, EDSVASC, and PMGEDSV. In some embodiments, a COL3A1 gene is a human COL3A1 gene. An exemplary COL3A1 gene is represented by NCBI Gene ID No. 1281. Exemplary COL3A1 transcript sequences are represented by NCBI Ref. Seq. NM_000090 and provided herein as SEQ ID NO: 103. Exemplary amino acid sequences of a COL3A1 polypeptide are represented by NCBI Ref. Seq. NP_000081 and provided herein as SEQ ID NO: 198.

As used herein “CPQ” refers to a gene encoding a CPQ mRNA or polypeptide. The CPQ gene encodes the carboxypeptidase Q protein. CPQ is also known as LDP and PGCP. In some embodiments, a CPQ gene is a human CPQ gene. An exemplary CPQ gene is represented by NCBI Gene ID No. 10404. Exemplary CPQ transcript sequences are represented by NCBI Ref. Seq. NM_016134 and provided herein as SEQ ID NO: 104. Exemplary amino acid sequences of a CPQ polypeptide are represented by NCBI Ref. Seq. NP_057218 and provided herein as SEQ ID NO: 199.

As used herein “CPSF7” refers to a gene encoding a CPSF7 mRNA or polypeptide. The CPSF7 gene encodes the cleavage and polyadenylation specificity factor subunit 7 protein. CPSF7 is also known as CFIm59. In some embodiments, a CPSF7 gene is a human CPSF7 gene. An exemplary CPSF7 gene is represented by NCBI Gene ID No. 79869. Exemplary CPSF7 transcript sequences are represented by NCBI Ref. Seq. NM_024811 and provided herein as SEQ ID NO: 105. Exemplary amino acid sequences of a CPSF7 polypeptide are represented by NCBI Ref. Seq. NP_079087 and provided herein as SEQ ID NO: 200.

As used herein “CREBBP” refers to a gene encoding a CREBBP mRNA or polypeptide. The CREBBP gene encodes the CREB-binding protein. CREBBP is also known as CBP, KAT3A, MKHK1, RSTS and RSTS1. In some embodiments, a CREBBP gene is a human CREBBP gene. An exemplary CREBBP gene is represented by NCBI Gene ID No. 1387. Exemplary CREBBP transcript sequences are represented by NCBI Ref. Seq. NM_004380 and provided herein as SEQ ID NO: 106. Exemplary amino acid sequences of a CREBBP polypeptide are represented by NCBI Ref. Seq. NP_004371 and provided herein as SEQ ID NO: 201.

As used herein “CTBP1” refers to a gene encoding a CTBP1 mRNA or polypeptide. The CTBP1 gene encodes the C-terminal-binding protein 1 protein. CTBP1 is also known as BARS and HADDTS. In some embodiments, a CTBP1 gene is a human CTBP1 gene. An exemplary CTBP1 gene is represented by NCBI Gene ID No. 1487. Exemplary CTBP1 transcript sequences are represented by NCBI Ref. Seq. NM_001328 and provided herein as SEQ ID NO: 107. Exemplary amino acid sequences of a CTBP1 polypeptide are represented by NCBI Ref. Seq. NP_001319 and provided herein as SEQ ID NO: 202.

As used herein “CTNND1” refers to a gene encoding a CTNND1 mRNA or polypeptide. The CTNND1 gene encodes the catenin delta-1 protein. CTNND1 is also known as BCDS2, CAS, CTNND, p120, p120(CAS),p120(CTN), P120CAS, and P120CTN. In some embodiments, a CTNND1 gene is a human CTNND1 gene. An exemplary CTNND1 gene is represented by NCBI Gene ID No. 1500. Exemplary CTNND1 transcript sequences are represented by NCBI Ref. Seq. NM_001331 and provided herein as SEQ ID NO: 108. Exemplary amino acid sequences of a CTNND1 polypeptide are represented by NCBI Ref. Seq. NP_001322 and provided herein as SEQ ID NO: 203.

As used herein “CYP51A1” refers to a gene encoding a CYP51A1 mRNA or polypeptide. The CYP51A1 gene encodes the lanosterol 14-alpha demethylase protein. CYP51A1 is also known as CP51; CYP51, CYPL1, LDM, P450-14DM, and P450L1. In some embodiments, a CYP51A1 gene is a human CYP51A1 gene. An exemplary CYP51A1 gene is represented by NCBI Gene ID No. 1595. Exemplary CYP51A1 transcript sequences are represented by NCBI Ref. Seq. NM_000786 and provided herein as SEQ ID NO: 109. Exemplary amino acid sequences of a CYP51A1 polypeptide are represented by NCBI Ref. Seq. NP_000777 and provided herein as SEQ ID NO: 204.

As used herein “CYS1” refers to a gene encoding a CYS1 mRNA or polypeptide. The CYS1 gene encodes the cystin-1 protein. In some embodiments, a CYS1 gene is a human CYS1 gene. An exemplary CYS1 gene is represented by NCBI Gene ID No. 192668. Exemplary CYS1 transcript sequences are represented by NCBI Ref. Seq. NM_001037160 and provided herein as SEQ ID NO: 110. Exemplary amino acid sequences of a CYS1 polypeptide are represented by NCBI Ref. Seq. NP_001032237 and provided herein as SEQ ID NO: 205.

As used herein “EPHA2” refers to a gene encoding an EPHA2 mRNA or polypeptide. The EPHA2 gene encodes the ephrin type-A receptor 2 protein. EPHA2 is also known as ARCC2, CTPA, CTPP1, CTRCT6, and ECK. In some embodiments, an EPHA2 gene is a human EPHA2 gene. An exemplary EPHA2 gene is represented by NCBI Gene ID No. 1969. Exemplary EPHA2 transcript sequences are represented by NCBI Ref. Seq. NM_004431 and provided herein as SEQ ID NO: 112. Exemplary amino acid sequences of an EPHA2 polypeptide are represented by NCBI Ref. Seq. NP_004422 and provided herein as SEQ ID NO: 207.

As used herein “FHOD3” refers to a gene encoding an FHOD3 mRNA or polypeptide. The FHOD3 gene encodes the FH1/FH2 domain-containing protein 3 protein. FHOD3 is also known as CMH28, FHOS2, and Formactin2. In some embodiments, an FHOD3 gene is a human FHOD3 gene. An exemplary FHOD3 gene is represented by NCBI Gene ID No. 80206. Exemplary FHOD3 transcript sequences are represented by NCBI Ref. Seq. NM_025135 and provided herein as SEQ ID NO: 113. Exemplary amino acid sequences of an FHOD3 polypeptide are represented by NCBI Ref. Seq. NP_079411 and provided herein as SEQ ID NO: 208.

As used herein “FILP1L” refers to a gene encoding an FILP1L mRNA or polypeptide. The FILP1L gene encodes the filamin A-interacting protein 1-like protein. FILP1L is also known as DOC-1; DOC1, GIP130 and GIP90. In some embodiments, a FILP1L gene is a human FILP1L gene. An exemplary FILP1L gene is represented by NCBI Gene ID No. 11259. Exemplary FILP1L transcript sequences are represented by NCBI Ref. Seq. NM_182909 and provided herein as SEQ ID NO: 114. Exemplary amino acid sequences of an FILP1L polypeptide are represented by NCBI Ref. Seq. NP_878913 and provided herein as SEQ ID NO: 209.

As used herein “GMCL1” refers to a gene encoding a GMCL1 mRNA or polypeptide. The GMCL1 gene encodes the germ cell-less protein-like 1 protein. GMCL1 is also known as BTBD13, GCL, GCL1, and SPATA29. In some embodiments, a GMCL1 gene is a human GMCL1 gene. An exemplary GMCL1 gene is represented by NCBI Gene ID No. 64395. Exemplary GMCL1 transcript sequences are represented by NCBI Ref. Seq. NM_178439 and provided herein as SEQ ID NO: 116. Exemplary amino acid sequences of a GMCL1 polypeptide are represented by NCBI Ref. Seq. NP_848526 and provided herein as SEQ ID NO: 210.

As used herein “GPN1” refers to a gene encoding a GPN1 mRNA or polypeptide. The GPN1 gene encodes the GPN-loop GTPase 1 protein. GPN1 is also known as ATPBD1A, MBDIN, NTPBP, RPAP4, and XAB1. In some embodiments, a GPN1 gene is a human GPN1 gene. An exemplary GPN1 gene is represented by NCBI Gene ID No. 11321. Exemplary GPN1 transcript sequences are represented by NCBI Ref. Seq. NM_007266 and provided herein as SEQ ID NO: 117. Exemplary amino acid sequences of a GPN1 polypeptide are represented by NCBI Ref. Seq. NP_009197 and provided herein as SEQ ID NO: 211.

As used herein “GPR113” refers to a gene encoding a GPR113 mRNA or polypeptide. The GPR113 gene encodes the adhesion G-protein coupled receptor F3 protein. GPR113 is also known as ADGRF3 and PGR23. In some embodiments, a GPR113 gene is a human GPR113 gene. An exemplary GPR113 gene is represented by NCBI Gene ID No. 165082. Exemplary GPR113 transcript sequences are represented by NCBI Ref. Seq. NM_153835 and provided herein as SEQ ID NO: 118. Exemplary amino acid sequences of a GPR113 polypeptide are represented by NCBI Ref. Seq. NP_722577 and provided herein as SEQ ID NO: 212.

As used herein “HADHA” refers to a gene encoding a HADHA mRNA or polypeptide. The HADHA gene encodes the trifunctional enzyme subunit alpha protein. HADHA is also known as ECHA, GBP, HADH, LCEH, LCHAD, MTPA, and TP-ALPHA. In some embodiments, a HADHA gene is a human HADHA gene. An exemplary HADHA gene is represented by NCBI Gene ID No. 3030. Exemplary HADHA transcript sequences are represented by NCBI Ref. Seq. NM_000182 and provided herein as SEQ ID NO: 120. Exemplary amino acid sequences of a HADHA polypeptide are represented by NCBI Ref. Seq. NP_000173 and provided herein as SEQ ID NO: 214.

As used herein “HS1BP3” refers to a gene encoding a HS1BP3 mRNA or polypeptide. The HS1BP3 gene encodes the HCLS1-binding protein 3 protein. HS1BP3 is also known as ETM2 and HS1-BP3. In some embodiments, a HS1BP3 gene is a human HS1BP3 gene. An exemplary HS1BP3 gene is represented by NCBI Gene ID No. 64342. Exemplary HS1BP3 transcript sequences are represented by NCBI Ref. Seq. NM_022460 and provided herein as SEQ ID NO: 121. Exemplary amino acid sequences of a HS1BP3 polypeptide are represented by NCBI Ref. Seq. NP_071905 and provided herein as SEQ ID NO: 215.

As used herein “INTS9” refers to a gene encoding a INTS9 mRNA or polypeptide. The INTS9 gene encodes the integrator complex subunit 9 protein. INTS9 is also known as CPSF2L, INT9, and RC74. In some embodiments, a INTS9 gene is a human INTS9 gene. An exemplary INTS9 gene is represented by NCBI Gene ID No. 55756. Exemplary INTS9 transcript sequences are represented by NCBI Ref. Seq. NM_018250 and provided herein as SEQ ID NO: 122. Exemplary amino acid sequences of a INTS9 polypeptide are represented by NCBI Ref. Seq. NP_060720 and provided herein as SEQ ID NO: 216.

As used herein “ITGA6” refers to a gene encoding a ITGA6 mRNA or polypeptide. The ITGA6 gene encodes the integrin alpha-6 protein. ITGA6 is also known as CD49f, ITGA6B, and VLA-6. In some embodiments, a ITGA6 gene is a human ITGA6 gene. An exemplary ITGA6 gene is represented by NCBI Gene ID No. 3655. Exemplary ITGA6 transcript sequences are represented by NCBI Ref. Seq. NM_000210 and provided herein as SEQ ID NO: 123. Exemplary amino acid sequences of a ITGA6 polypeptide are represented by NCBI Ref. Seq. NP_000201 and provided herein as SEQ ID NO: 217.

As used herein “KCTD18” refers to a gene encoding a KCTD18 mRNA or polypeptide. The KCTD18 gene encodes the BTB/POZ domain-containing protein KCTD18. KCTD18 is also known as 6530404F10Rik. In some embodiments, a KCTD18 gene is a human KCTD18 gene. An exemplary KCTD18 gene is represented by NCBI Gene ID No. 130535. Exemplary KCTD18 transcript sequences are represented by NCBI Ref. Seq. NM_152387 and provided herein as SEQ ID NO: 124. Exemplary amino acid sequences of a KCTD18 polypeptide are represented by NCBI Ref. Seq. NP_689600 and provided herein as SEQ ID NO: 218.

As used herein “KIF5C” refers to a gene encoding a KIF5C mRNA or polypeptide. The KIF5C gene encodes the kinesin heavy chain 5C protein. KIF5C is also known as CDCBM2, KINN, NKHC, NKHC-2, and NKHC2. In some embodiments, a KIF5C gene is a human KIF5C gene. An exemplary KIF5C gene is represented by NCBI Gene ID No. 3800. Exemplary KIF5C transcript sequences are represented by NCBI Ref. Seq. NM_004522 and provided herein as SEQ ID NO: 125. Exemplary amino acid sequences of a KIF5C polypeptide are represented by NCBI Ref. Seq. NP_004513 and provided herein as SEQ ID NO: 219.

As used herein “KLC4” refers to a gene encoding a KLC4 mRNA or polypeptide. The KLC4 gene encodes the kinesin light chain 4 protein. KLC4 is also known as bA387M24.3 and KNSL8. In some embodiments, a KLC4 gene is a human KLC4 gene. An exemplary KLC4 gene is represented by NCBI Gene ID No. 89953. Exemplary KLC4 transcript sequences are represented by NCBI Ref. Seq. NM_201521 and provided herein as SEQ ID NO: 126. Exemplary amino acid sequences of a KLC4 polypeptide are represented by NCBI Ref. Seq. NP_958929 and provided herein as SEQ ID NO: 220.

As used herein “LINC00535” refers to a gene encoding a LINC00535 mRNA. The LINC00535 gene encodes the CIBAR1 divergent transcript. LINC00535 is also known as CIBAR1-DT. In some embodiments, a LINC00535 gene is a human LINC00535 gene. An exemplary LINC00535 gene is represented by NCBI Gene ID No. 642924. Exemplary LINC00535 transcript sequences are represented by NCBI Ref. Seq. NR_033858 and provided herein as SEQ ID NO: 127.

As used herein “LRRFIP2” refers to a gene encoding a LRRFIP2 mRNA or polypeptide. The LRRFIP2 gene encodes the leucine-rich repeat flightless-interacting protein 2. LRRFIP2 is also known as HUFI-2. In some embodiments, a LRRFIP2 gene is a human LRRFIP2 gene. An exemplary LRRFIP2 gene is represented by NCBI Gene ID No. 9209. Exemplary LRRFIP2 transcript sequences are represented by NCBI Ref. Seq. NM_006309 and provided herein as SEQ ID NO: 128. Exemplary amino acid sequences of a LRRFIP2 polypeptide are represented by NCBI Ref. Seq. NP_006300 and provided herein as SEQ ID NO: 221.

As used herein “MAGOHB” refers to a gene encoding a MAGOHB mRNA or polypeptide. The MAGOHB gene encodes the protein mago nashi homolog 2 protein. MAGOHB is also known as mago, magoh, AND MGN2. In some embodiments, a MAGOHB gene is a human MAGOHB gene. An exemplary MAGOHB gene is represented by NCBI Gene ID No. 55110. Exemplary MAGOHB transcript sequences are represented by NCBI Ref. Seq. NM_018048 and provided herein as SEQ ID NO: 129. Exemplary amino acid sequences of a MAGOHB polypeptide are represented by NCBI Ref. Seq. NP_060518 and provided herein as SEQ ID NO: 222.

As used herein “MAMDC4” refers to a gene encoding a MAMDC4 mRNA or polypeptide. The MAMDC4 gene encodes the apical endosomal glycoprotein protein. MAMDC4 is also known as AEGP and EDTB. In some embodiments, a MAMDC4 gene is a human MAMDC4 gene. An exemplary MAMDC4 gene is represented by NCBI Gene ID No. 158056. Exemplary MAMDC4 transcript sequences are represented by NCBI Ref. Seq. NM_206920 and provided herein as SEQ ID NO: 130. Exemplary amino acid sequences of a MAMDC4 polypeptide are represented by NCBI Ref. Seq. NP_996803 and provided herein as SEQ ID NO: 223.

As used herein “MANBA” refers to a gene encoding a MANBA mRNA or polypeptide. The MANBA gene encodes the beta-mannosidase protein. MANBA is also known as MANB1. In some embodiments, a MANBA gene is a human MANBA gene. An exemplary MANBA gene is represented by NCBI Gene ID No. 4126. Exemplary MANBA transcript sequences are represented by NCBI Ref. Seq. NM_005908 and provided herein as SEQ ID NO: 131. Exemplary amino acid sequences of a MANBA polypeptide are represented by NCBI Ref. Seq. NP_005899 and provided herein as SEQ ID NO: 224.

As used herein “MAP3K9” refers to a gene encoding a MAP3K9 mRNA or polypeptide. The MAP3K9 gene encodes the mitogen-activated protein kinase kinase kinase 9 protein. MAP3K9 is also known as MEKK9, MLK1, and PRKE1. In some embodiments, a MAP3K9 gene is a human MAP3K9 gene. An exemplary MAP3K9 gene is represented by NCBI Gene ID No. 4293. Exemplary MAP3K9 transcript sequences are represented by NCBI Ref. Seq. NM_033141 and provided herein as SEQ ID NO: 132. Exemplary amino acid sequences of a MAP3K9 polypeptide are represented by NCBI Ref. Seq. NP_149132 and provided herein as SEQ ID NO: 225.

As used herein “MED13L” refers to a gene encoding a MED13L mRNA or polypeptide. The MED13L gene encodes the mediator of RNA polymerase II transcription subunit 13-likeprotein. MED13L is also known as MRFACD, PROSIT240, THRAP2 and TRAP240L. In some embodiments, a MED13L gene is a human MED13L gene. An exemplary MED13L gene is represented by NCBI Gene ID No. 23389. Exemplary MED13L transcript sequences are represented by NCBI Ref. Seq. NM_015335 and provided herein as SEQ ID NO: 133. Exemplary amino acid sequences of a MED13L polypeptide are represented by NCBI Ref. Seq. NP_056150 and provided herein as SEQ ID NO: 226.

As used herein “METTL25” refers to a gene encoding a METTL25 mRNA or polypeptide. The METTL25 gene encodes the probable methyltransferase-like protein 25 protein. METTL25 is also known as C12orf26. In some embodiments, a METTL25 gene is a human METTL25 gene. An exemplary METTL25 gene is represented by NCBI Gene ID No. 84190. Exemplary METTL25 transcript sequences are represented by NCBI Ref. Seq. NM_032230 and provided herein as SEQ ID NO: 134. Exemplary amino acid sequences of a METTL25 polypeptide are represented by NCBI Ref. Seq. NP_115606 and provided herein as SEQ ID NO: 227.

As used herein “MTBP” refers to a gene encoding a MTBP mRNA or polypeptide. The MTBP gene encodes the mdm2-binding protein protein. MTBP is also known as MDM2BP. In some embodiments, a MTBP gene is a human MTBP gene. An exemplary MTBP gene is represented by NCBI Gene ID No. 27085. Exemplary MTBP transcript sequences are represented by NCBI Ref. Seq. NM_022045 and provided herein as SEQ ID NO: 135. Exemplary amino acid sequences of a MTBP polypeptide are represented by NCBI Ref. Seq. NP_071328 and provided herein as SEQ ID NO: 228.

As used herein “MYH10” refers to a gene encoding a MYH10 mRNA or polypeptide. The MYH10 gene encodes the myosin-10 protein. MYH10 is also known as NMMHC-IIB and NMMHCB. In some embodiments, a MYH10 gene is a human MYH10 gene. An exemplary MYH10 gene is represented by NCBI Gene ID No. 4628. Exemplary MYH10 transcript sequences are represented by NCBI Ref. Seq. NM_005964 and provided herein as SEQ ID NO: 136. Exemplary amino acid sequences of a MYH10 polypeptide are represented by NCBI Ref. Seq. NP_005955 and provided herein as SEQ ID NO: 229.

As used herein “MYOSC” refers to a gene encoding a MYO5C mRNA or polypeptide. The MYO5C gene encodes the unconventional myosin-Vc protein. In some embodiments, a MYO5C gene is a human MYO5C gene. An exemplary MYO5C gene is represented by NCBI Gene ID No. 55930-Exemplary MYO5C transcript sequences are represented by NCBI Ref. Seq. NM_018728 and provided herein as SEQ ID NO: 137. Exemplary amino acid sequences of a MYO5C polypeptide are represented by NCBI Ref. Seq. NP_061198 and provided herein as SEQ ID NO: 230.

As used herein “NFIA” refers to a gene encoding a NFIA mRNA or polypeptide. The NFIA gene encodes the nuclear factor 1 A-type protein. NFIA is also known as BRMUTD, CTF, NF-I/A, NF1-A, NFI-A, and NFI-L. In some embodiments, a NFIA gene is a human NFIA gene. An exemplary NFIA gene is represented by NCBI Gene ID No. 4774. Exemplary NFIA transcript sequences are represented by NCBI Ref. Seq. NM_005595 and provided herein as SEQ ID NO: 138. Exemplary amino acid sequences of a NFIA polypeptide are represented by NCBI Ref. Seq. NP_005586 and provided herein as SEQ ID NO: 231.

As used herein “NINJ2” refers to a gene encoding a NINJ2 mRNA or polypeptide. The NINJ2 gene encodes the ninjurin-2 protein. In some embodiments, a NINJ2 gene is a human NINJ2 gene. An exemplary NINJ2 gene is represented by NCBI Gene ID No. 4815. Exemplary NINJ2 transcript sequences are represented by NCBI Ref. Seq. NM_016533 and provided herein as SEQ ID NO: 139. Exemplary amino acid sequences of a NINJ2 polypeptide are represented by NCBI Ref. Seq. NP_057617 and provided herein as SEQ ID NO: 232.

As used herein “OPRM1” refers to a gene encoding a OPRM1 mRNA or polypeptide. The OPRM1 gene encodes a mu-type opioid receptor protein. OPRM1 is also known as LMOR, M-OR-1, MOP, MOR, MOR1, and OPRM. In some embodiments, a OPRM1 gene is a human OPRM1 gene. An exemplary OPRM1 gene is represented by NCBI Gene ID No. 4988. Exemplary OPRM1 transcript sequences are represented by NCBI Ref. Seq. NM_001008503 and provided herein as SEQ ID NO: 140. Exemplary amino acid sequences of a OPRM1 polypeptide are represented by NCBI Ref. Seq. NP_001008503 and provided herein as SEQ ID NO: 233.

As used herein “OTX1” refers to a gene encoding a OTX1 mRNA or polypeptide. The OTX1 gene encodes the homeobox protein OTX1 protein. In some embodiments, a OTX1 gene is a human OTX1 gene. An exemplary OTX1 gene is represented by NCBI Gene ID No. 5013. Exemplary OTX1 transcript sequences are represented by NCBI Ref. Seq. NM_014562 and provided herein as SEQ ID NO: 141. Exemplary amino acid sequences of a OTX1 polypeptide are represented by NCBI Ref. Seq. NP_055377 and provided herein as SEQ ID NO: 234.

As used herein “PAQR4” refers to a gene encoding a PAQR4 mRNA or polypeptide. The PAQR4 gene encodes the progestin and adipoQ receptor family member 4 protein. In some embodiments, a PAQR4 gene is a human PAQR4 gene. An exemplary PAQR4 gene is represented by NCBI Gene ID No. 124222. Exemplary PAQR4 transcript sequences are represented by NCBI Ref. Seq. NM_152341 and provided herein as SEQ ID NO: 142. Exemplary amino acid sequences of a PAQR4 polypeptide are represented by NCBI Ref. Seq. NP_689554 and provided herein as SEQ ID NO: 235.

As used herein “PDCD10” refers to a gene encoding a PDCD10 mRNA or polypeptide. The PDCD10 gene encodes the programmed cell death protein 10 protein. PDCD10 is also known as CCM3 and TFAR15. In some embodiments, a PDCD10 gene is a human PDCD10 gene. An exemplary PDCD10 gene is represented by NCBI Gene ID No. 11235. Exemplary PDCD10 transcript sequences are represented by NCBI Ref. Seq. NM_007217 and provided herein as SEQ ID NO: 143. Exemplary amino acid sequences of a PDCD10 polypeptide are represented by NCBI Ref. Seq. NP_009148 and provided herein as SEQ ID NO: 236.

As used herein “PDE3A” refers to a gene encoding a PDE3A mRNA or polypeptide. The PDE3A gene encodes the cGMP-inhibited 3′, 5′-cyclic phosphodiesterase A protein. PDE3A is also known as CGI-PDE, CGI-PDE A, CGI-PDE-A, and HTNB. In some embodiments, a PDE3A gene is a human PDE3A gene. An exemplary PDE3A gene is represented by NCBI Gene ID No. 5139. Exemplary PDE3A transcript sequences are represented by NCBI Ref. Seq. NM_000921 and provided herein as SEQ ID NO: 144. Exemplary amino acid sequences of a PDE3A polypeptide are represented by NCBI Ref. Seq. NP_000912 and provided herein as SEQ ID NO: 237.

As used herein “PELI1” refers to a gene encoding a PELI1 mRNA or polypeptide. The PELI1 gene encodes the E3 ubiquitin-protein ligase pellino homolog 1 protein. In some embodiments, a PELI1 gene is a human PELI1 gene. An exemplary PELI1 gene is represented by NCBI Gene ID No. 57162. Exemplary PELI1 transcript sequences are represented by NCBI Ref. Seq. NM_020651 and provided herein as SEQ ID NO: 145. Exemplary amino acid sequences of a PELI1 polypeptide are represented by NCBI Ref. Seq. NP_065702 and provided herein as SEQ ID NO: 238.

As used herein “PLEC” refers to a gene encoding a PLEC mRNA or polypeptide. The PLEC gene encodes a plectin protein. PLEC is also known as: EBS1; EBS5A; EBS5B; EBS5C; EBS5D; EBSMD; EBSND; EBSO; EBSOG; EBSPA; HD1; LGMD2Q; LGMDR17; PCN; PLEC1; PLEC1b; and PLTN. In some embodiments, a PLEC gene is a human PLEC gene. An exemplary PLEC gene is represented by NCBI Gene ID No. 5339. Exemplary PLEC transcript sequences are represented by NCBI Ref. Seq. NM_000445 and provided herein as SEQ ID NO: 146. Exemplary amino acid sequences of a PLEC polypeptide are represented by NCBI Ref. Seq. NP_000436 and provided herein as SEQ ID NO: 239.

As used herein “PTGER4” refers to a gene encoding a PTGER4 mRNA or polypeptide. The PTGER4 gene encodes the prostaglandin E2 receptor EP4 subtype protein. PTGER4 is also known as EP4 and EP4R. In some embodiments, a PTGER4 gene is a human PTGER4 gene. An exemplary PTGER4 gene is represented by NCBI Gene ID No. 5734. Exemplary PTGER4 transcript sequences are represented by NCBI Ref. Seq. NM_000958 and provided herein as SEQ ID NO: 147. Exemplary amino acid sequences of a PTGER4 polypeptide are represented by NCBI Ref. Seq. NP_000949 and provided herein as SEQ ID NO: 240.

As used herein “PTPRJ” refers to a gene encoding a PTPRJ mRNA or polypeptide. The PTPRJ gene encodes a receptor-type tyrosine-protein phosphatase eta protein. PTPRJ is also known as: CD148; DEP1; HPTPeta; R-PTP-ETA; and SCC1. In some embodiments, a PTPRJ gene is a human PTPRJ gene. An exemplary PTPRJ gene is represented by NCBI Gene ID No. 5795-Exemplary PTPRJ transcript sequences are represented by NCBI Ref. Seq. NM_002843 and provided herein as SEQ ID NO: 148. Exemplary amino acid sequences of a PTPRJ polypeptide are represented by NCBI Ref. Seq. NP_002834 and provided herein as SEQ ID NO: 241.

As used herein “QKI” refers to a gene encoding a QKI mRNA or polypeptide. The QKI gene encodes protein quaking. QKI is also known as: Hqk; hqkI; QK; QK1; and QK3. In some embodiments, a QKI gene is a human QKI gene. An exemplary QKI gene is represented by NCBI Gene ID No. 9444. Exemplary QKI transcript sequences are represented by NCBI Ref. Seq. NM_006775 and provided herein as SEQ ID NO: 149. Exemplary amino acid sequences of a QKI polypeptide are represented by NCBI Ref. Seq. NP_006766 and provided herein as SEQ ID NO: 242.

As used herein “RPS6KA5” refers to a gene encoding a RPS6KA5 mRNA or polypeptide. The RPS6KA5 gene encodes a ribosomal protein S6 kinase alpha-5. RPS6KA5 is also known as: MSK1; MSPK1; and RLPK. In some embodiments, a RPS6KA5 gene is a human RPS6KA5 gene. An exemplary RPS6KA5 gene is represented by NCBI Gene ID No. 9252. Exemplary RPS6KA5 transcript sequences are represented by NCBI Ref. Seq. NM_004755 and provided herein as SEQ ID NO: 152. Exemplary amino acid sequences of a RPS6KA5 polypeptide are represented by NCBI Ref. Seq. NP_004746 and provided herein as SEQ ID NO: 245.

As used herein “SASH1” refers to a gene encoding a SASH1 mRNA or polypeptide. The SASH1 gene encodes a SAM and SH3 domain-containing protein 1. SASH1 is also known as: CAPOK; dJ323M4.1; DUH1; and SH3D6A. In some embodiments, a SASH1 gene is a human SASH1 gene. An exemplary SASH1 gene is represented by NCBI Gene ID No. 23328. Exemplary SASH1 transcript sequences are represented by NCBI Ref. Seq. NM_015278 and provided herein as SEQ ID NO: 153. Exemplary amino acid sequences of a SASH1 polypeptide are represented by NCBI Ref. Seq. NP_056093 and provided herein as SEQ ID NO: 246.

As used herein “SEC16B” refers to a gene encoding a SEC16B mRNA or polypeptide. The SEC16B gene encodes a protein transport protein. SEC16B is also known as: LZTR2; PGPR-p117; RGPR; RGPR-p117; and SEC16S. In some embodiments, a SEC16B gene is a human SEC16B gene. An exemplary SEC16B gene is represented by NCBI Gene ID No. 89866. Exemplary SEC16B transcript sequences are represented by NCBI Ref. Seq. NM_033127 and provided herein as SEQ ID NO: 154. Exemplary amino acid sequences of a SEC16B polypeptide are represented by NCBI Ref. Seq. NP_149118 and provided herein as SEQ ID NO: 247.

As used herein “SKAP1” refers to a gene encoding a SKAP1 mRNA or polypeptide. The SKAP1 gene encodes a src kinase-associated phosphoprotein 1. SKAP1 is also known as: HEL-S-81p; SCAP1; and SKAP55. In some embodiments, a SKAP1 gene is a human SKAP1 gene. An exemplary SKAP1 gene is represented by NCBI Gene ID No. 8631. Exemplary SKAP1 transcript sequences are represented by NCBI Ref. Seq. NM_003726 and provided herein as SEQ ID NO: 156. Exemplary amino acid sequences of a SKAP1 polypeptide are represented by NCBI Ref. Seq. NP_003717 and provided herein as SEQ ID NO: 249.

As used herein “SLC25A13” refers to a gene encoding a SLC25A13 mRNA or polypeptide. The SLC25A13 gene encodes a calcium-binding mitochondrial carrier protein Aralar2. SLC25A13 is also known as: ARALAR2; CITRIN; CTLN2; and NICCD. In some embodiments, a SLC25A13 gene is a human SLC25A13 gene. An exemplary SLC25A13 gene is represented by NCBI Gene ID No. 10165. Exemplary SLC25A13 transcript sequences are represented by NCBI Ref. Seq. NM_014251 and provided herein as SEQ ID NO: 158. Exemplary amino acid sequences of a SLC25A13 polypeptide are represented by NCBI Ref. Seq. NP_055066 and provided herein as SEQ ID NO: 251.

As used herein “SLC30A6” refers to a gene encoding a SLC30A6 mRNA or polypeptide. The SLC30A6 gene encodes a zinc transporter 6 protein. SLC30A6 is also known as: MST103; MSTP103; and ZNT6. In some embodiments, a SLC30A6 gene is a human SLC30A6 gene. An exemplary SLC30A6 gene is represented by NCBI Gene ID No. 55676. Exemplary SLC30A6 transcript sequences are represented by NCBI Ref. Seq. NM_017964 and provided herein as SEQ ID NO: 159. Exemplary amino acid sequences of a SLC30A6 polypeptide are represented by NCBI Ref. Seq. NP_060434 and provided herein as SEQ ID NO: 252.

As used herein “SNX17” refers to a gene encoding a SNX17 mRNA or polypeptide. The SNX17 gene encodes a sorting nexin-17 protein. In some embodiments, a SNX17 gene is a human SNX17 gene. An exemplary SNX17 gene is represented by NCBI Gene ID No. 9784. Exemplary SNX17 transcript sequences are represented by NCBI Ref. Seq. NM_014748 and provided herein as SEQ ID NO: 161. Exemplary amino acid sequences of a SNX17 polypeptide are represented by NCBI Ref. Seq. NP_055563 and provided herein as SEQ ID NO: 254.

As used herein “SOX13” refers to a gene encoding a SOX13 mRNA or polypeptide. The SOX13 gene encodes a transcription factor. SOX13 is also known as: ICA12 and Sox-13. In some embodiments, a SOX13 gene is a human SOX13 gene. An exemplary SOX13 gene is represented by NCBI Gene ID No. 9580. Exemplary SOX13 transcript sequences are represented by NCBI Ref. Seq. NM_005686 and provided herein as SEQ ID NO: 162. Exemplary amino acid sequences of a SOX13 polypeptide are represented by NCBI Ref. Seq. NP_005677 and provided herein as SEQ ID NO: 255.

As used herein “SRSF7” refers to a gene encoding a SRSF7 mRNA or polypeptide. The SRSF7 gene encodes a serine/arginine-rich splicing factor 7. SRSF7 is also known as: 9G8; AAG3; and SFRS7. In some embodiments, a SRSF7 gene is a human SRSF7 gene. An exemplary SRSF7 gene is represented by NCBI Gene ID No. 6432. Exemplary SRSF7 transcript sequences are represented by NCBI Ref. Seq. NM_001031684 and provided herein as SEQ ID NO: 163. Exemplary amino acid sequences of a SRSF7 polypeptide are represented by NCBI Ref. Seq. NP_001026854 and provided herein as SEQ ID NO: 256.

As used herein “TANGO6” refers to a gene encoding a TANGO6 mRNA or polypeptide. The TANGO6 gene encodes a transport and Golgi organization protein 6. TANGO6 is also known as: TMCO7. In some embodiments, a TANGO6 gene is a human TANGO6 gene. An exemplary TANGO6 gene is represented by NCBI Gene ID No. 79613. Exemplary TANGO6 transcript sequences are represented by NCBI Ref. Seq. NM_024562 and provided herein as SEQ ID NO: 164. Exemplary amino acid sequences of a TANGO6 polypeptide are represented by NCBI Ref. Seq. NP_078838 and provided herein as SEQ ID NO: 257.

As used herein “TG” refers to a gene encoding a TG mRNA or polypeptide. The TG gene encodes a thyroglobulin precursor. TG is also known as: AITD3 and TGN. In some embodiments, a TG gene is a human TG gene. An exemplary TG gene is represented by NCBI Gene ID No. 7038. Exemplary TG transcript sequences are represented by NCBI Ref. Seq. NM_003235 and provided herein as SEQ ID NO: 165. Exemplary amino acid sequences of a TG polypeptide are represented by NCBI Ref. Seq. NP_003226 and provided herein as SEQ ID NO: 258.

As used herein “TMCO3” refers to a gene encoding a TMCO3 mRNA or polypeptide. The TMCO3 gene encodes a transmembrane and coiled-coil domain-containing protein 3. TMCO3 is also known as: C13orf11. In some embodiments, a TMCO3 gene is a human TMCO3 gene. An exemplary TMCO3 gene is represented by NCBI Gene ID No. 55002. Exemplary TMCO3 transcript sequences are represented by NCBI Ref. Seq. NM_017905 and provided herein as SEQ ID NO: 166. Exemplary amino acid sequences of a TMCO3 polypeptide are represented by NCBI Ref. Seq. NP_060375 and provided herein as SEQ ID NO: 259.

As used herein “TNS3” refers to a gene encoding a TNS3 mRNA or polypeptide. The TNS3 gene encodes a tensin-3 protein. TNS3 is also known as: TEM6 and TENS1. In some embodiments, a TNS3 gene is a human TNS3 gene. An exemplary TNS3 gene is represented by NCBI Gene ID No. 64759. Exemplary TNS3 transcript sequences are represented by NCBI Ref. Seq. NM_022748 and provided herein as SEQ ID NO: 167. Exemplary amino acid sequences of a TNS3 polypeptide are represented by NCBI Ref. Seq. NP_073585 and provided herein as SEQ ID NO: 260.

As used herein “TRIM24” refers to a gene encoding a TRIM24 mRNA or polypeptide. The TRIM24 gene encodes a transcription intermediary factor 1-alpha. TRIM24 is also known as: hTIF1; PTC6; RNF82; TF1A; TIF1; TIF1A; and TIF1 ALPHA. In some embodiments, a TRIM24 gene is a human TRIM24 gene. An exemplary TRIM24 gene is represented by NCBI Gene ID No. 8805. Exemplary TRIM24 transcript sequences are represented by NCBI Ref. Seq. NM_003852 and provided herein as SEQ ID NO: 169. Exemplary amino acid sequences of a TRIM24 polypeptide are represented by NCBI Ref. Seq. NP_003843 and provided herein as SEQ ID NO: 262.

As used herein “TTC28” refers to a gene encoding a TTC28 mRNA or polypeptide. The TTC28 gene encodes a tetratricopeptide repeat protein 28. TTC28 is also known as: TPRBK. In some embodiments, a TTC28 gene is a human TTC28 gene. An exemplary TTC28 gene is represented by NCBI Gene ID No. 23331. Exemplary TTC28 transcript sequences are represented by NCBI Ref. Seq. NM_001145418 and provided herein as SEQ ID NO: 170. Exemplary amino acid sequences of a TTC28 polypeptide are represented by NCBI Ref. Seq. NP_001138890 and provided herein as SEQ ID NO: 263.

As used herein “UBE2L3” refers to a gene encoding a UBE2L3 mRNA or polypeptide. The UBE2L3 gene encodes an ubiquitin-conjugating enzyme E2 L3. UBE2L3 is also known as: E2-F1; L-UBC; UBCH7; and UbcM4. In some embodiments, a UBE2L3 gene is a human UBE2L3 gene. An exemplary UBE2L3 gene is represented by NCBI Gene ID No. 7332. Exemplary UBE2L3 transcript sequences are represented by NCBI Ref. Seq. NM_003347 and provided herein as SEQ ID NO: 172. Exemplary amino acid sequences of a UBE2L3 polypeptide are represented by NCBI Ref. Seq. NP_003338 and provided herein as SEQ ID NO: 265.

As used herein “UBE3B” refers to a gene encoding a UBE3B mRNA or polypeptide. The UBE3B gene encodes an ubiquitin-protein ligase E3B. UBE3B is also known as: BPIDS and KOS. In some embodiments, a UBE3B gene is a human UBE3B gene. An exemplary UBE3B gene is represented by NCBI Gene ID No. 89910. Exemplary UBE3B transcript sequences are represented by NCBI Ref. Seq. NM_130466 and provided herein as SEQ ID NO: 173. Exemplary amino acid sequences of a UBE3B polypeptide are represented by NCBI Ref. Seq. NP_569733 and provided herein as SEQ ID NO: 266.

As used herein “UTRN” refers to a gene encoding a UTRN mRNA or polypeptide. The UTRN gene encodes an utrophin protein. UTRN is also known as: DMDL; DRP; and DRP1. In some embodiments, a UTRN gene is a human UTRN gene. An exemplary UTRN gene is represented by NCBI Gene ID No. 7402. Exemplary UTRN transcript sequences are represented by NCBI Ref. Seq. NM_007124 and provided herein as SEQ ID NO: 174. Exemplary amino acid sequences of a UTRN polypeptide are represented by NCBI Ref. Seq. NP_009055 and provided herein as SEQ ID NO: 267.

As used herein “VASP” refers to a gene encoding a VASP mRNA or polypeptide. The VASP gene encodes a vasodilator-stimulated phosphoprotein. In some embodiments, a VASP gene is a human VASP gene. An exemplary VASP gene is represented by NCBI Gene ID No. 7408. Exemplary VASP transcript sequences are represented by NCBI Ref. Seq. NM_003370 and provided herein as SEQ ID NO: 175. Exemplary amino acid sequences of a VASP polypeptide are represented by NCBI Ref. Seq. NP_003361 and provided herein as SEQ ID NO: 268.

As used herein “WDR92” refers to a gene encoding a WDR92 mRNA or polypeptide. The WDR92 gene encodes a dynein axonemal assembly factor 10. WDR92 is also known as: DNAAF10. In some embodiments, a WDR92 gene is a human WDR92 gene. An exemplary WDR92 gene is represented by NCBI Gene ID No. 116143. Exemplary WDR92 transcript sequences are represented by NCBI Ref. Seq. NM_138458 and provided herein as SEQ ID NO: 176. Exemplary amino acid sequences of a WDR92 polypeptide are represented by NCBI Ref. Seq. NP_612467 and provided herein as SEQ ID NO: 269.

As used herein “YPEL5” refers to a gene encoding an YPEL5 mRNA or polypeptide. The YPEL5 gene encodes a protein yippee-like 5. YPEL5 is also known as: CGI-127. In some embodiments, an YPEL5 gene is a human YPEL5 gene. An exemplary YPEL5 gene is represented by NCBI Gene ID No. 51646. Exemplary YPEL5 transcript sequences are represented by NCBI Ref. Seq. NM_016061 and provided herein as SEQ ID NO: 177. Exemplary amino acid sequences of an YPEL5 polypeptide are represented by NCBI Ref. Seq. NP_057145 and provided herein as SEQ ID NO: 270.

As used herein “ZNF446” refers to a gene encoding a ZNF446 mRNA or polypeptide. The ZNF446 gene encodes a zinc finger protein 446. ZNF446 is also known as: ZKSCAN20; ZSCAN30; and ZSCAN52. In some embodiments, a ZNF446 gene is a human ZNF446 gene. An exemplary ZNF446 gene is represented by NCBI Gene ID No. 55663. Exemplary ZNF446 transcript sequences are represented by NCBI Ref. Seq. NM_017908 and provided herein as SEQ ID NO: 178. Exemplary amino acid sequences of a ZNF446 polypeptide are represented by NCBI Ref. Seq. NP_060378 and provided herein as SEQ ID NO: 271.

As used herein “ZNF454” refers to a gene encoding a ZNF454 mRNA or polypeptide. The ZNF454 gene encodes a zinc finger protein 454. In some embodiments, a ZNF454 gene is a human ZNF454 gene. An exemplary ZNF454 gene is represented by NCBI Gene ID No. 285676. Exemplary ZNF454 transcript sequences are represented by NCBI Ref. Seq. NM_182594 and provided herein as SEQ ID NO: 179. Exemplary amino acid sequences of a ZNF454 polypeptide are represented by NCBI Ref. Seq. NP_872400 and provided herein as SEQ ID NO: 272.

As used herein “ZNF513” refers to a gene encoding a ZNF513 mRNA or polypeptide. The ZNF513 gene encodes a zinc finger protein 513. ZNF513 is also known as: HMFT0656; RP58; and Zfp513. In some embodiments, a ZNF513 gene is a human ZNF513 gene. An exemplary ZNF513 gene is represented by NCBI Gene ID No. 130557. Exemplary ZNF513 transcript sequences are represented by NCBI Ref. Seq. NM_144631 and provided herein as SEQ ID NO: 180. Exemplary amino acid sequences of a ZNF513 polypeptide are represented by NCBI Ref. Seq. NP_653232 and provided herein as SEQ ID NO: 273.

As used herein “ZSWIM2” refers to a gene encoding a ZSWIM2 mRNA or polypeptide. The ZSWIM2 gene encodes an E3 ubiquitin-protein ligase. ZSWIM2 is also known as: MEX and ZZZ2. In some embodiments, a ZSWIM2 gene is a human ZSWIM2 gene. An exemplary ZSWIM2 gene is represented by NCBI Gene ID No. 151112. Exemplary ZSWIM2 transcript sequences are represented by NCBI Ref. Seq. NM_182521 and provided herein as SEQ ID NO: 181. Exemplary amino acid sequences of a ZSWIM2 polypeptide are represented by NCBI Ref. Seq. NP_872327 and provided herein as SEQ ID NO: 274.

### (i) Exemplary ALK Fusion Nucleic Acid Molecules

In some aspects, provided herein are ALK fusion nucleic acid molecules comprising at least a portion of an ALK gene fused to at least a portion of another gene.

In some embodiments, an ALK fusion nucleic acid molecule of the disclosure comprises at least a portion of ALK and at least a portion of an ABCB11, ACTN4, AGAP1, APH1A, AZI2, BTBD9, C2orf73, CAPN14, CARMIL1, CASP8, CDC42BPA, CIB4, CNTNAP5, COL3A1, CPQ, CPSF7, CREBBP, CTBP1, CTNND1, CYP51A1, CYS1, EPHA2, FHOD3, FILIP1L, GMCL1, GPN1, GPR113, HADHA, HS1BP3, INTS9, ITGA6, KCTD18, KIF5C, KLC4, LINC00535, LRRFIP2, MAGOHB, MAMDC4, MANBA, MAP3K9, MED13L, METTL25, MTBP, MYH10, MYO5C, NFIA, NINJ2, OPRM1, OTX1, PAQR4, PDCD10, PDE3A, PELI1, PLEC, PTGER4, PTPRJ, QKI, RPS6KA5, SASH1, SEC16B, SKAP1, SLC25A13, SLC30A6, SNX17, SOX13, SRSF7, TANGO6, TG, TMCO3, TNS3, TRIM24, TTC28, UBE2L3, UBE3B, UTRN, VASP, WDR92, YPEL5, ZNF446, ZNF454, ZNF513, or ZSWIM2 gene, or a gene listed in Table 1.

TABLE 1
Gene fusion partners.

| | | | | | | |
|---|---|---|---|---|---|---|
| ABCB11 | COL3A1 | GPR113 | MAP3K9 | PELI1 | SRSF7 | ZNF446 |
| ACTN4 | CPQ | HADHA | MED13L | PLEC | TANGO6 | ZNF454 |
| AGAP1 | CPSF7 | HS1BP3 | METTL25 | PTGER4 | TG | ZNF513 |
| APH1A | CREBBP | INTS9 | MTBP | PTPRJ | TMCO3 | ZSWIM2 |
| AZI2 | CTBP1 | ITGA6 | MYH10 | QKI | TNS3 | |
| BTBD9 | CTNND1 | KCTD18 | MYO5C | RPS6KA5 | TRIM24 | |
| C2orf73 | CYP51A1 | KIF5C | NFIA | SASH1 | TTC28 | |
| CAPN14 | CYS1 | KLC4 | NINJ2 | SEC16B | UBE2L3 | |
| CARMIL1 | EPHA2 | LINC00535 | OPRM1 | SKAP1 | UBE3B | |
| CASP8 | FHOD3 | LRRFIP2 | OTX1 | SLC25A13 | UTRN | |
| CDC42BPA | FILIP1L | MAGOHB | PAQR4 | SLC30A6 | VASP | |
| CIB4 | GMCL1 | MAMDC4 | PDCD10 | SNX17 | WDR92 | |
| CNTNAP5 | GPN1 | MANBA | PDE3A | SOX13 | YPEL5 | |

For example, in some embodiments, the ALK fusion nucleic acid molecule is selected from an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule.

In some embodiments, the ALK fusion nucleic acid molecule of the disclosure comprises at least a portion of an ALK gene and at least a portion of an ABCB11, ACTN4, AGAP1, APH1A, AZI2, BTBD9, C2orf73, CAPN14, CARMIL1, CASP8, CDC42BPA, CIB4, CNTNAP5, COL3A1, CPQ, CPSF7, CREBBP, CTBP1, CTNND1, CYP51A1, CYS1, EPHA2, FHOD3, FILIP1L, GMCL1, GPN1, GPR113, HADHA, HS1BP3, INTS9, ITGA6, KCTD18, KIF5C, KLC4, LINC00535, LRRFIP2, MAGOHB, MAMDC4, MANBA, MAP3K9, MED13L, METTL25, MTBP, MYH10, MYO5C, NFIA, NINJ2, OPRM1, OTX1, PAQR4, PDCD10, PDE3A, PELI1, PLEC, PTGER4, PTPRJ, QKI, RPS6KA5, SASH1, SEC16B, SKAP1, SLC25A13, SLC30A6, SNX17, SOX13, SRSF7, TANGO6, TG, TMCO3, TNS3, TRIM24, TTC28, UBE2L3, UBE3B, UTRN, VASP, WDR92, YPEL5, ZNF446, ZNF454, ZNF513, or ZSWIM2 gene, wherein the order of the genes in the fusion in the 5′ to 3′ direction is as indicated in Table 2.

TABLE 2
Order of fused genes in exemplary
ALK fusion nucleic acid molecules.

| ALK Fusion Nucleic Acid | | |
|---|---|---|
| Molecule | 5′ Gene | 3′ Gene |
| ALK-COL3A1 | COL3A1 | ALK |
| ALK-CDC42BPA | CDC42BPA | ALK |
| ALK-EPHA2 | EPHA2 | ALK |
| ALK-MYO5C | MYO5C | ALK |
| ALK-TRIM24 | TRIM24 | ALK |
| ALK-SKAP1 | SKAP1 | ALK |
| ALK-UBE3B | UBE3B | ALK |
| ALK-TNS3 | TNS3 | ALK |
| ALK-C2orf73 | C2orf73 | ALK |
| ALK-AZI2 | AZI2 | ALK |
| ALK-MANBA | MANBA | ALK |
| ALK-CNTNAP5 | CNTNAP5 | ALK |
| ALK-TANGO6 | TANGO6 | ALK |
| ALK-NFIA | NFIA | ALK |
| ALK-RPS6KA5 | RPS6KA5 | ALK |
| ALK-TG | TG | ALK |
| ALK-LRRFIP2 | LRRFIP2 | ALK |
| ALK-MYO5C | MYO5C | ALK |
| ALK-AGAP1 | AGAP1 | ALK |
| ALK-MED13L | ALK | MED13L |
| ALK-MTBP | ALK | MTBP |
| ALK-SLC30A6 | ALK | SLC30A6 |
| ALK-GMCL1 | ALK | GMCL1 |
| ALK-AGAP1 | ALK | AGAP1 |
| ALK-ZNF454 | ALK | ZNF454 |
| ALK-TTC28 | TTC28 | ALK |
| ALK-NINJ2 | NINJ2 | ALK |
| ALK-UTRN | UTRN | ALK |
| ALK-ACTN4 | ACTN4 | ALK |
| ALK-CPSF7 | CPSF7 | ALK |
| ALK-SLC25A13 | SLC25A13 | ALK |
| ALK-CTNND1 | CTNND1 | ALK |
| ALK-KLC4 | KLC4 | ALK |
| ALK-BTBD9 | BTBD9 | ALK |
| ALK-CPQ | CPQ | ALK |
| ALK-KIF5C | KIF5C | ALK |
| ALK-MAGOHB | MAGOHB | ALK |
| ALK-COL3A1 | COL3A1 | ALK |
| ALK-OPRM1 | OPRM1 | ALK |
| ALK-GPN1 | GPN1 | ALK |
| ALK-SEC16B | SEC16B | ALK |
| ALK-UBE2L3 | UBE2L3 | ALK |
| ALK-METTL25 | METTL25 | ALK |
| ALK-CYS1 | CYS1 | ALK |
| ALK-ABCB11 | ABCB11 | ALK |
| ALK-INTS9 | INTS9 | ALK |
| ALK-CIB4 | CIB4 | ALK |
| ALK-WDR92 | WDR92 | ALK |
| ALK-OTX1 | ALK | OTX1 |
| ALK-PDCD10 | ALK | PDCD10 |
| ALK-PTGER4 | ALK | PTGER4 |
| ALK-PTPRJ | ALK | PTPRJ |
| ALK-ZSWIM2 | ALK | ZSWIM2 |
| ALK-FHOD3 | ALK | FHOD3 |
| ALK-FILIP1L | ALK | FILP1L |
| ALK-ITGA6 | ALK | ITGA6 |
| ALK-KCTD18 | ALK | KCTD18 |
| ALK-MAMDC4 | ALK | MAMDC4 |
| ALK-PELI1 | PELI1 | ALK |
| ALK-LINC00535 | LINC00535 | ALK |
| ALK-CTBP1 | CTBP1 | ALK |
| ALK-CARMIL1 | CARMIL1 | ALK |
| ALK-ZNF513 | ZNF513 | ALK |
| ALK-TMCO3 | TMCO3 | ALK |
| ALK-SRSF7 | SRSF7 | ALK |
| ALK-CASP8 | CASP8 | ALK |
| ALK-CYP51A1 | CYP51A1 | ALK |
| ALK-GPR113 | GPR113 | ALK |
| ALK-HADHA | HADHA | ALK |
| ALK-KIF5C | KIF5C | ALK |
| ALK-LRRFIP2 | LRRFIP2 | ALK |
| ALK-MYH10 | MYH10 | ALK |
| ALK-PDE3A | PDE3A | ALK |
| ALK-PLEC | PLEC | ALK |
| ALK-QKI | QKI | ALK |
| ALK-SASH1 | SASH1 | ALK |
| ALK-SRSF7 | SRSF7 | ALK |
| ALK-VASP | VASP | ALK |
| ALK-ZNF446 | ZNF446 | ALK |
| ALK-SOX13 | SOX13 | ALK |
| ALK-YPEL5 | YPEL5 | ALK |
| ALK-CAPN14 | ALK | CAPN14 |
| ALK-MAP3K9 | ALK | MAP3K9 |
| ALK-SNX17 | ALK | SNX17 |
| ALK-HS1BP3 | ALK | HS1BP3 |
| ALK-CREBBP | ALK | CREBBP |
| ALK-PAQR4 | ALK | PAQR4 |
| ALK-APH1A | ALK | APH1A |

In some embodiments, the ALK-ABCB11 fusion nucleic acid molecule of the disclosure comprises, in the 5′ to 3′ direction, an ABCB11 gene or a portion thereof fused to an ALK gene or a portion thereof. In some embodiments, the ALK-ACTN4 fusion nucleic acid molecule of the disclosure comprises, in the 5′ to 3′ direction, an ACTN4 gene or a portion thereof fused to an ALK gene or a portion thereof. In some embodiments, the ALK-AGAP1 fusion nucleic acid molecule of the disclosure comprises, in the 5′ to 3′ direction, an AGAP1 gene or a portion thereof fused to an ALK gene or a portion thereof. In some embodiments, the ALK-AGAP1 fusion nucleic acid molecule of the disclosure comprises, in the 5′ to 3′ direction, an ALK gene or a portion thereof fused to an AGAP1 gene or a portion thereof. In some embodiments, the ALK-APH1A fusion nucleic acid molecule of the disclosure comprises, in the 5′ to 3′ direction, an ALK gene or a portion thereof fused to an APH1A gene or a portion thereof. In some embodiments, the ALK-AZI2 fusion nucleic acid molecule of the disclosure comprises, in the 5′ to 3′ direction, an AZI2 gene or a portion thereof fused to an ALK gene or a portion thereof. In some embodiments, the ALK-BTBD9 fusion nucleic acid molecule of the disclosure comprises, in the 5′ to 3′ direction, a BTBD9 gene or a portion thereof fused to an ALK gene or a portion thereof. In some embodiments, the ALK-C2orf73 fusion nucleic acid molecule of the disclosure comprises, in the 5′ to 3′ direction, a C2orf73 gene or a portion thereof fused to an ALK gene or a portion thereof. In some embodiments, the ALK-CAPN14 fusion nucleic acid molecule of the disclosure comprises, in the 5′ to 3′ direction, an ALK gene or a portion thereof fused to a CAPN14 gene or a portion thereof. In some embodiments, the ALK-CARMIL1 fusion nucleic acid molecule of the disclosure comprises, in the 5′ to 3′ direction, a CARMIL1 gene or a portion thereof fused to an ALK gene or a portion thereof. In some embodiments, the ALK-CASP8 fusion nucleic acid molecule of the disclosure comprises, in the 5′ to 3′ direction, a CASP8 gene or a portion thereof fused to an ALK gene or a portion thereof. In some embodiments, the ALK-CDC42BPA fusion nucleic acid molecule of the disclosure comprises, in the 5′ to 3′ direction, a CDC42BPA gene or a portion thereof fused to an ALK gene or a portion thereof. In some embodiments, the ALK-CIB4 fusion nucleic acid molecule of the disclosure comprises, in the 5′ to 3′ direction, a CIB4 gene or a portion thereof fused to an ALK gene or a portion thereof. In some embodiments, the ALK-CNTNAP5 fusion nucleic acid molecule of the disclosure comprises, in the 5′ to 3′ direction, a CNTNAP5 gene or a portion thereof fused to an ALK gene or a portion thereof. In some embodiments, the ALK-COL3A1 fusion nucleic acid molecule of the disclosure comprises, in the 5′ to 3′ direction, a COL3A1 gene or a portion thereof fused to an ALK gene or a portion thereof. In some embodiments, the ALK-CPQ fusion nucleic acid molecule of the disclosure comprises, in the 5′ to 3′ direction, a CPQ gene or a portion thereof fused to an ALK gene or a portion thereof. In some embodiments, the ALK-CPSF7 fusion nucleic acid molecule of the disclosure comprises, in the 5′ to 3′ direction, a CPSF7 gene or a portion thereof fused to an ALK gene or a portion thereof. In some embodiments, the ALK-CREBBP fusion nucleic acid molecule of the disclosure comprises, in the 5′ to 3′ direction, an ALK gene or a portion thereof fused to a CREBBP gene or a portion thereof. In some embodiments, the ALK-CTBP1 fusion nucleic acid molecule of the disclosure comprises, in the 5′ to 3′ direction, a CTBP1 gene or a portion thereof fused to an ALK gene or a portion thereof. In some embodiments, the ALK-CTNND1 fusion nucleic acid molecule of the disclosure comprises, in the 5′ to 3′ direction, a CTNND1 gene or a portion thereof fused to an ALK gene or a portion thereof. In some embodiments, the ALK-CYP51A1 fusion nucleic acid molecule of the disclosure comprises, in the 5′ to 3′ direction, a CYP51A1 gene or a portion thereof fused to an ALK gene or a portion thereof. In some embodiments, the ALK-CYS1 fusion nucleic acid molecule of the disclosure comprises, in the 5′ to 3′ direction, a CYS1 gene or a portion thereof fused to an ALK gene or a portion thereof. In some embodiments, the ALK-EPHA2 fusion nucleic acid molecule of the disclosure comprises, in the 5′ to 3′ direction, an EPHA2 gene or a portion thereof fused to an ALK gene or a portion thereof. In some embodiments, the ALK-FHOD3 fusion nucleic acid molecule of the disclosure comprises, in the 5′ to 3′ direction, an ALK gene or a portion thereof fused to a FHOD3 gene or a portion thereof. In some embodiments, the ALK-GMCL1 fusion nucleic acid molecule of the disclosure comprises, in the 5′ to 3′ direction, an ALK gene or a portion thereof fused to a GMCL1 gene or a portion thereof. In some embodiments, the ALK-GPN1 fusion nucleic acid molecule of the disclosure comprises, in the 5′ to 3′ direction, a GPN1 gene or a portion thereof fused to an ALK gene or a portion thereof. In some embodiments, the ALK-GPR113 fusion nucleic acid molecule of the disclosure comprises, in the 5′ to 3′ direction, a GPR113 gene or a portion thereof fused to an ALK gene or a portion thereof. In some embodiments, the ALK-HADHA fusion nucleic acid molecule of the disclosure comprises, in the 5′ to 3′ direction, a HADHA gene or a portion thereof fused to an ALK gene or a portion thereof. In some embodiments, the ALK-HS1BP3 fusion nucleic acid molecule of the disclosure comprises, in the 5′ to 3′ direction, an ALK gene or a portion thereof fused to an HS1BP3 gene or a portion thereof. In some embodiments, the ALK-INTS9 fusion nucleic acid molecule of the disclosure comprises, in the 5′ to 3′ direction, an INTS9 gene or a portion thereof fused to an ALK gene or a portion thereof. In some embodiments, the ALK-ITGA6 fusion nucleic acid molecule of the disclosure comprises, in the 5′ to 3′ direction, an ALK gene or a portion thereof fused to an ITGA6 gene or a portion thereof. In some embodiments, the ALK-KCTD18 fusion nucleic acid molecule of the disclosure comprises, in the 5′ to 3′ direction, an ALK gene or a portion thereof fused to a KCTD18 gene or a portion thereof. In some embodiments, the ALK-KIF5C fusion nucleic acid molecule of the disclosure comprises, in the 5′ to 3′ direction, a KIF5C gene or a portion thereof fused to an ALK gene or a portion thereof. In some embodiments, the ALK-KLC4 fusion nucleic acid molecule of the disclosure comprises, in the 5′ to 3′ direction, a KLC4 gene or a portion thereof fused to an ALK gene or a portion thereof. In some embodiments, the ALK-LINC00535 fusion nucleic acid molecule of the disclosure comprises, in the 5′ to 3′ direction, a LINC00535 gene or a portion thereof fused to an ALK gene or a portion thereof. In some embodiments, the ALK-LRRFIP2 fusion nucleic acid molecule of the disclosure comprises, in the 5′ to 3′ direction, an LRRFIP2 gene or a portion thereof fused to an ALK gene or a portion thereof. In some embodiments, the ALK-MAGOHB fusion nucleic acid molecule of the disclosure comprises, in the 5′ to 3′ direction, a MAGOHB gene or a portion thereof fused to an ALK gene or a portion thereof. In some embodiments, the ALK-MAMDC4 fusion nucleic acid molecule of the disclosure comprises, in the 5′ to 3′ direction, an ALK gene or a portion thereof fused to a MAMDC4 gene or a portion thereof. In some embodiments, the ALK-MANBA fusion nucleic acid molecule of the disclosure comprises, in the 5′ to 3′ direction, a MANBA gene or a portion thereof fused to an ALK gene or a portion thereof. In some embodiments, the ALK-MAP3K9 fusion nucleic acid molecule of the disclosure comprises, in the 5′ to 3′ direction, an ALK gene or a portion thereof fused to a MAP3K9 gene or a portion thereof. In some embodiments, the ALK-MED13L fusion nucleic acid molecule of the disclosure comprises, in the 5′ to 3′ direction, an ALK gene or a portion thereof fused to an MED13L gene or a portion thereof. In some embodiments, the ALK-METTL25 fusion nucleic acid molecule of the disclosure comprises, in the 5′ to 3′ direction, a METTL25 gene or a portion thereof fused to an ALK gene or a portion thereof. In some embodiments, the ALK-MTBP fusion nucleic acid molecule of the disclosure comprises, in the 5′ to 3′ direction, an ALK gene or a portion thereof fused to an MTBP gene or a portion thereof. In some embodiments, the ALK-MYH10 fusion nucleic acid molecule of the disclosure comprises, in the 5′ to 3′ direction, an MYH10 gene or a portion thereof fused to an ALK gene or a portion thereof. In some embodiments, the ALK-MYO5C fusion nucleic acid molecule of the disclosure comprises, in the 5′ to 3′ direction, a MYO5C gene or a portion thereof fused to an ALK gene or a portion thereof. In some embodiments, the ALK-NFIA fusion nucleic acid molecule of the disclosure comprises, in the 5′ to 3′ direction, an NFIA gene or a portion thereof fused to an ALK gene or a portion thereof. In some embodiments, the ALK-NINJ2 fusion nucleic acid molecule of the disclosure comprises, in the 5′ to 3′ direction, a NINJ2 gene or a portion thereof fused to an ALK gene or a portion thereof. In some embodiments, the ALK-OPRM1 fusion nucleic acid molecule of the disclosure comprises, in the 5′ to 3′ direction, an OPRM1 gene or a portion thereof fused to an ALK gene or a portion thereof. In some embodiments, the ALK-OTX1 fusion nucleic acid molecule of the disclosure comprises, in the 5′ to 3′ direction, an ALK gene or a portion thereof fused to an OTX1 gene or a portion thereof. In some embodiments, the ALK-PAQR4 fusion nucleic acid molecule of the disclosure comprises, in the 5′ to 3′ direction, an ALK gene or a portion thereof fused to a PAQR4 gene or a portion thereof. In some embodiments, the ALK-PDCD10 fusion nucleic acid molecule of the disclosure comprises, in the 5′ to 3′ direction, an ALK gene or a portion thereof fused to a PDCD10 gene or a portion thereof. In some embodiments, the ALK-PDE3A fusion nucleic acid molecule of the disclosure comprises, in the 5′ to 3′ direction, a PDE3A gene or a portion thereof fused to an ALK gene or a portion thereof. In some embodiments, the ALK-PELI1 fusion nucleic acid molecule of the disclosure comprises, in the 5′ to 3′ direction, a PELI1 gene or a portion thereof fused to an ALK gene or a portion thereof. In some embodiments, the ALK-PLEC fusion nucleic acid molecule of the disclosure comprises, in the 5′ to 3′ direction, a PLEC gene or a portion thereof fused to an ALK gene or a portion thereof. In some embodiments, the ALK-PTGER4 fusion nucleic acid molecule of the disclosure comprises, in the 5′ to 3′ direction, an ALK gene or a portion thereof fused to a PTGER4 gene or a portion thereof. In some embodiments, the ALK-PTPRJ fusion nucleic acid molecule of the disclosure comprises, in the 5′ to 3′ direction, an ALK gene or a portion thereof fused to a PTPRJ gene or a portion thereof. In some embodiments, the ALK-QKI fusion nucleic acid molecule of the disclosure comprises, in the 5′ to 3′ direction, a QKI gene or a portion thereof fused to an ALK gene or a portion thereof. In some embodiments, the ALK-RPS6KA5 fusion nucleic acid molecule of the disclosure comprises, in the 5′ to 3′ direction, an RPS6KA5 gene or a portion thereof fused to an ALK gene or a portion thereof. In some embodiments, the ALK-SASH1 fusion nucleic acid molecule of the disclosure comprises, in the 5′ to 3′ direction, a SASH1 gene or a portion thereof fused to an ALK gene or a portion thereof. In some embodiments, the ALK-SEC16B fusion nucleic acid molecule of the disclosure comprises, in the 5′ to 3′ direction, a SEC16B gene or a portion thereof fused to an ALK gene or a portion thereof. In some embodiments, the ALK-SKAP1 fusion nucleic acid molecule of the disclosure comprises, in the 5′ to 3′ direction, a SKAP1 gene or a portion thereof fused to an ALK gene or a portion thereof. In some embodiments, the ALK-SLC25A13 fusion nucleic acid molecule of the disclosure comprises, in the 5′ to 3′ direction, an SLC25A13 gene or a portion thereof fused to an ALK gene or a portion thereof. In some embodiments, the ALK-SLC30A6 fusion nucleic acid molecule of the disclosure comprises, in the 5′ to 3′ direction, an ALK gene or a portion thereof fused to an SLC30A6 gene or a portion thereof. In some embodiments, the ALK-SNX17 fusion nucleic acid molecule of the disclosure comprises, in the 5′ to 3′ direction, an ALK gene or a portion thereof fused to an SNX17 gene or a portion thereof. In some embodiments, the ALK-SOX13 fusion nucleic acid molecule of the disclosure comprises, in the 5′ to 3′ direction, a SOX13 gene or a portion thereof fused to an ALK gene or a portion thereof. In some embodiments, the ALK-SRSF7 fusion nucleic acid molecule of the disclosure comprises, in the 5′ to 3′ direction, an SRSF7 gene or a portion thereof fused to an ALK gene or a portion thereof. In some embodiments, the ALK-TANGO6 fusion nucleic acid molecule of the disclosure comprises, in the 5′ to 3′ direction, a TANGO6 gene or a portion thereof fused to an ALK gene or a portion thereof. In some embodiments, the ALK-TG fusion nucleic acid molecule of the disclosure comprises, in the 5′ to 3′ direction, a TG gene or a portion thereof fused to an ALK gene or a portion thereof. In some embodiments, the ALK-TMCO3 fusion nucleic acid molecule of the disclosure comprises, in the 5′ to 3′ direction, a TMCO3 gene or a portion thereof fused to an ALK gene or a portion thereof. In some embodiments, the ALK-TNS3 fusion nucleic acid molecule of the disclosure comprises, in the 5′ to 3′ direction, a TNS3 gene or a portion thereof fused to an ALK gene or a portion thereof. In some embodiments, the ALK-TRIM24 fusion nucleic acid molecule of the disclosure comprises, in the 5′ to 3′ direction, a TRIM24 gene or a portion thereof fused to an ALK gene or a portion thereof. In some embodiments, the ALK-TTC28 fusion nucleic acid molecule of the disclosure comprises, in the 5′ to 3′ direction, a TTC28 gene or a portion thereof fused to an ALK gene or a portion thereof. In some embodiments, the ALK-UBE2L3 fusion nucleic acid molecule of the disclosure comprises, in the 5′ to 3′ direction, a UBE2L3 gene or a portion thereof fused to an ALK gene or a portion thereof. In some embodiments, the ALK-UBE3B fusion nucleic acid molecule of the disclosure comprises, in the 5′ to 3′ direction, a UBE3B gene or a portion thereof fused to an ALK gene or a portion thereof. In some embodiments, the ALK-UTRN fusion nucleic acid molecule of the disclosure comprises, in the 5′ to 3′ direction, a UTRN gene or a portion thereof fused to an ALK gene or a portion thereof. In some embodiments, the ALK-VASP fusion nucleic acid molecule of the disclosure comprises, in the 5′ to 3′ direction, a VASP gene or a portion thereof fused to an ALK gene or a portion thereof. In some embodiments, the ALK-WDR92 fusion nucleic acid molecule of the disclosure comprises, in the 5′ to 3′ direction, a WDR92 gene or a portion thereof fused to an ALK gene or a portion thereof. In some embodiments, the ALK-YPEL5 fusion nucleic acid molecule of the disclosure comprises, in the 5′ to 3′ direction, an YPEL5 gene or a portion thereof fused to an ALK gene or a portion thereof. In some embodiments, the ALK-ZNF446 fusion nucleic acid molecule of the disclosure comprises, in the 5′ to 3′ direction, a ZNF446 gene or a portion thereof fused to an ALK gene or a portion thereof. In some embodiments, the ALK-ZNF454 fusion nucleic acid molecule of the disclosure comprises, in the 5′ to 3′ direction, an ALK gene or a portion thereof fused to a ZNF454 gene or a portion thereof. In some embodiments, the ALK-ZNF513 fusion nucleic acid molecule of the disclosure comprises, in the 5′ to 3′ direction, a ZNF513 gene or a portion thereof fused to an ALK gene or a portion thereof. In some embodiments, the ALK-ZSWIM2 fusion nucleic acid molecule of the disclosure comprises, in the 5′ to 3′ direction, an ALK gene or a portion thereof fused to a ZSWIM2 gene or a portion thereof. In some embodiments, the ALK-FILIP1L fusion nucleic acid molecule of the disclosure comprises, in the 5′ to 3′ direction, an ALK gene or a portion thereof fused to an FILIP1L gene or a portion thereof.

In some embodiments, an ALK fusion nucleic acid molecule of the disclosure comprises or results from a 5′ breakpoint and/or a 3′ breakpoint within the corresponding exons or introns as indicated in Table 3.

TABLE 3
Exonic and intronic breakpoints of exemplary
ALK fusion nucleic acid molecules.

| ALK Fusion Nucleic | | |
|---|---|---|
| Acid Molecule | 5′ DNA Breakpoint | 3′ DNA Breakpoint |
| ALK-COL3A1 | COL3A1 intron 48 | ALK intron 18 |
| ALK-CDC42BPA | CDC42BPA intron 20 | ALK intron 19 |
| ALK-EPHA2 | EPHA2 intron 3 | ALK intron 18 |
| ALK-MY05C | MYO5C intron 30 | ALK intron 19 |
| ALK-TRIM24 | TRIM24 intron 12 | ALK intron 19 |
| ALK-SKAP1 | SKAP1 intron 3 | ALK intron 19 |
| ALK-UBE3B | UBE3B intron 11 | ALK intron 19 |
| ALK-TNS3 | TNS3 intron 25 | ALK intron 19 |
| ALK-C2orf73 | C2orf73 intron 3 | ALK intron 19 |
| ALK-AZI2 | AZI2 intron 7 | ALK intron 19 |
| ALK-MANBA | MANBA intron 14 | ALK intron 19 |
| ALK-CNTNAP5 | CNTNAP5 intron 2 | ALK intron 19 |
| ALK-TANGO6 | TANGO6 intron 1 | ALK intron 1 |
| ALK-NFIA | NFIA intron 2 | ALK intron 1 |
| ALK-RPS6KA5 | RPS6KA5 intron 1 | ALK intron 7 |
| ALK-TG | TG intron 10 | ALK intron 18 |
| ALK-LRRFIP2 | LRRFIP2 intron 25 | ALK intron 19 |
| ALK-MYO5C | MYO5C intron 30 | ALK intron 19 |
| ALK-AGAP1 | AGAP1 intron 6 | ALK intron 17 |
| ALK-MED13L | ALK intron 1 | MED13L intron 5 |
| ALK-MTBP | ALK intron 17 | MTBP intron 11 |
| ALK-SLC30A6 | ALK intron 18 | SLC30A6 intron 3 |
| ALK-GMCL1 | ALK intron 27 | GMCL1 intron 6 |
| ALK-AGAP1 | ALK intron 19 | AGAP1 intron 6 |
| ALK-ZNF454 | ALK intron 19 | ZNF454 intron 4 |
| ALK-TTC28 | TTC28 intron 1 | ALK intron 13 |
| ALK-NINJ2 | NINJ2 intron 1 | ALK intron 13 |
| ALK-UTRN | UTRN intron 72 | ALK intron 18 |
| ALK-ACTN4 | ACTN4 intron 7 | ALK intron 19 |
| ALK-CPSF7 | CPSF7 intron 5 | ALK intron 19 |
| ALK-SLC25A13 | SLC25A13 intron 10 | ALK intron 19 |
| ALK-CTNND1 | CTNND1 intron 16 | ALK intron 19 |
| ALK-KLC4 | KLC4 intron 9 | ALK intron 19 |
| ALK-BTBD9 | BTBD9 intron 5 | ALK intron 19 |
| ALK-CPQ | CPQ intron 7 | ALK intron 19 |
| ALK-KIF5C | KIF5C intron 11 | ALK intron 19 |
| ALK-MAGOHB | MAGOHB intron 1 | ALK intron 19 |
| ALK-COL3A1 | COL3A1 intron 48 | ALK intron 18 |
| ALK-OPRM1 | OPRM1 intron 3 | ALK intron 19 |
| ALK-GPN1 | GPN1 intron 1 | ALK intron 15 |
| ALK-SEC16B | SEC16B intron 13 | ALK intron 13 |
| ALK-UBE2L3 | UBE2L3 intron 1 | ALK intron 17 |
| ALK-METTL25 | METTL25 intron 8 | ALK intron 19 |
| ALK-CYS1 | CYS1 intron 1 | ALK intron 19 |
| ALK-ABCB11 | ABCB11 intron 26 | ALK intron 19 |
| ALK-INTS9 | INTS9 intron 8 | ALK intron 19 |
| ALK-CIB4 | CIB4 intron 3 | ALK intron 19 |
| ALK-WDR92 | WDR92 intron 7 | ALK intron 1 |
| ALK-OTX1 | ALK intron 19 | OTX1 intron 4 |
| ALK-PDCD10 | ALK intron 15 | PDCD10 intron 2 |
| ALK-PTGER4 | ALK intron 19 | PTGER4 intron 2 |
| ALK-PTPRJ | ALK intron 19 | PTPRJ intron 1 |
| ALK-ZSWIM2 | ALK intron 19 | ZSWIM2 intron 5 |
| ALK-FHOD3 | ALK intron 18 | FHOD3 intron 9 |
| ALK-FILIP1L | ALK intron 18 | ILIP1L intron 1 |
| ALK-ITGA6 | ALK intron 19 | ITGA6 intron 1 |
| ALK-KCTD18 | ALK intron 19 | KCTD18 intron 1 |
| ALK-MAMDC4 | ALK intron 20 | MAMDC4 intron 15 |
| ALK-PELI1 | PELI1 intron 1 | ALK intron 19 |
| ALK-LINC00535 | LINC00535 intron 5 | ALK intron 19 |
| ALK-CTBP1 | CTBP1 exon 9 | ALK intron 19 |
| ALK-CARMIL1 | CARMIL1 exon 27 | ALK intron 19 |
| ALK-ZNF513 | ZNF513 exon 1 | ALK exon 1 |
| ALK-TMCO3 | TMCO3 intron 6 | ALK exon 11 |
| ALK-SRSF7 | SRSF7 intron 4 | ALK exon 18 |
| ALK-CASP8 | CASP8 exon 10 | ALK intron 1 |
| ALK-CYP51A1 | CYP51A1 intron 9 | ALK exon 3 |
| ALK-GPR113 | GPR113 intron 1 | ALK exon 19 |
| ALK-HADHA | HADHA intron 5 | ALK exon 5 |
| ALK-LRRFIP2 | LRRFIP2 intron 22 | ALK exon 20 |
| ALK-MYH10 | MYH10 exon 38 | ALK intron 19 |
| ALK-PDE3A | PDE3A intron 10 | ALK exon 8 |
| ALK-PLEC | PLEC exon 9 | ALK intron 19 |
| ALK-QKI | QKI intron 2 | ALK exon 20 |
| ALK-SASH1 | SASH1 exon 14 | ALK intron 17 |
| ALK-SRSF7 | SRSF7 intron 5 | ALK exon 18 |
| ALK-VASP | VASP intron 4 | ALK exon 7 |
| ALK-ZNF446 | ZNF446 exon 7 | ALK intron 19 |
| ALK-SOX13 | SOX13 exon 1 | ALK exon 18 |
| ALK-YPEL5 | YPEL5 intron 1 | ALK exon 18 |
| ALK-CAPN14 | ALK exon 14 | CAPN14 intron 2 |
| ALK-MAP3K9 | ALK intron 1 | MAP3K9 exon 4 |
| ALK-SNX17 | ALK intron 13 | SNX17 exon 3 |
| ALK-HS1BP3 | ALK intron 19 | HS1BP3 exon 4 |
| ALK-CREBBP | ALK intron 3 | CREBBP exon 2 |
| ALK-PAQR4 | ALK intron 3 | PAQR4 exon 1 |
| ALK-APHIA | ALK intron 4 | APH1A exon 4 |

In some embodiments, the ALK-COL3A1 fusion nucleic acid molecule of the disclosure comprises or results from a breakpoint within intron 48 of COL3A1 and/or intron 18 of ALK. In some embodiments, the ALK-CDC42BPA fusion nucleic acid molecule of the disclosure comprises or results from a breakpoint within intron 20 of CDC42BPA and/or intron 19 of ALK. In some embodiments, the ALK-EPHA2 fusion nucleic acid molecule of the disclosure comprises or results from a breakpoint within intron 3 of EPHA2 and/or intron 18 of ALK. In some embodiments, the ALK-MYO5C fusion nucleic acid molecule of the disclosure comprises or results from a breakpoint within intron 30 of MYO5C and/or intron 19 of ALK. In some embodiments, the ALK-TRIM24 fusion nucleic acid molecule of the disclosure comprises or results from a breakpoint within intron 12 of TRIM24 and/or intron 19 of ALK. In some embodiments, the ALK-SKAP1 fusion nucleic acid molecule of the disclosure comprises or results from a breakpoint within intron 3 of SKAP1 and/or intron 19 of ALK. In some embodiments, the ALK-UBE3B fusion nucleic acid molecule of the disclosure comprises or results from a breakpoint within intron 11 of UBE3B and/or intron 19 of ALK. In some embodiments, the ALK-TNS3 fusion nucleic acid molecule of the disclosure comprises or results from a breakpoint within intron 25 of TNS3 and/or intron 19 of ALK. In some embodiments, the ALK-C2orf73 fusion nucleic acid molecule of the disclosure comprises or results from a breakpoint within intron 3 of C2orf73 and/or intron 19 of ALK. In some embodiments, the ALK-AZI2 fusion nucleic acid molecule of the disclosure comprises or results from a breakpoint within intron 7 of AZI2 and/or intron 19 of ALK. In some embodiments, the ALK-MANBA fusion nucleic acid molecule of the disclosure comprises or results from a breakpoint within intron 14 of MANBA and/or intron 19 of ALK. In some embodiments, the ALK-CNTNAP5 fusion nucleic acid molecule of the disclosure comprises or results from a breakpoint within intron 2 of CNTNAP5 and/or intron 19 of ALK. In some embodiments, the ALK-TANGO6 fusion nucleic acid molecule of the disclosure comprises or results from a breakpoint within intron 1 of TANGO6 and/or intron 1 of ALK. In some embodiments, the ALK-NFIA fusion nucleic acid molecule of the disclosure comprises or results from a breakpoint within intron 2 of NFIA and/or intron 1 of ALK. In some embodiments, the ALK-RPS6KA5 fusion nucleic acid molecule of the disclosure comprises or results from a breakpoint within intron 1 of RPS6KA5 and/or intron 7 of ALK. In some embodiments, the ALK-TG fusion nucleic acid molecule of the disclosure comprises or results from a breakpoint within intron 10 of TG and/or intron 18 of ALK. In some embodiments, the ALK-LRRFIP2 fusion nucleic acid molecule of the disclosure comprises or results from a breakpoint within intron 25 of LRRFIP2 and/or intron 19 of ALK. In some embodiments, the ALK-MYO5C fusion nucleic acid molecule of the disclosure comprises or results from a breakpoint within intron 30 of MYO5C and/or intron 19 of ALK. In some embodiments, the ALK-AGAP1 fusion nucleic acid molecule of the disclosure comprises or results from a breakpoint within intron 6 of AGAP1 and/or intron 17 of ALK. In some embodiments, the ALK-MED13L fusion nucleic acid molecule of the disclosure comprises or results from a breakpoint within intron 1 of ALK and/or intron 5 of MED13L. In some embodiments, the ALK-MTBP fusion nucleic acid molecule of the disclosure comprises or results from a breakpoint within intron 17 of ALK and/or intron 11 of MTBP. In some embodiments, the ALK-SLC30A6 fusion nucleic acid molecule of the disclosure comprises or results from a breakpoint within intron 18 of ALK and/or intron 3 of SLC30A6. In some embodiments, the ALK-GMCL1 fusion nucleic acid molecule of the disclosure comprises or results from a breakpoint within intron 27 of ALK and/or intron 6 of GMCL1. In some embodiments, the ALK-AGAP1 fusion nucleic acid molecule of the disclosure comprises or results from a breakpoint within intron 19 of ALK and/or intron 6 of AGAP1. In some embodiments, the ALK-ZNF454 fusion nucleic acid molecule of the disclosure comprises or results from a breakpoint within intron 19 of ALK and/or intron 4 of ZNF454. In some embodiments, the ALK-TTC28 fusion nucleic acid molecule of the disclosure comprises or results from a breakpoint within intron 1 of TTC28 and/or intron 13 of ALK. In some embodiments, the ALK-NINJ2 fusion nucleic acid molecule of the disclosure comprises or results from a breakpoint within intron 1 of NINJ2 and/or intron 13 of ALK. In some embodiments, the ALK-UTRN fusion nucleic acid molecule of the disclosure comprises or results from a breakpoint within intron 72 of UTRN and/or intron 18 of ALK. In some embodiments, the ALK-ACTN4 fusion nucleic acid molecule of the disclosure comprises or results from a breakpoint within intron 7 of ACTN4 and/or intron 19 of ALK. In some embodiments, the ALK-CPSF7 fusion nucleic acid molecule of the disclosure comprises or results from a breakpoint within intron 5 of CPSF7 and/or intron 19 of ALK. In some embodiments, the ALK-SLC25A13 fusion nucleic acid molecule of the disclosure comprises or results from a breakpoint within intron 10 of SLC25A13 and/or intron 19 of ALK. In some embodiments, the ALK-CTNND1 fusion nucleic acid molecule of the disclosure comprises or results from a breakpoint within intron 16 of CTNND1 and/or intron 19 of ALK. In some embodiments, the ALK-KLC4 fusion nucleic acid molecule of the disclosure comprises or results from a breakpoint within intron 9 of KLC4 and/or intron 19 of ALK. In some embodiments, the ALK-BTBD9 fusion nucleic acid molecule of the disclosure comprises or results from a breakpoint within intron 5 of BTBD9 and/or intron 19 of ALK. In some embodiments, the ALK-CPQ fusion nucleic acid molecule of the disclosure comprises or results from a breakpoint within intron 7 of CPQ and/or intron 19 of ALK. In some embodiments, the ALK-KIF5C fusion nucleic acid molecule of the disclosure comprises or results from a breakpoint within intron 11 of KIF5C and/or intron 19 of ALK. In some embodiments, the ALK-MAGOHB fusion nucleic acid molecule of the disclosure comprises or results from a breakpoint within intron 1 of MAGOHB and/or intron 19 of ALK. In some embodiments, the ALK-COL3A1 fusion nucleic acid molecule of the disclosure comprises or results from a breakpoint within intron 48 of COL3A1 and/or intron 18 of ALK. In some embodiments, the ALK-OPRM1 fusion nucleic acid molecule of the disclosure comprises or results from a breakpoint within intron 3 of OPRM1 and/or intron 19 of ALK. In some embodiments, the ALK-GPN1 fusion nucleic acid molecule of the disclosure comprises or results from a breakpoint within intron 1 of GPN1 and/or intron 15 of ALK. In some embodiments, the ALK-SEC16B fusion nucleic acid molecule of the disclosure comprises or results from a breakpoint within intron 13 of SEC16B and/or intron 13 of ALK. In some embodiments, the ALK-UBE2L3 fusion nucleic acid molecule of the disclosure comprises or results from a breakpoint within intron 1 of UBE2L3 and/or intron 17 of ALK. In some embodiments, the ALK-METTL25 fusion nucleic acid molecule of the disclosure comprises or results from a breakpoint within intron 8 of METTL25 and/or intron 19 of ALK. In some embodiments, the ALK-CYS1 fusion nucleic acid molecule of the disclosure comprises or results from a breakpoint within intron 1 of CYS1 and/or intron 19 of ALK. In some embodiments, the ALK-ABCB11 fusion nucleic acid molecule of the disclosure comprises or results from a breakpoint within intron 26 of ABCB11 and/or intron 19 of ALK. In some embodiments, the ALK-INTS9 fusion nucleic acid molecule of the disclosure comprises or results from a breakpoint within intron 8 of INTS9 and/or intron 19 of ALK. In some embodiments, the ALK-CIB4 fusion nucleic acid molecule of the disclosure comprises or results from a breakpoint within intron 3 of CIB4 and/or intron 19 of ALK. In some embodiments, the ALK-WDR92 fusion nucleic acid molecule of the disclosure comprises or results from a breakpoint within intron 7 of WDR92 and/or intron 1 of ALK. In some embodiments, the ALK-OTX1 fusion nucleic acid molecule of the disclosure comprises or results from a breakpoint within intron 19 of ALK and/or intron 4 of OTX1. In some embodiments, the ALK-PDCD10 fusion nucleic acid molecule of the disclosure comprises or results from a breakpoint within intron 15 of ALK and/or intron 2 of PDCD10. In some embodiments, the ALK-PTGER4 fusion nucleic acid molecule of the disclosure comprises or results from a breakpoint within intron 19 of ALK and/or intron 2 of PTGER4. In some embodiments, the ALK-PTPRJ fusion nucleic acid molecule of the disclosure comprises or results from a breakpoint within intron 19 of ALK and/or intron 1 of PTPRJ. In some embodiments, the ALK-ZSWIM2 fusion nucleic acid molecule of the disclosure comprises or results from a breakpoint within intron 19 of ALK and/or intron 5 of ZSWIM2. In some embodiments, the ALK-FHOD3 fusion nucleic acid molecule of the disclosure comprises or results from a breakpoint within intron 18 of ALK and/or intron 9 of FHOD3. In some embodiments, the ALK-FILIP1L fusion nucleic acid molecule of the disclosure comprises or results from a breakpoint within intron 18 of ALK and/or intron 1 of FILIP1L. In some embodiments, the ALK-ITGA6 fusion nucleic acid molecule of the disclosure comprises or results from a breakpoint within intron 19 of ALK and/or intron 1 of ITGA6. In some embodiments, the ALK-KCTD18 fusion nucleic acid molecule of the disclosure comprises or results from a breakpoint within intron 19 of ALK and/or intron 1 of KCTD18. In some embodiments, the ALK-MAMDC4 fusion nucleic acid molecule of the disclosure comprises or results from a breakpoint within intron 20 of ALK and/or intron 15 of MAMDC4. In some embodiments, the ALK-PELI1 fusion nucleic acid molecule of the disclosure comprises or results from a breakpoint within intron 1 of PELI1 and/or intron 19 of ALK. In some embodiments, the ALK-LINC00535 fusion nucleic acid molecule of the disclosure comprises or results from a breakpoint within intron 5 of LINC00535 and/or intron 19 of ALK. In some embodiments, the ALK-CTBP1 fusion nucleic acid molecule of the disclosure comprises or results from a breakpoint within exon 9 of CTBP1 and/or intron 19 of ALK. In some embodiments, the ALK-CARMIL1 fusion nucleic acid molecule of the disclosure comprises or results from a breakpoint within exon 27 of CARMIL1 and/or intron 19 of ALK. In some embodiments, the ALK-ZNF513 fusion nucleic acid molecule of the disclosure comprises or results from a breakpoint within exon 1 of ZNF513 and/or exon 1 of ALK. In some embodiments, the ALK-TMCO3 fusion nucleic acid molecule of the disclosure comprises or results from a breakpoint within intron 6 of TMCO3 and/or exon 11 of ALK. In some embodiments, the ALK-SRSF7 fusion nucleic acid molecule of the disclosure comprises or results from a breakpoint within intron 4 of SRSF7 and/or exon 18 of ALK. In some embodiments, the ALK-CASP8 fusion nucleic acid molecule of the disclosure comprises or results from a breakpoint within exon 10 of CASP8 and/or intron 1 of ALK. In some embodiments, the ALK-CYP51A1 fusion nucleic acid molecule of the disclosure comprises or results from a breakpoint within intron 9 of CYP51A1 and/or exon 3 of ALK. In some embodiments, the ALK-GPR113 fusion nucleic acid molecule of the disclosure comprises or results from a breakpoint within intron 1 of GPR113 and/or exon 19 of ALK. In some embodiments, the ALK-HADHA fusion nucleic acid molecule of the disclosure comprises or results from a breakpoint within intron 5 of HADHA and/or exon 5 of ALK. In some embodiments, the ALK-LRRFIP2 fusion nucleic acid molecule of the disclosure comprises or results from a breakpoint within intron 22 of LRRFIP2 and/or exon 20 of ALK. In some embodiments, the ALK-MYH10 fusion nucleic acid molecule of the disclosure comprises or results from a breakpoint within exon 38 of MYH10 and/or intron 19 of ALK. In some embodiments, the ALK-PDE3A fusion nucleic acid molecule of the disclosure comprises or results from a breakpoint within intron 10 of PDE3A and/or exon 8 of ALK. In some embodiments, the ALK-PLEC fusion nucleic acid molecule of the disclosure comprises or results from a breakpoint within exon 9 of PLEC and/or intron 19 of ALK. In some embodiments, the ALK-QKI fusion nucleic acid molecule of the disclosure comprises or results from a breakpoint within intron 2 of QKI and/or exon 20 of ALK. In some embodiments, the ALK-SASH1 fusion nucleic acid molecule of the disclosure comprises or results from a breakpoint within exon 14 of SASH1 and/or intron 17 of ALK. In some embodiments, the ALK-SRSF7 fusion nucleic acid molecule of the disclosure comprises or results from a breakpoint within intron 5 of SRSF7 and/or exon 18 of ALK. In some embodiments, the ALK-VASP fusion nucleic acid molecule of the disclosure comprises or results from a breakpoint within intron 4 of VASP and/or exon 7 of ALK. In some embodiments, the ALK-ZNF446 fusion nucleic acid molecule of the disclosure comprises or results from a breakpoint within exon 7 of ZNF446 and/or intron 19 of ALK. In some embodiments, the ALK-SOX13 fusion nucleic acid molecule of the disclosure comprises or results from a breakpoint within exon 1 of SOX13 and/or exon 18 ALK. In some embodiments, the ALK-YPEL5 fusion nucleic acid molecule of the disclosure comprises or results from a breakpoint within intron 1 of YPEL5 and/or exon 18 of ALK. In some embodiments, the ALK-CAPN14 fusion nucleic acid molecule of the disclosure comprises or results from a breakpoint within exon 14 of ALK and/or intron 2 of CAPN14. In some embodiments, the ALK-MAP3K9 fusion nucleic acid molecule of the disclosure comprises or results from a breakpoint within intron 1 of ALK and/or exon 4 of MAP3K9. In some embodiments, the ALK-SNX17 fusion nucleic acid molecule of the disclosure comprises or results from a breakpoint within intron 13 of ALK and/or exon 3 of SNX17. In some embodiments, the ALK-HS1BP3 fusion nucleic acid molecule of the disclosure comprises or results from a breakpoint within intron 19 of ALK and/or exon 4 of HS1BP3. In some embodiments, the ALK-CREBBP fusion nucleic acid molecule of the disclosure comprises or results from a breakpoint within intron 3 of ALK and/or exon 2 of CREBBP. In some embodiments, the ALK-PAQR4 fusion nucleic acid molecule of the disclosure comprises or results from a breakpoint within intron 3 of ALK and/or exon 1 of PAQR4. In some embodiments, the ALK-APH1A fusion nucleic acid molecule of the disclosure comprises or results from a breakpoint within intron 4 of ALK and/or exon 4 of APH1A.

In some embodiments, an ALK fusion nucleic acid molecule of the disclosure comprises or results from a 5′ breakpoint and/or a 3′ breakpoint within the corresponding chromosomal coordinates as indicated in Table 4.

TABLE 4
Chromosomal coordinates of breakpoints of exemplary
ALK fusion nucleic acid molecules.

| ALK Fusion Nucleic | | |
|---|---|---|
| Acid Molecule | 5′ DNA Breakpoint | 3′ DNA Breakpoint |
| ALK-COL3A1 | chr2: 189873851-189873891 | chr2: 29448329-29448369 |
| ALK-CDC42BPA | chr1: 227258447-227258641 | chr2: 29447175-29447325 |
| ALK-EPHA2 | chr1: 16473032-16473610 | chr2: 29449267-29449791 |
| ALK-MYO5C | chr15: 52513187-52513409 | chr2: 29447042-29447276 |
| ALK-TRIM24 | chr7: 138259438-138259669 | chr2: 29447383-29447690 |
| ALK-SKAP1 | chr17: 46426149-46426398 | chr2: 29446499-29446749 |
| ALK-UBE3B | chr12: 109937277-109937406 | chr2: 29446869-29447287 |
| ALK-TNS3 | chr7: 47333336-47333376 | chr2: 29446279-29446319 |
| ALK-C2orf73 | chr2: 54571576-54571912 | chr2: 29447055-29447357 |
| ALK-AZI2 | chr3: 28367651-28368004 | chr2: 29446143-29446537 |
| ALK-MANBA | chr4: 103560841-103561084 | chr2: 29447666-29447971 |
| ALK-CNTNAP5 | chr2: 124982770-124983057 | chr2: 29447716-29448231 |
| ALK-TANGO6 | chr16: 68877512-68877552 | chr2: 29940445-29940485 |
| ALK-NFIA | chr1: 61554202-61554242 | chr2: 29917777-29917817 |
| ALK-RPS6KA5 | chr14: 91489071-91489199 | chr2: 29541078-29541335 |
| ALK-TG | chr8: 133902377-133902916 | chr2: 29449335-29449926 |
| ALK-LRRFIP2 | chr3: 37098839-37099184 | chr2: 29447444-29447873 |
| ALK-MYO5C | chr15: 52512435-52512748 | chr2: 29447704-29448298 |
| ALK-AGAP1 | chr2: 236691113-236691343 | chr2: 29449663-29449982 |
| ALK-MED13L | chr2: 30142862-30142902 | chr12: 116457697-116457737 |
| ALK-MTBP | chr2: 29450415-29450642 | chr8: 121498376-121498554 |
| ALK-SLC30A6 | chr2: 29449705-29450046 | chr2: 32400119-32400478 |
| ALK-GMCL1 | chr2: 29420355-29420535 | chr2: 70072839-70073051 |
| ALK-AGAP1 | chr2: 29447436-29447953 | chr2: 236681905-236682235 |
| ALK-ZNF454 | chr2: 29446550-29446728 | chr5: 178380665-178380839 |
| ALK-TTC28 | chr22: 29070004-29070137 | chr2: 29456365-29456651 |
| ALK-NINJ2 | chr12: 758744-759024 | chr2: 29456325-29456573 |
| ALK-UTRN | chr6: 145163819-145164312 | chr2: 29448627-29449031 |
| ALK-ACTN4 | chr19: 39200190-39200558 | chr2: 29446977-29447411 |
| ALK-CPSF7 | chr11: 61186432-61186683 | chr2: 29447932-29448250 |
| ALK-SLC25A13 | chr7: 95814076-95814403 | chr2: 29446496-29446887 |
| ALK-CTNND1 | chr11: 57580664-57580962 | chr2: 29446968-29447253 |
| ALK-KLC4 | chr6: 43039012-43039212 | chr2: 29447007-29447235 |
| ALK-BTBD9 | chr6: 38549536-38549796 | chr2: 29447685-29447981 |
| ALK-CPQ | chr8: 98095364-98095503 | chr2: 29447780-29448006 |
| ALK-KIF5C | chr2: 149821504-149821810 | chr2: 29446775-29447077 |
| ALK-MAGOHB | chr12: 10765965-10765965 | chr2: 29446915-29446915 |
| ALK-COL3A1 | chr2: 189873873-189873913 | chr2: 29448334-29448374 |
| ALK-OPRM1 | chr6: 154466451-154466754 | chr2: 29446794-29447151 |
| ALK-GPN1 | chr2: 27851952-27852701 | chr2: 29451600-29452075 |
| ALK-SEC16B | chr1: 177916828-177917021 | chr2: 29456427-29456627 |
| ALK-UBE2L3 | chr22: 21931222-21931915 | chr2: 29450281-29450692 |
| ALK-METTL25 | chr12: 82843057-82843199 | chr2: 29446633-29446761 |
| ALK-CYS1 | chr2: 10214734-10215222 | chr2: 29447165-29447594 |
| ALK-ABCB11 | chr2: 169782189-169782398 | chr2: 29447182-29447532 |
| ALK-INTS9 | chr8: 28655333-28655587 | chr2: 29447949-29448294 |
| ALK-CIB4 | chr2: 26842893-26843172 | chr2: 29448034-29448163 |
| ALK-WDR92 | chr2: 68361884-68361924 | chr2: 29917846-29917886 |
| ALK-OTX1 | chr2: 29446763-29447082 | chr2: 63282360-63282649 |
| ALK-PDCD10 | chr2: 29455118-29455261 | chr3: 167443055-167443292 |
| ALK-PTGER4 | chr2: 29446495-29446590 | chr5: 40688492-40688617 |
| ALK-PTPRJ | chr2: 29447003-29447320 | chr11: 48037100-48037371 |
| ALK-ZSWIM2 | chr2: 29447843-29448180 | chr2: 187699208-187699458 |
| ALK-FHOD3 | chr2: 29449375-29449493 | chr18: 34195667-34195872 |
| ALK-FILIP1L | chr2: 29449660-29449973 | chr3: 99688248-99688442 |
| ALK-ITGA6 | chr2: 29447349-29447349 | chr2: 173326199-173326199 |
| ALK-KCTD18 | chr2: 29447134-29447442 | chr2: 201373509-201373886 |
| ALK-MAMDC4 | chr2: 29446201-29446434 | chr9: 139751298-139751629 |
| ALK-PELI1 | chr2: 64360629-64360629 | chr2: 29448116-29448116 |
| ALK-LINC00535 | chr8: 94669651-94669972 | chr2: 29447755-29448172 |
| ALK-CTBP1 | chr4: 1206164-1206291 | chr2: 29447285-29447572 |
| ALK-CARMIL1 | chr6: 25550917-25551280 | chr2: 29447579-29447824 |
| ALK-ZNF513 | chr2: 27603440-27603620 | chr2: 30143106-30143448 |
| ALK-TMCO3 | chr13: 114163479-114163962 | chr2: 29497970-29498226 |
| ALK-SRSF7 | chr2: 38975362-38975710 | chr2: 29449531-29449920 |
| ALK-CASP8 | chr2: 202151038-202151249 | chr2: 29969128-29969572 |
| ALK-CYP51A1 | chr7: 91745465-91745693 | chr2: 29917599-29917771 |
| ALK-GPR113 | chr2: 26546664-26546664 | chr2: 29448344-29448344 |
| ALK-HADHA | chr2: 26456000-26456242 | chr2: 29606503-29606657 |
| ALK-LRRFIP2 | chr3: 37111067-37111382 | chr2: 29446062-29446344 |
| ALK-MYH10 | chr17: 8383515-8383800 | chr2: 29446135-29446423 |
| ALK-PDE3A | chr12: 20796733-20796837 | chr2: 29541064-29541218 |
| ALK-PLEC | chr8: 145009233-145009424 | chr2: 29447974-29448217 |
| ALK-QKI | chr6: 163890860-163891168 | chr2: 29446065-29446378 |
| ALK-SASH1 | chr6: 148853694-148853955 | chr2: 29449754-29449930 |
| ALK-SRSF7 | chr2: 38974723-38975203 | chr2: 29449640-29449923 |
| ALK-VASP | chr19: 46024804-46025190 | chr2: 29543503-29543740 |
| ALK-ZNF446 | chr19: 58991381-58991664 | chr2: 29446677-29446960 |
| ALK-SOX13 | chr1: 204042405-204042653 | chr2: 29449452-29449864 |
| ALK-YPEL5 | chr2: 30378123-30378424 | chr2: 29449645-29449918 |
| ALK-CAPN14 | chr2: 29456468-29456549 | chr2: 31426279-31426378 |
| ALK-MAP3K9 | chr2: 30142828-30143036 | chr14: 71216554-71216698 |
| ALK-SNX17 | chr2: 29462409-29462706 | chr2: 27595576-27595761 |
| ALK-HS1BP3 | chr2: 29446985-29447221 | chr2: 20838147-20838335 |
| ALK-CREBBP | chr2: 29797462-29797842 | chr16: 3900331-3900680 |
| ALK-PAQR4 | chr2: 29917567-29917851 | chr16: 3019620-3019700 |
| ALK-APH1A | chr2: 29669577-29669873 | chr1: 150239704-150239811 |

In some embodiments, the ALK-COL3A1 fusion nucleic acid molecule of the disclosure comprises or results from a breakpoint within chromosomal coordinates chr2:189873851-189873891 and/or within chromosomal coordinates chr2:29448329-29448369. In some embodiments, the ALK-CDC42BPA fusion nucleic acid molecule of the disclosure comprises or results from a breakpoint within chromosomal coordinates chr1:227258447-227258641 and/or within chromosomal coordinates chr2:29447175-29447325. In some embodiments, the ALK-EPHA2 fusion nucleic acid molecule of the disclosure comprises or results from a breakpoint within chromosomal coordinates chr1:16473032-16473610 and/or within chromosomal coordinates chr2:29449267-29449791. In some embodiments, the ALK-MYO5C fusion nucleic acid molecule of the disclosure comprises or results from a breakpoint within chromosomal coordinates chr15:52513187-52513409 and/or within chromosomal coordinates chr2:29447042-29447276. In some embodiments, the ALK-TRIM24 fusion nucleic acid molecule of the disclosure comprises or results from a breakpoint within chromosomal coordinates chr7:138259438-138259669 and/or within chromosomal coordinates chr2:29447383-29447690. In some embodiments, the ALK-SKAP1 fusion nucleic acid molecule of the disclosure comprises or results from a breakpoint within chromosomal coordinates chr17:46426149-46426398 and/or within chromosomal coordinates chr2:29446499-29446749. In some embodiments, the ALK-UBE3B fusion nucleic acid molecule of the disclosure comprises or results from a breakpoint within chromosomal coordinates chr12:109937277-109937406 and/or within chromosomal coordinates chr2:29446869-29447287. In some embodiments, the ALK-TNS3 fusion nucleic acid molecule of the disclosure comprises or results from a breakpoint within chromosomal coordinates chr7:47333336-47333376 and/or within chromosomal coordinates chr2:29446279-29446319. In some embodiments, the ALK-C2orf73 fusion nucleic acid molecule of the disclosure comprises or results from a breakpoint within chromosomal coordinates chr2:54571576-54571912 and/or within chromosomal coordinates chr2:29447055-29447357. In some embodiments, the ALK-AZI2 fusion nucleic acid molecule of the disclosure comprises or results from a breakpoint within chromosomal coordinates chr3:28367651-28368004 and/or within chromosomal coordinates chr2:29446143-29446537. In some embodiments, the ALK-MANBA fusion nucleic acid molecule of the disclosure comprises or results from a breakpoint within chromosomal coordinates chr4:103560841-103561084 and/or within chromosomal coordinates chr2:29447666-29447971. In some embodiments, the ALK-CNTNAP5 fusion nucleic acid molecule of the disclosure comprises or results from a breakpoint within chromosomal coordinates chr2:124982770-124983057 and/or within chromosomal coordinates chr2:29447716-29448231. In some embodiments, the ALK-TANGO6 fusion nucleic acid molecule of the disclosure comprises or results from a breakpoint within chromosomal coordinates chr16:68877512-68877552 and/or within chromosomal coordinates chr2:29940445-29940485. In some embodiments, the ALK-NFIA fusion nucleic acid molecule of the disclosure comprises or results from a breakpoint within chromosomal coordinates chr1:61554202-61554242 and/or within chromosomal coordinates chr2:29917777-29917817. In some embodiments, the ALK-RPS6KA5 fusion nucleic acid molecule of the disclosure comprises or results from a breakpoint within chromosomal coordinates chr14:91489071-91489199 and/or within chromosomal coordinates chr2:29541078-29541335. In some embodiments, the ALK-TG fusion nucleic acid molecule of the disclosure comprises or results from a breakpoint within chromosomal coordinates chr8:133902377-133902916 and/or within chromosomal coordinates chr2:29449335-29449926. In some embodiments, the ALK-LRRFIP2 fusion nucleic acid molecule of the disclosure comprises or results from a breakpoint within chromosomal coordinates chr3:37098839-37099184 and/or within chromosomal coordinates chr2:29447444-29447873. In some embodiments, the ALK-MYO5C fusion nucleic acid molecule of the disclosure comprises or results from a breakpoint within chromosomal coordinates chr15:52512435-52512748 and/or within chromosomal coordinates chr2:29447704-29448298. In some embodiments, the ALK-AGAP1 fusion nucleic acid molecule of the disclosure comprises or results from a breakpoint within chromosomal coordinates chr2:236691113-236691343 and/or within chromosomal coordinates chr2:29449663-29449982. In some embodiments, the ALK-MED13L fusion nucleic acid molecule of the disclosure comprises or results from a breakpoint within chromosomal coordinates chr2:30142862-30142902 and/or within chromosomal coordinates chr12:116457697-116457737. In some embodiments, the ALK-MTBP fusion nucleic acid molecule of the disclosure comprises or results from a breakpoint within chromosomal coordinates chr2:29450415-29450642 and/or within chromosomal coordinates chr8:121498376-121498554. In some embodiments, the ALK-SLC30A6 fusion nucleic acid molecule of the disclosure comprises or results from a breakpoint within chromosomal coordinates chr2:29449705-29450046 and/or within chromosomal coordinates chr2:32400119-32400478. In some embodiments, the ALK-GMCL1 fusion nucleic acid molecule of the disclosure comprises or results from a breakpoint within chromosomal coordinates chr2:29420355-29420535 and/or within chromosomal coordinates chr2:70072839-70073051. In some embodiments, the ALK-AGAP1 fusion nucleic acid molecule of the disclosure comprises or results from a breakpoint within chromosomal coordinates chr2:29447436-29447953 and/or within chromosomal coordinates chr2:236681905-236682235. In some embodiments, the ALK-ZNF454 fusion nucleic acid molecule of the disclosure comprises or results from a breakpoint within chromosomal coordinates chr2:29446550-29446728 and/or within chromosomal coordinates chr5:178380665-178380839. In some embodiments, the ALK-TTC28 fusion nucleic acid molecule of the disclosure comprises or results from a breakpoint within chromosomal coordinates chr22:29070004-29070137 and/or within chromosomal coordinates chr2:29456365-29456651. In some embodiments, the ALK-NINJ2 fusion nucleic acid molecule of the disclosure comprises or results from a breakpoint within chromosomal coordinates chr12:758744-759024 and/or within chromosomal coordinates chr2:29456325-29456573. In some embodiments, the ALK-UTRN fusion nucleic acid molecule of the disclosure comprises or results from a breakpoint within chromosomal coordinates chr6:145163819-145164312 and/or within chromosomal coordinates chr2:29448627-29449031. In some embodiments, the ALK-ACTN4 fusion nucleic acid molecule of the disclosure comprises or results from a breakpoint within chromosomal coordinates chr19:39200190-39200558 and/or within chromosomal coordinates chr2:29446977-29447411. In some embodiments, the ALK-CPSF7 fusion nucleic acid molecule of the disclosure comprises or results from a breakpoint within chromosomal coordinates chr11:61186432-61186683 and/or within chromosomal coordinates chr2:29447932-29448250. In some embodiments, the ALK-SLC25A13 fusion nucleic acid molecule of the disclosure comprises or results from a breakpoint within chromosomal coordinates chr7:95814076-95814403 and/or within chromosomal coordinates chr2:29446496-29446887. In some embodiments, the ALK-CTNND1 fusion nucleic acid molecule of the disclosure comprises or results from a breakpoint within chromosomal coordinates chr11:57580664-57580962 and/or within chromosomal coordinates chr2:29446968-29447253. In some embodiments, the ALK-KLC4 fusion nucleic acid molecule of the disclosure comprises or results from a breakpoint within chromosomal coordinates chr6:43039012-43039212 and/or within chromosomal coordinates chr2:29447007-29447235. In some embodiments, the ALK-BTBD9 fusion nucleic acid molecule of the disclosure comprises or results from a breakpoint within chromosomal coordinates chr6:38549536-38549796 and/or within chromosomal coordinates chr2:29447685-29447981. In some embodiments, the ALK-CPQ fusion nucleic acid molecule of the disclosure comprises or results from a breakpoint within chromosomal coordinates chr8:98095364-98095503 and/or within chromosomal coordinates chr2:29447780-29448006. In some embodiments, the ALK-KIF5C fusion nucleic acid molecule of the disclosure comprises or results from a breakpoint within chromosomal coordinates chr2:149821504-149821810 and/or within chromosomal coordinates chr2:29446775-29447077. In some embodiments, the ALK-MAGOHB fusion nucleic acid molecule of the disclosure comprises or results from a breakpoint within chromosomal coordinates chr12:10765965-10765965 and/or within chromosomal coordinates chr2:29446915-29446915. In some embodiments, the ALK-COL3A1 fusion nucleic acid molecule of the disclosure comprises or results from a breakpoint within chromosomal coordinates chr2:189873873-189873913 and/or within chromosomal coordinates chr2:29448334-29448374. In some embodiments, the ALK-OPRM1 fusion nucleic acid molecule of the disclosure comprises or results from a breakpoint within chromosomal coordinates chr6:154466451-154466754 and/or within chromosomal coordinates chr2:29446794-29447151. In some embodiments, the ALK-GPN1 fusion nucleic acid molecule of the disclosure comprises or results from a breakpoint within chromosomal coordinates chr2:27851952-27852701 and/or within chromosomal coordinates chr2:29451600-29452075. In some embodiments, the ALK-SEC16B fusion nucleic acid molecule of the disclosure comprises or results from a breakpoint within chromosomal coordinates chr1:177916828-177917021 and/or within chromosomal coordinates chr2:29456427-29456627. In some embodiments, the ALK-UBE2L3 fusion nucleic acid molecule of the disclosure comprises or results from a breakpoint within chromosomal coordinates chr22:21931222-21931915 and/or within chromosomal coordinates chr2:29450281-29450692. In some embodiments, the ALK-METTL25 fusion nucleic acid molecule of the disclosure comprises or results from a breakpoint within chromosomal coordinates chr12:82843057-82843199 and/or within chromosomal coordinates chr2:29446633-29446761. In some embodiments, the ALK-CYS1 fusion nucleic acid molecule of the disclosure comprises or results from a breakpoint within chromosomal coordinates chr2:10214734-10215222 and/or within chromosomal coordinates chr2:29447165-29447594. In some embodiments, the ALK-ABCB11 fusion nucleic acid molecule of the disclosure comprises or results from a breakpoint within chromosomal coordinates chr2:169782189-169782398 and/or within chromosomal coordinates chr2:29447182-29447532. In some embodiments, the ALK-INTS9 fusion nucleic acid molecule of the disclosure comprises or results from a breakpoint within chromosomal coordinates chr8:28655333-28655587 and/or within chromosomal coordinates chr2:29447949-29448294. In some embodiments, the ALK-CIB4 fusion nucleic acid molecule of the disclosure comprises or results from a breakpoint within chromosomal coordinates chr2:26842893-26843172 and/or within chromosomal coordinates chr2:29448034-29448163. In some embodiments, the ALK-WDR92 fusion nucleic acid molecule of the disclosure comprises or results from a breakpoint within chromosomal coordinates chr2:68361884-68361924 and/or within chromosomal coordinates chr2:29917846-29917886. In some embodiments, the ALK-OTX1 fusion nucleic acid molecule of the disclosure comprises or results from a breakpoint within chromosomal coordinates chr2:29446763-29447082 and/or within chromosomal coordinates chr2:63282360-63282649. In some embodiments, the ALK-PDCD10 fusion nucleic acid molecule of the disclosure comprises or results from a breakpoint within chromosomal coordinates chr2:29455118-29455261 and/or within chromosomal coordinates chr3:167443055-167443292. In some embodiments, the ALK-PTGER4 fusion nucleic acid molecule of the disclosure comprises or results from a breakpoint within chromosomal coordinates chr2:29446495-29446590 and/or within chromosomal coordinates chr5:40688492-40688617. In some embodiments, the ALK-PTPRJ fusion nucleic acid molecule of the disclosure comprises or results from a breakpoint within chromosomal coordinates chr2:29447003-29447320 and/or within chromosomal coordinates chr11:48037100-48037371. In some embodiments, the ALK-ZSWIM2 fusion nucleic acid molecule of the disclosure comprises or results from a breakpoint within chromosomal coordinates chr2:29447843-29448180 and/or within chromosomal coordinates chr2:187699208-187699458. In some embodiments, the ALK-FHOD3 fusion nucleic acid molecule of the disclosure comprises or results from a breakpoint within chromosomal coordinates chr2:29449375-29449493 and/or within chromosomal coordinates chr18:34195667-34195872. In some embodiments, the ALK-FILIP1L fusion nucleic acid molecule of the disclosure comprises or results from a breakpoint within chromosomal coordinates chr2:29449660-29449973 and/or within chromosomal coordinates chr3:99688248-99688442. In some embodiments, the ALK-ITGA6 fusion nucleic acid molecule of the disclosure comprises or results from a breakpoint within chromosomal coordinates chr2:29447349-29447349 and/or within chromosomal coordinates chr2:173326199-173326199. In some embodiments, the ALK-KCTD18 fusion nucleic acid molecule of the disclosure comprises or results from a breakpoint within chromosomal coordinates chr2:29447134-29447442 and/or within chromosomal coordinates chr2:201373509-201373886. In some embodiments, the ALK-MAMDC4 fusion nucleic acid molecule of the disclosure comprises or results from a breakpoint within chromosomal coordinates chr2:29446201-29446434 and/or within chromosomal coordinates chr9:139751298-139751629. In some embodiments, the ALK-PELI1 fusion nucleic acid molecule of the disclosure comprises or results from a breakpoint within chromosomal coordinates chr2:64360629-64360629 and/or within chromosomal coordinates chr2:29448116-29448116. In some embodiments, the ALK-LINC00535 fusion nucleic acid molecule of the disclosure comprises or results from a breakpoint within chromosomal coordinates chr8:94669651-94669972 and/or within chromosomal coordinates chr2:29447755-29448172. In some embodiments, the ALK-CTBP1 fusion nucleic acid molecule of the disclosure comprises or results from a breakpoint within chromosomal coordinates chr4:1206164-1206291 and/or within chromosomal coordinates chr2:29447285-29447572. In some embodiments, the ALK-CARMIL1 fusion nucleic acid molecule of the disclosure comprises or results from a breakpoint within chromosomal coordinates chr6:25550917-25551280 and/or within chromosomal coordinates chr2:29447579-29447824. In some embodiments, the ALK-ZNF513 fusion nucleic acid molecule of the disclosure comprises or results from a breakpoint within chromosomal coordinates chr2:27603440-27603620 and/or within chromosomal coordinates chr2:30143106-30143448. In some embodiments, the ALK-TMCO3 fusion nucleic acid molecule of the disclosure comprises or results from a breakpoint within chromosomal coordinates chr13:114163479-114163962 and/or within chromosomal coordinates chr2:29497970-29498226. In some embodiments, the ALK-SRSF7 fusion nucleic acid molecule of the disclosure comprises or results from a breakpoint within chromosomal coordinates chr2:38975362-38975710 and/or within chromosomal coordinates chr2:29449531-29449920. In some embodiments, the ALK-CASP8 fusion nucleic acid molecule of the disclosure comprises or results from a breakpoint within chromosomal coordinates chr2:202151038-202151249 and/or within chromosomal coordinates chr2:29969128-29969572. In some embodiments, the ALK-CYP51A1 fusion nucleic acid molecule of the disclosure comprises or results from a breakpoint within chromosomal coordinates chr7:91745465-91745693 and/or within chromosomal coordinates chr2:29917599-29917771. In some embodiments, the ALK-GPR113 fusion nucleic acid molecule of the disclosure comprises or results from a breakpoint within chromosomal coordinates chr2:26546664-26546664 and/or within chromosomal coordinates chr2:29448344-29448344. In some embodiments, the ALK-HADHA fusion nucleic acid molecule of the disclosure comprises or results from a breakpoint within chromosomal coordinates chr2:26456000-26456242 and/or within chromosomal coordinates chr2:29606503-29606657. In some embodiments, the ALK-LRRFIP2 fusion nucleic acid molecule of the disclosure comprises or results from a breakpoint within chromosomal coordinates chr3:37111067-37111382 and/or within chromosomal coordinates chr2:29446062-29446344. In some embodiments, the ALK-MYH10 fusion nucleic acid molecule of the disclosure comprises or results from a breakpoint within chromosomal coordinates chr17:8383515-8383800 and/or within chromosomal coordinates chr2:29446135-29446423. In some embodiments, the ALK-PDE3A fusion nucleic acid molecule of the disclosure comprises or results from a breakpoint within chromosomal coordinates chr12:20796733-20796837 and/or within chromosomal coordinates chr2:29541064-29541218. In some embodiments, the ALK-PLEC fusion nucleic acid molecule of the disclosure comprises or results from a breakpoint within chromosomal coordinates chr8:145009233-145009424 and/or within chromosomal coordinates chr2:29447974-29448217. In some embodiments, the ALK-QKI fusion nucleic acid molecule of the disclosure comprises or results from a breakpoint within chromosomal coordinates chr6:163890860-163891168 and/or within chromosomal coordinates chr2:29446065-29446378. In some embodiments, the ALK-SASH1 fusion nucleic acid molecule of the disclosure comprises or results from a breakpoint within chromosomal coordinates chr6:148853694-148853955 and/or within chromosomal coordinates chr2:29449754-29449930. In some embodiments, the ALK-SRSF7 fusion nucleic acid molecule of the disclosure comprises or results from a breakpoint within chromosomal coordinates chr2:38974723-38975203 and/or within chromosomal coordinates chr2:29449640-29449923. In some embodiments, the ALK-VASP fusion nucleic acid molecule of the disclosure comprises or results from a breakpoint within chromosomal coordinates chr19:46024804-46025190 and/or within chromosomal coordinates chr2:29543503-29543740. In some embodiments, the ALK-ZNF446 fusion nucleic acid molecule of the disclosure comprises or results from a breakpoint within chromosomal coordinates chr19:58991381-58991664 and/or within chromosomal coordinates chr2:29446677-29446960. In some embodiments, the ALK-SOX13 fusion nucleic acid molecule of the disclosure comprises or results from a breakpoint within chromosomal coordinates chr1:204042405-204042653 and/or within chromosomal coordinates chr2:29449452-29449864. In some embodiments, the ALK-YPEL5 fusion nucleic acid molecule of the disclosure comprises or results from a breakpoint within chromosomal coordinates chr2:30378123-30378424 and/or within chromosomal coordinates chr2:29449645-29449918. In some embodiments, the ALK-CAPN14 fusion nucleic acid molecule of the disclosure comprises or results from a breakpoint within chromosomal coordinates chr2:29456468-29456549 and/or within chromosomal coordinates chr2:31426279-31426378. In some embodiments, the ALK-MAP3K9 fusion nucleic acid molecule of the disclosure comprises or results from a breakpoint within chromosomal coordinates chr2:30142828-30143036 and/or within chromosomal coordinates chr14:71216554-71216698. In some embodiments, the ALK-SNX17 fusion nucleic acid molecule of the disclosure comprises or results from a breakpoint within chromosomal coordinates chr2:29462409-29462706 and/or within chromosomal coordinates chr2:27595576-27595761. In some embodiments, the ALK-HS1BP3 fusion nucleic acid molecule of the disclosure comprises or results from a breakpoint within chromosomal coordinates chr2:29446985-29447221 and/or within chromosomal coordinates chr2:20838147-20838335. In some embodiments, the ALK-CREBBP fusion nucleic acid molecule of the disclosure comprises or results from a breakpoint within chromosomal coordinates chr2:29797462-29797842 and/or within chromosomal coordinates chr16:3900331-3900680. In some embodiments, the ALK-PAQR4 fusion nucleic acid molecule of the disclosure comprises or results from a breakpoint within chromosomal coordinates chr2:29917567-29917851 and/or within chromosomal coordinates chr16:3019620-3019700. In some embodiments, the ALK-APH1A fusion nucleic acid molecule of the disclosure comprises or results from a breakpoint within chromosomal coordinates chr2:29669577-29669873 and/or within chromosomal coordinates chr1:150239704-150239811.

In some embodiments of any of the ALK fusion nucleic acid molecules provided herein, the chromosomal coordinates corresponding to any of the breakpoints described herein correspond to Homo sapiens (human) genome assembly GRCh37 (hg19).

In some embodiments, an ALK fusion nucleic acid molecule of the disclosure comprises or results from a fusion between a 5′ exon, or a portion thereof, and the corresponding 3′ exon, or a portion thereof, as indicated in Table 5.

TABLE 5
Exons fused in exemplary ALK fusion nucleic acid molecules.

| ALK Fusion Nucleic Acid | | |
|---|---|---|
| Molecule | 5′ Exon | 3′ Exon |
| ALK-COL3A1 | COL3A1 exon 48 | ALK exon 19 |
| ALK-CDC42BPA | CDC42BPA exon 20 | ALK exon 20 |
| ALK-EPHA2 | EPHA2 exon 3 | ALK exon 19 |
| ALK-MYO5C | MYO5C exon 30 | ALK exon 20 |
| ALK-TRIM24 | TRIM24 exon 12 | ALK exon 20 |
| ALK-SKAP1 | SKAP1 exon 3 | ALK exon 20 |
| ALK-UBE3B | UBE3B exon 11 | ALK exon 20 |
| ALK-TNS3 | TNS3 exon 25 | ALK exon 20 |
| ALK-C2orf73 | C2orf73 exon 3 | ALK exon 20 |
| ALK-AZI2 | AZI2 exon 7 | ALK exon 20 |
| ALK-MANBA | MANBA exon 14 | ALK exon 20 |
| ALK-CNTNAP5 | CNTNAP5 exon 2 | ALK exon 20 |
| ALK-TANGO6 | TANGO6 exon 1 | ALK exon 2 |
| ALK-NFIA | NFIA exon 2 | ALK exon 2 |
| ALK-RPS6KA5 | RPS6KA5 exon 1 | ALK exon 8 |
| ALK-TG | TG exon 10 | ALK exon 19 |
| ALK-LRRFIP2 | LRRFIP2 exon 25 | ALK exon 20 |
| ALK-MYO5C | MYO5C exon 30 | ALK exon 20 |
| ALK-AGAP1 | AGAP1 exon 6 | ALK exon 18 |
| ALK-MED13L | ALK exon 1 | MED13L exon 6 |
| ALK-MTBP | ALK exon 17 | MTBP exon 12 |
| ALK-SLC30A6 | ALK exon 18 | SLC30A6 exon 4 |
| ALK-GMCL1 | ALK exon 27 | GMCL1 exon 7 |
| ALK-AGAP1 | ALK exon 19 | AGAP1 exon 7 |
| ALK-ZNF454 | ALK exon 19 | ZNF454 exon 5 |
| ALK-TTC28 | TTC28 exon 1 | ALK exon 14 |
| ALK-NINJ2 | NINJ2 exon 1 | ALK exon 14 |
| ALK-UTRN | UTRN exon 72 | ALK exon 19 |
| ALK-ACTN4 | ACTN4 exon 7 | ALK exon 20 |
| ALK-CPSF7 | CPSF7 exon 5 | ALK exon 20 |
| ALK-SLC25A13 | SLC25A13 exon 10 | ALK exon 20 |
| ALK-CTNND1 | CTNND1 exon 16 | ALK exon 20 |
| ALK-KLC4 | KLC4 exon 9 | ALK exon 20 |
| ALK-BTBD9 | BTBD9 exon 5 | ALK exon 20 |
| ALK-CPQ | CPQ exon 7 | ALK exon 20 |
| ALK-KIF5C | KIF5C exon 11 | ALK exon 20 |
| ALK-MAGOHB | MAGOHB exon 1 | ALK exon 20 |
| ALK-COL3A1 | COL3A1 exon 48 | ALK exon 19 |
| ALK-OPRM1 | OPRM1 exon 3 | ALK exon 20 |
| ALK-GPN1 | GPN1 exon 1 | ALK exon 16 |
| ALK-SEC16B | SEC16B exon 13 | ALK exon 14 |
| ALK-UBE2L3 | UBE2L3 exon 1 | ALK exon 18 |
| ALK-METTL25 | METTL25 exon 8 | ALK exon 20 |
| ALK-CYS1 | CYS1 exon 1 | ALK exon 20 |
| ALK-ABCB11 | ABCB11 exon 26 | ALK exon 20 |
| ALK-INTS9 | INTS9 exon 8 | ALK exon 20 |
| ALK-CIB4 | CIB4 exon 3 | ALK exon 20 |
| ALK-WDR92 | WDR92 exon 7 | ALK exon 2 |
| ALK-OTX1 | ALK exon 19 | OTX1 exon 5 |
| ALK-PDCD10 | ALK exon 15 | PDCD10 exon 3 |
| ALK-PTGER4 | ALK exon 19 | PTGER4 exon 3 |
| ALK-PTPRJ | ALK exon 19 | PTPRJ exon 2 |
| ALK-ZSWIM2 | ALK exon 19 | ZSWIM2 exon 6 |
| ALK-FHOD3 | ALK exon 18 | FHOD3 exon 10 |
| ALK-FILIP1L | ALK exon 18 | FILP1L exon 2 |
| ALK-ITGA6 | ALK exon 19 | ITGA6 exon 2 |
| ALK-KCTD18 | ALK exon 19 | KCTD18 exon 2 |
| ALK-MAMDC4 | ALK exon 20 | MAMDC4 exon 16 |
| ALK-PELI1 | PELI1 exon 1 | ALK exon 20 |
| ALK-LINC00535 | LINC00535 exon 5 | ALK exon 20 |
| ALK-CTBP1 | CTBP1 exon 9 | ALK exon 20 |
| ALK-CARMIL1 | CARMILI exon 27 | ALK exon 20 |
| ALK-ZNF513 | ZNF513 exon 1 | ALK exon 1 |
| ALK-TMCO3 | TMCO3 exon 6 | ALK exon 11 |
| ALK-SRSF7 | SRSF7 exon 4 | ALK exon 18 |
| ALK-CASP8 | CASP8 exon 10 | ALK exon 2 |
| ALK-CYP51A1 | CYP51A1 exon 9 | ALK exon 3 |
| ALK-GPR113 | GPR113 exon 1 | ALK exon 19 |
| ALK-HADHA | HADHA exon 5 | ALK exon 5 |
| ALK-LRRFIP2 | LRRFIP2 exon 22 | ALK exon 20 |
| ALK-MYH10 | MYH10 exon 38 | ALK exon 20 |
| ALK-PDE3A | PDE3A exon 10 | ALK exon 8 |
| ALK-PLEC | PLEC exon 9 | ALK exon 20 |
| ALK-QKI | QKI exon 2 | ALK exon 20 |
| ALK-SASH1 | SASH1 exon 14 | ALK exon 18 |
| ALK-SRSF7 | SRSF7 exon 5 | ALK exon 18 |
| ALK-VASP | VASP exon 4 | ALK exon 7 |
| ALK-ZNF446 | ZNF446 exon 7 | ALK exon 20 |
| ALK-SOX13 | SOX13 exon 1 | ALK exon 18 |
| ALK-YPEL5 | YPEL5 exon 1 | ALK exon 18 |
| ALK-CAPN14 | ALK exon 14 | CAPN14 exon 3 |
| ALK-MAP3K9 | ALK exon 1 | MAP3K9 exon 4 |
| ALK-SNX17 | ALK exon 13 | SNX17 exon 3 |
| ALK-HS1BP3 | ALK exon 19 | HS1BP3 exon 4 |
| ALK-CREBBP | ALK exon 3 | CREBBP exon 2 |
| ALK-PAQR4 | ALK exon 3 | PAQR4 exon 1 |
| ALK-APH1A | ALK exon 4 | APH1A exon 4 |

In some embodiments, the ALK-COL3A1 fusion nucleic acid molecule of the disclosure comprises or results from a fusion, in the 5′ to 3′ direction, of exon 48 of COL3A1, or a portion thereof, fused to exon 19 of ALK, or a portion thereof. In some embodiments, the ALK-CDC42BPA fusion nucleic acid molecule of the disclosure comprises or results from a fusion, in the 5′ to 3′ direction, of exon 20 of CDC42B3PA, or a portion thereof, fused to exon 20 of ALK, or a portion thereof. In some embodiments, the ALK-EPHA2 fusion nucleic acid molecule of the disclosure comprises or results from a fusion, in the 5′ to 3′ direction, of exon 3 of EPHA2, or a portion thereof, fused to exon 19 of ALK, or a portion thereof. In some embodiments, the ALK-MY05C fusion nucleic acid molecule of the disclosure comprises or results from a fusion, in the 5′ to 3′ direction, of exon 30 of MY05C, or a portion thereof, fused to exon 20 of ALK, or a portion thereof. In some embodiments, the ALK-TRIM24 fusion nucleic acid molecule of the disclosure comprises or results from a fusion, in the 5′ to 3′ direction, of exon 12 of TRIM24, or a portion thereof, fused to exon 20 of ALK, or a portion thereof. In some embodiments, the ALK-SKAP1 fusion nucleic acid molecule of the disclosure comprises or results from a fusion, in the 5′ to 3′ direction, of exon 3 of SKAP1, or a portion thereof, fused to exon 20 of ALK, or a portion thereof. In some embodiments, the ALK-UBE3B fusion nucleic acid molecule of the disclosure comprises or results from a fusion, in the 5′ to 3′ direction, of exon 11 of UBE3B, or a portion thereof, fused to exon 20 of ALK, or a portion thereof. In some embodiments, the ALK-TNS3 fusion nucleic acid molecule of the disclosure comprises or results from a fusion, in the 5′ to 3′ direction, of exon 25 of TNS3, or a portion thereof, fused to exon 20 of ALK, or a portion thereof. In some embodiments, the ALK-C2orf73 fusion nucleic acid molecule of the disclosure comprises or results from a fusion, in the 5′ to 3′ direction, of exon 3 of C2orf73, or a portion thereof, fused to exon 20 of ALK, or a portion thereof. In some embodiments, the ALK-AZI2 fusion nucleic acid molecule of the disclosure comprises or results from a fusion, in the 5′ to 3′ direction, of exon 7 of AZI2, or a portion thereof, fused to exon 20 of ALK, or a portion thereof. In some embodiments, the ALK-MANBA fusion nucleic acid molecule of the disclosure comprises or results from a fusion, in the 5′ to 3′ direction, of exon 14 of MANBA, or a portion thereof, fused to exon 20 of ALK, or a portion thereof. In some embodiments, the ALK-CNTNAP5 fusion nucleic acid molecule of the disclosure comprises or results from a fusion, in the 5′ to 3′ direction, of exon 2 of CNTNAP5, or a portion thereof, fused to exon 20 of ALK, or a portion thereof. In some embodiments, the ALK-TANGO6 fusion nucleic acid molecule of the disclosure comprises or results from a fusion, in the 5′ to 3′ direction, of exon 1 of TANGO6, or a portion thereof, fused to exon 2 of ALK, or a portion thereof. In some embodiments, the ALK-NFIA fusion nucleic acid molecule of the disclosure comprises or results from a fusion, in the 5′ to 3′ direction, of exon 2 of NFIA, or a portion thereof, fused to exon 2 of ALK, or a portion thereof. In some embodiments, the ALK-RPS6KA5 fusion nucleic acid molecule of the disclosure comprises or results from a fusion, in the 5′ to 3′ direction, of exon 1 of RPS6KA5, or a portion thereof, fused to exon 8 of ALK, or a portion thereof. In some embodiments, the ALK-TG fusion nucleic acid molecule of the disclosure comprises or results from a fusion, in the 5′ to 3′ direction, of exon 10 of TG, or a portion thereof, fused to exon 19 of ALK, or a portion thereof. In some embodiments, the ALK-LRRFIP2 fusion nucleic acid molecule of the disclosure comprises or results from a fusion, in the 5′ to 3′ direction, of exon 25 of LRRFIP2, or a portion thereof, fused to exon 20 of ALK, or a portion thereof. In some embodiments, the ALK-MYO5C fusion nucleic acid molecule of the disclosure comprises or results from a fusion, in the 5′ to 3′ direction, of exon 30 of MYOSC, or a portion thereof, fused to exon 20 of ALK, or a portion thereof. In some embodiments, the ALK-AGAP1 fusion nucleic acid molecule of the disclosure comprises or results from a fusion, in the 5′ to 3′ direction, of exon 6 of AGAP1, or a portion thereof, fused to exon 18 of ALK, or a portion thereof. In some embodiments, the ALK-MED13L fusion nucleic acid molecule of the disclosure comprises or results from a fusion, in the 5′ to 3′ direction, of exon 1 of ALK, or a portion thereof, fused to exon 6 of MED13L, or a portion thereof. In some embodiments, the ALK-MTBP fusion nucleic acid molecule of the disclosure comprises or results from a fusion, in the 5′ to 3′ direction, of exon 17 of ALK, or a portion thereof, fused to exon 12 of MTBP, or a portion thereof. In some embodiments, the ALK-SLC30A6 fusion nucleic acid molecule of the disclosure comprises or results from a fusion, in the 5′ to 3′ direction, of exon 18 of ALK, or a portion thereof, fused to exon 4 of SLC30A6, or a portion thereof. In some embodiments, the ALK-GMCL1 fusion nucleic acid molecule of the disclosure comprises or results from a fusion, in the 5′ to 3′ direction, of exon 27 of ALK, or a portion thereof, fused to exon 7 of GMCL1, or a portion thereof. In some embodiments, the ALK-AGAP1 fusion nucleic acid molecule of the disclosure comprises or results from a fusion, in the 5′ to 3′ direction, of exon 19 of ALK, or a portion thereof, fused to exon 7 of AGAP1, or a portion thereof. In some embodiments, the ALK-ZNF454 fusion nucleic acid molecule of the disclosure comprises or results from a fusion, in the 5′ to 3′ direction, of exon 19 of ALK, or a portion thereof, fused to exon 5 of ZNF454, or a portion thereof. In some embodiments, the ALK-TTC28 fusion nucleic acid molecule of the disclosure comprises or results from a fusion, in the 5′ to 3′ direction, of exon 1 of TTC28, or a portion thereof, fused to exon 14 of ALK, or a portion thereof. In some embodiments, the ALK-NINJ2 fusion nucleic acid molecule of the disclosure comprises or results from a fusion, in the 5′ to 3′ direction, of exon 1 of NINJ2, or a portion thereof, fused to exon 14 of ALK, or a portion thereof. In some embodiments, the ALK-UTRN fusion nucleic acid molecule of the disclosure comprises or results from a fusion, in the 5′ to 3′ direction, of exon 72 of UTRN, or a portion thereof, fused to exon 19 of ALK, or a portion thereof. In some embodiments, the ALK-ACTN4 fusion nucleic acid molecule of the disclosure comprises or results from a fusion, in the 5′ to 3′ direction, of exon 7 of ACTN4, or a portion thereof, fused to exon 20 of ALK, or a portion thereof. In some embodiments, the ALK-CPSF7 fusion nucleic acid molecule of the disclosure comprises or results from a fusion, in the 5′ to 3′ direction, of exon 5 of CPSF7, or a portion thereof, fused to exon 20 of ALK, or a portion thereof. In some embodiments, the ALK-SLC25A13 fusion nucleic acid molecule of the disclosure comprises or results from a fusion, in the 5′ to 3′ direction, of exon 10 of SLC25A13, or a portion thereof, fused to exon 20 of ALK, or a portion thereof. In some embodiments, the ALK-CTNND1 fusion nucleic acid molecule of the disclosure comprises or results from a fusion, in the 5′ to 3′ direction, of exon 16 of CTNND1, or a portion thereof, fused to exon 20 of ALK, or a portion thereof. In some embodiments, the ALK-KLC4 fusion nucleic acid molecule of the disclosure comprises or results from a fusion, in the 5′ to 3′ direction, of exon 9 of KLC4, or a portion thereof, fused to exon 20 of ALK, or a portion thereof. In some embodiments, the ALK-BTBD9 fusion nucleic acid molecule of the disclosure comprises or results from a fusion, in the 5′ to 3′ direction, of exon 5 of BTBD9, or a portion thereof, fused to exon 20 of ALK, or a portion thereof. In some embodiments, the ALK-CPQ fusion nucleic acid molecule of the disclosure comprises or results from a fusion, in the 5′ to 3′ direction, of exon 7 of CPQ, or a portion thereof, fused to exon 20 of ALK, or a portion thereof. In some embodiments, the ALK-KIFSC fusion nucleic acid molecule of the disclosure comprises or results from a fusion, in the 5′ to 3′ direction, of exon 11 of KIF5C, or a portion thereof, fused to exon 20 of ALK, or a portion thereof. In some embodiments, the ALK-MAGOHB fusion nucleic acid molecule of the disclosure comprises or results from a fusion, in the 5′ to 3′ direction, of exon 1 of MAGOHB, or a portion thereof, fused to exon 20 of ALK, or a portion thereof. In some embodiments, the ALK-COL3A1 fusion nucleic acid molecule of the disclosure comprises or results from a fusion, in the 5′ to 3′ direction, of exon 48 of COL3A1, or a portion thereof, fused to exon 19 of ALK, or a portion thereof. In some embodiments, the ALK-OPRM1 fusion nucleic acid molecule of the disclosure comprises or results from a fusion, in the 5′ to 3′ direction, of exon 3 of OPRM1, or a portion thereof, fused to exon 20 of ALK, or a portion thereof. In some embodiments, the ALK-GPN1 fusion nucleic acid molecule of the disclosure comprises or results from a fusion, in the 5′ to 3′ direction, of exon 1 of GPN1, or a portion thereof, fused to exon 16 of ALK, or a portion thereof. In some embodiments, the ALK-SEC16B fusion nucleic acid molecule of the disclosure comprises or results from a fusion, in the 5′ to 3′ direction, of exon 13 of SEC16B, or a portion thereof, fused to exon 14 of ALK, or a portion thereof. In some embodiments, the ALK-UBE2L3 fusion nucleic acid molecule of the disclosure comprises or results from a fusion, in the 5′ to 3′ direction, of exon 1 of UBE2L3, or a portion thereof, fused to exon 18 of ALK, or a portion thereof. In some embodiments, the ALK-METTL25 fusion nucleic acid molecule of the disclosure comprises or results from a fusion, in the 5′ to 3′ direction, of exon 8 of METTL25, or a portion thereof, fused to exon 20 of ALK, or a portion thereof. In some embodiments, the ALK-CYS1 fusion nucleic acid molecule of the disclosure comprises or results from a fusion, in the 5′ to 3′ direction, of exon 1 of CYS1, or a portion thereof, fused to exon 20 of ALK, or a portion thereof. In some embodiments, the ALK-ABCB11 fusion nucleic acid molecule of the disclosure comprises or results from a fusion, in the 5′ to 3′ direction, of exon 26 of ABCB11, or a portion thereof, fused to exon 20 of ALK, or a portion thereof. In some embodiments, the ALK-INTS9 fusion nucleic acid molecule of the disclosure comprises or results from a fusion, in the 5′ to 3′ direction, of exon 8 of INTS9, or a portion thereof, fused to exon 20 of ALK, or a portion thereof. In some embodiments, the ALK-CIB4 fusion nucleic acid molecule of the disclosure comprises or results from a fusion, in the 5′ to 3′ direction, of exon 3 of CIB4, or a portion thereof, fused to exon 20 of ALK, or a portion thereof. In some embodiments, the ALK-WDR92 fusion nucleic acid molecule of the disclosure comprises or results from a fusion, in the 5′ to 3′ direction, of exon 7 of WDR92, or a portion thereof, fused to exon 2 of ALK, or a portion thereof. In some embodiments, the ALK-OTX1 fusion nucleic acid molecule of the disclosure comprises or results from a fusion, in the 5′ to 3′ direction, of exon 19 of ALK, or a portion thereof, fused to exon 5 of OTX1, or a portion thereof. In some embodiments, the ALK-PDCD10 fusion nucleic acid molecule of the disclosure comprises or results from a fusion, in the 5′ to 3′ direction, of exon 15 of ALK, or a portion thereof, fused to exon 3 of PDCD10, or a portion thereof. In some embodiments, the ALK-PTGER4 fusion nucleic acid molecule of the disclosure comprises or results from a fusion, in the 5′ to 3′ direction, of exon 19 of ALK, or a portion thereof, fused to exon 3 of PTGER4, or a portion thereof. In some embodiments, the ALK-PTPRJ fusion nucleic acid molecule of the disclosure comprises or results from a fusion, in the 5′ to 3′ direction, of exon 19 of ALK, or a portion thereof, fused to exon 2 of PTPRJ, or a portion thereof. In some embodiments, the ALK-ZSWIM2 fusion nucleic acid molecule of the disclosure comprises or results from a fusion, in the 5′ to 3′ direction, of exon 19 of ALK, or a portion thereof, fused to exon 6 of ZSWIM2, or a portion thereof. In some embodiments, the ALK-FHOD3 fusion nucleic acid molecule of the disclosure comprises or results from a fusion, in the 5′ to 3′ direction, of exon 18 of ALK, or a portion thereof, fused to exon 10 of FHOD3, or a portion thereof. In some embodiments, the ALK-FILIP1L fusion nucleic acid molecule of the disclosure comprises or results from a fusion, in the 5′ to 3′ direction, of exon 18 of ALK, or a portion thereof, fused to exon 2 of FILP1L, or a portion thereof. In some embodiments, the ALK-ITGA6 fusion nucleic acid molecule of the disclosure comprises or results from a fusion, in the 5′ to 3′ direction, of exon 19 of ALK, or a portion thereof, fused to exon 2 of ITGA6, or a portion thereof. In some embodiments, the ALK-KCTD18 fusion nucleic acid molecule of the disclosure comprises or results from a fusion, in the 5′ to 3′ direction, of exon 19 of ALK, or a portion thereof, fused to exon 2 of KCTD18, or a portion thereof. In some embodiments, the ALK-MAMDC4 fusion nucleic acid molecule of the disclosure comprises or results from a fusion, in the 5′ to 3′ direction, of exon 20 of ALK, or a portion thereof, fused to exon 16 of MAMDC4, or a portion thereof. In some embodiments, the ALK-PELI1 fusion nucleic acid molecule of the disclosure comprises or results from a fusion, in the 5′ to 3′ direction, of exon 1 of PELI1, or a portion thereof, fused to exon 20 of ALK, or a portion thereof. In some embodiments, the ALK-PELI1 fusion nucleic acid molecule of the disclosure comprises or results from a fusion, in the 5′ to 3′ direction, of the 5′ UTR, or a portion thereof, of PELI1 (e.g., as encoded by exon 1 of PELI1 or a portion thereof), fused to exon 20 of ALK, or a portion thereof. In some embodiments, the ALK-LINC00535 fusion nucleic acid molecule of the disclosure comprises or results from a fusion, in the 5′ to 3′ direction, of exon 5 of LINC00535, or a portion thereof, fused to exon 20 of ALK, or a portion thereof. In some embodiments, the ALK-LINC00535 fusion nucleic acid molecule of the disclosure comprises or results from a fusion, in the 5′ to 3′ direction, of a UTR, or a portion thereof, of LINC00535 (e.g., as encoded by exons 1-5 of LINC00535 or a portion thereof), fused to exon 20 of ALK, or a portion thereof. In some embodiments, the ALK-CTBP1 fusion nucleic acid molecule of the disclosure comprises or results from a fusion, in the 5′ to 3′ direction, of exon 9 of CTBP1, or a portion thereof, fused to exon 20 of ALK, or a portion thereof. In some embodiments, the ALK-CARMIL1 fusion nucleic acid molecule of the disclosure comprises or results from a fusion, in the 5′ to 3′ direction, of exon 27 of CARMIL1, or a portion thereof, fused to exon 20 of ALK, or a portion thereof. In some embodiments, the ALK-ZNF513 fusion nucleic acid molecule of the disclosure comprises or results from a fusion, in the 5′ to 3′ direction, of exon 1 of ZNF513, or a portion thereof, fused to exon 1 of ALK, or a portion thereof. In some embodiments, the ALK-TMCO3 fusion nucleic acid molecule of the disclosure comprises or results from a fusion, in the 5′ to 3′ direction, of exon 6 of TMCO3, or a portion thereof, fused to exon 11 of ALK, or a portion thereof. In some embodiments, the ALK-SRSF7 fusion nucleic acid molecule of the disclosure comprises or results from a fusion, in the 5′ to 3′ direction, of exon 4 of SRSF7, or a portion thereof, fused to exon 18 of ALK, or a portion thereof. In some embodiments, the ALK-CASP8 fusion nucleic acid molecule of the disclosure comprises or results from a fusion, in the 5′ to 3′ direction, of exon 10 of CASP8, or a portion thereof, fused to exon 2 of ALK, or a portion thereof. In some embodiments, the ALK-CYP51A1 fusion nucleic acid molecule of the disclosure comprises or results from a fusion, in the 5′ to 3′ direction, of exon 9 of CYP51A1, or a portion thereof, fused to exon 3 of ALK, or a portion thereof. In some embodiments, the ALK-GPR113 fusion nucleic acid molecule of the disclosure comprises or results from a fusion, in the 5′ to 3′ direction, of exon 1 of GPR113, or a portion thereof, fused to exon 19 of ALK, or a portion thereof. In some embodiments, the ALK-HADHA fusion nucleic acid molecule of the disclosure comprises or results from a fusion, in the 5′ to 3′ direction, of exon 5 of HADHA, or a portion thereof, fused to exon 5 of ALK, or a portion thereof. In some embodiments, the ALK-LRRFIP2 fusion nucleic acid molecule of the disclosure comprises or results from a fusion, in the 5′ to 3′ direction, of exon 22 of LRRFIP2, or a portion thereof, fused to exon 20 of ALK, or a portion thereof. In some embodiments, the ALK-MYH10 fusion nucleic acid molecule of the disclosure comprises or results from a fusion, in the 5′ to 3′ direction, of exon 38 of MYH10, or a portion thereof, fused to exon 20 of ALK, or a portion thereof. In some embodiments, the ALK-PDE3A fusion nucleic acid molecule of the disclosure comprises or results from a fusion, in the 5′ to 3′ direction, of exon 10 of PDE3A, or a portion thereof, fused to exon 8 of ALK, or a portion thereof. In some embodiments, the ALK-PLEC fusion nucleic acid molecule of the disclosure comprises or results from a fusion, in the 5′ to 3′ direction, of exon 9 of PLEC, or a portion thereof, fused to exon 20 of ALK, or a portion thereof. In some embodiments, the ALK-QKI fusion nucleic acid molecule of the disclosure comprises or results from a fusion, in the 5′ to 3′ direction, of exon 2 of QKI, or a portion thereof, fused to exon 20 of ALK, or a portion thereof. In some embodiments, the ALK-SASH1 fusion nucleic acid molecule of the disclosure comprises or results from a fusion, in the 5′ to 3′ direction, of exon 14 of SASH1, or a portion thereof, fused to exon 18 of ALK, or a portion thereof. In some embodiments, the ALK-SRSF7 fusion nucleic acid molecule of the disclosure comprises or results from a fusion, in the 5′ to 3′ direction, of exon 5 of SRSF7, or a portion thereof, fused to exon 18 of ALK, or a portion thereof. In some embodiments, the ALK-VASP fusion nucleic acid molecule of the disclosure comprises or results from a fusion, in the 5′ to 3′ direction, of exon 4 of VASP, or a portion thereof, fused to exon 7 of ALK, or a portion thereof. In some embodiments, the ALK-ZNF446 fusion nucleic acid molecule of the disclosure comprises or results from a fusion, in the 5′ to 3′ direction, of exon 7 of ZNF446, or a portion thereof, fused to exon 20 of ALK, or a portion thereof. In some embodiments, the ALK-SOX13 fusion nucleic acid molecule of the disclosure comprises or results from a fusion, in the 5′ to 3′ direction, of exon 1 of SOX13, or a portion thereof, fused to exon 18 of ALK, or a portion thereof. In some embodiments, the ALK-SOX13 fusion nucleic acid molecule of the disclosure comprises or results from a fusion, in the 5′ to 3′ direction, of the 5′ UTR, or a portion thereof, of SOX13 (e.g., as encoded by exon 1 of SOX13 or a portion thereof), fused to exon 18 of ALK, or a portion thereof. In some embodiments, the ALK-YPEL5 fusion nucleic acid molecule of the disclosure comprises or results from a fusion, in the 5′ to 3′ direction, of exon 1 of YPEL5, or a portion thereof, fused to exon 18 of ALK, or a portion thereof. In some embodiments, the ALK-YPEL5 fusion nucleic acid molecule of the disclosure comprises or results from a fusion, in the 5′ to 3′ direction, of the 5′ UTR, or a portion thereof, of YPEL5 (e.g., as encoded by exon 1 of YPEL5 or a portion thereof), fused to exon 18 of ALK, or a portion thereof. In some embodiments, the ALK-CAPN14 fusion nucleic acid molecule of the disclosure comprises or results from a fusion, in the 5′ to 3′ direction, of exon 14 of ALK, or a portion thereof, fused to exon 3 of CAPN14, or a portion thereof. In some embodiments, the ALK-MAP3K9 fusion nucleic acid molecule of the disclosure comprises or results from a fusion, in the 5′ to 3′ direction, of exon 1 of ALK, or a portion thereof, fused to exon 4 of MAP3K9, or a portion thereof. In some embodiments, the ALK-SNX17 fusion nucleic acid molecule of the disclosure comprises or results from a fusion, in the 5′ to 3′ direction, of exon 13 of ALK, or a portion thereof, fused to exon 3 of SNX17, or a portion thereof. In some embodiments, the ALK-HS1BP3 fusion nucleic acid molecule of the disclosure comprises or results from a fusion, in the 5′ to 3′ direction, of exon 19 of ALK, or a portion thereof, fused to exon 4 of HS1BP3, or a portion thereof. In some embodiments, the ALK-CREBBP fusion nucleic acid molecule of the disclosure comprises or results from a fusion, in the 5′ to 3′ direction, of exon 3 of ALK, or a portion thereof, fused to exon 2 of CREBBP, or a portion thereof. In some embodiments, the ALK-PAQR4 fusion nucleic acid molecule of the disclosure comprises or results from a fusion, in the 5′ to 3′ direction, of exon 3 of ALK, or a portion thereof, fused to exon 1 of PAQR4, or a portion thereof.

In some embodiments, the ALK-APH1A fusion nucleic acid molecule of the disclosure comprises or results from a fusion, in the 5′ to 3′ direction, of exon 4 of ALK, or a portion thereof, fused to exon 4 of APH1A, or a portion thereof.

In some embodiments, an ALK fusion nucleic acid molecule of the disclosure comprises, in the 5′ to 3′ direction, the corresponding exons or portions thereof as listed in Table 6.

TABLE 6
Exons in exemplary ALK fusion nucleic acid molecules.

| ALK Fusion Nucleic | Exons fused, in the 5′ |
|---|---|
| Acid Molecule | to 3′ direction |
| ALK-COL3A1 | Exons 1-47, and exon 48 or a portion |
| | thereof, of COL3A1 fused to exon 19 |
| | or a portion thereof, and exons 20-29, |
| | of ALK. |
| ALK-CDC42BPA | Exons 1-19, and exon 20 or a portion |
| | thereof, of CDC42BPA fused to |
| | exon 20 or a portion thereof, and |
| | exons 21-29, of ALK. |
| ALK-EPHA2 | Exons 1-2, and exon 3 or a portion |
| | thereof, of EPHA2 fused to exon 19 |
| | or a portion thereof, and exons 20-29, |
| | of ALK. |
| ALK-MYO5C | Exons 1-29, and exon 30 or a |
| | portion thereof, of MYO5C fused to |
| | exon 20 or a portion thereof, and |
| | exons 21-29, of ALK. |
| ALK-TRIM24 | Exons 1-11, and exon 12 or a portion |
| | thereof, of TRIM24 fused to exon 20 |
| | or a portion thereof, and exons 21-29, |
| | of ALK. |
| ALK-SKAP1 | Exons 1-2, and exon 3 or a portion |
| | thereof, of SKAP1 fused to exon 20 |
| | or a portion thereof, and exons 21-29, |
| | of ALK. |
| ALK-UBE3B | Exons 1-10, and exon 11 or a portion |
| | thereof, of UBE3B fused to exon 20 |
| | or a portion thereof, and exons 21-29, |
| | of ALK. |
| ALK-TNS3 | Exons 1-24, and exon 25 or a portion |
| | thereof, of TNS3 fused to exon 20 |
| | or a portion thereof, and exons 21-29, |
| | of ALK. |
| ALK-C2orf73 | Exons 1-2, and exon 3 or a portion |
| | thereof, of C2orf73 fused to exon 20 |
| | or a portion thereof, and exons 21-29, |
| | of ALK. |
| ALK-AZI2 | Exons 1-6, and exon 7 or a portion |
| | thereof, of AZI2 fused to exon 20 |
| | or a portion thereof, and exons 21-29, |
| | of ALK. |
| ALK-MANBA | Exons 1-13, and exon 14 or a |
| | portion thereof, of MANBA fused |
| | to exon 20 or a portion thereof, and |
| | exons 21-29, of ALK. |
| ALK-CNTNAP5 | Exon 1, and exon 2 or a portion |
| | thereof, of CNTNAP5 fused to exon |
| | 20 or a portion thereof, and exons |
| | 21-29, of ALK. |
| ALK-TANGO6 | Exon 1 or a portion thereof of |
| | TANGO6 fused to exon 2 or a |
| | portion thereof, and exons 3-29, of |
| | ALK. |
| ALK-NFIA | Exon 1, and exon 2 or a portion |
| | thereof, of NFIA fused to exon 2 |
| | or a portion thereof, and exons 3-29, |
| | of ALK. |
| ALK-RPS6KA5 | Exon 1 or a portion thereof of |
| | RPS6KA5 fused to exon 8 or a |
| | portion thereof, and exons 9-29, |
| | of ALK. |
| ALK-TG | Exons 1-9, and exon 10 or a |
| | portion thereof, of TG fused to exon |
| | 19 or a portion thereof, and exons |
| | 20-29, of ALK. |
| ALK-LRRFIP2 | Exons 1-24, and exon 25 or a portion |
| | thereof, of LRRFIP2 fused to exon |
| | 20 or a portion thereof, and exons |
| | 21-29, of ALK. |
| ALK-MYO5C | Exons 1-29, and exon 30 or a |
| | portion thereof, of MYO5C fused to |
| | exon 20 or a portion thereof, and |
| | exons 21-29, of ALK. |
| ALK-AGAP1 | Exons 1-5, and exon 6 or a portion |
| | thereof, of AGAP1 fused to exon 18 |
| | or a portion thereof, and exons 19-29, |
| | of ALK. |
| ALK-MED13L | Exon 1 or a portion thereof of ALK |
| | fused to exon 6 or a portion thereof, |
| | and exons 7-31, of MED13L. |
| ALK-MTBP | Exons 1-16, and exon 17 or a portion |
| | thereof, of ALK fused to exon 12 or |
| | a portion thereof, and exons 13-22, |
| | of MTBP. |
| ALK-SLC30A6 | Exons 1-17, and exon 18 or a |
| | portion thereof, of ALK fused to |
| | exon 4 or a portion thereof, and |
| | exons 5-14, of SLC30A6. |
| ALK-GMCL1 | Exons 1-26, and exon 27 or a portion |
| | thereof, of ALK fused to exon 7 or a |
| | portion thereof, and exons 8-14, of |
| | GMCL1. |
| ALK-AGAP1 | Exons 1-18, and exon 19 or a |
| | portion thereof, of ALK fused to |
| | exon 7 or a portion thereof, and |
| | exons 8-17, of AGAP1. |
| ALK-ZNF454 | Exons 1-18, and exon 19 or a portion |
| | thereof, of ALK fused to exon 5 or |
| | a portion thereof of ZNF454. |
| ALK-TTC28 | Exon 1 or a portion thereof of |
| | TTC28 fused to exon 14 or a portion |
| | thereof, and exons 15-29, of ALK. |
| ALK-NINJ2 | Exon 1 or a portion thereof of NINJ2 |
| | fused to exon 14 or a portion thereof, |
| | and exons 15-29, of ALK. |
| ALK-UTRN | Exons 1-71, and exon 72 or a portion |
| | thereof, of UTRN fused to exon 19 |
| | or a portion thereof, and exons 20-29, |
| | of ALK. |
| ALK-ACTN4 | Exons 1-6, and exon 7 or a portion |
| | thereof, of ACTN4 fused to exon 20 |
| | or a portion thereof, and exons 21-29, |
| | of ALK. |
| ALK-CPSF7 | Exons 1-4, and exon 5 or a portion |
| | thereof, of CPSF7 fused to exon 20 |
| | or a portion thereof, and exons 21-29, |
| | of ALK. |
| ALK-SLC25A13 | Exons 1-9, and exon 10 or a portion |
| | thereof, of SLC25A13 fused to exon |
| | 20 or a portion thereof, and exons |
| | 21-29, of ALK. |
| ALK-CTNND1 | Exons 1-15, and exon 16 or a portion |
| | thereof, of CTNND1 fused to exon |
| | 20 or a portion thereof, and exons |
| | 21-29, of ALK. |
| ALK-KLC4 | Exons 1-8, and exon 9 or a portion |
| | thereof, of KLC4 fused to exon 20 |
| | or a portion thereof, and exons |
| | 21-29, of ALK. |
| ALK-BTBD9 | Exons 1-4, and exon 5 or a portion |
| | thereof, of BTBD9 fused to exon 20 |
| | or a portion thereof, and exons 21-29, |
| | of ALK. |
| ALK-CPQ | Exons 1-6, and exon 7 or a portion |
| | thereof, of CPQ fused to exon 20 or |
| | a portion thereof, and exons 21-29, |
| | of ALK. |
| ALK-KIF5C | Exons 1-10, and exon 11 or a portion |
| | thereof, of KIF5C fused to exon 20 |
| | or a portion thereof, and 21-29, of |
| | ALK. |
| ALK-MAGOHB | Exon 1 or a portion thereof of |
| | MAGOHB fused to exon 20 or a |
| | portion thereof, and exons 21-29, |
| | of ALK. |
| ALK-COL3A1 | Exons 1-47, and exon 48 or a |
| | portion thereof, of COL3A1 fused |
| | to exon 19 or a portion thereof, and |
| | exons 20-29, of ALK. |
| ALK-OPRM1 | Exons 1-2, and exon 3 or a portion |
| | thereof, of OPRM1 fused to exon 20 |
| | or a portion thereof, and exons |
| | 21-29, of ALK. |
| ALK-GPN1 | Exon 1 or a portion thereof of GPN1 |
| | fused to exon 16 or a portion thereof, |
| | and exons 17-29, of ALK. |
| ALK-SEC16B | Exons 1-12, and exon 13 or a |
| | portion thereof, of SEC16B fused to |
| | exon 14 or a portion thereof, and |
| | exons 15-29, of ALK. |
| ALK-UBE2L3 | Exon 1 or a portion thereof of |
| | UBE2L3 fused to exon 18 or a |
| | portion thereof, and exons 19-29, |
| | of ALK. |
| ALK-METTL25 | Exons 1-7, and exon 8 or a portion |
| | thereof, of METTL25 fused to |
| | exon 20 or a portion thereof, and |
| | exons 21-29, of ALK. |
| ALK-CYS1 | Exon 1 or a portion thereof of |
| | CYS1 fused to exon 20 or a |
| | portion thereof, and exons 21-29, |
| | of ALK. |
| ALK-ABCB11 | Exons 1-25, and exon 26 or a |
| | portion thereof, of ABCB11 fused |
| | to exon 20 or a portion thereof, and |
| | exons 21-29, of ALK. |
| ALK-INTS9 | Exons 1-7, and exon 8 or a portion |
| | thereof, of INTS9 fused to exon 20 |
| | or a portion thereof, and exons |
| | 21-29, of ALK. |
| ALK-CIB4 | Exons 1-2, and exon 3 or a portion |
| | thereof, of CIB4 fused to exon 20 |
| | or a portion thereof, and exons 21-29, |
| | of ALK. |
| ALK-WDR92 | Exons 1-6, and exon 7 or a portion |
| | thereof, of WDR92 fused to exon 2 |
| | or a portion thereof, and exons 3-29 |
| | of ALK. |
| ALK-OTX1 | Exons 1-18, and exon 19 or a portion |
| | thereof, of ALK fused to exon 5 or a |
| | portion thereof of OTX1. |
| ALK-PDCD10 | Exons 1-14, and exon 15 or a |
| | portion thereof, of ALK fused to |
| | exon 3 or a portion thereof, and |
| | exons 4-9, of PDCD10. |
| ALK-PTGER4 | Exons 1-18, and exon 19 or a |
| | portion thereof, of ALK fused to |
| | exon 3 or a portion thereof of |
| | PTGER4. |
| ALK-PTPRJ | Exons 1-18, and exon 19 or a |
| | portion thereof, of ALK fused to |
| | exon 2 or a portion thereof, and |
| | exons 3-25, of PTPRJ. |
| ALK-ZSWIM2 | Exons 1-18, and exon 19 or a |
| | portion thereof, of ALK fused to |
| | exon 6 or a portion thereof, and |
| | exons 7-9, of ZSWIM2. |
| ALK-FHOD3 | Exons 1-17, and exon 18 or a |
| | portion thereof, of ALK fused to |
| | exon 10 or a portion thereof, and |
| | exons 11-25, of FOHD3. |
| ALK-FILIP1L | Exons 1-17, and exon 18 or a portion |
| | thereof, of ALK fused to exon 2 or a |
| | portion thereof, and exons 3-6, |
| | of FILP1L. |
| ALK-ITGA6 | Exons 1-18, and exon 19 or a |
| | portion thereof, of ALK fused to |
| | exon 2 or a portion thereof, and |
| | exons 3-26, of ITGA6. |
| ALK-KCTD18 | Exons 1-18, and exon 19 or a |
| | portion thereof, of ALK fused to |
| | exon 2 or a portion thereof, and |
| | exons 3-7, of KCTD18. |
| ALK-MAMDC4 | Exons 1-19, and exon 20 or a |
| | portion thereof, of ALK fused to |
| | exon 16 or a portion thereof, and |
| | exons 17-27, of MAMDC4. |
| ALK-PELI1 | Exon 1 or a portion thereof, of PELI1 |
| | fused to exon 20 or a portion thereof, |
| | and exons 21-29, of ALK. |
| ALK-LINC00535 | Exons 1-4, and exon 5 or a portion |
| | thereof, of LINC00535 fused to exon |
| | 20 or a portion thereof, and exons |
| | 21-29, of ALK. |
| ALK-CTBP1 | Exons 1-8, and exon 9 or a portion |
| | thereof, of CTBP1 fused to exon 20 |
| | or a portion thereof, and exons 21-29 |
| | of ALK. |
| ALK-CARMIL1 | Exons 1-26, and exon 27 or a portion |
| | thereof, of CARMIL1 fused to exon |
| | 20 or a portion thereof, and exons |
| | 21-29 of ALK. |
| ALK-ZNF513 | Exon 1 or a portion thereof of |
| | ZNF513 fused to exon 1 or a portion |
| | thereof, and exons 2-29, of ALK. |
| ALK-TMCO3 | Exons 1-5, and exon 6 or a portion |
| | thereof, of TMCO3 fused to exon 11 |
| | or a portion thereof, and exons 12-29, |
| | of ALK. |
| ALK-SRSF7 | Exons 1-3, and exon 4 or a portion |
| | thereof, of SRSF7 fused to exon 18 |
| | or a portion thereof, and exons |
| | 19-29, of ALK. |
| ALK-CASP8 | Exons 1-9, and exon 10 or a portion |
| | thereof, of CASP8 fused to exon 2 |
| | or a portion thereof, and exons 3-29, |
| | of ALK. |
| ALK-CYP51A1 | Exons 1-8, and exon 9 or a portion |
| | thereof, of CYP51A1 fused to exon |
| | 3 or a portion thereof, and exons |
| | 4-29, of ALK. |
| ALK-GPR113 | Exon 1 or a portion thereof of |
| | GPR113 fused to exon 19 or a |
| | portion thereof, and exons 20-29 |
| | of ALK |
| ALK-HADHA | Exons 1-4, and exon 5 or a portion |
| | thereof, of HADHA fused to exon 5 |
| | or a portion thereof, and exons |
| | 6-29, of ALK. |
| ALK-LRRFIP2 | Exons 1-21, and exon 22 or a |
| | portion thereof, of LRRFIP2 fused |
| | to exon 20 or a portion thereof, |
| | and exons 21-29 of ALK. |
| ALK-MYH10 | Exons 1-37, and exon 38 or a |
| | portion thereof, of MYH10 fused |
| | to exon 20 or a portion thereof, |
| | and exons 21-29 of ALK. |
| ALK-PDE3A | Exons 1-9, and exon 10 or a |
| | portion thereof, of PDE3A fused |
| | to exon 8 or a portion thereof, |
| | and exons 9-29, of ALK. |
| ALK-PLEC | Exons 1-8, and exon 9 or a portion |
| | thereof, of PLEC fused to exon 20 |
| | or a portion thereof, and exons 21-29 |
| | of ALK. |
| ALK-QKI | Exon 1, and exon 2 or a portion |
| | thereof, of QKI fused to exon 20 or |
| | a portion thereof, and exons 21-29 |
| | of ALK. |
| ALK-SASH1 | Exons 1-13, and exon 14 or a portion |
| | thereof, of SASH1 fused to exon 18 |
| | or a portion thereof, and exons 19-29, |
| | of ALK. |
| ALK-SRSF7 | Exons 1-4, and exon 5 or a portion |
| | thereof, of SRSF7 fused to exon 18 |
| | or a portion thereof, and exons |
| | 19-29, of ALK. |
| ALK-VASP | Exons 1-3, and exon 4 or a portion |
| | thereof, of VASP fused to exon 7 |
| | or a portion thereof, and exons |
| | 8-29, of ALK. |
| ALK-ZNF446 | Exons 1-6, and exon 7 or a portion |
| | thereof, of ZNF446 fused to exon |
| | 20 or a portion thereof, and exons |
| | 21-29 of ALK. |
| ALK-SOX13 | Exon 1 or a portion thereof of |
| | SOX13 fused to exon 18 or a |
| | portion thereof, and exons 19-29, |
| | of ALK. |
| ALK-YPEL5 | Exon 1 or a portion thereof of |
| | YPEL5 fused to exon 18 or a |
| | portion thereof, and exons 19-29, |
| | of ALK. |
| ALK-CAPN14 | Exons 1-13, and exon 14 or a |
| | portion thereof, of ALK fused to |
| | exon 3 or a portion thereof, and |
| | exons 4-22, of CAPN14. |
| ALK-MAP3K9 | Exon 1 or a portion thereof of ALK |
| | fused to exon 4 or a portion thereof, |
| | and exons 5-13, of MAP3K9. |
| ALK-SNX17 | Exons 1-12, and exon 13 or a portion |
| | thereof, of ALK fused to exon 3 or |
| | a portion thereof, and exons 4-15, |
| | of SNX17. |
| ALK-HS1BP3 | Exon 1-18, and exon 19 or a portion |
| | thereof, of ALK fused to exon 4 or |
| | a portion thereof, and exons 5-7, |
| | of HS1BP3. |
| ALK-CREBBP | Exons 1-2, and exon 3 or a portion |
| | thereof, of ALK fused to exon 2 |
| | or a portion thereof, and exons 3-31, |
| | of CREBBP. |
| ALK-PAQR4 | Exons 1-2, and exon 3 or a portion |
| | thereof, of ALK fused to exon 1 or |
| | a portion thereof, and exons 2-3, |
| | of PAQR4. |
| ALK-APH1A | Exons 1-3, and exon 4 or a |
| | portion thereof, of ALK fused to |
| | exon 4 or a portion thereof, and |
| | exons 5-6, of APH1A. |

In some embodiments, the ALK-COL3A1 fusion nucleic acid molecule of the disclosure comprises, in the 5′ to 3′ direction, exons 1-47, and exon 48 or a portion thereof, of COL3A1 fused to exon 19 or a portion thereof, and exons 20-29, of ALK. In some embodiments, the ALK-CDC42BPA fusion nucleic acid molecule of the disclosure comprises, in the 5′ to 3′ direction, exons 1-19, and exon 20 or a portion thereof, of CDC42BPA fused to exon 20 or a portion thereof, and exons 21-29, of ALK. In some embodiments, the ALK-EPHA2 fusion nucleic acid molecule of the disclosure comprises, in the 5′ to 3′ direction, exons 1-2, and exon 3 or a portion thereof, of EPHA2 fused to exon 19 or a portion thereof, and exons 20-29, of ALK. In some embodiments, the ALK-MYO5C fusion nucleic acid molecule of the disclosure comprises, in the 5′ to 3′ direction, exons 1-29, and exon 30 or a portion thereof, of MYO5C fused to exon 20 or a portion thereof, and exons 21-29, of ALK. In some embodiments, the ALK-TRIM24 fusion nucleic acid molecule of the disclosure comprises, in the 5′ to 3′ direction, exons 1-11, and exon 12 or a portion thereof, of TRIM24 fused to exon 20 or a portion thereof, and exons 21-29, of ALK. In some embodiments, the ALK-SKAP1 fusion nucleic acid molecule of the disclosure comprises, in the 5′ to 3′ direction, exons 1-2, and exon 3 or a portion thereof, of SKAP1 fused to exon 20 or a portion thereof, and exons 21-29, of ALK. In some embodiments, the ALK-UBE3B fusion nucleic acid molecule of the disclosure comprises, in the 5′ to 3′ direction, exons 1-10, and exon 11 or a portion thereof, of UBE3B fused to exon 20 or a portion thereof, and exons 21-29, of ALK. In some embodiments, the ALK-TNS3 fusion nucleic acid molecule of the disclosure comprises, in the 5′ to 3′ direction, exons 1-24, and exon 25 or a portion thereof, of TNS3 fused to exon 20 or a portion thereof, and exons 21-29, of ALK. In some embodiments, the ALK-C2orf73 fusion nucleic acid molecule of the disclosure comprises, in the 5′ to 3′ direction, exons 1-2, and exon 3 or a portion thereof, of C2orf73 fused to exon 20 or a portion thereof, and exons 21-29, of ALK. In some embodiments, the ALK-AZI2 fusion nucleic acid molecule of the disclosure comprises, in the 5′ to 3′ direction, exons 1-6, and exon 7 or a portion thereof, of AZI2 fused to exon 20 or a portion thereof, and exons 21-29, of ALK. In some embodiments, the ALK-MANBA fusion nucleic acid molecule of the disclosure comprises, in the 5′ to 3′ direction, exons 1-13, and exon 14 or a portion thereof, of MANBA fused to exon 20 or a portion thereof, and exons 21-29, of ALK. In some embodiments, the ALK-CNTNAP5 fusion nucleic acid molecule of the disclosure comprises, in the 5′ to 3′ direction, exon 1, and exon 2 or a portion thereof, of CNTNAP5 fused to exon 20 or a portion thereof, and exons 21-29, of ALK. In some embodiments, the ALK-TANGO6 fusion nucleic acid molecule of the disclosure comprises, in the 5′ to 3′ direction, exon 1 or a portion thereof of TANGO6 fused to exon 2 or a portion thereof, and exons 3-29, of ALK. In some embodiments, the ALK-NFIA fusion nucleic acid molecule of the disclosure comprises, in the 5′ to 3′ direction, exon 1, and exon 2 or a portion thereof, of NFIA fused to exon 2 or a portion thereof, and exons 3-29, of ALK. In some embodiments, the ALK-RPS6KA5 fusion nucleic acid molecule of the disclosure comprises, in the 5′ to 3′ direction, exon 1 or a portion thereof of RPS6KA5 fused to exon 8 or a portion thereof, and exons 9-29, of ALK. In some embodiments, the ALK-TG fusion nucleic acid molecule of the disclosure comprises, in the 5′ to 3′ direction, exons 1-9, and exon 10 or a portion thereof, of TG fused to exon 19 or a portion thereof, and exons 20-29, of ALK. In some embodiments, the ALK-LRRFIP2 fusion nucleic acid molecule of the disclosure comprises, in the 5′ to 3′ direction, exons 1-24, and exon 25 or a portion thereof, of LRRFIP2 fused to exon 20 or a portion thereof, and exons 21-29, of ALK. In some embodiments, the ALK-MYO5C fusion nucleic acid molecule of the disclosure comprises, in the 5′ to 3′ direction, exons 1-29, and exon 30 or a portion thereof, of MYO5C fused to exon 20 or a portion thereof, and exons 21-29, of ALK. In some embodiments, the ALK-AGAP1 fusion nucleic acid molecule of the disclosure comprises, in the 5′ to 3′ direction, exons 1-5, and exon 6 or a portion thereof, of AGAP1 fused to exon 18 or a portion thereof, and exons 19-29, of ALK. In some embodiments, the ALK-MED13L fusion nucleic acid molecule of the disclosure comprises, in the 5′ to 3′ direction, exon 1 or a portion thereof of ALK fused to exon 6 or a portion thereof, and exons 7-31, of MED13L. In some embodiments, the ALK-MTBP fusion nucleic acid molecule of the disclosure comprises, in the 5′ to 3′ direction, exons 1-16, and exon 17 or a portion thereof, of ALK fused to exon 12 or a portion thereof, and exons 13-22, of MTBP. In some embodiments, the ALK-SLC30A6 fusion nucleic acid molecule of the disclosure comprises, in the 5′ to 3′ direction, exons 1-17, and exon 18 or a portion thereof, of ALK fused to exon 4 or a portion thereof, and exons 5-14, of SLC30A6. In some embodiments, the ALK-GMCL1 fusion nucleic acid molecule of the disclosure comprises, in the 5′ to 3′ direction, exons 1-26, and exon 27 or a portion thereof, of ALK fused to exon 7 or a portion thereof, and exons 8-14, of GMCL1. In some embodiments, the ALK-AGAP1 fusion nucleic acid molecule of the disclosure comprises, in the 5′ to 3′ direction, exons 1-18, and exon 19 or a portion thereof, of ALK fused to exon 7 or a portion thereof, and exons 8-17, of AGAP1. In some embodiments, the ALK-ZNF454 fusion nucleic acid molecule of the disclosure comprises, in the 5′ to 3′ direction, exons 1-18, and exon 19 or a portion thereof, of ALK fused to exon 5 or a portion thereof of ZNF454. In some embodiments, the ALK-TTC28 fusion nucleic acid molecule of the disclosure comprises, in the 5′ to 3′ direction, exon 1 or a portion thereof of TTC28 fused to exon 14 or a portion thereof, and exons 15-29, of ALK. In some embodiments, the ALK-NINJ2 fusion nucleic acid molecule of the disclosure comprises, in the 5′ to 3′ direction, exon 1 or a portion thereof of NINJ2 fused to exon 14 or a portion thereof, and exons 15-29, of ALK. In some embodiments, the ALK-UTRN fusion nucleic acid molecule of the disclosure comprises, in the 5′ to 3′ direction, exons 1-71, and exon 72 or a portion thereof, of UTRN fused to exon 19 or a portion thereof, and exons 20-29, of ALK. In some embodiments, the ALK-ACTN4 fusion nucleic acid molecule of the disclosure comprises, in the 5′ to 3′ direction, exons 1-6, and exon 7 or a portion thereof, of ACTN4 fused to exon 20 or a portion thereof, and exons 21-29, of ALK. In some embodiments, the ALK-CPSF7 fusion nucleic acid molecule of the disclosure comprises, in the 5′ to 3′ direction, exons 1-4, and exon 5 or a portion thereof, of CPSF7 fused to exon 20 or a portion thereof, and exons 21-29, of ALK. In some embodiments, the ALK-SLC25A13 fusion nucleic acid molecule of the disclosure comprises, in the 5′ to 3′ direction, exons 1-9, and exon 10 or a portion thereof, of SLC25A13 fused to exon 20 or a portion thereof, and exons 21-29, of ALK. In some embodiments, the ALK-CTNND1 fusion nucleic acid molecule of the disclosure comprises, in the 5′ to 3′ direction, exons 1-15, and exon 16 or a portion thereof, of CTNND1 fused to exon 20 or a portion thereof, and exons 21-29, of ALK. In some embodiments, the ALK-KLC4 fusion nucleic acid molecule of the disclosure comprises, in the 5′ to 3′ direction, exons 1-8, and exon 9 or a portion thereof, of KLC4 fused to exon 20 or a portion thereof, and exons 21-29, of ALK. In some embodiments, the ALK-BTBD9 fusion nucleic acid molecule of the disclosure comprises, in the 5′ to 3′ direction, exons 1-4, and exon 5 or a portion thereof, of BTBD9 fused to exon 20 or a portion thereof, and exons 21-29, of ALK. In some embodiments, the ALK-CPQ fusion nucleic acid molecule of the disclosure comprises, in the 5′ to 3′ direction, exons 1-6, and exon 7 or a portion thereof, of CPQ fused to exon 20 or a portion thereof, and exons 21-29, of ALK. In some embodiments, the ALK-KIF5C fusion nucleic acid molecule of the disclosure comprises, in the 5′ to 3′ direction, exons 1-10, and exon 11 or a portion thereof, of KIF5C fused to exon 20 or a portion thereof, and exons 21-29, of ALK. In some embodiments, the ALK-MAGOHB fusion nucleic acid molecule of the disclosure comprises, in the 5′ to 3′ direction, exon 1 or a portion thereof of MAGOHB fused to exon 20 or a portion thereof, and exons 21-29, of ALK. In some embodiments, the ALK-COL3A1 fusion nucleic acid molecule of the disclosure comprises, in the 5′ to 3′ direction, exons 1-47, and exon 48 or a portion thereof, of COL3A1 fused to exon 19 or a portion thereof, and exons 20-29, of ALK. In some embodiments, the ALK-OPRM1 fusion nucleic acid molecule of the disclosure comprises, in the 5′ to 3′ direction, exons 1-2, and exon 3 or a portion thereof, of OPRM1 fused to exon 20 or a portion thereof, and exons 21-29, of ALK. In some embodiments, the ALK-GPN1 fusion nucleic acid molecule of the disclosure comprises, in the 5′ to 3′ direction, exon 1 or a portion thereof of GPN1 fused to exon 16 or a portion thereof, and exons 17-29, of ALK. In some embodiments, the ALK-SEC16B fusion nucleic acid molecule of the disclosure comprises, in the 5′ to 3′ direction, exons 1-12, and exon 13 or a portion thereof, of SEC16B fused to exon 14 or a portion thereof, and exons 15-29, of ALK. In some embodiments, the ALK-UBE2L3 fusion nucleic acid molecule of the disclosure comprises, in the 5′ to 3′ direction, exon 1 or a portion thereof of UBE2L3 fused to exon 18 or a portion thereof, and exons 19-29, of ALK. In some embodiments, the ALK-METTL25 fusion nucleic acid molecule of the disclosure comprises, in the 5′ to 3′ direction, exons 1-7, and exon 8 or a portion thereof, of METTL25 fused to exon 20 or a portion thereof, and exons 21-29, of ALK. In some embodiments, the ALK-CYS1 fusion nucleic acid molecule of the disclosure comprises, in the 5′ to 3′ direction, exon 1 or a portion thereof of CYS1 fused to exon 20 or a portion thereof, and exons 21-29, of ALK. In some embodiments, the ALK-ABCB11 fusion nucleic acid molecule of the disclosure comprises, in the 5′ to 3′ direction, exons 1-25, and exon 26 or a portion thereof, of ABCB11 fused to exon 20 or a portion thereof, and exons 21-29, of ALK. In some embodiments, the ALK-INTS9 fusion nucleic acid molecule of the disclosure comprises, in the 5′ to 3′ direction, exons 1-7, and exon 8 or a portion thereof, of INTS9 fused to exon 20 or a portion thereof, and exons 21-29, of ALK. In some embodiments, the ALK-CIB4 fusion nucleic acid molecule of the disclosure comprises, in the 5′ to 3′ direction, exons 1-2, and exon 3 or a portion thereof, of CIB4 fused to exon 20 or a portion thereof, and exons 21-29, of ALK. In some embodiments, the ALK-WDR92 fusion nucleic acid molecule of the disclosure comprises, in the 5′ to 3′ direction, exons 1-6, and exon 7 or a portion thereof, of WDR92 fused to exon 2 or a portion thereof, and exons 3-29 of ALK. In some embodiments, the ALK-OTX1 fusion nucleic acid molecule of the disclosure comprises, in the 5′ to 3′ direction, exons 1-18, and exon 19 or a portion thereof, of ALK fused to exon 5 or a portion thereof of OTX1. In some embodiments, the ALK-PDCD10 fusion nucleic acid molecule of the disclosure comprises, in the 5′ to 3′ direction, exons 1-14, and exon 15 or a portion thereof, of ALK fused to exon 3 or a portion thereof, and exons 4-9, of PDCD10. In some embodiments, the ALK-PTGER4 fusion nucleic acid molecule of the disclosure comprises, in the 5′ to 3′ direction, exons 1-18, and exon 19 or a portion thereof, of ALK fused to exon 3 or a portion thereof of PTGER4. In some embodiments, the ALK-PTPRJ fusion nucleic acid molecule of the disclosure comprises, in the 5′ to 3′ direction, exons 1-18, and exon 19 or a portion thereof, of ALK fused to exon 2 or a portion thereof, and exons 3-25, of PTPRJ. In some embodiments, the ALK-ZSWIM2 fusion nucleic acid molecule of the disclosure comprises, in the 5′ to 3′ direction, exons 1-18, and exon 19 or a portion thereof, of ALK fused to exon 6 or a portion thereof, and exons 7-9, of ZSWIM2. In some embodiments, the ALK-FHOD3 fusion nucleic acid molecule of the disclosure comprises, in the 5′ to 3′ direction, exons 1-17, and exon 18 or a portion thereof, of ALK fused to exon 10 or a portion thereof, and exons 11-25, of FHOD3. In some embodiments, the ALK-FILIP1L fusion nucleic acid molecule of the disclosure comprises, in the 5′ to 3′ direction, exons 1-17, and exon 18 or a portion thereof, of ALK fused to exon 2 or a portion thereof, and exons 3-6, of FILP1L. In some embodiments, the ALK-ITGA6 fusion nucleic acid molecule of the disclosure comprises, in the 5′ to 3′ direction, exons 1-18, and exon 19 or a portion thereof, of ALK fused to exon 2 or a portion thereof, and exons 3-26, of ITGA6. In some embodiments, the ALK-KCTD18 fusion nucleic acid molecule of the disclosure comprises, in the 5′ to 3′ direction, exons 1-18, and exon 19 or a portion thereof, of ALK fused to exon 2 or a portion thereof, and exons 3-7, of KCTD18. In some embodiments, the ALK-MAMDC4 fusion nucleic acid molecule of the disclosure comprises, in the 5′ to 3′ direction, exons 1-19, and exon 20 or a portion thereof, of ALK fused to exon 16 or a portion thereof, and exons 17-27, of MAMDC4. In some embodiments, the ALK-PELI1 fusion nucleic acid molecule of the disclosure comprises, in the 5′ to 3′ direction, exon 1 or a portion thereof, of PELI1 fused to exon 20 or a portion thereof, and exons 21-29, of ALK. In some embodiments, the ALK-PELI1 fusion nucleic acid molecule of the disclosure comprises, in the 5′ to 3′ direction, the 5′ UTR, or a portion thereof, of PELI1 (e.g., as encoded by exon 1 of PELI1 or a portion thereof), fused to exon 20 or a portion thereof, and exons 21-29, of ALK. In some embodiments, the ALK-LINC00535 fusion nucleic acid molecule of the disclosure comprises, in the 5′ to 3′ direction, exons 1-4, and exon 5 or a portion thereof, of LINC00535 fused to exon 20 or a portion thereof, and exons 21-29, of ALK. In some embodiments, the ALK-LINC00535 fusion nucleic acid molecule of the disclosure comprises, in the 5′ to 3′ direction, a UTR, or a portion thereof, of LINC00535 (e.g., as encoded by exons 1-5 of LINC00535 or a portion thereof), fused to exon 20 or a portion thereof, and exons 21-29, of ALK. In some embodiments, the ALK-CTBP1 fusion nucleic acid molecule of the disclosure comprises, in the 5′ to 3′ direction, exons 1-8, and exon 9 or a portion thereof, of CTBP1 fused to exon 20 or a portion thereof, and exons 21-29 of ALK. In some embodiments, the ALK-CARMIL1 fusion nucleic acid molecule of the disclosure comprises, in the 5′ to 3′ direction, exons 1-26, and exon 27 or a portion thereof, of CARMIL1 fused to exon 20 or a portion thereof, and exons 21-29 of ALK. In some embodiments, the ALK-ZNF513 fusion nucleic acid molecule of the disclosure comprises, in the 5′ to 3′ direction, exon 1 or a portion thereof of ZNF513 fused to exon 1 or a portion thereof, and exons 2-29, of ALK. In some embodiments, the ALK-TMCO3 fusion nucleic acid molecule of the disclosure comprises, in the 5′ to 3′ direction, exons 1-5, and exon 6 or a portion thereof, of TMCO3 fused to exon 11 or a portion thereof, and exons 12-29, of ALK. In some embodiments, the ALK-SRSF7 fusion nucleic acid molecule of the disclosure comprises, in the 5′ to 3′ direction, exons 1-3, and exon 4 or a portion thereof, of SRSF7 fused to exon 18 or a portion thereof, and exons 19-29, of ALK. In some embodiments, the ALK-CASP8 fusion nucleic acid molecule of the disclosure comprises, in the 5′ to 3′ direction, exons 1-9, and exon 10 or a portion thereof, of CASP8 fused to exon 2 or a portion thereof, and exons 3-29, of ALK. In some embodiments, the ALK-CYP51A1 fusion nucleic acid molecule of the disclosure comprises, in the 5′ to 3′ direction, exons 1-8, and exon 9 or a portion thereof, of CYP51A1 fused to exon 3 or a portion thereof, and exons 4-29, of ALK. In some embodiments, the ALK-GPR113 fusion nucleic acid molecule of the disclosure comprises, in the 5′ to 3′ direction, exon 1 or a portion thereof of GPR113 fused to exon 19 or a portion thereof, and exons 20-29 of ALK. In some embodiments, the ALK-HADHA fusion nucleic acid molecule of the disclosure comprises, in the 5′ to 3′ direction, exons 1-4, and exon 5 or a portion thereof, of HADHA fused to exon 5 or a portion thereof, and exons 6-29, of ALK. In some embodiments, the ALK-LRRFIP2 fusion nucleic acid molecule of the disclosure comprises, in the 5′ to 3′ direction, exons 1-21, and exon 22 or a portion thereof, of LRRFIP2 fused to exon 20 or a portion thereof, and exons 21-29 of ALK. In some embodiments, the ALK-MYH10 fusion nucleic acid molecule of the disclosure comprises, in the 5′ to 3′ direction, exons 1-37, and exon 38 or a portion thereof, of MYH10 fused to exon 20 or a portion thereof, and exons 21-29 of ALK. In some embodiments, the ALK-PDE3A fusion nucleic acid molecule of the disclosure comprises, in the 5′ to 3′ direction, exons 1-9, and exon 10 or a portion thereof, of PDE3A fused to exon 8 or a portion thereof, and exons 9-29, of ALK. In some embodiments, the ALK-PLEC fusion nucleic acid molecule of the disclosure comprises, in the 5′ to 3′ direction, exons 1-8, and exon 9 or a portion thereof, of PLEC fused to exon 20 or a portion thereof, and exons 21-29 of ALK. In some embodiments, the ALK-QKI fusion nucleic acid molecule of the disclosure comprises, in the 5′ to 3′ direction, exon 1, and exon 2 or a portion thereof, of QKI fused to exon 20 or a portion thereof, and exons 21-29 of ALK. In some embodiments, the ALK-SASH1 fusion nucleic acid molecule of the disclosure comprises, in the 5′ to 3′ direction, exons 1-13, and exon 14 or a portion thereof, of SASH1 fused to exon 18 or a portion thereof, and exons 19-29, of ALK. In some embodiments, the ALK-SRSF7 fusion nucleic acid molecule of the disclosure comprises, in the 5′ to 3′ direction, exons 1-4, and exon 5 or a portion thereof, of SRSF7 fused to exon 18 or a portion thereof, and exons 19-29, of ALK. In some embodiments, the ALK-VASP fusion nucleic acid molecule of the disclosure comprises, in the 5′ to 3′ direction, exons 1-3, and exon 4 or a portion thereof, of VASP fused to exon 7 or a portion thereof, and exons 8-29, of ALK. In some embodiments, the ALK-ZNF446 fusion nucleic acid molecule of the disclosure comprises, in the 5′ to 3′ direction, exons 1-6, and exon 7 or a portion thereof, of ZNF446 fused to exon 20 or a portion thereof, and exons 21-29 of ALK. In some embodiments, the ALK-SOX13 fusion nucleic acid molecule of the disclosure comprises, in the 5′ to 3′ direction, exon 1 or a portion thereof of SOX13 fused to exon 18 or a portion thereof, and exons 19-29, of ALK. In some embodiments, the ALK-SOX13 fusion nucleic acid molecule of the disclosure comprises, in the 5′ to 3′ direction, the 5′ UTR, or a portion thereof, of SOX13 (e.g., as encoded by exon 1 of SOX13 or a portion thereof), fused to exon 18 or a portion thereof, and exons 19-29, of ALK. In some embodiments, the ALK-YPEL5 fusion nucleic acid molecule of the disclosure comprises, in the 5′ to 3′ direction, exon 1 or a portion thereof of YPEL5 fused to exon 18 or a portion thereof, and exons 19-29, of ALK. In some embodiments, the ALK-YPEL5 fusion nucleic acid molecule of the disclosure comprises, in the 5′ to 3′ direction, the 5′ UTR, or a portion thereof, of YPEL5 (e.g., as encoded by exon 1 of YPEL5 or a portion thereof), fused to exon 18 or a portion thereof, and exons 19-29, of ALK. In some embodiments, the ALK-CAPN14 fusion nucleic acid molecule of the disclosure comprises, in the 5′ to 3′ direction, exons 1-13, and exon 14 or a portion thereof, of ALK fused to exon 3 or a portion thereof, and exons 4-22, of CAPN14. In some embodiments, the ALK-MAP3K9 fusion nucleic acid molecule of the disclosure comprises, in the 5′ to 3′ direction, exon 1 or a portion thereof of ALK fused to exon 4 or a portion thereof, and exons 5-13, of MAP3K9. In some embodiments, the ALK-SNX17 fusion nucleic acid molecule of the disclosure comprises, in the 5′ to 3′ direction, exons 1-12, and exon 13 or a portion thereof, of ALK fused to exon 3 or a portion thereof, and exons 4-15, of SNX17. In some embodiments, the ALK-HS1BP3 fusion nucleic acid molecule of the disclosure comprises, in the 5′ to 3′ direction, exons 1-18, and exon 19 or a portion thereof, of ALK fused to exon 4 or a portion thereof, and exons 5-7, of HS1BP3. In some embodiments, the ALK-CREBBP fusion nucleic acid molecule of the disclosure comprises, in the 5′ to 3′ direction, exons 1-2, and exon 3 or a portion thereof, of ALK fused to exon 2 or a portion thereof, and exons 3-31, of CREBBP. In some embodiments, the ALK-PAQR4 fusion nucleic acid molecule of the disclosure comprises, in the 5′ to 3′ direction, exons 1-2, and exon 3 or a portion thereof, of ALK fused to exon 1 or a portion thereof, and exons 2-3, of PAQR4. In some embodiments, the ALK-APH1A fusion nucleic acid molecule of the disclosure comprises, in the 5′ to 3′ direction, exons 1-3, and exon 4 or a portion thereof, of ALK fused to exon 4 or a portion thereof, and exons 5-6, of APHA.

In some embodiments, an ALK fusion nucleic acid molecule of the disclosure comprises the corresponding nucleotide sequence as listed in Table 7, or a nucleotide sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

TABLE 7
ALK fusion nucleic acid molecule nucleotide sequences.

| ALK | | |
|---|---|---|
| Fusion | | |
| Nucleic | | |
| Acid | | SEQ ID |
| Molecule | ALK Fusion Nucleic Acid Molecule Nucleotide Sequence | NO |
| ALK- | ATGATGAGCTTTGTGCAAAAGGGGAGCTGGCTACTTCTCGCTCTGCTTCATCCCACTA | 1 |
| COL3A1 | TTATTTTGGCACAACAGGAAGCTGTTGAAGGAGGATGTTCCCATCTTGGTCAGTCCTA | |
| | TGCGGATAGAGATGTCTGGAAGCCAGAACCATGCCAAATATGTGTCTGTGACTCAGGA | |
| | TCCGTTCTCTGCGATGACATAATATGTGACGATCAAGAATTAGACTGCCCCAACCCAG | |
| | AAATTCCATTTGGAGAATGTTGTGCAGTTTGCCCACAGCCTCCAACTGCTCCTACTCG | |
| | CCCTCCTAATGGTCAAGGACCTCAAGGCCCCAAGGGAGATCCAGGCCCTCCTGGTATT | |
| | CCTGGGAGAAATGGTGACCCTGGTATTCCAGGACAACCAGGGTCCCCTGGTTCTCCTG | |
| | GCCCCCCTGGAATCTGTGAATCATGCCCTACTGGTCCTCAGAACTATTCTCCCCAGTA | |
| | TGATTCATATGATGTCAAGTCTGGAGTAGCAGTAGGAGGACTCGCAGGCTATCCTGGA | |
| | CCAGCTGGCCCCCCAGGCCCTCCCGGTCCCCCTGGTACATCTGGTCATCCTGGTTCCC | |
| | CTGGATCTCCAGGATACCAAGGACCCCCTGGTGAACCTGGGCAAGCTGGTCCTTCAGG | |
| | CCCTCCAGGACCTCCTGGTGCTATAGGTCCATCTGGTCCTGCTGGAAAAGATGGAGAA | |
| | TCAGGTAGACCCGGACGACCTGGAGAGCGAGGATTGCCTGGACCTCCAGGTATCAAAG | |
| | GTCCAGCTGGGATACCTGGATTCCCTGGTATGAAAGGACACAGAGGCTTCGATGGACG | |
| | AAATGGAGAAAAGGGTGAAACAGGTGCTCCTGGATTAAAGGGTGAAAATGGTCTTCCA | |
| | GGCGAAAATGGAGCTCCTGGACCCATGGGTCCAAGAGGGGCTCCTGGTGAGCGAGGAC | |
| | GGCCAGGACTTCCTGGGGCTGCAGGTGCTCGGGGTAATGACGGTGCTCGAGGCAGTGA | |
| | TGGTCAACCAGGCCCTCCTGGTCCTCCTGGAACTGCCGGATTCCCTGGATCCCCTGGT | |
| | GCTAAGGGTGAAGTTGGACCTGCAGGGTCTCCTGGTTCAAATGGTGCCCCTGGACAAA | |
| | GAGGAGAACCTGGACCTCAGGGACACGCTGGTGCTCAAGGTCCTCCTGGCCCTCCTGG | |
| | GATTAATGGTAGTCCTGGTGGTAAAGGCGAAATGGGTCCCGCTGGCATTCCTGGAGCT | |
| | CCTGGACTGATGGGAGCCCGGGGTCCTCCAGGACCAGCCGGTGCTAATGGTGCTCCTG | |
| | GACTGCGAGGTGGTGCAGGTGAGCCTGGTAAGAATGGTGCCAAAGGAGAGCCCGGACC | |
| | ACGTGGTGAACGCGGTGAGGCTGGTATTCCAGGTGTTCCAGGAGCTAAAGGCGAAGAT | |
| | GGCAAGGATGGATCACCTGGAGAACCTGGTGCAAATGGGCTTCCAGGAGCTGCAGGAG | |
| | AAAGGGGTGCCCCTGGGTTCCGAGGACCTGCTGGACCAAATGGCATCCCAGGAGAAAA | |
| | GGGTCCTGCTGGAGAGCGTGGTGCTCCAGGCCCTGCAGGGCCCAGAGGAGCTGCTGGA | |
| | GAACCTGGCAGAGATGGCGTCCCTGGAGGTCCAGGAATGAGGGGCATGCCCGGAAGTC | |
| | CAGGAGGACCAGGAAGTGATGGGAAACCAGGGCCTCCCGGAAGTCAAGGAGAAAGTGG | |
| | TCGACCAGGTCCTCCTGGGCCATCTGGTCCCCGAGGTCAGCCTGGTGTCATGGGCTTC | |
| | CCCGGTCCTAAAGGAAATGATGGTGCTCCTGGTAAGAATGGAGAACGAGGTGGCCCTG | |
| | GAGGACCTGGCCCTCAGGGTCCTCCTGGAAAGAATGGTGAAACTGGACCTCAGGGACC | |
| | CCCAGGGCCTACTGGGCCTGGTGGTGACAAAGGAGACACAGGACCCCCTGGTCCACAA | |
| | GGATTACAAGGCTTGCCTGGTACAGGTGGTCCTCCAGGAGAAAATGGAAAACCTGGGG | |
| | AACCAGGTCCAAAGGGTGATGCCGGTGCACCTGGAGCTCCAGGAGGCAAGGGTGATGC | |
| | TGGTGCCCCTGGTGAACGTGGACCTCCTGGATTGGCAGGGGCCCCAGGACTTAGAGGT | |
| | GGAGCTGGTCCCCCTGGTCCCGAAGGAGGAAAGGGTGCTGCTGGTCCTCCTGGGCCAC | |
| | CTGGTGCTGCTGGTACTCCTGGTCTGCAAGGAATGCCTGGAGAAAGAGGAGGTCTTGG | |
| | AAGTCCTGGTCCAAAGGGTGACAAGGGTGAACCAGGCGGTCCAGGTGCTGATGGTGTC | |
| | CCAGGGAAAGATGGCCCAAGGGGTCCTACTGGTCCTATTGGTCCTCCTGGCCCAGCTG | |
| | GCCAGCCTGGAGATAAGGGTGAAGGTGGTGCCCCCGGACTTCCAGGTATAGCTGGACC | |
| | TCGTGGTAGCCCTGGTGAGAGAGGTGAAACTGGCCCTCCAGGACCTGCTGGTTTCCCT | |
| | GGTGCTCCTGGACAGAATGGTGAACCTGGTGGTAAAGGAGAAAGAGGGGCTCCGGGTG | |
| | AGAAAGGTGAAGGAGGCCCTCCTGGAGTTGCAGGACCCCCTGGAGGTTCTGGACCTGC | |
| | TGGTCCTCCTGGTCCCCAAGGTGTCAAAGGTGAACGTGGCAGTCCTGGTGGACCTGGT | |
| | GCTGCTGGCTTCCCTGGTGCTCGTGGTCTTCCTGGTCCTCCTGGTAGTAATGGTAACC | |
| | CAGGACCCCCAGGTCCCAGCGGTTCTCCAGGCAAGGATGGGCCCCCAGGTCCTGCGGG | |
| | TAACACTGGTGCTCCTGGCAGCCCTGGAGTGTCTGGACCAAAAGGTGATGCTGGCCAA | |
| | CCAGGAGAGAAGGGATCGCCTGGTGCCCAGGGCCCACCAGGAGCTCCAGGCCCACTTG | |
| | GGATTGCTGGGATCACTGGAGCACGGGGTCTTGCAGGACCACCAGGCATGCCAGGTCC | |
| | TAGGGGAAGCCCTGGCCCTCAGGGTGTCAAGGGTGAAAGTGGGAAACCAGGAGCTAAC | |
| | GGTCTCAGTGGAGAACGTGGTCCCCCTGGACCCCAGGGTCTTCCTGGTCTGGCTGGTA | |
| | CAGCTGGTGAACCTGGAAGAGATGGAAACCCTGGATCAGATGGTCTTCCAGGCCGAGA | |
| | TGGATCTCCTGGTGGCAAGGGTGATCGTGGTGAAAATGGCTCTCCTGGTGCCCCTGGC | |
| | GCTCCTGGTCATCCAGGCCCACCTGGTCCTGTCGGTCCAGCTGGAAAGAGTGGTGACA | |
| | GAGGAGAAAGTGGCCCTGCTGGCCCTGCTGGTGCTCCCGGTCCTGCTGGTTCCCGAGG | |
| | TGCTCCTGGTCCTCAAGGCCCACGTGGTGACAAAGGTGAAACAGGTGAACGTGGAGCT | |
| | GCTGGCATCAAAGGACATCGAGGATTCCCTGGTAATCCAGGTGCCCCAGGTTCTCCAG | |
| | GCCCTGCTGGTCAGCAGGGTGCAATCGGCAGTCCAGGACCTGCAGGCCCCAGAGGACC | |
| | TGTTGGACCCAGTGGACCTCCTGGCAAAGATGGAACCAGTGGACATCCAGGTCCCATT | |
| | GGACCACCAGGGCCTCGAGGTAACAGAGGTGAAAGAGGATCTGAGGGCTCCCCAGGCC | |
| | ACCCAGGGCAACCAGGCCCTCCTGGACCTCCTGGTGCCCCTGGTCCTTGCTGTGGTGG | |
| | TGTTGGAGCCGCTGCCATTGCTGGGATTGGAGGTGAAAAAGCTGGCGGTTTTGCCCCG | |
| | TATTATGGAGATGAACCAATGGATTTCAAAATCAACACCGATGAGATTATGACTTCAC | |
| | TCAAGTCTGTTAATGGACAAATAGAAAGCCTCATTAGTCCTGATGGTTCTCGTAAAAA | |
| | CCCCGCTAGAAACTGCAGAGACCTGAAATTCTGCCATCCTGAACTCAAGAGTGTGTCA | |
| | CCCACCCCGGAGCCACACCTGCCACTCTCGCTGATCCTCTCTGTGGTGACCTCTGCCC | |
| | TCGTGGCCGCCCTGGTCCTGGCTTTCTCCGGCATCATGATTGTGTACCGCCGGAAGCA | |
| | CCAGGAGCTGCAAGCCATGCAGATGGAGCTGCAGAGCCCTGAGTACAAGCTGAGCAAG | |
| | CTCCGCACCTCGACCATCATGACCGACTACAACCCCAACTACTGCTTTGCTGGCAAGA | |
| | CCTCCTCCATCAGTGACCTGAAGGAGGTGCCGCGGAAAAACATCACCCTCATTCGGGG | |
| | TCTGGGCCATGGCGCCTTTGGGGAGGTGTATGAAGGCCAGGTGTCCGGAATGCCCAAC | |
| | GACCCAAGCCCCCTGCAAGTGGCTGTGAAGACGCTGCCTGAAGTGTGCTCTGAACAGG | |
| | ACGAACTGGATTTCCTCATGGAAGCCCTGATCATCAGCAAATTCAACCACCAGAACAT | |
| | TGTTCGCTGCATTGGGGTGAGCCTGCAATCCCTGCCCCGGTTCATCCTGCTGGAGCTC | |
| | ATGGCGGGGGGAGACCTCAAGTCCTTCCTCCGAGAGACCCGCCCTCGCCCGAGCCAGC | |
| | CCTCCTCCCTGGCCATGCTGGACCTTCTGCACGTGGCTCGGGACATTGCCTGTGGCTG | |
| | TCAGTATTTGGAGGAAAACCACTTCATCCACCGAGACATTGCTGCCAGAAACTGCCTC | |
| | TTGACCTGTCCAGGCCCTGGAAGAGTGGCCAAGATTGGAGACTTCGGGATGGCCCGAG | |
| | ACATCTACAGGGCGAGCTACTATAGAAAGGGAGGCTGTGCCATGCTGCCAGTTAAGTG | |
| | GATGCCCCCAGAGGCCTTCATGGAAGGAATATTCACTTCTAAAACAGACACATGGTCC | |
| | TTTGGAGTGCTGCTATGGGAAATCTTTTCTCTTGGATATATGCCATACCCCAGCAAAA | |
| | GCAACCAGGAAGTTCTGGAGTTTGTCACCAGTGGAGGCCGGATGGACCCACCCAAGAA | |
| | CTGCCCTGGGCCTGTATACCGGATAATGACTCAGTGCTGGCAACATCAGCCTGAAGAC | |
| | AGGCCCAACTTTGCCATCATTTTGGAGAGGATTGAATACTGCACCCAGGACCCGGATG | |
| | TAATCAACACCGCTTTGCCGATAGAATATGGTCCACTTGTGGAAGAGGAAGAGAAAGT | |
| | GCCTGTGAGGCCCAAGGACCCTGAGGGGGTTCCTCCTCTCCTGGTCTCTCAACAGGCA | |
| | AAACGGGAGGAGGAGCGCAGCCCAGCTGCCCCACCACCTCTGCCTACCACCTCCTCTG | |
| | GCAAGGCTGCAAAGAAACCCACAGCTGCAGAGATCTCTGTTCGAGTCCCTAGAGGGCC | |
| | GGCCGTGGAAGGGGGACACGTGAATATGGCATTCTCTCAGTCCAACCCTCCTTCGGAG | |
| | TTGCACAAGGTCCACGGATCCAGAAACAAGCCCACCAGCTTGTGGAACCCAACGTACG | |
| | GCTCCTGGTTTACAGAGAAACCCACCAAAAAGAATAATCCTATAGCAAAGAAGGAGCC | |
| | ACACGACAGGGGTAACCTGGGGCTGGAGGGAAGCTGTACTGTCCCACCTAACGTTGCA | |
| | ACTGGGAGACTTCCGGGGGCCTCACTGCTCCTAGAGCCCTCTTCGCTGACTGCCAATA | |
| | TGAAGGAGGTACCTCTGTTCAGGCTACGTCACTTCCCTTGTGGGAATGTCAATTACGG | |
| | CTACCAGCAACAGGGCTTGCCCTTAGAAGCCGCTACTGCCCCTGGAGCTGGTCATTAC | |
| | GAGGATACCATTCTGAAAAGCAAGAATAGCATGAACCAGCCTGGGCCCTGA | |
| ALK- | ATGTCTGGAGAAGTGCGTTTGAGGCAGTTGGAGCAGTTTATTTTGGACGGGCCCGCTC | 2 |
| CDC42BPA | AGACCAATGGGCAGTGCTTCAGTGTGGAGACATTACTGGATATACTCATCTGCCTTTA | |
| | TGATGAATGCAATAATTCTCCATTGAGAAGAGAGAAGAACATTCTCGAATACCTAGAA | |
| | TGGGCTAAACCATTTACTTCTAAAGTGAAACAAATGCGATTACATAGAGAAGACTTTG | |
| | AAATATTAAAGGTGATTGGTCGAGGAGCTTTTGGGGAGGTTGCTGTAGTAAAACTAAA | |
| | AAATGCAGATAAAGTGTTTGCCATGAAAATATTGAATAAATGGGAAATGCTGAAAAGA | |
| | GCTGAGACAGCATGTTTTCGTGAAGAAAGGGATGTATTAGTGAATGGAGACAATAAAT | |
| | GGATTACAACCTTGCACTATGCTTTCCAGGATGACAATAACTTATACCTGGTTATGGA | |
| | TTATTATGTTGGTGGGGATTTGCTTACTCTACTCAGCAAATTTGAAGATAGATTGCCT | |
| | GAAGATATGGCTAGATTTTACTTGGCTGAGATGGTGATAGCAATTGACTCAGTTCATC | |
| | AGCTACATTATGTACACAGAGACATTAAACCTGACAATATACTGATGGATATGAATGG | |
| | ACATATTCGGTTAGCAGATTTTGGTTCTTGTCTGAAGCTGATGGAAGATGGAACGGTT | |
| | CAGTCCTCAGTGGCTGTAGGAACTCCAGATTATATCTCTCCTGAAATCCTTCAAGCCA | |
| | TGGAAGATGGAAAAGGGAGATATGGACCTGAATGTGACTGGTGGTCTTTGGGGGTCTG | |
| | TATGTATGAAATGCTTTACGGAGAAACACCATTTTATGCAGAATCGCTGGTGGAGACA | |
| | TACGGAAAAATCATGAACCACAAAGAGAGGTTTCAGTTTCCAGCCCAAGTGACTGATG | |
| | TGTCTGAAAATGCTAAGGATCTTATTCGAAGGCTCATTTGTAGCAGAGAACATCGACT | |
| | TGGTCAAAATGGAATAGAAGACTTTAAGAAACACCCATTTTTCAGTGGAATTGATTGG | |
| | GATAATATTCGGAACTGTGAAGCACCTTATATTCCAGAAGTTAGTAGCCCAACAGATA | |
| | CATCGAATTTTGATGTAGATGATGATTGTTTAAAAAATTCTGAAACGATGCCCCCACC | |
| | AACACATACTGCATTTTCTGGCCACCATCTGCCATTTGTTGGTTTTACATATACTAGT | |
| | AGCTGTGTACTTTCTGATCGGAGCTGTTTAAGAGTTACGGCTGGTCCCACCTCACTGG | |
| | ATCTTGATGTTAATGTTCAGAGGACTCTAGACAACAACTTAGCAACTGAAGCTTATGA | |
| | AAGAAGAATTAAGCGCCTTGAGCAAGAAAAACTTGAACTCAGTAGAAAACTTCAAGAG | |
| | TCAACACAGACTGTCCAAGCTCTGCAGTATTCAACTGTTGATGGTCCACTAACAGCAA | |
| | GCAAAGATTTAGAAATAAAAAACTTAAAAGAAGAAATTGAAAAACTAAGAAAACAAGT | |
| | AACAGAATCAAGTCATTTGGAACAGCAACTTGAAGAAGCTAATGCTGTGAGGCAAGAA | |
| | CTAGATGATGCTTTTAGACAAATCAAGGCTTATGAAAAACAAATCAAAACGTTACAAC | |
| | AAGAAAGAGAAGATCTAAATAAGGAACTAGTCCAGGCTAGTGAGCGATTAAAAAACCA | |
| | ATCCAAAGAGCTGAAAGACGCACACTGTCAGAGGAAACTGGCCATGCAGGAATTCATG | |
| | GAGATCAATGAGCGGCTAACAGAATTGCACACCCAAAAACAGAAACTTGCTCGCCATG | |
| | TCCGAGATAAGGAAGAAGAGGTGGACCTGGTGATGCAAAAAGTTGAAAGCTTAAGGCA | |
| | AGAACTGCGCAGAACAGAAAGAGCCAAAAAAGAGCTGGAAGTTCATACAGAAGCTCTA | |
| | GCTGCTGAAGCATCTAAAGACAGGAAGCTACGTGAACAGAGTGAGCACTATTCTAAGC | |
| | AACTGGAAAATGAATTGGAGGGACTGAAGCAAAAACAAATTAGTTACTCACCAGGAGT | |
| | ATGCAGCATAGAACATCAGCAAGAGATAACCAAACTAAAGACTGATTTGGAAAAGAAA | |
| | AGTATCTTTTATGAAGAAGAATTATCTAAAAGAGAAGGAATACATGCAAATGAAATAA | |
| | AAAATCTTAAGAAAGAACTGCATGATTCAGAAGGTCAGCAACTTGCTCTCAACAAAGA | |
| | AATTATGATTTTAAAAGACAAATTGGAAAAAACCAGAAGAGAAAGTCAAAGTGAAAGG | |
| | GAGGAATTTGAAAGTGAGTTCAAACAACAATATGAACGAGAAAAAGTGTTGTTAACTG | |
| | AAGAAAATAAAAAGCTGACGAGTGAACTTGATAAGCTTACTACTTTGTATGAGAACTT | |
| | AAGTATACACAACCAGCAGTTAGAAGAAGAGGTTAAAGATCTAGCAGACAAGAAAGAA | |
| | TCAGTTGCACATTGGGAAGCCCAAATCACAGAAATAATTCAGTGGGTCAGCGATGAAA | |
| | AGGATGCACGAGGGTATCTTCAGGCCTTAGCTTCTAAAATGACTGAAGAATTGGAGGC | |
| | ATTAAGAAATTCCAGCTTGGGTACACGAGCAACAGATATGCCCTGGAAAATGCGTCGT | |
| | TTTGCGAAACTGGATATGTCAGCTAGACTGGAGTTGCAGTCGGCTCTGGATGCAGAAA | |
| | TAAGAGCCAAACAGGCCATCCAAGAAGAGTTGAATAAAGTTAAAGCATCTAATATCAT | |
| | AACAGAATGTAAACTAAAAGATTCAGAGAAGAAGAACTTGGAACTACTCTCAGAAATC | |
| | GAACAGCTGATAAAGGACACTGAAGAGCTTAGATCTGAAAAGGTGTACCGCCGGAAGC | |
| | ACCAGGAGCTGCAAGCCATGCAGATGGAGCTGCAGAGCCCTGAGTACAAGCTGAGCAA | |
| | GCTCCGCACCTCGACCATCATGACCGACTACAACCCCAACTACTGCTTTGCTGGCAAG | |
| | ACCTCCTCCATCAGTGACCTGAAGGAGGTGCCGCGGAAAAACATCACCCTCATTCGGG | |
| | GTCTGGGCCATGGCGCCTTTGGGGAGGTGTATGAAGGCCAGGTGTCCGGAATGCCCAA | |
| | CGACCCAAGCCCCCTGCAAGTGGCTGTGAAGACGCTGCCTGAAGTGTGCTCTGAACAG | |
| | GACGAACTGGATTTCCTCATGGAAGCCCTGATCATCAGCAAATTCAACCACCAGAACA | |
| | TTGTTCGCTGCATTGGGGTGAGCCTGCAATCCCTGCCCCGGTTCATCCTGCTGGAGCT | |
| | CATGGCGGGGGGAGACCTCAAGTCCTTCCTCCGAGAGACCCGCCCTCGCCCGAGCCAG | |
| | CCCTCCTCCCTGGCCATGCTGGACCTTCTGCACGTGGCTCGGGACATTGCCTGTGGCT | |
| | GTCAGTATTTGGAGGAAAACCACTTCATCCACCGAGACATTGCTGCCAGAAACTGCCT | |
| | CTTGACCTGTCCAGGCCCTGGAAGAGTGGCCAAGATTGGAGACTTCGGGATGGCCCGA | |
| | GACATCTACAGGGCGAGCTACTATAGAAAGGGAGGCTGTGCCATGCTGCCAGTTAAGT | |
| | GGATGCCCCCAGAGGCCTTCATGGAAGGAATATTCACTTCTAAAACAGACACATGGTC | |
| | CTTTGGAGTGCTGCTATGGGAAATCTTTTCTCTTGGATATATGCCATACCCCAGCAAA | |
| | AGCAACCAGGAAGTTCTGGAGTTTGTCACCAGTGGAGGCCGGATGGACCCACCCAAGA | |
| | ACTGCCCTGGGCCTGTATACCGGATAATGACTCAGTGCTGGCAACATCAGCCTGAAGA | |
| | CAGGCCCAACTTTGCCATCATTTTGGAGAGGATTGAATACTGCACCCAGGACCCGGAT | |
| | GTAATCAACACCGCTTTGCCGATAGAATATGGTCCACTTGTGGAAGAGGAAGAGAAAG | |
| | TGCCTGTGAGGCCCAAGGACCCTGAGGGGGTTCCTCCTCTCCTGGTCTCTCAACAGGC | |
| | AAAACGGGAGGAGGAGCGCAGCCCAGCTGCCCCACCACCTCTGCCTACCACCTCCTCT | |
| | GGCAAGGCTGCAAAGAAACCCACAGCTGCAGAGATCTCTGTTCGAGTCCCTAGAGGGC | |
| | CGGCCGTGGAAGGGGGACACGTGAATATGGCATTCTCTCAGTCCAACCCTCCTTCGGA | |
| | GTTGCACAAGGTCCACGGATCCAGAAACAAGCCCACCAGCTTGTGGAACCCAACGTAC | |
| | GGCTCCTGGTTTACAGAGAAACCCACCAAAAAGAATAATCCTATAGCAAAGAAGGAGC | |
| | CACACGACAGGGGTAACCTGGGGCTGGAGGGAAGCTGTACTGTCCCACCTAACGTTGC | |
| | AACTGGGAGACTTCCGGGGGCCTCACTGCTCCTAGAGCCCTCTTCGCTGACTGCCAAT | |
| | ATGAAGGAGGTACCTCTGTTCAGGCTACGTCACTTCCCTTGTGGGAATGTCAATTACG | |
| | GCTACCAGCAACAGGGCTTGCCCTTAGAAGCCGCTACTGCCCCTGGAGCTGGTCATTA | |
| | CGAGGATACCATTCTGAAAAGCAAGAATAGCATGAACCAGCCTGGGCCCTGA | |
| ALK- | ATGGAGCTCCAGGCAGCCCGCGCCTGCTTCGCCCTGCTGTGGGGCTGTGCGCTGGCCG | 3 |
| EPHA2 | CGGCCGCGGCGGCGCAGGGCAAGGAAGTGGTACTGCTGGACTTTGCTGCAGCTGGAGG | |
| | GGAGCTCGGCTGGCTCACACACCCGTATGGCAAAGGGTGGGACCTGATGCAGAACATC | |
| | ATGAATGACATGCCGATCTACATGTACTCCGTGTGCAACGTGATGTCTGGCGACCAGG | |
| | ACAACTGGCTCCGCACCAACTGGGTGTACCGAGGAGAGGCTGAGCGTATCTTCATTGA | |
| | GCTCAAGTTTACTGTACGTGACTGCAACAGCTTCCCTGGTGGCGCCAGCTCCTGCAAG | |
| | GAGACTTTCAACCTCTACTATGCCGAGTCGGACCTGGACTACGGCACCAACTTCCAGA | |
| | AGCGCCTGTTCACCAAGATTGACACCATTGCGCCCGATGAGATCACCGTCAGCAGCGA | |
| | CTTCGAGGCACGCCACGTGAAGCTGAACGTGGAGGAGCGCTCCGTGGGGCCGCTCACC | |
| | CGCAAAGGCTTCTACCTGGCCTTCCAGGATATCGGTGCCTGTGTGGCGCTGCTCTCCG | |
| | TCCGTGTCTACTACAAGAAGTGCCCCGAGCTGCTGCAGGGCCTGGCCCACTTCCCTGA | |
| | GACCATCGCCGGCTCTGATGCACCTTCCCTGGCCACTGTGGCCGGCACCTGTGTGGAC | |
| | CATGCCGTGGTGCCACCGGGGGGTGAAGAGCCCCGTATGCACTGTGCAGTGGATGGCG | |
| | AGTGGCTGGTGCCCATTGGGCAGTGCCTGTGCCAGGCAGGCTACGAGAAGGTGGAGGA | |
| | TGCCTGCCAGGTGTCACCCACCCCGGAGCCACACCTGCCACTCTCGCTGATCCTCTCT | |
| | GTGGTGACCTCTGCCCTCGTGGCCGCCCTGGTCCTGGCTTTCTCCGGCATCATGATTG | |
| | TGTACCGCCGGAAGCACCAGGAGCTGCAAGCCATGCAGATGGAGCTGCAGAGCCCTGA | |
| | GTACAAGCTGAGCAAGCTCCGCACCTCGACCATCATGACCGACTACAACCCCAACTAC | |
| | TGCTTTGCTGGCAAGACCTCCTCCATCAGTGACCTGAAGGAGGTGCCGCGGAAAAACA | |
| | TCACCCTCATTCGGGGTCTGGGCCATGGCGCCTTTGGGGAGGTGTATGAAGGCCAGGT | |
| | GTCCGGAATGCCCAACGACCCAAGCCCCCTGCAAGTGGCTGTGAAGACGCTGCCTGAA | |
| | GTGTGCTCTGAACAGGACGAACTGGATTTCCTCATGGAAGCCCTGATCATCAGCAAAT | |
| | TCAACCACCAGAACATTGTTCGCTGCATTGGGGTGAGCCTGCAATCCCTGCCCCGGTT | |
| | CATCCTGCTGGAGCTCATGGCGGGGGGAGACCTCAAGTCCTTCCTCCGAGAGACCCGC | |
| | CCTCGCCCGAGCCAGCCCTCCTCCCTGGCCATGCTGGACCTTCTGCACGTGGCTCGGG | |
| | ACATTGCCTGTGGCTGTCAGTATTTGGAGGAAAACCACTTCATCCACCGAGACATTGC | |
| | TGCCAGAAACTGCCTCTTGACCTGTCCAGGCCCTGGAAGAGTGGCCAAGATTGGAGAC | |
| | TTCGGGATGGCCCGAGACATCTACAGGGCGAGCTACTATAGAAAGGGAGGCTGTGCCA | |
| | TGCTGCCAGTTAAGTGGATGCCCCCAGAGGCCTTCATGGAAGGAATATTCACTTCTAA | |
| | AACAGACACATGGTCCTTTGGAGTGCTGCTATGGGAAATCTTTTCTCTTGGATATATG | |
| | CCATACCCCAGCAAAAGCAACCAGGAAGTTCTGGAGTTTGTCACCAGTGGAGGCCGGA | |
| | TGGACCCACCCAAGAACTGCCCTGGGCCTGTATACCGGATAATGACTCAGTGCTGGCA | |
| | ACATCAGCCTGAAGACAGGCCCAACTTTGCCATCATTTTGGAGAGGATTGAATACTGC | |
| | ACCCAGGACCCGGATGTAATCAACACCGCTTTGCCGATAGAATATGGTCCACTTGTGG | |
| | AAGAGGAAGAGAAAGTGCCTGTGAGGCCCAAGGACCCTGAGGGGGTTCCTCCTCTCCT | |
| | GGTCTCTCAACAGGCAAAACGGGAGGAGGAGCGCAGCCCAGCTGCCCCACCACCTCTG | |
| | CCTACCACCTCCTCTGGCAAGGCTGCAAAGAAACCCACAGCTGCAGAGATCTCTGTTC | |
| | GAGTCCCTAGAGGGCCGGCCGTGGAAGGGGGACACGTGAATATGGCATTCTCTCAGTC | |
| | CAACCCTCCTTCGGAGTTGCACAAGGTCCACGGATCCAGAAACAAGCCCACCAGCTTG | |
| | TGGAACCCAACGTACGGCTCCTGGTTTACAGAGAAACCCACCAAAAAGAATAATCCTA | |
| | TAGCAAAGAAGGAGCCACACGACAGGGGTAACCTGGGGCTGGAGGGAAGCTGTACTGT | |
| | CCCACCTAACGTTGCAACTGGGAGACTTCCGGGGGCCTCACTGCTCCTAGAGCCCTCT | |
| | TCGCTGACTGCCAATATGAAGGAGGTACCTCTGTTCAGGCTACGTCACTTCCCTTGTG | |
| | GGAATGTCAATTACGGCTACCAGCAACAGGGCTTGCCCTTAGAAGCCGCTACTGCCCC | |
| | TGGAGCTGGTCATTACGAGGATACCATTCTGAAAAGCAAGAATAGCATGAACCAGCCT | |
| | GGGCCCTGA | |
| ALK- | ATGGCGGTGGCCGAGCTGTACACGCAGTACAACAGGGTCTGGATTCCCGATCCTGAAG | 4 |
| MYO5C | AAGTTTGGAAGTCTGCTGAAATAGCCAAGGACTACAGAGTTGGTGACAAGGTCCTGCG | |
| | ACTCCTGCTGGAGGATGGAACGGAGCTGGATTATTCTGTCAATCCAGAATCTCTGCCT | |
| | CCACTTCGGAATCCTGACATCCTCGTGGGCGAGAATGACCTCACGGCTCTCAGCTATC | |
| | TTCACGAGCCCGCGGTGCTCCACAACCTCAGAATCCGCTTTGCAGAATCCAAACTCAT | |
| | TTACACCTACAGTGGAATCATTTTGGTGGCCATGAATCCTTACAAGCAGTTGCCAATA | |
| | TACGGAGATGCCATCATCCACGCCTACAGCGGGCAGAACATGGGCGATATGGACCCAC | |
| | ACATATTTGCCGTGGCAGAAGAGGCATACAAGCAGATGGCCAGAAACAACAGAAACCA | |
| | GTCCATAATTGTAAGTGGGGAGTCAGGTGCTGGAAAGACAGTGTCGGCTCGCTATGCC | |
| | ATGAGGTACTTTGCCACCGTCAGCAAATCGGGCAGCAACGCTCACGTGGAAGACAAGG | |
| | TCCTGGCATCCAATCCCATCACCGAGGCCGTTGGAAATGCCAAGACCACCCGCAATGA | |
| | CAATAGTAGTCGGTTTGGGAAATACACAGAAATCAGTTTTGATGAACAAAATCAAATT | |
| | ATAGGAGCCAACATGAGCACTTACCTCCTGGAGAAATCCAGAGTTGTCTTTCAATCGG | |
| | AAAATGAACGAAATTACCACATTTTCTATCAGCTTTGTGCATCTGCACAGCAGTCGGA | |
| | ATTTAAACATCTTAAATTGGGGAGTGCCGAAGAATTTAATTATACAAGAATGGGAGGC | |
| | AATACTGTCATTGAGGGTGTGAATGATCGAGCTGAAATGGTAGAGACTCAAAAGACCT | |
| | TCACGCTTCTGGGTTTCAAGGAGGATTTTCAGATGGACGTTTTTAAAATCCTGGCAGC | |
| | CATCCTACATCTGGGCAATGTGCAGATCACCGCGGTGGGCAACGAGAGGTCCTCAGTT | |
| | AGTGAGGATGACAGTCACCTGAAGGTGTTCTGTGAGCTCCTGGGCCTGGAGAGTGGCA | |
| | GAGTTGCTCAGTGGCTGTGCAATCGCAAAATCGTCACAAGCTCTGAGACGGTGGTAAA | |
| | ACCCATGACCAGGCCTCAGGCTGTCAACGCCAGGGATGCACTGGCCAAAAAGATCTAT | |
| | GCTCACCTGTTCGACTTCATTGTGGAGAGAATTAACCAAGCGTTGCAGTTTTCAGGCA | |
| | AGCAGCACACTTTTATTGGTGTTTTGGACATTTATGGTTTTGAAACCTTTGATGTGAA | |
| | CAGCTTTGAACAATTTTGCATCAATTACGCTAATGAAAAACTGCAACAACAGTTTAAC | |
| | ATGCATGTCTTCAAACTGGAACAAGAAGAATACATGAAGGAAGATATACCTTGGACGC | |
| | TGATAGATTTTTATGACAATCAACCAGTTATTGACCTGATTGAAGCAAAAATGGGAAT | |
| | TCTGGAGTTACTGGATGAAGAATGTTTGTTACCACATGGAACTGATGAAAACTGGCTT | |
| | CAAAAGCTGTATAATAATTTTGTCAACAGGAACCCTTTGTTTGAAAAGCCTAGAATGT | |
| | CAAACACATCCTTTGTCATCCAGCACTTTGCTGATAAGGTAGAGTATAAATGTGAAGG | |
| | TTTCCTGGAGAAAAACAGAGACACCGTCTATGACATGCTGGTTGAAATCCTGAGAGCA | |
| | AGCAAGTTTCATCTCTGTGCCAACTTTTTTCAAGAAAATCCAACTCCTCCTTCTCCTT | |
| | TTGGTTCAATGATTACAGTTAAATCTGCAAAGCAAGTCATCAAGCCAAACAGCAAGCA | |
| | TTTCCGGACCACAGTTGGGAGCAAGTTCCGCAGCTCTCTGTACTTGCTCATGGAGACC | |
| | CTCAATGCGACGACGCCCCACTACGTTCGATGCATCAAGCCAAATGATGAGAAGTTAC | |
| | CCTTTGAATTTGACTCCAAAAGAATTGTTCAGCAGCTGCGAGCCTGCGGCGTTTTAGA | |
| | AACGATTCGCATTAGTGCACAGAGCTACCCTTCCAGGTGGACATACATCGAGTTCTAC | |
| | AGTCGCTACGGCATTCTCATGACCAAGCAAGAGCTTTCCTTCAGCGATAAAAAGGAGG | |
| | TGTGCAAGGTGGTTTTACACAGACTCATCCAGGATTCTAATCAGTACCAGTTTGGTAA | |
| | AACCAAAATTTTCTTCAGAGCAGGACAAGTGGCTTATTTAGAGAAACTTCGATTGGAT | |
| | AAACTGAGGCAGAGTTGTGTTATGGTACAAAAGCACATGCGTGGCTGGCTCCAGAGGA | |
| | AAAAATTCCTCCGAGAGAGACGAGCCGCCCTGATAATCCAGCAGTACTTCCGGGGTCA | |
| | GCAAACTGTGAGGAAAGCTATTACTGCAGTGGCCTTAAAAGAAGCTTGGGCAGCCATA | |
| | ATCATTCAGAAGCACTGCCGCGGGTATCTTGTTCGCAGCCTGTATCAGTTGATTCGCA | |
| | TGGCCACCATCACAATGCAGGCCTACAGCCGAGGATTCCTGGCAAGGAGGAGGTATCG | |
| | AAAGATGCTGGAGGAACATAAGGCTGTGATCCTACAGAAATACGCACGGGCGTGGCTG | |
| | GCCAGACGCAGATTCCAGAGTATCCGACGATTCGTGCTTAATATTCAGCTTACTTACA | |
| | GGGTCCAGCGTTTGCAGAAAAAGTTGGAAGATCAGAACAAAGAAAACCATGGGCTGGT | |
| | GGAGAAGCTGACTAGCCTGGCTGCTCTTCGAGCTGGGGATGTGGAAAAGATTCAGAAG | |
| | CTGGAAGCAGAACTAGAAAAAGCAGCCACTCACAGGCGAAATTACGAGGAGAAGGGGA | |
| | AGAGATACAGGGATGCTGTGGAAGAGAAATTGGCAAAGCTTCAGAAGCATAATTCAGA | |
| | ACTGGAAACACAGAAAGAACAAATACAGCTGAAGCTTCAAGAGAAGACTGAAGAGTTA | |
| | AAAGAAAAAATGGACAACCTCACCAAGCAGCTCTTTGATGATGTACAAAAGGAAGAAC | |
| | GGCAAAGAATGCTTCTTGAAAAAAGTTTTGAACTGAAAACACAAGACTATGAGAAGCA | |
| | GATTCAGTCTTTGAAAGAAGAAATTAAAGCTCTCAAGGATGAGAAGATGCAACTCCAA | |
| | CACCTGGTGGAGGGGGAGCACGTCACTTCTGATGGCTTGAAGGCGGAAGTGGCCCGCC | |
| | TGAGCAAGCAGGTCAAGACAATCTCTGAGTTCGAAAAAGAGATAGAACTACTTCAGGC | |
| | ACAGAAGATAGATGTGGAGAAACATGTGCAGTCACAAAAACGGGAAATGAGAGAAAAG | |
| | ATGTCAGAGATCACCAAACAACTTCTCGAAAGCTATGACATTGAAGATGTAAGAAGCA | |
| | GGCTCTCTGTGGAAGATCTGGAACATTTAAATGAGGATGGAGAACTTTGGTTTGCTTA | |
| | TGAAGGACTAAAGAAAGCAACACGTGTTTTGGAGAGCCATTTCCAGTCTCAGAAGGAT | |
| | TGCTATGAAAAGGAGATTGAAGCTTTGAACTTCAAAGTGGTGCATCTCAGTCAAGAAA | |
| | TCAACCACCTGCAGAAGTTATTCAGAGAAGAAAATGACATCAATGAAAGCATCCGTCA | |
| | TGAAGTTACCAGGCTAACATCAGAGAACATGATGATCCCAGACTTTAAACAGCAAATT | |
| | TCAGAATTGGAGAAACAGAAGCAAGATCTTGAAATCCGCCTGAATGAACAAGCTGAGA | |
| | AAATGAAAGTGTACCGCCGGAAGCACCAGGAGCTGCAAGCCATGCAGATGGAGCTGCA | |
| | GAGCCCTGAGTACAAGCTGAGCAAGCTCCGCACCTCGACCATCATGACCGACTACAAC | |
| | CCCAACTACTGCTTTGCTGGCAAGACCTCCTCCATCAGTGACCTGAAGGAGGTGCCGC | |
| | GGAAAAACATCACCCTCATTCGGGGTCTGGGCCATGGCGCCTTTGGGGAGGTGTATGA | |
| | AGGCCAGGTGTCCGGAATGCCCAACGACCCAAGCCCCCTGCAAGTGGCTGTGAAGACG | |
| | CTGCCTGAAGTGTGCTCTGAACAGGACGAACTGGATTTCCTCATGGAAGCCCTGATCA | |
| | TCAGCAAATTCAACCACCAGAACATTGTTCGCTGCATTGGGGTGAGCCTGCAATCCCT | |
| | GCCCCGGTTCATCCTGCTGGAGCTCATGGCGGGGGGAGACCTCAAGTCCTTCCTCCGA | |
| | GAGACCCGCCCTCGCCCGAGCCAGCCCTCCTCCCTGGCCATGCTGGACCTTCTGCACG | |
| | TGGCTCGGGACATTGCCTGTGGCTGTCAGTATTTGGAGGAAAACCACTTCATCCACCG | |
| | AGACATTGCTGCCAGAAACTGCCTCTTGACCTGTCCAGGCCCTGGAAGAGTGGCCAAG | |
| | ATTGGAGACTTCGGGATGGCCCGAGACATCTACAGGGCGAGCTACTATAGAAAGGGAG | |
| | GCTGTGCCATGCTGCCAGTTAAGTGGATGCCCCCAGAGGCCTTCATGGAAGGAATATT | |
| | CACTTCTAAAACAGACACATGGTCCTTTGGAGTGCTGCTATGGGAAATCTTTTCTCTT | |
| | GGATATATGCCATACCCCAGCAAAAGCAACCAGGAAGTTCTGGAGTTTGTCACCAGTG | |
| | GAGGCCGGATGGACCCACCCAAGAACTGCCCTGGGCCTGTATACCGGATAATGACTCA | |
| | GTGCTGGCAACATCAGCCTGAAGACAGGCCCAACTTTGCCATCATTTTGGAGAGGATT | |
| | GAATACTGCACCCAGGACCCGGATGTAATCAACACCGCTTTGCCGATAGAATATGGTC | |
| | CACTTGTGGAAGAGGAAGAGAAAGTGCCTGTGAGGCCCAAGGACCCTGAGGGGGTTCC | |
| | TCCTCTCCTGGTCTCTCAACAGGCAAAACGGGAGGAGGAGCGCAGCCCAGCTGCCCCA | |
| | CCACCTCTGCCTACCACCTCCTCTGGCAAGGCTGCAAAGAAACCCACAGCTGCAGAGA | |
| | TCTCTGTTCGAGTCCCTAGAGGGCCGGCCGTGGAAGGGGGACACGTGAATATGGCATT | |
| | CTCTCAGTCCAACCCTCCTTCGGAGTTGCACAAGGTCCACGGATCCAGAAACAAGCCC | |
| | ACCAGCTTGTGGAACCCAACGTACGGCTCCTGGTTTACAGAGAAACCCACCAAAAAGA | |
| | ATAATCCTATAGCAAAGAAGGAGCCACACGACAGGGGTAACCTGGGGCTGGAGGGAAG | |
| | CTGTACTGTCCCACCTAACGTTGCAACTGGGAGACTTCCGGGGGCCTCACTGCTCCTA | |
| | GAGCCCTCTTCGCTGACTGCCAATATGAAGGAGGTACCTCTGTTCAGGCTACGTCACT | |
| | TCCCTTGTGGGAATGTCAATTACGGCTACCAGCAACAGGGCTTGCCCTTAGAAGCCGC | |
| | TACTGCCCCTGGAGCTGGTCATTACGAGGATACCATTCTGAAAAGCAAGAATAGCATG | |
| | AACCAGCCTGGGCCCTGA | |
| ALK- | ATGGAGGTGGCGGTGGAGAAGGCGGTGGCGGCGGCGGCAGCGGCCTCGGCTGCGGCCT | 5 |
| TRIM24 | CCGGGGGGCCCTCGGCGGCGCCGAGCGGGGAGAACGAGGCCGAGAGTCGGCAGGGCCC | |
| | GGACTCGGAGCGCGGCGGCGAGGCGGCCCGGCTCAACCTGTTGGACACTTGCGCCGTG | |
| | TGCCACCAGAACATCCAGAGCCGGGCGCCCAAGCTGCTGCCCTGCCTGCACTCTTTCT | |
| | GCCAGCGCTGCCTGCCCGCGCCCCAGCGCTACCTCATGCTGCCCGCGCCCATGCTGGG | |
| | CTCGGCCGAGACCCCGCCACCCGTCCCTGCCCCCGGCTCGCCGGTCAGCGGCTCGTCG | |
| | CCGTTCGCCACCCAAGTTGGAGTCATTCGTTGCCCAGTTTGCAGCCAAGAATGTGCAG | |
| | AGAGACACATCATAGATAACTTTTTTGTGAAGGACACTACTGAGGTTCCCAGCAGTAC | |
| | AGTAGAAAAGTCAAATCAGGTATGTACAAGCTGTGAGGACAACGCAGAAGCCAATGGG | |
| | TTTTGTGTAGAGTGTGTTGAATGGCTCTGCAAGACGTGTATCAGAGCTCATCAGAGGG | |
| | TAAAGTTCACAAAAGACCACACTGTCAGACAGAAAGAGGAAGTATCTCCAGAGGCAGT | |
| | TGGTGTCACCAGCCAGCGACCAGTGTTTTGTCCTTTTCATAAAAAGGAGCAGCTGAAG | |
| | CTGTACTGTGAGACATGTGACAAACTGACATGTCGAGACTGTCAGTIGTTAGAACATA | |
| | AAGAGCATAGATACCAATTTATAGAAGAAGCTTTTCAGAATCAGAAAGTGATCATAGA | |
| | TACACTAATCACCAAACTGATGGAAAAAACAAAATACATAAAATTCACAGGAAATCAG | |
| | ATCCAAAACAGAATTATTGAAGTAAATCAAAATCAAAAGCAGGTGGAACAGGATATTA | |
| | AAGTTGCTATATTTACACTGATGGTAGAAATAAATAAAAAAGGAAAAGCTCTACTGCA | |
| | TCAGTTAGAGAGCCTTGCAAAGGACCATCGCATGAAACTTATGCAACAACAACAGGAA | |
| | GTGGCTGGACTCTCTAAACAATTGGAGCATGTCATGCATTTTTCTAAATGGGCAGTTT | |
| | CCAGTGGCAGCAGTACAGCATTACTTTATAGCAAACGACTGATTACATACCGGTTACG | |
| | GCACCTCCTTCGTGCAAGGTGTGATGCATCCCCAGTGACCAACAACACCATCCAATTT | |
| | CACTGTGATCCTAGTTTCTGGGCTCAAAATATCATCAACTTAGGTTCTTTAGTAATCG | |
| | AGGATAAAGAGAGCCAGCCACAAATGCCTAAGCAGAATCCTGTCGTGGAACAGAATTC | |
| | ACAGCCACCAAGTGGTTTATCATCAAACCAGTTATCCAAGTTCCCAACACAGATCAGC | |
| | CTAGCTCAATTACGGCTCCAGCATATGCAGCAACAGCAACCGCCTCCACGTTTGATAA | |
| | ACTTTCAGAATCACAGCCCCAAACCCAATGGACCAGTTCTTCCTCCTCATCCTCAACA | |
| | ACTGAGATATCCACCAAACCAGAACATACCACGACAAGCAATAAAGCCAAACCCCCTA | |
| | CAGATGGCTTTCTTGGCTCAACAAGCCATAAAACAGTGGCAGATCAGCAGTGGACAGG | |
| | GAACCCCATCAACTACCAACAGCACATCCTCTACTCCTTCCAGCCCCACGATTACTAG | |
| | TGCAGCAGGATATGATGGAAAGGCTTTTGGTTCACCTATGATCGATTTGAGCTCACCA | |
| | GTGGGAGGGTCTTATAATCTTCCCTCTCTTCCGGATATTGACTGTTCAAGTACTATTA | |
| | TGCTGGACAATATTGTGAGGAAAGATACTAATATAGATCATGGCCAGCCAAGACCACC | |
| | CTCAAACAGAACGGTCCAGTCACCAAATTCATCAGTGCCATCTCCAGGCCTTGCAGTG | |
| | TACCGCCGGAAGCACCAGGAGCTGCAAGCCATGCAGATGGAGCTGCAGAGCCCTGAGT | |
| | ACAAGCTGAGCAAGCTCCGCACCTCGACCATCATGACCGACTACAACCCCAACTACTG | |
| | CTTTGCTGGCAAGACCTCCTCCATCAGTGACCTGAAGGAGGTGCCGCGGAAAAACATC | |
| | ACCCTCATTCGGGGTCTGGGCCATGGCGCCTTTGGGGAGGTGTATGAAGGCCAGGTGT | |
| | CCGGAATGCCCAACGACCCAAGCCCCCTGCAAGTGGCTGTGAAGACGCTGCCTGAAGT | |
| | GTGCTCTGAACAGGACGAACTGGATTTCCTCATGGAAGCCCTGATCATCAGCAAATTC | |
| | AACCACCAGAACATTGTTCGCTGCATTGGGGTGAGCCTGCAATCCCTGCCCCGGTTCA | |
| | TCCTGCTGGAGCTCATGGCGGGGGGAGACCTCAAGTCCTTCCTCCGAGAGACCCGCCC | |
| | TCGCCCGAGCCAGCCCTCCTCCCTGGCCATGCTGGACCTTCTGCACGTGGCTCGGGAC | |
| | ATTGCCTGTGGCTGTCAGTATTTGGAGGAAAACCACTTCATCCACCGAGACATTGCTG | |
| | CCAGAAACTGCCTCTTGACCTGTCCAGGCCCTGGAAGAGTGGCCAAGATTGGAGACTT | |
| | CGGGATGGCCCGAGACATCTACAGGGCGAGCTACTATAGAAAGGGAGGCTGTGCCATG | |
| | CTGCCAGTTAAGTGGATGCCCCCAGAGGCCTTCATGGAAGGAATATTCACTTCTAAAA | |
| | CAGACACATGGTCCTTTGGAGTGCTGCTATGGGAAATCTTTTCTCTTGGATATATGCC | |
| | ATACCCCAGCAAAAGCAACCAGGAAGTTCTGGAGTTTGTCACCAGTGGAGGCCGGATG | |
| | GACCCACCCAAGAACTGCCCTGGGCCTGTATACCGGATAATGACTCAGTGCTGGCAAC | |
| | ATCAGCCTGAAGACAGGCCCAACTTTGCCATCATTTTGGAGAGGATTGAATACTGCAC | |
| | CCAGGACCCGGATGTAATCAACACCGCTTTGCCGATAGAATATGGTCCACTTGTGGAA | |
| | GAGGAAGAGAAAGTGCCTGTGAGGCCCAAGGACCCTGAGGGGGTTCCTCCTCTCCTGG | |
| | TCTCTCAACAGGCAAAACGGGAGGAGGAGCGCAGCCCAGCTGCCCCACCACCTCTGCC | |
| | TACCACCTCCTCTGGCAAGGCTGCAAAGAAACCCACAGCTGCAGAGATCTCTGTTCGA | |
| | GTCCCTAGAGGGCCGGCCGTGGAAGGGGGACACGTGAATATGGCATTCTCTCAGTCCA | |
| | ACCCTCCTTCGGAGTTGCACAAGGTCCACGGATCCAGAAACAAGCCCACCAGCTTGTG | |
| | GAACCCAACGTACGGCTCCTGGTTTACAGAGAAACCCACCAAAAAGAATAATCCTATA | |
| | GCAAAGAAGGAGCCACACGACAGGGGTAACCTGGGGCTGGAGGGAAGCTGTACTGTCC | |
| | CACCTAACGTTGCAACTGGGAGACTTCCGGGGGCCTCACTGCTCCTAGAGCCCTCTTC | |
| | GCTGACTGCCAATATGAAGGAGGTACCTCTGTTCAGGCTACGTCACTTCCCTTGTGGG | |
| | AATGTCAATTACGGCTACCAGCAACAGGGCTTGCCCTTAGAAGCCGCTACTGCCCCTG | |
| | GAGCTGGTCATTACGAGGATACCATTCTGAAAAGCAAGAATAGCATGAACCAGCCTGG | |
| | GCCCTGA | |
| ALK- | ATGCAGGCCGCCGCCCTCCCTGAGGAGATCCGTTGGCTCCTGGAAGATGCTGAAGAGT | 6 |
| SKAP1 | TTCTGGCAGAAGGTTTGCGGAATGAGAACCTCAGCGCTGTTGCAAGGGATCACAGAGA | |
| | CCATATTCTACGGGGCTTTCAGCAAATCAAAGCCAGGTACTATTGGGATTTTCAGCCC | |
| | CAAGTGTACCGCCGGAAGCACCAGGAGCTGCAAGCCATGCAGATGGAGCTGCAGAGCC | |
| | CTGAGTACAAGCTGAGCAAGCTCCGCACCTCGACCATCATGACCGACTACAACCCCAA | |
| | CTACTGCTTTGCTGGCAAGACCTCCTCCATCAGTGACCTGAAGGAGGTGCCGCGGAAA | |
| | AACATCACCCTCATTCGGGGTCTGGGCCATGGCGCCTTTGGGGAGGTGTATGAAGGCC | |
| | AGGTGTCCGGAATGCCCAACGACCCAAGCCCCCTGCAAGTGGCTGTGAAGACGCTGCC | |
| | TGAAGTGTGCTCTGAACAGGACGAACTGGATTTCCTCATGGAAGCCCTGATCATCAGC | |
| | AAATTCAACCACCAGAACATTGTTCGCTGCATTGGGGTGAGCCTGCAATCCCTGCCCC | |
| | GGTTCATCCTGCTGGAGCTCATGGCGGGGGGAGACCTCAAGTCCTTCCTCCGAGAGAC | |
| | CCGCCCTCGCCCGAGCCAGCCCTCCTCCCTGGCCATGCTGGACCTTCTGCACGTGGCT | |
| | CGGGACATTGCCTGTGGCTGTCAGTATTTGGAGGAAAACCACTTCATCCACCGAGACA | |
| | TTGCTGCCAGAAACTGCCTCTTGACCTGTCCAGGCCCTGGAAGAGTGGCCAAGATTGG | |
| | AGACTTCGGGATGGCCCGAGACATCTACAGGGCGAGCTACTATAGAAAGGGAGGCTGT | |
| | GCCATGCTGCCAGTTAAGTGGATGCCCCCAGAGGCCTTCATGGAAGGAATATTCACTT | |
| | CTAAAACAGACACATGGTCCTTTGGAGTGCTGCTATGGGAAATCTTTTCTCTTGGATA | |
| | TATGCCATACCCCAGCAAAAGCAACCAGGAAGTTCTGGAGTTTGTCACCAGTGGAGGC | |
| | CGGATGGACCCACCCAAGAACTGCCCTGGGCCTGTATACCGGATAATGACTCAGTGCT | |
| | GGCAACATCAGCCTGAAGACAGGCCCAACTTTGCCATCATTTTGGAGAGGATTGAATA | |
| | CTGCACCCAGGACCCGGATGTAATCAACACCGCTTTGCCGATAGAATATGGTCCACTT | |
| | GTGGAAGAGGAAGAGAAAGTGCCTGTGAGGCCCAAGGACCCTGAGGGGGTTCCTCCTC | |
| | TCCTGGTCTCTCAACAGGCAAAACGGGAGGAGGAGCGCAGCCCAGCTGCCCCACCACC | |
| | TCTGCCTACCACCTCCTCTGGCAAGGCTGCAAAGAAACCCACAGCTGCAGAGATCTCT | |
| | GTTCGAGTCCCTAGAGGGCCGGCCGTGGAAGGGGGACACGTGAATATGGCATTCTCTC | |
| | AGTCCAACCCTCCTTCGGAGTTGCACAAGGTCCACGGATCCAGAAACAAGCCCACCAG | |
| | CTTGTGGAACCCAACGTACGGCTCCTGGTTTACAGAGAAACCCACCAAAAAGAATAAT | |
| | CCTATAGCAAAGAAGGAGCCACACGACAGGGGTAACCTGGGGCTGGAGGGAAGCTGTA | |
| | CTGTCCCACCTAACGTTGCAACTGGGAGACTTCCGGGGGCCTCACTGCTCCTAGAGCC | |
| | CTCTTCGCTGACTGCCAATATGAAGGAGGTACCTCTGTTCAGGCTACGTCACTTCCCT | |
| | TGTGGGAATGTCAATTACGGCTACCAGCAACAGGGCTTGCCCTTAGAAGCCGCTACTG | |
| | CCCCTGGAGCTGGTCATTACGAGGATACCATTCTGAAAAGCAAGAATAGCATGAACCA | |
| | GCCTGGGCCCTGA | |
| ALK- | ATGTTCACCCTGTCTCAGACCTCGAGAGCATGGTTCATCGATAGAGCCCGTCAGGCAC | 7 |
| UBE3B | GAGAAGAAAGGCTTGTGCAGAAGGAACGGGAGCGGGCAGCTGTTGTGATCCAGGCCCA | |
| | TGTCCGGAGTTTTCTCTGTCGGAGTCGACTGCAGAGAGATATCAGGAGAGAGATTGAT | |
| | GACTTTTTTAAAGCAGATGACCCTGAGTCCACTAAAAGAAGTGCACTTTGTATTTTCA | |
| | AGATTGCCAGGAAACTGCTGTTCCTATTCAGAATCAAAGAGGATAATGAGAGATTTGA | |
| | GAAGTTGTGTCGCAGCATCCTGAGCAGCATGGATGCTGAGAATGAGCCTAAGGTGTGG | |
| | TATGTGTCCCTGGCTTGTTCTAAGGACCTCACCCTCCTTTGGATTCAACAGATCAAGA | |
| | ACATTTTGTGGTACTGCTGTGATTTTCTCAAGCAGCTCAAGCCTGAAATCCTGCAGGA | |
| | CTCCCGACTCATCACCCTGTACCTCACGATGCTTGTCACCTTCACAGACACTTCAACG | |
| | TGGAAAATTCTTCGGGGAAAAGGTGAAAGTCTTCGACCAGCGATGAACCACATTTGTG | |
| | CAAATATAATGGGACATCTCAACCAGCATGGATTTTATTCTGTGCTGCAGATATTGTT | |
| | AACCCGTGGCCTGGCAAGACCCCGTCCTTGTCTATCCAAAGGCACTTTAACAGCAGCT | |
| | TTTTCTCTAGCGTTACGCCCTGTGATTGCTGCACAGTTCTCAGACAATCTGATTCGGC | |
| | CGTTCCTCATCCACATCATGTCTGTGCCTGCTCTGGTGACTCATCTCAGCACAGTGAC | |
| | CCCTGAGCGCCTCACTGTTTTAGAATCCCATGACATGCTTCGTAAATTCATCATATTT | |
| | TTAAGAGACCAAGATCGATGCCGTGATGTATGTGAAAGTTTAGAAGGATGCCATACGC | |
| | TTTGTCTAATGGTGTACCGCCGGAAGCACCAGGAGCTGCAAGCCATGCAGATGGAGCT | |
| | GCAGAGCCCTGAGTACAAGCTGAGCAAGCTCCGCACCTCGACCATCATGACCGACTAC | |
| | AACCCCAACTACTGCTTTGCTGGCAAGACCTCCTCCATCAGTGACCTGAAGGAGGTGC | |
| | CGCGGAAAAACATCACCCTCATTCGGGGTCTGGGCCATGGCGCCTTTGGGGAGGTGTA | |
| | TGAAGGCCAGGTGTCCGGAATGCCCAACGACCCAAGCCCCCTGCAAGTGGCTGTGAAG | |
| | ACGCTGCCTGAAGTGTGCTCTGAACAGGACGAACTGGATTTCCTCATGGAAGCCCTGA | |
| | TCATCAGCAAATTCAACCACCAGAACATTGTTCGCTGCATTGGGGTGAGCCTGCAATC | |
| | CCTGCCCCGGTTCATCCTGCTGGAGCTCATGGCGGGGGGAGACCTCAAGTCCTTCCTC | |
| | CGAGAGACCCGCCCTCGCCCGAGCCAGCCCTCCTCCCTGGCCATGCTGGACCTTCTGC | |
| | ACGTGGCTCGGGACATTGCCTGTGGCTGTCAGTATTTGGAGGAAAACCACTTCATCCA | |
| | CCGAGACATTGCTGCCAGAAACTGCCTCTTGACCTGTCCAGGCCCTGGAAGAGTGGCC | |
| | AAGATTGGAGACTTCGGGATGGCCCGAGACATCTACAGGGCGAGCTACTATAGAAAGG | |
| | GAGGCTGTGCCATGCTGCCAGTTAAGTGGATGCCCCCAGAGGCCTTCATGGAAGGAAT | |
| | ATTCACTTCTAAAACAGACACATGGTCCTTTGGAGTGCTGCTATGGGAAATCTTTTCT | |
| | CTTGGATATATGCCATACCCCAGCAAAAGCAACCAGGAAGTTCTGGAGTTTGTCACCA | |
| | GTGGAGGCCGGATGGACCCACCCAAGAACTGCCCTGGGCCTGTATACCGGATAATGAC | |
| | TCAGTGCTGGCAACATCAGCCTGAAGACAGGCCCAACTTTGCCATCATTTTGGAGAGG | |
| | ATTGAATACTGCACCCAGGACCCGGATGTAATCAACACCGCTTTGCCGATAGAATATG | |
| | GTCCACTTGTGGAAGAGGAAGAGAAAGTGCCTGTGAGGCCCAAGGACCCTGAGGGGGT | |
| | TCCTCCTCTCCTGGTCTCTCAACAGGCAAAACGGGAGGAGGAGCGCAGCCCAGCTGCC | |
| | CCACCACCTCTGCCTACCACCTCCTCTGGCAAGGCTGCAAAGAAACCCACAGCTGCAG | |
| | AGATCTCTGTTCGAGTCCCTAGAGGGCCGGCCGTGGAAGGGGGACACGTGAATATGGC | |
| | ATTCTCTCAGTCCAACCCTCCTTCGGAGTTGCACAAGGTCCACGGATCCAGAAACAAG | |
| | CCCACCAGCTTGTGGAACCCAACGTACGGCTCCTGGTTTACAGAGAAACCCACCAAAA | |
| | AGAATAATCCTATAGCAAAGAAGGAGCCACACGACAGGGGTAACCTGGGGCTGGAGGG | |
| | AAGCTGTACTGTCCCACCTAACGTTGCAACTGGGAGACTTCCGGGGGCCTCACTGCTC | |
| | CTAGAGCCCTCTTCGCTGACTGCCAATATGAAGGAGGTACCTCTGTTCAGGCTACGTC | |
| | ACTTCCCTTGTGGGAATGTCAATTACGGCTACCAGCAACAGGGCTTGCCCTTAGAAGC | |
| | CGCTACTGCCCCTGGAGCTGGTCATTACGAGGATACCATTCTGAAAAGCAAGAATAGC | |
| | ATGAACCAGCCTGGGCCCTGA | |
| ALK-TNS3 | ATGGAGGAGGGCCATGGGCTGGACCTCACTTACATCACGGAGCGCATCATCGCTGTGT | 8 |
| | CCTTCCCTGCCGGCTGCTCTGAGGAGTCCTACCTGCACAACCTACAGGAGGTCACGCG | |
| | CATGCTCAAGTCCAAGCACGGGGACAACTACCTGGTATTAAACCTTTCAGAAAAGAGA | |
| | TATGACCTTACGAAGCTTAACCCAAAGATCATGGATGTGGGCTGGCCAGAGCTCCACG | |
| | CACCGCCCCTGGATAAGATGTGTACCATATGCAAGGCGCAGGAGTCCTGGCTGAACAG | |
| | CAACCTCCAGCATGTGGTCGTCATTCACTGCAGGGGCGGGAAAGGACGCATAGGAGTG | |
| | GTCATATCATCCTACATGCATTTCACCAACGTCTCAGCCAGCGCCGACCAGGCCCTTG | |
| | ACAGGTTTGCAATGAAGAAGTTTTATGATGACAAAGTTTCAGCTTTAATGCAGCCTTC | |
| | CCAAAAACGGTATGTTCAGTTCCTCAGTGGGCTCCTGTCCGGATCGGTGAAAATGAAT | |
| | GCCTCTCCCCTGTTCCTGCATTTTGTCATCCTCCACGGCACCCCCAACTTCGACACAG | |
| | GTGGAGTGTGCCGGCCCTTTCTGAAGCTCTACCAAGCCATGCAGCCTGTGTACACCTC | |
| | CGGGATCTACAACGTTGGCCCAGAAAACCCCAGCAGGATCTGCATCGTCATCGAGCCG | |
| | GCCCAGCTTCTGAAGGGAGATGTCATGGTGAAATGCTACCACAAGAAATACCGCTCGG | |
| | CCACCCGTGACGTCATTTTCCGCCTGCAGTTTCACACTGGGGCTGTGCAGGGCTACGG | |
| | GCTGGTGTTTGGGAAGGAGGATCTGGACAATGCCAGCAAAGATGACCGTTTTCCTGAC | |
| | TATGGGAAGGTTGAATTAGTCTTCTCTGCCACGCCTGAGAAGATTCAAGGGTCCGAAC | |
| | ACTTGTACAACGACCACGGTGTGATTGTGGACTACAACACAACAGACCCACTGATACG | |
| | CTGGGACTCGTACGAGAACCTCAGTGCAGATGGAGAAGTGCTACACACGCAGGGCCCT | |
| | GTCGATGGCAGCCTTTACGCGAAGGTGAGGAAGAAAAGCTCCTCGGATCCTGGCATCC | |
| | CAGGTGGCCCCCAGGCAATCCCGGCCACCAACAGCCCAGACCACAGTGACCACACCTT | |
| | GTCTGTCAGCAGTGACTCCGGCCACTCTACAGCCTCTGCCAGGACGGATAAGACGGAA | |
| | GAGCGCCTGGCCCCAGGAACCAGGAGGGGCCTGAGTGCCCAGGAGAAGGCAGAGTTGG | |
| | ACCAGCTGCTCAGTGGCTTTGGCCTGGAAGATCCTGGAAGCTCCCTCAAGGAAATGAC | |
| | TGATGCTCGAAGCAAGTACAGTGGGACCCGCCACGTGGTGCCAGCCCAGGTTCACGTG | |
| | AATGGAGACGCTGCTCTGAAGGATCGGGAGACAGACATTCTGGATGACGAGATGCCCC | |
| | ACCACGACCTGCACAGTGTGGACAGCCTTGGGACCCTGTCCTCCTCGGAAGGGCCTCA | |
| | GTCGGCCCACCTGGGTCCCTTCACCTGCCACAAGAGCAGCCAGAACTCACTCCTATCT | |
| | GACGGTTTTGGCAGCAACGTTGGTGAAGATCCGCAGGGCACCCTCGTTCCGGACCTGG | |
| | GCCTTGGCATGGACGGCCCCTATGAGCGGGAGCGGACTTTTGGGAGTCGAGAGCCCAA | |
| | GCAGCCCCAGCCCCTGCTGAGAAAGCCCTCAGTGTCCGCCCAGATGCAGGCCTATGGG | |
| | CAGAGCAGCTACTCCACACAGACCTGGGTGCGCCAGCAGCAGATGGTTGTAGCTCACC | |
| | AGTATAGCTTCGCCCCAGATGGGGAGGCCCGGCTGGTGAGCCGCTGCCCTGCAGACAA | |
| | TCCTGGCCTCGTCCAGGCCCAGCCCAGAGTGCCACTCACCCCCACCCGAGGGACCAGC | |
| | AGTAGGGTGGCTGTCCAGAGGGGTGTAGGCAGTGGGCCACATCCCCCTGACACACAGC | |
| | AGCCCTCTCCCAGCAAAGCGTTCAAACCCAGGTTTCCAGGAGACCAGGTTGTGAATGG | |
| | AGCCGGCCCAGAGCTGAGCACAGGCCCCTCCCCAGGCTCGCCCACCCTGGACATCGAC | |
| | CAGTCCATCGAGCAGCTCAACAGGCTGATCCTGGAGCTGGATCCCACCTTCGAGCCCA | |
| | TCCCTACCCACATGAACGCCCTCGGTAGCCAGGCCAATGGCTCTGTGTCTCCAGACAG | |
| | CGTGGGAGGTGGGCTCCGGGCAAGCAGCAGGCTGCCTGACACAGGAGAGGGCCCCAGC | |
| | AGGGCCACCGGGCGGCAAGGCTCCTCTGCTGAACAGCCCCTGGGCGGGAGACTCAGGA | |
| | AGCTGAGCCTGGGGCAGTACGACAACGATGCTGGGGGGCAGCTGCCCTTCTCCAAATG | |
| | TGCATGGGGAAAGGCTGGTGTGGACTATGCCCCAAACCTGCCGCCATTCCCCTCACCA | |
| | GCGGACGTCAAAGAGACGATGACCCCTGGCTATCCCCAGGACCTCGATATTATCGATG | |
| | GCAGAATTTTAAGTAGCAAGGAGTCCATGTGTTCAACTCCAGCATTTCCTGTGTCTCC | |
| | AGAGACACCGTATGTGAAAACAGCGCTGCGCCATCCTCCGTTCAGCCCACCTGAGCCC | |
| | CCGCTGAGCAGCCCAGCCAGTCAGCACAAAGGAGGACGTGAACCACGAAGCTGCCCTG | |
| | AGACGCTCACTCACGCTGTGGGGATGTCAGAGAGCCCCATCGGACCCAAATCCACGAT | |
| | GCTCCGGGCTGATGCGTCCTCGACGCCCTCCTTTCAGCAGGCTTTTGCTTCTTCCTGC | |
| | ACCATTTCCAGCAACGGCCCTGGGCAGAGGAGAGAGAGCTCCTCTTCTGCAGAACGCC | |
| | AGTGGGTGGAGAGCAGCCCCAAGCCCATGGTTTCCCTGCTGGGGAGCGGCCGGCCCAC | |
| | CGGAAGTCCCCTCAGCGCTGAGTTCTCCGGTACCAGGAAGGACTCCCCAGTGCTGTCC | |
| | TGCTTCCCGCCGTCAGAGCTCCAGGCTCCTTTCCACAGCCATGAGCTGTCCCTAGCAG | |
| | AGCCACCGGACTCCCTGGCGCCTCCCAGCAGCCAGGCCTTCCTGGGCTTCGGCACCGC | |
| | CCCAGTGGGAAGTGGCCTTCCGCCCGAGGAGGACCTGGGGGCCTTGCTGGCCAATTCT | |
| | CATGGAGCGTCACCGACCCCCAGCATCCCGCTGACAGCGACAGGGGCTGCCGACAATG | |
| | GCTTCCTGTCCCACAACTTTCTCACGGTGGCGCCTGGACACAGCAGCCACCACAGTCC | |
| | AGGCCTGCAGGGCCAGGGTGTGACCCTGCCCGGGCAGCCACCCCTCCCTGAGAAGAAG | |
| | CGGGCCTCGGAGGGGGATCGTTCTTTGGGCTCAGTCTCTCCCTCCTCCAGTGGCTTCT | |
| | CCAGCCCGCACAGCGGGAGCACCATCAGTATCCCCTTCCCAAATGTCCTTCCCGACTT | |
| | TTCCAAGGCTTCAGAAGCGGCCTCACCTCTGCCAGATAGTCCAGGTGATAAACTTGTG | |
| | ATCGTGAAATTTGTTCAAGACACTTCCAAGTTCTGGTACAAGGCGGATATTTCAAGAG | |
| | AACAAGCCATCGCCATGTTGAAGGACAAGGAGCCGGGCTCATTCATTGTTCGAGACAG | |
| | CCATTCCTTCCGAGGGGCCTATGGCCTGGCCATGAAGGTGGCCACGCCCCCACCTTCA | |
| | GTCCTGCAGCTGAACAAGAAAGCTGGAGATTTGGCCAATGAACTCGTCCGGCACTTTT | |
| | TGATCGAGTGTACCCCGAAGGGAGTGCGGTTGAAAGGGTGCTCGAATGAACCATATTT | |
| | CGTGTACCGCCGGAAGCACCAGGAGCTGCAAGCCATGCAGATGGAGCTGCAGAGCCCT | |
| | GAGTACAAGCTGAGCAAGCTCCGCACCTCGACCATCATGACCGACTACAACCCCAACT | |
| | ACTGCTTTGCTGGCAAGACCTCCTCCATCAGTGACCTGAAGGAGGTGCCGCGGAAAAA | |
| | CATCACCCTCATTCGGGGTCTGGGCCATGGCGCCTTTGGGGAGGTGTATGAAGGCCAG | |
| | GTGTCCGGAATGCCCAACGACCCAAGCCCCCTGCAAGTGGCTGTGAAGACGCTGCCTG | |
| | AAGTGTGCTCTGAACAGGACGAACTGGATTTCCTCATGGAAGCCCTGATCATCAGCAA | |
| | ATTCAACCACCAGAACATTGTTCGCTGCATTGGGGTGAGCCTGCAATCCCTGCCCCGG | |
| | TTCATCCTGCTGGAGCTCATGGCGGGGGGAGACCTCAAGTCCTTCCTCCGAGAGACCC | |
| | GCCCTCGCCCGAGCCAGCCCTCCTCCCTGGCCATGCTGGACCTTCTGCACGTGGCTCG | |
| | GGACATTGCCTGTGGCTGTCAGTATTTGGAGGAAAACCACTTCATCCACCGAGACATT | |
| | GCTGCCAGAAACTGCCTCTTGACCTGTCCAGGCCCTGGAAGAGTGGCCAAGATTGGAG | |
| | ACTTCGGGATGGCCCGAGACATCTACAGGGCGAGCTACTATAGAAAGGGAGGCTGTGC | |
| | CATGCTGCCAGTTAAGTGGATGCCCCCAGAGGCCTTCATGGAAGGAATATTCACTTCT | |
| | AAAACAGACACATGGTCCTTTGGAGTGCTGCTATGGGAAATCTTTTCTCTTGGATATA | |
| | TGCCATACCCCAGCAAAAGCAACCAGGAAGTTCTGGAGTTTGTCACCAGTGGAGGCCG | |
| | GATGGACCCACCCAAGAACTGCCCTGGGCCTGTATACCGGATAATGACTCAGTGCTGG | |
| | CAACATCAGCCTGAAGACAGGCCCAACTTTGCCATCATTTTGGAGAGGATTGAATACT | |
| | GCACCCAGGACCCGGATGTAATCAACACCGCTTTGCCGATAGAATATGGTCCACTTGT | |
| | GGAAGAGGAAGAGAAAGTGCCTGTGAGGCCCAAGGACCCTGAGGGGGTTCCTCCTCTC | |
| | CTGGTCTCTCAACAGGCAAAACGGGAGGAGGAGCGCAGCCCAGCTGCCCCACCACCTC | |
| | TGCCTACCACCTCCTCTGGCAAGGCTGCAAAGAAACCCACAGCTGCAGAGATCTCTGT | |
| | TCGAGTCCCTAGAGGGCCGGCCGTGGAAGGGGGACACGTGAATATGGCATTCTCTCAG | |
| | TCCAACCCTCCTTCGGAGTTGCACAAGGTCCACGGATCCAGAAACAAGCCCACCAGCT | |
| | TGTGGAACCCAACGTACGGCTCCTGGTTTACAGAGAAACCCACCAAAAAGAATAATCC | |
| | TATAGCAAAGAAGGAGCCACACGACAGGGGTAACCTGGGGCTGGAGGGAAGCTGTACT | |
| | GTCCCACCTAACGTTGCAACTGGGAGACTTCCGGGGGCCTCACTGCTCCTAGAGCCCT | |
| | CTTCGCTGACTGCCAATATGAAGGAGGTACCTCTGTTCAGGCTACGTCACTTCCCTTG | |
| | TGGGAATGTCAATTACGGCTACCAGCAACAGGGCTTGCCCTTAGAAGCCGCTACTGCC | |
| | CCTGGAGCTGGTCATTACGAGGATACCATTCTGAAAAGCAAGAATAGCATGAACCAGC | |
| | CTGGGCCCTGA | |
| ALK- | ATGGAGGAAAAGGAAGATAAGCATCAACAGCATAAAATAGAGGATGCTGCTATAACAT | 9 |
| C2orf73 | ATGTAAGTGAAAATGAAGAAATTAAACACGAAGAAAAACCTGGAAAAAGCATACATCA | |
| | TTCAAAATCACATGTTGGAAGAGGACGTATATATTATGCTAAATTCATTAACACAAAT | |
| | GCAAGAACATACAATGAACCATTTCCCTACATAGATCCCAAAAAAGGGCCAGAAATAC | |
| | AGGGTGACTGGTGGTCACATGGTAAAGCACTGGAACCTGTCTTCCTACCACCTTACGA | |
| | CTCTAAGAGCACCCAGAGGAGTGACTTCCAAAAACCATCGTGTCCACTGGTTTTGCCA | |
| | GTCAAACACAGCAAGATGCAAAAGCCTTCTTGTGGAATAGTGTACCGCCGGAAGCACC | |
| | AGGAGCTGCAAGCCATGCAGATGGAGCTGCAGAGCCCTGAGTACAAGCTGAGCAAGCT | |
| | CCGCACCTCGACCATCATGACCGACTACAACCCCAACTACTGCTTTGCTGGCAAGACC | |
| | TCCTCCATCAGTGACCTGAAGGAGGTGCCGCGGAAAAACATCACCCTCATTCGGGGTC | |
| | TGGGCCATGGCGCCTTTGGGGAGGTGTATGAAGGCCAGGTGTCCGGAATGCCCAACGA | |
| | CCCAAGCCCCCTGCAAGTGGCTGTGAAGACGCTGCCTGAAGTGTGCTCTGAACAGGAC | |
| | GAACTGGATTTCCTCATGGAAGCCCTGATCATCAGCAAATTCAACCACCAGAACATTG | |
| | TTCGCTGCATTGGGGTGAGCCTGCAATCCCTGCCCCGGTTCATCCTGCTGGAGCTCAT | |
| | GGCGGGGGGAGACCTCAAGTCCTTCCTCCGAGAGACCCGCCCTCGCCCGAGCCAGCCC | |
| | TCCTCCCTGGCCATGCTGGACCTTCTGCACGTGGCTCGGGACATTGCCTGTGGCTGTC | |
| | AGTATTTGGAGGAAAACCACTTCATCCACCGAGACATTGCTGCCAGAAACTGCCTCTT | |
| | GACCTGTCCAGGCCCTGGAAGAGTGGCCAAGATTGGAGACTTCGGGATGGCCCGAGAC | |
| | ATCTACAGGGCGAGCTACTATAGAAAGGGAGGCTGTGCCATGCTGCCAGTTAAGTGGA | |
| | TGCCCCCAGAGGCCTTCATGGAAGGAATATTCACTTCTAAAACAGACACATGGTCCTT | |
| | TGGAGTGCTGCTATGGGAAATCTTTTCTCTTGGATATATGCCATACCCCAGCAAAAGC | |
| | AACCAGGAAGTTCTGGAGTTTGTCACCAGTGGAGGCCGGATGGACCCACCCAAGAACT | |
| | GCCCTGGGCCTGTATACCGGATAATGACTCAGTGCTGGCAACATCAGCCTGAAGACAG | |
| | GCCCAACTTTGCCATCATTTTGGAGAGGATTGAATACTGCACCCAGGACCCGGATGTA | |
| | ATCAACACCGCTTTGCCGATAGAATATGGTCCACTTGTGGAAGAGGAAGAGAAAGTGC | |
| | CTGTGAGGCCCAAGGACCCTGAGGGGGTTCCTCCTCTCCTGGTCTCTCAACAGGCAAA | |
| | ACGGGAGGAGGAGCGCAGCCCAGCTGCCCCACCACCTCTGCCTACCACCTCCTCTGGC | |
| | AAGGCTGCAAAGAAACCCACAGCTGCAGAGATCTCTGTTCGAGTCCCTAGAGGGCCGG | |
| | CCGTGGAAGGGGGACACGTGAATATGGCATTCTCTCAGTCCAACCCTCCTTCGGAGTT | |
| | GCACAAGGTCCACGGATCCAGAAACAAGCCCACCAGCTTGTGGAACCCAACGTACGGC | |
| | TCCTGGTTTACAGAGAAACCCACCAAAAAGAATAATCCTATAGCAAAGAAGGAGCCAC | |
| | ACGACAGGGGTAACCTGGGGCTGGAGGGAAGCTGTACTGTCCCACCTAACGTTGCAAC | |
| | TGGGAGACTTCCGGGGGCCTCACTGCTCCTAGAGCCCTCTTCGCTGACTGCCAATATG | |
| | AAGGAGGTACCTCTGTTCAGGCTACGTCACTTCCCTTGTGGGAATGTCAATTACGGCT | |
| | ACCAGCAACAGGGCTTGCCCTTAGAAGCCGCTACTGCCCCTGGAGCTGGTCATTACGA | |
| | GGATACCATTCTGAAAAGCAAGAATAGCATGAACCAGCCTGGGCCCTGA | |
| ALK-AZI2 | ATGGATGCACTGGTAGAAGATGATATCTGTATTCTGAATCATGAAAAAGCCCATAAGA | 10 |
| | GAGATACAGTGACTCCAGTTTCAATATATTCAGGAGATGAATCTGTTGCTTCCCATTT | |
| | TGCTCTTGTCACTGCATATGAAGACATCAAAAAACGACTTAAGGATTCAGAGAAAGAG | |
| | AACTCTTTGTTAAAGAAGAGAATAAGATTTTTGGAAGAAAAGCTAATAGCTCGATTTG | |
| | AAGAAGAAACAAGTTCCGTGGGACGAGAACAAGTAAATAAGGCCTATCATGCATATCG | |
| | AGAGGTTTGCATTGATAGAGATAATTTGAAGAGCAAACTGGACAAAATGAATAAAGAC | |
| | AACTCTGAATCTTTGAAAGTATTGAATGAGCAGCTACAATCTAAAGAAGTAGAACTCC | |
| | TCCAGCTGAGGACAGAGGTGGAAACTCAGCAGGTGATGAGGAATTTAAATCCACCTTC | |
| | ATCAAACTGGGAGGTGGAAAAGTTGAGCTGTGACCTGAAGATCCATGGTTTGGAACAA | |
| | GAGCTGGAACTGATGAGGAAAGAATGTAGCGATCTCAAAATAGAACTACAGAAAGCCA | |
| | AACAAACGGATCCATATCAGGAAGACAATCTGAAGAGCAGAGATCTCCAAAAACTAAG | |
| | CATTTCAAGTGATAATATGCAGCATGCATACTGGGAACTGAAGAGAGAAATGTCTAAT | |
| | TTACATCTGGTGACTCAAGTACAAGCTGAACTACTAAGAAAACTGAAAACCTCAACTG | |
| | CAATCAAGAAAGTGTACCGCCGGAAGCACCAGGAGCTGCAAGCCATGCAGATGGAGCT | |
| | GCAGAGCCCTGAGTACAAGCTGAGCAAGCTCCGCACCTCGACCATCATGACCGACTAC | |
| | AACCCCAACTACTGCTTTGCTGGCAAGACCTCCTCCATCAGTGACCTGAAGGAGGTGC | |
| | CGCGGAAAAACATCACCCTCATTCGGGGTCTGGGCCATGGCGCCTTTGGGGAGGTGTA | |
| | TGAAGGCCAGGTGTCCGGAATGCCCAACGACCCAAGCCCCCTGCAAGTGGCTGTGAAG | |
| | ACGCTGCCTGAAGTGTGCTCTGAACAGGACGAACTGGATTTCCTCATGGAAGCCCTGA | |
| | TCATCAGCAAATTCAACCACCAGAACATTGTTCGCTGCATTGGGGTGAGCCTGCAATC | |
| | CCTGCCCCGGTTCATCCTGCTGGAGCTCATGGCGGGGGGAGACCTCAAGTCCTTCCTC | |
| | CGAGAGACCCGCCCTCGCCCGAGCCAGCCCTCCTCCCTGGCCATGCTGGACCTTCTGC | |
| | ACGTGGCTCGGGACATTGCCTGTGGCTGTCAGTATTTGGAGGAAAACCACTTCATCCA | |
| | CCGAGACATTGCTGCCAGAAACTGCCTCTTGACCTGTCCAGGCCCTGGAAGAGTGGCC | |
| | AAGATTGGAGACTTCGGGATGGCCCGAGACATCTACAGGGCGAGCTACTATAGAAAGG | |
| | GAGGCTGTGCCATGCTGCCAGTTAAGTGGATGCCCCCAGAGGCCTTCATGGAAGGAAT | |
| | ATTCACTTCTAAAACAGACACATGGTCCTTTGGAGTGCTGCTATGGGAAATCTTTTCT | |
| | CTTGGATATATGCCATACCCCAGCAAAAGCAACCAGGAAGTTCTGGAGTTTGTCACCA | |
| | GTGGAGGCCGGATGGACCCACCCAAGAACTGCCCTGGGCCTGTATACCGGATAATGAC | |
| | TCAGTGCTGGCAACATCAGCCTGAAGACAGGCCCAACTTTGCCATCATTTTGGAGAGG | |
| | ATTGAATACTGCACCCAGGACCCGGATGTAATCAACACCGCTTTGCCGATAGAATATG | |
| | GTCCACTTGTGGAAGAGGAAGAGAAAGTGCCTGTGAGGCCCAAGGACCCTGAGGGGGT | |
| | TCCTCCTCTCCTGGTCTCTCAACAGGCAAAACGGGAGGAGGAGCGCAGCCCAGCTGCC | |
| | CCACCACCTCTGCCTACCACCTCCTCTGGCAAGGCTGCAAAGAAACCCACAGCTGCAG | |
| | AGATCTCTGTTCGAGTCCCTAGAGGGCCGGCCGTGGAAGGGGGACACGTGAATATGGC | |
| | ATTCTCTCAGTCCAACCCTCCTTCGGAGTTGCACAAGGTCCACGGATCCAGAAACAAG | |
| | CCCACCAGCTTGTGGAACCCAACGTACGGCTCCTGGTTTACAGAGAAACCCACCAAAA | |
| | AGAATAATCCTATAGCAAAGAAGGAGCCACACGACAGGGGTAACCTGGGGCTGGAGGG | |
| | AAGCTGTACTGTCCCACCTAACGTTGCAACTGGGAGACTTCCGGGGGCCTCACTGCTC | |
| | CTAGAGCCCTCTTCGCTGACTGCCAATATGAAGGAGGTACCTCTGTTCAGGCTACGTC | |
| | ACTTCCCTTGTGGGAATGTCAATTACGGCTACCAGCAACAGGGCTTGCCCTTAGAAGC | |
| | CGCTACTGCCCCTGGAGCTGGTCATTACGAGGATACCATTCTGAAAAGCAAGAATAGC | |
| | ATGAACCAGCCTGGGCCCTGA | |
| ALK- | ATGCGCCTCCACCTGCTCCTGCTGCTCGCGCTGTGCGGTGCAGGCACCACCGCCGCGG | 11 |
| MANBA | AGCTCAGTTACAGCTTGCGTGGCAACTGGAGCATCTGCAATGGGAACGGCTCGCTGGA | |
| | GCTGCCCGGGGCGGTCCCTGGCTGCGTGCACAGCGCCTTGTTCCAGCAGGGCCTGATC | |
| | CAGGATTCTTACTACAGATTTAATGACCTTAACTACAGATGGGTCTCTTTGGATAACT | |
| | GGACCTATAGCAAAGAATTTAAAATCCCCTTTGAAATTAGCAAATGGCAAAAAGTAAA | |
| | TTTGATTCTTGAGGGAGTGGATACGGTTTCAAAAATCCTGTTCAATGAAGTCACTATT | |
| | GGGGAAACAGACAATATGTTCAATAGATATAGCTTTGATATTACCAACGTGGTCAGGG | |
| | ACGTGAACTCCATTGAGCTGCGTTTCCAGTCAGCGGTGTTGTATGCAGCACAGCAGAG | |
| | CAAAGCTCACACTCGCTACCAGGTTCCCCCAGACTGCCCTCCACTTGTGCAGAAGGGT | |
| | GAATGCCATGTCAACTTTGTTCGGAAGGAGCAATGTTCCTTTAGTTGGGACTGGGGGC | |
| | CTTCCTTTCCTACCCAGGGAATCTGGAAAGATGTTAGAATTGAAGCCTATAATATTTG | |
| | TCACCTGAACTACTTCACATTTTCCCCAATATATGATAAGAGTGCCCAGGAGTGGAAT | |
| | CTGGAAATAGAGTCTACATTTGATGTTGTCAGCTCAAAGCCAGTTGGTGGTCAAGTGA | |
| | TCGTAGCCATCCCTAAGTTGCAAACACAACAGACATACAGCATTGAACTTCAACCTGG | |
| | GAAAAGGATTGTTGAGCTATTTGTGAACATTAGCAAGAATATTACTGTAGAAACTTGG | |
| | TGGCCTCATGGACATGGAAACCAGACTGGGTACAACATGACTGTTCTTTTTGAACTGG | |
| | ATGGAGGCTTAAATATTGAAAAATCAGCTAAGGTTTATTTTAGGACAGTGGAACTTAT | |
| | AGAAGAGCCTATAAAAGGGTCTCCTGGTTTGAGTTTCTATTTCAAAATTAATGGATTT | |
| | CCCATATTTCTAAAAGGCTCAAACTGGATCCCAGCAGATTCATTCCAGGACCGAGTAA | |
| | CCTCTGAGTTGTTACGGCTCCTTTTACAGTCTGTTGTGGATGCTAATATGAATACTCT | |
| | TCGGGTTTGGGGAGGAGGAATTTATGAGCAGGATGAATTCTATGAACTCTGTGATGAA | |
| | CTAGGAATAATGGTATGGCAGGATTTTATGTTTGCCTGTGCCCTTTATCCAACTGATC | |
| | AGGGCTTCCTGGATTCAGTGACAGCAGAAGTTGCCTACCAGATCAAGAGACTGAAATC | |
| | TCATCCTTCTATCATCATATGGAGTGGCAATAATGAAAATGAGGAGGCGCTGATGATG | |
| | AATTGGTATCATATCAGTTTCACTGACCGGCCAATCTACATCAAGGACTATGTGACAC | |
| | TCTATGTGAAAAACATCAGAGAGCTCGTACTGGCAGGAGACAAGAGTCGTCCTTTTAT | |
| | TACGTCCAGTCCTACAAATGGGGCTGAAACTGTTGCAGAAGCCTGGGTCTCTCAAAAC | |
| | CCTAATAGCAATTATTTTGGTGATGTACATTTTTATGACTATATCAGTGATTGCTGGA | |
| | ACTGGAAAGTTTTCCCAAAAGCTCGATTTGCATCTGAATATGGATATCAGTCCTGGCC | |
| | GTCCTTCAGTACATTAGAAAAGGTCTCGTCTACAGAGGACTGGTCTTTCAATAGCAAG | |
| | TTTTCACTTCATCGACAACATCACGAAGGTGGTAACAAACAAATGCTTTATCAGGCTG | |
| | GACTTCATTTCAAACTCCCCCAAAGCACAGATCCATTACGCACATTTAAAGATACCAT | |
| | CTACCTTACTCAGGTGATGCAGGCCCAGTGTGTCAAAACAGAAACTGAATTCTACCGC | |
| | CGTAGTCGCAGCGAGATAGTGGATCAGCAAGGGCACACGATGGGGGCACTTTATTGGC | |
| | AGTTGAATGACATCTGGCAAGCTCCTTCCTGGGCTTCTCTTGTGTACCGCCGGAAGCA | |
| | CCAGGAGCTGCAAGCCATGCAGATGGAGCTGCAGAGCCCTGAGTACAAGCTGAGCAAG | |
| | CTCCGCACCTCGACCATCATGACCGACTACAACCCCAACTACTGCTTTGCTGGCAAGA | |
| | CCTCCTCCATCAGTGACCTGAAGGAGGTGCCGCGGAAAAACATCACCCTCATTCGGGG | |
| | TCTGGGCCATGGCGCCTTTGGGGAGGTGTATGAAGGCCAGGTGTCCGGAATGCCCAAC | |
| | GACCCAAGCCCCCTGCAAGTGGCTGTGAAGACGCTGCCTGAAGTGTGCTCTGAACAGG | |
| | ACGAACTGGATTTCCTCATGGAAGCCCTGATCATCAGCAAATTCAACCACCAGAACAT | |
| | TGTTCGCTGCATTGGGGTGAGCCTGCAATCCCTGCCCCGGTTCATCCTGCTGGAGCTC | |
| | ATGGCGGGGGGAGACCTCAAGTCCTTCCTCCGAGAGACCCGCCCTCGCCCGAGCCAGC | |
| | CCTCCTCCCTGGCCATGCTGGACCTTCTGCACGTGGCTCGGGACATTGCCTGTGGCTG | |
| | TCAGTATTTGGAGGAAAACCACTTCATCCACCGAGACATTGCTGCCAGAAACTGCCTC | |
| | TTGACCTGTCCAGGCCCTGGAAGAGTGGCCAAGATTGGAGACTTCGGGATGGCCCGAG | |
| | ACATCTACAGGGCGAGCTACTATAGAAAGGGAGGCTGTGCCATGCTGCCAGITAAGTG | |
| | GATGCCCCCAGAGGCCTTCATGGAAGGAATATTCACTTCTAAAACAGACACATGGTCC | |
| | TTTGGAGTGCTGCTATGGGAAATCTTTTCTCTTGGATATATGCCATACCCCAGCAAAA | |
| | GCAACCAGGAAGTTCTGGAGTTTGTCACCAGTGGAGGCCGGATGGACCCACCCAAGAA | |
| | CTGCCCTGGGCCTGTATACCGGATAATGACTCAGTGCTGGCAACATCAGCCTGAAGAC | |
| | AGGCCCAACTTTGCCATCATTTTGGAGAGGATTGAATACTGCACCCAGGACCCGGATG | |
| | TAATCAACACCGCTTTGCCGATAGAATATGGTCCACTTGTGGAAGAGGAAGAGAAAGT | |
| | GCCTGTGAGGCCCAAGGACCCTGAGGGGGTTCCTCCTCTCCTGGTCTCTCAACAGGCA | |
| | AAACGGGAGGAGGAGCGCAGCCCAGCTGCCCCACCACCTCTGCCTACCACCTCCTCTG | |
| | GCAAGGCTGCAAAGAAACCCACAGCTGCAGAGATCTCTGTTCGAGTCCCTAGAGGGCC | |
| | GGCCGTGGAAGGGGGACACGTGAATATGGCATTCTCTCAGTCCAACCCTCCTTCGGAG | |
| | TTGCACAAGGTCCACGGATCCAGAAACAAGCCCACCAGCTTGTGGAACCCAACGTACG | |
| | GCTCCTGGTTTACAGAGAAACCCACCAAAAAGAATAATCCTATAGCAAAGAAGGAGCC | |
| | ACACGACAGGGGTAACCTGGGGCTGGAGGGAAGCTGTACTGTCCCACCTAACGTTGCA | |
| | ACTGGGAGACTTCCGGGGGCCTCACTGCTCCTAGAGCCCTCTTCGCTGACTGCCAATA | |
| | TGAAGGAGGTACCTCTGTTCAGGCTACGTCACTTCCCTTGTGGGAATGTCAATTACGG | |
| | CTACCAGCAACAGGGCTTGCCCTTAGAAGCCGCTACTGCCCCTGGAGCTGGTCATTAC | |
| | GAGGATACCATTCTGAAAAGCAAGAATAGCATGAACCAGCCTGGGCCCTGA | |
| ALK- | ATGGATTCTTTACCACGGCTGACCAGCGTTTTGACTTTGCTGTTCTCTGGCTTGTGGC | 12 |
| CNTNAP5 | ATTTAGGATTAACAGCGACAAACTACAACTGTGATGATCCACTAGCATCCCTGCTCTC | |
| | TCCAATGGCTTTTTCCAGTTCCTCAGACCTCACTGGCACTCACAGCCCAGCTCAACTC | |
| | AACTGGAGAGTTGTGTACCGCCGGAAGCACCAGGAGCTGCAAGCCATGCAGATGGAGC | |
| | TGCAGAGCCCTGAGTACAAGCTGAGCAAGCTCCGCACCTCGACCATCATGACCGACTA | |
| | CAACCCCAACTACTGCTTTGCTGGCAAGACCTCCTCCATCAGTGACCTGAAGGAGGTG | |
| | CCGCGGAAAAACATCACCCTCATTCGGGGTCTGGGCCATGGCGCCTTTGGGGAGGTGT | |
| | ATGAAGGCCAGGTGTCCGGAATGCCCAACGACCCAAGCCCCCTGCAAGTGGCTGTGAA | |
| | GACGCTGCCTGAAGTGTGCTCTGAACAGGACGAACTGGATTTCCTCATGGAAGCCCTG | |
| | ATCATCAGCAAATTCAACCACCAGAACATTGTTCGCTGCATTGGGGTGAGCCTGCAAT | |
| | CCCTGCCCCGGTTCATCCTGCTGGAGCTCATGGCGGGGGGAGACCTCAAGTCCTTCCT | |
| | CCGAGAGACCCGCCCTCGCCCGAGCCAGCCCTCCTCCCTGGCCATGCTGGACCTTCTG | |
| | CACGTGGCTCGGGACATTGCCTGTGGCTGTCAGTATTTGGAGGAAAACCACTTCATCC | |
| | ACCGAGACATTGCTGCCAGAAACTGCCTCTTGACCTGTCCAGGCCCTGGAAGAGTGGC | |
| | CAAGATTGGAGACTTCGGGATGGCCCGAGACATCTACAGGGCGAGCTACTATAGAAAG | |
| | GGAGGCTGTGCCATGCTGCCAGTTAAGTGGATGCCCCCAGAGGCCTTCATGGAAGGAA | |
| | TATTCACTTCTAAAACAGACACATGGTCCTTTGGAGTGCTGCTATGGGAAATCTTTTC | |
| | TCTTGGATATATGCCATACCCCAGCAAAAGCAACCAGGAAGTTCTGGAGTTTGTCACC | |
| | AGTGGAGGCCGGATGGACCCACCCAAGAACTGCCCTGGGCCTGTATACCGGATAATGA | |
| | CTCAGTGCTGGCAACATCAGCCTGAAGACAGGCCCAACTTTGCCATCATTTTGGAGAG | |
| | GATTGAATACTGCACCCAGGACCCGGATGTAATCAACACCGCTTTGCCGATAGAATAT | |
| | GGTCCACTTGTGGAAGAGGAAGAGAAAGTGCCTGTGAGGCCCAAGGACCCTGAGGGGG | |
| | TTCCTCCTCTCCTGGTCTCTCAACAGGCAAAACGGGAGGAGGAGCGCAGCCCAGCTGC | |
| | CCCACCACCTCTGCCTACCACCTCCTCTGGCAAGGCTGCAAAGAAACCCACAGCTGCA | |
| | GAGATCTCTGTTCGAGTCCCTAGAGGGCCGGCCGTGGAAGGGGGACACGTGAATATGG | |
| | CATTCTCTCAGTCCAACCCTCCTTCGGAGTTGCACAAGGTCCACGGATCCAGAAACAA | |
| | GCCCACCAGCTTGTGGAACCCAACGTACGGCTCCTGGTTTACAGAGAAACCCACCAAA | |
| | AAGAATAATCCTATAGCAAAGAAGGAGCCACACGACAGGGGTAACCTGGGGCTGGAGG | |
| | GAAGCTGTACTGTCCCACCTAACGTTGCAACTGGGAGACTTCCGGGGGCCTCACTGCT | |
| | CCTAGAGCCCTCTTCGCTGACTGCCAATATGAAGGAGGTACCTCTGTTCAGGCTACGT | |
| | CACTTCCCTTGTGGGAATGTCAATTACGGCTACCAGCAACAGGGCTTGCCCTTAGAAG | |
| | CCGCTACTGCCCCTGGAGCTGGTCATTACGAGGATACCATTCTGAAAAGCAAGAATAG | |
| | CATGAACCAGCCTGGGCCCTGA | |
| ALK- | ATGGCGGCCCGACAGGCCGTGGGCAGCGGGGCTCAGGAGACATGCGGTCTGGATCGGA | 14 |
| TANGO6 | TTTTGGAGGCATTGAAGCTGCTGCTGAGCCCGGGAGGTCATAGCTCCTTGGAATCACC | |
| | AACAAACATGCCTTCTCCTTCTCCTGATTATTTTACATGGAATCTCACCTGGATAATG | |
| | AAAGACTCCTTCCCTTTCCTGTCTCATCGCAGCCGATATGGTCTGGAGTGCAGCTTTG | |
| | ACTTCCCCTGTGAGCTGGAGTATTCCCCTCCACTGCATGACCTCAGGAACCAGAGCTG | |
| | GTCCTGGCGCCGCATCCCCTCCGAGGAGGCCTCCCAGATGGACTTGCTGGATGGGCCT | |
| | GGGGCAGAGCGTTCTAAGGAGATGCCCAGAGGCTCCTTTCTCCTTCTCAACACCTCAG | |
| | CTGACTCCAAGCACACCATCCTGAGTCCGTGGATGAGGAGCAGCAGTGAGCACTGCAC | |
| | ACTGGCCGTCTCGGTGCACAGGCACCTGCAGCCCTCTGGAAGGTACATTGCCCAGCTG | |
| | CTGCCCCACAACGAGGCTGCAAGAGAGATCCTCCTGATGCCCACTCCAGGGAAGCATG | |
| | GTTGGACAGTGCTCCAGGGAAGAATCGGGCGTCCAGACAACCCATTTCGAGTGGCCCT | |
| | GGAATACATCTCCAGTGGAAACCGCAGCTTGTCTGCAGTGGACTTCTTTGCCCTGAAG | |
| | AACTGCAGTGAAGGAACATCCCCAGGCTCCAAGATGGCCCTGCAGAGCTCCTTCACTT | |
| | GTTGGAATGGGACAGTCCTCCAGCTTGGGCAGGCCTGTGACTTCCACCAGGACTGTGC | |
| | CCAGGGAGAAGATGAGAGCCAGATGTGCCGGAAACTGCCTGTGGGTTTTTACTGCAAC | |
| | TTTGAAGATGGCTTCTGTGGCTGGACCCAAGGCACACTGTCACCCCACACTCCTCAAT | |
| | GGCAGGTCAGGACCCTAAAGGATGCCCGGTTCCAGGACCACCAAGACCATGCTCTATT | |
| | GCTCAGTACCACTGATGTCCCCGCTTCTGAAAGTGCTACAGTGACCAGTGCTACGTTT | |
| | CCTGCACCGATCAAGAGCTCTCCATGTGAGCTCCGAATGTCCTGGCTCATTCGTGGAG | |
| | TCTTGAGGGGAAACGTGTCCTTGGTGCTAGTGGAGAACAAAACCGGGAAGGAGCAAGG | |
| | CAGGATGGTCTGGCATGTCGCCGCCTATGAAGGCTTGAGCCTGTGGCAGTGGATGGTG | |
| | TTGCCTCTCCTCGATGTGTCTGACAGGTTCTGGCTGCAGATGGTCGCATGGTGGGGAC | |
| | AAGGATCCAGAGCCATCGTGGCTTTTGACAATATCTCCATCAGCCTGGACTGCTACCT | |
| | CACCATTAGCGGAGAGGACAAGATCCTGCAGAATACAGCACCCAAATCAAGAAACCTG | |
| | TTTGAGAGAAACCCAAACAAGGAGCTGAAACCCGGGGAAAATTCACCAAGACAGACCC | |
| | CCATCTTTGACCCTACAGTTCATTGGCTGTTCACCACATGTGGGGCCAGCGGGCCCCA | |
| | TGGCCCCACCCAGGCACAGTGCAACAACGCCTACCAGAACTCCAACCTGAGCGTGGAG | |
| | GTGGGGAGCGAGGGCCCCCTGAAAGGCATCCAGATCTGGAAGGTGCCAGCCACCGACA | |
| | CCTACAGCATCTCGGGCTACGGAGCTGCTGGCGGGAAAGGCGGGAAGAACACCATGAT | |
| | GCGGTCCCACGGCGTGTCTGTGCTGGGCATCTTCAACCTGGAGAAGGATGACATGCTG | |
| | TACATCCTGGTTGGGCAGCAGGGAGAGGACGCCTGCCCCAGTACAAACCAGTTAATCC | |
| | AGAAAGTCTGCATTGGAGAGAACAATGTGATAGAAGAAGAAATCCGTGTGAACAGAAG | |
| | CGTGCATGAGTGGGCAGGAGGCGGAGGAGGAGGGGGTGGAGCCACCTACGTATTTAAG | |
| | ATGAAGGATGGAGTGCCGGTGCCCCTGATCATTGCAGCCGGAGGTGGTGGCAGGGCCT | |
| | ACGGGGCCAAGACAGACACGTTCCACCCAGAGAGACTGGAGAATAACTCCTCGGTTCT | |
| | AGGGCTAAACGGCAATTCCGGAGCCGCAGGTGGTGGAGGTGGCTGGAATGATAACACT | |
| | TCCTTGCTCTGGGCCGGAAAATCTTTGCAGGAGGGTGCCACCGGAGGACATTCCTGCC | |
| | CCCAGGCCATGAAGAAGTGGGGGTGGGAGACAAGAGGGGGTTTCGGAGGGGGTGGAGG | |
| | GGGGTGCTCCTCAGGTGGAGGAGGCGGAGGATATATAGGCGGCAATGCAGCCTCAAAC | |
| | AATGACCCCGAAATGGATGGGGAAGATGGGGTTTCCTTCATCAGTCCACTGGGCATCC | |
| | TGTACACCCCAGCTTTAAAAGTGATGGAAGGCCACGGGGAAGTGAATATTAAGCATTA | |
| | TCTAAACTGCAGTCACTGTGAGGTAGACGAATGTCACATGGACCCTGAAAGCCACAAG | |
| | GTCATCTGCTTCTGTGACCACGGGACGGTGCTGGCTGAGGATGGCGTCTCCTGCATTG | |
| | TGTCACCCACCCCGGAGCCACACCTGCCACTCTCGCTGATCCTCTCTGTGGTGACCTC | |
| | TGCCCTCGTGGCCGCCCTGGTCCTGGCTTTCTCCGGCATCATGATTGTGTACCGCCGG | |
| | AAGCACCAGGAGCTGCAAGCCATGCAGATGGAGCTGCAGAGCCCTGAGTACAAGCTGA | |
| | GCAAGCTCCGCACCTCGACCATCATGACCGACTACAACCCCAACTACTGCTTTGCTGG | |
| | CAAGACCTCCTCCATCAGTGACCTGAAGGAGGTGCCGCGGAAAAACATCACCCTCATT | |
| | CGGGGTCTGGGCCATGGCGCCTTTGGGGAGGTGTATGAAGGCCAGGTGTCCGGAATGC | |
| | CCAACGACCCAAGCCCCCTGCAAGTGGCTGTGAAGACGCTGCCTGAAGTGTGCTCTGA | |
| | ACAGGACGAACTGGATTTCCTCATGGAAGCCCTGATCATCAGCAAATTCAACCACCAG | |
| | AACATTGTTCGCTGCATTGGGGTGAGCCTGCAATCCCTGCCCCGGTTCATCCTGCTGG | |
| | AGCTCATGGCGGGGGGAGACCTCAAGTCCTTCCTCCGAGAGACCCGCCCTCGCCCGAG | |
| | CCAGCCCTCCTCCCTGGCCATGCTGGACCTTCTGCACGTGGCTCGGGACATTGCCTGT | |
| | GGCTGTCAGTATTTGGAGGAAAACCACTTCATCCACCGAGACATTGCTGCCAGAAACT | |
| | GCCTCTTGACCTGTCCAGGCCCTGGAAGAGTGGCCAAGATTGGAGACTTCGGGATGGC | |
| | CCGAGACATCTACAGGGCGAGCTACTATAGAAAGGGAGGCTGTGCCATGCTGCCAGTT | |
| | AAGTGGATGCCCCCAGAGGCCTTCATGGAAGGAATATTCACTTCTAAAACAGACACAT | |
| | GGTCCTTTGGAGTGCTGCTATGGGAAATCTTTTCTCTTGGATATATGCCATACCCCAG | |
| | CAAAAGCAACCAGGAAGTTCTGGAGTTTGTCACCAGTGGAGGCCGGATGGACCCACCC | |
| | AAGAACTGCCCTGGGCCTGTATACCGGATAATGACTCAGTGCTGGCAACATCAGCCTG | |
| | AAGACAGGCCCAACTTTGCCATCATTTTGGAGAGGATTGAATACTGCACCCAGGACCC | |
| | GGATGTAATCAACACCGCTTTGCCGATAGAATATGGTCCACTTGTGGAAGAGGAAGAG | |
| | AAAGTGCCTGTGAGGCCCAAGGACCCTGAGGGGGTTCCTCCTCTCCTGGTCTCTCAAC | |
| | AGGCAAAACGGGAGGAGGAGCGCAGCCCAGCTGCCCCACCACCTCTGCCTACCACCTC | |
| | CTCTGGCAAGGCTGCAAAGAAACCCACAGCTGCAGAGATCTCTGTTCGAGTCCCTAGA | |
| | GGGCCGGCCGTGGAAGGGGGACACGTGAATATGGCATTCTCTCAGTCCAACCCTCCTT | |
| | CGGAGTTGCACAAGGTCCACGGATCCAGAAACAAGCCCACCAGCTTGTGGAACCCAAC | |
| | GTACGGCTCCTGGTTTACAGAGAAACCCACCAAAAAGAATAATCCTATAGCAAAGAAG | |
| | GAGCCACACGACAGGGGTAACCTGGGGCTGGAGGGAAGCTGTACTGTCCCACCTAACG | |
| | TTGCAACTGGGAGACTTCCGGGGGCCTCACTGCTCCTAGAGCCCTCTTCGCTGACTGC | |
| | CAATATGAAGGAGGTACCTCTGTTCAGGCTACGTCACTTCCCTTGTGGGAATGTCAAT | |
| | TACGGCTACCAGCAACAGGGCTTGCCCTTAGAAGCCGCTACTGCCCCTGGAGCTGGTC | |
| | ATTACGAGGATACCATTCTGAAAAGCAAGAATAGCATGAACCAGCCTGGGCCCTGA | |
| ALK-NFIA | ATGTATTCTCCGCTCTGTCTCACCCAGGATGAATTTCATCCTTTCATCGAAGCACTTC | 15 |
| | TGCCCCACGTCCGAGCCTTTGCCTACACATGGTTCAACCTGCAGGCCCGAAAACGAAA | |
| | ATACTTCAAAAAACATGAAAAGCGTATGTCAAAAGAAGAAGAGAGAGCCGTGAAGGAT | |
| | GAATTGCTAAGTGAAAAACCAGAGGTCAAGCAGAAGTGGGCATCTCGACTTCTGGCAA | |
| | AGTTGCGGAAAGATATCCGACCCGAATATCGAGAGGATTTTGTTCTTACAGTTACAGG | |
| | GAAAAAACCTCCATGTTGTGTTCTTTCCAACCCAGACCAGAAAGGCAAGATGCGAAGA | |
| | ATTGACTGCCTCCGCCAGGCAGATAAAGTCTGGAGGTTGGACCTTGTTATGGTGATTT | |
| | TGTTTAAAGGTATTCCGCTGGAAAGTACTGATGGCGAGCGCCTTGTAAAGTCCCCACA | |
| | ATGCTCTAATCCAGGGCTCTGTGTCCAACCCCATCACATAGGGGTTTCTGTTAAGGAA | |
| | CTCGATTTATATTTGGCATACTTTGTGCATGCAGCAGGTCATAGCTCCTTGGAATCAC | |
| | CAACAAACATGCCTTCTCCTTCTCCTGATTATTTTACATGGAATCTCACCTGGATAAT | |
| | GAAAGACTCCTTCCCTTTCCTGTCTCATCGCAGCCGATATGGTCTGGAGTGCAGCTTT | |
| | GACTTCCCCTGTGAGCTGGAGTATTCCCCTCCACTGCATGACCTCAGGAACCAGAGCT | |
| | GGTCCTGGCGCCGCATCCCCTCCGAGGAGGCCTCCCAGATGGACTTGCTGGATGGGCC | |
| | TGGGGCAGAGCGTTCTAAGGAGATGCCCAGAGGCTCCTTTCTCCTTCTCAACACCTCA | |
| | GCTGACTCCAAGCACACCATCCTGAGTCCGTGGATGAGGAGCAGCAGTGAGCACTGCA | |
| | CACTGGCCGTCTCGGTGCACAGGCACCTGCAGCCCTCTGGAAGGTACATTGCCCAGCT | |
| | GCTGCCCCACAACGAGGCTGCAAGAGAGATCCTCCTGATGCCCACTCCAGGGAAGCAT | |
| | GGTTGGACAGTGCTCCAGGGAAGAATCGGGCGTCCAGACAACCCATTTCGAGTGGCCC | |
| | TGGAATACATCTCCAGTGGAAACCGCAGCTTGTCTGCAGTGGACTTCTTTGCCCTGAA | |
| | GAACTGCAGTGAAGGAACATCCCCAGGCTCCAAGATGGCCCTGCAGAGCTCCTTCACT | |
| | TGTTGGAATGGGACAGTCCTCCAGCTTGGGCAGGCCTGTGACTTCCACCAGGACTGTG | |
| | CCCAGGGAGAAGATGAGAGCCAGATGTGCCGGAAACTGCCTGTGGGTTTTTACTGCAA | |
| | CTTTGAAGATGGCTTCTGTGGCTGGACCCAAGGCACACTGTCACCCCACACTCCTCAA | |
| | TGGCAGGTCAGGACCCTAAAGGATGCCCGGTTCCAGGACCACCAAGACCATGCTCTAT | |
| | TGCTCAGTACCACTGATGTCCCCGCTTCTGAAAGTGCTACAGTGACCAGTGCTACGTT | |
| | TCCTGCACCGATCAAGAGCTCTCCATGTGAGCTCCGAATGTCCTGGCTCATTCGTGGA | |
| | GTCTTGAGGGGAAACGTGTCCTTGGTGCTAGTGGAGAACAAAACCGGGAAGGAGCAAG | |
| | GCAGGATGGTCTGGCATGTCGCCGCCTATGAAGGCTTGAGCCTGTGGCAGTGGATGGT | |
| | GTTGCCTCTCCTCGATGTGTCTGACAGGTTCTGGCTGCAGATGGTCGCATGGTGGGGA | |
| | CAAGGATCCAGAGCCATCGTGGCTTTTGACAATATCTCCATCAGCCTGGACTGCTACC | |
| | TCACCATTAGCGGAGAGGACAAGATCCTGCAGAATACAGCACCCAAATCAAGAAACCT | |
| | GTTTGAGAGAAACCCAAACAAGGAGCTGAAACCCGGGGAAAATTCACCAAGACAGACC | |
| | CCCATCTTTGACCCTACAGTTCATTGGCTGTTCACCACATGTGGGGCCAGCGGGCCCC | |
| | ATGGCCCCACCCAGGCACAGTGCAACAACGCCTACCAGAACTCCAACCTGAGCGTGGA | |
| | GGTGGGGAGCGAGGGCCCCCTGAAAGGCATCCAGATCTGGAAGGTGCCAGCCACCGAC | |
| | ACCTACAGCATCTCGGGCTACGGAGCTGCTGGCGGGAAAGGCGGGAAGAACACCATGA | |
| | TGCGGTCCCACGGCGTGTCTGTGCTGGGCATCTTCAACCTGGAGAAGGATGACATGCT | |
| | GTACATCCTGGTTGGGCAGCAGGGAGAGGACGCCTGCCCCAGTACAAACCAGTTAATC | |
| | CAGAAAGTCTGCATTGGAGAGAACAATGTGATAGAAGAAGAAATCCGTGTGAACAGAA | |
| | GCGTGCATGAGTGGGCAGGAGGCGGAGGAGGAGGGGGTGGAGCCACCTACGTATTTAA | |
| | GATGAAGGATGGAGTGCCGGTGCCCCTGATCATTGCAGCCGGAGGTGGTGGCAGGGCC | |
| | TACGGGGCCAAGACAGACACGTTCCACCCAGAGAGACTGGAGAATAACTCCTCGGTTC | |
| | TAGGGCTAAACGGCAATTCCGGAGCCGCAGGTGGTGGAGGTGGCTGGAATGATAACAC | |
| | TTCCTTGCTCTGGGCCGGAAAATCTTTGCAGGAGGGTGCCACCGGAGGACATTCCTGC | |
| | CCCCAGGCCATGAAGAAGTGGGGGTGGGAGACAAGAGGGGGTTTCGGAGGGGGTGGAG | |
| | GGGGGTGCTCCTCAGGTGGAGGAGGCGGAGGATATATAGGCGGCAATGCAGCCTCAAA | |
| | CAATGACCCCGAAATGGATGGGGAAGATGGGGTTTCCTTCATCAGTCCACTGGGCATC | |
| | CTGTACACCCCAGCTTTAAAAGTGATGGAAGGCCACGGGGAAGTGAATATTAAGCATT | |
| | ATCTAAACTGCAGTCACTGTGAGGTAGACGAATGTCACATGGACCCTGAAAGCCACAA | |
| | GGTCATCTGCTTCTGTGACCACGGGACGGTGCTGGCTGAGGATGGCGTCTCCTGCATT | |
| | GTGTCACCCACCCCGGAGCCACACCTGCCACTCTCGCTGATCCTCTCTGTGGTGACCT | |
| | CTGCCCTCGTGGCCGCCCTGGTCCTGGCTTTCTCCGGCATCATGATTGTGTACCGCCG | |
| | GAAGCACCAGGAGCTGCAAGCCATGCAGATGGAGCTGCAGAGCCCTGAGTACAAGCTG | |
| | AGCAAGCTCCGCACCTCGACCATCATGACCGACTACAACCCCAACTACTGCTTTGCTG | |
| | GCAAGACCTCCTCCATCAGTGACCTGAAGGAGGTGCCGCGGAAAAACATCACCCTCAT | |
| | TCGGGGTCTGGGCCATGGCGCCTTTGGGGAGGTGTATGAAGGCCAGGTGTCCGGAATG | |
| | CCCAACGACCCAAGCCCCCTGCAAGTGGCTGTGAAGACGCTGCCTGAAGTGTGCTCTG | |
| | AACAGGACGAACTGGATTTCCTCATGGAAGCCCTGATCATCAGCAAATTCAACCACCA | |
| | GAACATTGTTCGCTGCATTGGGGTGAGCCTGCAATCCCTGCCCCGGTTCATCCTGCTG | |
| | GAGCTCATGGCGGGGGGAGACCTCAAGTCCTTCCTCCGAGAGACCCGCCCTCGCCCGA | |
| | GCCAGCCCTCCTCCCTGGCCATGCTGGACCTTCTGCACGTGGCTCGGGACATTGCCTG | |
| | TGGCTGTCAGTATTTGGAGGAAAACCACTTCATCCACCGAGACATTGCTGCCAGAAAC | |
| | TGCCTCTTGACCTGTCCAGGCCCTGGAAGAGTGGCCAAGATTGGAGACTTCGGGATGG | |
| | CCCGAGACATCTACAGGGCGAGCTACTATAGAAAGGGAGGCTGTGCCATGCTGCCAGT | |
| | TAAGTGGATGCCCCCAGAGGCCTTCATGGAAGGAATATTCACTTCTAAAACAGACACA | |
| | TGGTCCTTTGGAGTGCTGCTATGGGAAATCTTTTCTCTTGGATATATGCCATACCCCA | |
| | GCAAAAGCAACCAGGAAGTTCTGGAGTTTGTCACCAGTGGAGGCCGGATGGACCCACC | |
| | CAAGAACTGCCCTGGGCCTGTATACCGGATAATGACTCAGTGCTGGCAACATCAGCCT | |
| | GAAGACAGGCCCAACTTTGCCATCATTTTGGAGAGGATTGAATACTGCACCCAGGACC | |
| | CGGATGTAATCAACACCGCTTTGCCGATAGAATATGGTCCACTTGTGGAAGAGGAAGA | |
| | GAAAGTGCCTGTGAGGCCCAAGGACCCTGAGGGGGTTCCTCCTCTCCTGGTCTCTCAA | |
| | CAGGCAAAACGGGAGGAGGAGCGCAGCCCAGCTGCCCCACCACCTCTGCCTACCACCT | |
| | CCTCTGGCAAGGCTGCAAAGAAACCCACAGCTGCAGAGATCTCTGTTCGAGTCCCTAG | |
| | AGGGCCGGCCGTGGAAGGGGGACACGTGAATATGGCATTCTCTCAGTCCAACCCTCCT | |
| | TCGGAGTTGCACAAGGTCCACGGATCCAGAAACAAGCCCACCAGCTTGTGGAACCCAA | |
| | CGTACGGCTCCTGGTTTACAGAGAAACCCACCAAAAAGAATAATCCTATAGCAAAGAA | |
| | GGAGCCACACGACAGGGGTAACCTGGGGCTGGAGGGAAGCTGTACTGTCCCACCTAAC | |
| | GTTGCAACTGGGAGACTTCCGGGGGCCTCACTGCTCCTAGAGCCCTCTTCGCTGACTG | |
| | CCAATATGAAGGAGGTACCTCTGTTCAGGCTACGTCACTTCCCTTGTGGGAATGTCAA | |
| | TTACGGCTACCAGCAACAGGGCTTGCCCTTAGAAGCCGCTACTGCCCCTGGAGCTGGT | |
| | CATTACGAGGATACCATTCTGAAAAGCAAGAATAGCATGAACCAGCCTGGGCCCTGA | |
| ALK- | ATGGAGGAGGAGGGTGGCAGCAGCGGCGGCGCCGCGGGGACCAGCGCGGACGGCGGCG | 16 |
| RPS6KA5 | ACGGAGGAGAGCAGCTCCTCACTGTCAAGCACGAGCTGCGGACTGACCATGCTCTATT | |
| | GCTCAGTACCACTGATGTCCCCGCTTCTGAAAGTGCTACAGTGACCAGTGCTACGTTT | |
| | CCTGCACCGATCAAGAGCTCTCCATGTGAGCTCCGAATGTCCTGGCTCATTCGTGGAG | |
| | TCTTGAGGGGAAACGTGTCCTTGGTGCTAGTGGAGAACAAAACCGGGAAGGAGCAAGG | |
| | CAGGATGGTCTGGCATGTCGCCGCCTATGAAGGCTTGAGCCTGTGGCAGTGGATGGTG | |
| | TTGCCTCTCCTCGATGTGTCTGACAGGTTCTGGCTGCAGATGGTCGCATGGTGGGGAC | |
| | AAGGATCCAGAGCCATCGTGGCTTTTGACAATATCTCCATCAGCCTGGACTGCTACCT | |
| | CACCATTAGCGGAGAGGACAAGATCCTGCAGAATACAGCACCCAAATCAAGAAACCTG | |
| | TTTGAGAGAAACCCAAACAAGGAGCTGAAACCCGGGGAAAATTCACCAAGACAGACCC | |
| | CCATCTTTGACCCTACAGTTCATTGGCTGTTCACCACATGTGGGGCCAGCGGGCCCCA | |
| | TGGCCCCACCCAGGCACAGTGCAACAACGCCTACCAGAACTCCAACCTGAGCGTGGAG | |
| | GTGGGGAGCGAGGGCCCCCTGAAAGGCATCCAGATCTGGAAGGTGCCAGCCACCGACA | |
| | CCTACAGCATCTCGGGCTACGGAGCTGCTGGCGGGAAAGGCGGGAAGAACACCATGAT | |
| | GCGGTCCCACGGCGTGTCTGTGCTGGGCATCTTCAACCTGGAGAAGGATGACATGCTG | |
| | TACATCCTGGTTGGGCAGCAGGGAGAGGACGCCTGCCCCAGTACAAACCAGTTAATCC | |
| | AGAAAGTCTGCATTGGAGAGAACAATGTGATAGAAGAAGAAATCCGTGTGAACAGAAG | |
| | CGTGCATGAGTGGGCAGGAGGCGGAGGAGGAGGGGGTGGAGCCACCTACGTATTTAAG | |
| | ATGAAGGATGGAGTGCCGGTGCCCCTGATCATTGCAGCCGGAGGTGGTGGCAGGGCCT | |
| | ACGGGGCCAAGACAGACACGTTCCACCCAGAGAGACTGGAGAATAACTCCTCGGTTCT | |
| | AGGGCTAAACGGCAATTCCGGAGCCGCAGGTGGTGGAGGTGGCTGGAATGATAACACT | |
| | TCCTTGCTCTGGGCCGGAAAATCTTTGCAGGAGGGTGCCACCGGAGGACATTCCTGCC | |
| | CCCAGGCCATGAAGAAGTGGGGGTGGGAGACAAGAGGGGGTTTCGGAGGGGGTGGAGG | |
| | GGGGTGCTCCTCAGGTGGAGGAGGCGGAGGATATATAGGCGGCAATGCAGCCTCAAAC | |
| | AATGACCCCGAAATGGATGGGGAAGATGGGGTTTCCTTCATCAGTCCACTGGGCATCC | |
| | TGTACACCCCAGCTTTAAAAGTGATGGAAGGCCACGGGGAAGTGAATATTAAGCATTA | |
| | TCTAAACTGCAGTCACTGTGAGGTAGACGAATGTCACATGGACCCTGAAAGCCACAAG | |
| | GTCATCTGCTTCTGTGACCACGGGACGGTGCTGGCTGAGGATGGCGTCTCCTGCATTG | |
| | TGTCACCCACCCCGGAGCCACACCTGCCACTCTCGCTGATCCTCTCTGTGGTGACCTC | |
| | TGCCCTCGTGGCCGCCCTGGTCCTGGCTTTCTCCGGCATCATGATTGTGTACCGCCGG | |
| | AAGCACCAGGAGCTGCAAGCCATGCAGATGGAGCTGCAGAGCCCTGAGTACAAGCTGA | |
| | GCAAGCTCCGCACCTCGACCATCATGACCGACTACAACCCCAACTACTGCTTTGCTGG | |
| | CAAGACCTCCTCCATCAGTGACCTGAAGGAGGTGCCGCGGAAAAACATCACCCTCATT | |
| | CGGGGTCTGGGCCATGGCGCCTTTGGGGAGGTGTATGAAGGCCAGGTGTCCGGAATGC | |
| | CCAACGACCCAAGCCCCCTGCAAGTGGCTGTGAAGACGCTGCCTGAAGTGTGCTCTGA | |
| | ACAGGACGAACTGGATTTCCTCATGGAAGCCCTGATCATCAGCAAATTCAACCACCAG | |
| | AACATTGTTCGCTGCATTGGGGTGAGCCTGCAATCCCTGCCCCGGTTCATCCTGCTGG | |
| | AGCTCATGGCGGGGGGAGACCTCAAGTCCTTCCTCCGAGAGACCCGCCCTCGCCCGAG | |
| | CCAGCCCTCCTCCCTGGCCATGCTGGACCTTCTGCACGTGGCTCGGGACATTGCCTGT | |
| | GGCTGTCAGTATTTGGAGGAAAACCACTTCATCCACCGAGACATTGCTGCCAGAAACT | |
| | GCCTCTTGACCTGTCCAGGCCCTGGAAGAGTGGCCAAGATTGGAGACTTCGGGATGGC | |
| | CCGAGACATCTACAGGGCGAGCTACTATAGAAAGGGAGGCTGTGCCATGCTGCCAGTT | |
| | AAGTGGATGCCCCCAGAGGCCTTCATGGAAGGAATATTCACTTCTAAAACAGACACAT | |
| | GGTCCTTTGGAGTGCTGCTATGGGAAATCTTTTCTCTTGGATATATGCCATACCCCAG | |
| | CAAAAGCAACCAGGAAGTTCTGGAGTTTGTCACCAGTGGAGGCCGGATGGACCCACCC | |
| | AAGAACTGCCCTGGGCCTGTATACCGGATAATGACTCAGTGCTGGCAACATCAGCCTG | |
| | AAGACAGGCCCAACTTTGCCATCATTTTGGAGAGGATTGAATACTGCACCCAGGACCC | |
| | GGATGTAATCAACACCGCTTTGCCGATAGAATATGGTCCACTTGTGGAAGAGGAAGAG | |
| | AAAGTGCCTGTGAGGCCCAAGGACCCTGAGGGGGTTCCTCCTCTCCTGGTCTCTCAAC | |
| | AGGCAAAACGGGAGGAGGAGCGCAGCCCAGCTGCCCCACCACCTCTGCCTACCACCTC | |
| | CTCTGGCAAGGCTGCAAAGAAACCCACAGCTGCAGAGATCTCTGTTCGAGTCCCTAGA | |
| | GGGCCGGCCGTGGAAGGGGGACACGTGAATATGGCATTCTCTCAGTCCAACCCTCCTT | |
| | CGGAGTTGCACAAGGTCCACGGATCCAGAAACAAGCCCACCAGCTTGTGGAACCCAAC | |
| | GTACGGCTCCTGGTTTACAGAGAAACCCACCAAAAAGAATAATCCTATAGCAAAGAAG | |
| | GAGCCACACGACAGGGGTAACCTGGGGCTGGAGGGAAGCTGTACTGTCCCACCTAACG | |
| | TTGCAACTGGGAGACTTCCGGGGGCCTCACTGCTCCTAGAGCCCTCTTCGCTGACTGC | |
| | CAATATGAAGGAGGTACCTCTGTTCAGGCTACGTCACTTCCCTTGTGGGAATGTCAAT | |
| | TACGGCTACCAGCAACAGGGCTTGCCCTTAGAAGCCGCTACTGCCCCTGGAGCTGGTC | |
| | ATTACGAGGATACCATTCTGAAAAGCAAGAATAGCATGAACCAGCCTGGGCCCTGA | |
| ALK-TG | ATGGCCCTGGTCCTGGAGATCTTCACCCTGCTGGCCTCCATCTGCTGGGTGTCGGCCA | 17 |
| | ATATCTTCGAGTACCAGGTGGATGCCCAGCCCCTTCGTCCCTGTGAGCTGCAGAGGGA | |
| | AACGGCCTTTCTGAAGCAAGCAGACTACGTGCCCCAGTGTGCAGAGGATGGCAGCTTC | |
| | CAGACTGTCCAGTGCCAGAACGACGGCCGCTCCTGCTGGTGTGTGGGTGCCAACGGCA | |
| | GTGAAGTGCTGGGCAGCAGGCAGCCAGGACGGCCTGTGGCTTGTCTGTCATTTTGTCA | |
| | GCTACAGAAACAGCAGATCTTACTGAGTGGCTACATTAACAGCACAGACACCTCCTAC | |
| | CTCCCTCAGTGTCAGGATTCAGGGGACTACGCGCCTGTTCAGTGTGATGTGCAGCAGG | |
| | TCCAGTGCTGGTGTGTGGACGCAGAGGGGATGGAGGTGTATGGGACCCGCCAGCTGGG | |
| | GAGGCCAAAGCGATGTCCAAGGAGCTGTGAAATAAGAAATCGTCGTCTTCTCCACGGG | |
| | GTGGGAGATAAGTCACCACCCCAGTGTTCTGCGGAGGGAGAGTTTATGCCTGTCCAGT | |
| | GCAAATTTGTCAACACCACAGACATGATGATTTTTGATCTGGTCCACAGCTACAACAG | |
| | GTTTCCAGATGCATTTGTGACCTTCAGTTCCTTCCAGAGGAGGTTCCCTGAGGTATCT | |
| | GGGTATTGCCACTGTGCTGACAGCCAAGGGCGGGAACTGGCTGAGACAGGTTTGGAGT | |
| | TGTTACTGGATGAAATTTATGACACCATTTTTGCTGGCCTGGACCTTCCTTCCACCTT | |
| | CACTGAAACCACCCTGTACCGGATACTGCAGAGACGGTTCCTCGCAGTTCAATCAGTC | |
| | ATCTCTGGCAGATTCCGATGCCCCACAAAATGTGAAGTGGAGCGGTTTACAGCAACCA | |
| | GCTTTGGTCACCCCTATGTTCCAAGCTGCCGCCGAAATGGCGACTATCAGGCGGTGCA | |
| | GTGCCAGACGGAAGGGCCCTGCTGGTGTGTGGACGCCCAGGGGAAGGAAATGCATGGA | |
| | ACCCGGCAGCAAGGGGAGCCGCCATCTTGTGCTGAAGGCCAATCTTGTGCCTCCGAAA | |
| | GGCAGCAGGCCTTGTCCAGACTCTACTTTGGGACCTCAGGCTACTTCAGCCAGCACGA | |
| | CCTGTTCTCTTCCCCAGAGAAAAGATGGGCCTCTCCAAGAGTAGCCAGATTTGCCACA | |
| | TCCTGCCCACCCACGATCAAGGAGCTCTTTGTGGACTCTGGGCTTCTCCGCCCAATGG | |
| | TGGAGGGACAGAGCCAACAGTTTTCTGTCTCAGAAAATCTTCTCAAAGAAGCCATCCG | |
| | AGCAATTTTTCCCTCCCGAGGGCTGGCTCGTCTTGCCCTTCAGTTTACCACCAACCCA | |
| | AAGAGACTCCAGCAAAACCTTTTTGGAGGGAAATTTTTGGTGAATGTTGGCCAGTTTA | |
| | ACTTGTCTGGAGCCCTTGGCACAAGAGGCACATTTAACTTCAGTCAATTTTTCCAGCA | |
| | ACTTGGTCTTGCAAGCTTCTTGAATGGAGGGAGACAAGAAGATTTGGCCAAGCCACTC | |
| | TCTGTGGGATTAGATTCAAATTCTTCCACAGGAACCCCTGAAGCTGCTAAGAAGGATG | |
| | GTACTATGAATAAGCCAACTGTGGGCAGCTTTGGCTTTGAAATTAACCTACAAGAGAA | |
| | CCAAAATGCCCTCAAATTCCTTGCTTCTCTCCTGGAGCTTCCAGAATTCCTTCTCTTC | |
| | TTGCAACATGCTATCTCTGTGCCAGAAGATGTGGCAAGAGATTTAGGTGATGTGATGG | |
| | AAACGGTACTCAGCTCCCAGACCTGTGAGCAGACACCTGAAAGGCTATTTGTCCCATC | |
| | ATGCACGACAGAAGGAAGCTATGAGGATGTCCAATGCTTTTCCGGAGAGTGCTGGTGT | |
| | GTGAATTCCTGGGGCAAAGAGCTTCCAGGCTCAAGAGTCAGAGGTGGACAGCCAAGGT | |
| | GCCCCACAGACTGTGAAAAGCAAAGGGCTCGCATGCAAAGCCTCATGGGCAGCCAGCC | |
| | TGCTGGCTCCACCTTGTTTGTCCCTGCTTGTACTAGTGAGGGACATTTCCTGCCTGTC | |
| | CAGTGCTTCAACTCAGAGTGCTACTGTGTTGATGCTGAGGGTCAGGCCATTCCTGGAA | |
| | CTCGAAGTGCAATAGGGAAGCCCAAGAAATGCCCCACGCCCTGTCAATTACAGTCTGA | |
| | GCAAGCTTTCCTCAGGACGGTGCAGGCCCTGCTCTCTAACTCCAGCATGCTACCCACC | |
| | CTTTCCGACACCTACATCCCACAGTGCAGCACCGATGGGCAGTGGAGACAAGTGCAAT | |
| | GCAATGGGCCTCCTGAGCAGGTCTTCGAGTTGTACCAACGATGGGAGGCTCAGAACAA | |
| | GGGCCAGGATCTGACGCCTGCCAAGCTGCTAGTGAAGATCATGAGCTACAGAGAAGCA | |
| | GCTTCCGGAAACTTCAGTCTCTTTATTCAAAGTCTGTATGAGGCTGGCCAGCAAGATG | |
| | TCTTCCCGGTGCTGTCACAATACCCTTCTCTGCAAGATGTCCCACTAGCAGCACTGGA | |
| | AGGGAAACGGCCCCAGCCCAGGGAGAATATCCTCCTGGAGCCCTACCTCTTCTGGCAG | |
| | ATCTTAAATGGCCAACTCAGCCAATACCCGGGGTCCTACTCAGACTTCAGCACTCCTT | |
| | TGGCACATTTTGATCTTCGGAACTGCTGGTGTGTGGATGAGGCTGGCCAAGAACTGGA | |
| | AGGAATGCGGTCTGAGCCAAGCAAGCTCCCAACATTGTCACCCACCCCGGAGCCACAC | |
| | CTGCCACTCTCGCTGATCCTCTCTGTGGTGACCTCTGCCCTCGTGGCCGCCCTGGTCC | |
| | TGGCTTTCTCCGGCATCATGATTGTGTACCGCCGGAAGCACCAGGAGCTGCAAGCCAT | |
| | GCAGATGGAGCTGCAGAGCCCTGAGTACAAGCTGAGCAAGCTCCGCACCTCGACCATC | |
| | ATGACCGACTACAACCCCAACTACTGCTTTGCTGGCAAGACCTCCTCCATCAGTGACC | |
| | TGAAGGAGGTGCCGCGGAAAAACATCACCCTCATTCGGGGTCTGGGCCATGGCGCCTT | |
| | TGGGGAGGTGTATGAAGGCCAGGTGTCCGGAATGCCCAACGACCCAAGCCCCCTGCAA | |
| | GTGGCTGTGAAGACGCTGCCTGAAGTGTGCTCTGAACAGGACGAACTGGATTTCCTCA | |
| | TGGAAGCCCTGATCATCAGCAAATTCAACCACCAGAACATTGTTCGCTGCATTGGGGT | |
| | GAGCCTGCAATCCCTGCCCCGGTTCATCCTGCTGGAGCTCATGGCGGGGGGAGACCTC | |
| | AAGTCCTTCCTCCGAGAGACCCGCCCTCGCCCGAGCCAGCCCTCCTCCCTGGCCATGC | |
| | TGGACCTTCTGCACGTGGCTCGGGACATTGCCTGTGGCTGTCAGTATTTGGAGGAAAA | |
| | CCACTTCATCCACCGAGACATTGCTGCCAGAAACTGCCTCTTGACCTGTCCAGGCCCT | |
| | GGAAGAGTGGCCAAGATTGGAGACTTCGGGATGGCCCGAGACATCTACAGGGCGAGCT | |
| | ACTATAGAAAGGGAGGCTGTGCCATGCTGCCAGTTAAGTGGATGCCCCCAGAGGCCTT | |
| | CATGGAAGGAATATTCACTTCTAAAACAGACACATGGTCCTTTGGAGTGCTGCTATGG | |
| | GAAATCTTTTCTCTTGGATATATGCCATACCCCAGCAAAAGCAACCAGGAAGTTCTGG | |
| | AGTTTGTCACCAGTGGAGGCCGGATGGACCCACCCAAGAACTGCCCTGGGCCTGTATA | |
| | CCGGATAATGACTCAGTGCTGGCAACATCAGCCTGAAGACAGGCCCAACTTTGCCATC | |
| | ATTTTGGAGAGGATTGAATACTGCACCCAGGACCCGGATGTAATCAACACCGCTTTGC | |
| | CGATAGAATATGGTCCACTTGTGGAAGAGGAAGAGAAAGTGCCTGTGAGGCCCAAGGA | |
| | CCCTGAGGGGGTTCCTCCTCTCCTGGTCTCTCAACAGGCAAAACGGGAGGAGGAGCGC | |
| | AGCCCAGCTGCCCCACCACCTCTGCCTACCACCTCCTCTGGCAAGGCTGCAAAGAAAC | |
| | CCACAGCTGCAGAGATCTCTGTTCGAGTCCCTAGAGGGCCGGCCGTGGAAGGGGGACA | |
| | CGTGAATATGGCATTCTCTCAGTCCAACCCTCCTTCGGAGTTGCACAAGGTCCACGGA | |
| | TCCAGAAACAAGCCCACCAGCTTGTGGAACCCAACGTACGGCTCCTGGTTTACAGAGA | |
| | AACCCACCAAAAAGAATAATCCTATAGCAAAGAAGGAGCCACACGACAGGGGTAACCT | |
| | GGGGCTGGAGGGAAGCTGTACTGTCCCACCTAACGTTGCAACTGGGAGACTTCCGGGG | |
| | GCCTCACTGCTCCTAGAGCCCTCTTCGCTGACTGCCAATATGAAGGAGGTACCTCTGT | |
| | TCAGGCTACGTCACTTCCCTTGTGGGAATGTCAATTACGGCTACCAGCAACAGGGCTT | |
| | GCCCTTAGAAGCCGCTACTGCCCCTGGAGCTGGTCATTACGAGGATACCATTCTGAAA | |
| | AGCAAGAATAGCATGAACCAGCCTGGGCCCTGA | |
| ALK- | ATGGGGACTCCTGCTTCTGGAAGGAAAAGAACACCTGTGAAAGACCGATTTTCTGCAG | 18 |
| LRRFIP2 | AAGATGAAGCTTTGAGTAACATTGCCAGAGAGGCAGAGGCAAGGCTGGCAGCAAAACG | |
| | GGCTGCCCGGGCAGAAGCAAGAGATATACGCATGAGAGAACTGGAACGACAACAAAAA | |
| | GAGTACTCTCTTCATTCCTTTGATCGGAAGTGGGGACAGATTCAGAAGTGGCTGGAAG | |
| | ATTCGGAAAGGGCCAGGTATTCCCACCGGTCCAGTCACCATCGTCCTTATCTGGGAGT | |
| | TGAGGATGCATTGTCCATTCGAAGTGTTGGCAGTCACAGGTATGATATGTTCAAGGAT | |
| | AGATCATCAAGACTTTCATCATTAAATCATTCTTACAGTCACTCTCATGGAATGAAGA | |
| | AGAGGTCTTCTGATTCTCATAAAGACCTACTGAGTGGCCTCTACTTTGACCAGAGAAA | |
| | CTATAGCAGTCTTAGACATAGCAAACCCACCTCTGCCTACTACACTCGGCAGTCTTCT | |
| | TCCCTGTACAGTGACCCTCTGGCAACATATAAGAGTGACAGGGCCTCTCCTACTGCAA | |
| | ATTCTGGTCTGCTGAGAAGTGCCAGTCTGGCATCATTGTACAATGGTGGATTATATAA | |
| | CCCTTATGGTCCTCGAACTCCATCTGAATGCAGTTATTATTCATCAAGAATAAGTTCA | |
| | GCCCGAAGCAGTCCAGGGTTTACCAACGATGACACTGCAAGCATTGTGTCTTCTGATC | |
| | GTGCCAGTCGTGGACGAAGGGAGAGTGTGGTATCTGCCGCTGATTATTTCAGTCGCTC | |
| | CAATCGTAGGGGAAGTGTTGTCTCTGAGGTGGATGATATCAGTATCCCAGATTTGTCC | |
| | AGCTTGGATGAAAAATCTGACAAACAGTATGCTGAAAATTATACAAGACCTTCATCTC | |
| | GAAATTCTGCCTCAGCAACAACCCCTCTAAGTGGAAACTCATCCAGACGAGGAAGTGG | |
| | GGACACCAGCAGCTTAATAGATCCAGACACTTCATTAAGTGAATTGCGGGATATCTAT | |
| | GACCTTAAGGACCAGATACAGGATGTAGAAGGGAGATACATGCAGGGGCTTAAAGAAC | |
| | TAAAGGAATCTTTGTCTGAAGTGGAAGAAAAATACAAGAAAGCCATGGTTTCCAATGC | |
| | ACAGTTAGACAATGAGAAGAACAATTTGATCTACCAAGTAGACACACTCAAGGATGTT | |
| | ATTGAAGAGCAGGAGGAACAGATGGCAGAATTTTATAGAGAAAATGAAGAAAAATCAA | |
| | AGGAGTTAGAAAGGCAGAAACATATGTGTAGTGTGCTGCAGCATAAGATGGAAGAACT | |
| | TAAAGAAGGCCTGCGGCAAAGAGATGAGCTTATTGAGGAGAAGCAGCGCATGCAGCAG | |
| | AAAATAGACACCATGACAAAAGAGGTGTTTGACCTCCAGGAGACACTTCTTTGGAAAG | |
| | ATAAAAAAATTGGGGCCCTAGAGAAACAGAAAGAATACATTGCCTGCCTTAGGAATGA | |
| | GCGAGATATGCTCAGAGAGGAGCTGGCTGACCTGCAGGAGACAGTGAAGACGGGAGAG | |
| | AAACATGGCTTAGTTATAATCCCCGATGGCACTCCCAATGGTGATGTCAGTCATGAAC | |
| | CAGTGGCTGGAGCCATCACTGTTGTGTCTCAGGAAGCTGCTCAGGTCTTGGAGTCAGC | |
| | AGGAGAAGGGCCATTAGATGTAAGGCTACGAAAACTTGCTGGAGAGAAGGAAGAACTA | |
| | CTGTCACAGATTAGAAAACTGAAGCTTCAGTTAGAGGAGGAACGACAGAAATGCTCCA | |
| | GGAATGATGGCACAGTGGGTGACCTGGCAGGACTGCAGAATGGCTCAGACTTGCAGTT | |
| | CATCGAAATGCAGATGTACCGCCGGAAGCACCAGGAGCTGCAAGCCATGCAGATGGAG | |
| | CTGCAGAGCCCTGAGTACAAGCTGAGCAAGCTCCGCACCTCGACCATCATGACCGACT | |
| | ACAACCCCAACTACTGCTTTGCTGGCAAGACCTCCTCCATCAGTGACCTGAAGGAGGT | |
| | GCCGCGGAAAAACATCACCCTCATTCGGGGTCTGGGCCATGGCGCCTTTGGGGAGGTG | |
| | TATGAAGGCCAGGTGTCCGGAATGCCCAACGACCCAAGCCCCCTGCAAGTGGCTGTGA | |
| | AGACGCTGCCTGAAGTGTGCTCTGAACAGGACGAACTGGATTTCCTCATGGAAGCCCT | |
| | GATCATCAGCAAATTCAACCACCAGAACATTGTTCGCTGCATTGGGGTGAGCCTGCAA | |
| | TCCCTGCCCCGGTTCATCCTGCTGGAGCTCATGGCGGGGGGAGACCTCAAGTCCTTCC | |
| | TCCGAGAGACCCGCCCTCGCCCGAGCCAGCCCTCCTCCCTGGCCATGCTGGACCTTCT | |
| | GCACGTGGCTCGGGACATTGCCTGTGGCTGTCAGTATTTGGAGGAAAACCACTTCATC | |
| | CACCGAGACATTGCTGCCAGAAACTGCCTCTTGACCTGTCCAGGCCCTGGAAGAGTGG | |
| | CCAAGATTGGAGACTTCGGGATGGCCCGAGACATCTACAGGGCGAGCTACTATAGAAA | |
| | GGGAGGCTGTGCCATGCTGCCAGTTAAGTGGATGCCCCCAGAGGCCTTCATGGAAGGA | |
| | ATATTCACTTCTAAAACAGACACATGGTCCTTTGGAGTGCTGCTATGGGAAATCTTTT | |
| | CTCTTGGATATATGCCATACCCCAGCAAAAGCAACCAGGAAGTTCTGGAGTTTGTCAC | |
| | CAGTGGAGGCCGGATGGACCCACCCAAGAACTGCCCTGGGCCTGTATACCGGATAATG | |
| | ACTCAGTGCTGGCAACATCAGCCTGAAGACAGGCCCAACTTTGCCATCATTTTGGAGA | |
| | GGATTGAATACTGCACCCAGGACCCGGATGTAATCAACACCGCTTTGCCGATAGAATA | |
| | TGGTCCACTTGTGGAAGAGGAAGAGAAAGTGCCTGTGAGGCCCAAGGACCCTGAGGGG | |
| | GTTCCTCCTCTCCTGGTCTCTCAACAGGCAAAACGGGAGGAGGAGCGCAGCCCAGCTG | |
| | CCCCACCACCTCTGCCTACCACCTCCTCTGGCAAGGCTGCAAAGAAACCCACAGCTGC | |
| | AGAGATCTCTGTTCGAGTCCCTAGAGGGCCGGCCGTGGAAGGGGGACACGTGAATATG | |
| | GCATTCTCTCAGTCCAACCCTCCTTCGGAGTTGCACAAGGTCCACGGATCCAGAAACA | |
| | AGCCCACCAGCTTGTGGAACCCAACGTACGGCTCCTGGTTTACAGAGAAACCCACCAA | |
| | AAAGAATAATCCTATAGCAAAGAAGGAGCCACACGACAGGGGTAACCTGGGGCTGGAG | |
| | GGAAGCTGTACTGTCCCACCTAACGTTGCAACTGGGAGACTTCCGGGGGCCTCACTGC | |
| | TCCTAGAGCCCTCTTCGCTGACTGCCAATATGAAGGAGGTACCTCTGTTCAGGCTACG | |
| | TCACTTCCCTTGTGGGAATGTCAATTACGGCTACCAGCAACAGGGCTTGCCCTTAGAA | |
| | GCCGCTACTGCCCCTGGAGCTGGTCATTACGAGGATACCATTCTGAAAAGCAAGAATA | |
| | GCATGAACCAGCCTGGGCCCTGA | |
| ALK- | ATGGCGGTGGCCGAGCTGTACACGCAGTACAACAGGGTCTGGATTCCCGATCCTGAAG | 19 |
| MYO5C | AAGTTTGGAAGTCTGCTGAAATAGCCAAGGACTACAGAGTTGGTGACAAGGTCCTGCG | |
| | ACTCCTGCTGGAGGATGGAACGGAGCTGGATTATTCTGTCAATCCAGAATCTCTGCCT | |
| | CCACTTCGGAATCCTGACATCCTCGTGGGCGAGAATGACCTCACGGCTCTCAGCTATC | |
| | TTCACGAGCCCGCGGTGCTCCACAACCTCAGAATCCGCTTTGCAGAATCCAAACTCAT | |
| | TTACACCTACAGTGGAATCATTTTGGTGGCCATGAATCCTTACAAGCAGTTGCCAATA | |
| | TACGGAGATGCCATCATCCACGCCTACAGCGGGCAGAACATGGGCGATATGGACCCAC | |
| | ACATATTTGCCGTGGCAGAAGAGGCATACAAGCAGATGGCCAGAAACAACAGAAACCA | |
| | GTCCATAATTGTAAGTGGGGAGTCAGGTGCTGGAAAGACAGTGTCGGCTCGCTATGCC | |
| | ATGAGGTACTTTGCCACCGTCAGCAAATCGGGCAGCAACGCTCACGTGGAAGACAAGG | |
| | TCCTGGCATCCAATCCCATCACCGAGGCCGTTGGAAATGCCAAGACCACCCGCAATGA | |
| | CAATAGTAGTCGGTTTGGGAAATACACAGAAATCAGTTTTGATGAACAAAATCAAATT | |
| | ATAGGAGCCAACATGAGCACTTACCTCCTGGAGAAATCCAGAGTTGTCTTTCAATCGG | |
| | AAAATGAACGAAATTACCACATTTTCTATCAGCTTTGTGCATCTGCACAGCAGTCGGA | |
| | ATTTAAACATCTTAAATTGGGGAGTGCCGAAGAATTTAATTATACAAGAATGGGAGGC | |
| | AATACTGTCATTGAGGGTGTGAATGATCGAGCTGAAATGGTAGAGACTCAAAAGACCT | |
| | TCACGCTTCTGGGTTTCAAGGAGGATTTTCAGATGGACGTTTTTAAAATCCTGGCAGC | |
| | CATCCTACATCTGGGCAATGTGCAGATCACCGCGGTGGGCAACGAGAGGTCCTCAGTT | |
| | AGTGAGGATGACAGTCACCTGAAGGTGTTCTGTGAGCTCCTGGGCCTGGAGAGTGGCA | |
| | GAGTTGCTCAGTGGCTGTGCAATCGCAAAATCGTCACAAGCTCTGAGACGGTGGTAAA | |
| | ACCCATGACCAGGCCTCAGGCTGTCAACGCCAGGGATGCACTGGCCAAAAAGATCTAT | |
| | GCTCACCTGTTCGACTTCATTGTGGAGAGAATTAACCAAGCGTTGCAGTTTTCAGGCA | |
| | AGCAGCACACTTTTATTGGTGTTTTGGACATTTATGGTTTTGAAACCTTTGATGTGAA | |
| | CAGCTTTGAACAATTTTGCATCAATTACGCTAATGAAAAACTGCAACAACAGTTTAAC | |
| | ATGCATGTCTTCAAACTGGAACAAGAAGAATACATGAAGGAAGATATACCTTGGACGC | |
| | TGATAGATTTTTATGACAATCAACCAGTTATTGACCTGATTGAAGCAAAAATGGGAAT | |
| | TCTGGAGTTACTGGATGAAGAATGTTTGTTACCACATGGAACTGATGAAAACTGGCTT | |
| | CAAAAGCTGTATAATAATTTTGTCAACAGGAACCCTTTGTTTGAAAAGCCTAGAATGT | |
| | CAAACACATCCTTTGTCATCCAGCACTTTGCTGATAAGGTAGAGTATAAATGTGAAGG | |
| | TTTCCTGGAGAAAAACAGAGACACCGTCTATGACATGCTGGTTGAAATCCTGAGAGCA | |
| | AGCAAGTTTCATCTCTGTGCCAACTTTTTTCAAGAAAATCCAACTCCTCCTTCTCCTT | |
| | TTGGTTCAATGATTACAGTTAAATCTGCAAAGCAAGTCATCAAGCCAAACAGCAAGCA | |
| | TTTCCGGACCACAGTTGGGAGCAAGTTCCGCAGCTCTCTGTACTTGCTCATGGAGACC | |
| | CTCAATGCGACGACGCCCCACTACGTTCGATGCATCAAGCCAAATGATGAGAAGTTAC | |
| | CCTTTGAATTTGACTCCAAAAGAATTGTTCAGCAGCTGCGAGCCTGCGGCGTTTTAGA | |
| | AACGATTCGCATTAGTGCACAGAGCTACCCTTCCAGGTGGACATACATCGAGTTCTAC | |
| | AGTCGCTACGGCATTCTCATGACCAAGCAAGAGCTTTCCTTCAGCGATAAAAAGGAGG | |
| | TGTGCAAGGTGGTTTTACACAGACTCATCCAGGATTCTAATCAGTACCAGTTTGGTAA | |
| | AACCAAAATTTTCTTCAGAGCAGGACAAGTGGCTTATTTAGAGAAACTTCGATTGGAT | |
| | AAACTGAGGCAGAGTTGTGTTATGGTACAAAAGCACATGCGTGGCTGGCTCCAGAGGA | |
| | AAAAATTCCTCCGAGAGAGACGAGCCGCCCTGATAATCCAGCAGTACTTCCGGGGTCA | |
| | GCAAACTGTGAGGAAAGCTATTACTGCAGTGGCCTTAAAAGAAGCTTGGGCAGCCATA | |
| | ATCATTCAGAAGCACTGCCGCGGGTATCTTGTTCGCAGCCTGTATCAGTTGATTCGCA | |
| | TGGCCACCATCACAATGCAGGCCTACAGCCGAGGATTCCTGGCAAGGAGGAGGTATCG | |
| | AAAGATGCTGGAGGAACATAAGGCTGTGATCCTACAGAAATACGCACGGGCGTGGCTG | |
| | GCCAGACGCAGATTCCAGAGTATCCGACGATTCGTGCTTAATATTCAGCTTACTTACA | |
| | GGGTCCAGCGTTTGCAGAAAAAGTTGGAAGATCAGAACAAAGAAAACCATGGGCTGGT | |
| | GGAGAAGCTGACTAGCCTGGCTGCTCTTCGAGCTGGGGATGTGGAAAAGATTCAGAAG | |
| | CTGGAAGCAGAACTAGAAAAAGCAGCCACTCACAGGCGAAATTACGAGGAGAAGGGGA | |
| | AGAGATACAGGGATGCTGTGGAAGAGAAATTGGCAAAGCTTCAGAAGCATAATTCAGA | |
| | ACTGGAAACACAGAAAGAACAAATACAGCTGAAGCTTCAAGAGAAGACTGAAGAGTTA | |
| | AAAGAAAAAATGGACAACCTCACCAAGCAGCTCTTTGATGATGTACAAAAGGAAGAAC | |
| | GGCAAAGAATGCTTCTTGAAAAAAGTTTTGAACTGAAAACACAAGACTATGAGAAGCA | |
| | GATTCAGTCTTTGAAAGAAGAAATTAAAGCTCTCAAGGATGAGAAGATGCAACTCCAA | |
| | CACCTGGTGGAGGGGGAGCACGTCACTTCTGATGGCTTGAAGGCGGAAGTGGCCCGCC | |
| | TGAGCAAGCAGGTCAAGACAATCTCTGAGTTCGAAAAAGAGATAGAACTACTTCAGGC | |
| | ACAGAAGATAGATGTGGAGAAACATGTGCAGTCACAAAAACGGGAAATGAGAGAAAAG | |
| | ATGTCAGAGATCACCAAACAACTTCTCGAAAGCTATGACATTGAAGATGTAAGAAGCA | |
| | GGCTCTCTGTGGAAGATCTGGAACATTTAAATGAGGATGGAGAACTTTGGTTTGCTTA | |
| | TGAAGGACTAAAGAAAGCAACACGTGTTTTGGAGAGCCATTTCCAGTCTCAGAAGGAT | |
| | TGCTATGAAAAGGAGATTGAAGCTTTGAACTTCAAAGTGGTGCATCTCAGTCAAGAAA | |
| | TCAACCACCTGCAGAAGTTATTCAGAGAAGAAAATGACATCAATGAAAGCATCCGTCA | |
| | TGAAGTTACCAGGCTAACATCAGAGAACATGATGATCCCAGACTTTAAACAGCAAATT | |
| | TCAGAATTGGAGAAACAGAAGCAAGATCTTGAAATCCGCCTGAATGAACAAGCTGAGA | |
| | AAATGAAAGTGTACCGCCGGAAGCACCAGGAGCTGCAAGCCATGCAGATGGAGCTGCA | |
| | GAGCCCTGAGTACAAGCTGAGCAAGCTCCGCACCTCGACCATCATGACCGACTACAAC | |
| | CCCAACTACTGCTTTGCTGGCAAGACCTCCTCCATCAGTGACCTGAAGGAGGTGCCGC | |
| | GGAAAAACATCACCCTCATTCGGGGTCTGGGCCATGGCGCCTTTGGGGAGGTGTATGA | |
| | AGGCCAGGTGTCCGGAATGCCCAACGACCCAAGCCCCCTGCAAGTGGCTGTGAAGACG | |
| | CTGCCTGAAGTGTGCTCTGAACAGGACGAACTGGATTTCCTCATGGAAGCCCTGATCA | |
| | TCAGCAAATTCAACCACCAGAACATTGTTCGCTGCATTGGGGTGAGCCTGCAATCCCT | |
| | GCCCCGGTTCATCCTGCTGGAGCTCATGGCGGGGGGAGACCTCAAGTCCTTCCTCCGA | |
| | GAGACCCGCCCTCGCCCGAGCCAGCCCTCCTCCCTGGCCATGCTGGACCTTCTGCACG | |
| | TGGCTCGGGACATTGCCTGTGGCTGTCAGTATTTGGAGGAAAACCACTTCATCCACCG | |
| | AGACATTGCTGCCAGAAACTGCCTCTTGACCTGTCCAGGCCCTGGAAGAGTGGCCAAG | |
| | ATTGGAGACTTCGGGATGGCCCGAGACATCTACAGGGCGAGCTACTATAGAAAGGGAG | |
| | GCTGTGCCATGCTGCCAGTTAAGTGGATGCCCCCAGAGGCCTTCATGGAAGGAATATT | |
| | CACTTCTAAAACAGACACATGGTCCTTTGGAGTGCTGCTATGGGAAATCTTTTCTCTT | |
| | GGATATATGCCATACCCCAGCAAAAGCAACCAGGAAGTTCTGGAGTTTGTCACCAGTG | |
| | GAGGCCGGATGGACCCACCCAAGAACTGCCCTGGGCCTGTATACCGGATAATGACTCA | |
| | GTGCTGGCAACATCAGCCTGAAGACAGGCCCAACTTTGCCATCATTTTGGAGAGGATT | |
| | GAATACTGCACCCAGGACCCGGATGTAATCAACACCGCTTTGCCGATAGAATATGGTC | |
| | CACTTGTGGAAGAGGAAGAGAAAGTGCCTGTGAGGCCCAAGGACCCTGAGGGGGTTCC | |
| | TCCTCTCCTGGTCTCTCAACAGGCAAAACGGGAGGAGGAGCGCAGCCCAGCTGCCCCA | |
| | CCACCTCTGCCTACCACCTCCTCTGGCAAGGCTGCAAAGAAACCCACAGCTGCAGAGA | |
| | TCTCTGTTCGAGTCCCTAGAGGGCCGGCCGTGGAAGGGGGACACGTGAATATGGCATT | |
| | CTCTCAGTCCAACCCTCCTTCGGAGTTGCACAAGGTCCACGGATCCAGAAACAAGCCC | |
| | ACCAGCTTGTGGAACCCAACGTACGGCTCCTGGTTTACAGAGAAACCCACCAAAAAGA | |
| | ATAATCCTATAGCAAAGAAGGAGCCACACGACAGGGGTAACCTGGGGCTGGAGGGAAG | |
| | CTGTACTGTCCCACCTAACGTTGCAACTGGGAGACTTCCGGGGGCCTCACTGCTCCTA | |
| | GAGCCCTCTTCGCTGACTGCCAATATGAAGGAGGTACCTCTGTTCAGGCTACGTCACT | |
| | TCCCTTGTGGGAATGTCAATTACGGCTACCAGCAACAGGGCTTGCCCTTAGAAGCCGC | |
| | TACTGCCCCTGGAGCTGGTCATTACGAGGATACCATTCTGAAAAGCAAGAATAGCATG | |
| | AACCAGCCTGGGCCCTGA | |
| ALK- | ATGAACTACCAGCAGCAGCTGGCCAACTCGGCTGCCATCCGGGCCGAGATCCAGCGCT | 20 |
| AGAP1 | TCGAGTCGGTCCACCCCAACATCTACTCCATCTACGAGCTGCTGGAGCGCGTGGAGGA | |
| | GCCGGTGCTGCAGAACCAGATCCGGGAGCACGTCATCGCCATCGAAGATGCCTTCGTG | |
| | AACAGCCAGGAATGGACGCTGAGTCGATCTGTCCCGGAGCTCAAAGTGGGAATTGTGG | |
| | GTAACTTGGCCAGCGGCAAGTCTGCCCTGGTGCACCGGTACCTGACGGGCACATATGT | |
| | CCAGGAGGAGTCTCCGGAAGGTGGCAGGTTCAAGAAAGAGATTGTCGTTGATGGACAG | |
| | AGCTATCTGCTGCTGATCAGAGATGAAGGGGGCCCCCCGGAGGCGCAGTTTGCCATGT | |
| | GGGTGGACGCTGTTATATTTGTCTTCAGCTTGGAGGATGAAATAAGTTTCCAGACCGT | |
| | TTACCACTACTACAGTCGAATGGCCAACTATCGGAACACGAGCGAGATTCCTCTGGTT | |
| | CTGGTGGGAACCCAGGATGCCATAAGTTCTGCTAACCCGAGGGTCATCGATGACGCCA | |
| | GGGCGAGGAAGCTCTCCAACGACCTGAAACGGTGCACGTACTACGAGACGTGTGCTAC | |
| | ATACGGGCTGAATGTGGAGAGGGTCTTCCAGGACGTGATGGAAGGCCACGGGGAAGTG | |
| | AATATTAAGCATTATCTAAACTGCAGTCACTGTGAGGTAGACGAATGTCACATGGACC | |
| | CTGAAAGCCACAAGGTCATCTGCTTCTGTGACCACGGGACGGTGCTGGCTGAGGATGG | |
| | CGTCTCCTGCATTGTGTCACCCACCCCGGAGCCACACCTGCCACTCTCGCTGATCCTC | |
| | TCTGTGGTGACCTCTGCCCTCGTGGCCGCCCTGGTCCTGGCTTTCTCCGGCATCATGA | |
| | TTGTGTACCGCCGGAAGCACCAGGAGCTGCAAGCCATGCAGATGGAGCTGCAGAGCCC | |
| | TGAGTACAAGCTGAGCAAGCTCCGCACCTCGACCATCATGACCGACTACAACCCCAAC | |
| | TACTGCTTTGCTGGCAAGACCTCCTCCATCAGTGACCTGAAGGAGGTGCCGCGGAAAA | |
| | ACATCACCCTCATTCGGGGTCTGGGCCATGGCGCCTTTGGGGAGGTGTATGAAGGCCA | |
| | GGTGTCCGGAATGCCCAACGACCCAAGCCCCCTGCAAGTGGCTGTGAAGACGCTGCCT | |
| | GAAGTGTGCTCTGAACAGGACGAACTGGATTTCCTCATGGAAGCCCTGATCATCAGCA | |
| | AATTCAACCACCAGAACATTGTTCGCTGCATTGGGGTGAGCCTGCAATCCCTGCCCCG | |
| | GTTCATCCTGCTGGAGCTCATGGCGGGGGGAGACCTCAAGTCCTTCCTCCGAGAGACC | |
| | CGCCCTCGCCCGAGCCAGCCCTCCTCCCTGGCCATGCTGGACCTTCTGCACGTGGCTC | |
| | GGGACATTGCCTGTGGCTGTCAGTATTTGGAGGAAAACCACTTCATCCACCGAGACAT | |
| | TGCTGCCAGAAACTGCCTCTTGACCTGTCCAGGCCCTGGAAGAGTGGCCAAGATTGGA | |
| | GACTTCGGGATGGCCCGAGACATCTACAGGGCGAGCTACTATAGAAAGGGAGGCTGTG | |
| | CCATGCTGCCAGTTAAGTGGATGCCCCCAGAGGCCTTCATGGAAGGAATATTCACTTC | |
| | TAAAACAGACACATGGTCCTTTGGAGTGCTGCTATGGGAAATCTTTTCTCTTGGATAT | |
| | ATGCCATACCCCAGCAAAAGCAACCAGGAAGTTCTGGAGTTTGTCACCAGTGGAGGCC | |
| | GGATGGACCCACCCAAGAACTGCCCTGGGCCTGTATACCGGATAATGACTCAGTGCTG | |
| | GCAACATCAGCCTGAAGACAGGCCCAACTTTGCCATCATTTTGGAGAGGATTGAATAC | |
| | TGCACCCAGGACCCGGATGTAATCAACACCGCTTTGCCGATAGAATATGGTCCACTTG | |
| | TGGAAGAGGAAGAGAAAGTGCCTGTGAGGCCCAAGGACCCTGAGGGGGTTCCTCCTCT | |
| | CCTGGTCTCTCAACAGGCAAAACGGGAGGAGGAGCGCAGCCCAGCTGCCCCACCACCT | |
| | CTGCCTACCACCTCCTCTGGCAAGGCTGCAAAGAAACCCACAGCTGCAGAGATCTCTG | |
| | TTCGAGTCCCTAGAGGGCCGGCCGTGGAAGGGGGACACGTGAATATGGCATTCTCTCA | |
| | GTCCAACCCTCCTTCGGAGTTGCACAAGGTCCACGGATCCAGAAACAAGCCCACCAGC | |
| | TTGTGGAACCCAACGTACGGCTCCTGGTTTACAGAGAAACCCACCAAAAAGAATAATC | |
| | CTATAGCAAAGAAGGAGCCACACGACAGGGGTAACCTGGGGCTGGAGGGAAGCTGTAC | |
| | TGTCCCACCTAACGTTGCAACTGGGAGACTTCCGGGGGCCTCACTGCTCCTAGAGCCC | |
| | TCTTCGCTGACTGCCAATATGAAGGAGGTACCTCTGTTCAGGCTACGTCACTTCCCTT | |
| | GTGGGAATGTCAATTACGGCTACCAGCAACAGGGCTTGCCCTTAGAAGCCGCTACTGC | |
| | CCCTGGAGCTGGTCATTACGAGGATACCATTCTGAAAAGCAAGAATAGCATGAACCAG | |
| | CCTGGGCCCTGA | |
| ALK- | ATGGGAGCCATCGGGCTCCTGTGGCTCCTGCCGCTGCTGCTTTCCACGGCAGCTGTGG | 21 |
| MED13L | GCTCCGGGATGGGGACCGGCCAGCGCGCGGGCTCCCCAGCTGCGGGGCCGCCGCTGCA | |
| | GCCCCGGGAGCCACTCAGCTACTCGCGCCTGCAGAGGAAGAGTCTGGCAGTTGACTTC | |
| | GTGGTGCCCTCGCTCTTCCGTGTCTACGCCCGGGACCTACTGCTGCCACCATCCTCCT | |
| | CGGAGCTGAAGGCTGGCAGGCCCGAGGCCCGCGGCTCGCTAGCTCTGGACTGCGCCCC | |
| | GCTGCTCAGGTTGCTGGGGCCGGCGCCGGGGGTCTCCTGGACCGCCGGTTCACCAGCC | |
| | CCGGCAGAGGCCCGGACGCTGTCCAGGGTGCTGAAGGGCGGCTCCGTGCGCAAGCTCC | |
| | GGCGTGCCAAGCAGTTGGTGCTGGAGCTGGGCGAGGAGGCGATCTTGGAGGGTTGCGT | |
| | CGGGCCCCCCGGGGAGGCGGCTGTGGGGCTGCTCCAGTTCAATCTCAGCGAGCTGTTC | |
| | AGTTGGTGGATTCGCCAAGGCGAAGGGCGACTGAGGATCCGCCTGATGCCCGAGAAGA | |
| | AGGCGTCGGAAGTGGGCAGAGAGGGAAGGCTGTCCGCGGCAATTCGCGCCTCCCAGCC | |
| | CCGCCTTCTCTTCCAGATCTTCGGGACTGTACTGGTAAGTCCTTATGGCTTAAATGGG | |
| | ACGCTAACAGGCCAAGCATACAAGATGTCAGACCCAGCCACTCGTAAGTTGATTGAGG | |
| | AATGGCAGTATTTCTACCCGATGGTGCTAAAAAAGAAAGAAGAATCGAAAGAGGAAGA | |
| | CGAGTTGGGATATGATGATGATTTCCCTGTGGCAGTTGAAGTAATTGTTGGTGGTGTT | |
| | CGGATGGTTTACCCTTCAGCATTTGTTTTGATCTCTCAGAATGACATCCCGGTTCCTC | |
| | AGAGTGTTGCCAGTGCTGGAGGCCACATTGCAGTTGGGCAGCAAGGGCTTGGTAGTGT | |
| | GAAGGACCCAAGTAACTGTGGGATGCCTCTGACCCCTCCCACCTCTCCAGAACAGGCT | |
| | ATCCTAGGTGAGAGTGGAGGTATGCAGAGTGCTGCCAGTCACCTGGTTTCCCAAGATG | |
| | GAGGGATGATAACGATGCACAGTCCAAAGAGATCGGGGAAGATTCCTCCAAAACTCCA | |
| | CAATCATATGGTCCATCGAGTCTGGAAGGAATGCATCCTCAACAGAACCCAGTCCAAG | |
| | AGGAGCCAAATGTCAACTCCAACTCTTGAAGAAGAGCCTGCTAGCAATCCTGCTACTT | |
| | GGGATTTTGTGGATCCAACCCAAAGAGTCAGCTGTTCTTGTTCCAGGCATAAGCTTTT | |
| | AAAACGTTGTGCAGTCGGGCCCAATCGACCTCCCACAGTATCTCAACCAGGGTTCAGT | |
| | GCAGGACCATCATCATCTTCATCTTTACCACCTCCTGCTTCTTCTAAGCACAAAACAG | |
| | CAGAAAGACAGGAAAAAGGAGACAAGCTGCAAAAGAGACCCTTAATACCATTTCACCA | |
| | TAGGCCCTCTGTGGCCGAAGAATTATGCATGGAGCAAGATACACCAGGACAGAAACTA | |
| | GGGTTGGCAGGGATAGACTCCTCCTTAGAGGTGTCTAGCAGTAGGAAATATGATAAGC | |
| | AAATGGCCGTGCCTTCCAGAAATACAAGCAAGCAAATGAATCTGAATCCTATGGATTC | |
| | ACCTCATTCCCCTATATCCCCTCTGCCACCAACACTCAGCCCTCAGCCACGAGGTCAG | |
| | GAAACAGAGAGTTTGGACCCACCATCGGTCCCTGTGAATCCAGCCCTTTATGGAAATG | |
| | GACTAGAACTCCAGCAGTTGTCTACTCTGGATGACAGAACTGTCCTCGTAGGCCAAAG | |
| | ACTGCCTCTCATGGCAGAGGTCAGCGAGACAGCCTTATATTGTGGGATTAGGCCCTCG | |
| | AACCCGGAGTCATCAGAAAAGTGGTGGCATAGTTATCGTCTCCCACCCAGTGATGATG | |
| | CTGAGTTCAGGCCTCCAGAGCTCCAGGGTGAGAGATGTGATGCCAAAATGGAGGTAAA | |
| | CTCAGAGAGCACTGCATTGCAAAGACTCTTAGCACAACCTAACAAACGGTTTAAAATC | |
| | TGGCAAGACAAACAGCCCCAGTTGCAGCCACTCCACTTCCTTGACCCATTGCCTCTAT | |
| | CACAACAACCTGGAGACAGTTTGGGAGAAGTGAATGACCCATATACCTTTGAAGATGG | |
| | TGACATAAAATACATCTTTACAGCCAACAAGAAATGCAAACAAGGGACGGAGAAAGAT | |
| | TCCCTGAAAAAGAATAAGTCAGAGGATGGATTTGGTACCAAGGATGTCACTACACCAG | |
| | GTCATTCCACGCCGGTGCCTGATGGGAAAAATGCCATGTCTATTTTCAGTTCTGCTAC | |
| | TAAAACAGATGTCCGGCAGGATAATGCTGCTGGCAGAGCTGGCTCCAGTAGCCTTACA | |
| | CAGGTAACAGATTTGGCACCTTCCCTGCATGACTTAGACAACATCTTTGATAATTCTG | |
| | ATGACGACGAACTTGGGGCTGTATCACCTGCTCTGCGCTCATCAAAAATGCCTGCAGT | |
| | TGGGACAGAAGACCGACCTCTTGGGAAGGATGGAAGAGCTGCTGTTCCTTATCCACCA | |
| | ACAGTTGCAGACTTGCAAAGGATGTTTCCCACTCCACCATCTTTGGAACAGCATCCTG | |
| | CATTTTCTCCTGTGATGAATTATAAAGATGGGATCAGCTCAGAGACAGTGACAGCATT | |
| | AGGCATGATGGAGAGCCCTATGGTCAGTATGGTTTCAACACAACTCACAGAATTCAAA | |
| | ATGGAAGTGGAAGATGGATTAGGAAGTCCCAAGCCCGAGGAAATTAAGGACTTTTCAT | |
| | ATGTGCACAAAGTTCCATCCTTTCAACCTTTTGTGGGATCCTCCATGTTTGCTCCACT | |
| | GAAGATGTTGCCGAGCCATTGTTTGCTACCTCTGAAGATACCTGATGCCTGTCTGTTT | |
| | CGGCCTTCATGGGCAATTCCTCCTAAAATTGAACAACTGCCCATGCCCCCTGCAGCCA | |
| | CTTTCATTAGAGATGGCTACAATAACGTGCCTAGTGTTGGGAGCCTAGCAGATCCAGA | |
| | CTATCTGAACACACCACAGATGAACACACCCGTGACGTTGAACAGCGCTGCCCCAGCC | |
| | AGCAATAGTGGGGCAGGAGTCCTACCATCTCCAGCAACCCCTCGCTTCTCTGTCCCCA | |
| | CACCACGAACCCCCAGGACCCCAAGAACTCCCAGAGGTGGGGGCACTGCCAGTGGTCA | |
| | AGGGTCTGTTAAGTATGATAGCACCGATCAAGGATCACCAGCCTCCACCCCCTCTACT | |
| | ACACGGCCCCTCAACTCTGTGGAGCCCGCCACCATGCAGCCAATTCCCGAAGCCCACA | |
| | GCCTCTATGTTACCCTGATTCTCTCCGATTCCGTGATGAATATCTTTAAAGACAGAAA | |
| | CTTTGACAGCTGTTGCATCTGTGCCTGCAACATGAACATCAAAGGGGCGGATGTCGGG | |
| | CTTTACATCCCCGATTCTTCCAATGAGGACCAGTACCGCTGTACCTGTGGGTTTAGTG | |
| | CGATTATGAACCGCAAACTTGGCTACAATTCAGGACTCTTCCTTGAAGATGAGTTGGA | |
| | TATTTTTGGGAAGAATTCTGATATTGGTCAGGCTGCAGAGAGGCGCTTAATGATGTGT | |
| | CAGTCCACCTTCCTTCCTCAGGTGGAAGGAACCAAAAAACCCCAGGAGCCACCCATAA | |
| | GCCTTCTCCTCCTCCTCCAGAATCAACACACACAACCTTTTGCTTCACTGAATTTCCT | |
| | GGACTACATTTCCTCTAACAATCGCCAAACTCTTCCCTGTGTAAGCTGGAGTTATGAC | |
| | CGGGTGCAAGCAGATAATAATGATTACTGGACGGAATGCTTTAATGCGTTGGAGCAGG | |
| | GGCGGCAGTATGTGGATAACCCCACTGGTGGAAAAGTGGACGAAGCTCTGGTGAGAAG | |
| | TGCCACTGTGCACTCTTGGCCTCACAGCAATGTGCTGGACATCAGCATGCTCTCCTCC | |
| | CAGGATGTGGTTCGTATGCTGTTGTCCCTGCAGCCCTTTCTCCAAGATGCCATCCAAA | |
| | AGAAGCGCACGGGCAGGACCTGGGAGAACATCCAGCATGTGCAGGGACCACTCACTTG | |
| | GCAGCAGTTCCATAAAATGGCAGGACGGGGAACCTACGGTTCGGAAGAATCTCCTGAG | |
| | CCGTTGCCCATCCCCACTCTGCTGGTAGGCTATGACAAGGATTTCCTCACCATCTCGC | |
| | CATTCTCCTTGCCGTTTTGGGAGAGGCTCTTGTTGGACCCATATGGGGGCCACCGTGA | |
| | TGTTGCCTATATTGTGGTGTGTCCAGAAAATGAGGCCTTGCTCGAAGGAGCCAAAACT | |
| | TTCTTCAGGGACTTGAGTGCTGTATACGAGATGTGTAGGCTTGGGCAGCACAAGCCCA | |
| | TCTGCAAAGTGCTACGTGACGGGATCATGCGCGTGGGAAAAACTGTGGCACAGAAGCT | |
| | GACAGATGAGCTTGTGAGTGAGTGGTTTAACCAGCCTTGGAGCGGCGAGGAGAATGAC | |
| | AATCATTCCAGACTCAAACTTTATGCGCAAGTTTGCCGCCATCACCTAGCACCTTATT | |
| | TAGCCACTCTGCAGCTTGATAGCAGCCTATTGATACCACCTAAATACCAGACCCCACC | |
| | AGCAGCAGCACAGGGACAAGCTACGCCAGGGAATGCTGGGCCCTTAGCTCCAAATGGA | |
| | TCAGCAGCTCCCCCAGCTGGCAGTGCATTTAATCCCACCTCGAATAGTAGTTCTACAA | |
| | ATCCTGCAGCAAGTAGTTCTGCATCTGGTTCCTCTGTGCCACCGGTCTCATCGTCTGC | |
| | CTCTGCTCCTGGTATTAGCCAGATAAGCACTACCTCTTCTTCAGGATTCAGTGGTAGT | |
| | GTTGGAGGGCAGAACCCCAGCACTGGGGGCATTTCTGCGGATAGAACGCAAGGGAACA | |
| | TAGGCTGTGGTGGAGACACTGACCCTGGGCAGAGCTCTTCTCAGCCCTCACAGGATGG | |
| | ACAAGAGAGTGTTACAGAAAGGGAGAGAATAGGAATTCCCACGGAGCCTGACTCTGCA | |
| | GACAGCCATGCCCACCCTCCAGCTGTTGTCATTTACATGGTGGACCCGTTCACGTATG | |
| | CTGCAGAGGAGGACTCCACTTCTGGGAACTTTTGGCTGTTGAGCTTGATGCGCTGCTA | |
| | CACAGAAATGCTGGATAATTTACCTGAGCATATGAGAAATTCTTTCATTCTCCAGATT | |
| | GTGCCTTGCCAGTACATGCTGCAGACAATGAAGGATGAGCAAGTTTTCTACATTCAAT | |
| | ACTTGAAGTCCATGGCATTTTCAGTGTACTGCCAGTGCAGGCGACCACTGCCTACACA | |
| | GATCCACATTAAATCCCTCACGGGATTTGGGCCTGCAGCCAGCATTGAGATGACCCTC | |
| | AAGAACCCTGAGCGGCCCAGCCCAATCCAGCTTTACTCCCCTCCCTTTATATTGGCCC | |
| | CAATCAAAGACAAGCAGACAGAGCTGGGAGAGACGTTTGGTGAGGCGAGCCAGAAATA | |
| | CAATGTGCTCTTCGTGGGCTATTGTCTGTCTCACGACCAGCGCTGGCTTTTGGCTTCC | |
| | TGCACTGACCTCCATGGGGAATTATTAGAGACCTGCGTTGTAAATATTGCTTTACCAA | |
| | ACAGGTCACGGAGGAGTAAAGTATCTGCACGTAAAATTGGACTACAGAAGTTATGGGA | |
| | GTGGTGCATAGGGATTGTCCAAATGACATCTCTACCCTGGAGAGTTGTAATCGGGCGA | |
| | CTTGGGCGTCTTGGCCATGGGGAGCTTAAAGATTGGAGTATCCTCCTTGGAGAATGTT | |
| | CACTACAGACAATCAGCAAAAAGCTCAAGGATGTGTGCCGGATGTGTGGAATCTCTGC | |
| | CGCAGACTCTCCTTCTATCCTTAGTGCCTGCCTGGTTGCCATGGAGCCCCAGGGGTCC | |
| | TTTGTAGTGATGCCAGATGCTGTCACAATGGGCTCTGTTTTTGGCCGAAGTACTGCAC | |
| | TGAACATGCAGTCATCTCAGCTCAACACCCCTCAAGATGCTTCTTGTACACACATCTT | |
| | GGTGTTCCCAACATCATCAACCATCCAGGTGGCTCCAGCCAACTACCCCAATGAAGAT | |
| | GGGTTTAGCCCCAACAATGATGATATGTTTGTTGACCTTCCATTCCCAGATGATATGG | |
| | ACAATGATATTGGCATATTAATGACTGGGAACCTCCATTCCTCTCCCAACTCTTCCCC | |
| | AGTACCCTCCCCAGGCTCTCCTTCTGGAATTGGTGTGGGCTCTCACTTCCAGCATAGT | |
| | CGGAGCCAGGGTGAGCGTCTTCTTTCTAGAGAAGCACCAGAGGAGCTAAAGCAGCAGC | |
| | CCCTGGCCCTTGGGTATTTTGTATCAACTGCCAAAGCTGAGAATCTTCCCCAGTGGTT | |
| | TTGGTCATCGTGTCCCCAGGCTCAAAACCAGTGCCCTCTCTTCTTAAAGGCTTCGCTG | |
| | CATCACCACATTTCAGTAGCACAGACAGACGAACTTCTGCCTGCCAGGAATTCTCAGC | |
| | GGGTTCCACACCCTCTTGACTCCAAAACCACGTCGGATGTTTTAAGGTTTGTTTTGGA | |
| | GCAGTACAACGCTCTGTCCTGGCTCACGTGCAATCCGGCCACCCAGGACCGTACTTCC | |
| | TGCCTTCCCGTCCACTTTGTGGTGCTCACTCAGTTGTACAATGCCATCATGAATATAC | |
| | TTTAA | |
| ALK-MTBP | ATGGGAGCCATCGGGCTCCTGTGGCTCCTGCCGCTGCTGCTTTCCACGGCAGCTGTGG | 22 |
| | GCTCCGGGATGGGGACCGGCCAGCGCGCGGGCTCCCCAGCTGCGGGGCCGCCGCTGCA | |
| | GCCCCGGGAGCCACTCAGCTACTCGCGCCTGCAGAGGAAGAGTCTGGCAGTTGACTTC | |
| | GTGGTGCCCTCGCTCTTCCGTGTCTACGCCCGGGACCTACTGCTGCCACCATCCTCCT | |
| | CGGAGCTGAAGGCTGGCAGGCCCGAGGCCCGCGGCTCGCTAGCTCTGGACTGCGCCCC | |
| | GCTGCTCAGGTTGCTGGGGCCGGCGCCGGGGGTCTCCTGGACCGCCGGTTCACCAGCC | |
| | CCGGCAGAGGCCCGGACGCTGTCCAGGGTGCTGAAGGGCGGCTCCGTGCGCAAGCTCC | |
| | GGCGTGCCAAGCAGTTGGTGCTGGAGCTGGGCGAGGAGGCGATCTTGGAGGGTTGCGT | |
| | CGGGCCCCCCGGGGAGGCGGCTGTGGGGCTGCTCCAGTTCAATCTCAGCGAGCTGTTC | |
| | AGTTGGTGGATTCGCCAAGGCGAAGGGCGACTGAGGATCCGCCTGATGCCCGAGAAGA | |
| | AGGCGTCGGAAGTGGGCAGAGAGGGAAGGCTGTCCGCGGCAATTCGCGCCTCCCAGCC | |
| | CCGCCTTCTCTTCCAGATCTTCGGGACTGGTCATAGCTCCTTGGAATCACCAACAAAC | |
| | ATGCCTTCTCCTTCTCCTGATTATTTTACATGGAATCTCACCTGGATAATGAAAGACT | |
| | CCTTCCCTTTCCTGTCTCATCGCAGCCGATATGGTCTGGAGTGCAGCTTTGACTTCCC | |
| | CTGTGAGCTGGAGTATTCCCCTCCACTGCATGACCTCAGGAACCAGAGCTGGTCCTGG | |
| | CGCCGCATCCCCTCCGAGGAGGCCTCCCAGATGGACTTGCTGGATGGGCCTGGGGCAG | |
| | AGCGTTCTAAGGAGATGCCCAGAGGCTCCTTTCTCCTTCTCAACACCTCAGCTGACTC | |
| | CAAGCACACCATCCTGAGTCCGTGGATGAGGAGCAGCAGTGAGCACTGCACACTGGCC | |
| | GTCTCGGTGCACAGGCACCTGCAGCCCTCTGGAAGGTACATTGCCCAGCTGCTGCCCC | |
| | ACAACGAGGCTGCAAGAGAGATCCTCCTGATGCCCACTCCAGGGAAGCATGGTTGGAC | |
| | AGTGCTCCAGGGAAGAATCGGGCGTCCAGACAACCCATTTCGAGTGGCCCTGGAATAC | |
| | ATCTCCAGTGGAAACCGCAGCTTGTCTGCAGTGGACTTCTTTGCCCTGAAGAACTGCA | |
| | GTGAAGGAACATCCCCAGGCTCCAAGATGGCCCTGCAGAGCTCCTTCACTTGTTGGAA | |
| | TGGGACAGTCCTCCAGCTTGGGCAGGCCTGTGACTTCCACCAGGACTGTGCCCAGGGA | |
| | GAAGATGAGAGCCAGATGTGCCGGAAACTGCCTGTGGGTTTTTACTGCAACTTTGAAG | |
| | ATGGCTTCTGTGGCTGGACCCAAGGCACACTGTCACCCCACACTCCTCAATGGCAGGT | |
| | CAGGACCCTAAAGGATGCCCGGTTCCAGGACCACCAAGACCATGCTCTATTGCTCAGT | |
| | ACCACTGATGTCCCCGCTTCTGAAAGTGCTACAGTGACCAGTGCTACGTTTCCTGCAC | |
| | CGATCAAGAGCTCTCCATGTGAGCTCCGAATGTCCTGGCTCATTCGTGGAGTCTTGAG | |
| | GGGAAACGTGTCCTTGGTGCTAGTGGAGAACAAAACCGGGAAGGAGCAAGGCAGGATG | |
| | GTCTGGCATGTCGCCGCCTATGAAGGCTTGAGCCTGTGGCAGTGGATGGTGTTGCCTC | |
| | TCCTCGATGTGTCTGACAGGTTCTGGCTGCAGATGGTCGCATGGTGGGGACAAGGATC | |
| | CAGAGCCATCGTGGCTTTTGACAATATCTCCATCAGCCTGGACTGCTACCTCACCATT | |
| | AGCGGAGAGGACAAGATCCTGCAGAATACAGCACCCAAATCAAGAAACCTGTTTGAGA | |
| | GAAACCCAAACAAGGAGCTGAAACCCGGGGAAAATTCACCAAGACAGACCCCCATCTT | |
| | TGACCCTACAGTTCATTGGCTGTTCACCACATGTGGGGCCAGCGGGCCCCATGGCCCC | |
| | ACCCAGGCACAGTGCAACAACGCCTACCAGAACTCCAACCTGAGCGTGGAGGTGGGGA | |
| | GCGAGGGCCCCCTGAAAGGCATCCAGATCTGGAAGGTGCCAGCCACCGACACCTACAG | |
| | CATCTCGGGCTACGGAGCTGCTGGCGGGAAAGGCGGGAAGAACACCATGATGCGGTCC | |
| | CACGGCGTGTCTGTGCTGGGCATCTTCAACCTGGAGAAGGATGACATGCTGTACATCC | |
| | TGGTTGGGCAGCAGGGAGAGGACGCCTGCCCCAGTACAAACCAGTTAATCCAGAAAGT | |
| | CTGCATTGGAGAGAACAATGTGATAGAAGAAGAAATCCGTGTGAACAGAAGCGTGCAT | |
| | GAGTGGGCAGGAGGCGGAGGAGGAGGGGGTGGAGCCACCTACGTATTTAAGATGAAGG | |
| | ATGGAGTGCCGGTGCCCCTGATCATTGCAGCCGGAGGTGGTGGCAGGGCCTACGGGGC | |
| | CAAGACAGACACGTTCCACCCAGAGAGACTGGAGAATAACTCCTCGGTTCTAGGGCTA | |
| | AACGGCAATTCCGGAGCCGCAGGTGGTGGAGGTGGCTGGAATGATAACACTTCCTTGC | |
| | TCTGGGCCGGAAAATCTTTGCAGGAGGGTGCCACCGGAGGACATTCCTGCCCCCAGGC | |
| | CATGAAGAAGTGGGGGTGGGAGACAAGAGGGGGTTTCGGAGGGGGTGGAGGGGGGTGC | |
| | TCCTCAGGTGGAGGAGGCGGAGGATATATAGGCGGCAATGCAGCCTCAAACAATGACC | |
| | CCGAAATGGATGGGGAAGATGGGGTTTCCTTCATCAGTCCACTGGGCATCCTGTACAC | |
| | CCCAGCTTTAAAAGTTCCAGATGTTGAAGTGAAAGGAGAGTGTTCTAGCTATTATCTC | |
| | TTGTTACAAGGTAATGGCAATAGAAGATGTAAAGCCACATTGATTCACTCAGCCAACC | |
| | AGATCAATGGCTCATTTGCACTCAATTTAATTCATGGAAAGATGAAAACAAAGACAGA | |
| | AGAAGCCAAATTGAGTTTTCCTTTTGACTTATTATCACTTCCACATTTTTCTGGGGAG | |
| | CAGATTGTACAGAGAGAGAAACAGTTAGCTAATGTTCAAGTTTTAGCTTTGGAAGAAT | |
| | GCCTAAAAAGACGAAAGTTGGCAAAGCAGCCTGAAACAGTTTCTGTTGCTGAACTCAA | |
| | AAGTCTGTTAGTACTCACAAGGAAACACTTTTTAGATTATTTTGATGCTGTGATTCCT | |
| | AAAATGATTCTAAGAAAGATGGACAAAATTAAAACCTTCAATATATTAAATGATTTTA | |
| | GTCCAGTGGAACCTAATTCCTCAAGTCTAATGGAAACCAATCCTCTGGAATGGCCAGA | |
| | AAGGCATGTTCTTCAAAATTTGGAAACTTTTGAAAAAACTAAACAAAAAATGAGAACT | |
| | GGTTCATTACCTCATTCATCTGAACAGTTGCTGGGCCACAAAGAGGGTCCTCGGGACT | |
| | CAATCACATTGTTGGATGCTAAAGAATTGCTGAAGTACTTTACCTCAGATGGATTACC | |
| | CATTGGAGATCTTCAACCTTTACCGATTCAAAAGGGGGAAAAGACTTTTGTTTTGACA | |
| | CCAGAACTTAGTCCTGGGAAACTTCAGGTCTTACCTTTTGAGAAAGCCTCAGTATGTC | |
| | ATTATCATGGAATTGAATATTGCTTGGATGACCGAAAAGCTTTGGAAAGAGATGGAGG | |
| | ATTTTCTGAACTTCAGTCTCGTCTTATTCGTTATGAAACTCAAACTACCTGCACCAGA | |
| | GAAAGTTTTCCAGTACCTACTGTGTTGAGCCCTCTTCCATCTCCTGTAGTTTCGTCAG | |
| | ATCCTGGAAGTGTCCCTGACGGAGAAGTTTTACAAAATGAACTTCGAACTGAAGTATC | |
| | CCGATTGAAACGGAGATCTAAAGATCTGAATTGCCTTTATCCCAGAAAAAGACTTGTG | |
| | AAATCTGAAAGTTCAGAGTCTCTTCTTTCTCAGACAACTGGTAATAGTAATCACTATC | |
| | ATCATCATGTGACATCCAGAAAGCCACAAACAGAACGGTCCTTACCAGTGACTTGTCC | |
| | ATTGGTTCCAATTCCTAGCTGTGAAACTCCAAAACTTGCTACAAAGACCAGTTCAGGT | |
| | CAAAAAAGTATGCATGAATCAAAAACATCAAGGCAAATTAAGGAATCAAGATCACAGA | |
| | AACACACACGGATACTGAAAGAAGTAGTTACTGAAACCCTGAAGAAACACAGTATTAC | |
| | CGAGACTCATGAATGTTTCACTGCATGCAGCCAGCGTCTCTTTGAAATCTCTAAGTTC | |
| | TATCTAAAGGATCTTAAAACTTCAAGGGGTCTATTTGAAGAAATGAAGAAAACAGCAA | |
| | ACAACAATGCTGTACAGGTGATTGACTGGGTATTAGAAAAGACAAGCAAGAAATGA | |
| ALK- | ATGGGAGCCATCGGGCTCCTGTGGCTCCTGCCGCTGCTGCTTTCCACGGCAGCTGTGG | 24 |
| SLC30A6 | GCTCCGGGATGGGGACCGGCCAGCGCGCGGGCTCCCCAGCTGCGGGGCCGCCGCTGCA | |
| | GCCCCGGGAGCCACTCAGCTACTCGCGCCTGCAGAGGAAGAGTCTGGCAGTTGACTTC | |
| | GTGGTGCCCTCGCTCTTCCGTGTCTACGCCCGGGACCTACTGCTGCCACCATCCTCCT | |
| | CGGAGCTGAAGGCTGGCAGGCCCGAGGCCCGCGGCTCGCTAGCTCTGGACTGCGCCCC | |
| | GCTGCTCAGGTTGCTGGGGCCGGCGCCGGGGGTCTCCTGGACCGCCGGTTCACCAGCC | |
| | CCGGCAGAGGCCCGGACGCTGTCCAGGGTGCTGAAGGGCGGCTCCGTGCGCAAGCTCC | |
| | GGCGTGCCAAGCAGTTGGTGCTGGAGCTGGGCGAGGAGGCGATCTTGGAGGGTTGCGT | |
| | CGGGCCCCCCGGGGAGGCGGCTGTGGGGCTGCTCCAGTTCAATCTCAGCGAGCTGTTC | |
| | AGTTGGTGGATTCGCCAAGGCGAAGGGCGACTGAGGATCCGCCTGATGCCCGAGAAGA | |
| | AGGCGTCGGAAGTGGGCAGAGAGGGAAGGCTGTCCGCGGCAATTCGCGCCTCCCAGCC | |
| | CCGCCTTCTCTTCCAGATCTTCGGGACTGGTCATAGCTCCTTGGAATCACCAACAAAC | |
| | ATGCCTTCTCCTTCTCCTGATTATTTTACATGGAATCTCACCTGGATAATGAAAGACT | |
| | CCTTCCCTTTCCTGTCTCATCGCAGCCGATATGGTCTGGAGTGCAGCTTTGACTTCCC | |
| | CTGTGAGCTGGAGTATTCCCCTCCACTGCATGACCTCAGGAACCAGAGCTGGTCCTGG | |
| | CGCCGCATCCCCTCCGAGGAGGCCTCCCAGATGGACTTGCTGGATGGGCCTGGGGCAG | |
| | AGCGTTCTAAGGAGATGCCCAGAGGCTCCTTTCTCCTTCTCAACACCTCAGCTGACTC | |
| | CAAGCACACCATCCTGAGTCCGTGGATGAGGAGCAGCAGTGAGCACTGCACACTGGCC | |
| | GTCTCGGTGCACAGGCACCTGCAGCCCTCTGGAAGGTACATTGCCCAGCTGCTGCCCC | |
| | ACAACGAGGCTGCAAGAGAGATCCTCCTGATGCCCACTCCAGGGAAGCATGGTTGGAC | |
| | AGTGCTCCAGGGAAGAATCGGGCGTCCAGACAACCCATTTCGAGTGGCCCTGGAATAC | |
| | ATCTCCAGTGGAAACCGCAGCTTGTCTGCAGTGGACTTCTTTGCCCTGAAGAACTGCA | |
| | GTGAAGGAACATCCCCAGGCTCCAAGATGGCCCTGCAGAGCTCCTTCACTTGTTGGAA | |
| | TGGGACAGTCCTCCAGCTTGGGCAGGCCTGTGACTTCCACCAGGACTGTGCCCAGGGA | |
| | GAAGATGAGAGCCAGATGTGCCGGAAACTGCCTGTGGGTTTTTACTGCAACTTTGAAG | |
| | ATGGCTTCTGTGGCTGGACCCAAGGCACACTGTCACCCCACACTCCTCAATGGCAGGT | |
| | CAGGACCCTAAAGGATGCCCGGTTCCAGGACCACCAAGACCATGCTCTATTGCTCAGT | |
| | ACCACTGATGTCCCCGCTTCTGAAAGTGCTACAGTGACCAGTGCTACGTTTCCTGCAC | |
| | CGATCAAGAGCTCTCCATGTGAGCTCCGAATGTCCTGGCTCATTCGTGGAGTCTTGAG | |
| | GGGAAACGTGTCCTTGGTGCTAGTGGAGAACAAAACCGGGAAGGAGCAAGGCAGGATG | |
| | GTCTGGCATGTCGCCGCCTATGAAGGCTTGAGCCTGTGGCAGTGGATGGTGTTGCCTC | |
| | TCCTCGATGTGTCTGACAGGTTCTGGCTGCAGATGGTCGCATGGTGGGGACAAGGATC | |
| | CAGAGCCATCGTGGCTTTTGACAATATCTCCATCAGCCTGGACTGCTACCTCACCATT | |
| | AGCGGAGAGGACAAGATCCTGCAGAATACAGCACCCAAATCAAGAAACCTGTTTGAGA | |
| | GAAACCCAAACAAGGAGCTGAAACCCGGGGAAAATTCACCAAGACAGACCCCCATCTT | |
| | TGACCCTACAGTTCATTGGCTGTTCACCACATGTGGGGCCAGCGGGCCCCATGGCCCC | |
| | ACCCAGGCACAGTGCAACAACGCCTACCAGAACTCCAACCTGAGCGTGGAGGTGGGGA | |
| | GCGAGGGCCCCCTGAAAGGCATCCAGATCTGGAAGGTGCCAGCCACCGACACCTACAG | |
| | CATCTCGGGCTACGGAGCTGCTGGCGGGAAAGGCGGGAAGAACACCATGATGCGGTCC | |
| | CACGGCGTGTCTGTGCTGGGCATCTTCAACCTGGAGAAGGATGACATGCTGTACATCC | |
| | TGGTTGGGCAGCAGGGAGAGGACGCCTGCCCCAGTACAAACCAGTTAATCCAGAAAGT | |
| | CTGCATTGGAGAGAACAATGTGATAGAAGAAGAAATCCGTGTGAACAGAAGCGTGCAT | |
| | GAGTGGGCAGGAGGCGGAGGAGGAGGGGGTGGAGCCACCTACGTATTTAAGATGAAGG | |
| | ATGGAGTGCCGGTGCCCCTGATCATTGCAGCCGGAGGTGGTGGCAGGGCCTACGGGGC | |
| | CAAGACAGACACGTTCCACCCAGAGAGACTGGAGAATAACTCCTCGGTTCTAGGGCTA | |
| | AACGGCAATTCCGGAGCCGCAGGTGGTGGAGGTGGCTGGAATGATAACACTTCCTTGC | |
| | TCTGGGCCGGAAAATCTTTGCAGGAGGGTGCCACCGGAGGACATTCCTGCCCCCAGGC | |
| | CATGAAGAAGTGGGGGTGGGAGACAAGAGGGGGTTTCGGAGGGGGTGGAGGGGGGTGC | |
| | TCCTCAGGTGGAGGAGGCGGAGGATATATAGGCGGCAATGCAGCCTCAAACAATGACC | |
| | CCGAAATGGATGGGGAAGATGGGGTTTCCTTCATCAGTCCACTGGGCATCCTGTACAC | |
| | CCCAGCTTTAAAAGTGATGGAAGGCCACGGGGAAGTGAATATTAAGCATTATCTAAAC | |
| | TGCAGTCACTGTGAGGTAGACGAATGTCACATGGACCCTGAAAGCCACAAGGTCATCT | |
| | GCTTCTGTGACCACGGGACGGTGCTGGCTGAGGATGGCGTCTCCTGCATTGCTTTAAC | |
| | TGCCTATACTTACCTGACCATTTTTGATCTTTTTAGTTTAATGACATGTTTAATAAGT | |
| | TACTGGGTAACATTGAGGAAACCTAGCCCTGTCTATTCATTTGGGTTTGAAAGATTAG | |
| | AAGTCCTGGCTGTATTTGCCTCCACAGTCTTGGCACAGTTGGGAGCTCTCTTTATATT | |
| | AAAAGAAAGTGCAGAACGCTTTTTGGAACAGCCCGAGATACACACGGGAAGATTATTA | |
| | GTTGGTACTTTTGTGGCTCTTTGTTTCAACCTGTTCACGATGCTTTCTATTCGGAATA | |
| | AACCTTTTGCTTATGTCTCAGAAGCTGCTAGTACGAGCTGGCTTCAAGAGCATGTTGC | |
| | AGATCTTAGTCGAAGCTTGTGTGGAATTATTCCGGGACTTAGCAGTATCTTCCTTCCC | |
| | CGAATGAATCCATTTGTTTTGATTGATCTTGCTGGAGCATTTGCTCTTTGTATTACAT | |
| | ATATGCTCATTGAAATTAATAATTATTTTGCCGTAGACACTGCCTCTGCTATAGCTAT | |
| | TGCCTTGATGACATTTGGCACTATGTATCCCATGAGTGTGTACAGTGGGAAAGTCTTA | |
| | CTCCAGACAACACCACCCCATGTTATTGGTCAGTTGGACAAACTCATCAGAGAGGTAT | |
| | CTACCTTAGATGGAGTTTTAGAAGTCCGAAATGAACATTTTTGGACCCTAGGTTTTGG | |
| | CTCATTGGCTGGATCAGTGCATGTAAGAATTCGACGAGATGCCAATGAACAAATGGTT | |
| | CTTGCTCATGTGACCAACAGGCTGTACACTCTAGTGTCTACTCTAACTGTTCAAATTT | |
| | TCAAGGATGACTGGATTAGGCCTGCCTTATTGTCTGGGCCTGTTGCAGCCAATGTCCT | |
| | AAACTTTTCAGATCATCACGTAATCCCAATGCCTCTTTTAAAGGGTACTGATGATTTG | |
| | AACCCAGTTACATCAACTCCAGCTAAACCTAGTAGTCCACCTCCAGAATTTTCATTTA | |
| | ACACTCCTGGGAAAAATGTGAACCCAGTTATTCTTCTAAACACACAAACAAGGCCTTA | |
| | TGGTTTTGGTCTCAATCATGGACACACACCTTACAGCAGCATGCTTAATCAAGGACTT | |
| | GGAGTTCCAGGAATTGGAGCAACTCAAGGATTGAGGACTGGTTTTACAAATATACCAA | |
| | GTAGATATGGAACTAATAATAGAATTGGACAACCAAGACCATGA | |
| ALK- | ATGGGAGCCATCGGGCTCCTGTGGCTCCTGCCGCTGCTGCTTTCCACGGCAGCTGTGG | 25 |
| GMCL1 | GCTCCGGGATGGGGACCGGCCAGCGCGCGGGCTCCCCAGCTGCGGGGCCGCCGCTGCA | |
| | GCCCCGGGAGCCACTCAGCTACTCGCGCCTGCAGAGGAAGAGTCTGGCAGTTGACTTC | |
| | GTGGTGCCCTCGCTCTTCCGTGTCTACGCCCGGGACCTACTGCTGCCACCATCCTCCT | |
| | CGGAGCTGAAGGCTGGCAGGCCCGAGGCCCGCGGCTCGCTAGCTCTGGACTGCGCCCC | |
| | GCTGCTCAGGTTGCTGGGGCCGGCGCCGGGGGTCTCCTGGACCGCCGGTTCACCAGCC | |
| | CCGGCAGAGGCCCGGACGCTGTCCAGGGTGCTGAAGGGCGGCTCCGTGCGCAAGCTCC | |
| | GGCGTGCCAAGCAGTTGGTGCTGGAGCTGGGCGAGGAGGCGATCTTGGAGGGTTGCGT | |
| | CGGGCCCCCCGGGGAGGCGGCTGTGGGGCTGCTCCAGTTCAATCTCAGCGAGCTGTTC | |
| | AGTTGGTGGATTCGCCAAGGCGAAGGGCGACTGAGGATCCGCCTGATGCCCGAGAAGA | |
| | AGGCGTCGGAAGTGGGCAGAGAGGGAAGGCTGTCCGCGGCAATTCGCGCCTCCCAGCC | |
| | CCGCCTTCTCTTCCAGATCTTCGGGACTGGTCATAGCTCCTTGGAATCACCAACAAAC | |
| | ATGCCTTCTCCTTCTCCTGATTATTTTACATGGAATCTCACCTGGATAATGAAAGACT | |
| | CCTTCCCTTTCCTGTCTCATCGCAGCCGATATGGTCTGGAGTGCAGCTTTGACTTCCC | |
| | CTGTGAGCTGGAGTATTCCCCTCCACTGCATGACCTCAGGAACCAGAGCTGGTCCTGG | |
| | CGCCGCATCCCCTCCGAGGAGGCCTCCCAGATGGACTTGCTGGATGGGCCTGGGGCAG | |
| | AGCGTTCTAAGGAGATGCCCAGAGGCTCCTTTCTCCTTCTCAACACCTCAGCTGACTC | |
| | CAAGCACACCATCCTGAGTCCGTGGATGAGGAGCAGCAGTGAGCACTGCACACTGGCC | |
| | GTCTCGGTGCACAGGCACCTGCAGCCCTCTGGAAGGTACATTGCCCAGCTGCTGCCCC | |
| | ACAACGAGGCTGCAAGAGAGATCCTCCTGATGCCCACTCCAGGGAAGCATGGTTGGAC | |
| | AGTGCTCCAGGGAAGAATCGGGCGTCCAGACAACCCATTTCGAGTGGCCCTGGAATAC | |
| | ATCTCCAGTGGAAACCGCAGCTTGTCTGCAGTGGACTTCTTTGCCCTGAAGAACTGCA | |
| | GTGAAGGAACATCCCCAGGCTCCAAGATGGCCCTGCAGAGCTCCTTCACTTGTTGGAA | |
| | TGGGACAGTCCTCCAGCTTGGGCAGGCCTGTGACTTCCACCAGGACTGTGCCCAGGGA | |
| | GAAGATGAGAGCCAGATGTGCCGGAAACTGCCTGTGGGTTTTTACTGCAACTTTGAAG | |
| | ATGGCTTCTGTGGCTGGACCCAAGGCACACTGTCACCCCACACTCCTCAATGGCAGGT | |
| | CAGGACCCTAAAGGATGCCCGGTTCCAGGACCACCAAGACCATGCTCTATTGCTCAGT | |
| | ACCACTGATGTCCCCGCTTCTGAAAGTGCTACAGTGACCAGTGCTACGTTTCCTGCAC | |
| | CGATCAAGAGCTCTCCATGTGAGCTCCGAATGTCCTGGCTCATTCGTGGAGTCTTGAG | |
| | GGGAAACGTGTCCTTGGTGCTAGTGGAGAACAAAACCGGGAAGGAGCAAGGCAGGATG | |
| | GTCTGGCATGTCGCCGCCTATGAAGGCTTGAGCCTGTGGCAGTGGATGGTGTTGCCTC | |
| | TCCTCGATGTGTCTGACAGGTTCTGGCTGCAGATGGTCGCATGGTGGGGACAAGGATC | |
| | CAGAGCCATCGTGGCTTTTGACAATATCTCCATCAGCCTGGACTGCTACCTCACCATT | |
| | AGCGGAGAGGACAAGATCCTGCAGAATACAGCACCCAAATCAAGAAACCTGTTTGAGA | |
| | GAAACCCAAACAAGGAGCTGAAACCCGGGGAAAATTCACCAAGACAGACCCCCATCTT | |
| | TGACCCTACAGTTCATTGGCTGTTCACCACATGTGGGGCCAGCGGGCCCCATGGCCCC | |
| | ACCCAGGCACAGTGCAACAACGCCTACCAGAACTCCAACCTGAGCGTGGAGGTGGGGA | |
| | GCGAGGGCCCCCTGAAAGGCATCCAGATCTGGAAGGTGCCAGCCACCGACACCTACAG | |
| | CATCTCGGGCTACGGAGCTGCTGGCGGGAAAGGCGGGAAGAACACCATGATGCGGTCC | |
| | CACGGCGTGTCTGTGCTGGGCATCTTCAACCTGGAGAAGGATGACATGCTGTACATCC | |
| | TGGTTGGGCAGCAGGGAGAGGACGCCTGCCCCAGTACAAACCAGTTAATCCAGAAAGT | |
| | CTGCATTGGAGAGAACAATGTGATAGAAGAAGAAATCCGTGTGAACAGAAGCGTGCAT | |
| | GAGTGGGCAGGAGGCGGAGGAGGAGGGGGTGGAGCCACCTACGTATTTAAGATGAAGG | |
| | ATGGAGTGCCGGTGCCCCTGATCATTGCAGCCGGAGGTGGTGGCAGGGCCTACGGGGC | |
| | CAAGACAGACACGTTCCACCCAGAGAGACTGGAGAATAACTCCTCGGTTCTAGGGCTA | |
| | AACGGCAATTCCGGAGCCGCAGGTGGTGGAGGTGGCTGGAATGATAACACTTCCTTGC | |
| | TCTGGGCCGGAAAATCTTTGCAGGAGGGTGCCACCGGAGGACATTCCTGCCCCCAGGC | |
| | CATGAAGAAGTGGGGGTGGGAGACAAGAGGGGGTTTCGGAGGGGGTGGAGGGGGGTGC | |
| | TCCTCAGGTGGAGGAGGCGGAGGATATATAGGCGGCAATGCAGCCTCAAACAATGACC | |
| | CCGAAATGGATGGGGAAGATGGGGTTTCCTTCATCAGTCCACTGGGCATCCTGTACAC | |
| | CCCAGCTTTAAAAGTGATGGAAGGCCACGGGGAAGTGAATATTAAGCATTATCTAAAC | |
| | TGCAGTCACTGTGAGGTAGACGAATGTCACATGGACCCTGAAAGCCACAAGGTCATCT | |
| | GCTTCTGTGACCACGGGACGGTGCTGGCTGAGGATGGCGTCTCCTGCATTGTGTCACC | |
| | CACCCCGGAGCCACACCTGCCACTCTCGCTGATCCTCTCTGTGGTGACCTCTGCCCTC | |
| | GTGGCCGCCCTGGTCCTGGCTTTCTCCGGCATCATGATTGTGTACCGCCGGAAGCACC | |
| | AGGAGCTGCAAGCCATGCAGATGGAGCTGCAGAGCCCTGAGTACAAGCTGAGCAAGCT | |
| | CCGCACCTCGACCATCATGACCGACTACAACCCCAACTACTGCTTTGCTGGCAAGACC | |
| | TCCTCCATCAGTGACCTGAAGGAGGTGCCGCGGAAAAACATCACCCTCATTCGGGGTC | |
| | TGGGCCATGGCGCCTTTGGGGAGGTGTATGAAGGCCAGGTGTCCGGAATGCCCAACGA | |
| | CCCAAGCCCCCTGCAAGTGGCTGTGAAGACGCTGCCTGAAGTGTGCTCTGAACAGGAC | |
| | GAACTGGATTTCCTCATGGAAGCCCTGATCATCAGCAAATTCAACCACCAGAACATTG | |
| | TTCGCTGCATTGGGGTGAGCCTGCAATCCCTGCCCCGGTTCATCCTGCTGGAGCTCAT | |
| | GGCGGGGGGAGACCTCAAGTCCTTCCTCCGAGAGACCCGCCCTCGCCCGAGCCAGCCC | |
| | TCCTCCCTGGCCATGCTGGACCTTCTGCACGTGGCTCGGGACATTGCCTGTGGCTGTC | |
| | AGTATTTGGAGGAAAACCACTTCATCCACCGAGACATTGCTGCCAGAAACTGCCTCTT | |
| | GACCTGTCCAGGCCCTGGAAGAGTGGCCAAGATTGGAGACTTCGGGATGGCCCGAGAC | |
| | ATCTACAGGGCGAGCTACTATAGAAAGGGAGGCTGTGCCATGCTGCCAGITAAGTGGA | |
| | TGCCCCCAGAGGCCTTCATGGAAGGAATATTCACTTCTAAAACAGACACATGGTCCTT | |
| | TGGAGTGCTGCTATGGGAAATCTTTTCTCTTGGATATATGCCATACCCCAGCAAAAGC | |
| | AACCAGGAAGTTCTGGAGTTTGTCACCAGTGGAGGCCGGATGGACCCACCCAAGAACT | |
| | GCCCTGGGCCTGTTATAAATGTCATGAAACAGCTCATTGGTTCATCTAACTTATTTGT | |
| | GATGCAAGTGGAGATGGATATATACACTGCTCTAAAAAAGTGGATGTTCCTTCAACTT | |
| | GTGCCTTCTTGGAATGGATCTTTAAAACAGCTTTTGACAGAAACAGATGTCTGGTTTT | |
| | CTAAACAGAGGAAAGATTTTGAAGGTATGGCCTTTCTTGAAACTGAACAAGGAAAACC | |
| | ATTTGTGTCAGTATTCAGACATTTAAGGTTACAATATATTATCAGTGATCTGGCTTCT | |
| | GCAAGAATTATTGAACAAGATGCTGTAGTACCTTCAGAATGGCTCTCTTCTGTGTATA | |
| | AACAGCAGTGGTTTGCTATGCTGCGGGCAGAACAGGACAGTGAGGTGGGGCCTCAAGA | |
| | AATCAATAAAGAAGAACTAGAGGGAAACAGCATGAGGTGTGGTAGAAAGCTTGCCAAA | |
| | GATGGTGAATACTGCTGGCGTTGGACAGGTTTTAACTTCGGCTTCGACCTACTTGTAA | |
| | CTTACACCAATCGATACATCATTTTCAAACGCAATACACTGAATCAGCCATGTAGCGG | |
| | ATCTGTCAGTTTACAGCCTCGAAGGAGCATAGCATTTAGATTACGTTTGGCTTCTTTT | |
| | GATAGTAGTGGAAAACTAATATGTAGTAGAACAACTGGCTATCAAATACTTACACTTG | |
| | AAAAGGATCAGGAACAAGTGGTGATGAACTTGGACAGCAGGCTTCTGATCTTCCCTTT | |
| | ATATATCTGCTGTAACTTCTTGTATATATCACCAGAAAAAAAGAATTGA | |
| ALK- | ATGGGAGCCATCGGGCTCCTGTGGCTCCTGCCGCTGCTGCTTTCCACGGCAGCTGTGG | 26 |
| AGAP1 | GCTCCGGGATGGGGACCGGCCAGCGCGCGGGCTCCCCAGCTGCGGGGCCGCCGCTGCA | |
| | GCCCCGGGAGCCACTCAGCTACTCGCGCCTGCAGAGGAAGAGTCTGGCAGTTGACTTC | |
| | GTGGTGCCCTCGCTCTTCCGTGTCTACGCCCGGGACCTACTGCTGCCACCATCCTCCT | |
| | CGGAGCTGAAGGCTGGCAGGCCCGAGGCCCGCGGCTCGCTAGCTCTGGACTGCGCCCC | |
| | GCTGCTCAGGTTGCTGGGGCCGGCGCCGGGGGTCTCCTGGACCGCCGGTTCACCAGCC | |
| | CCGGCAGAGGCCCGGACGCTGTCCAGGGTGCTGAAGGGCGGCTCCGTGCGCAAGCTCC | |
| | GGCGTGCCAAGCAGTTGGTGCTGGAGCTGGGCGAGGAGGCGATCTTGGAGGGTTGCGT | |
| | CGGGCCCCCCGGGGAGGCGGCTGTGGGGCTGCTCCAGTTCAATCTCAGCGAGCTGTTC | |
| | AGTTGGTGGATTCGCCAAGGCGAAGGGCGACTGAGGATCCGCCTGATGCCCGAGAAGA | |
| | AGGCGTCGGAAGTGGGCAGAGAGGGAAGGCTGTCCGCGGCAATTCGCGCCTCCCAGCC | |
| | CCGCCTTCTCTTCCAGATCTTCGGGACTGGTCATAGCTCCTTGGAATCACCAACAAAC | |
| | ATGCCTTCTCCTTCTCCTGATTATTTTACATGGAATCTCACCTGGATAATGAAAGACT | |
| | CCTTCCCTTTCCTGTCTCATCGCAGCCGATATGGTCTGGAGTGCAGCTTTGACTTCCC | |
| | CTGTGAGCTGGAGTATTCCCCTCCACTGCATGACCTCAGGAACCAGAGCTGGTCCTGG | |
| | CGCCGCATCCCCTCCGAGGAGGCCTCCCAGATGGACTTGCTGGATGGGCCTGGGGCAG | |
| | AGCGTTCTAAGGAGATGCCCAGAGGCTCCTTTCTCCTTCTCAACACCTCAGCTGACTC | |
| | CAAGCACACCATCCTGAGTCCGTGGATGAGGAGCAGCAGTGAGCACTGCACACTGGCC | |
| | GTCTCGGTGCACAGGCACCTGCAGCCCTCTGGAAGGTACATTGCCCAGCTGCTGCCCC | |
| | ACAACGAGGCTGCAAGAGAGATCCTCCTGATGCCCACTCCAGGGAAGCATGGTTGGAC | |
| | AGTGCTCCAGGGAAGAATCGGGCGTCCAGACAACCCATTTCGAGTGGCCCTGGAATAC | |
| | ATCTCCAGTGGAAACCGCAGCTTGTCTGCAGTGGACTTCTTTGCCCTGAAGAACTGCA | |
| | GTGAAGGAACATCCCCAGGCTCCAAGATGGCCCTGCAGAGCTCCTTCACTTGTTGGAA | |
| | TGGGACAGTCCTCCAGCTTGGGCAGGCCTGTGACTTCCACCAGGACTGTGCCCAGGGA | |
| | GAAGATGAGAGCCAGATGTGCCGGAAACTGCCTGTGGGTTTTTACTGCAACTTTGAAG | |
| | ATGGCTTCTGTGGCTGGACCCAAGGCACACTGTCACCCCACACTCCTCAATGGCAGGT | |
| | CAGGACCCTAAAGGATGCCCGGTTCCAGGACCACCAAGACCATGCTCTATTGCTCAGT | |
| | ACCACTGATGTCCCCGCTTCTGAAAGTGCTACAGTGACCAGTGCTACGTTTCCTGCAC | |
| | CGATCAAGAGCTCTCCATGTGAGCTCCGAATGTCCTGGCTCATTCGTGGAGTCTTGAG | |
| | GGGAAACGTGTCCTTGGTGCTAGTGGAGAACAAAACCGGGAAGGAGCAAGGCAGGATG | |
| | GTCTGGCATGTCGCCGCCTATGAAGGCTTGAGCCTGTGGCAGTGGATGGTGTTGCCTC | |
| | TCCTCGATGTGTCTGACAGGTTCTGGCTGCAGATGGTCGCATGGTGGGGACAAGGATC | |
| | CAGAGCCATCGTGGCTTTTGACAATATCTCCATCAGCCTGGACTGCTACCTCACCATT | |
| | AGCGGAGAGGACAAGATCCTGCAGAATACAGCACCCAAATCAAGAAACCTGTTTGAGA | |
| | GAAACCCAAACAAGGAGCTGAAACCCGGGGAAAATTCACCAAGACAGACCCCCATCTT | |
| | TGACCCTACAGTTCATTGGCTGTTCACCACATGTGGGGCCAGCGGGCCCCATGGCCCC | |
| | ACCCAGGCACAGTGCAACAACGCCTACCAGAACTCCAACCTGAGCGTGGAGGTGGGGA | |
| | GCGAGGGCCCCCTGAAAGGCATCCAGATCTGGAAGGTGCCAGCCACCGACACCTACAG | |
| | CATCTCGGGCTACGGAGCTGCTGGCGGGAAAGGCGGGAAGAACACCATGATGCGGTCC | |
| | CACGGCGTGTCTGTGCTGGGCATCTTCAACCTGGAGAAGGATGACATGCTGTACATCC | |
| | TGGTTGGGCAGCAGGGAGAGGACGCCTGCCCCAGTACAAACCAGTTAATCCAGAAAGT | |
| | CTGCATTGGAGAGAACAATGTGATAGAAGAAGAAATCCGTGTGAACAGAAGCGTGCAT | |
| | GAGTGGGCAGGAGGCGGAGGAGGAGGGGGTGGAGCCACCTACGTATTTAAGATGAAGG | |
| | ATGGAGTGCCGGTGCCCCTGATCATTGCAGCCGGAGGTGGTGGCAGGGCCTACGGGGC | |
| | CAAGACAGACACGTTCCACCCAGAGAGACTGGAGAATAACTCCTCGGTTCTAGGGCTA | |
| | AACGGCAATTCCGGAGCCGCAGGTGGTGGAGGTGGCTGGAATGATAACACTTCCTTGC | |
| | TCTGGGCCGGAAAATCTTTGCAGGAGGGTGCCACCGGAGGACATTCCTGCCCCCAGGC | |
| | CATGAAGAAGTGGGGGTGGGAGACAAGAGGGGGTTTCGGAGGGGGTGGAGGGGGGTGC | |
| | TCCTCAGGTGGAGGAGGCGGAGGATATATAGGCGGCAATGCAGCCTCAAACAATGACC | |
| | CCGAAATGGATGGGGAAGATGGGGTTTCCTTCATCAGTCCACTGGGCATCCTGTACAC | |
| | CCCAGCTTTAAAAGTGATGGAAGGCCACGGGGAAGTGAATATTAAGCATTATCTAAAC | |
| | TGCAGTCACTGTGAGGTAGACGAATGTCACATGGACCCTGAAAGCCACAAGGTCATCT | |
| | GCTTCTGTGACCACGGGACGGTGCTGGCTGAGGATGGCGTCTCCTGCATTGTGTCACC | |
| | CACCCCGGAGCCACACCTGCCACTCTCGCTGATCCTCTCTGTGGTGACCTCTGCCCTC | |
| | GTGGCCGCCCTGGTCCTGGCTTTCTCCGGCATCATGATTGTTGCCCAGAAGATTGTTG | |
| | CCACAAGGAAGAAGCAGCAGCTGTCCATAGGACCCTGCAAGTCGCTACCTAATTCTCC | |
| | CAGCCATTCCTCCGTCTGTTCCGCGCAGGTGTCTGCCGTGCACATCAGCCAGACAAGT | |
| | AATGGAGGTGGGAGTTTAAGCGACTATTCCTCCTCCGTTCCATCGACTCCCAGCACCA | |
| | GCCAGAAGGAACTTCGGATCGATGTTCCTCCCACTGCCAACACGCCCACGCCCGTTCG | |
| | CAAGCAGTCTAAGCGCCGGTCCAACCTGTTCACCTCTCGGAAAGGGAGCGACCCAGAC | |
| | AAAGAGAAGAAAGGCCTGGAGAGTCGTGCGGACAGCATTGGGAGCGGCCGAGCCATCC | |
| | CAATTAAACAGGGCATGCTGTTGAAGCGAAGTGGCAAATCGTTGAATAAAGAGTGGAA | |
| | AAAGAAATATGTCACCCTGTGTGACAATGGCGTGCTGACCTATCATCCCAGTTTACAT | |
| | GATTACATGCAGAATGTTCATGGTAAGGAGATTGACCTTCTGAGAACCACTGTGAAAG | |
| | TCCCAGGGAAGAGGCCACCCCGAGCCACGTCAGCCTGCGCACCCATCTCCAGCCCTAA | |
| | AACCAATGGCCTATCCAAGGACATGAGCAGTTTACACATCTCACCCAATTCAGACACA | |
| | GGGCTGGGTGACTCCGTATGCTCCAGCCCCAGTATCTCCAGCACCACCAGCCCCAAGC | |
| | TCGACCCGCCCCCCTCCCCTCACGCCAACAGAAAGAAGCACCGAAGGAAGAAAAGCAC | |
| | TAGCAACTTCAAAGCCGACGGCCTGTCCGGCACTGCTGAAGAACAAGAAGAAAATTTT | |
| | GAGTTTATCATTGTGTCCCTCACTGGCCAAACATGGCACTTTGAAGCCACGACGTATG | |
| | AGGAGCGGGACGCCTGGGTCCAAGCCATCGAGAGCCAGATCCTGGCCAGCCTGCAGTC | |
| | GTGCGAGAGCAGCAAGAACAAGTCCCGGCTGACGAGCCAGAGCGAGGCCATGGCCCTG | |
| | CAGTCGATCCGGAACATGCGCGGGAACTCCCACTGTGTGGACTGCGAGACCCAGAATC | |
| | CCAACTGGGCCAGTTTGAACTTGGGAGCCCTCATGTGCATCGAATGCTCAGGGATCCA | |
| | CCGGAATCTTGGCACCCACCTTTCCCGAGTCCGATCTCTGGACCTGGATGACTGGCCA | |
| | GTCGAGCTCATCAAGGTGATGTCATCCATCGGGAACGAGCTAGCCAACAGCGTCTGGG | |
| | AAGAGAGCAGCCAGGGGCGGACGAAACCATCGGTAGACTCCACAAGGGAAGAGAAGGA | |
| | ACGGTGGATCCGTGCCAAGTACGAGCAGAAGCTCTTCCTGGCCCCGCTGCCCTGCACG | |
| | GAGCTGTCCCTGGGCCAGCACCTGCTGCGGGCCACCGCCGACGAGGACCTGCGGACGG | |
| | CCATCCTGCTGCTGGCACACGGCTCCCGGGACGAGGTGAACGAGACCTGCGGGGAGGG | |
| | AGACGGCCGCACGGCGCTGCATCTGGCCTGCCGCAAGGGGAATGTGGTCCTGGCGCAG | |
| | CTCCTGATCTGGTACGGAGTGGACGTCACGGCCCGAGATGCCCACGGGAACACAGCTC | |
| | TGGCCTACGCCCGGCAGGCCTCCAGCCAGGAGTGCATCGACGTGCTGCTGCAGTACGG | |
| | CTGCCCCGACGAGCGCTTCGTGCTCATGGCCACCCCTAACCTGTCCAGGAGAAACAAT | |
| | AACCGGAACAACAGCAGTGGGAGGGTGCCCACCATCATCTGA | |
| ALK- | ATGGGAGCCATCGGGCTCCTGTGGCTCCTGCCGCTGCTGCTTTCCACGGCAGCTGTGG | 27 |
| ZNF454 | GCTCCGGGATGGGGACCGGCCAGCGCGCGGGCTCCCCAGCTGCGGGGCCGCCGCTGCA | |
| | GCCCCGGGAGCCACTCAGCTACTCGCGCCTGCAGAGGAAGAGTCTGGCAGTTGACTTC | |
| | GTGGTGCCCTCGCTCTTCCGTGTCTACGCCCGGGACCTACTGCTGCCACCATCCTCCT | |
| | CGGAGCTGAAGGCTGGCAGGCCCGAGGCCCGCGGCTCGCTAGCTCTGGACTGCGCCCC | |
| | GCTGCTCAGGTTGCTGGGGCCGGCGCCGGGGGTCTCCTGGACCGCCGGTTCACCAGCC | |
| | CCGGCAGAGGCCCGGACGCTGTCCAGGGTGCTGAAGGGCGGCTCCGTGCGCAAGCTCC | |
| | GGCGTGCCAAGCAGTTGGTGCTGGAGCTGGGCGAGGAGGCGATCTTGGAGGGTTGCGT | |
| | CGGGCCCCCCGGGGAGGCGGCTGTGGGGCTGCTCCAGTTCAATCTCAGCGAGCTGTTC | |
| | AGTTGGTGGATTCGCCAAGGCGAAGGGCGACTGAGGATCCGCCTGATGCCCGAGAAGA | |
| | AGGCGTCGGAAGTGGGCAGAGAGGGAAGGCTGTCCGCGGCAATTCGCGCCTCCCAGCC | |
| | CCGCCTTCTCTTCCAGATCTTCGGGACTGGTCATAGCTCCTTGGAATCACCAACAAAC | |
| | ATGCCTTCTCCTTCTCCTGATTATTTTACATGGAATCTCACCTGGATAATGAAAGACT | |
| | CCTTCCCTTTCCTGTCTCATCGCAGCCGATATGGTCTGGAGTGCAGCTTTGACTTCCC | |
| | CTGTGAGCTGGAGTATTCCCCTCCACTGCATGACCTCAGGAACCAGAGCTGGTCCTGG | |
| | CGCCGCATCCCCTCCGAGGAGGCCTCCCAGATGGACTTGCTGGATGGGCCTGGGGCAG | |
| | AGCGTTCTAAGGAGATGCCCAGAGGCTCCTTTCTCCTTCTCAACACCTCAGCTGACTC | |
| | CAAGCACACCATCCTGAGTCCGTGGATGAGGAGCAGCAGTGAGCACTGCACACTGGCC | |
| | GTCTCGGTGCACAGGCACCTGCAGCCCTCTGGAAGGTACATTGCCCAGCTGCTGCCCC | |
| | ACAACGAGGCTGCAAGAGAGATCCTCCTGATGCCCACTCCAGGGAAGCATGGTTGGAC | |
| | AGTGCTCCAGGGAAGAATCGGGCGTCCAGACAACCCATTTCGAGTGGCCCTGGAATAC | |
| | ATCTCCAGTGGAAACCGCAGCTTGTCTGCAGTGGACTTCTTTGCCCTGAAGAACTGCA | |
| | GTGAAGGAACATCCCCAGGCTCCAAGATGGCCCTGCAGAGCTCCTTCACTTGTTGGAA | |
| | TGGGACAGTCCTCCAGCTTGGGCAGGCCTGTGACTTCCACCAGGACTGTGCCCAGGGA | |
| | GAAGATGAGAGCCAGATGTGCCGGAAACTGCCTGTGGGTTTTTACTGCAACTTTGAAG | |
| | ATGGCTTCTGTGGCTGGACCCAAGGCACACTGTCACCCCACACTCCTCAATGGCAGGT | |
| | CAGGACCCTAAAGGATGCCCGGTTCCAGGACCACCAAGACCATGCTCTATTGCTCAGT | |
| | ACCACTGATGTCCCCGCTTCTGAAAGTGCTACAGTGACCAGTGCTACGTTTCCTGCAC | |
| | CGATCAAGAGCTCTCCATGTGAGCTCCGAATGTCCTGGCTCATTCGTGGAGTCTTGAG | |
| | GGGAAACGTGTCCTTGGTGCTAGTGGAGAACAAAACCGGGAAGGAGCAAGGCAGGATG | |
| | GTCTGGCATGTCGCCGCCTATGAAGGCTTGAGCCTGTGGCAGTGGATGGTGTTGCCTC | |
| | TCCTCGATGTGTCTGACAGGTTCTGGCTGCAGATGGTCGCATGGTGGGGACAAGGATC | |
| | CAGAGCCATCGTGGCTTTTGACAATATCTCCATCAGCCTGGACTGCTACCTCACCATT | |
| | AGCGGAGAGGACAAGATCCTGCAGAATACAGCACCCAAATCAAGAAACCTGTTTGAGA | |
| | GAAACCCAAACAAGGAGCTGAAACCCGGGGAAAATTCACCAAGACAGACCCCCATCTT | |
| | TGACCCTACAGTTCATTGGCTGTTCACCACATGTGGGGCCAGCGGGCCCCATGGCCCC | |
| | ACCCAGGCACAGTGCAACAACGCCTACCAGAACTCCAACCTGAGCGTGGAGGTGGGGA | |
| | GCGAGGGCCCCCTGAAAGGCATCCAGATCTGGAAGGTGCCAGCCACCGACACCTACAG | |
| | CATCTCGGGCTACGGAGCTGCTGGCGGGAAAGGCGGGAAGAACACCATGATGCGGTCC | |
| | CACGGCGTGTCTGTGCTGGGCATCTTCAACCTGGAGAAGGATGACATGCTGTACATCC | |
| | TGGTTGGGCAGCAGGGAGAGGACGCCTGCCCCAGTACAAACCAGTTAATCCAGAAAGT | |
| | CTGCATTGGAGAGAACAATGTGATAGAAGAAGAAATCCGTGTGAACAGAAGCGTGCAT | |
| | GAGTGGGCAGGAGGCGGAGGAGGAGGGGGTGGAGCCACCTACGTATTTAAGATGAAGG | |
| | ATGGAGTGCCGGTGCCCCTGATCATTGCAGCCGGAGGTGGTGGCAGGGCCTACGGGGC | |
| | CAAGACAGACACGTTCCACCCAGAGAGACTGGAGAATAACTCCTCGGTTCTAGGGCTA | |
| | AACGGCAATTCCGGAGCCGCAGGTGGTGGAGGTGGCTGGAATGATAACACTTCCTTGC | |
| | TCTGGGCCGGAAAATCTTTGCAGGAGGGTGCCACCGGAGGACATTCCTGCCCCCAGGC | |
| | CATGAAGAAGTGGGGGTGGGAGACAAGAGGGGGTTTCGGAGGGGGTGGAGGGGGGTGC | |
| | TCCTCAGGTGGAGGAGGCGGAGGATATATAGGCGGCAATGCAGCCTCAAACAATGACC | |
| | CCGAAATGGATGGGGAAGATGGGGTTTCCTTCATCAGTCCACTGGGCATCCTGTACAC | |
| | CCCAGCTTTAAAAGTGATGGAAGGCCACGGGGAAGTGAATATTAAGCATTATCTAAAC | |
| | TGCAGTCACTGTGAGGTAGACGAATGTCACATGGACCCTGAAAGCCACAAGGTCATCT | |
| | GCTTCTGTGACCACGGGACGGTGCTGGCTGAGGATGGCGTCTCCTGCATTGTGTCACC | |
| | CACCCCGGAGCCACACCTGCCACTCTCGCTGATCCTCTCTGTGGTGACCTCTGCCCTC | |
| | GTGGCCGCCCTGGTCCTGGCTTTCTCCGGCATCATGATTGACTGGATGACTATGCCTG | |
| | CCAGTAAGAAATCTACTGTCAAGGCAGAGATTCCTGAAGAAGAATTGGATCAATGGAC | |
| | AATAAAGGAAAGATTCAGTAGCAGTAGTCACTGGAAGTGTGCTAGCCTGCTGGAGTGG | |
| | CAATGTGGAGGCCAGGAGATCAGTTTGCAGCGAGTGGTACTCACTCACCCCAACACCC | |
| | CATCACAGGAATGTGATGAATCCGGGAGCACTATGAGCTCATCTCTTCACAGTGATCA | |
| | AAGTCAGGGATTTCAACCTAGCAAAAATGCCTTTGAGTGTAGTGAGTGTGGAAAAGTC | |
| | TTCTCTAAGAGTTCAACTCTTAATAAACATCAGAAAATTCATAATGAAAAAAATGCAA | |
| | ATCAGAAAATTCATATTAAGGAGAAAAGATATGAATGTAGAGAATGTGGGAAAGCCTT | |
| | TCACCAGAGTACGCACCTTATCCATCACCAAAGAATTCACACTGGCGAGAAACCCTAT | |
| | GAATGTAAGGAATGTGGCAAGGCCTTCTCAGTGAGCTCCTCACTTACGTACCATCAGA | |
| | AAATTCATACTGGAGAGAAGCCTTTTGAATGCAACTTATGTGGAAAAGCTTTTATCCG | |
| | AAATATACACCTTGCCCATCATCATAGAATACATACTGGAGAGAAACCTTTTAAATGT | |
| | AACATTTGTGAAAAAGCCTTTGTGTGCAGGGCACACCTTACCAAACACCAGAATATCC | |
| | ACAGTGGAGAGAAACCCTATAAATGCAATGAATGTGGAAAAGCCTTTAATCAGAGTAC | |
| | AAGTTTCCTTCAGCATCAGAGAATTCACACTGGAGAGAAACCCTTTGAATGTAATGAA | |
| | TGTGGGAAGGCCTTCAGGGTGAACTCTTCCCTTACTGAACATCAGAGAATTCATACTG | |
| | GAGAGAAACCTTATAAATGTAATGAATGTGGGAAAGCTTTCAGGGATAATTCATCCTT | |
| | TGCACGACATCGGAAAATTCACACTGGAGAGAAACCTTACAGATGTGGCTTGTGTGAG | |
| | AAAGCCTTTCGGGACCAATCAGCACTAGCCCAACATCAGAGAATTCATACTGGGGAAA | |
| | AACCTTATACATGTAACATATGTGAAAAAGCCTTCAGTGACCATTCAGCCCTTACCCA | |
| | ACATAAGAGAATTCATACTAGGGAAAAACCTTACAAATGTAAAATCTGTGAGAAAGCC | |
| | TTTATCCGAAGCACTCACCTGACTCAACATCAGAGGATTCACACAGGAGAGAAACCCT | |
| | ATAAATGTAATAAATGTGGGAAAGCTTTTAACCAGACTGCAAACCTCATTCAGCATCA | |
| | GAGACATCATATTGGAGAGAAGTGA | |
| ALK- | ATGGAGCAGTCGCCGCCGCCGGCGCCCGAGCCGACCCAAGGGCCGACCCCCGCAAGGA | 28 |
| TTC28 | GCCGAAGGCGGCGGGAGCCAGAGTCGCCGCCGGCGTCGGCGCCGACAAACCAGTTAAT | |
| | CCAGAAAGTCTGCATTGGAGAGAACAATGTGATAGAAGAAGAAATCCGTGTGAACAGA | |
| | AGCGTGCATGAGTGGGCAGGAGGCGGAGGAGGAGGGGGTGGAGCCACCTACGTATTTA | |
| | AGATGAAGGATGGAGTGCCGGTGCCCCTGATCATTGCAGCCGGAGGTGGTGGCAGGGC | |
| | CTACGGGGCCAAGACAGACACGTTCCACCCAGAGAGACTGGAGAATAACTCCTCGGTT | |
| | CTAGGGCTAAACGGCAATTCCGGAGCCGCAGGTGGTGGAGGTGGCTGGAATGATAACA | |
| | CTTCCTTGCTCTGGGCCGGAAAATCTTTGCAGGAGGGTGCCACCGGAGGACATTCCTG | |
| | CCCCCAGGCCATGAAGAAGTGGGGGTGGGAGACAAGAGGGGGTTTCGGAGGGGGTGGA | |
| | GGGGGGTGCTCCTCAGGTGGAGGAGGCGGAGGATATATAGGCGGCAATGCAGCCTCAA | |
| | ACAATGACCCCGAAATGGATGGGGAAGATGGGGTTTCCTTCATCAGTCCACTGGGCAT | |
| | CCTGTACACCCCAGCTTTAAAAGTGATGGAAGGCCACGGGGAAGTGAATATTAAGCAT | |
| | TATCTAAACTGCAGTCACTGTGAGGTAGACGAATGTCACATGGACCCTGAAAGCCACA | |
| | AGGTCATCTGCTTCTGTGACCACGGGACGGTGCTGGCTGAGGATGGCGTCTCCTGCAT | |
| | TGTGTCACCCACCCCGGAGCCACACCTGCCACTCTCGCTGATCCTCTCTGTGGTGACC | |
| | TCTGCCCTCGTGGCCGCCCTGGTCCTGGCTTTCTCCGGCATCATGATTGTGTACCGCC | |
| | GGAAGCACCAGGAGCTGCAAGCCATGCAGATGGAGCTGCAGAGCCCTGAGTACAAGCT | |
| | GAGCAAGCTCCGCACCTCGACCATCATGACCGACTACAACCCCAACTACTGCTTTGCT | |
| | GGCAAGACCTCCTCCATCAGTGACCTGAAGGAGGTGCCGCGGAAAAACATCACCCTCA | |
| | TTCGGGGTCTGGGCCATGGCGCCTTTGGGGAGGTGTATGAAGGCCAGGTGTCCGGAAT | |
| | GCCCAACGACCCAAGCCCCCTGCAAGTGGCTGTGAAGACGCTGCCTGAAGTGTGCTCT | |
| | GAACAGGACGAACTGGATTTCCTCATGGAAGCCCTGATCATCAGCAAATTCAACCACC | |
| | AGAACATTGTTCGCTGCATTGGGGTGAGCCTGCAATCCCTGCCCCGGTTCATCCTGCT | |
| | GGAGCTCATGGCGGGGGGAGACCTCAAGTCCTTCCTCCGAGAGACCCGCCCTCGCCCG | |
| | AGCCAGCCCTCCTCCCTGGCCATGCTGGACCTTCTGCACGTGGCTCGGGACATTGCCT | |
| | GTGGCTGTCAGTATTTGGAGGAAAACCACTTCATCCACCGAGACATTGCTGCCAGAAA | |
| | CTGCCTCTTGACCTGTCCAGGCCCTGGAAGAGTGGCCAAGATTGGAGACTTCGGGATG | |
| | GCCCGAGACATCTACAGGGCGAGCTACTATAGAAAGGGAGGCTGTGCCATGCTGCCAG | |
| | TTAAGTGGATGCCCCCAGAGGCCTTCATGGAAGGAATATTCACTTCTAAAACAGACAC | |
| | ATGGTCCTTTGGAGTGCTGCTATGGGAAATCTTTTCTCTTGGATATATGCCATACCCC | |
| | AGCAAAAGCAACCAGGAAGTTCTGGAGTTTGTCACCAGTGGAGGCCGGATGGACCCAC | |
| | CCAAGAACTGCCCTGGGCCTGTATACCGGATAATGACTCAGTGCTGGCAACATCAGCC | |
| | TGAAGACAGGCCCAACTTTGCCATCATTTTGGAGAGGATTGAATACTGCACCCAGGAC | |
| | CCGGATGTAATCAACACCGCTTTGCCGATAGAATATGGTCCACTTGTGGAAGAGGAAG | |
| | AGAAAGTGCCTGTGAGGCCCAAGGACCCTGAGGGGGTTCCTCCTCTCCTGGTCTCTCA | |
| | ACAGGCAAAACGGGAGGAGGAGCGCAGCCCAGCTGCCCCACCACCTCTGCCTACCACC | |
| | TCCTCTGGCAAGGCTGCAAAGAAACCCACAGCTGCAGAGATCTCTGTTCGAGTCCCTA | |
| | GAGGGCCGGCCGTGGAAGGGGGACACGTGAATATGGCATTCTCTCAGTCCAACCCTCC | |
| | TTCGGAGTTGCACAAGGTCCACGGATCCAGAAACAAGCCCACCAGCTTGTGGAACCCA | |
| | ACGTACGGCTCCTGGTTTACAGAGAAACCCACCAAAAAGAATAATCCTATAGCAAAGA | |
| | AGGAGCCACACGACAGGGGTAACCTGGGGCTGGAGGGAAGCTGTACTGTCCCACCTAA | |
| | CGTTGCAACTGGGAGACTTCCGGGGGCCTCACTGCTCCTAGAGCCCTCTTCGCTGACT | |
| | GCCAATATGAAGGAGGTACCTCTGTTCAGGCTACGTCACTTCCCTTGTGGGAATGTCA | |
| | ATTACGGCTACCAGCAACAGGGCTTGCCCTTAGAAGCCGCTACTGCCCCTGGAGCTGG | |
| | TCATTACGAGGATACCATTCTGAAAAGCAAGAATAGCATGAACCAGCCTGGGCCCTGA | |
| ALK-NINJ2 | ATGGAATCAGCAAGAGAAAACATCGACCTTCAAACAAACCAGTTAATCCAGAAAGTCT | 29 |
| | GCATTGGAGAGAACAATGTGATAGAAGAAGAAATCCGTGTGAACAGAAGCGTGCATGA | |
| | GTGGGCAGGAGGCGGAGGAGGAGGGGGTGGAGCCACCTACGTATTTAAGATGAAGGAT | |
| | GGAGTGCCGGTGCCCCTGATCATTGCAGCCGGAGGTGGTGGCAGGGCCTACGGGGCCA | |
| | AGACAGACACGTTCCACCCAGAGAGACTGGAGAATAACTCCTCGGTTCTAGGGCTAAA | |
| | CGGCAATTCCGGAGCCGCAGGTGGTGGAGGTGGCTGGAATGATAACACTTCCTTGCTC | |
| | TGGGCCGGAAAATCTTTGCAGGAGGGTGCCACCGGAGGACATTCCTGCCCCCAGGCCA | |
| | TGAAGAAGTGGGGGTGGGAGACAAGAGGGGGTTTCGGAGGGGGTGGAGGGGGGTGCTC | |
| | CTCAGGTGGAGGAGGCGGAGGATATATAGGCGGCAATGCAGCCTCAAACAATGACCCC | |
| | GAAATGGATGGGGAAGATGGGGTTTCCTTCATCAGTCCACTGGGCATCCTGTACACCC | |
| | CAGCTTTAAAAGTGATGGAAGGCCACGGGGAAGTGAATATTAAGCATTATCTAAACTG | |
| | CAGTCACTGTGAGGTAGACGAATGTCACATGGACCCTGAAAGCCACAAGGTCATCTGC | |
| | TTCTGTGACCACGGGACGGTGCTGGCTGAGGATGGCGTCTCCTGCATTGTGTCACCCA | |
| | CCCCGGAGCCACACCTGCCACTCTCGCTGATCCTCTCTGTGGTGACCTCTGCCCTCGT | |
| | GGCCGCCCTGGTCCTGGCTTTCTCCGGCATCATGATTGTGTACCGCCGGAAGCACCAG | |
| | GAGCTGCAAGCCATGCAGATGGAGCTGCAGAGCCCTGAGTACAAGCTGAGCAAGCTCC | |
| | GCACCTCGACCATCATGACCGACTACAACCCCAACTACTGCTTTGCTGGCAAGACCTC | |
| | CTCCATCAGTGACCTGAAGGAGGTGCCGCGGAAAAACATCACCCTCATTCGGGGTCTG | |
| | GGCCATGGCGCCTTTGGGGAGGTGTATGAAGGCCAGGTGTCCGGAATGCCCAACGACC | |
| | CAAGCCCCCTGCAAGTGGCTGTGAAGACGCTGCCTGAAGTGTGCTCTGAACAGGACGA | |
| | ACTGGATTTCCTCATGGAAGCCCTGATCATCAGCAAATTCAACCACCAGAACATTGTT | |
| | CGCTGCATTGGGGTGAGCCTGCAATCCCTGCCCCGGTTCATCCTGCTGGAGCTCATGG | |
| | CGGGGGGAGACCTCAAGTCCTTCCTCCGAGAGACCCGCCCTCGCCCGAGCCAGCCCTC | |
| | CTCCCTGGCCATGCTGGACCTTCTGCACGTGGCTCGGGACATTGCCTGTGGCTGTCAG | |
| | TATTTGGAGGAAAACCACTTCATCCACCGAGACATTGCTGCCAGAAACTGCCTCTTGA | |
| | CCTGTCCAGGCCCTGGAAGAGTGGCCAAGATTGGAGACTTCGGGATGGCCCGAGACAT | |
| | CTACAGGGCGAGCTACTATAGAAAGGGAGGCTGTGCCATGCTGCCAGTTAAGTGGATG | |
| | CCCCCAGAGGCCTTCATGGAAGGAATATTCACTTCTAAAACAGACACATGGTCCTTTG | |
| | GAGTGCTGCTATGGGAAATCTTTTCTCTTGGATATATGCCATACCCCAGCAAAAGCAA | |
| | CCAGGAAGTTCTGGAGTTTGTCACCAGTGGAGGCCGGATGGACCCACCCAAGAACTGC | |
| | CCTGGGCCTGTATACCGGATAATGACTCAGTGCTGGCAACATCAGCCTGAAGACAGGC | |
| | CCAACTTTGCCATCATTTTGGAGAGGATTGAATACTGCACCCAGGACCCGGATGTAAT | |
| | CAACACCGCTTTGCCGATAGAATATGGTCCACTTGTGGAAGAGGAAGAGAAAGTGCCT | |
| | GTGAGGCCCAAGGACCCTGAGGGGGTTCCTCCTCTCCTGGTCTCTCAACAGGCAAAAC | |
| | GGGAGGAGGAGCGCAGCCCAGCTGCCCCACCACCTCTGCCTACCACCTCCTCTGGCAA | |
| | GGCTGCAAAGAAACCCACAGCTGCAGAGATCTCTGTTCGAGTCCCTAGAGGGCCGGCC | |
| | GTGGAAGGGGGACACGTGAATATGGCATTCTCTCAGTCCAACCCTCCTTCGGAGTTGC | |
| | ACAAGGTCCACGGATCCAGAAACAAGCCCACCAGCTTGTGGAACCCAACGTACGGCTC | |
| | CTGGTTTACAGAGAAACCCACCAAAAAGAATAATCCTATAGCAAAGAAGGAGCCACAC | |
| | GACAGGGGTAACCTGGGGCTGGAGGGAAGCTGTACTGTCCCACCTAACGTTGCAACTG | |
| | GGAGACTTCCGGGGGCCTCACTGCTCCTAGAGCCCTCTTCGCTGACTGCCAATATGAA | |
| | GGAGGTACCTCTGTTCAGGCTACGTCACTTCCCTTGTGGGAATGTCAATTACGGCTAC | |
| | CAGCAACAGGGCTTGCCCTTAGAAGCCGCTACTGCCCCTGGAGCTGGTCATTACGAGG | |
| | ATACCATTCTGAAAAGCAAGAATAGCATGAACCAGCCTGGGCCCTGA | |
| ALK-UTRN | ATGGCCAAGTATGGAGAACATGAAGCCAGTCCTGACAATGGGCAGAACGAATTCAGTG | 30 |
| | ATATCATTAAGTCCAGATCTGATGAACACAATGACGTACAGAAGAAAACCTTTACCAA | |
| | ATGGATAAATGCTCGATTTTCAAAGAGTGGGAAACCACCCATCAATGATATGTTCACA | |
| | GACCTCAAAGATGGAAGGAAGCTATTGGATCTTCTAGAAGGCCTCACAGGAACATCAC | |
| | TGCCAAAGGAACGTGGTTCCACAAGGGTACATGCCTTAAATAACGTCAACAGAGTGCT | |
| | GCAGGTTTTACATCAGAACAATGTGGAATTAGTGAATATAGGGGGAACTGACATTGTG | |
| | GATGGAAATCACAAACTGACTTTGGGGTTACTTTGGAGCATCATTTTGCACTGGCAGG | |
| | TGAAAGATGTCATGAAGGATGTCATGTCGGACCTGCAGCAGACGAACAGTGAGAAGAT | |
| | CCTGCTCAGCTGGGTGCGTCAGACCACCAGGCCCTACAGCCAAGTCAACGTCCTCAAC | |
| | TTCACCACCAGCTGGACAGATGGACTCGCCTTTAATGCTGTCCTCCACCGACATAAAC | |
| | CTGATCTCTTCAGCTGGGATAAAGTTGTCAAAATGTCACCAATTGAGAGACTTGAACA | |
| | TGCCTTCAGCAAGGCTCAAACTTATTTGGGAATTGAAAAGCTGTTAGATCCTGAAGAT | |
| | GTTGCCGTTCAGCTTCCTGACAAGAAATCCATAATTATGTATTTAACATCTTTGTTTG | |
| | AGGTGCTACCTCAGCAAGTCACCATAGACGCCATCCGTGAGGTAGAGACACTCCCAAG | |
| | GAAATATAAAAAAGAATGTGAAGAAGAGGCAATTAATATACAGAGTACAGCGCCTGAG | |
| | GAGGAGCATGAGAGTCCCCGAGCTGAAACTCCCAGCACTGTCACTGAGGTTGACATGG | |
| | ATCTGGACAGCTATCAGATTGCGTTGGAGGAAGTGCTGACCTGGTTGCTTTCTGCTGA | |
| | GGACACTTTCCAGGAGCAGGATGATATTTCTGATGATGTTGAAGAAGTCAAAGACCAG | |
| | TTTGCAACCCATGAAGCTTTTATGATGGAACTGACTGCACACCAGAGCAGTGTGGGCA | |
| | GCGTCCTGCAGGCAGGCAACCAACTGATAACACAAGGAACTCTGTCAGACGAAGAAGA | |
| | ATTTGAGATTCAGGAACAGATGACCCTGCTGAATGCTAGATGGGAGGCTCTTAGGGTG | |
| | GAGAGTATGGACAGACAGTCCCGGCTGCACGATGTGCTGATGGAACTGCAGAAGAAGC | |
| | AACTGCAGCAGCTCTCCGCCTGGTTAACACTCACAGAGGAGCGCATTCAGAAGATGGA | |
| | AACTTGCCCCCTGGATGATGATGTAAAATCTCTACAAAAGCTGCTAGAAGAACATAAA | |
| | AGTTTGCAAAGTGATCTTGAGGCTGAACAGGTGAAAGTAAATTCACTAACTCACATGG | |
| | TGGTCATTGTTGATGAAAACAGTGGTGAGAGTGCTACAGCTATCCTAGAAGACCAGTT | |
| | ACAGAAACTTGGTGAGCGCTGGACAGCAGTATGCCGTTGGACTGAAGAACGCTGGAAT | |
| | AGGTTACAAGAAATCAATATATTGTGGCAGGAATTATTGGAAGAACAGTGCTTGTTGA | |
| | AAGCTTGGTTAACCGAAAAAGAAGAGGCTTTAAATAAAGTCCAGACAAGCAACTTCAA | |
| | AGACCAAAAGGAACTAAGTGTCAGTGTTCGACGTCTGGCTATTTTGAAGGAAGACATG | |
| | GAAATGAAGCGTCAAACATTGGATCAGCTGAGTGAGATTGGCCAGGATGTGGGACAAT | |
| | TACTTGATAATTCCAAGGCATCTAAGAAGATCAACAGTGACTCAGAGGAACTGACTCA | |
| | AAGATGGGATTCTTTGGTTCAGAGACTAGAAGATTCCTCCAACCAGGTGACTCAGGCT | |
| | GTAGCAAAGCTGGGGATGTCTCAGATTCCTCAGAAGGACCTTTTGGAGACTGTTCGTG | |
| | TAAGAGAACAAGCAATTACAAAAAAATCTAAGCAGGAACTGCCTCCTCCTCCTCCCCC | |
| | AAAGAAGAGACAGATCCATGTGGATATTGAAGCTAAGAAAAAGTTTGATGCTATAAGT | |
| | GCAGAGCTGTTGAACTGGATTTTGAAATGGAAAACTGCCATTCAGACCACAGAGATAA | |
| | AAGAGTATATGAAGATGCAAGACACTTCCGAAATGAAAAAGAAGTTGAAGGCATTAGA | |
| | AAAAGAACAGAGAGAAAGAATCCCCAGAGCAGATGAATTAAACCAAACTGGACAAATC | |
| | CTTGTGGAGCAAATGGGAAAAGAAGGCCTTCCTACTGAAGAAATAAAAAATGTTCTGG | |
| | AGAAGGTTTCATCAGAATGGAAGAATGTATCTCAACATTTGGAAGATCTAGAAAGAAA | |
| | GATTCAGCTACAGGAAGATATAAATGCTTATTTCAAGCAGCTTGATGAGCTTGAAAAG | |
| | GTCATCAAGACAAAGGAGGAGTGGGTAAAACACACTTCCATTTCTGAATCTTCCCGGC | |
| | AGTCCTTGCCAAGCTTGAAGGATTCCTGTCAGCGGGAATTGACAAATCTTCTTGGCCT | |
| | TCACCCCAAAATTGAAATGGCTCGTGCAAGCTGCTCGGCCCTGATGTCTCAGCCTTCT | |
| | GCCCCAGATTTTGTCCAGCGGGGCTTCGATAGCTTTCTGGGCCGCTACCAAGCTGTAC | |
| | AAGAGGCTGTAGAGGATCGTCAACAACATCTAGAGAATGAACTGAAGGGCCAACCTGG | |
| | ACATGCATATCTGGAAACATTGAAAACACTGAAAGATGTGCTAAATGATTCAGAAAAT | |
| | AAGGCCCAGGTGTCTCTGAATGTCCTTAATGATCTTGCCAAGGTGGAGAAGGCCCTGC | |
| | AAGAAAAAAAGACCCTTGATGAAATCCTTGAGAATCAGAAACCTGCATTACATAAACT | |
| | TGCAGAAGAAACAAAGGCTCTGGAGAAAAATGTTCATCCTGATGTAGAAAAATTATAT | |
| | AAGCAAGAATTTGATGATGTGCAAGGAAAGTGGAACAAGCTAAAGGTCTTGGTTTCCA | |
| | AAGATCTACATTTGCTTGAGGAAATTGCTCTCACACTCAGAGCTTTTGAGGCCGATTC | |
| | AACAGTCATTGAGAAGTGGATGGATGGCGTGAAAGACTTCTTAATGAAACAGCAGGCT | |
| | GCCCAAGGAGACGACGCAGGTCTACAGAGGCAGTTAGACCAGTGCTCTGCATTTGTTA | |
| | ATGAAATAGAAACAATTGAATCATCTCTGAAAAACATGAAGGAAATAGAGACTAATCT | |
| | TCGAAGTGGTCCAGTTGCTGGAATAAAAACTTGGGTGCAGACAAGACTAGGTGACTAC | |
| | CAAACTCAACTGGAGAAACTTAGCAAGGAGATCGCTACTCAAAAAAGTAGGTTGTCTG | |
| | AAAGTCAAGAAAAAGCTGCGAACCTGAAGAAAGACTTGGCAGAGATGCAGGAATGGAT | |
| | GACCCAGGCCGAGGAAGAATATTTGGAGCGGGATTTTGAGTACAAGTCACCAGAAGAG | |
| | CTTGAGAGTGCTGTGGAAGAGATGAAGAGGGCAAAAGAGGATGTGTTGCAGAAGGAGG | |
| | TGAGAGTGAAGATTCTCAAGGACAACATCAAGTTATTAGCTGCCAAGGTGCCCTCTGG | |
| | TGGCCAGGAGTTGACGTCTGAGCTGAATGTTGTGCTGGAGAATTACCAACTTCTTTGT | |
| | AATAGAATTCGAGGAAAGTGCCACACGCTAGAGGAGGTCTGGTCTTGTTGGATTGAAC | |
| | TGCTTCACTATTTGGATCTTGAAACTACCTGGTTAAACACTTTGGAAGAGCGGATGAA | |
| | GAGCACAGAGGTCCTGCCTGAGAAGACGGATGCTGTCAACGAAGCCCTGGAGTCTCTG | |
| | GAATCTGTTCTGCGCCACCCGGCAGATAATCGCACCCAGATTCGAGAGCTTGGCCAGA | |
| | CTCTGATTGATGGGGGGATCCTGGATGATATAATCAGTGAGAAACTGGAGGCTTTCAA | |
| | CAGCCGATATGAAGATCTAAGTCACCTGGCAGAGAGCAAGCAGATTTCTTTGGAAAAG | |
| | CAACTCCAGGTGCTGCGGGAAACTGACCAGATGCTTCAAGTCTTGCAAGAGAGCTTGG | |
| | GGGAGCTGGACAAACAGCTCACCACATACCTGACTGACAGGATAGATGCTTTCCAAGT | |
| | TCCACAGGAAGCTCAGAAAATCCAAGCAGAGATCTCAGCCCATGAGCTAACCCTAGAG | |
| | GAGTTGAGAAGAAATATGCGTTCTCAGCCCCTGACCTCCCCAGAGAGTAGGACTGCCA | |
| | GAGGAGGAAGTCAGATGGATGTGCTACAGAGGAAACTCCGAGAGGTGTCCACAAAGTT | |
| | CCAGCTTTTCCAGAAGCCAGCTAACTTCGAGCAGCGCATGCTGGACTGCAAGCGTGTG | |
| | CTGGATGGCGTGAAAGCAGAACTTCACGTTCTGGATGTGAAGGACGTAGACCCTGACG | |
| | TCATACAGACGCACCTGGACAAGTGTATGAAACTGTATAAAACTTTGAGTGAAGTCAA | |
| | ACTTGAAGTGGAAACTGTGATTAAAACAGGAAGACATATTGTCCAGAAACAGCAAACG | |
| | GACAACCCAAAAGGGATGGATGAGCAGCTGACTTCCCTGAAGGTTCTTTACAATGACC | |
| | TGGGCGCACAGGTGACAGAAGGAAAACAGGATCTGGAAAGAGCATCACAGTTGGCCCG | |
| | GAAAATGAAGAAAGAGGCTGCTTCTCTCTCTGAATGGCTTTCTGCTACTGAAACTGAA | |
| | TTGGTACAGAAGTCCACTTCAGAAGGTCTGCTTGGTGACTTGGATACAGAAATTTCCT | |
| | GGGCTAAAAATGTTCTGAAGGATCTGGAAAAGAGAAAAGCTGATTTAAATACCATCAC | |
| | AGAGAGTAGTGCTGCCCTGCAAAACTTGATTGAGGGCAGTGAGCCTATTTTAGAAGAG | |
| | AGGCTCTGCGTCCTTAACGCTGGGTGGAGCCGAGTTCGTACCTGGACTGAAGATTGGT | |
| | GCAATACCTTGATGAACCATCAGAACCAGCTAGAAATATTTGATGGGAACGTGGCTCA | |
| | CATAAGTACCTGGCTTTATCAAGCTGAAGCTCTATTGGATGAAATTGAAAAGAAACCA | |
| | ACAAGTAAACAGGAAGAAATTGTGAAGCGTTTAGTATCTGAGCTGGATGATGCCAACC | |
| | TCCAGGTTGAAAATGTCCGCGATCAAGCCCTTATTTTGATGAATGCCCGTGGAAGCTC | |
| | AAGCAGGGAGCTTGTAGAACCAAAGTTAGCTGAGCTGAATAGGAACTTTGAAAAGGTG | |
| | TCTCAACATATCAAAAGTGCCAAATTGCTAATTGCTCAGGAACCATTATACCAATGTT | |
| | TGGTCACCACTGAAACATTTGAAACTGGTGTGCCTTTCTCTGACTTGGAAAAATTAGA | |
| | AAATGACATAGAAAATATGTTAAAATTTGTGGAAAAACACTTGGAATCCAGTGATGAA | |
| | GATGAAAAGATGGATGAGGAGAGTGCCCAGATTGAGGAAGTTCTACAAAGAGGAGAAG | |
| | AAATGTTACATCAACCTATGGAAGATAATAAAAAAGAAAAGATCCGTTTGCAATTATT | |
| | ACTTTTGCATACTAGATACAACAAAATTAAGGCAATCCCTATTCAACAGAGGAAAATG | |
| | GGTCAACTTGCTTCTGGAATTAGATCATCACTTCTTCCTACAGATTATCTGGTTGAAA | |
| | TTAACAAAATTTTACTTTGCATGGATGATGTTGAATTATCGCTTAATGTTCCAGAGCT | |
| | CAACACTGCTATTTACGAAGACTTCTCTTTTCAGGAAGACTCTCTGAAGAATATCAAA | |
| | GACCAACTGGACAAACTTGGAGAGCAGATTGCAGTCATTCATGAAAAACAGCCAGATG | |
| | TCATCCTTGAAGCCTCTGGACCTGAAGCCATTCAGATCAGAGATACACTTACTCAGCT | |
| | GAATGCAAAATGGGACAGAATTAATAGAATGTACAGTGATCGGAAAGGTTGTTTTGAC | |
| | AGGGCAATGGAAGAATGGAGACAGTTCCATTGTGACCTTAATGACCTCACACAGTGGA | |
| | TAACAGAGGCTGAAGAATTACTGGTTGATACCTGTGCTCCAGGTGGCAGCCTGGACTT | |
| | AGAGAAAGCCAGGATACATCAGCAGGAACTTGAGGTGGGCATCAGCAGCCACCAGCCC | |
| | AGTTTTGCAGCACTAAACCGAACTGGGGATGGGATTGTGCAGAAACTCTCCCAGGCAG | |
| | ATGGAAGCTTCTTGAAAGAAAAACTGGCAGGTTTAAACCAACGCTGGGATGCAATTGT | |
| | TGCAGAAGTGAAGGATAGGCAGCCAAGGCTAAAAGGAGAAAGTAAGCAGGTGATGAAG | |
| | TACAGGCATCAGCTAGATGAGATTATCTGTTGGTTAACAAAGGCTGAGCATGCTATGC | |
| | AAAAGAGATCAACCACCGAATTGGGAGAAAACCTGCAAGAATTAAGAGACTTAACTCA | |
| | AGAAATGGAAGTACATGCTGAAAAACTCAAATGGCTGAATAGAACTGAATTGGAGATG | |
| | CTTTCAGATAAAAGTCTGAGTTTACCTGAAAGGGATAAAATTTCAGAAAGCTTAAGGA | |
| | CTGTAAATATGACATGGAATAAGATTTGCAGAGAGGTGCCTACCACCCTGAAGGAATG | |
| | CATCCAGGAGCCCAGTTCTGTTTCACAGACAAGGATTGCTGCTCATCCTAATGTCCAA | |
| | AAGGTGGTGCTAGTATCATCTGCGTCAGATATTCCTGTTCAGTCTCATCGTACTTCGG | |
| | AAATTTCAATTCCTGCTGATCTTGATAAAACTATAACAGAACTAGCCGACTGGCTGGT | |
| | ATTAATCGACCAGATGCTGAAGTCCAACATTGTCACTGTTGGGGATGTAGAAGAGATC | |
| | AATAAGACCGTTTCCCGAATGAAAATTACAAAGGCTGACTTAGAACAGCGCCATCCTC | |
| | AGCTGGATTATGTTTTTACATTGGCACAGAATTTGAAAAATAAAGCTTCCAGTTCAGA | |
| | TATGAGAACAGCAATTACAGAAAAATTGGAAAGGGTCAAGAACCAGTGGGATGGCACC | |
| | CAGCATGGCGTTGAGCTAAGACAGCAGCAGCTTGAGGACATGATTATTGACAGTCTTC | |
| | AGTGGGATGACCATAGGGAGGAGACTGAAGAACTGATGAGAAAATATGAGGCTCGACT | |
| | CTATATTCTTCAGCAAGCCCGACGGGATCCACTCACCAAACAAATTTCTGATAACCAA | |
| | ATACTGCTTCAAGAACTGGGTCCTGGAGATGGTATCGTCATGGCGTTCGATAACGTCC | |
| | TGCAGAAACTCCTGGAGGAATATGGGAGTGATGACACAAGGAATGTGAAAGAAACCAC | |
| | AGAGTACTTAAAAACATCATGGATCAATCTCAAACAAAGTATTGCTGACAGACAGAAC | |
| | GCCTTGGAGGCTGAGTGGAGGACGGTGCAGGCCTCTCGCAGAGATCTGGAAAACTTCC | |
| | TGAAGTGGATCCAAGAAGCAGAGACCACAGTGAATGTGCTTGTGGATGCCTCTCATCG | |
| | GGAGAATGCTCTTCAGGATAGTATCTTGGCCAGGGAACTCAAACAGCAGATGCAGGAC | |
| | ATCCAGGCAGAAATTGATGCCCACAATGACATATTTAAAAGCATTGACGGAAACAGGC | |
| | AGAAGATGGTAAAAGCTTTGGGAAATTCTGAAGAGGCTACTATGCTTCAACATCGACT | |
| | GGATGATATGAACCAAAGATGGAATGACTTAAAAGCAAAATCTGCTAGCATCAGGGCC | |
| | CATTTGGAGGCCAGCGCTGAGAAGTGGAACAGGTTGCTGATGTCCTTAGAAGAACTGA | |
| | TCAAATGGCTGAATATGAAAGATGAAGAGCTTAAGAAACAAATGCCTATTGGAGGAGA | |
| | TGTTCCAGCCTTACAGCTCCAGTATGACCATTGTAAGGCCCTGAGACGGGAGTTAAAG | |
| | GAGAAAGAATATTCTGTCCTGAATGCTGTCGACCAGGCCCGAGTTTTCTTGGCTGATC | |
| | AGCCAATTGAGGCCCCTGAAGAGCCAAGAAGAAACCTACAATCAAAAACAGAATTAAC | |
| | TCCTGAGGAGAGAGCCCAAAAGATTGCCAAAGCCATGCGCAAACAGTCTTCTGAAGTC | |
| | AAAGAAAAATGGGAAAGTCTAAATGCTGTAACTAGCAATTGGCAAAAGCAAGTGGACA | |
| | AGGCATTGGAGAAACTCAGAGACCTGCAGGGAGCTATGGATGACCTGGACGCTGACAT | |
| | GAAGGAGGCAGAGTCCGTGCGGAATGGCTGGAAGCCCGTGGGAGACTTACTCATTGAC | |
| | TCGCTGCAGGATCACATTGAAAAAATCATGGCATTTAGAGAAGAAATTGCACCAATCA | |
| | ACTTTAAAGTTAAAACGGTGAATGATTTATCCAGTCAGCTGTCTCCACTTGACCTGCA | |
| | TCCCTCTCTAAAGATGTCTCGCCAGCTAGATGACCTTAATATGCGATGGAAACTTTTA | |
| | CAGGTTTCTGTGGATGATCGCCTTAAACAGCTTCAGGAAGCCCACAGAGATTTTGGAC | |
| | CATCCTCTCAGCATTTTCTCTCTACGTCAGTCCAGCTGCCGTGGCAAAGATCCATTTC | |
| | ACATAATAAAGTGCCCTATTACATCAACCATCAAACACAGACCACCTGTTGGGACCAT | |
| | CCTAAAATGACCGAACTCTTTCAATCCCTTGCTGACCTGAATAATGTACGTTTTTCTG | |
| | CCTACCGTACAGCAATCAAAATCCGAAGACTACAAAAAGCACTATGTTTGGATCTCTT | |
| | AGAGTTGAGTACAACAAATGAAATTTTCAAACAGCACAAGTTGAACCAAAATGACCAG | |
| | CTCCTCAGTGTTCCAGATGTCATCAACTGTCTGACAACAACTTATGATGGACTTGAGC | |
| | AAATGCATAAGGACCTGGTCAACGTTCCACTCTGTGTTGATATGTGTCTCAATTGGTT | |
| | GCTCAATGTCTATGACACGGGTCGAACTGGAAAAATTAGAGTGCAGAGTCTGAAGATT | |
| | GGATTAATGTCTCTCTCCAAAGGTCTCTTGGAAGAAAAATACAGATATCTCTTTAAGG | |
| | AAGTTGCAGGGCCAACAGAAATGTGTGACCAGAGGCAGCTGGGCCTGTTACTTCATGA | |
| | TGCCATCCAGATCCCCCGGCAGCTAGGTGAAGTAGCAGCTTTTGGAGGCAGTAATATT | |
| | GAGCCTAGTGTTCGCAGCTGCTTCCAACAGAATAACAATAAACCAGAAATAAGTGTGA | |
| | AAGAGTTTATAGATTGGATGCATTTGGAACCACAGTCCATGGTTTGGCTCCCAGTTTT | |
| | ACATCGAGTGGCAGCAGCGGAGACTGCAAAACATCAGGCCAAATGCAACATCTGTAAA | |
| | GAATGTCCAATTGTCGGGTTCAGGTATAGAAGCCTTAAGCATTTTAACTATGATGTCT | |
| | GCCAGAGTTGTTTCTTTTCGGGTCGAACAGCAAAAGGTCACAAATTACATTACCCAAT | |
| | GGTGGAATATTGTATACCTACAACATCTGGGGAAGATGTACGAGACTTCACAAAGGTA | |
| | CTTAAGAACAAGTTCAGGTCGAAGAAGTACTTTGCCAAACACCCTCGACTTGGTTACC | |
| | TGCCTGTCCAGACAGTTCTTGAAGGTGACAACTTAGAGACTCCTATCACACTCATCAG | |
| | TATGTGGCCAGAGCACTATGACCCCTCACAATCTCCTCAACTGTTTCATGATGACACC | |
| | CATTCAAGAATAGAACAATATGCCACACGACTGGCCCAGATGGAAAGGACTAATGGGT | |
| | CTTTTCTCACTGATAGCAGCTCCACCACAGGAAGTGTGGAAGACGAGCACGCCCTCAT | |
| | CCAGCAGTATTGCCAAACACTCGGAGGAGAGTCCCCAGTGAGCCAGCCGCAGAGCCCA | |
| | GCTCAGATCCTGAAGTCAGTAGAGAGGGAAGAACGTGGAGAACTGGAGAGGATCATTG | |
| | CTGACCTGGAGGAAGAACAAAGAAATCTACAGGTGGAGTATGAGCAGCTGAAGGACCA | |
| | GCACCTCCGAAGGGGGCTCCCTGTCGGTTCACCGCCAGAGTCGATTATATCTCCCCAT | |
| | CACACGTCTGAGGATTCAGAACTTATAGCAGAAGCAAAACTCCTCAGGCAGCACAAAG | |
| | GTCGGCTGGAGGCTAGGATGCAGATTTTAGAAGATCACAATAAACAGCTGGAGTCTCA | |
| | GCTCCACCGCCTCCGACAGCTGCTGGAGCAGCCTGAATCTGATTCCCGAATCAATGGT | |
| | GTTTCCCCATGGGCTTCTCCTCAGCATTCTGCACTGAGCTACTCGCTTGATCCAGATG | |
| | CCTCCGGCCCACAGTTCCACCAGGCAGTGTCACCCACCCCGGAGCCACACCTGCCACT | |
| | CTCGCTGATCCTCTCTGTGGTGACCTCTGCCCTCGTGGCCGCCCTGGTCCTGGCTTTC | |
| | TCCGGCATCATGATTGTGTACCGCCGGAAGCACCAGGAGCTGCAAGCCATGCAGATGG | |
| | AGCTGCAGAGCCCTGAGTACAAGCTGAGCAAGCTCCGCACCTCGACCATCATGACCGA | |
| | CTACAACCCCAACTACTGCTTTGCTGGCAAGACCTCCTCCATCAGTGACCTGAAGGAG | |
| | GTGCCGCGGAAAAACATCACCCTCATTCGGGGTCTGGGCCATGGCGCCTTTGGGGAGG | |
| | TGTATGAAGGCCAGGTGTCCGGAATGCCCAACGACCCAAGCCCCCTGCAAGTGGCTGT | |
| | GAAGACGCTGCCTGAAGTGTGCTCTGAACAGGACGAACTGGATTTCCTCATGGAAGCC | |
| | CTGATCATCAGCAAATTCAACCACCAGAACATTGTTCGCTGCATTGGGGTGAGCCTGC | |
| | AATCCCTGCCCCGGTTCATCCTGCTGGAGCTCATGGCGGGGGGAGACCTCAAGTCCTT | |
| | CCTCCGAGAGACCCGCCCTCGCCCGAGCCAGCCCTCCTCCCTGGCCATGCTGGACCTT | |
| | CTGCACGTGGCTCGGGACATTGCCTGTGGCTGTCAGTATTTGGAGGAAAACCACTTCA | |
| | TCCACCGAGACATTGCTGCCAGAAACTGCCTCTTGACCTGTCCAGGCCCTGGAAGAGT | |
| | GGCCAAGATTGGAGACTTCGGGATGGCCCGAGACATCTACAGGGCGAGCTACTATAGA | |
| | AAGGGAGGCTGTGCCATGCTGCCAGTTAAGTGGATGCCCCCAGAGGCCTTCATGGAAG | |
| | GAATATTCACTTCTAAAACAGACACATGGTCCTTTGGAGTGCTGCTATGGGAAATCTT | |
| | TTCTCTTGGATATATGCCATACCCCAGCAAAAGCAACCAGGAAGTTCTGGAGTTTGTC | |
| | ACCAGTGGAGGCCGGATGGACCCACCCAAGAACTGCCCTGGGCCTGTATACCGGATAA | |
| | TGACTCAGTGCTGGCAACATCAGCCTGAAGACAGGCCCAACTTTGCCATCATTTTGGA | |
| | GAGGATTGAATACTGCACCCAGGACCCGGATGTAATCAACACCGCTTTGCCGATAGAA | |
| | TATGGTCCACTTGTGGAAGAGGAAGAGAAAGTGCCTGTGAGGCCCAAGGACCCTGAGG | |
| | GGGTTCCTCCTCTCCTGGTCTCTCAACAGGCAAAACGGGAGGAGGAGCGCAGCCCAGC | |
| | TGCCCCACCACCTCTGCCTACCACCTCCTCTGGCAAGGCTGCAAAGAAACCCACAGCT | |
| | GCAGAGATCTCTGTTCGAGTCCCTAGAGGGCCGGCCGTGGAAGGGGGACACGTGAATA | |
| | TGGCATTCTCTCAGTCCAACCCTCCTTCGGAGTTGCACAAGGTCCACGGATCCAGAAA | |
| | CAAGCCCACCAGCTTGTGGAACCCAACGTACGGCTCCTGGTTTACAGAGAAACCCACC | |
| | AAAAAGAATAATCCTATAGCAAAGAAGGAGCCACACGACAGGGGTAACCTGGGGCTGG | |
| | AGGGAAGCTGTACTGTCCCACCTAACGTTGCAACTGGGAGACTTCCGGGGGCCTCACT | |
| | GCTCCTAGAGCCCTCTTCGCTGACTGCCAATATGAAGGAGGTACCTCTGTTCAGGCTA | |
| | CGTCACTTCCCTTGTGGGAATGTCAATTACGGCTACCAGCAACAGGGCTTGCCCTTAG | |
| | AAGCCGCTACTGCCCCTGGAGCTGGTCATTACGAGGATACCATTCTGAAAAGCAAGAA | |
| | TAGCATGAACCAGCCTGGGCCCTGA | |
| ALK- | ATGGTGGACTACCACGCGGCGAACCAGTCGTACCAGTACGGCCCCAGCAGCGCGGGCA | 31 |
| ACTN4 | ATGGCGCTGGCGGCGGGGGCAGCATGGGCGACTACATGGCCCAGGAGGACGACTGGGA | |
| | CCGGGACCTGCTGCTGGACCCGGCCTGGGAGAAGCAGCAGCGCAAGACCTTCACGGCA | |
| | TGGTGCAACTCCCACCTGCGGAAGGCAGGCACACAGATCGAGAACATTGATGAGGACT | |
| | TCCGAGACGGGCTCAAGCTCATGCTGCTCCTGGAGGTCATATCAGGGGAGCGGTTACC | |
| | TAAGCCGGAGCGGGGGAAGATGAGAGTGCACAAAATCAACAATGTGAACAAAGCGCTG | |
| | GACTTTATTGCCAGCAAAGGCGTCAAGCTGGTCTCCATCGGGGCAGAAGAGATTGTGG | |
| | ACGGCAACGCAAAGATGACCCTGGGAATGATCTGGACCATCATCCTTAGGTTCGCCAT | |
| | CCAGGACATCTCCGTGGAAGAGACCTCGGCCAAGGAAGGGCTCCTTCTCTGGTGCCAG | |
| | AGAAAGACAGCCCCGTATAAGAACGTCAATGTGCAGAACTTCCACATCAGCTGGAAGG | |
| | ATGGTCTTGCCTTCAATGCCCTGATCCACCGGCACAGACCAGAGCTGATTGAGTATGA | |
| | CAAGCTGAGGAAGGACGACCCTGTCACCAACCTGAACAATGCCTTCGAAGTGGCTGAG | |
| | AAATACCTCGACATCCCCAAGATGCTGGATGCAGAGGTGTACCGCCGGAAGCACCAGG | |
| | AGCTGCAAGCCATGCAGATGGAGCTGCAGAGCCCTGAGTACAAGCTGAGCAAGCTCCG | |
| | CACCTCGACCATCATGACCGACTACAACCCCAACTACTGCTTTGCTGGCAAGACCTCC | |
| | TCCATCAGTGACCTGAAGGAGGTGCCGCGGAAAAACATCACCCTCATTCGGGGTCTGG | |
| | GCCATGGCGCCTTTGGGGAGGTGTATGAAGGCCAGGTGTCCGGAATGCCCAACGACCC | |
| | AAGCCCCCTGCAAGTGGCTGTGAAGACGCTGCCTGAAGTGTGCTCTGAACAGGACGAA | |
| | CTGGATTTCCTCATGGAAGCCCTGATCATCAGCAAATTCAACCACCAGAACATTGTTC | |
| | GCTGCATTGGGGTGAGCCTGCAATCCCTGCCCCGGTTCATCCTGCTGGAGCTCATGGC | |
| | GGGGGGAGACCTCAAGTCCTTCCTCCGAGAGACCCGCCCTCGCCCGAGCCAGCCCTCC | |
| | TCCCTGGCCATGCTGGACCTTCTGCACGTGGCTCGGGACATTGCCTGTGGCTGTCAGT | |
| | ATTTGGAGGAAAACCACTTCATCCACCGAGACATTGCTGCCAGAAACTGCCTCTTGAC | |
| | CTGTCCAGGCCCTGGAAGAGTGGCCAAGATTGGAGACTTCGGGATGGCCCGAGACATC | |
| | TACAGGGCGAGCTACTATAGAAAGGGAGGCTGTGCCATGCTGCCAGTTAAGTGGATGC | |
| | CCCCAGAGGCCTTCATGGAAGGAATATTCACTTCTAAAACAGACACATGGTCCTTTGG | |
| | AGTGCTGCTATGGGAAATCTTTTCTCTTGGATATATGCCATACCCCAGCAAAAGCAAC | |
| | CAGGAAGTTCTGGAGTTTGTCACCAGTGGAGGCCGGATGGACCCACCCAAGAACTGCC | |
| | CTGGGCCTGTATACCGGATAATGACTCAGTGCTGGCAACATCAGCCTGAAGACAGGCC | |
| | CAACTTTGCCATCATTTTGGAGAGGATTGAATACTGCACCCAGGACCCGGATGTAATC | |
| | AACACCGCTTTGCCGATAGAATATGGTCCACTTGTGGAAGAGGAAGAGAAAGTGCCTG | |
| | TGAGGCCCAAGGACCCTGAGGGGGTTCCTCCTCTCCTGGTCTCTCAACAGGCAAAACG | |
| | GGAGGAGGAGCGCAGCCCAGCTGCCCCACCACCTCTGCCTACCACCTCCTCTGGCAAG | |
| | GCTGCAAAGAAACCCACAGCTGCAGAGATCTCTGTTCGAGTCCCTAGAGGGCCGGCCG | |
| | TGGAAGGGGGACACGTGAATATGGCATTCTCTCAGTCCAACCCTCCTTCGGAGTTGCA | |
| | CAAGGTCCACGGATCCAGAAACAAGCCCACCAGCTTGTGGAACCCAACGTACGGCTCC | |
| | TGGTTTACAGAGAAACCCACCAAAAAGAATAATCCTATAGCAAAGAAGGAGCCACACG | |
| | ACAGGGGTAACCTGGGGCTGGAGGGAAGCTGTACTGTCCCACCTAACGTTGCAACTGG | |
| | GAGACTTCCGGGGGCCTCACTGCTCCTAGAGCCCTCTTCGCTGACTGCCAATATGAAG | |
| | GAGGTACCTCTGTTCAGGCTACGTCACTTCCCTTGTGGGAATGTCAATTACGGCTACC | |
| | AGCAACAGGGCTTGCCCTTAGAAGCCGCTACTGCCCCTGGAGCTGGTCATTACGAGGA | |
| | TACCATTCTGAAAAGCAAGAATAGCATGAACCAGCCTGGGCCCTGA | |
| ALK- | ATGGGACGGCCGGAGTCTGCGGGCGGAGGGAGTCGCGGGCCTTTTGAGGGAGGAGGCA | 32 |
| CPSF7 | GAGCGCGCCGGGCCGGTGGCATCTTCCTTACTTTGTCCATCCTCCGGACTCGCGATCT | |
| | TCCTTCCGGAGCCATGTCAGAAGGAGTGGACTTGATTGATATATATGCTGACGAGGAG | |
| | TTCAACCAGGACCCAGAGTTCAACAATACAGATCAGATTGACCTGTATGATGATGTGC | |
| | TGACAGCCACCTCACAGCCCTCAGATGACAGAAGCAGCAGCACTGAACCACCTCCTCC | |
| | TGTTCGCCAGGAGCCATCTCCCAAGCCCAACAACAAGACCCCTGCAATTCTGTATACC | |
| | TACAGTGGCCTGCGTAATAGACGAGCTGCCGTTTATGTGGGCAGCTTCTCCTGGTGGA | |
| | CCACAGACCAGCAGCTGATCCAGGTTATTCGCTCTATAGGAGTCTATGATGTGGTGGA | |
| | GTTGAAATTTGCAGAGAATCGAGCAAATGGCCAGTCCAAAGGGTATGCTGAGGTGGTG | |
| | GTAGCCTCTGAAAACTCTGTCCACAAATTGTTGGAACTCCTACCAGGGAAAGTTCTTA | |
| | ATGGAGAAAAAGTGGACGTGAGGCCGGCCACCCGGCAGAACCTGTCACAGTTTGAGGC | |
| | ACAGGCTCGGAAACGTGAGTGTGTCCGAGTCCCAAGAGGGGTGTACCGCCGGAAGCAC | |
| | CAGGAGCTGCAAGCCATGCAGATGGAGCTGCAGAGCCCTGAGTACAAGCTGAGCAAGC | |
| | TCCGCACCTCGACCATCATGACCGACTACAACCCCAACTACTGCTTTGCTGGCAAGAC | |
| | CTCCTCCATCAGTGACCTGAAGGAGGTGCCGCGGAAAAACATCACCCTCATTCGGGGT | |
| | CTGGGCCATGGCGCCTTTGGGGAGGTGTATGAAGGCCAGGTGTCCGGAATGCCCAACG | |
| | ACCCAAGCCCCCTGCAAGTGGCTGTGAAGACGCTGCCTGAAGTGTGCTCTGAACAGGA | |
| | CGAACTGGATTTCCTCATGGAAGCCCTGATCATCAGCAAATTCAACCACCAGAACATT | |
| | GTTCGCTGCATTGGGGTGAGCCTGCAATCCCTGCCCCGGTTCATCCTGCTGGAGCTCA | |
| | TGGCGGGGGGAGACCTCAAGTCCTTCCTCCGAGAGACCCGCCCTCGCCCGAGCCAGCC | |
| | CTCCTCCCTGGCCATGCTGGACCTTCTGCACGTGGCTCGGGACATTGCCTGTGGCTGT | |
| | CAGTATTTGGAGGAAAACCACTTCATCCACCGAGACATTGCTGCCAGAAACTGCCTCT | |
| | TGACCTGTCCAGGCCCTGGAAGAGTGGCCAAGATTGGAGACTTCGGGATGGCCCGAGA | |
| | CATCTACAGGGCGAGCTACTATAGAAAGGGAGGCTGTGCCATGCTGCCAGTTAAGTGG | |
| | ATGCCCCCAGAGGCCTTCATGGAAGGAATATTCACTTCTAAAACAGACACATGGTCCT | |
| | TTGGAGTGCTGCTATGGGAAATCTTTTCTCTTGGATATATGCCATACCCCAGCAAAAG | |
| | CAACCAGGAAGTTCTGGAGTTTGTCACCAGTGGAGGCCGGATGGACCCACCCAAGAAC | |
| | TGCCCTGGGCCTGTATACCGGATAATGACTCAGTGCTGGCAACATCAGCCTGAAGACA | |
| | GGCCCAACTTTGCCATCATTTTGGAGAGGATTGAATACTGCACCCAGGACCCGGATGT | |
| | AATCAACACCGCTTTGCCGATAGAATATGGTCCACTTGTGGAAGAGGAAGAGAAAGTG | |
| | CCTGTGAGGCCCAAGGACCCTGAGGGGGTTCCTCCTCTCCTGGTCTCTCAACAGGCAA | |
| | AACGGGAGGAGGAGCGCAGCCCAGCTGCCCCACCACCTCTGCCTACCACCTCCTCTGG | |
| | CAAGGCTGCAAAGAAACCCACAGCTGCAGAGATCTCTGTTCGAGTCCCTAGAGGGCCG | |
| | GCCGTGGAAGGGGGACACGTGAATATGGCATTCTCTCAGTCCAACCCTCCTTCGGAGT | |
| | TGCACAAGGTCCACGGATCCAGAAACAAGCCCACCAGCTTGTGGAACCCAACGTACGG | |
| | CTCCTGGTTTACAGAGAAACCCACCAAAAAGAATAATCCTATAGCAAAGAAGGAGCCA | |
| | CACGACAGGGGTAACCTGGGGCTGGAGGGAAGCTGTACTGTCCCACCTAACGTTGCAA | |
| | CTGGGAGACTTCCGGGGGCCTCACTGCTCCTAGAGCCCTCTTCGCTGACTGCCAATAT | |
| | GAAGGAGGTACCTCTGTTCAGGCTACGTCACTTCCCTTGTGGGAATGTCAATTACGGC | |
| | TACCAGCAACAGGGCTTGCCCTTAGAAGCCGCTACTGCCCCTGGAGCTGGTCATTACG | |
| | AGGATACCATTCTGAAAAGCAAGAATAGCATGAACCAGCCTGGGCCCTGA | |
| ALK- | ATGGCGGCCGCCAAGGTGGCTTTAACCAAGAGAGCAGATCCAGCTGAGCTTAGAACAA | 33 |
| SLC25A13 | TATTTTTGAAGTATGCAAGCATTGAGAAAAACGGTGAATTTTTCATGTCCCCCAATGA | |
| | CTTTGTCACTCGATACTTGAACATTTTTGGAGAAAGCCAGCCTAATCCAAAGACTGTG | |
| | GAACTTTTAAGTGGAGTGGTGGATCAGACCAAAGATGGATTAATATCTTTTCAAGAAT | |
| | TTGTTGCCTTTGAATCTGTCCTGTGTGCCCCTGATGCTTTGTTTATGGTAGCCTTTCA | |
| | GCTGTTTGACAAAGCTGGCAAAGGAGAAGTAACTTTTGAGGATGTTAAGCAAGTTTTT | |
| | GGACAGACCACAATTCATCAACATATTCCATTTAACTGGGATTCAGAATTTGTGCAAC | |
| | TACATTTTGGAAAAGAAAGAAAAAGACACCTGACATATGCGGAATTTACTCAGTTTTT | |
| | ATTGGAAATACAACTGGAGCACGCAAAGCAAGCCTTTGTGCAACGGGACAATGCTAGG | |
| | ACTGGGAGAGTCACAGCCATCGACTTCCGAGACATCATGGTCACCATCCGCCCCCATG | |
| | TCTTGACTCCTTTTGTAGAAGAATGTCTAGTAGCTGCTGCTGGAGGTACCACATCCCA | |
| | TCAAGTTAGTTTCTCCTATTTTAATGGATTTAATTCGCTCCTTAACAACATGGAACTC | |
| | ATTAGAAAGATCTATAGCACTCTGGCTGGCACCAGGAAAGATGTTGAAGTGACTAAGG | |
| | AGGAGTTTGTTCTGGCAGCTCAGAAATTTGGTCAGGTTACACCCATGGAAGTTGACAT | |
| | CTTGTTTCAGTTAGCAGATTTATATGAGCCAAGGGGACGTATGACCTTAGCAGACATT | |
| | GAACGGATTGCTCCTCTGGAAGAGGGAACTCTGCCCTTTAACTTGGCTGAGGCCCAGA | |
| | GGCAGAAGGCCTCAGGTGATTCAGCTCGACCAGTTCTTCTACAAGTTGCAGAGTCGGC | |
| | CTACAGGTTTGGTCTGGGTTCTGTTGCTGGAGTGTACCGCCGGAAGCACCAGGAGCTG | |
| | CAAGCCATGCAGATGGAGCTGCAGAGCCCTGAGTACAAGCTGAGCAAGCTCCGCACCT | |
| | CGACCATCATGACCGACTACAACCCCAACTACTGCTTTGCTGGCAAGACCTCCTCCAT | |
| | CAGTGACCTGAAGGAGGTGCCGCGGAAAAACATCACCCTCATTCGGGGTCTGGGCCAT | |
| | GGCGCCTTTGGGGAGGTGTATGAAGGCCAGGTGTCCGGAATGCCCAACGACCCAAGCC | |
| | CCCTGCAAGTGGCTGTGAAGACGCTGCCTGAAGTGTGCTCTGAACAGGACGAACTGGA | |
| | TTTCCTCATGGAAGCCCTGATCATCAGCAAATTCAACCACCAGAACATTGTTCGCTGC | |
| | ATTGGGGTGAGCCTGCAATCCCTGCCCCGGTTCATCCTGCTGGAGCTCATGGCGGGGG | |
| | GAGACCTCAAGTCCTTCCTCCGAGAGACCCGCCCTCGCCCGAGCCAGCCCTCCTCCCT | |
| | GGCCATGCTGGACCTTCTGCACGTGGCTCGGGACATTGCCTGTGGCTGTCAGTATTTG | |
| | GAGGAAAACCACTTCATCCACCGAGACATTGCTGCCAGAAACTGCCTCTTGACCTGTC | |
| | CAGGCCCTGGAAGAGTGGCCAAGATTGGAGACTTCGGGATGGCCCGAGACATCTACAG | |
| | GGCGAGCTACTATAGAAAGGGAGGCTGTGCCATGCTGCCAGTTAAGTGGATGCCCCCA | |
| | GAGGCCTTCATGGAAGGAATATTCACTTCTAAAACAGACACATGGTCCTTTGGAGTGC | |
| | TGCTATGGGAAATCTTTTCTCTTGGATATATGCCATACCCCAGCAAAAGCAACCAGGA | |
| | AGTTCTGGAGTTTGTCACCAGTGGAGGCCGGATGGACCCACCCAAGAACTGCCCTGGG | |
| | CCTGTATACCGGATAATGACTCAGTGCTGGCAACATCAGCCTGAAGACAGGCCCAACT | |
| | TTGCCATCATTTTGGAGAGGATTGAATACTGCACCCAGGACCCGGATGTAATCAACAC | |
| | CGCTTTGCCGATAGAATATGGTCCACTTGTGGAAGAGGAAGAGAAAGTGCCTGTGAGG | |
| | CCCAAGGACCCTGAGGGGGTTCCTCCTCTCCTGGTCTCTCAACAGGCAAAACGGGAGG | |
| | AGGAGCGCAGCCCAGCTGCCCCACCACCTCTGCCTACCACCTCCTCTGGCAAGGCTGC | |
| | AAAGAAACCCACAGCTGCAGAGATCTCTGTTCGAGTCCCTAGAGGGCCGGCCGTGGAA | |
| | GGGGGACACGTGAATATGGCATTCTCTCAGTCCAACCCTCCTTCGGAGTTGCACAAGG | |
| | TCCACGGATCCAGAAACAAGCCCACCAGCTTGTGGAACCCAACGTACGGCTCCTGGTT | |
| | TACAGAGAAACCCACCAAAAAGAATAATCCTATAGCAAAGAAGGAGCCACACGACAGG | |
| | GGTAACCTGGGGCTGGAGGGAAGCTGTACTGTCCCACCTAACGTTGCAACTGGGAGAC | |
| | TTCCGGGGGCCTCACTGCTCCTAGAGCCCTCTTCGCTGACTGCCAATATGAAGGAGGT | |
| | ACCTCTGTTCAGGCTACGTCACTTCCCTTGTGGGAATGTCAATTACGGCTACCAGCAA | |
| | CAGGGCTTGCCCTTAGAAGCCGCTACTGCCCCTGGAGCTGGTCATTACGAGGATACCA | |
| | TTCTGAAAAGCAAGAATAGCATGAACCAGCCTGGGCCCTGA | |
| ALK- | ATGGACGACTCAGAGGTGGAGTCGACCGCCAGCATCTTGGCCTCTGTGAAGGAACAAG | 34 |
| CTNND1 | AGGCCCAGTTTGAGAAGCTGACCCGGGCGCTGGAGGAGGAACGGCGCCACGTCTCGGC | |
| | GCAGCTGGAACGCGTCCGGGTCTCACCACAAGATGCCAACCCACTCATGGCCAACGGC | |
| | ACACTCACCCGCCGGCATCAGAACGGCCGGTTTGTGGGCGATGCTGACCTTGAAAGAC | |
| | AGAAATTTTCAGATTTGAAACTCAACGGACCCCAGGATCACAGTCACCTTCTATATAG | |
| | CACCATCCCCAGGATGCAGGAGCCGGGGCAGATTGTGGAGACCTACACGGAGGAGGAT | |
| | CCTGAGGGAGCCATGTCTGTAGTCTCTGTGGAGACCTCAGATGATGGGACCACTCGGC | |
| | GCACAGAGACCACGGTCAAGAAAGTAGTGAAGACTGTGACAACACGGACAGTACAGCC | |
| | AGTCGCTATGGGACCAGACGGGTTGCCTGTGGATGCTTCATCAGTTTCTAACAACTAT | |
| | ATCCAGACTTTGGGTCGTGATTTCCGCAAGAATGGCAATGGGGGACCTGGTCCCTATG | |
| | TGGGGCAAGCTGGCACTGCTACCCTTCCTAGGAACTTCCACTACCCTCCTGATGGTTA | |
| | TAGTCGCCACTATGAAGATGGTTATCCAGGTGGCAGTGATAACTATGGCAGTCTGTCC | |
| | CGGGTGACCCGCATTGAGGAGCGGTATAGGCCCAGCATGGAAGGCTACCGGGCACCTA | |
| | GTAGACAGGATGTGTATGGGCCCCAACCCCAGGTTCGGGTAGGTGGGAGCAGCGTGGA | |
| | TCTGCATCGCTTTCATCCAGAGCCTTATGGGCTAGAGGATGACCAGCGTAGTATGGGC | |
| | TATGATGACCTGGATTATGGTATGATGTCTGATTATGGCACTGCCCGTCGGACTGGGA | |
| | CACCCTCTGACCCTCGTCGGCGCCTCAGGAGCTATGAAGACATGATTGGTGAGGAGGT | |
| | GCCATCGGATCAATACTACTGGGCTCCTTTGGCCCAGCATGAGCGAGGAAGTTTAGCA | |
| | AGCTTGGATAGCCTGCGCAAAGGAGGGCCTCCACCTCCTAATTGGAGACAGCCAGAGC | |
| | TGCCAGAGGTGATCGCCATGCTTGGATTCCGCTTGGATGCTGTCAAGTCCAATGCAGC | |
| | TGCATACCTGCAACACTTATGCTACCGCAATGACAAGGTGAAGACTGACGTGCGGAAG | |
| | CTCAAGGGCATCCCAGTACTGGTGGGATTGTTAGACCATCCCAAAAAGGAAGTGCACC | |
| | TTGGAGCCTGTGGAGCTCTCAAGAATATCTCTTTTGGACGTGACCAGGATAACAAGAT | |
| | TGCCATAAAAAACTGTGATGGTGTGCCTGCCCTTGTGCGATTGCTTCGAAAGGCTCGT | |
| | GATATGGACCTTACTGAAGTTATTACCGGAACCCTGTGGAATCTTTCATCCCATGACT | |
| | CAATCAAAATGGAGATTGTGGACCATGCACTGCATGCCTTGACAGATGAAGTGATCAT | |
| | TCCTCATTCTGGTTGGGAGCGGGAACCTAATGAAGACTGTAAGCCACGCCACATTGAG | |
| | TGGGAATCGGTGCTCACCAACACAGCTGGCTGCCTTAGGAATGTAAGCTCAGAGAGGA | |
| | GTGAAGCTCGCCGGAAACTTCGGGAATGTGATGGTTTAGTTGATGCCCTCATTTTCAT | |
| | TGTTCAGGCTGAGATTGGGCAGAAGGATTCAGACAGCAAGCTTGTAGAGAACTGTGTT | |
| | TGCCTTCTTCGGAACTTATCATATCAAGTTCACCGGGAGATCCCACAGGCAGAGCGTT | |
| | ACCAAGAGGCAGCTCCCAATGTTGCCAACAATACTGGGCCACATGCTGCCAGTTGCTT | |
| | TGGGGCCAAGAAGGGCAAAGGGAAAAAACCTATAGAGGATCCAGCAAACGATACAGTG | |
| | GATTTCCCTAAAAGAACGAGTCCAGCTCGAGGCTATGAGCTCTTATTTCAGCCAGAGG | |
| | TGGTTCGGATATACATCTCACTTCTTAAGGAGAGCAAGACTCCTGCCATCCTAGAAGC | |
| | CTCAGCTGGAGCTATCCAGAACTTGTGTGCTGGGCGCTGGACGTATGGTCGATACATC | |
| | CGCTCTGCTCTGCGTCAAGAGAAGGCTCTTTCTGCCATAGCTGACCTCCTGACTAATG | |
| | AACATGAACGGGTGGTGAAAGCTGCATCTGGAGCACTGAGAAACCTGGCTGTGGATGC | |
| | TCGCAACAAAGAATTAATTGGTAAACATGCTATTCCTAACTTGGTAAAGAATCTGCCA | |
| | GGAGGACAGCAGAACTCCTCTTGGAATTTCTCTGAGGACACTGTCATCTCTATTTTGA | |
| | ACACTATCAACGAGGTTATCGCTGAGAACTTGGAGGCTGCCAAAAAGCTTCGAGAGAC | |
| | ACAGGGTATTGAGAAGCTGGTGTTGATCAACAAATCAGGGAACCGCTCAGAAAAAGAA | |
| | GTTCGAGCAGCAGCACTTGTATTACAGACAATCTGGGGATATAAGGAACTGCGGAAGC | |
| | CACTGGAAAAAGAAGGATGGAAGAAATCAGACTTTCAGGTGAATCTAAACAATGCTTC | |
| | CCGAAGCCAGAGCAGTCATTCATATGATGATAGTACTCTCCCTCTCATTGACCGGAAC | |
| | CAAAAATCAGTGTACCGCCGGAAGCACCAGGAGCTGCAAGCCATGCAGATGGAGCTGC | |
| | AGAGCCCTGAGTACAAGCTGAGCAAGCTCCGCACCTCGACCATCATGACCGACTACAA | |
| | CCCCAACTACTGCTTTGCTGGCAAGACCTCCTCCATCAGTGACCTGAAGGAGGTGCCG | |
| | CGGAAAAACATCACCCTCATTCGGGGTCTGGGCCATGGCGCCTTTGGGGAGGTGTATG | |
| | AAGGCCAGGTGTCCGGAATGCCCAACGACCCAAGCCCCCTGCAAGTGGCTGTGAAGAC | |
| | GCTGCCTGAAGTGTGCTCTGAACAGGACGAACTGGATTTCCTCATGGAAGCCCTGATC | |
| | ATCAGCAAATTCAACCACCAGAACATTGTTCGCTGCATTGGGGTGAGCCTGCAATCCC | |
| | TGCCCCGGTTCATCCTGCTGGAGCTCATGGCGGGGGGAGACCTCAAGTCCTTCCTCCG | |
| | AGAGACCCGCCCTCGCCCGAGCCAGCCCTCCTCCCTGGCCATGCTGGACCTTCTGCAC | |
| | GTGGCTCGGGACATTGCCTGTGGCTGTCAGTATTTGGAGGAAAACCACTTCATCCACC | |
| | GAGACATTGCTGCCAGAAACTGCCTCTTGACCTGTCCAGGCCCTGGAAGAGTGGCCAA | |
| | GATTGGAGACTTCGGGATGGCCCGAGACATCTACAGGGCGAGCTACTATAGAAAGGGA | |
| | GGCTGTGCCATGCTGCCAGTTAAGTGGATGCCCCCAGAGGCCTTCATGGAAGGAATAT | |
| | TCACTTCTAAAACAGACACATGGTCCTTTGGAGTGCTGCTATGGGAAATCTTTTCTCT | |
| | TGGATATATGCCATACCCCAGCAAAAGCAACCAGGAAGTTCTGGAGTTTGTCACCAGT | |
| | GGAGGCCGGATGGACCCACCCAAGAACTGCCCTGGGCCTGTATACCGGATAATGACTC | |
| | AGTGCTGGCAACATCAGCCTGAAGACAGGCCCAACTTTGCCATCATTTTGGAGAGGAT | |
| | TGAATACTGCACCCAGGACCCGGATGTAATCAACACCGCTTTGCCGATAGAATATGGT | |
| | CCACTTGTGGAAGAGGAAGAGAAAGTGCCTGTGAGGCCCAAGGACCCTGAGGGGGTTC | |
| | CTCCTCTCCTGGTCTCTCAACAGGCAAAACGGGAGGAGGAGCGCAGCCCAGCTGCCCC | |
| | ACCACCTCTGCCTACCACCTCCTCTGGCAAGGCTGCAAAGAAACCCACAGCTGCAGAG | |
| | ATCTCTGTTCGAGTCCCTAGAGGGCCGGCCGTGGAAGGGGGACACGTGAATATGGCAT | |
| | TCTCTCAGTCCAACCCTCCTTCGGAGTTGCACAAGGTCCACGGATCCAGAAACAAGCC | |
| | CACCAGCTTGTGGAACCCAACGTACGGCTCCTGGTTTACAGAGAAACCCACCAAAAAG | |
| | AATAATCCTATAGCAAAGAAGGAGCCACACGACAGGGGTAACCTGGGGCTGGAGGGAA | |
| | GCTGTACTGTCCCACCTAACGTTGCAACTGGGAGACTTCCGGGGGCCTCACTGCTCCT | |
| | AGAGCCCTCTTCGCTGACTGCCAATATGAAGGAGGTACCTCTGTTCAGGCTACGTCAC | |
| | TTCCCTTGTGGGAATGTCAATTACGGCTACCAGCAACAGGGCTTGCCCTTAGAAGCCG | |
| | CTACTGCCCCTGGAGCTGGTCATTACGAGGATACCATTCTGAAAAGCAAGAATAGCAT | |
| | GAACCAGCCTGGGCCCTGA | |
| ALK-KLC4 | ATGTCAGGCCTGGTGTTGGGGCAGCGGGATGAGCCTGCAGGCCACCGGCTCAGCCAAG | 35 |
| | AGGAGATCCTGGGGAGCACACGGCTGGTCAGCCAAGGGCTAGAGGCCCTACGCAGTGA | |
| | ACACCAGGCCGTGCTGCAAAGCCTGTCCCAGACCATTGAGTGTCTGCAGCAGGGAGGC | |
| | CATGAGGAAGGGCTGGTGCATGAGAAGGCCCGGCAGCTTCGCCGTTCTATGGAAAACA | |
| | TTGAGCTCGGGCTGAGTGAGGCCCAGGTGATGCTGGCTCTAGCCAGCCACCTGAGCAC | |
| | AGTGGAGTCGGAGAAACAGAAGCTGCGGGCTCAGGTGCGGCGGCTATGCCAGGAGAAC | |
| | CAGTGGCTGCGGGATGAGCTGGCTGGCACCCAGCAGCGGCTACAGCGCAGTGAACAGG | |
| | CTGTGGCTCAGCTGGAGGAGGAAAAGAAGCACCTGGAGTTCCTGGGGCAGCTGCGGCA | |
| | GTATGATGAGGATGGACATACCTCGGAGGAGAAAGAAGGCGATGCCACCAAGGATTCC | |
| | CTGGATGACCTCTTTCCTAATGAGGAGGAAGAGGACCCCAGCAATGGCTTGTCCCGTG | |
| | GTCAAGGTGCTACAGCAGCTCAGCAGGGTGGATATGAGATCCCAGCAAGGTTGCGGAC | |
| | GTTGCACAACCTGGTGATCCAGTACGCAGCCCAAGGTCGCTATGAGGTGGCCGTGCCA | |
| | CTCTGTAAGCAGGCACTAGAGGACCTGGAGCGCACATCAGGCCGTGGCCACCCTGATG | |
| | TCGCCACCATGCTCAACATCCTTGCTTTGGTGTATCGTGACCAGAATAAGTATAAGGA | |
| | AGCTGCCCACCTGCTGAATGATGCCCTTAGCATCCGGGAGAGCACCTTGGGACCTGAC | |
| | CATCCTGCTGTGGCTGCCACACTCAACAATTTGGCTGTGCTCTATGGCAAAAGGGGCA | |
| | AGTACAAGGAGGCAGAGCCTCTGTGCCAGCGGGCACTGGAGATTCGAGAAAAGGTCCT | |
| | GGGCACGAATCATCCAGATGTGGCAAAACAGCTGAACAACCTGGCCCTCTTGTGCCAA | |
| | AACCAGGGCAAGTATGAGGCCGTGGAACGCTACTACCAGCGAGCACTGGCCATCTACG | |
| | AGGGGCAGCTGGGGCCGGACAACCCTAATGTAGCCCGGACCAAGAACAACCTGGCTTC | |
| | CTGTTACCTGAAACAGGGCAAATATGCTGAGGCTGAGACACTATACAAAGAGATCCTG | |
| | ACCCGTGCCCATGTACAGGAGTTTGGGTCTGTGGATGTGTACCGCCGGAAGCACCAGG | |
| | AGCTGCAAGCCATGCAGATGGAGCTGCAGAGCCCTGAGTACAAGCTGAGCAAGCTCCG | |
| | CACCTCGACCATCATGACCGACTACAACCCCAACTACTGCTTTGCTGGCAAGACCTCC | |
| | TCCATCAGTGACCTGAAGGAGGTGCCGCGGAAAAACATCACCCTCATTCGGGGTCTGG | |
| | GCCATGGCGCCTTTGGGGAGGTGTATGAAGGCCAGGTGTCCGGAATGCCCAACGACCC | |
| | AAGCCCCCTGCAAGTGGCTGTGAAGACGCTGCCTGAAGTGTGCTCTGAACAGGACGAA | |
| | CTGGATTTCCTCATGGAAGCCCTGATCATCAGCAAATTCAACCACCAGAACATTGTTC | |
| | GCTGCATTGGGGTGAGCCTGCAATCCCTGCCCCGGTTCATCCTGCTGGAGCTCATGGC | |
| | GGGGGGAGACCTCAAGTCCTTCCTCCGAGAGACCCGCCCTCGCCCGAGCCAGCCCTCC | |
| | TCCCTGGCCATGCTGGACCTTCTGCACGTGGCTCGGGACATTGCCTGTGGCTGTCAGT | |
| | ATTTGGAGGAAAACCACTTCATCCACCGAGACATTGCTGCCAGAAACTGCCTCTTGAC | |
| | CTGTCCAGGCCCTGGAAGAGTGGCCAAGATTGGAGACTTCGGGATGGCCCGAGACATC | |
| | TACAGGGCGAGCTACTATAGAAAGGGAGGCTGTGCCATGCTGCCAGTTAAGTGGATGC | |
| | CCCCAGAGGCCTTCATGGAAGGAATATTCACTTCTAAAACAGACACATGGTCCTTTGG | |
| | AGTGCTGCTATGGGAAATCTTTTCTCTTGGATATATGCCATACCCCAGCAAAAGCAAC | |
| | CAGGAAGTTCTGGAGTTTGTCACCAGTGGAGGCCGGATGGACCCACCCAAGAACTGCC | |
| | CTGGGCCTGTATACCGGATAATGACTCAGTGCTGGCAACATCAGCCTGAAGACAGGCC | |
| | CAACTTTGCCATCATTTTGGAGAGGATTGAATACTGCACCCAGGACCCGGATGTAATC | |
| | AACACCGCTTTGCCGATAGAATATGGTCCACTTGTGGAAGAGGAAGAGAAAGTGCCTG | |
| | TGAGGCCCAAGGACCCTGAGGGGGTTCCTCCTCTCCTGGTCTCTCAACAGGCAAAACG | |
| | GGAGGAGGAGCGCAGCCCAGCTGCCCCACCACCTCTGCCTACCACCTCCTCTGGCAAG | |
| | GCTGCAAAGAAACCCACAGCTGCAGAGATCTCTGTTCGAGTCCCTAGAGGGCCGGCCG | |
| | TGGAAGGGGGACACGTGAATATGGCATTCTCTCAGTCCAACCCTCCTTCGGAGTTGCA | |
| | CAAGGTCCACGGATCCAGAAACAAGCCCACCAGCTTGTGGAACCCAACGTACGGCTCC | |
| | TGGTTTACAGAGAAACCCACCAAAAAGAATAATCCTATAGCAAAGAAGGAGCCACACG | |
| | ACAGGGGTAACCTGGGGCTGGAGGGAAGCTGTACTGTCCCACCTAACGTTGCAACTGG | |
| | GAGACTTCCGGGGGCCTCACTGCTCCTAGAGCCCTCTTCGCTGACTGCCAATATGAAG | |
| | GAGGTACCTCTGTTCAGGCTACGTCACTTCCCTTGTGGGAATGTCAATTACGGCTACC | |
| | AGCAACAGGGCTTGCCCTTAGAAGCCGCTACTGCCCCTGGAGCTGGTCATTACGAGGA | |
| | TACCATTCTGAAAAGCAAGAATAGCATGAACCAGCCTGGGCCCTGA | |
| ALK- | ATGAGTAACAGCCACCCTCTTCGCCCCTTTACTGCAGTGGGGGAAATTGATCATGTGC | 36 |
| BTBD9 | ACATTTTGTCTGAACATATTGGTGCCTTGTTGATTGGGGAAGAATATGGCGACGTCAC | |
| | ATTCGTGGTGGAAAAGAAACGTTTTCCTGCCCACAGGGTAATTTTAGCAGCCAGGTGC | |
| | CAATATTTTCGAGCATTATTATATGGTGGAATGCGAGAGTCTCAGCCTGAAGCAGAAA | |
| | TTCCTCTCCAAGACACCACTGCAGAAGCATTCACAATGCTACTCAAATATATCTACAC | |
| | TGGGCGGGCAACGCTGACAGATGAGAAGGAGGAGGTGCTGCTGGACTTTTTGAGCCTG | |
| | GCTCATAAATATGGATTTCCAGAGCTAGAGGATTCTACCTCTGAGTATCTCTGCACCA | |
| | TACTTAACATTCAGAATGTCTGCATGACTTTTGATGTTGCCAGTCTCTACTCACTTCC | |
| | CAAGTTAACTTGTATGTGCTGCATGTTTATGGATAGGAATGCTCAGGAAGTCCTCTCA | |
| | AGTGAAGGTTTCCTCTCCCTTTCTAAGACAGCACTTTTAAACATCGTGTTAAGAGACT | |
| | CATTTGCAGCTCCCGAAAAAGATATTTTCCTAGCCTTATTAAACTGGTGTAAGCACAA | |
| | TTCAAAGGAGAATCATGCTGAAATCATGCAGGCTGTGCGTTTACCTCTCATGAGCCTC | |
| | ACAGAGCTTCTGAATGTTGTGAGGCCTTCAGGACTGCTGTCTCCTGATGCCATCCTGG | |
| | ATGCCATTAAAGTGCGATCTGAGAGCCGGGATATGGACCTCAATTATAGAGGCATGCT | |
| | CATGTACCGCCGGAAGCACCAGGAGCTGCAAGCCATGCAGATGGAGCTGCAGAGCCCT | |
| | GAGTACAAGCTGAGCAAGCTCCGCACCTCGACCATCATGACCGACTACAACCCCAACT | |
| | ACTGCTTTGCTGGCAAGACCTCCTCCATCAGTGACCTGAAGGAGGTGCCGCGGAAAAA | |
| | CATCACCCTCATTCGGGGTCTGGGCCATGGCGCCTTTGGGGAGGTGTATGAAGGCCAG | |
| | GTGTCCGGAATGCCCAACGACCCAAGCCCCCTGCAAGTGGCTGTGAAGACGCTGCCTG | |
| | AAGTGTGCTCTGAACAGGACGAACTGGATTTCCTCATGGAAGCCCTGATCATCAGCAA | |
| | ATTCAACCACCAGAACATTGTTCGCTGCATTGGGGTGAGCCTGCAATCCCTGCCCCGG | |
| | TTCATCCTGCTGGAGCTCATGGCGGGGGGAGACCTCAAGTCCTTCCTCCGAGAGACCC | |
| | GCCCTCGCCCGAGCCAGCCCTCCTCCCTGGCCATGCTGGACCTTCTGCACGTGGCTCG | |
| | GGACATTGCCTGTGGCTGTCAGTATTTGGAGGAAAACCACTTCATCCACCGAGACATT | |
| | GCTGCCAGAAACTGCCTCTTGACCTGTCCAGGCCCTGGAAGAGTGGCCAAGATTGGAG | |
| | ACTTCGGGATGGCCCGAGACATCTACAGGGCGAGCTACTATAGAAAGGGAGGCTGTGC | |
| | CATGCTGCCAGTTAAGTGGATGCCCCCAGAGGCCTTCATGGAAGGAATATTCACTTCT | |
| | AAAACAGACACATGGTCCTTTGGAGTGCTGCTATGGGAAATCTTTTCTCTTGGATATA | |
| | TGCCATACCCCAGCAAAAGCAACCAGGAAGTTCTGGAGTTTGTCACCAGTGGAGGCCG | |
| | GATGGACCCACCCAAGAACTGCCCTGGGCCTGTATACCGGATAATGACTCAGTGCTGG | |
| | CAACATCAGCCTGAAGACAGGCCCAACTTTGCCATCATTTTGGAGAGGATTGAATACT | |
| | GCACCCAGGACCCGGATGTAATCAACACCGCTTTGCCGATAGAATATGGTCCACTTGT | |
| | GGAAGAGGAAGAGAAAGTGCCTGTGAGGCCCAAGGACCCTGAGGGGGTTCCTCCTCTC | |
| | CTGGTCTCTCAACAGGCAAAACGGGAGGAGGAGCGCAGCCCAGCTGCCCCACCACCTC | |
| | TGCCTACCACCTCCTCTGGCAAGGCTGCAAAGAAACCCACAGCTGCAGAGATCTCTGT | |
| | TCGAGTCCCTAGAGGGCCGGCCGTGGAAGGGGGACACGTGAATATGGCATTCTCTCAG | |
| | TCCAACCCTCCTTCGGAGTTGCACAAGGTCCACGGATCCAGAAACAAGCCCACCAGCT | |
| | TGTGGAACCCAACGTACGGCTCCTGGTTTACAGAGAAACCCACCAAAAAGAATAATCC | |
| | TATAGCAAAGAAGGAGCCACACGACAGGGGTAACCTGGGGCTGGAGGGAAGCTGTACT | |
| | GTCCCACCTAACGTTGCAACTGGGAGACTTCCGGGGGCCTCACTGCTCCTAGAGCCCT | |
| | CTTCGCTGACTGCCAATATGAAGGAGGTACCTCTGTTCAGGCTACGTCACTTCCCTTG | |
| | TGGGAATGTCAATTACGGCTACCAGCAACAGGGCTTGCCCTTAGAAGCCGCTACTGCC | |
| | CCTGGAGCTGGTCATTACGAGGATACCATTCTGAAAAGCAAGAATAGCATGAACCAGC | |
| | CTGGGCCCTGA | |
| ALK-CPQ | ATGAAATTCCTTATCTTCGCATTTTTCGGTGGTGTTCACCTTTTATCCCTGTGCTCTG | 38 |
| | GGAAAGCTATATGCAAGAATGGCATCTCTAAGAGGACTTTTGAAGAAATAAAAGAAGA | |
| | AATAGCCAGCTGTGGAGATGTTGCTAAAGCAATCATCAACCTAGCTGTTTATGGTAAA | |
| | GCCCAGAACAGATCCTATGAGCGATTGGCACTTCTGGTTGATACTGTTGGACCCAGAC | |
| | TGAGTGGCTCCAAGAACCTAGAAAAAGCCATCCAAATTATGTACCAAAACCTGCAGCA | |
| | AGATGGGCTGGAGAAAGTTCACCTGGAGCCAGTGAGAATACCCCACTGGGAGAGGGGA | |
| | GAAGAATCAGCTGTGATGCTGGAGCCAAGAATTCATAAGATAGCCATCCTGGGTCTTG | |
| | GCAGCAGCATTGGGACTCCTCCAGAAGGCATTACAGCAGAAGTTCTGGTGGTGACCTC | |
| | TTTCGATGAACTGCAGAGAAGGGCCTCAGAAGCAAGAGGGAAGATTGTTGTTTATAAC | |
| | CAACCTTACATCAACTACTCAAGGACGGTGCAATACCGAACGCAGGGGGCGGTGGAAG | |
| | CTGCCAAGGTGGGGGCTTTGGCATCTCTCATTCGATCCGTGGCCTCCTTCTCCATCTA | |
| | CAGTCCTCACACAGGTATTCAGGAATACCAGGATGGCGTGCCCAAGATTCCAACAGCC | |
| | TGTATTACGGTGGAAGATGCAGAAATGATGTCAAGAATGGCTTCTCATGGGATCAAAA | |
| | TTGTCATTCAGCTAAAGATGGGGGCAAAGACCTACCCAGATACTGATTCCTTCAACAC | |
| | TGTAGCAGAGATCACTGGGAGCAAATATCCAGAACAGGTTGTACTGGTCAGTGGACAT | |
| | CTGGACAGCTGGGATGTTGGGCAGGGTGCCATGGATGATGGCGGTGGAGCCTTTATAT | |
| | CATGGGAAGCACTCTCACTTATTAAAGATCTTGGGCTGCGTCCAAAGAGGACTCTGCG | |
| | GCTGGTGCTCTGGACTGCAGAAGAACAAGGTGGAGTTGGTGCCTTCCAGTATTATCAG | |
| | TTACACAAGGTAAATATTTCCAACTACAGTCTGGTGATGGAGTCTGACGCAGGAACCT | |
| | TCTTACCCACTGGGCTGCAATTCACTGGCAGTGAAAAGGCCAGGGCCATCATGGAGGA | |
| | GGTTATGAGCCTGCTGCAGCCCCTCAATATCACTCAGGTCCTGAGCCATGGAGAAGGG | |
| | ACAGACATCAACTTTTGGATCCAAGCTGGAGTGCCTGTGTACCGCCGGAAGCACCAGG | |
| | AGCTGCAAGCCATGCAGATGGAGCTGCAGAGCCCTGAGTACAAGCTGAGCAAGCTCCG | |
| | CACCTCGACCATCATGACCGACTACAACCCCAACTACTGCTTTGCTGGCAAGACCTCC | |
| | TCCATCAGTGACCTGAAGGAGGTGCCGCGGAAAAACATCACCCTCATTCGGGGTCTGG | |
| | GCCATGGCGCCTTTGGGGAGGTGTATGAAGGCCAGGTGTCCGGAATGCCCAACGACCC | |
| | AAGCCCCCTGCAAGTGGCTGTGAAGACGCTGCCTGAAGTGTGCTCTGAACAGGACGAA | |
| | CTGGATTTCCTCATGGAAGCCCTGATCATCAGCAAATTCAACCACCAGAACATTGTTC | |
| | GCTGCATTGGGGTGAGCCTGCAATCCCTGCCCCGGTTCATCCTGCTGGAGCTCATGGC | |
| | GGGGGGAGACCTCAAGTCCTTCCTCCGAGAGACCCGCCCTCGCCCGAGCCAGCCCTCC | |
| | TCCCTGGCCATGCTGGACCTTCTGCACGTGGCTCGGGACATTGCCTGTGGCTGTCAGT | |
| | ATTTGGAGGAAAACCACTTCATCCACCGAGACATTGCTGCCAGAAACTGCCTCTTGAC | |
| | CTGTCCAGGCCCTGGAAGAGTGGCCAAGATTGGAGACTTCGGGATGGCCCGAGACATC | |
| | TACAGGGCGAGCTACTATAGAAAGGGAGGCTGTGCCATGCTGCCAGTTAAGTGGATGC | |
| | CCCCAGAGGCCTTCATGGAAGGAATATTCACTTCTAAAACAGACACATGGTCCTTTGG | |
| | AGTGCTGCTATGGGAAATCTTTTCTCTTGGATATATGCCATACCCCAGCAAAAGCAAC | |
| | CAGGAAGTTCTGGAGTTTGTCACCAGTGGAGGCCGGATGGACCCACCCAAGAACTGCC | |
| | CTGGGCCTGTATACCGGATAATGACTCAGTGCTGGCAACATCAGCCTGAAGACAGGCC | |
| | CAACTTTGCCATCATTTTGGAGAGGATTGAATACTGCACCCAGGACCCGGATGTAATC | |
| | AACACCGCTTTGCCGATAGAATATGGTCCACTTGTGGAAGAGGAAGAGAAAGTGCCTG | |
| | TGAGGCCCAAGGACCCTGAGGGGGTTCCTCCTCTCCTGGTCTCTCAACAGGCAAAACG | |
| | GGAGGAGGAGCGCAGCCCAGCTGCCCCACCACCTCTGCCTACCACCTCCTCTGGCAAG | |
| | GCTGCAAAGAAACCCACAGCTGCAGAGATCTCTGTTCGAGTCCCTAGAGGGCCGGCCG | |
| | TGGAAGGGGGACACGTGAATATGGCATTCTCTCAGTCCAACCCTCCTTCGGAGTTGCA | |
| | CAAGGTCCACGGATCCAGAAACAAGCCCACCAGCTTGTGGAACCCAACGTACGGCTCC | |
| | TGGTTTACAGAGAAACCCACCAAAAAGAATAATCCTATAGCAAAGAAGGAGCCACACG | |
| | ACAGGGGTAACCTGGGGCTGGAGGGAAGCTGTACTGTCCCACCTAACGTTGCAACTGG | |
| | GAGACTTCCGGGGGCCTCACTGCTCCTAGAGCCCTCTTCGCTGACTGCCAATATGAAG | |
| | GAGGTACCTCTGTTCAGGCTACGTCACTTCCCTTGTGGGAATGTCAATTACGGCTACC | |
| | AGCAACAGGGCTTGCCCTTAGAAGCCGCTACTGCCCCTGGAGCTGGTCATTACGAGGA | |
| | TACCATTCTGAAAAGCAAGAATAGCATGAACCAGCCTGGGCCCTGA | |
| ALK-KIF5C | ATGGCGGATCCAGCCGAATGCAGCATCAAAGTGATGTGCCGGTTCCGGCCCCTCAACG | 40 |
| | AAGCGGAGATCCTCCGCGGGGACAAATTCATCCCCAAATTTAAAGGCGATGAGACCGT | |
| | GGTGATCGGGCAAGGGAAGCCATATGTCTTCGACAGAGTGCTACCTCCCAACACGACC | |
| | CAAGAGCAGGTTTACAATGCATGTGCGAAGCAAATTGTCAAAGATGTCCTTGAAGGTT | |
| | ATAACGGGACGATTTTTGCGTATGGGCAGACTTCATCAGGAAAAACCCACACCATGGA | |
| | GGGGAAGCTGCATGACCCCCAGCTCATGGGGATCATCCCACGAATTGCCCATGATATC | |
| | TTTGACCATATCTACTCCATGGATGAGAACCTGGAGTTTCACATAAAGGTTTCCTATT | |
| | TTGAGATCTACTTGGACAAAATAAGGGACTTACTTGATGTATCCAAGACCAACTTGGC | |
| | TGTTCATGAAGATAAAAACAGAGTCCCGTATGTAAAGGGGTGCACTGAGCGGTTTGTG | |
| | TCGAGCCCTGAGGAAGTCATGGATGTAATAGATGAAGGCAAAGCAAACCGACACGTGG | |
| | CTGTGACAAACATGAATGAACACAGCTCTAGAAGTCACAGTATCTTCCTGATAAATAT | |
| | TAAACAAGAGAATGTAGAGACTGAAAAAAAACTCAGTGGGAAACTTTATTTGGTTGAT | |
| | TTGGCTGGGAGCGAAAAGGTCAGCAAAACTGGTGCCGAGGGAGCTGTTCTTGACGAAG | |
| | CTAAAAATATCAATAAGTCTTTGTCTGCTCTTGGAAATGTGATCTCTGCTTTGGCAGA | |
| | AGGGACAAAAACACATGTGCCATACCGGGACAGCAAGATGACTCGGATTCTTCAGGAC | |
| | TCTTTGGGTGGGAACTGCAGAACCACCATCGTCATTTGCTGTTCTCCTTCTGTCTTCA | |
| | ATGAGGCTGAGACCAAGTCCACACTGATGTTCGGACAGAGAGCTAAGACCATCAAGAA | |
| | TACAGTCTCTGTGAACCTAGAACTGACAGCAGAAGAATGGAAGAAGAAATATGAAAAA | |
| | GAGAAAGAGAAAAACAAGACTTTGAAGAATGTTATCCAGCATCTGGAGATGGAGCTAA | |
| | ACAGGTGGAGGAATGTGTACCGCCGGAAGCACCAGGAGCTGCAAGCCATGCAGATGGA | |
| | GCTGCAGAGCCCTGAGTACAAGCTGAGCAAGCTCCGCACCTCGACCATCATGACCGAC | |
| | TACAACCCCAACTACTGCTTTGCTGGCAAGACCTCCTCCATCAGTGACCTGAAGGAGG | |
| | TGCCGCGGAAAAACATCACCCTCATTCGGGGTCTGGGCCATGGCGCCTTTGGGGAGGT | |
| | GTATGAAGGCCAGGTGTCCGGAATGCCCAACGACCCAAGCCCCCTGCAAGTGGCTGTG | |
| | AAGACGCTGCCTGAAGTGTGCTCTGAACAGGACGAACTGGATTTCCTCATGGAAGCCC | |
| | TGATCATCAGCAAATTCAACCACCAGAACATTGTTCGCTGCATTGGGGTGAGCCTGCA | |
| | ATCCCTGCCCCGGTTCATCCTGCTGGAGCTCATGGCGGGGGGAGACCTCAAGTCCTTC | |
| | CTCCGAGAGACCCGCCCTCGCCCGAGCCAGCCCTCCTCCCTGGCCATGCTGGACCTTC | |
| | TGCACGTGGCTCGGGACATTGCCTGTGGCTGTCAGTATTTGGAGGAAAACCACTTCAT | |
| | CCACCGAGACATTGCTGCCAGAAACTGCCTCTTGACCTGTCCAGGCCCTGGAAGAGTG | |
| | GCCAAGATTGGAGACTTCGGGATGGCCCGAGACATCTACAGGGCGAGCTACTATAGAA | |
| | AGGGAGGCTGTGCCATGCTGCCAGTTAAGTGGATGCCCCCAGAGGCCTTCATGGAAGG | |
| | AATATTCACTTCTAAAACAGACACATGGTCCTTTGGAGTGCTGCTATGGGAAATCTTT | |
| | TCTCTTGGATATATGCCATACCCCAGCAAAAGCAACCAGGAAGTTCTGGAGTTTGTCA | |
| | CCAGTGGAGGCCGGATGGACCCACCCAAGAACTGCCCTGGGCCTGTATACCGGATAAT | |
| | GACTCAGTGCTGGCAACATCAGCCTGAAGACAGGCCCAACTTTGCCATCATTTTGGAG | |
| | AGGATTGAATACTGCACCCAGGACCCGGATGTAATCAACACCGCTTTGCCGATAGAAT | |
| | ATGGTCCACTTGTGGAAGAGGAAGAGAAAGTGCCTGTGAGGCCCAAGGACCCTGAGGG | |
| | GGTTCCTCCTCTCCTGGTCTCTCAACAGGCAAAACGGGAGGAGGAGCGCAGCCCAGCT | |
| | GCCCCACCACCTCTGCCTACCACCTCCTCTGGCAAGGCTGCAAAGAAACCCACAGCTG | |
| | CAGAGATCTCTGTTCGAGTCCCTAGAGGGCCGGCCGTGGAAGGGGGACACGTGAATAT | |
| | GGCATTCTCTCAGTCCAACCCTCCTTCGGAGTTGCACAAGGTCCACGGATCCAGAAAC | |
| | AAGCCCACCAGCTTGTGGAACCCAACGTACGGCTCCTGGTTTACAGAGAAACCCACCA | |
| | AAAAGAATAATCCTATAGCAAAGAAGGAGCCACACGACAGGGGTAACCTGGGGCTGGA | |
| | GGGAAGCTGTACTGTCCCACCTAACGTTGCAACTGGGAGACTTCCGGGGGCCTCACTG | |
| | CTCCTAGAGCCCTCTTCGCTGACTGCCAATATGAAGGAGGTACCTCTGTTCAGGCTAC | |
| | GTCACTTCCCTTGTGGGAATGTCAATTACGGCTACCAGCAACAGGGCTTGCCCTTAGA | |
| | AGCCGCTACTGCCCCTGGAGCTGGTCATTACGAGGATACCATTCTGAAAAGCAAGAAT | |
| | AGCATGAACCAGCCTGGGCCCTGA | |
| ALK- | ATGGCTGTGGCTAGCGATTTCTACCTGCGCTACTACGTAGGGCACAAGGGCAAGTTTG | 41 |
| MAGOHB | GGCACGAGTTTCTGGAGTTCGAATTTCGGCCGGACGTGTACCGCCGGAAGCACCAGGA | |
| | GCTGCAAGCCATGCAGATGGAGCTGCAGAGCCCTGAGTACAAGCTGAGCAAGCTCCGC | |
| | ACCTCGACCATCATGACCGACTACAACCCCAACTACTGCTTTGCTGGCAAGACCTCCT | |
| | CCATCAGTGACCTGAAGGAGGTGCCGCGGAAAAACATCACCCTCATTCGGGGTCTGGG | |
| | CCATGGCGCCTTTGGGGAGGTGTATGAAGGCCAGGTGTCCGGAATGCCCAACGACCCA | |
| | AGCCCCCTGCAAGTGGCTGTGAAGACGCTGCCTGAAGTGTGCTCTGAACAGGACGAAC | |
| | TGGATTTCCTCATGGAAGCCCTGATCATCAGCAAATTCAACCACCAGAACATTGTTCG | |
| | CTGCATTGGGGTGAGCCTGCAATCCCTGCCCCGGTTCATCCTGCTGGAGCTCATGGCG | |
| | GGGGGAGACCTCAAGTCCTTCCTCCGAGAGACCCGCCCTCGCCCGAGCCAGCCCTCCT | |
| | CCCTGGCCATGCTGGACCTTCTGCACGTGGCTCGGGACATTGCCTGTGGCTGTCAGTA | |
| | TTTGGAGGAAAACCACTTCATCCACCGAGACATTGCTGCCAGAAACTGCCTCTTGACC | |
| | TGTCCAGGCCCTGGAAGAGTGGCCAAGATTGGAGACTTCGGGATGGCCCGAGACATCT | |
| | ACAGGGCGAGCTACTATAGAAAGGGAGGCTGTGCCATGCTGCCAGTTAAGTGGATGCC | |
| | CCCAGAGGCCTTCATGGAAGGAATATTCACTTCTAAAACAGACACATGGTCCTTTGGA | |
| | GTGCTGCTATGGGAAATCTTTTCTCTTGGATATATGCCATACCCCAGCAAAAGCAACC | |
| | AGGAAGTTCTGGAGTTTGTCACCAGTGGAGGCCGGATGGACCCACCCAAGAACTGCCC | |
| | TGGGCCTGTATACCGGATAATGACTCAGTGCTGGCAACATCAGCCTGAAGACAGGCCC | |
| | AACTTTGCCATCATTTTGGAGAGGATTGAATACTGCACCCAGGACCCGGATGTAATCA | |
| | ACACCGCTTTGCCGATAGAATATGGTCCACTTGTGGAAGAGGAAGAGAAAGTGCCTGT | |
| | GAGGCCCAAGGACCCTGAGGGGGTTCCTCCTCTCCTGGTCTCTCAACAGGCAAAACGG | |
| | GAGGAGGAGCGCAGCCCAGCTGCCCCACCACCTCTGCCTACCACCTCCTCTGGCAAGG | |
| | CTGCAAAGAAACCCACAGCTGCAGAGATCTCTGTTCGAGTCCCTAGAGGGCCGGCCGT | |
| | GGAAGGGGGACACGTGAATATGGCATTCTCTCAGTCCAACCCTCCTTCGGAGTTGCAC | |
| | AAGGTCCACGGATCCAGAAACAAGCCCACCAGCTTGTGGAACCCAACGTACGGCTCCT | |
| | GGTTTACAGAGAAACCCACCAAAAAGAATAATCCTATAGCAAAGAAGGAGCCACACGA | |
| | CAGGGGTAACCTGGGGCTGGAGGGAAGCTGTACTGTCCCACCTAACGTTGCAACTGGG | |
| | AGACTTCCGGGGGCCTCACTGCTCCTAGAGCCCTCTTCGCTGACTGCCAATATGAAGG | |
| | AGGTACCTCTGTTCAGGCTACGTCACTTCCCTTGTGGGAATGTCAATTACGGCTACCA | |
| | GCAACAGGGCTTGCCCTTAGAAGCCGCTACTGCCCCTGGAGCTGGTCATTACGAGGAT | |
| | ACCATTCTGAAAAGCAAGAATAGCATGAACCAGCCTGGGCCCTGA | |
| ALK- | ATGATGAGCTTTGTGCAAAAGGGGAGCTGGCTACTTCTCGCTCTGCTTCATCCCACTA | 43 |
| COL3A1 | TTATTTTGGCACAACAGGAAGCTGTTGAAGGAGGATGTTCCCATCTTGGTCAGTCCTA | |
| | TGCGGATAGAGATGTCTGGAAGCCAGAACCATGCCAAATATGTGTCTGTGACTCAGGA | |
| | TCCGTTCTCTGCGATGACATAATATGTGACGATCAAGAATTAGACTGCCCCAACCCAG | |
| | AAATTCCATTTGGAGAATGTTGTGCAGTTTGCCCACAGCCTCCAACTGCTCCTACTCG | |
| | CCCTCCTAATGGTCAAGGACCTCAAGGCCCCAAGGGAGATCCAGGCCCTCCTGGTATT | |
| | CCTGGGAGAAATGGTGACCCTGGTATTCCAGGACAACCAGGGTCCCCTGGTTCTCCTG | |
| | GCCCCCCTGGAATCTGTGAATCATGCCCTACTGGTCCTCAGAACTATTCTCCCCAGTA | |
| | TGATTCATATGATGTCAAGTCTGGAGTAGCAGTAGGAGGACTCGCAGGCTATCCTGGA | |
| | CCAGCTGGCCCCCCAGGCCCTCCCGGTCCCCCTGGTACATCTGGTCATCCTGGTTCCC | |
| | CTGGATCTCCAGGATACCAAGGACCCCCTGGTGAACCTGGGCAAGCTGGTCCTTCAGG | |
| | CCCTCCAGGACCTCCTGGTGCTATAGGTCCATCTGGTCCTGCTGGAAAAGATGGAGAA | |
| | TCAGGTAGACCCGGACGACCTGGAGAGCGAGGATTGCCTGGACCTCCAGGTATCAAAG | |
| | GTCCAGCTGGGATACCTGGATTCCCTGGTATGAAAGGACACAGAGGCTTCGATGGACG | |
| | AAATGGAGAAAAGGGTGAAACAGGTGCTCCTGGATTAAAGGGTGAAAATGGTCTTCCA | |
| | GGCGAAAATGGAGCTCCTGGACCCATGGGTCCAAGAGGGGCTCCTGGTGAGCGAGGAC | |
| | GGCCAGGACTTCCTGGGGCTGCAGGTGCTCGGGGTAATGACGGTGCTCGAGGCAGTGA | |
| | TGGTCAACCAGGCCCTCCTGGTCCTCCTGGAACTGCCGGATTCCCTGGATCCCCTGGT | |
| | GCTAAGGGTGAAGTTGGACCTGCAGGGTCTCCTGGTTCAAATGGTGCCCCTGGACAAA | |
| | GAGGAGAACCTGGACCTCAGGGACACGCTGGTGCTCAAGGTCCTCCTGGCCCTCCTGG | |
| | GATTAATGGTAGTCCTGGTGGTAAAGGCGAAATGGGTCCCGCTGGCATTCCTGGAGCT | |
| | CCTGGACTGATGGGAGCCCGGGGTCCTCCAGGACCAGCCGGTGCTAATGGTGCTCCTG | |
| | GACTGCGAGGTGGTGCAGGTGAGCCTGGTAAGAATGGTGCCAAAGGAGAGCCCGGACC | |
| | ACGTGGTGAACGCGGTGAGGCTGGTATTCCAGGTGTTCCAGGAGCTAAAGGCGAAGAT | |
| | GGCAAGGATGGATCACCTGGAGAACCTGGTGCAAATGGGCTTCCAGGAGCTGCAGGAG | |
| | AAAGGGGTGCCCCTGGGTTCCGAGGACCTGCTGGACCAAATGGCATCCCAGGAGAAAA | |
| | GGGTCCTGCTGGAGAGCGTGGTGCTCCAGGCCCTGCAGGGCCCAGAGGAGCTGCTGGA | |
| | GAACCTGGCAGAGATGGCGTCCCTGGAGGTCCAGGAATGAGGGGCATGCCCGGAAGTC | |
| | CAGGAGGACCAGGAAGTGATGGGAAACCAGGGCCTCCCGGAAGTCAAGGAGAAAGTGG | |
| | TCGACCAGGTCCTCCTGGGCCATCTGGTCCCCGAGGTCAGCCTGGTGTCATGGGCTTC | |
| | CCCGGTCCTAAAGGAAATGATGGTGCTCCTGGTAAGAATGGAGAACGAGGTGGCCCTG | |
| | GAGGACCTGGCCCTCAGGGTCCTCCTGGAAAGAATGGTGAAACTGGACCTCAGGGACC | |
| | CCCAGGGCCTACTGGGCCTGGTGGTGACAAAGGAGACACAGGACCCCCTGGTCCACAA | |
| | GGATTACAAGGCTTGCCTGGTACAGGTGGTCCTCCAGGAGAAAATGGAAAACCTGGGG | |
| | AACCAGGTCCAAAGGGTGATGCCGGTGCACCTGGAGCTCCAGGAGGCAAGGGTGATGC | |
| | TGGTGCCCCTGGTGAACGTGGACCTCCTGGATTGGCAGGGGCCCCAGGACTTAGAGGT | |
| | GGAGCTGGTCCCCCTGGTCCCGAAGGAGGAAAGGGTGCTGCTGGTCCTCCTGGGCCAC | |
| | CTGGTGCTGCTGGTACTCCTGGTCTGCAAGGAATGCCTGGAGAAAGAGGAGGTCTTGG | |
| | AAGTCCTGGTCCAAAGGGTGACAAGGGTGAACCAGGCGGTCCAGGTGCTGATGGTGTC | |
| | CCAGGGAAAGATGGCCCAAGGGGTCCTACTGGTCCTATTGGTCCTCCTGGCCCAGCTG | |
| | GCCAGCCTGGAGATAAGGGTGAAGGTGGTGCCCCCGGACTTCCAGGTATAGCTGGACC | |
| | TCGTGGTAGCCCTGGTGAGAGAGGTGAAACTGGCCCTCCAGGACCTGCTGGTTTCCCT | |
| | GGTGCTCCTGGACAGAATGGTGAACCTGGTGGTAAAGGAGAAAGAGGGGCTCCGGGTG | |
| | AGAAAGGTGAAGGAGGCCCTCCTGGAGTTGCAGGACCCCCTGGAGGTTCTGGACCTGC | |
| | TGGTCCTCCTGGTCCCCAAGGTGTCAAAGGTGAACGTGGCAGTCCTGGTGGACCTGGT | |
| | GCTGCTGGCTTCCCTGGTGCTCGTGGTCTTCCTGGTCCTCCTGGTAGTAATGGTAACC | |
| | CAGGACCCCCAGGTCCCAGCGGTTCTCCAGGCAAGGATGGGCCCCCAGGTCCTGCGGG | |
| | TAACACTGGTGCTCCTGGCAGCCCTGGAGTGTCTGGACCAAAAGGTGATGCTGGCCAA | |
| | CCAGGAGAGAAGGGATCGCCTGGTGCCCAGGGCCCACCAGGAGCTCCAGGCCCACTTG | |
| | GGATTGCTGGGATCACTGGAGCACGGGGTCTTGCAGGACCACCAGGCATGCCAGGTCC | |
| | TAGGGGAAGCCCTGGCCCTCAGGGTGTCAAGGGTGAAAGTGGGAAACCAGGAGCTAAC | |
| | GGTCTCAGTGGAGAACGTGGTCCCCCTGGACCCCAGGGTCTTCCTGGTCTGGCTGGTA | |
| | CAGCTGGTGAACCTGGAAGAGATGGAAACCCTGGATCAGATGGTCTTCCAGGCCGAGA | |
| | TGGATCTCCTGGTGGCAAGGGTGATCGTGGTGAAAATGGCTCTCCTGGTGCCCCTGGC | |
| | GCTCCTGGTCATCCAGGCCCACCTGGTCCTGTCGGTCCAGCTGGAAAGAGTGGTGACA | |
| | GAGGAGAAAGTGGCCCTGCTGGCCCTGCTGGTGCTCCCGGTCCTGCTGGTTCCCGAGG | |
| | TGCTCCTGGTCCTCAAGGCCCACGTGGTGACAAAGGTGAAACAGGTGAACGTGGAGCT | |
| | GCTGGCATCAAAGGACATCGAGGATTCCCTGGTAATCCAGGTGCCCCAGGTTCTCCAG | |
| | GCCCTGCTGGTCAGCAGGGTGCAATCGGCAGTCCAGGACCTGCAGGCCCCAGAGGACC | |
| | TGTTGGACCCAGTGGACCTCCTGGCAAAGATGGAACCAGTGGACATCCAGGTCCCATT | |
| | GGACCACCAGGGCCTCGAGGTAACAGAGGTGAAAGAGGATCTGAGGGCTCCCCAGGCC | |
| | ACCCAGGGCAACCAGGCCCTCCTGGACCTCCTGGTGCCCCTGGTCCTTGCTGTGGTGG | |
| | TGTTGGAGCCGCTGCCATTGCTGGGATTGGAGGTGAAAAAGCTGGCGGTTTTGCCCCG | |
| | TATTATGGAGATGAACCAATGGATTTCAAAATCAACACCGATGAGATTATGACTTCAC | |
| | TCAAGTCTGTTAATGGACAAATAGAAAGCCTCATTAGTCCTGATGGTTCTCGTAAAAA | |
| | CCCCGCTAGAAACTGCAGAGACCTGAAATTCTGCCATCCTGAACTCAAGAGTGTGTCA | |
| | CCCACCCCGGAGCCACACCTGCCACTCTCGCTGATCCTCTCTGTGGTGACCTCTGCCC | |
| | TCGTGGCCGCCCTGGTCCTGGCTTTCTCCGGCATCATGATTGTGTACCGCCGGAAGCA | |
| | CCAGGAGCTGCAAGCCATGCAGATGGAGCTGCAGAGCCCTGAGTACAAGCTGAGCAAG | |
| | CTCCGCACCTCGACCATCATGACCGACTACAACCCCAACTACTGCTTTGCTGGCAAGA | |
| | CCTCCTCCATCAGTGACCTGAAGGAGGTGCCGCGGAAAAACATCACCCTCATTCGGGG | |
| | TCTGGGCCATGGCGCCTTTGGGGAGGTGTATGAAGGCCAGGTGTCCGGAATGCCCAAC | |
| | GACCCAAGCCCCCTGCAAGTGGCTGTGAAGACGCTGCCTGAAGTGTGCTCTGAACAGG | |
| | ACGAACTGGATTTCCTCATGGAAGCCCTGATCATCAGCAAATTCAACCACCAGAACAT | |
| | TGTTCGCTGCATTGGGGTGAGCCTGCAATCCCTGCCCCGGTTCATCCTGCTGGAGCTC | |
| | ATGGCGGGGGGAGACCTCAAGTCCTTCCTCCGAGAGACCCGCCCTCGCCCGAGCCAGC | |
| | CCTCCTCCCTGGCCATGCTGGACCTTCTGCACGTGGCTCGGGACATTGCCTGTGGCTG | |
| | TCAGTATTTGGAGGAAAACCACTTCATCCACCGAGACATTGCTGCCAGAAACTGCCTC | |
| | TTGACCTGTCCAGGCCCTGGAAGAGTGGCCAAGATTGGAGACTTCGGGATGGCCCGAG | |
| | ACATCTACAGGGCGAGCTACTATAGAAAGGGAGGCTGTGCCATGCTGCCAGTTAAGTG | |
| | GATGCCCCCAGAGGCCTTCATGGAAGGAATATTCACTTCTAAAACAGACACATGGTCC | |
| | TTTGGAGTGCTGCTATGGGAAATCTTTTCTCTTGGATATATGCCATACCCCAGCAAAA | |
| | GCAACCAGGAAGTTCTGGAGTTTGTCACCAGTGGAGGCCGGATGGACCCACCCAAGAA | |
| | CTGCCCTGGGCCTGTATACCGGATAATGACTCAGTGCTGGCAACATCAGCCTGAAGAC | |
| | AGGCCCAACTTTGCCATCATTTTGGAGAGGATTGAATACTGCACCCAGGACCCGGATG | |
| | TAATCAACACCGCTTTGCCGATAGAATATGGTCCACTTGTGGAAGAGGAAGAGAAAGT | |
| | GCCTGTGAGGCCCAAGGACCCTGAGGGGGTTCCTCCTCTCCTGGTCTCTCAACAGGCA | |
| | AAACGGGAGGAGGAGCGCAGCCCAGCTGCCCCACCACCTCTGCCTACCACCTCCTCTG | |
| | GCAAGGCTGCAAAGAAACCCACAGCTGCAGAGATCTCTGTTCGAGTCCCTAGAGGGCC | |
| | GGCCGTGGAAGGGGGACACGTGAATATGGCATTCTCTCAGTCCAACCCTCCTTCGGAG | |
| | TTGCACAAGGTCCACGGATCCAGAAACAAGCCCACCAGCTTGTGGAACCCAACGTACG | |
| | GCTCCTGGTTTACAGAGAAACCCACCAAAAAGAATAATCCTATAGCAAAGAAGGAGCC | |
| | ACACGACAGGGGTAACCTGGGGCTGGAGGGAAGCTGTACTGTCCCACCTAACGTTGCA | |
| | ACTGGGAGACTTCCGGGGGCCTCACTGCTCCTAGAGCCCTCTTCGCTGACTGCCAATA | |
| | TGAAGGAGGTACCTCTGTTCAGGCTACGTCACTTCCCTTGTGGGAATGTCAATTACGG | |
| | CTACCAGCAACAGGGCTTGCCCTTAGAAGCCGCTACTGCCCCTGGAGCTGGTCATTAC | |
| | GAGGATACCATTCTGAAAAGCAAGAATAGCATGAACCAGCCTGGGCCCTGA | |

In some embodiments, the ALK-COL3A1 fusion nucleic acid molecule of the disclosure comprises the nucleotide sequence of SEQ ID NO: 1, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-CDC42BPA fusion nucleic acid molecule of the disclosure comprises the nucleotide sequence of SEQ ID NO: 2, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-EPHA2 fusion nucleic acid molecule of the disclosure comprises the nucleotide sequence of SEQ ID NO: 3, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MYO5C fusion nucleic acid molecule of the disclosure comprises the nucleotide sequence of SEQ ID NO: 4, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TRIM24 fusion nucleic acid molecule of the disclosure comprises the nucleotide sequence of SEQ ID NO: 5, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-SKAP1 fusion nucleic acid molecule of the disclosure comprises the nucleotide sequence of SEQ ID NO: 6, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-UBE3B fusion nucleic acid molecule of the disclosure comprises the nucleotide sequence of SEQ ID NO: 7, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TNS3 fusion nucleic acid molecule of the disclosure comprises the nucleotide sequence of SEQ ID NO: 8, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-C2orf73 fusion nucleic acid molecule of the disclosure comprises the nucleotide sequence of SEQ ID NO: 9, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-AZI2 fusion nucleic acid molecule of the disclosure comprises the nucleotide sequence of SEQ ID NO: 10, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MANBA fusion nucleic acid molecule of the disclosure comprises the nucleotide sequence of SEQ ID NO: 11, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-CNTNAP5 fusion nucleic acid molecule of the disclosure comprises the nucleotide sequence of SEQ ID NO: 12, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TANGO6 fusion nucleic acid molecule of the disclosure comprises the nucleotide sequence of SEQ ID NO: 14, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-NFIA fusion nucleic acid molecule of the disclosure comprises the nucleotide sequence of SEQ ID NO: 15, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-RPS6KA5 fusion nucleic acid molecule of the disclosure comprises the nucleotide sequence of SEQ ID NO: 16, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TG fusion nucleic acid molecule of the disclosure comprises the nucleotide sequence of SEQ ID NO: 17, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-LRRFIP2 fusion nucleic acid molecule of the disclosure comprises the nucleotide sequence of SEQ ID NO: 18, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MYO5C fusion nucleic acid molecule of the disclosure comprises the nucleotide sequence of SEQ ID NO: 19, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-AGAP1 fusion nucleic acid molecule of the disclosure comprises the nucleotide sequence of SEQ ID NO: 20, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MED13L fusion nucleic acid molecule of the disclosure comprises the nucleotide sequence of SEQ ID NO: 21, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MTBP fusion nucleic acid molecule of the disclosure comprises the nucleotide sequence of SEQ ID NO: 22, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-SLC30A6 fusion nucleic acid molecule of the disclosure comprises the nucleotide sequence of SEQ ID NO: 24, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-GMCL1 fusion nucleic acid molecule of the disclosure comprises the nucleotide sequence of SEQ ID NO: 25, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-AGAP1 fusion nucleic acid molecule of the disclosure comprises the nucleotide sequence of SEQ ID NO: 26, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-ZNF454 fusion nucleic acid molecule of the disclosure comprises the nucleotide sequence of SEQ ID NO: 27, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TTC28 fusion nucleic acid molecule of the disclosure comprises the nucleotide sequence of SEQ ID NO: 28, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-NINJ2 fusion nucleic acid molecule of the disclosure comprises the nucleotide sequence of SEQ ID NO: 29, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-UTRN fusion nucleic acid molecule of the disclosure comprises the nucleotide sequence of SEQ ID NO: 30, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-ACTN4 fusion nucleic acid molecule of the disclosure comprises the nucleotide sequence of SEQ ID NO: 31, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-CPSF7 fusion nucleic acid molecule of the disclosure comprises the nucleotide sequence of SEQ ID NO: 32, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-SLC25A13 fusion nucleic acid molecule of the disclosure comprises the nucleotide sequence of SEQ ID NO: 33, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-CTNND1 fusion nucleic acid molecule of the disclosure comprises the nucleotide sequence of SEQ ID NO: 34, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-KLC4 fusion nucleic acid molecule of the disclosure comprises the nucleotide sequence of SEQ ID NO: 35, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-BTBD9 fusion nucleic acid molecule of the disclosure comprises the nucleotide sequence of SEQ ID NO: 36, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

In some embodiments, the ALK-CPQ fusion nucleic acid molecule of the disclosure comprises the nucleotide sequence of SEQ ID NO: 38, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-KIFSC fusion nucleic acid molecule of the disclosure comprises the nucleotide sequence of SEQ ID NO: 40, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MAGOHB fusion nucleic acid molecule of the disclosure comprises the nucleotide sequence of SEQ ID NO: 41, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-COL3A1 fusion nucleic acid molecule of the disclosure comprises the nucleotide sequence of SEQ ID NO: 43, or a nucleotide sequence with at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

In some embodiments of any of the ALK fusion nucleic acid molecules provided herein, the fusion nucleic acid molecule is a genomic nucleic acid molecule (i.e., genomic DNA or fragments thereof), or a transcribed nucleic acid molecule, e.g., an RNA such as mRNA, or a cDNA, or fragments thereof.

### (ii) ALK Fusion Polypeptides

In certain aspects, provided herein are ALK fusion polypeptides which comprise at least a portion of an ALK polypeptide and at least a portion of a polypeptide encoded by another gene. In some embodiments, an ALK fusion polypeptide of the disclosure is a fusion polypeptide encoded by any of the ALK fusion nucleic acid molecules provided herein, or a portion thereof.

In some aspects, provided herein are ALK fusion polypeptides that comprise at least a portion of an ALK polypeptide and at least a portion of a polypeptide encoded by another gene, e.g., an ABCB11, ACTN4, AGAP1, APH1A, AZI2, BTBD9, C2orf73, CAPN14, CARMIL1, CASP8, CDC42BPA, CIB4, CNTNAP5, COL3A1, CPQ, CPSF7, CREBBP, CTBP1, CTNND1, CYP51A1, CYS1, EPHA2, FHOD3, FILIP1L, GMCL1, GPN1, GPR113, HADHA, HS1BP3, INTS9, ITGA6, KCTD18, KIF5C, KLC4, LINC00535, LRRFIP2, MAGOHB, MAMDC4, MANBA, MAP3K9, MED13L, METTL25, MTBP, MYH10, MYO5C, NFIA, NINJ2, OPRM1, OTX1, PAQR4, PDCD10, PDE3A, PELI1, PLEC, PTGER4, PTPRJ, QKI, RPS6KA5, SASH1, SEC16B, SKAP1, SLC25A13, SLC30A6, SNX17, SOX13, SRSF7, TANGO6, TG, TMCO3, TNS3, TRIM24, TTC28, UBE2L3, UBE3B, UTRN, VASP, WDR92, YPEL5, ZNF446, ZNF454, ZNF513, or ZSWIM2 gene, or a gene listed in Table 1. For example, in some embodiments, provided herein are fusion polypeptides encoded by an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule of the disclosure. In some embodiments, provided herein are ALK fusion polypeptides encoded by any of the ALK fusion nucleic acid molecules as described herein and/or in any of Tables 1-7, and/or in the Examples herein. In some embodiments, the ALK fusion polypeptide comprises an ALK kinase domain, or a fragment of an ALK kinase domain having ALK kinase activity. In some embodiments, the ALK fusion polypeptide has ALK kinase activity. In some embodiments, the kinase activity is constitutive. In some embodiments, the ALK fusion polypeptide is oncogenic. In some embodiments, the ALK fusion polypeptide promotes cancer cell survival, angiogenesis, cancer cell proliferation, and any combination thereof.

In some embodiments, the ALK-ABCB11 fusion polypeptide of the disclosure comprises, in the amino-terminus to carboxyl-terminus direction, an ABCB11 polypeptide or a portion thereof fused to an ALK polypeptide or a portion thereof. In some embodiments, the ALK-ACTN4 fusion polypeptide of the disclosure comprises, in the amino-terminus to carboxyl-terminus direction, an ACTN4 polypeptide or a portion thereof fused to an ALK polypeptide or a portion thereof. In some embodiments, the ALK-AGAP1 fusion polypeptide of the disclosure comprises, in the amino-terminus to carboxyl-terminus direction, an AGAP1 polypeptide or a portion thereof fused to an ALK polypeptide or a portion thereof. In some embodiments, the ALK-AGAP1 fusion polypeptide of the disclosure comprises, in the amino-terminus to carboxyl-terminus direction, an ALK polypeptide or a portion thereof fused to an AGAP1 polypeptide or a portion thereof. In some embodiments, the ALK-APH1A fusion polypeptide of the disclosure comprises, in the amino-terminus to carboxyl-terminus direction, an ALK polypeptide or a portion thereof fused to an APH1A polypeptide or a portion thereof. In some embodiments, the ALK-AZI2 fusion polypeptide of the disclosure comprises, in the amino-terminus to carboxyl-terminus direction, an AZI2 polypeptide or a portion thereof fused to an ALK polypeptide or a portion thereof. In some embodiments, the ALK-BTBD9 fusion polypeptide of the disclosure comprises, in the amino-terminus to carboxyl-terminus direction, a BTBD9 polypeptide or a portion thereof fused to an ALK polypeptide or a portion thereof. In some embodiments, the ALK-C2orf73 fusion polypeptide of the disclosure comprises, in the amino-terminus to carboxyl-terminus direction, a C2orf73 polypeptide or a portion thereof fused to an ALK polypeptide or a portion thereof. In some embodiments, the ALK-CAPN14 fusion polypeptide of the disclosure comprises, in the amino-terminus to carboxyl-terminus direction, an ALK polypeptide or a portion thereof fused to a CAPN14 polypeptide or a portion thereof. In some embodiments, the ALK-CARMIL1 fusion polypeptide of the disclosure comprises, in the amino-terminus to carboxyl-terminus direction, a CARMIL1 polypeptide or a portion thereof fused to an ALK polypeptide or a portion thereof. In some embodiments, the ALK-CASP8 fusion polypeptide of the disclosure comprises, in the amino-terminus to carboxyl-terminus direction, a CASP8 polypeptide or a portion thereof fused to an ALK polypeptide or a portion thereof. In some embodiments, the ALK-CDC42BPA fusion polypeptide of the disclosure comprises, in the amino-terminus to carboxyl-terminus direction, a CDC42BPA polypeptide or a portion thereof fused to an ALK polypeptide or a portion thereof. In some embodiments, the ALK-CIB4 fusion polypeptide of the disclosure comprises, in the amino-terminus to carboxyl-terminus direction, a CIB4 polypeptide or a portion thereof fused to an ALK polypeptide or a portion thereof. In some embodiments, the ALK-CNTNAP5 fusion polypeptide of the disclosure comprises, in the amino-terminus to carboxyl-terminus direction, a CNTNAP5 polypeptide or a portion thereof fused to an ALK polypeptide or a portion thereof. In some embodiments, the ALK-COL3A1 fusion polypeptide of the disclosure comprises, in the amino-terminus to carboxyl-terminus direction, a COL3A1 polypeptide or a portion thereof fused to an ALK polypeptide or a portion thereof. In some embodiments, the ALK-CPQ fusion polypeptide of the disclosure comprises, in the amino-terminus to carboxyl-terminus direction, a CPQ polypeptide or a portion thereof fused to an ALK polypeptide or a portion thereof. In some embodiments, the ALK-CPSF7 fusion polypeptide of the disclosure comprises, in the amino-terminus to carboxyl-terminus direction, a CPSF7 polypeptide or a portion thereof fused to an ALK polypeptide or a portion thereof. In some embodiments, the ALK-CREBBP fusion polypeptide of the disclosure comprises, in the amino-terminus to carboxyl-terminus direction, an ALK polypeptide or a portion thereof fused to a CREBBP polypeptide or a portion thereof. In some embodiments, the ALK-CTBP1 fusion polypeptide of the disclosure comprises, in the amino-terminus to carboxyl-terminus direction, a CTBP1 polypeptide or a portion thereof fused to an ALK polypeptide or a portion thereof. In some embodiments, the ALK-CTNND1 fusion polypeptide of the disclosure comprises, in the amino-terminus to carboxyl-terminus direction, a CTNND1 polypeptide or a portion thereof fused to an ALK polypeptide or a portion thereof. In some embodiments, the ALK-CYP51A1 fusion polypeptide of the disclosure comprises, in the amino-terminus to carboxyl-terminus direction, a CYP51A1 polypeptide or a portion thereof fused to an ALK polypeptide or a portion thereof. In some embodiments, the ALK-CYS1 fusion polypeptide of the disclosure comprises, in the amino-terminus to carboxyl-terminus direction, a CYS1 polypeptide or a portion thereof fused to an ALK polypeptide or a portion thereof. In some embodiments, the ALK-EPHA2 fusion polypeptide of the disclosure comprises, in the amino-terminus to carboxyl-terminus direction, an EPHA2 polypeptide or a portion thereof fused to an ALK polypeptide or a portion thereof. In some embodiments, the ALK-FHOD3 fusion polypeptide of the disclosure comprises, in the amino-terminus to carboxyl-terminus direction, an ALK polypeptide or a portion thereof fused to a FHOD3 polypeptide or a portion thereof. In some embodiments, the ALK-GMCL1 fusion polypeptide of the disclosure comprises, in the amino-terminus to carboxyl-terminus direction, an ALK polypeptide or a portion thereof fused to a GMCL1 polypeptide or a portion thereof. In some embodiments, the ALK-GPN1 fusion polypeptide of the disclosure comprises, in the amino-terminus to carboxyl-terminus direction, a GPN1 polypeptide or a portion thereof fused to an ALK polypeptide or a portion thereof. In some embodiments, the ALK-GPR113 fusion polypeptide of the disclosure comprises, in the amino-terminus to carboxyl-terminus direction, a GPR113 polypeptide or a portion thereof fused to an ALK polypeptide or a portion thereof. In some embodiments, the ALK-HADHA fusion polypeptide of the disclosure comprises, in the amino-terminus to carboxyl-terminus direction, a HADHA polypeptide or a portion thereof fused to an ALK polypeptide or a portion thereof. In some embodiments, the ALK-HS1BP3 fusion polypeptide of the disclosure comprises, in the amino-terminus to carboxyl-terminus direction, an ALK polypeptide or a portion thereof fused to an HS1BP3 polypeptide or a portion thereof. In some embodiments, the ALK-INTS9 fusion polypeptide of the disclosure comprises, in the amino-terminus to carboxyl-terminus direction, an INTS9 polypeptide or a portion thereof fused to an ALK polypeptide or a portion thereof. In some embodiments, the ALK-ITGA6 fusion polypeptide of the disclosure comprises, in the amino-terminus to carboxyl-terminus direction, an ALK polypeptide or a portion thereof fused to an ITGA6 polypeptide or a portion thereof. In some embodiments, the ALK-KCTD18 fusion polypeptide of the disclosure comprises, in the amino-terminus to carboxyl-terminus direction, an ALK polypeptide or a portion thereof fused to a KCTD18 polypeptide or a portion thereof. In some embodiments, the ALK-KIF5C fusion polypeptide of the disclosure comprises, in the amino-terminus to carboxyl-terminus direction, a KIF5C polypeptide or a portion thereof fused to an ALK polypeptide or a portion thereof. In some embodiments, the ALK-KLC4 fusion polypeptide of the disclosure comprises, in the amino-terminus to carboxyl-terminus direction, a KLC4 polypeptide or a portion thereof fused to an ALK polypeptide or a portion thereof. In some embodiments, the ALK-LINC00535 fusion polypeptide of the disclosure comprises an ALK polypeptide or a portion thereof. In some embodiments, the ALK-LRRFIP2 fusion polypeptide of the disclosure comprises, in the amino-terminus to carboxyl-terminus direction, an LRRFIP2 polypeptide or a portion thereof fused to an ALK polypeptide or a portion thereof. In some embodiments, the ALK-MAGOHB fusion polypeptide of the disclosure comprises, in the amino-terminus to carboxyl-terminus direction, a MAGOHB polypeptide or a portion thereof fused to an ALK polypeptide or a portion thereof. In some embodiments, the ALK-MAMDC4 fusion polypeptide of the disclosure comprises, in the amino-terminus to carboxyl-terminus direction, an ALK polypeptide or a portion thereof fused to a MAMDC4 polypeptide or a portion thereof. In some embodiments, the ALK-MANBA fusion polypeptide of the disclosure comprises, in the amino-terminus to carboxyl-terminus direction, a MANBA polypeptide or a portion thereof fused to an ALK polypeptide or a portion thereof. In some embodiments, the ALK-MAP3K9 fusion polypeptide of the disclosure comprises, in the amino-terminus to carboxyl-terminus direction, an ALK polypeptide or a portion thereof fused to a MAP3K9 polypeptide or a portion thereof. In some embodiments, the ALK-MED13L fusion polypeptide of the disclosure comprises, in the amino-terminus to carboxyl-terminus direction, an ALK polypeptide or a portion thereof fused to an MED13L polypeptide or a portion thereof. In some embodiments, the ALK-METTL25 fusion polypeptide of the disclosure comprises, in the amino-terminus to carboxyl-terminus direction, a METTL25 polypeptide or a portion thereof fused to an ALK polypeptide or a portion thereof. In some embodiments, the ALK-MTBP fusion polypeptide of the disclosure comprises, in the amino-terminus to carboxyl-terminus direction, an ALK polypeptide or a portion thereof fused to an MTBP polypeptide or a portion thereof. In some embodiments, the ALK-MYH10 fusion polypeptide of the disclosure comprises, in the amino-terminus to carboxyl-terminus direction, an MYH10 polypeptide or a portion thereof fused to an ALK polypeptide or a portion thereof. In some embodiments, the ALK-MYO5C fusion polypeptide of the disclosure comprises, in the amino-terminus to carboxyl-terminus direction, a MYO5C polypeptide or a portion thereof fused to an ALK polypeptide or a portion thereof. In some embodiments, the ALK-NFIA fusion polypeptide of the disclosure comprises, in the amino-terminus to carboxyl-terminus direction, an NFIA polypeptide or a portion thereof fused to an ALK polypeptide or a portion thereof. In some embodiments, the ALK-NINJ2 fusion polypeptide of the disclosure comprises, in the amino-terminus to carboxyl-terminus direction, a NINJ2 polypeptide or a portion thereof fused to an ALK polypeptide or a portion thereof. In some embodiments, the ALK-OPRM1 fusion polypeptide of the disclosure comprises, in the amino-terminus to carboxyl-terminus direction, an OPRM1 polypeptide or a portion thereof fused to an ALK polypeptide or a portion thereof. In some embodiments, the ALK-OTX1 fusion polypeptide of the disclosure comprises, in the amino-terminus to carboxyl-terminus direction, an ALK polypeptide or a portion thereof fused to an OTX1 polypeptide or a portion thereof. In some embodiments, the ALK-PAQR4 fusion polypeptide of the disclosure comprises, in the amino-terminus to carboxyl-terminus direction, an ALK polypeptide or a portion thereof fused to a PAQR4 polypeptide or a portion thereof. In some embodiments, the ALK-PDCD10 fusion polypeptide of the disclosure comprises, in the amino-terminus to carboxyl-terminus direction, an ALK polypeptide or a portion thereof fused to a PDCD10 polypeptide or a portion thereof. In some embodiments, the ALK-PDE3A fusion polypeptide of the disclosure comprises, in the amino-terminus to carboxyl-terminus direction, a PDE3A polypeptide or a portion thereof fused to an ALK polypeptide or a portion thereof. In some embodiments, the ALK-PELI1 fusion polypeptide of the disclosure comprises, in the amino-terminus to carboxyl-terminus direction, a PELI1 polypeptide or a portion thereof fused to an ALK polypeptide or a portion thereof. In some embodiments, the ALK-PELI1 fusion polypeptide of the disclosure comprises, in the amino-terminus to carboxyl-terminus direction, an ALK polypeptide or a portion thereof. In some embodiments, the ALK-PLEC fusion polypeptide of the disclosure comprises, in the amino-terminus to carboxyl-terminus direction, a PLEC polypeptide or a portion thereof fused to an ALK polypeptide or a portion thereof. In some embodiments, the ALK-PTGER4 fusion polypeptide of the disclosure comprises, in the amino-terminus to carboxyl-terminus direction, an ALK polypeptide or a portion thereof fused to a PTGER4 polypeptide or a portion thereof. In some embodiments, the ALK-PTPRJ fusion polypeptide of the disclosure comprises, in the amino-terminus to carboxyl-terminus direction, an ALK polypeptide or a portion thereof fused to a PTPRJ polypeptide or a portion thereof. In some embodiments, the ALK-QKI fusion polypeptide of the disclosure comprises, in the amino-terminus to carboxyl-terminus direction, a QKI polypeptide or a portion thereof fused to an ALK polypeptide or a portion thereof. In some embodiments, the ALK-RPS6KA5 fusion polypeptide of the disclosure comprises, in the amino-terminus to carboxyl-terminus direction, an RPS6KA5 polypeptide or a portion thereof fused to an ALK polypeptide or a portion thereof. In some embodiments, the ALK-SASH1 fusion polypeptide of the disclosure comprises, in the amino-terminus to carboxyl-terminus direction, a SASH1 polypeptide or a portion thereof fused to an ALK polypeptide or a portion thereof. In some embodiments, the ALK-SEC16B fusion polypeptide of the disclosure comprises, in the amino-terminus to carboxyl-terminus direction, a SEC16B polypeptide or a portion thereof fused to an ALK polypeptide or a portion thereof. In some embodiments, the ALK-SKAP1 fusion polypeptide of the disclosure comprises, in the amino-terminus to carboxyl-terminus direction, a SKAP1 polypeptide or a portion thereof fused to an ALK polypeptide or a portion thereof. In some embodiments, the ALK-SLC25A13 fusion polypeptide of the disclosure comprises, in the amino-terminus to carboxyl-terminus direction, an SLC25A13 polypeptide or a portion thereof fused to an ALK polypeptide or a portion thereof. In some embodiments, the ALK-SLC30A6 fusion polypeptide of the disclosure comprises, in the amino-terminus to carboxyl-terminus direction, an ALK polypeptide or a portion thereof fused to an SLC30A6 polypeptide or a portion thereof. In some embodiments, the ALK-SNX17 fusion polypeptide of the disclosure comprises, in the amino-terminus to carboxyl-terminus direction, an ALK polypeptide or a portion thereof fused to an SNX17 polypeptide or a portion thereof. In some embodiments, the ALK-SOX13 fusion polypeptide of the disclosure comprises, in the amino-terminus to carboxyl-terminus direction, a SOX13 polypeptide or a portion thereof fused to an ALK polypeptide or a portion thereof. In some embodiments, the ALK-SRSF7 fusion polypeptide of the disclosure comprises, in the amino-terminus to carboxyl-terminus direction, an SRSF7 polypeptide or a portion thereof fused to an ALK polypeptide or a portion thereof. In some embodiments, the ALK-TANGO6 fusion polypeptide of the disclosure comprises, in the amino-terminus to carboxyl-terminus direction, a TANGO6 polypeptide or a portion thereof fused to an ALK polypeptide or a portion thereof. In some embodiments, the ALK-TG fusion polypeptide of the disclosure comprises, in the amino-terminus to carboxyl-terminus direction, a TG polypeptide or a portion thereof fused to an ALK polypeptide or a portion thereof. In some embodiments, the ALK-TMCO3 fusion polypeptide of the disclosure comprises, in the amino-terminus to carboxyl-terminus direction, a TMCO3 polypeptide or a portion thereof fused to an ALK polypeptide or a portion thereof. In some embodiments, the ALK-TNS3 fusion polypeptide of the disclosure comprises, in the amino-terminus to carboxyl-terminus direction, a TNS3 polypeptide or a portion thereof fused to an ALK polypeptide or a portion thereof. In some embodiments, the ALK-TRIM24 fusion polypeptide of the disclosure comprises, in the amino-terminus to carboxyl-terminus direction, a TRIM24 polypeptide or a portion thereof fused to an ALK polypeptide or a portion thereof. In some embodiments, the ALK-TTC28 fusion polypeptide of the disclosure comprises, in the amino-terminus to carboxyl-terminus direction, a TTC28 polypeptide or a portion thereof fused to an ALK polypeptide or a portion thereof. In some embodiments, the ALK-UBE2L3 fusion polypeptide of the disclosure comprises, in the amino-terminus to carboxyl-terminus direction, a UBE2L3 polypeptide or a portion thereof fused to an ALK polypeptide or a portion thereof. In some embodiments, the ALK-UBE3B fusion polypeptide of the disclosure comprises, in the amino-terminus to carboxyl-terminus direction, a UBE3B polypeptide or a portion thereof fused to an ALK polypeptide or a portion thereof. In some embodiments, the ALK-UTRN fusion polypeptide of the disclosure comprises, in the amino-terminus to carboxyl-terminus direction, a UTRN polypeptide or a portion thereof fused to an ALK polypeptide or a portion thereof. In some embodiments, the ALK-VASP fusion polypeptide of the disclosure comprises, in the amino-terminus to carboxyl-terminus direction, a VASP polypeptide or a portion thereof fused to an ALK polypeptide or a portion thereof. In some embodiments, the ALK-WDR92 fusion polypeptide of the disclosure comprises, in the amino-terminus to carboxyl-terminus direction, a WDR92 polypeptide or a portion thereof fused to an ALK polypeptide or a portion thereof. In some embodiments, the ALK-YPEL5 fusion polypeptide of the disclosure comprises, in the amino-terminus to carboxyl-terminus direction, an YPEL5 polypeptide or a portion thereof fused to an ALK polypeptide or a portion thereof. In some embodiments, the ALK-ZNF446 fusion polypeptide of the disclosure comprises, in the amino-terminus to carboxyl-terminus direction, a ZNF446 polypeptide or a portion thereof fused to an ALK polypeptide or a portion thereof. In some embodiments, the ALK-ZNF454 fusion polypeptide of the disclosure comprises, in the amino-terminus to carboxyl-terminus direction, an ALK polypeptide or a portion thereof fused to a ZNF454 polypeptide or a portion thereof. In some embodiments, the ALK-ZNF513 fusion polypeptide of the disclosure comprises, in the amino-terminus to carboxyl-terminus direction, a ZNF513 polypeptide or a portion thereof fused to an ALK polypeptide or a portion thereof. In some embodiments, the ALK-ZSWIM2 fusion polypeptide of the disclosure comprises, in the amino-terminus to carboxyl-terminus direction, an ALK polypeptide or a portion thereof fused to a ZSWIM2 polypeptide or a portion thereof. In some embodiments, the ALK-FILIP1L fusion polypeptide of the disclosure comprises, in the amino-terminus to carboxyl-terminus direction, an ALK polypeptide or a portion thereof fused to an FILIP1L polypeptide or a portion thereof.

In some embodiments, the ALK-COL3A1 fusion polypeptide of the disclosure comprises a fusion of exon 48 of COL3A1, or a portion thereof, fused to exon 19 of ALK, or a portion thereof, e.g., in the amino-terminus to carboxyl-terminus direction. In some embodiments, the ALK-CDC42BPA fusion polypeptide of the disclosure comprises a fusion of exon 20 of CDC42BPA, or a portion thereof, fused to exon 20 of ALK, or a portion thereof, e.g., in the amino-terminus to carboxyl-terminus direction. In some embodiments, the ALK-EPHA2 fusion polypeptide of the disclosure comprises a fusion of exon 3 of EPHA2, or a portion thereof, fused to exon 19 of ALK, or a portion thereof, e.g., in the amino-terminus to carboxyl-terminus direction. In some embodiments, the ALK-MYO5C fusion polypeptide of the disclosure comprises a fusion of exon 30 of MYO5C, or a portion thereof, fused to exon 20 of ALK, or a portion thereof, e.g., in the amino-terminus to carboxyl-terminus direction. In some embodiments, the ALK-TRIM24 fusion polypeptide of the disclosure comprises a fusion of exon 12 of TRIM24, or a portion thereof, fused to exon 20 of ALK, or a portion thereof, e.g., in the amino-terminus to carboxyl-terminus direction. In some embodiments, the ALK-SKAP1 fusion polypeptide of the disclosure comprises a fusion of exon 3 of SKAP1, or a portion thereof, fused to exon 20 of ALK, or a portion thereof, e.g., in the amino-terminus to carboxyl-terminus direction. In some embodiments, the ALK-UBE3B fusion polypeptide of the disclosure comprises a fusion of exon 11 of UBE3B, or a portion thereof, fused to exon 20 of ALK, or a portion thereof, e.g., in the amino-terminus to carboxyl-terminus direction. In some embodiments, the ALK-TNS3 fusion polypeptide of the disclosure comprises a fusion of exon 25 of TNS3, or a portion thereof, fused to exon 20 of ALK, or a portion thereof, e.g., in the amino-terminus to carboxyl-terminus direction. In some embodiments, the ALK-C2orf73 fusion polypeptide of the disclosure comprises a fusion of exon 3 of C2orf73, or a portion thereof, fused to exon 20 of ALK, or a portion thereof, e.g., in the amino-terminus to carboxyl-terminus direction. In some embodiments, the ALK-AZI2 fusion polypeptide of the disclosure comprises a fusion of exon 7 of AZI2, or a portion thereof, fused to exon 20 of ALK, or a portion thereof, e.g., in the amino-terminus to carboxyl-terminus direction. In some embodiments, the ALK-MANBA fusion polypeptide of the disclosure comprises a fusion of exon 14 of MANBA, or a portion thereof, fused to exon 20 of ALK, or a portion thereof, e.g., in the amino-terminus to carboxyl-terminus direction. In some embodiments, the ALK-CNTNAP5 fusion polypeptide of the disclosure comprises a fusion of exon 2 of CNTNAP5, or a portion thereof, fused to exon 20 of ALK, or a portion thereof, e.g., in the amino-terminus to carboxyl-terminus direction. In some embodiments, the ALK-TANGO6 fusion polypeptide of the disclosure comprises a fusion of exon 1 of TANGO6, or a portion thereof, fused to exon 2 of ALK, or a portion thereof, e.g., in the amino-terminus to carboxyl-terminus direction. In some embodiments, the ALK-NFIA fusion polypeptide of the disclosure comprises a fusion of exon 2 of NFIA, or a portion thereof, fused to exon 2 of ALK, or a portion thereof, e.g., in the amino-terminus to carboxyl-terminus direction. In some embodiments, the ALK-RPS6KA5 fusion polypeptide of the disclosure comprises a fusion of exon 1 of RPS6KA5, or a portion thereof, fused to exon 8 of ALK, or a portion thereof, e.g., in the amino-terminus to carboxyl-terminus direction. In some embodiments, the ALK-TG fusion polypeptide of the disclosure comprises a fusion of exon 10 of TG, or a portion thereof, fused to exon 19 of ALK, or a portion thereof, e.g., in the amino-terminus to carboxyl-terminus direction. In some embodiments, the ALK-LRRFIP2 fusion polypeptide of the disclosure comprises a fusion of exon 25 of LRRFIP2, or a portion thereof, fused to exon 20 of ALK, or a portion thereof, e.g., in the amino-terminus to carboxyl-terminus direction. In some embodiments, the ALK-MYO5C fusion polypeptide of the disclosure comprises a fusion of exon 30 of MYO5C, or a portion thereof, fused to exon 20 of ALK, or a portion thereof, e.g., in the amino-terminus to carboxyl-terminus direction. In some embodiments, the ALK-AGAP1 fusion polypeptide of the disclosure comprises a fusion of exon 6 of AGAP1, or a portion thereof, fused to exon 18 of ALK, or a portion thereof, e.g., in the amino-terminus to carboxyl-terminus direction. In some embodiments, the ALK-MED13L fusion polypeptide of the disclosure comprises a fusion of exon 1 of ALK, or a portion thereof, fused to exon 6 of MED13L, or a portion thereof, e.g., in the amino-terminus to carboxyl-terminus direction. In some embodiments, the ALK-MTBP fusion polypeptide of the disclosure comprises a fusion of exon 17 of ALK, or a portion thereof, fused to exon 12 of MTBP, or a portion thereof, e.g., in the amino-terminus to carboxyl-terminus direction. In some embodiments, the ALK-SLC30A6 fusion polypeptide of the disclosure comprises a fusion of exon 18 of ALK, or a portion thereof, fused to exon 4 of SLC30A6, or a portion thereof, e.g., in the amino-terminus to carboxyl-terminus direction. In some embodiments, the ALK-GMCL1 fusion polypeptide of the disclosure comprises a fusion of exon 27 of ALK, or a portion thereof, fused to exon 7 of GMCL1, or a portion thereof, e.g., in the amino-terminus to carboxyl-terminus direction. In some embodiments, the ALK-AGAP1 fusion polypeptide of the disclosure comprises a fusion of exon 19 of ALK, or a portion thereof, fused to exon 7 of AGAP1, or a portion thereof, e.g., in the amino-terminus to carboxyl-terminus direction. In some embodiments, the ALK-ZNF454 fusion polypeptide of the disclosure comprises a fusion of exon 19 of ALK, or a portion thereof, fused to exon 5 of ZNF454, or a portion thereof, e.g., in the amino-terminus to carboxyl-terminus direction. In some embodiments, the ALK-TTC28 fusion polypeptide of the disclosure comprises a fusion of exon 1 of TTC28, or a portion thereof, fused to exon 14 of ALK, or a portion thereof, e.g., in the amino-terminus to carboxyl-terminus direction. In some embodiments, the ALK-NINJ2 fusion polypeptide of the disclosure comprises a fusion of exon 1 of NINJ2, or a portion thereof, fused to exon 14 of ALK, or a portion thereof, e.g., in the amino-terminus to carboxyl-terminus direction. In some embodiments, the ALK-UTRN fusion polypeptide of the disclosure comprises a fusion of exon 72 of UTRN, or a portion thereof, fused to exon 19 of ALK, or a portion thereof, e.g., in the amino-terminus to carboxyl-terminus direction. In some embodiments, the ALK-ACTN4 fusion polypeptide of the disclosure comprises a fusion of exon 7 of ACTN4, or a portion thereof, fused to exon 20 of ALK, or a portion thereof, e.g., in the amino-terminus to carboxyl-terminus direction. In some embodiments, the ALK-CPSF7 fusion polypeptide of the disclosure comprises a fusion of exon 5 of CPSF7, or a portion thereof, fused to exon 20 of ALK, or a portion thereof, e.g., in the amino-terminus to carboxyl-terminus direction. In some embodiments, the ALK-SLC25A13 fusion polypeptide of the disclosure comprises a fusion of exon 10 of SLC25A13, or a portion thereof, fused to exon 20 of ALK, or a portion thereof, e.g., in the amino-terminus to carboxyl-terminus direction. In some embodiments, the ALK-CTNND1 fusion polypeptide of the disclosure comprises a fusion of exon 16 of CTNND1, or a portion thereof, fused to exon 20 of ALK, or a portion thereof, e.g., in the amino-terminus to carboxyl-terminus direction. In some embodiments, the ALK-KLC4 fusion polypeptide of the disclosure comprises a fusion of exon 9 of KLC4, or a portion thereof, fused to exon 20 of ALK, or a portion thereof, e.g., in the amino-terminus to carboxyl-terminus direction. In some embodiments, the ALK-BTBD9 fusion polypeptide of the disclosure comprises a fusion of exon 5 of BTBD9, or a portion thereof, fused to exon 20 of ALK, or a portion thereof, e.g., in the amino-terminus to carboxyl-terminus direction. In some embodiments, the ALK-CPQ fusion polypeptide of the disclosure comprises a fusion of exon 7 of CPQ, or a portion thereof, fused to exon 20 of ALK, or a portion thereof, e.g., in the amino-terminus to carboxyl-terminus direction. In some embodiments, the ALK-KIF5C fusion polypeptide of the disclosure comprises a fusion of exon 11 of KIF5C, or a portion thereof, fused to exon 20 of ALK, or a portion thereof, e.g., in the amino-terminus to carboxyl-terminus direction. In some embodiments, the ALK-MAGOHB fusion polypeptide of the disclosure comprises a fusion of exon 1 of MAGOHB, or a portion thereof, fused to exon 20 of ALK, or a portion thereof, e.g., in the amino-terminus to carboxyl-terminus direction. In some embodiments, the ALK-COL3A1 fusion polypeptide of the disclosure comprises a fusion of exon 48 of COL3A1, or a portion thereof, fused to exon 19 of ALK, or a portion thereof, e.g., in the amino-terminus to carboxyl-terminus direction. In some embodiments, the ALK-OPRM1 fusion polypeptide of the disclosure comprises a fusion of exon 3 of OPRM1, or a portion thereof, fused to exon 20 of ALK, or a portion thereof, e.g., in the amino-terminus to carboxyl-terminus direction. In some embodiments, the ALK-GPN1 fusion polypeptide of the disclosure comprises a fusion of exon 1 of GPN1, or a portion thereof, fused to exon 16 of ALK, or a portion thereof, e.g., in the amino-terminus to carboxyl-terminus direction. In some embodiments, the ALK-SEC16B fusion polypeptide of the disclosure comprises a fusion of exon 13 of SEC16B, or a portion thereof, fused to exon 14 of ALK, or a portion thereof, e.g., in the amino-terminus to carboxyl-terminus direction. In some embodiments, the ALK-UBE2L3 fusion polypeptide of the disclosure comprises a fusion of exon 1 of UBE2L3, or a portion thereof, fused to exon 18 of ALK, or a portion thereof, e.g., in the amino-terminus to carboxyl-terminus direction. In some embodiments, the ALK-METTL25 fusion polypeptide of the disclosure comprises a fusion of exon 8 of METTL25, or a portion thereof, fused to exon 20 of ALK, or a portion thereof, e.g., in the amino-terminus to carboxyl-terminus direction. In some embodiments, the ALK-CYS1 fusion polypeptide of the disclosure comprises a fusion of exon 1 of CYS1, or a portion thereof, fused to exon 20 of ALK, or a portion thereof, e.g., in the amino-terminus to carboxyl-terminus direction. In some embodiments, the ALK-ABCB11 fusion polypeptide of the disclosure comprises a fusion of exon 26 of ABCB11, or a portion thereof, fused to exon 20 of ALK, or a portion thereof, e.g., in the amino-terminus to carboxyl-terminus direction. In some embodiments, the ALK-INTS9 fusion polypeptide of the disclosure comprises a fusion of exon 8 of INTS9, or a portion thereof, fused to exon 20 of ALK, or a portion thereof, e.g., in the amino-terminus to carboxyl-terminus direction. In some embodiments, the ALK-CIB4 fusion polypeptide of the disclosure comprises a fusion of exon 3 of CIB4, or a portion thereof, fused to exon 20 of ALK, or a portion thereof, e.g., in the amino-terminus to carboxyl-terminus direction. In some embodiments, the ALK-WDR92 fusion polypeptide of the disclosure comprises a fusion of exon 7 of WDR92, or a portion thereof, fused to exon 2 of ALK, or a portion thereof, e.g., in the amino-terminus to carboxyl-terminus direction. In some embodiments, the ALK-OTX1 fusion polypeptide of the disclosure comprises a fusion of exon 19 of ALK, or a portion thereof, fused to exon 5 of OTX1, or a portion thereof, e.g., in the amino-terminus to carboxyl-terminus direction. In some embodiments, the ALK-PDCD10 fusion polypeptide of the disclosure comprises a fusion of exon 15 of ALK, or a portion thereof, fused to exon 3 of PDCD10, or a portion thereof, e.g., in the amino-terminus to carboxyl-terminus direction. In some embodiments, the ALK-PTGER4 fusion polypeptide of the disclosure comprises a fusion of exon 19 of ALK, or a portion thereof, fused to exon 3 of PTGER4, or a portion thereof, e.g., in the amino-terminus to carboxyl-terminus direction. In some embodiments, the ALK-PTPRJ fusion polypeptide of the disclosure comprises a fusion of exon 19 of ALK, or a portion thereof, fused to exon 2 of PTPRJ, or a portion thereof, e.g., in the amino-terminus to carboxyl-terminus direction. In some embodiments, the ALK-ZSWIM2 fusion polypeptide of the disclosure comprises a fusion of exon 19 of ALK, or a portion thereof, fused to exon 6 of ZSWIM2, or a portion thereof, e.g., in the amino-terminus to carboxyl-terminus direction. In some embodiments, the ALK-FHOD3 fusion polypeptide of the disclosure comprises a fusion of exon 18 of ALK, or a portion thereof, fused to exon 10 of FHOD3, or a portion thereof, e.g., in the amino-terminus to carboxyl-terminus direction. In some embodiments, the ALK-FILIP1L fusion polypeptide of the disclosure comprises a fusion of exon 18 of ALK, or a portion thereof, fused to exon 2 of FILP1L, or a portion thereof, e.g., in the amino-terminus to carboxyl-terminus direction. In some embodiments, the ALK-ITGA6 fusion polypeptide of the disclosure comprises a fusion of exon 19 of ALK, or a portion thereof, fused to exon 2 of ITGA6, or a portion thereof, e.g., in the amino-terminus to carboxyl-terminus direction. In some embodiments, the ALK-KCTD18 fusion polypeptide of the disclosure comprises a fusion of exon 19 of ALK, or a portion thereof, fused to exon 2 of KCTD18, or a portion thereof, e.g., in the amino-terminus to carboxyl-terminus direction. In some embodiments, the ALK-MAMDC4 fusion polypeptide of the disclosure comprises a fusion of exon 20 of ALK, or a portion thereof, fused to exon 16 of MAMDC4, or a portion thereof, e.g., in the amino-terminus to carboxyl-terminus direction. In some embodiments, the ALK-PELI1 fusion polypeptide of the disclosure comprises exon 20 of ALK, or a portion thereof, e.g., in the amino-terminus to carboxyl-terminus direction. In some embodiments, the ALK-LINC00535 fusion polypeptide of the disclosure comprises exon 20 of ALK, or a portion thereof, e.g., in the amino-terminus to carboxyl-terminus direction. In some embodiments, the ALK-CTBP1 fusion polypeptide of the disclosure comprises a fusion of exon 9 of CTBP1, or a portion thereof, fused to exon 20 of ALK, or a portion thereof, e.g., in the amino-terminus to carboxyl-terminus direction. In some embodiments, the ALK-CARMIL1 fusion polypeptide of the disclosure comprises a fusion of exon 27 of CARMIL1, or a portion thereof, fused to exon 20 of ALK, or a portion thereof, e.g., in the amino-terminus to carboxyl-terminus direction. In some embodiments, the ALK-ZNF513 fusion polypeptide of the disclosure comprises a fusion of exon 1 of ZNF513, or a portion thereof, fused to exon 1 of ALK, or a portion thereof, e.g., in the amino-terminus to carboxyl-terminus direction. In some embodiments, the ALK-TMCO3 fusion polypeptide of the disclosure comprises a fusion of exon 6 of TMCO3, or a portion thereof, fused to exon 11 of ALK, or a portion thereof, e.g., in the amino-terminus to carboxyl-terminus direction. In some embodiments, the ALK-SRSF7 fusion polypeptide of the disclosure comprises a fusion of exon 4 of SRSF7, or a portion thereof, fused to exon 18 of ALK, or a portion thereof, e.g., in the amino-terminus to carboxyl-terminus direction. In some embodiments, the ALK-CASP8 fusion polypeptide of the disclosure comprises a fusion of exon 10 of CASP8, or a portion thereof, fused to exon 2 of ALK, or a portion thereof, e.g., in the amino-terminus to carboxyl-terminus direction. In some embodiments, the ALK-CYP51A1 fusion polypeptide of the disclosure comprises a fusion of exon 9 of CYP51A1, or a portion thereof, fused to exon 3 of ALK, or a portion thereof, e.g., in the amino-terminus to carboxyl-terminus direction. In some embodiments, the ALK-GPR113 fusion polypeptide of the disclosure comprises a fusion of exon 1 of GPR113, or a portion thereof, fused to exon 19 of ALK, or a portion thereof, e.g., in the amino-terminus to carboxyl-terminus direction. In some embodiments, the ALK-HADHA fusion polypeptide of the disclosure comprises a fusion of exon 5 of HADHA, or a portion thereof, fused to exon 5 of ALK, or a portion thereof, e.g., in the amino-terminus to carboxyl-terminus direction. In some embodiments, the ALK-LRRFIP2 fusion polypeptide of the disclosure comprises a fusion of exon 22 of LRRFIP2, or a portion thereof, fused to exon 20 of ALK, or a portion thereof, e.g., in the amino-terminus to carboxyl-terminus direction. In some embodiments, the ALK-MYH10 fusion polypeptide of the disclosure comprises a fusion of exon 38 of MYH10, or a portion thereof, fused to exon 20 of ALK, or a portion thereof, e.g., in the amino-terminus to carboxyl-terminus direction. In some embodiments, the ALK-PDE3A fusion polypeptide of the disclosure comprises a fusion of exon 10 of PDE3A, or a portion thereof, fused to exon 8 of ALK, or a portion thereof, e.g., in the amino-terminus to carboxyl-terminus direction. In some embodiments, the ALK-PLEC fusion polypeptide of the disclosure comprises a fusion of exon 9 of PLEC, or a portion thereof, fused to exon 20 of ALK, or a portion thereof, e.g., in the amino-terminus to carboxyl-terminus direction. In some embodiments, the ALK-QKI fusion polypeptide of the disclosure comprises a fusion of exon 2 of QKI, or a portion thereof, fused to exon 20 of ALK, or a portion thereof, e.g., in the amino-terminus to carboxyl-terminus direction. In some embodiments, the ALK-SASH1 fusion polypeptide of the disclosure comprises a fusion of exon 14 of SASH1, or a portion thereof, fused to exon 18 of ALK, or a portion thereof, e.g., in the amino-terminus to carboxyl-terminus direction. In some embodiments, the ALK-SRSF7 fusion polypeptide of the disclosure comprises a fusion of exon 5 of SRSF7, or a portion thereof, fused to exon 18 of ALK, or a portion thereof, e.g., in the amino-terminus to carboxyl-terminus direction. In some embodiments, the ALK-VASP fusion polypeptide of the disclosure comprises a fusion of exon 4 of VASP, or a portion thereof, fused to exon 7 of ALK, or a portion thereof, e.g., in the amino-terminus to carboxyl-terminus direction. In some embodiments, the ALK-ZNF446 fusion polypeptide of the disclosure comprises a fusion of exon 7 of ZNF446, or a portion thereof, fused to exon 20 of ALK, or a portion thereof, e.g., in the amino-terminus to carboxyl-terminus direction. In some embodiments, the ALK-SOX13 fusion polypeptide of the disclosure comprises exon 18 of ALK, or a portion thereof, e.g., in the amino-terminus to carboxyl-terminus direction. In some embodiments, the ALK-YPEL5 fusion polypeptide of the disclosure comprises exon 18 of ALK, or a portion thereof, e.g., in the amino-terminus to carboxyl-terminus direction. In some embodiments, the ALK-CAPN14 fusion polypeptide of the disclosure comprises a fusion of exon 14 of ALK, or a portion thereof, fused to exon 3 of CAPN14, or a portion thereof, e.g., in the amino-terminus to carboxyl-terminus direction. In some embodiments, the ALK-MAP3K9 fusion polypeptide of the disclosure comprises a fusion of exon 1 of ALK, or a portion thereof, fused to exon 4 of MAP3K9, or a portion thereof, e.g., in the amino-terminus to carboxyl-terminus direction. In some embodiments, the ALK-SNX17 fusion polypeptide of the disclosure comprises a fusion of exon 13 of ALK, or a portion thereof, fused to exon 3 of SNX17, or a portion thereof, e.g., in the amino-terminus to carboxyl-terminus direction. In some embodiments, the ALK-HS1BP3 fusion polypeptide of the disclosure comprises a fusion of exon 19 of ALK, or a portion thereof, fused to exon 4 of HS1BP3, or a portion thereof, e.g., in the amino-terminus to carboxyl-terminus direction. In some embodiments, the ALK-CREBBP fusion polypeptide of the disclosure comprises a fusion of exon 3 of ALK, or a portion thereof, fused to exon 2 of CREBBP, or a portion thereof, e.g., in the amino-terminus to carboxyl-terminus direction. In some embodiments, the ALK-PAQR4 fusion polypeptide of the disclosure comprises a fusion of exon 3 of ALK, or a portion thereof, fused to exon 1 of PAQR4, or a portion thereof, e.g., in the amino-terminus to carboxyl-terminus direction. In some embodiments, the ALK-APH1A fusion polypeptide of the disclosure comprises a fusion of exon 4 of ALK, or a portion thereof, fused to exon 4 of APH1A, or a portion thereof, e.g., in the amino-terminus to carboxyl-terminus direction.

In some embodiments, the ALK-COL3A1 fusion polypeptide of the disclosure comprises exons 1-47, and exon 48 or a portion thereof, of COL3A1 fused to exon 19 or a portion thereof, and exons 20-29, of ALK, e.g., in the amino-terminus to carboxyl-terminus direction. In some embodiments, the ALK-CDC42BPA fusion polypeptide of the disclosure comprises exons 1-19, and exon 20 or a portion thereof, of CDC42BPA fused to exon 20 or a portion thereof, and exons 21-29, of ALK, e.g., in the amino-terminus to carboxyl-terminus direction. In some embodiments, the ALK-EPHA2 fusion polypeptide of the disclosure comprises exons 1-2, and exon 3 or a portion thereof, of EPHA2 fused to exon 19 or a portion thereof, and exons 20-29, of ALK, e.g., in the amino-terminus to carboxyl-terminus direction. In some embodiments, the ALK-MYO5C fusion polypeptide of the disclosure comprises exons 1-29, and exon 30 or a portion thereof, of MYO5C fused to exon 20 or a portion thereof, and exons 21-29, of ALK, e.g., in the amino-terminus to carboxyl-terminus direction. In some embodiments, the ALK-TRIM24 fusion polypeptide of the disclosure comprises exons 1-11, and exon 12 or a portion thereof, of TRIM24 fused to exon 20 or a portion thereof, and exons 21-29, of ALK, e.g., in the amino-terminus to carboxyl-terminus direction. In some embodiments, the ALK-SKAP1 fusion polypeptide of the disclosure comprises exons 1-2, and exon 3 or a portion thereof, of SKAP1 fused to exon 20 or a portion thereof, and exons 21-29, of ALK, e.g., in the amino-terminus to carboxyl-terminus direction. In some embodiments, the ALK-UBE3B fusion polypeptide of the disclosure comprises exons 1-10, and exon 11 or a portion thereof, of UBE3B fused to exon 20 or a portion thereof, and exons 21-29, of ALK, e.g., in the amino-terminus to carboxyl-terminus direction. In some embodiments, the ALK-TNS3 fusion polypeptide of the disclosure comprises exons 1-24, and exon 25 or a portion thereof, of TNS3 fused to exon 20 or a portion thereof, and exons 21-29, of ALK, e.g., in the amino-terminus to carboxyl-terminus direction. In some embodiments, the ALK-C2orf73 fusion polypeptide of the disclosure comprises exons 1-2, and exon 3 or a portion thereof, of C2orf73 fused to exon 20 or a portion thereof, and exons 21-29, of ALK, e.g., in the amino-terminus to carboxyl-terminus direction. In some embodiments, the ALK-AZI2 fusion polypeptide of the disclosure comprises exons 1-6, and exon 7 or a portion thereof, of AZI2 fused to exon 20 or a portion thereof, and exons 21-29, of ALK, e.g., in the amino-terminus to carboxyl-terminus direction. In some embodiments, the ALK-MANBA fusion polypeptide of the disclosure comprises exons 1-13, and exon 14 or a portion thereof, of MANBA fused to exon 20 or a portion thereof, and exons 21-29, of ALK, e.g., in the amino-terminus to carboxyl-terminus direction. In some embodiments, the ALK-CNTNAP5 fusion polypeptide of the disclosure comprises exon 1, and exon 2 or a portion thereof, of CNTNAP5 fused to exon 20 or a portion thereof, and exons 21-29, of ALK, e.g., in the amino-terminus to carboxyl-terminus direction. In some embodiments, the ALK-TANGO6 fusion polypeptide of the disclosure comprises exon 1 or a portion thereof of TANGO6 fused to exon 2 or a portion thereof, and exons 3-29, of ALK, e.g., in the amino-terminus to carboxyl-terminus direction. In some embodiments, the ALK-NFIA fusion polypeptide of the disclosure comprises exon 1, and exon 2 or a portion thereof, of NFIA fused to exon 2 or a portion thereof, and exons 3-29, of ALK, e.g., in the amino-terminus to carboxyl-terminus direction. In some embodiments, the ALK-RPS6KA5 fusion polypeptide of the disclosure comprises exon 1 or a portion thereof of RPS6KA5 fused to exon 8 or a portion thereof, and exons 9-29, of ALK, e.g., in the amino-terminus to carboxyl-terminus direction. In some embodiments, the ALK-TG fusion polypeptide of the disclosure comprises exons 1-9, and exon 10 or a portion thereof, of TG fused to exon 19 or a portion thereof, and exons 20-29, of ALK, e.g., in the amino-terminus to carboxyl-terminus direction. In some embodiments, the ALK-LRRFIP2 fusion polypeptide of the disclosure comprises exons 1-24, and exon 25 or a portion thereof, of LRRFIP2 fused to exon 20 or a portion thereof, and exons 21-29, of ALK, e.g., in the amino-terminus to carboxyl-terminus direction. In some embodiments, the ALK-MYO5C fusion polypeptide of the disclosure comprises exons 1-29, and exon 30 or a portion thereof, of MYO5C fused to exon 20 or a portion thereof, and exons 21-29, of ALK, e.g., in the amino-terminus to carboxyl-terminus direction. In some embodiments, the ALK-AGAP1 fusion polypeptide of the disclosure comprises exons 1-5, and exon 6 or a portion thereof, of AGAP1 fused to exon 18 or a portion thereof, and exons 19-29, of ALK, e.g., in the amino-terminus to carboxyl-terminus direction. In some embodiments, the ALK-MED13L fusion polypeptide of the disclosure comprises exon 1 or a portion thereof of ALK fused to exon 6 or a portion thereof, and exons 7-31, of MED13L, e.g., in the amino-terminus to carboxyl-terminus direction. In some embodiments, the ALK-MTBP fusion polypeptide of the disclosure comprises exons 1-16, and exon 17 or a portion thereof, of ALK fused to exon 12 or a portion thereof, and exons 13-22, of MTBP, e.g., in the amino-terminus to carboxyl-terminus direction. In some embodiments, the ALK-SLC30A6 fusion polypeptide of the disclosure comprises exons 1-17, and exon 18 or a portion thereof, of ALK fused to exon 4 or a portion thereof, and exons 5-14, of SLC30A6, e.g., in the amino-terminus to carboxyl-terminus direction. In some embodiments, the ALK-GMCL1 fusion polypeptide of the disclosure comprises exons 1-26, and exon 27 or a portion thereof, of ALK fused to exon 7 or a portion thereof, and exons 8-14, of GMCL1, e.g., in the amino-terminus to carboxyl-terminus direction. In some embodiments, the ALK-AGAP1 fusion polypeptide of the disclosure comprises exons 1-18, and exon 19 or a portion thereof, of ALK fused to exon 7 or a portion thereof, and exons 8-17, of AGAP1, e.g., in the amino-terminus to carboxyl-terminus direction. In some embodiments, the ALK-ZNF454 fusion polypeptide of the disclosure comprises exons 1-18, and exon 19 or a portion thereof, of ALK fused to exon 5 or a portion thereof of ZNF454, e.g., in the amino-terminus to carboxyl-terminus direction. In some embodiments, the ALK-TTC28 fusion polypeptide of the disclosure comprises exon 1 or a portion thereof of TTC28 fused to exon 14 or a portion thereof, and exons 15-29, of ALK, e.g., in the amino-terminus to carboxyl-terminus direction. In some embodiments, the ALK-NINJ2 fusion polypeptide of the disclosure comprises exon 1 or a portion thereof of NINJ2 fused to exon 14 or a portion thereof, and exons 15-29, of ALK, e.g., in the amino-terminus to carboxyl-terminus direction. In some embodiments, the ALK-UTRN fusion polypeptide of the disclosure comprises exons 1-71, and exon 72 or a portion thereof, of UTRN fused to exon 19 or a portion thereof, and exons 20-29, of ALK, e.g., in the amino-terminus to carboxyl-terminus direction. In some embodiments, the ALK-ACTN4 fusion polypeptide of the disclosure comprises exons 1-6, and exon 7 or a portion thereof, of ACTN4 fused to exon 20 or a portion thereof, and exons 21-29, of ALK, e.g., in the amino-terminus to carboxyl-terminus direction. In some embodiments, the ALK-CPSF7 fusion polypeptide of the disclosure comprises exons 1-4, and exon 5 or a portion thereof, of CPSF7 fused to exon 20 or a portion thereof, and exons 21-29, of ALK, e.g., in the amino-terminus to carboxyl-terminus direction. In some embodiments, the ALK-SLC25A13 fusion polypeptide of the disclosure comprises exons 1-9, and exon 10 or a portion thereof, of SLC25A13 fused to exon 20 or a portion thereof, and exons 21-29, of ALK, e.g., in the amino-terminus to carboxyl-terminus direction. In some embodiments, the ALK-CTNND1 fusion polypeptide of the disclosure comprises exons 1-15, and exon 16 or a portion thereof, of CTNND1 fused to exon 20 or a portion thereof, and exons 21-29, of ALK, e.g., in the amino-terminus to carboxyl-terminus direction. In some embodiments, the ALK-KLC4 fusion polypeptide of the disclosure comprises exons 1-8, and exon 9 or a portion thereof, of KLC4 fused to exon 20 or a portion thereof, and exons 21-29, of ALK, e.g., in the amino-terminus to carboxyl-terminus direction. In some embodiments, the ALK-BTBD9 fusion polypeptide of the disclosure comprises exons 1-4, and exon 5 or a portion thereof, of BTBD9 fused to exon 20 or a portion thereof, and exons 21-29, of ALK, e.g., in the amino-terminus to carboxyl-terminus direction. In some embodiments, the ALK-CPQ fusion polypeptide of the disclosure comprises exons 1-6, and exon 7 or a portion thereof, of CPQ fused to exon 20 or a portion thereof, and exons 21-29, of ALK, e.g., in the amino-terminus to carboxyl-terminus direction. In some embodiments, the ALK-KIF5C fusion polypeptide of the disclosure comprises exons 1-10, and exon 11 or a portion thereof, of KIF5C fused to exon 20 or a portion thereof, and exons 21-29, of ALK, e.g., in the amino-terminus to carboxyl-terminus direction. In some embodiments, the ALK-MAGOHB fusion polypeptide of the disclosure comprises exon 1 or a portion thereof of MAGOHB fused to exon 20 or a portion thereof, and exons 21-29, of ALK, e.g., in the amino-terminus to carboxyl-terminus direction. In some embodiments, the ALK-COL3A1 fusion polypeptide of the disclosure comprises exons 1-47, and exon 48 or a portion thereof, of COL3A1 fused to exon 19 or a portion thereof, and exons 20-29, of ALK, e.g., in the amino-terminus to carboxyl-terminus direction. In some embodiments, the ALK-OPRM1 fusion polypeptide of the disclosure comprises exons 1-2, and exon 3 or a portion thereof, of OPRM1 fused to exon 20 or a portion thereof, and exons 21-29, of ALK, e.g., in the amino-terminus to carboxyl-terminus direction. In some embodiments, the ALK-GPN1 fusion polypeptide of the disclosure comprises exon 1 or a portion thereof of GPN1 fused to exon 16 or a portion thereof, and exons 17-29, of ALK, e.g., in the amino-terminus to carboxyl-terminus direction. In some embodiments, the ALK-SEC16B fusion polypeptide of the disclosure comprises exons 1-12, and exon 13 or a portion thereof, of SEC16B fused to exon 14 or a portion thereof, and exons 15-29, of ALK, e.g., in the amino-terminus to carboxyl-terminus direction. In some embodiments, the ALK-UBE2L3 fusion polypeptide of the disclosure comprises exon 1 or a portion thereof of UBE2L3 fused to exon 18 or a portion thereof, and exons 19-29, of ALK, e.g., in the amino-terminus to carboxyl-terminus direction. In some embodiments, the ALK-METTL25 fusion polypeptide of the disclosure comprises exons 1-7, and exon 8 or a portion thereof, of METTL25 fused to exon 20 or a portion thereof, and exons 21-29, of ALK, e.g., in the amino-terminus to carboxyl-terminus direction. In some embodiments, the ALK-CYS1 fusion polypeptide of the disclosure comprises exon 1 or a portion thereof of CYS1 fused to exon 20 or a portion thereof, and exons 21-29, of ALK, e.g., in the amino-terminus to carboxyl-terminus direction. In some embodiments, the ALK-ABCB11 fusion polypeptide of the disclosure comprises exons 1-25, and exon 26 or a portion thereof, of ABCB11 fused to exon 20 or a portion thereof, and exons 21-29, of ALK, e.g., in the amino-terminus to carboxyl-terminus direction. In some embodiments, the ALK-INTS9 fusion polypeptide of the disclosure comprises exons 1-7, and exon 8 or a portion thereof, of INTS9 fused to exon 20 or a portion thereof, and exons 21-29, of ALK, e.g., in the amino-terminus to carboxyl-terminus direction. In some embodiments, the ALK-CIB4 fusion polypeptide of the disclosure comprises exons 1-2, and exon 3 or a portion thereof, of CIB4 fused to exon 20 or a portion thereof, and exons 21-29, of ALK, e.g., in the amino-terminus to carboxyl-terminus direction. In some embodiments, the ALK-WDR92 fusion polypeptide of the disclosure comprises exons 1-6, and exon 7 or a portion thereof, of WDR92 fused to exon 2 or a portion thereof, and exons 3-29 of ALK, e.g., in the amino-terminus to carboxyl-terminus direction. In some embodiments, the ALK-OTX1 fusion polypeptide of the disclosure comprises exons 1-18, and exon 19 or a portion thereof, of ALK fused to exon 5 or a portion thereof of OTX1, e.g., in the amino-terminus to carboxyl-terminus direction. In some embodiments, the ALK-PDCD10 fusion polypeptide of the disclosure comprises exons 1-14, and exon 15 or a portion thereof, of ALK fused to exon 3 or a portion thereof, and exons 4-9, of PDCD10, e.g., in the amino-terminus to carboxyl-terminus direction. In some embodiments, the ALK-PTGER4 fusion polypeptide of the disclosure comprises exons 1-18, and exon 19 or a portion thereof, of ALK fused to exon 3 or a portion thereof of PTGER4, e.g., in the amino-terminus to carboxyl-terminus direction. In some embodiments, the ALK-PTPRJ fusion polypeptide of the disclosure comprises exons 1-18, and exon 19 or a portion thereof, of ALK fused to exon 2 or a portion thereof, and exons 3-25, of PTPRJ, e.g., in the amino-terminus to carboxyl-terminus direction. In some embodiments, the ALK-ZSWIM2 fusion polypeptide of the disclosure comprises exons 1-18, and exon 19 or a portion thereof, of ALK fused to exon 6 or a portion thereof, and exons 7-9, of ZSWIM2, e.g., in the amino-terminus to carboxyl-terminus direction. In some embodiments, the ALK-FHOD3 fusion polypeptide of the disclosure comprises exons 1-17, and exon 18 or a portion thereof, of ALK fused to exon 10 or a portion thereof, and exons 11-25, of FHOD3, e.g., in the amino-terminus to carboxyl-terminus direction. In some embodiments, the ALK-FILIP1L fusion polypeptide of the disclosure comprises exons 1-17, and exon 18 or a portion thereof, of ALK fused to exon 2 or a portion thereof, and exons 3-6, of FILP1L, e.g., in the amino-terminus to carboxyl-terminus direction. In some embodiments, the ALK-ITGA6 fusion polypeptide of the disclosure comprises exons 1-18, and exon 19 or a portion thereof, of ALK fused to exon 2 or a portion thereof, and exons 3-26, of ITGA6, e.g., in the amino-terminus to carboxyl-terminus direction. In some embodiments, the ALK-KCTD18 fusion polypeptide of the disclosure comprises exons 1-18, and exon 19 or a portion thereof, of ALK fused to exon 2 or a portion thereof, and exons 3-7, of KCTD18, e.g., in the amino-terminus to carboxyl-terminus direction. In some embodiments, the ALK-MAMDC4 fusion polypeptide of the disclosure comprises exons 1-19, and exon 20 or a portion thereof, of ALK fused to exon 16 or a portion thereof, and exons 17-27, of MAMDC4, e.g., in the amino-terminus to carboxyl-terminus direction. In some embodiments, the ALK-PELI1 fusion polypeptide of the disclosure comprises exon 20 or a portion thereof, and exons 21-29, of ALK, e.g., in the amino-terminus to carboxyl-terminus direction. In some embodiments, the ALK-LINC00535 fusion polypeptide of the disclosure comprises exon 20 or a portion thereof, and exons 21-29, of ALK, e.g., in the amino-terminus to carboxyl-terminus direction. In some embodiments, the ALK-CTBP1 fusion polypeptide of the disclosure comprises exons 1-8, and exon 9 or a portion thereof, of CTBP1 fused to exon 20 or a portion thereof, and exons 21-29 of ALK, e.g., in the amino-terminus to carboxyl-terminus direction. In some embodiments, the ALK-CARMIL1 fusion polypeptide of the disclosure comprises exons 1-26, and exon 27 or a portion thereof, of CARMIL1 fused to exon 20 or a portion thereof, and exons 21-29 of ALK, e.g., in the amino-terminus to carboxyl-terminus direction. In some embodiments, the ALK-ZNF513 fusion polypeptide of the disclosure comprises exon 1 or a portion thereof of ZNF513 fused to exon 1 or a portion thereof, and exons 2-29, of ALK, e.g., in the amino-terminus to carboxyl-terminus direction. In some embodiments, the ALK-TMCO3 fusion polypeptide of the disclosure comprises exons 1-5, and exon 6 or a portion thereof, of TMCO3 fused to exon 11 or a portion thereof, and exons 12-29, of ALK, e.g., in the amino-terminus to carboxyl-terminus direction. In some embodiments, the ALK-SRSF7 fusion polypeptide of the disclosure comprises exons 1-3, and exon 4 or a portion thereof, of SRSF7 fused to exon 18 or a portion thereof, and exons 19-29, of ALK, e.g., in the amino-terminus to carboxyl-terminus direction. In some embodiments, the ALK-CASP8 fusion polypeptide of the disclosure comprises exons 1-9, and exon 10 or a portion thereof, of CASP8 fused to exon 2 or a portion thereof, and exons 3-29, of ALK, e.g., in the amino-terminus to carboxyl-terminus direction. In some embodiments, the ALK-CYP51A1 fusion polypeptide of the disclosure comprises exons 1-8, and exon 9 or a portion thereof, of CYP51A1 fused to exon 3 or a portion thereof, and exons 4-29, of ALK, e.g., in the amino-terminus to carboxyl-terminus direction. In some embodiments, the ALK-GPR113 fusion polypeptide of the disclosure comprises exon 1 or a portion thereof of GPR113 fused to exon 19 or a portion thereof, and exons 20-29 of ALK, e.g., in the amino-terminus to carboxyl-terminus direction. In some embodiments, the ALK-HADHA fusion polypeptide of the disclosure comprises exons 1-4, and exon 5 or a portion thereof, of HADHA fused to exon 5 or a portion thereof, and exons 6-29, of ALK, e.g., in the amino-terminus to carboxyl-terminus direction. In some embodiments, the ALK-LRRFIP2 fusion polypeptide of the disclosure comprises exons 1-21, and exon 22 or a portion thereof, of LRRFIP2 fused to exon 20 or a portion thereof, and exons 21-29 of ALK, e.g., in the amino-terminus to carboxyl-terminus direction. In some embodiments, the ALK-MYH10 fusion polypeptide of the disclosure comprises exons 1-37, and exon 38 or a portion thereof, of MYH10 fused to exon 20 or a portion thereof, and exons 21-29 of ALK, e.g., in the amino-terminus to carboxyl-terminus direction. In some embodiments, the ALK-PDE3A fusion polypeptide of the disclosure comprises exons 1-9, and exon 10 or a portion thereof, of PDE3A fused to exon 8 or a portion thereof, and exons 9-29, of ALK, e.g., in the amino-terminus to carboxyl-terminus direction. In some embodiments, the ALK-PLEC fusion polypeptide of the disclosure comprises exons 1-8, and exon 9 or a portion thereof, of PLEC fused to exon 20 or a portion thereof, and exons 21-29 of ALK, e.g., in the amino-terminus to carboxyl-terminus direction. In some embodiments, the ALK-QKI fusion polypeptide of the disclosure comprises exon 1, and exon 2 or a portion thereof, of QKI fused to exon 20 or a portion thereof, and exons 21-29 of ALK, e.g., in the amino-terminus to carboxyl-terminus direction. In some embodiments, the ALK-SASH1 fusion polypeptide of the disclosure comprises exons 1-13, and exon 14 or a portion thereof, of SASH1 fused to exon 18 or a portion thereof, and exons 19-29, of ALK, e.g., in the amino-terminus to carboxyl-terminus direction. In some embodiments, the ALK-SRSF7 fusion polypeptide of the disclosure comprises exons 1-4, and exon 5 or a portion thereof, of SRSF7 fused to exon 18 or a portion thereof, and exons 19-29, of ALK, e.g., in the amino-terminus to carboxyl-terminus direction. In some embodiments, the ALK-VASP fusion polypeptide of the disclosure comprises exons 1-3, and exon 4 or a portion thereof, of VASP fused to exon 7 or a portion thereof, and exons 8-29, of ALK, e.g., in the amino-terminus to carboxyl-terminus direction. In some embodiments, the ALK-ZNF446 fusion polypeptide of the disclosure comprises exons 1-6, and exon 7 or a portion thereof, of ZNF446 fused to exon 20 or a portion thereof, and exons 21-29 of ALK, e.g., in the amino-terminus to carboxyl-terminus direction. In some embodiments, the ALK-SOX13 fusion polypeptide of the disclosure comprises exon 18 or a portion thereof, and exons 19-29, of ALK, e.g., in the amino-terminus to carboxyl-terminus direction. In some embodiments, the ALK-YPEL5 fusion polypeptide of the disclosure comprises exon 18 or a portion thereof, and exons 19-29, of ALK, e.g., in the amino-terminus to carboxyl-terminus direction. In some embodiments, the ALK-CAPN14 fusion polypeptide of the disclosure comprises exons 1-13, and exon 14 or a portion thereof, of ALK fused to exon 3 or a portion thereof, and exons 4-22, of CAPN14, e.g., in the amino-terminus to carboxyl-terminus direction. In some embodiments, the ALK-MAP3K9 fusion polypeptide of the disclosure comprises exon 1 or a portion thereof of ALK fused to exon 4 or a portion thereof, and exons 5-13, of MAP3K9, e.g., in the amino-terminus to carboxyl-terminus direction. In some embodiments, the ALK-SNX17 fusion polypeptide of the disclosure comprises exons 1-12, and exon 13 or a portion thereof, of ALK fused to exon 3 or a portion thereof, and exons 4-15, of SNX17, e.g., in the amino-terminus to carboxyl-terminus direction. In some embodiments, the ALK-HSmBP3 fusion polypeptide of the disclosure comprises exons 1-18, and exon 19 or a portion thereof, of ALK fused to exon 4 or a portion thereof, and exons 5-7, of HS1BP3, e.g., in the amino-terminus to carboxyl-terminus direction. In some embodiments, the ALK-CREBBP fusion polypeptide of the disclosure comprises exons 1-2, and exon 3 or a portion thereof, of ALK fused to exon 2 or a portion thereof, and exons 3-31, of CREBBP, e.g., in the amino-terminus to carboxyl-terminus direction. In some embodiments, the ALK-PAQR4 fusion polypeptide of the disclosure comprises exons 1-2, and exon 3 or a portion thereof, of ALK fused to exon 1 or a portion thereof, and exons 2-3, of PAQR4, e.g., in the amino-terminus to carboxyl-terminus direction. In some embodiments, the ALK-APH1A fusion polypeptide of the disclosure comprises exons 1-3, and exon 4 or a portion thereof, of ALK fused to exon 4 or a portion thereof, and exons 5-6, of APH1A, e.g., in the amino-terminus to carboxyl-terminus direction.

In some embodiments, an ALK fusion polypeptide of the disclosure comprises the corresponding amino acid sequence as listed in Table 8, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

TABLE 8
ALK fusion polypeptide amino acid sequences.

| ALK Fusion | | SEQ ID |
|---|---|---|
| Polypeptide | ALK Fusion Polypeptide Amino Acid Sequence | NO |
| ALK- | MMSFVQKGSWLLLALLHPTIILAQQEAVEGGCSHLGQSYADRDVWKPEPCQICV | 44 |
| COL3A1 | CDSGSVLCDDIICDDQELDCPNPEIPFGECCAVCPQPPTAPTRPPNGQGPQGPK | |
| | GDPGPPGIPGRNGDPGIPGQPGSPGSPGPPGICESCPTGPQNYSPQYDSYDVKS | |
| | GVAVGGLAGYPGPAGPPGPPGPPGTSGHPGSPGSPGYQGPPGEPGQAGPSGPPG | |
| | PPGAIGPSGPAGKDGESGRPGRPGERGLPGPPGIKGPAGIPGFPGMKGHRGFDG | |
| | RNGEKGETGAPGLKGENGLPGENGAPGPMGPRGAPGERGRPGLPGAAGARGNDG | |
| | ARGSDGQPGPPGPPGTAGFPGSPGAKGEVGPAGSPGSNGAPGQRGEPGPQGHAG | |
| | AQGPPGPPGINGSPGGKGEMGPAGIPGAPGLMGARGPPGPAGANGAPGLRGGAG | |
| | EPGKNGAKGEPGPRGERGEAGIPGVPGAKGEDGKDGSPGEPGANGLPGAAGERG | |
| | APGFRGPAGPNGIPGEKGPAGERGAPGPAGPRGAAGEPGRDGVPGGPGMRGMPG | |
| | SPGGPGSDGKPGPPGSQGESGRPGPPGPSGPRGQPGVMGFPGPKGNDGAPGKNG | |
| | ERGGPGGPGPQGPPGKNGETGPQGPPGPTGPGGDKGDTGPPGPQGLQGLPGTGG | |
| | PPGENGKPGEPGPKGDAGAPGAPGGKGDAGAPGERGPPGLAGAPGLRGGAGPPG | |
| | PEGGKGAAGPPGPPGAAGTPGLQGMPGERGGLGSPGPKGDKGEPGGPGADGVPG | |
| | KDGPRGPTGPIGPPGPAGQPGDKGEGGAPGLPGIAGPRGSPGERGETGPPGPAG | |
| | FPGAPGQNGEPGGKGERGAPGEKGEGGPPGVAGPPGGSGPAGPPGPQGVKGERG | |
| | SPGGPGAAGFPGARGLPGPPGSNGNPGPPGPSGSPGKDGPPGPAGNTGAPGSPG | |
| | VSGPKGDAGQPGEKGSPGAQGPPGAPGPLGIAGITGARGLAGPPGMPGPRGSPG | |
| | PQGVKGESGKPGANGLSGERGPPGPQGLPGLAGTAGEPGRDGNPGSDGLPGRDG | |
| | SPGGKGDRGENGSPGAPGAPGHPGPPGPVGPAGKSGDRGESGPAGPAGAPGPAG | |
| | SRGAPGPQGPRGDKGETGERGAAGIKGHRGFPGNPGAPGSPGPAGQQGAIGSPG | |
| | PAGPRGPVGPSGPPGKDGTSGHPGPIGPPGPRGNRGERGSEGSPGHPGQPGPPG | |
| | PPGAPGPCCGGVGAAAIAGIGGEKAGGFAPYYGDEPMDFKINTDEIMTSLKSVN | |
| | GQIESLISPDGSRKNPARNCRDLKFCHPELKSVSPTPEPHLPLSLILSVVTSAL | |
| | VAALVLAFSGIMIVYRRKHQELQAMQMELQSPEYKLSKLRTSTIMTDYNPNYCF | |
| | AGKTSSISDLKEVPRKNITLIRGLGHGAFGEVYEGQVSGMPNDPSPLQVAVKTL | |
| | PEVCSEQDELDELMEALIISKFNHQNIVRCIGVSLQSLPRFILLELMAGGDLKS | |
| | FLRETRPRPSQPSSLAMLDLLHVARDIACGCQYLEENHFIHRDIAARNCLLTCP | |
| | GPGRVAKIGDFGMARDIYRASYYRKGGCAMLPVKWMPPEAFMEGIFTSKIDTWS | |
| | FGVLLWEIFSLGYMPYPSKSNQEVLEFVTSGGRMDPPKNCPGPVYRIMTQCWQH | |
| | QPEDRPNFAIILERIEYCTQDPDVINTALPIEYGPLVEEEEKVPVRPKDPEGVP | |
| | PLLVSQQAKREEERSPAAPPPLPTTSSGKAAKKPTAAEISVRVPRGPAVEGGHV | |
| | NMAFSQSNPPSELHKVHGSRNKPTSLWNPTYGSWFTEKPTKKNNPIAKKEPHDR | |
| | GNLGLEGSCTVPPNVATGRLPGASLLLEPSSLTANMKEVPLFRLRHFPCGNVNY | |
| | GYQQQGLPLEAATAPGAGHYEDTILKSKNSMNQPGP | |
| ALK- | MSGEVRLRQLEQFILDGPAQTNGQCFSVETLLDILICLYDECNNSPLRREKNIL | 45 |
| CDC42BPA | EYLEWAKPFTSKVKQMRLHREDFEILKVIGRGAFGEVAVVKLKNADKVFAMKIL | |
| | NKWEMLKRAETACFREERDVLVNGDNKWITTLHYAFQDDNNLYLVMDYYVGGDL | |
| | LTLLSKFEDRLPEDMARFYLAEMVIAIDSVHQLHYVHRDIKPDNILMDMNGHIR | |
| | LADFGSCLKLMEDGTVQSSVAVGTPDYISPEILQAMEDGKGRYGPECDWWSLGV | |
| | CMYEMLYGETPFYAESLVETYGKIMNHKERFQFPAQVTDVSENAKDLIRRLICS | |
| | REHRLGQNGIEDFKKHPFFSGIDWDNIRNCEAPYIPEVSSPTDTSNFDVDDDCL | |
| | KNSETMPPPTHTAFSGHHLPFVGFTYTSSCVLSDRSCLRVTAGPTSLDLDVNVQ | |
| | RTLDNNLATEAYERRIKRLEQEKLELSRKLQESTQTVQALQYSTVDGPLTASKD | |
| | LEIKNLKEEIEKLRKQVTESSHLEQQLEEANAVRQELDDAFRQIKAYEKQIKTL | |
| | QQEREDLNKELVQASERLKNQSKELKDAHCQRKLAMQEFMEINERLTELHTQKQ | |
| | KLARHVRDKEEEVDLVMQKVESLRQELRRTERAKKELEVHTEALAAEASKDRKL | |
| | REQSEHYSKQLENELEGLKQKQISYSPGVCSIEHQQEITKLKTDLEKKSIFYEE | |
| | ELSKREGIHANEIKNLKKELHDSEGQQLALNKEIMILKDKLEKTRRESQSEREE | |
| | FESEFKQQYEREKVLLTEENKKLTSELDKLTTLYENLSIHNQQLEEEVKDLADK | |
| | KESVAHWEAQITEIIQWVSDEKDARGYLQALASKMTEELEALRNSSLGTRATDM | |
| | PWKMRRFAKLDMSARLELQSALDAEIRAKQAIQEELNKVKASNIITECKLKDSE | |
| | KKNLELLSEIEQLIKDTEELRSEKVYRRKHQELQAMQMELQSPEYKLSKLRTST | |
| | IMTDYNPNYCFAGKTSSISDLKEVPRKNITLIRGLGHGAFGEVYEGQVSGMPND | |
| | PSPLQVAVKTLPEVCSEQDELDELMEALIISKENHQNIVRCIGVSLQSLPRFIL | |
| | LELMAGGDLKSFLRETRPRPSQPSSLAMLDLLHVARDIACGCQYLEENHFIHRD | |
| | IAARNCLLTCPGPGRVAKIGDFGMARDIYRASYYRKGGCAMLPVKWMPPEAFME | |
| | GIFTSKTDTWSFGVLLWEIFSLGYMPYPSKSNQEVLEFVTSGGRMDPPKNCPGP | |
| | VYRIMTQCWQHQPEDRPNFAIILERIEYCTQDPDVINTALPIEYGPLVEEEEKV | |
| | PVRPKDPEGVPPLLVSQQAKREEERSPAAPPPLPTTSSGKAAKKPTAAEISVRV | |
| | PRGPAVEGGHVNMAFSQSNPPSELHKVHGSRNKPTSLWNPTYGSWFTEKPTKKN | |
| | NPIAKKEPHDRGNLGLEGSCTVPPNVATGRLPGASLLLEPSSLTANMKEVPLER | |
| | LRHFPCGNVNYGYQQQGLPLEAATAPGAGHYEDTILKSKNSMNQPGP | |
| ALK- | MELQAARACFALLWGCALAAAAAAQGKEVVLLDFAAAGGELGWLTHPYGKGWDL | 46 |
| EPHA2 | MQNIMNDMPIYMYSVCNVMSGDQDNWLRTNWVYRGEAERIFIELKFTVRDCNSF | |
| | PGGASSCKETFNLYYAESDLDYGTNFQKRLFTKIDTIAPDEITVSSDFEARHVK | |
| | LNVEERSVGPLTRKGFYLAFQDIGACVALLSVRVYYKKCPELLQGLAHFPETIA | |
| | GSDAPSLATVAGTCVDHAVVPPGGEEPRMHCAVDGEWLVPIGQCLCQAGYEKVE | |
| | DACQVSPTPEPHLPLSLILSVVTSALVAALVLAFSGIMIVYRRKHQELQAMQME | |
| | LQSPEYKLSKLRTSTIMTDYNPNYCFAGKTSSISDLKEVPRKNITLIRGLGHGA | |
| | FGEVYEGQVSGMPNDPSPLQVAVKTLPEVCSEQDELDELMEALIISKENHQNIV | |
| | RCIGVSLQSLPRFILLELMAGGDLKSFLRETRPRPSQPSSLAMLDLLHVARDIA | |
| | CGCQYLEENHFIHRDIAARNCLLTCPGPGRVAKIGDFGMARDIYRASYYRKGGC | |
| | AMLPVKWMPPEAFMEGIFTSKTDTWSFGVLLWEIFSLGYMPYPSKSNQEVLEFV | |
| | TSGGRMDPPKNCPGPVYRIMTQCWQHQPEDRPNFAIILERIEYCTQDPDVINTA | |
| | LPIEYGPLVEEEEKVPVRPKDPEGVPPLLVSQQAKREEERSPAAPPPLPTTSSG | |
| | KAAKKPTAAEISVRVPRGPAVEGGHVNMAFSQSNPPSELHKVHGSRNKPTSLWN | |
| | PTYGSWFTEKPTKKNNPIAKKEPHDRGNLGLEGSCTVPPNVATGRLPGASLLLE | |
| | PSSLTANMKEVPLFRLRHFPCGNVNYGYQQQGLPLEAATAPGAGHYEDTILKSK | |
| | NSMNQPGP | |
| ALK- | MAVAELYTQYNRVWIPDPEEVWKSAEIAKDYRVGDKVLRLLLEDGTELDYSVNP | 47 |
| MYO5C | ESLPPLRNPDILVGENDLTALSYLHEPAVLHNLRIRFAESKLIYTYSGIILVAM | |
| | NPYKQLPIYGDAIIHAYSGQNMGDMDPHIFAVAEEAYKQMARNNRNQSIIVSGE | |
| | SGAGKTVSARYAMRYFATVSKSGSNAHVEDKVLASNPITEAVGNAKTTRNDNSS | |
| | RFGKYTEISFDEQNQIIGANMSTYLLEKSRVVFQSENERNYHIFYQLCASAQQS | |
| | EFKHLKLGSAEEFNYTRMGGNTVIEGVNDRAEMVETQKTFTLLGFKEDFQMDVF | |
| | KILAAILHLGNVQITAVGNERSSVSEDDSHLKVFCELLGLESGRVAQWLCNRKI | |
| | VTSSETVVKPMTRPQAVNARDALAKKIYAHLFDFIVERINQALQFSGKQHTFIG | |
| | VLDIYGFETFDVNSFEQFCINYANEKLQQQFNMHVFKLEQEEYMKEDIPWTLID | |
| | FYDNQPVIDLIEAKMGILELLDEECLLPHGTDENWLQKLYNNFVNRNPLFEKPR | |
| | MSNTSFVIQHFADKVEYKCEGFLEKNRDTVYDMLVEILRASKFHLCANFFQENP | |
| | TPPSPFGSMITVKSAKQVIKPNSKHFRTTVGSKFRSSLYLLMETLNATTPHYVR | |
| | CIKPNDEKLPFEFDSKRIVQQLRACGVLETIRISAQSYPSRWTYIEFYSRYGIL | |
| | MTKQELSFSDKKEVCKVVLHRLIQDSNQYQFGKTKIFFRAGQVAYLEKLRLDKL | |
| | RQSCVMVQKHMRGWLQRKKFLRERRAALIIQQYFRGQQTVRKAITAVALKEAWA | |
| | AIIIQKHCRGYLVRSLYQLIRMATITMQAYSRGFLARRRYRKMLEEHKAVILQK | |
| | YARAWLARRRFQSIRRFVLNIQLTYRVQRLQKKLEDQNKENHGLVEKLTSLAAL | |
| | RAGDVEKIQKLEAELEKAATHRRNYEEKGKRYRDAVEEKLAKLQKHNSELETQK | |
| | EQIQLKLQEKTEELKEKMDNLTKQLFDDVQKEERQRMLLEKSFELKTQDYEKQI | |
| | QSLKEEIKALKDEKMQLQHLVEGEHVTSDGLKAEVARLSKQVKTISEFEKEIEL | |
| | LQAQKIDVEKHVQSQKREMREKMSEITKQLLESYDIEDVRSRLSVEDLEHLNED | |
| | GELWFAYEGLKKATRVLESHFQSQKDCYEKEIEALNFKVVHLSQEINHLQKLER | |
| | EENDINESIRHEVTRLISENMMIPDFKQQISELEKQKQDLEIRLNEQAEKMKVY | |
| | RRKHQELQAMQMELQSPEYKLSKLRISTIMTDYNPNYCFAGKTSSISDLKEVPR | |
| | KNITLIRGLGHGAFGEVYEGQVSGMPNDPSPLQVAVKTLPEVCSEQDELDELME | |
| | ALIISKENHQNIVRCIGVSLQSLPRFILLELMAGGDLKSFLRETRPRPSQPSSL | |
| | AMLDLLHVARDIACGCQYLEENHFIHRDIAARNCLLTCPGPGRVAKIGDFGMAR | |
| | DIYRASYYRKGGCAMLPVKWMPPEAFMEGIFTSKIDTWSFGVLLWEIFSLGYMP | |
| | YPSKSNQEVLEFVTSGGRMDPPKNCPGPVYRIMTQCWQHQPEDRPNFAIILERI | |
| | EYCTQDPDVINTALPIEYGPLVEEEEKVPVRPKDPEGVPPLLVSQQAKREEERS | |
| | PAAPPPLPTTSSGKAAKKPTAAEISVRVPRGPAVEGGHVNMAFSQSNPPSELHK | |
| | VHGSRNKPTSLWNPTYGSWFTEKPTKKNNPIAKKEPHDRGNLGLEGSCTVPPNV | |
| | ATGRLPGASLLLEPSSLTANMKEVPLFRLRHFPCGNVNYGYQQQGLPLEAATAP | |
| | GAGHYEDTILKSKNSMNQPGP | |
| ALK- | MEVAVEKAVAAAAAASAAASGGPSAAPSGENEAESRQGPDSERGGEAARLNLLD | 48 |
| TRIM24 | TCAVCHQNIQSRAPKLLPCLHSFCQRCLPAPQRYLMLPAPMLGSAETPPPVPAP | |
| | GSPVSGSSPFATQVGVIRCPVCSQECAERHIIDNFFVKDTTEVPSSTVEKSNQV | |
| | CTSCEDNAEANGFCVECVEWLCKTCIRAHQRVKFTKDHTVRQKEEVSPEAVGVT | |
| | SQRPVFCPFHKKEQLKLYCETCDKLTCRDCQLLEHKEHRYQFIEEAFQNQKVII | |
| | DTLITKLMEKTKYIKFTGNQIQNRIIEVNQNQKQVEQDIKVAIFTLMVEINKKG | |
| | KALLHQLESLAKDHRMKLMQQQQEVAGLSKQLEHVMHFSKWAVSSGSSTALLYS | |
| | KRLITYRLRHLLRARCDASPVTNNTIQFHCDPSFWAQNIINLGSLVIEDKESQP | |
| | QMPKQNPVVEQNSQPPSGLSSNQLSKFPTQISLAQLRLQHMQQQQPPPRLINFQ | |
| | NHSPKPNGPVLPPHPQQLRYPPNQNIPRQAIKPNPLQMAFLAQQAIKQWQISSG | |
| | QGTPSTTNSTSSTPSSPTITSAAGYDGKAFGSPMIDLSSPVGGSYNLPSLPDID | |
| | CSSTIMLDNIVRKDTNIDHGQPRPPSNRTVQSPNSSVPSPGLAVYRRKHQELQA | |
| | MQMELQSPEYKLSKLRISTIMTDYNPNYCFAGKTSSISDLKEVPRKNITLIRGL | |
| | GHGAFGEVYEGQVSGMPNDPSPLQVAVKTLPEVCSEQDELDELMEALIISKENH | |
| | QNIVRCIGVSLQSLPRFILLELMAGGDLKSFLRETRPRPSQPSSLAMLDLLHVA | |
| | RDIACGCQYLEENHFIHRDIAARNCLLTCPGPGRVAKIGDFGMARDIYRASYYR | |
| | KGGCAMLPVKWMPPEAFMEGIFTSKIDTWSFGVLLWEIFSLGYMPYPSKSNQEV | |
| | LEFVTSGGRMDPPKNCPGPVYRIMTQCWQHQPEDRPNFAIILERIEYCTQDPDV | |
| | INTALPIEYGPLVEEEEKVPVRPKDPEGVPPLLVSQQAKREEERSPAAPPPLPT | |
| | TSSGKAAKKPTAAEISVRVPRGPAVEGGHVNMAFSQSNPPSELHKVHGSRNKPT | |
| | SLWNPTYGSWFTEKPTKKNNPIAKKEPHDRGNLGLEGSCTVPPNVATGRLPGAS | |
| | LLLEPSSLTANMKEVPLFRLRHFPCGNVNYGYQQQGLPLEAATAPGAGHYEDTI | |
| | LKSKNSMNQPGP | |
| ALK- | MQAAALPEEIRWLLEDAEEFLAEGLRNENLSAVARDHRDHILRGFQQIKARYYW | 49 |
| SKAP1 | DFQPQVYRRKHQELQAMQMELQSPEYKLSKLRTSTIMTDYNPNYCFAGKTSSIS | |
| | DLKEVPRKNITLIRGLGHGAFGEVYEGQVSGMPNDPSPLQVAVKTLPEVCSEQD | |
| | ELDFLMEALIISKFNHQNIVRCIGVSLQSLPRFILLELMAGGDLKSFLRETRPR | |
| | PSQPSSLAMLDLLHVARDIACGCQYLEENHFIHRDIAARNCLLTCPGPGRVAKI | |
| | GDFGMARDIYRASYYRKGGCAMLPVKWMPPEAFMEGIFTSKTDTWSFGVLLWEI | |
| | FSLGYMPYPSKSNQEVLEFVTSGGRMDPPKNCPGPVYRIMTQCWQHQPEDRPNF | |
| | AIILERIEYCTQDPDVINTALPIEYGPLVEEEEKVPVRPKDPEGVPPLLVSQQA | |
| | KREEERSPAAPPPLPTTSSGKAAKKPTAAEISVRVPRGPAVEGGHVNMAFSQSN | |
| | PPSELHKVHGSRNKPTSLWNPTYGSWFTEKPTKKNNPIAKKEPHDRGNLGLEGS | |
| | CTVPPNVATGRLPGASLLLEPSSLTANMKEVPLFRLRHFPCGNVNYGYQQQGLP | |
| | LEAATAPGAGHYEDTILKSKNSMNQPGP | |
| ALK- | MFTLSQTSRAWFIDRARQAREERLVQKERERAAVVIQAHVRSFLCRSRLQRDIR | 50 |
| UBE3B | REIDDFFKADDPESTKRSALCIFKIARKLLFLFRIKEDNERFEKLCRSILSSMD | |
| | AENEPKVWYVSLACSKDLTLLWIQQIKNILWYCCDFLKQLKPEILQDSRLITLY | |
| | LTMLVTFTDTSTWKILRGKGESLRPAMNHICANIMGHLNQHGFYSVLQILLTRG | |
| | LARPRPCLSKGTLTAAFSLALRPVIAAQFSDNLIRPFLIHIMSVPALVTHLSTV | |
| | TPERLTVLESHDMLRKFIIFLRDQDRCRDVCESLEGCHTLCLMVYRRKHQELQA | |
| | MQMELQSPEYKLSKLRTSTIMTDYNPNYCFAGKTSSISDLKEVPRKNITLIRGL | |
| | GHGAFGEVYEGQVSGMPNDPSPLQVAVKTLPEVCSEQDELDELMEALIISKFNH | |
| | QNIVRCIGVSLQSLPRFILLELMAGGDLKSFLRETRPRPSQPSSLAMLDLLHVA | |
| | RDIACGCQYLEENHFIHRDIAARNCLLTCPGPGRVAKIGDFGMARDIYRASYYR | |
| | KGGCAMLPVKWMPPEAFMEGIFTSKTDTWSFGVLLWEIFSLGYMPYPSKSNQEV | |
| | LEFVTSGGRMDPPKNCPGPVYRIMTQCWQHQPEDRPNFAIILERIEYCTQDPDV | |
| | INTALPIEYGPLVEEEEKVPVRPKDPEGVPPLLVSQQAKREEERSPAAPPPLPT | |
| | TSSGKAAKKPTAAEISVRVPRGPAVEGGHVNMAFSQSNPPSELHKVHGSRNKPT | |
| | SLWNPTYGSWFTEKPTKKNNPIAKKEPHDRGNLGLEGSCTVPPNVATGRLPGAS | |
| | LLLEPSSLTANMKEVPLFRLRHFPCGNVNYGYQQQGLPLEAATAPGAGHYEDTI | |
| | LKSKNSMNQPGP | |
| ALK-TNS3 | MEEGHGLDLTYITERIIAVSFPAGCSEESYLHNLQEVTRMLKSKHGDNYLVLNL | 51 |
| | SEKRYDLTKLNPKIMDVGWPELHAPPLDKMCTICKAQESWLNSNLQHVVVIHCR | |
| | GGKGRIGVVISSYMHFTNVSASADQALDRFAMKKFYDDKVSALMQPSQKRYVQF | |
| | LSGLLSGSVKMNASPLFLHFVILHGTPNFDTGGVCRPFLKLYQAMQPVYTSGIY | |
| | NVGPENPSRICIVIEPAQLLKGDVMVKCYHKKYRSATRDVIFRLQFHTGAVQGY | |
| | GLVFGKEDLDNASKDDRFPDYGKVELVFSATPEKIQGSEHLYNDHGVIVDYNTT | |
| | DPLIRWDSYENLSADGEVLHTQGPVDGSLYAKVRKKSSSDPGIPGGPQAIPATN | |
| | SPDHSDHTLSVSSDSGHSTASARTDKTEERLAPGTRRGLSAQEKAELDQLLSGF | |
| | GLEDPGSSLKEMTDARSKYSGTRHVVPAQVHVNGDAALKDRETDILDDEMPHHD | |
| | LHSVDSLGTLSSSEGPQSAHLGPFTCHKSSQNSLLSDGFGSNVGEDPQGTLVPD | |
| | LGLGMDGPYERERTFGSREPKQPQPLLRKPSVSAQMQAYGQSSYSTQTWVRQQQ | |
| | MVVAHQYSFAPDGEARLVSRCPADNPGLVQAQPRVPLTPTRGTSSRVAVQRGVG | |
| | SGPHPPDTQQPSPSKAFKPRFPGDQVVNGAGPELSTGPSPGSPTLDIDQSIEQL | |
| | NRLILELDPTFEPIPTHMNALGSQANGSVSPDSVGGGLRASSRLPDTGEGPSRA | |
| | TGRQGSSAEQPLGGRLRKLSLGQYDNDAGGQLPFSKCAWGKAGVDYAPNLPPFP | |
| | SPADVKETMTPGYPQDLDIIDGRILSSKESMCSTPAFPVSPETPYVKTALRHPP | |
| | FSPPEPPLSSPASQHKGGREPRSCPETLTHAVGMSESPIGPKSTMLRADASSTP | |
| | SFQQAFASSCTISSNGPGQRRESSSSAERQWVESSPKPMVSLLGSGRPTGSPLS | |
| | AEFSGTRKDSPVLSCFPPSELQAPFHSHELSLAEPPDSLAPPSSQAFLGFGTAP | |
| | VGSGLPPEEDLGALLANSHGASPTPSIPLTATGAADNGFLSHNFLTVAPGHSSH | |
| | HSPGLQGQGVTLPGQPPLPEKKRASEGDRSLGSVSPSSSGFSSPHSGSTISIPF | |
| | PNVLPDFSKASEAASPLPDSPGDKLVIVKFVQDTSKFWYKADISREQAIAMLKD | |
| | KEPGSFIVRDSHSFRGAYGLAMKVATPPPSVLQLNKKAGDLANELVRHFLIECT | |
| | PKGVRLKGCSNEPYFVYRRKHQELQAMQMELQSPEYKLSKLRTSTIMTDYNPNY | |
| | CFAGKTSSISDLKEVPRKNITLIRGLGHGAFGEVYEGQVSGMPNDPSPLQVAVK | |
| | TLPEVCSEQDELDFLMEALIISKENHQNIVRCIGVSLQSLPRFILLELMAGGDL | |
| | KSFLRETRPRPSQPSSLAMLDLLHVARDIACGCQYLEENHFIHRDIAARNCLLT | |
| | CPGPGRVAKIGDFGMARDIYRASYYRKGGCAMLPVKWMPPEAFMEGIFTSKTDT | |
| | WSFGVLLWEIFSLGYMPYPSKSNQEVLEFVTSGGRMDPPKNCPGPVYRIMTQCW | |
| | QHQPEDRPNFAIILERIEYCTQDPDVINTALPIEYGPLVEEEEKVPVRPKDPEG | |
| | VPPLLVSQQAKREEERSPAAPPPLPTTSSGKAAKKPTAAEISVRVPRGPAVEGG | |
| | HVNMAFSQSNPPSELHKVHGSRNKPTSLWNPTYGSWFTEKPTKKNNPIAKKEPH | |
| | DRGNLGLEGSCTVPPNVATGRLPGASLLLEPSSLTANMKEVPLFRLRHFPCGNV | |
| | NYGYQQQGLPLEAATAPGAGHYEDTILKSKNSMNQPGP | |
| ALK- | MEEKEDKHQQHKIEDAAITYVSENEEIKHEEKPGKSIHHSKSHVGRGRIYYAKF | 52 |
| C2orf73 | INTNARTYNEPFPYIDPKKGPEIQGDWWSHGKALEPVFLPPYDSKSTQRSDFQK | |
| | PSCPLVLPVKHSKMQKPSCGIVYRRKHQELQAMQMELQSPEYKLSKLRTSTIMT | |
| | DYNPNYCFAGKTSSISDLKEVPRKNITLIRGLGHGAFGEVYEGQVSGMPNDPSP | |
| | LQVAVKTLPEVCSEQDELDELMEALIISKENHQNIVRCIGVSLQSLPRFILLEL | |
| | MAGGDLKSFLRETRPRPSQPSSLAMLDLLHVARDIACGCQYLEENHFIHRDIAA | |
| | RNCLLTCPGPGRVAKIGDFGMARDIYRASYYRKGGCAMLPVKWMPPEAFMEGIF | |
| | TSKTDTWSFGVLLWEIFSLGYMPYPSKSNQEVLEFVTSGGRMDPPKNCPGPVYR | |
| | IMTQCWQHQPEDRPNFAIILERIEYCTQDPDVINTALPIEYGPLVEEEEKVPVR | |
| | PKDPEGVPPLLVSQQAKREEERSPAAPPPLPTTSSGKAAKKPTAAEISVRVPRG | |
| | PAVEGGHVNMAFSQSNPPSELHKVHGSRNKPTSLWNPTYGSWFTEKPTKKNNPI | |
| | AKKEPHDRGNLGLEGSCTVPPNVATGRLPGASLLLEPSSLTANMKEVPLFRLRH | |
| | FPCGNVNYGYQQQGLPLEAATAPGAGHYEDTILKSKNSMNQPGP | |
| ALK-AZI2 | MDALVEDDICILNHEKAHKRDTVTPVSIYSGDESVASHFALVTAYEDIKKRLKD | 53 |
| | SEKENSLLKKRIRFLEEKLIARFEEETSSVGREQVNKAYHAYREVCIDRDNLKS | |
| | KLDKMNKDNSESLKVLNEQLQSKEVELLQLRTEVETQQVMRNLNPPSSNWEVEK | |
| | LSCDLKIHGLEQELELMRKECSDLKIELQKAKQTDPYQEDNLKSRDLQKLSISS | |
| | DNMQHAYWELKREMSNLHLVTQVQAELLRKLKTSTAIKKVYRRKHQELQAMQME | |
| | LQSPEYKLSKLRTSTIMTDYNPNYCFAGKTSSISDLKEVPRKNITLIRGLGHGA | |
| | FGEVYEGQVSGMPNDPSPLQVAVKTLPEVCSEQDELDELMEALIISKENHQNIV | |
| | RCIGVSLQSLPRFILLELMAGGDLKSFLRETRPRPSQPSSLAMLDLLHVARDIA | |
| | CGCQYLEENHFIHRDIAARNCLLTCPGPGRVAKIGDFGMARDIYRASYYRKGGC | |
| | AMLPVKWMPPEAFMEGIFTSKTDTWSFGVLLWEIFSLGYMPYPSKSNQEVLEFV | |
| | TSGGRMDPPKNCPGPVYRIMTQCWQHQPEDRPNFAIILERIEYCTQDPDVINTA | |
| | LPIEYGPLVEEEEKVPVRPKDPEGVPPLLVSQQAKREEERSPAAPPPLPTTSSG | |
| | KAAKKPTAAEISVRVPRGPAVEGGHVNMAFSQSNPPSELHKVHGSRNKPTSLWN | |
| | PTYGSWFTEKPTKKNNPIAKKEPHDRGNLGLEGSCTVPPNVATGRLPGASLLLE | |
| | PSSLTANMKEVPLFRLRHFPCGNVNYGYQQQGLPLEAATAPGAGHYEDTILKSK | |
| | NSMNQPGP | |
| ALK- | MRLHLLLLLALCGAGTTAAELSYSLRGNWSICNGNGSLELPGAVPGCVHSALFQ | 54 |
| MANBA | QGLIQDSYYRENDLNYRWVSLDNWTYSKEFKIPFEISKWQKVNLILEGVDTVSK | |
| | ILFNEVTIGETDNMFNRYSFDITNVVRDVNSIELRFQSAVLYAAQQSKAHTRYQ | |
| | VPPDCPPLVQKGECHVNFVRKEQCSFSWDWGPSFPTQGIWKDVRIEAYNICHLN | |
| | YFTFSPIYDKSAQEWNLEIESTFDVVSSKPVGGQVIVAIPKLQTQQTYSIELQP | |
| | GKRIVELFVNISKNITVETWWPHGHGNQTGYNMTVLFELDGGLNIEKSAKVYFR | |
| | TVELIEEPIKGSPGLSFYFKINGFPIFLKGSNWIPADSFQDRVTSELLRLLLQS | |
| | VVDANMNTLRVWGGGIYEQDEFYELCDELGIMVWQDFMFACALYPTDQGELDSV | |
| | TAEVAYQIKRLKSHPSIIIWSGNNENEEALMMNWYHISFTDRPIYIKDYVTLYV | |
| | KNIRELVLAGDKSRPFITSSPTNGAETVAEAWVSQNPNSNYFGDVHFYDYISDC | |
| | WNWKVFPKARFASEYGYQSWPSFSTLEKVSSTEDWSFNSKFSLHRQHHEGGNKQ | |
| | MLYQAGLHFKLPQSTDPLRTFKDTIYLTQVMQAQCVKTETEFYRRSRSEIVDQQ | |
| | GHTMGALYWQLNDIWQAPSWASLVYRRKHQELQAMQMELQSPEYKLSKLRISTI | |
| | MTDYNPNYCFAGKTSSISDLKEVPRKNITLIRGLGHGAFGEVYEGQVSGMPNDP | |
| | SPLQVAVKTLPEVCSEQDELDFLMEALIISKENHQNIVRCIGVSLQSLPRFILL | |
| | ELMAGGDLKSFLRETRPRPSQPSSLAMLDLLHVARDIACGCQYLEENHFIHRDI | |
| | AARNCLLTCPGPGRVAKIGDFGMARDIYRASYYRKGGCAMLPVKWMPPEAFMEG | |
| | IFTSKTDTWSFGVLLWEIFSLGYMPYPSKSNQEVLEFVTSGGRMDPPKNCPGPV | |
| | YRIMTQCWQHQPEDRPNFAIILERIEYCTQDPDVINTALPIEYGPLVEEEEKVP | |
| | VRPKDPEGVPPLLVSQQAKREEERSPAAPPPLPTTSSGKAAKKPTAAEISVRVP | |
| | RGPAVEGGHVNMAFSQSNPPSELHKVHGSRNKPTSLWNPTYGSWFTEKPTKKNN | |
| | PIAKKEPHDRGNLGLEGSCTVPPNVATGRLPGASLLLEPSSLTANMKEVPLFRL | |
| | RHFPCGNVNYGYQQQGLPLEAATAPGAGHYEDTILKSKNSMNQPGP | |
| ALK- | MDSLPRLTSVLTLLFSGLWHLGLTATNYNCDDPLASLLSPMAFSSSSDLIGTHS | 55 |
| CNTNAP5 | PAQLNWRVVYRRKHQELQAMQMELQSPEYKLSKLRTSTIMTDYNPNYCFAGKTS | |
| | SISDLKEVPRKNITLIRGLGHGAFGEVYEGQVSGMPNDPSPLQVAVKTLPEVCS | |
| | EQDELDELMEALIISKENHQNIVRCIGVSLQSLPRFILLELMAGGDLKSFLRET | |
| | RPRPSQPSSLAMLDLLHVARDIACGCQYLEENHFIHRDIAARNCLLTCPGPGRV | |
| | AKIGDFGMARDIYRASYYRKGGCAMLPVKWMPPEAFMEGIFTSKTDTWSFGVLL | |
| | WEIFSLGYMPYPSKSNQEVLEFVTSGGRMDPPKNCPGPVYRIMTQCWQHQPEDR | |
| | PNFAIILERIEYCTQDPDVINTALPIEYGPLVEEEEKVPVRPKDPEGVPPLLVS | |
| | QQAKREEERSPAAPPPLPTTSSGKAAKKPTAAEISVRVPRGPAVEGGHVNMAFS | |
| | QSNPPSELHKVHGSRNKPTSLWNPTYGSWFTEKPTKKNNPIAKKEPHDRGNLGL | |
| | EGSCTVPPNVATGRLPGASLLLEPSSLTANMKEVPLFRLRHFPCGNVNYGYQQQ | |
| | GLPLEAATAPGAGHYEDTILKSKNSMNQPGP | |
| ALK- | MAARQAVGSGAQETCGLDRILEALKLLLSPGGHSSLESPTNMPSPSPDYFTWNL | 57 |
| TANGO6 | TWIMKDSFPFLSHRSRYGLECSFDFPCELEYSPPLHDLRNQSWSWRRIPSEEAS | |
| | QMDLLDGPGAERSKEMPRGSFLLLNTSADSKHTILSPWMRSSSEHCTLAVSVHR | |
| | HLQPSGRYIAQLLPHNEAAREILLMPTPGKHGWTVLQGRIGRPDNPFRVALEYI | |
| | SSGNRSLSAVDFFALKNCSEGTSPGSKMALQSSFTCWNGTVLQLGQACDFHQDC | |
| | AQGEDESQMCRKLPVGFYCNFEDGFCGWTQGTLSPHTPQWQVRTLKDARFQDHQ | |
| | DHALLLSTTDVPASESATVISATFPAPIKSSPCELRMSWLIRGVLRGNVSLVLV | |
| | ENKTGKEQGRMVWHVAAYEGLSLWQWMVLPLLDVSDREWLQMVAWWGQGSRAIV | |
| | AFDNISISLDCYLTISGEDKILQNTAPKSRNLFERNPNKELKPGENSPRQTPIF | |
| | DPTVHWLFTTCGASGPHGPTQAQCNNAYQNSNLSVEVGSEGPLKGIQIWKVPAT | |
| | DTYSISGYGAAGGKGGKNTMMRSHGVSVLGIFNLEKDDMLYILVGQQGEDACPS | |
| | TNQLIQKVCIGENNVIEEEIRVNRSVHEWAGGGGGGGGATYVFKMKDGVPVPLI | |
| | IAAGGGGRAYGAKTDTFHPERLENNSSVLGLNGNSGAAGGGGGWNDNTSLLWAG | |
| | KSLQEGATGGHSCPQAMKKWGWETRGGFGGGGGGCSSGGGGGGYIGGNAASNND | |
| | PEMDGEDGVSFISPLGILYTPALKVMEGHGEVNIKHYLNCSHCEVDECHMDPES | |
| | HKVICFCDHGTVLAEDGVSCIVSPTPEPHLPLSLILSVVTSALVAALVLAFSGI | |
| | MIVYRRKHQELQAMQMELQSPEYKLSKLRTSTIMTDYNPNYCFAGKTSSISDLK | |
| | EVPRKNITLIRGLGHGAFGEVYEGQVSGMPNDPSPLQVAVKTLPEVCSEQDELD | |
| | FLMEALIISKENHQNIVRCIGVSLQSLPRFILLELMAGGDLKSFLRETRPRPSQ | |
| | PSSLAMLDLLHVARDIACGCQYLEENHFIHRDIAARNCLLTCPGPGRVAKIGDF | |
| | GMARDIYRASYYRKGGCAMLPVKWMPPEAFMEGIFTSKTDTWSFGVLLWEIFSL | |
| | GYMPYPSKSNQEVLEFVTSGGRMDPPKNCPGPVYRIMTQCWQHQPEDRPNFAII | |
| | LERIEYCTQDPDVINTALPIEYGPLVEEEEKVPVRPKDPEGVPPLLVSQQAKRE | |
| | EERSPAAPPPLPTTSSGKAAKKPTAAEISVRVPRGPAVEGGHVNMAFSQSNPPS | |
| | ELHKVHGSRNKPTSLWNPTYGSWFTEKPTKKNNPIAKKEPHDRGNLGLEGSCTV | |
| | PPNVATGRLPGASLLLEPSSLTANMKEVPLFRLRHFPCGNVNYGYQQQGLPLEA | |
| | ATAPGAGHYEDTILKSKNSMNQPGP | |
| ALK-NFIA | MYSPLCLTQDEFHPFIEALLPHVRAFAYTWFNLQARKRKYFKKHEKRMSKEEER | 58 |
| | AVKDELLSEKPEVKQKWASRLLAKLRKDIRPEYREDFVLTVTGKKPPCCVLSNP | |
| | DQKGKMRRIDCLRQADKVWRLDLVMVILFKGIPLESTDGERLVKSPQCSNPGLC | |
| | VQPHHIGVSVKELDLYLAYFVHAAGHSSLESPTNMPSPSPDYFTWNLTWIMKDS | |
| | FPFLSHRSRYGLECSFDFPCELEYSPPLHDLRNQSWSWRRIPSEEASQMDLLDG | |
| | PGAERSKEMPRGSFLLLNTSADSKHTILSPWMRSSSEHCTLAVSVHRHLQPSGR | |
| | YIAQLLPHNEAAREILLMPTPGKHGWTVLQGRIGRPDNPFRVALEYISSGNRSL | |
| | SAVDFFALKNCSEGTSPGSKMALQSSFTCWNGTVLQLGQACDFHQDCAQGEDES | |
| | QMCRKLPVGFYCNFEDGFCGWTQGTLSPHTPQWQVRTLKDARFQDHQDHALLLS | |
| | TTDVPASESATVISATFPAPIKSSPCELRMSWLIRGVLRGNVSLVLVENKTGKE | |
| | QGRMVWHVAAYEGLSLWQWMVLPLLDVSDREWLQMVAWWGQGSRAIVAFDNISI | |
| | SLDCYLTISGEDKILQNTAPKSRNLFERNPNKELKPGENSPRQTPIFDPTVHWL | |
| | FTTCGASGPHGPTQAQCNNAYQNSNLSVEVGSEGPLKGIQIWKVPATDTYSISG | |
| | YGAAGGKGGKNTMMRSHGVSVLGIFNLEKDDMLYILVGQQGEDACPSTNQLIQK | |
| | VCIGENNVIEEEIRVNRSVHEWAGGGGGGGGATYVFKMKDGVPVPLIIAAGGGG | |
| | RAYGAKTDTFHPERLENNSSVLGLNGNSGAAGGGGGWNDNTSLLWAGKSLQEGA | |
| | TGGHSCPQAMKKWGWETRGGFGGGGGGCSSGGGGGGYIGGNAASNNDPEMDGED | |
| | GVSFISPLGILYTPALKVMEGHGEVNIKHYLNCSHCEVDECHMDPESHKVICFC | |
| | DHGTVLAEDGVSCIVSPTPEPHLPLSLILSVVTSALVAALVLAFSGIMIVYRRK | |
| | HQELQAMQMELQSPEYKLSKLRISTIMTDYNPNYCFAGKTSSISDLKEVPRKNI | |
| | TLIRGLGHGAFGEVYEGQVSGMPNDPSPLQVAVKTLPEVCSEQDELDELMEALI | |
| | ISKFNHQNIVRCIGVSLQSLPRFILLELMAGGDLKSFLRETRPRPSQPSSLAML | |
| | DLLHVARDIACGCQYLEENHFIHRDIAARNCLLTCPGPGRVAKIGDFGMARDIY | |
| | RASYYRKGGCAMLPVKWMPPEAFMEGIFTSKTDTWSFGVLLWEIFSLGYMPYPS | |
| | KSNQEVLEFVTSGGRMDPPKNCPGPVYRIMTQCWQHQPEDRPNFAIILERIEYC | |
| | TQDPDVINTALPIEYGPLVEEEEKVPVRPKDPEGVPPLLVSQQAKREEERSPAA | |
| | PPPLPTTSSGKAAKKPTAAEISVRVPRGPAVEGGHVNMAFSQSNPPSELHKVHG | |
| | SRNKPTSLWNPTYGSWFTEKPTKKNNPIAKKEPHDRGNLGLEGSCTVPPNVATG | |
| | RLPGASLLLEPSSLTANMKEVPLFRLRHFPCGNVNYGYQQQGLPLEAATAPGAG | |
| | HYEDTILKSKNSMNQPGP | |
| ALK- | MEEEGGSSGGAAGTSADGGDGGEQLLTVKHELRTDHALLLSTTDVPASESATVT | 59 |
| RPS6KA5 | SATFPAPIKSSPCELRMSWLIRGVLRGNVSLVLVENKTGKEQGRMVWHVAAYEG | |
| | LSLWQWMVLPLLDVSDREWLQMVAWWGQGSRAIVAFDNISISLDCYLTISGEDK | |
| | ILQNTAPKSRNLFERNPNKELKPGENSPRQTPIFDPTVHWLFTTCGASGPHGPT | |
| | QAQCNNAYQNSNLSVEVGSEGPLKGIQIWKVPATDTYSISGYGAAGGKGGKNTM | |
| | MRSHGVSVLGIFNLEKDDMLYILVGQQGEDACPSTNQLIQKVCIGENNVIEEEI | |
| | RVNRSVHEWAGGGGGGGGATYVFKMKDGVPVPLIIAAGGGGRAYGAKTDTFHPE | |
| | RLENNSSVLGLNGNSGAAGGGGGWNDNTSLLWAGKSLQEGATGGHSCPQAMKKW | |
| | GWETRGGFGGGGGGCSSGGGGGGYIGGNAASNNDPEMDGEDGVSFISPLGILYT | |
| | PALKVMEGHGEVNIKHYLNCSHCEVDECHMDPESHKVICFCDHGTVLAEDGVSC | |
| | IVSPTPEPHLPLSLILSVVTSALVAALVLAFSGIMIVYRRKHQELQAMQMELQS | |
| | PEYKLSKLRTSTIMTDYNPNYCFAGKTSSISDLKEVPRKNITLIRGLGHGAFGE | |
| | VYEGQVSGMPNDPSPLQVAVKTLPEVCSEQDELDELMEALIISKENHQNIVRCI | |
| | GVSLQSLPRFILLELMAGGDLKSFLRETRPRPSQPSSLAMLDLLHVARDIACGC | |
| | QYLEENHFIHRDIAARNCLLTCPGPGRVAKIGDFGMARDIYRASYYRKGGCAML | |
| | PVKWMPPEAFMEGIFTSKTDTWSFGVLLWEIFSLGYMPYPSKSNQEVLEFVTSG | |
| | GRMDPPKNCPGPVYRIMTQCWQHQPEDRPNFAIILERIEYCTQDPDVINTALPI | |
| | EYGPLVEEEEKVPVRPKDPEGVPPLLVSQQAKREEERSPAAPPPLPTTSSGKAA | |
| | KKPTAAEISVRVPRGPAVEGGHVNMAFSQSNPPSELHKVHGSRNKPTSLWNPTY | |
| | GSWFTEKPTKKNNPIAKKEPHDRGNLGLEGSCTVPPNVATGRLPGASLLLEPSS | |
| | LTANMKEVPLFRLRHFPCGNVNYGYQQQGLPLEAATAPGAGHYEDTILKSKNSM | |
| | NQPGP | |
| ALK-TG | MALVLEIFTLLASICWVSANIFEYQVDAQPLRPCELQRETAFLKQADYVPQCAE | 60 |
| | DGSFQTVQCQNDGRSCWCVGANGSEVLGSRQPGRPVACLSFCQLQKQQILLSGY | |
| | INSTDTSYLPQCQDSGDYAPVQCDVQQVQCWCVDAEGMEVYGTRQLGRPKRCPR | |
| | SCEIRNRRLLHGVGDKSPPQCSAEGEFMPVQCKFVNTTDMMIFDLVHSYNRFPD | |
| | AFVTFSSFQRRFPEVSGYCHCADSQGRELAETGLELLLDEIYDTIFAGLDLPST | |
| | FTETTLYRILQRRFLAVQSVISGRFRCPTKCEVERFTATSFGHPYVPSCRRNGD | |
| | YQAVQCQTEGPCWCVDAQGKEMHGTRQQGEPPSCAEGQSCASERQQALSRLYFG | |
| | TSGYFSQHDLFSSPEKRWASPRVARFATSCPPTIKELFVDSGLLRPMVEGQSQQ | |
| | FSVSENLLKEAIRAIFPSRGLARLALQFTTNPKRLQQNLEGGKFLVNVGQFNLS | |
| | GALGTRGTFNFSQFFQQLGLASFLNGGRQEDLAKPLSVGLDSNSSTGTPEAAKK | |
| | DGTMNKPTVGSFGFEINLQENQNALKFLASLLELPEFLLFLQHAISVPEDVARD | |
| | LGDVMETVLSSQTCEQTPERLFVPSCTTEGSYEDVQCFSGECWCVNSWGKELPG | |
| | SRVRGGQPRCPTDCEKQRARMQSLMGSQPAGSTLFVPACTSEGHELPVQCENSE | |
| | CYCVDAEGQAIPGTRSAIGKPKKCPTPCQLQSEQAFLRTVQALLSNSSMLPTLS | |
| | DTYIPQCSTDGQWRQVQCNGPPEQVFELYQRWEAQNKGQDLTPAKLLVKIMSYR | |
| | EAASGNFSLFIQSLYEAGQQDVFPVLSQYPSLQDVPLAALEGKRPQPRENILLE | |
| | PYLFWQILNGQLSQYPGSYSDFSTPLAHFDLRNCWCVDEAGQELEGMRSEPSKL | |
| | PTLSPTPEPHLPLSLILSVVTSALVAALVLAFSGIMIVYRRKHQELQAMQMELQ | |
| | SPEYKLSKLRTSTIMTDYNPNYCFAGKTSSISDLKEVPRKNITLIRGLGHGAFG | |
| | EVYEGQVSGMPNDPSPLQVAVKTLPEVCSEQDELDELMEALIISKENHQNIVRC | |
| | IGVSLQSLPRFILLELMAGGDLKSFLRETRPRPSQPSSLAMLDLLHVARDIACG | |
| | CQYLEENHFIHRDIAARNCLLTCPGPGRVAKIGDFGMARDIYRASYYRKGGCAM | |
| | LPVKWMPPEAFMEGIFTSKTDTWSFGVLLWEIFSLGYMPYPSKSNQEVLEFVTS | |
| | GGRMDPPKNCPGPVYRIMTQCWQHQPEDRPNFAIILERIEYCTQDPDVINTALP | |
| | IEYGPLVEEEEKVPVRPKDPEGVPPLLVSQQAKREEERSPAAPPPLPTTSSGKA | |
| | AKKPTAAEISVRVPRGPAVEGGHVNMAFSQSNPPSELHKVHGSRNKPTSLWNPT | |
| | YGSWFTEKPTKKNNPIAKKEPHDRGNLGLEGSCTVPPNVATGRLPGASLLLEPS | |
| | SLTANMKEVPLFRLRHFPCGNVNYGYQQQGLPLEAATAPGAGHYEDTILKSKNS | |
| | MNQPGP | |
| ALK- | MGTPASGRKRTPVKDRFSAEDEALSNIAREAEARLAAKRAARAEARDIRMRELE | 61 |
| LRRFIP2 | RQQKEYSLHSFDRKWGQIQKWLEDSERARYSHRSSHHRPYLGVEDALSIRSVGS | |
| | HRYDMFKDRSSRLSSLNHSYSHSHGMKKRSSDSHKDLLSGLYFDQRNYSSLRHS | |
| | KPTSAYYTRQSSSLYSDPLATYKSDRASPTANSGLLRSASLASLYNGGLYNPYG | |
| | PRTPSECSYYSSRISSARSSPGFTNDDTASIVSSDRASRGRRESVVSAADYFSR | |
| | SNRRGSVVSEVDDISIPDLSSLDEKSDKQYAENYTRPSSRNSASATTPLSGNSS | |
| | RRGSGDTSSLIDPDTSLSELRDIYDLKDQIQDVEGRYMQGLKELKESLSEVEEK | |
| | YKKAMVSNAQLDNEKNNLIYQVDTLKDVIEEQEEQMAEFYRENEEKSKELERQK | |
| | HMCSVLQHKMEELKEGLRQRDELIEEKQRMQQKIDTMTKEVEDLQETLLWKDKK | |
| | IGALEKQKEYIACLRNERDMLREELADLQETVKTGEKHGLVIIPDGTPNGDVSH | |
| | EPVAGAITVVSQEAAQVLESAGEGPLDVRLRKLAGEKEELLSQIRKLKLQLEEE | |
| | RQKCSRNDGTVGDLAGLQNGSDLQFIEMQMYRRKHQELQAMQMELQSPEYKLSK | |
| | LRTSTIMTDYNPNYCFAGKTSSISDLKEVPRKNITLIRGLGHGAFGEVYEGQVS | |
| | GMPNDPSPLQVAVKTLPEVCSEQDELDELMEALIISKENHQNIVRCIGVSLQSL | |
| | PRFILLELMAGGDLKSFLRETRPRPSQPSSLAMLDLLHVARDIACGCQYLEENH | |
| | FIHRDIAARNCLLTCPGPGRVAKIGDFGMARDIYRASYYRKGGCAMLPVKWMPP | |
| | EAFMEGIFTSKTDTWSFGVLLWEIFSLGYMPYPSKSNQEVLEFVTSGGRMDPPK | |
| | NCPGPVYRIMTQCWQHQPEDRPNFAIILERIEYCTQDPDVINTALPIEYGPLVE | |
| | EEEKVPVRPKDPEGVPPLLVSQQAKREEERSPAAPPPLPTTSSGKAAKKPTAAE | |
| | ISVRVPRGPAVEGGHVNMAFSQSNPPSELHKVHGSRNKPTSLWNPTYGSWFTEK | |
| | PTKKNNPIAKKEPHDRGNLGLEGSCTVPPNVATGRLPGASLLLEPSSLTANMKE | |
| | VPLFRLRHFPCGNVNYGYQQQGLPLEAATAPGAGHYEDTILKSKNSMNQPGP | |
| ALK- | MAVAELYTQYNRVWIPDPEEVWKSAEIAKDYRVGDKVLRLLLEDGTELDYSVNP | 62 |
| MYO5C | ESLPPLRNPDILVGENDLTALSYLHEPAVLHNLRIRFAESKLIYTYSGIILVAM | |
| | NPYKQLPIYGDAIIHAYSGQNMGDMDPHIFAVAEEAYKQMARNNRNQSIIVSGE | |
| | SGAGKTVSARYAMRYFATVSKSGSNAHVEDKVLASNPITEAVGNAKTTRNDNSS | |
| | RFGKYTEISFDEQNQIIGANMSTYLLEKSRVVFQSENERNYHIFYQLCASAQQS | |
| | EFKHLKLGSAEEFNYTRMGGNTVIEGVNDRAEMVETQKTFTLLGFKEDFQMDVF | |
| | KILAAILHLGNVQITAVGNERSSVSEDDSHLKVFCELLGLESGRVAQWLCNRKI | |
| | VTSSETVVKPMTRPQAVNARDALAKKIYAHLFDFIVERINQALQFSGKQHTFIG | |
| | VLDIYGFETFDVNSFEQFCINYANEKLQQQFNMHVFKLEQEEYMKEDIPWTLID | |
| | FYDNQPVIDLIEAKMGILELLDEECLLPHGTDENWLQKLYNNFVNRNPLFEKPR | |
| | MSNTSFVIQHFADKVEYKCEGFLEKNRDTVYDMLVEILRASKFHLCANFFQENP | |
| | TPPSPFGSMITVKSAKQVIKPNSKHFRTTVGSKFRSSLYLLMETLNATTPHYVR | |
| | CIKPNDEKLPFEFDSKRIVQQLRACGVLETIRISAQSYPSRWTYIEFYSRYGIL | |
| | MTKQELSFSDKKEVCKVVLHRLIQDSNQYQFGKTKIFFRAGQVAYLEKLRLDKL | |
| | RQSCVMVQKHMRGWLQRKKFLRERRAALIIQQYFRGQQTVRKAITAVALKEAWA | |
| | AIIIQKHCRGYLVRSLYQLIRMATITMQAYSRGFLARRRYRKMLEEHKAVILQK | |
| | YARAWLARRRFQSIRRFVLNIQLTYRVQRLQKKLEDQNKENHGLVEKLTSLAAL | |
| | RAGDVEKIQKLEAELEKAATHRRNYEEKGKRYRDAVEEKLAKLQKHNSELETQK | |
| | EQIQLKLQEKTEELKEKMDNLTKQLFDDVQKEERQRMLLEKSFELKTQDYEKQI | |
| | QSLKEEIKALKDEKMQLQHLVEGEHVTSDGLKAEVARLSKQVKTISEFEKEIEL | |
| | LQAQKIDVEKHVQSQKREMREKMSEITKQLLESYDIEDVRSRLSVEDLEHLNED | |
| | GELWFAYEGLKKATRVLESHFQSQKDCYEKEIEALNFKVVHLSQEINHLQKLER | |
| | EENDINESIRHEVTRLTSENMMIPDFKQQISELEKQKQDLEIRLNEQAEKMKVY | |
| | RRKHQELQAMQMELQSPEYKLSKLRTSTIMTDYNPNYCFAGKTSSISDLKEVPR | |
| | KNITLIRGLGHGAFGEVYEGQVSGMPNDPSPLQVAVKTLPEVCSEQDELDFLME | |
| | ALIISKFNHQNIVRCIGVSLQSLPRFILLELMAGGDLKSFLRETRPRPSQPSSL | |
| | AMLDLLHVARDIACGCQYLEENHFIHRDIAARNCLLTCPGPGRVAKIGDFGMAR | |
| | DIYRASYYRKGGCAMLPVKWMPPEAFMEGIFTSKTDTWSFGVLLWEIFSLGYMP | |
| | YPSKSNQEVLEFVTSGGRMDPPKNCPGPVYRIMTQCWQHQPEDRPNFAIILERI | |
| | EYCTQDPDVINTALPIEYGPLVEEEEKVPVRPKDPEGVPPLLVSQQAKREEERS | |
| | PAAPPPLPTTSSGKAAKKPTAAEISVRVPRGPAVEGGHVNMAFSQSNPPSELHK | |
| | VHGSRNKPTSLWNPTYGSWFTEKPTKKNNPIAKKEPHDRGNLGLEGSCTVPPNV | |
| | ATGRLPGASLLLEPSSLTANMKEVPLFRLRHFPCGNVNYGYQQQGLPLEAATAP | |
| | GAGHYEDTILKSKNSMNQPGP | |
| ALK- | MNYQQQLANSAAIRAEIQRFESVHPNIYSIYELLERVEEPVLQNQIREHVIAIE | 63 |
| AGAP1 | DAFVNSQEWTLSRSVPELKVGIVGNLASGKSALVHRYLTGTYVQEESPEGGRFK | |
| | KEIVVDGQSYLLLIRDEGGPPEAQFAMWVDAVIFVFSLEDEISFQTVYHYYSRM | |
| | ANYRNTSEIPLVLVGTQDAISSANPRVIDDARARKLSNDLKRCTYYETCATYGL | |
| | NVERVFQDVMEGHGEVNIKHYLNCSHCEVDECHMDPESHKVICFCDHGTVLAED | |
| | GVSCIVSPTPEPHLPLSLILSVVTSALVAALVLAFSGIMIVYRRKHQELQAMQM | |
| | ELQSPEYKLSKLRISTIMTDYNPNYCFAGKTSSISDLKEVPRKNITLIRGLGHG | |
| | AFGEVYEGQVSGMPNDPSPLQVAVKTLPEVCSEQDELDELMEALIISKENHQNI | |
| | VRCIGVSLQSLPRFILLELMAGGDLKSFLRETRPRPSQPSSLAMLDLLHVARDI | |
| | ACGCQYLEENHFIHRDIAARNCLLTCPGPGRVAKIGDFGMARDIYRASYYRKGG | |
| | CAMLPVKWMPPEAFMEGIFTSKIDTWSFGVLLWEIFSLGYMPYPSKSNQEVLEF | |
| | VTSGGRMDPPKNCPGPVYRIMTQCWQHQPEDRPNFAIILERIEYCTQDPDVINT | |
| | ALPIEYGPLVEEEEKVPVRPKDPEGVPPLLVSQQAKREEERSPAAPPPLPTTSS | |
| | GKAAKKPTAAEISVRVPRGPAVEGGHVNMAFSQSNPPSELHKVHGSRNKPTSLW | |
| | NPTYGSWFTEKPTKKNNPIAKKEPHDRGNLGLEGSCTVPPNVATGRLPGASLLL | |
| | EPSSLTANMKEVPLFRLRHFPCGNVNYGYQQQGLPLEAATAPGAGHYEDTILKS | |
| | KNSMNQPGP | |
| ALK- | MGAIGLLWLLPLLLSTAAVGSGMGTGQRAGSPAAGPPLQPREPLSYSRLQRKSL | 64 |
| MED13L | AVDFVVPSLFRVYARDLLLPPSSSELKAGRPEARGSLALDCAPLLRLLGPAPGV | |
| | SWTAGSPAPAEARTLSRVLKGGSVRKLRRAKQLVLELGEEAILEGCVGPPGEAA | |
| | VGLLQFNLSELFSWWIRQGEGRLRIRLMPEKKASEVGREGRLSAAIRASQPRLL | |
| | FQIFGTVLVSPYGLNGTLTGQAYKMSDPATRKLIEEWQYFYPMVLKKKEESKEE | |
| | DELGYDDDFPVAVEVIVGGVRMVYPSAFVLISQNDIPVPQSVASAGGHIAVGQQ | |
| | GLGSVKDPSNCGMPLTPPTSPEQAILGESGGMQSAASHLVSQDGGMITMHSPKR | |
| | SGKIPPKLHNHMVHRVWKECILNRTQSKRSQMSTPTLEEEPASNPATWDFVDPT | |
| | QRVSCSCSRHKLLKRCAVGPNRPPTVSQPGFSAGPSSSSSLPPPASSKHKTAER | |
| | QEKGDKLQKRPLIPFHHRPSVAEELCMEQDTPGQKLGLAGIDSSLEVSSSRKYD | |
| | KQMAVPSRNTSKQMNLNPMDSPHSPISPLPPTLSPQPRGQETESLDPPSVPVNP | |
| | ALYGNGLELQQLSTLDDRTVLVGQRLPLMAEVSETALYCGIRPSNPESSEKWWH | |
| | SYRLPPSDDAEFRPPELQGERCDAKMEVNSESTALQRLLAQPNKRFKIWQDKQP | |
| | QLQPLHFLDPLPLSQQPGDSLGEVNDPYTFEDGDIKYIFTANKKCKQGTEKDSL | |
| | KKNKSEDGFGTKDVTTPGHSTPVPDGKNAMSIFSSATKTDVRQDNAAGRAGSSS | |
| | LTQVTDLAPSLHDLDNIFDNSDDDELGAVSPALRSSKMPAVGTEDRPLGKDGRA | |
| | AVPYPPTVADLQRMFPTPPSLEQHPAFSPVMNYKDGISSETVTALGMMESPMVS | |
| | MVSTQLTEFKMEVEDGLGSPKPEEIKDESYVHKVPSFQPFVGSSMFAPLKMLPS | |
| | HCLLPLKIPDACLFRPSWAIPPKIEQLPMPPAATFIRDGYNNVPSVGSLADPDY | |
| | LNTPQMNTPVTLNSAAPASNSGAGVLPSPATPRFSVPTPRTPRTPRTPRGGGTA | |
| | SGQGSVKYDSTDQGSPASTPSTTRPLNSVEPATMQPIPEAHSLYVTLILSDSVM | |
| | NIFKDRNFDSCCICACNMNIKGADVGLYIPDSSNEDQYRCTCGFSAIMNRKLGY | |
| | NSGLFLEDELDIFGKNSDIGQAAERRLMMCQSTFLPQVEGTKKPQEPPISLLLL | |
| | LQNQHTQPFASLNFLDYISSNNRQTLPCVSWSYDRVQADNNDYWTECFNALEQG | |
| | RQYVDNPTGGKVDEALVRSATVHSWPHSNVLDISMLSSQDVVRMLLSLQPFLQD | |
| | AIQKKRTGRTWENIQHVQGPLTWQQFHKMAGRGTYGSEESPEPLPIPTLLVGYD | |
| | KDFLTISPFSLPFWERLLLDPYGGHRDVAYIVVCPENEALLEGAKTFFRDLSAV | |
| | YEMCRLGQHKPICKVLRDGIMRVGKTVAQKLTDELVSEWFNQPWSGEENDNHSR | |
| | LKLYAQVCRHHLAPYLATLQLDSSLLIPPKYQTPPAAAQGQATPGNAGPLAPNG | |
| | SAAPPAGSAFNPTSNSSSTNPAASSSASGSSVPPVSSSASAPGISQISTTSSSG | |
| | FSGSVGGQNPSTGGISADRTQGNIGCGGDTDPGQSSSQPSQDGQESVTERERIG | |
| | IPTEPDSADSHAHPPAVVIYMVDPFTYAAEEDSTSGNFWLLSLMRCYTEMLDNL | |
| | PEHMRNSFILQIVPCQYMLQTMKDEQVFYIQYLKSMAFSVYCQCRRPLPTQIHI | |
| | KSLTGFGPAASIEMTLKNPERPSPIQLYSPPFILAPIKDKQTELGETFGEASQK | |
| | YNVLFVGYCLSHDQRWLLASCTDLHGELLETCVVNIALPNRSRRSKVSARKIGL | |
| | QKLWEWCIGIVQMTSLPWRVVIGRLGRLGHGELKDWSILLGECSLQTISKKLKD | |
| | VCRMCGISAADSPSILSACLVAMEPQGSFVVMPDAVTMGSVFGRSTALNMQSSQ | |
| | LNTPQDASCTHILVFPTSSTIQVAPANYPNEDGESPNNDDMFVDLPFPDDMDND | |
| | IGILMTGNLHSSPNSSPVPSPGSPSGIGVGSHFQHSRSQGERLLSREAPEELKQ | |
| | QPLALGYFVSTAKAENLPQWFWSSCPQAQNQCPLFLKASLHHHISVAQTDELLP | |
| | ARNSQRVPHPLDSKTTSDVLRFVLEQYNALSWLTCNPATQDRTSCLPVHFVVLT | |
| | QLYNAIMNIL | |
| ALK- | MGAIGLLWLLPLLLSTAAVGSGMGTGQRAGSPAAGPPLQPREPLSYSRLQRKSL | 65 |
| MTBP | AVDFVVPSLFRVYARDLLLPPSSSELKAGRPEARGSLALDCAPLLRLLGPAPGV | |
| | SWTAGSPAPAEARTLSRVLKGGSVRKLRRAKQLVLELGEEAILEGCVGPPGEAA | |
| | VGLLQFNLSELFSWWIRQGEGRLRIRLMPEKKASEVGREGRLSAAIRASQPRLL | |
| | FQIFGTGHSSLESPTNMPSPSPDYFTWNLTWIMKDSFPFLSHRSRYGLECSFDF | |
| | PCELEYSPPLHDLRNQSWSWRRIPSEEASQMDLLDGPGAERSKEMPRGSFLLLN | |
| | TSADSKHTILSPWMRSSSEHCTLAVSVHRHLQPSGRYIAQLLPHNEAAREILLM | |
| | PTPGKHGWTVLQGRIGRPDNPFRVALEYISSGNRSLSAVDFFALKNCSEGTSPG | |
| | SKMALQSSFTCWNGTVLQLGQACDFHQDCAQGEDESQMCRKLPVGFYCNFEDGE | |
| | CGWTQGTLSPHTPQWQVRTLKDARFQDHQDHALLLSTTDVPASESATVTSATFP | |
| | APIKSSPCELRMSWLIRGVLRGNVSLVLVENKTGKEQGRMVWHVAAYEGLSLWQ | |
| | WMVLPLLDVSDREWLQMVAWWGQGSRAIVAFDNISISLDCYLTISGEDKILQNT | |
| | APKSRNLFERNPNKELKPGENSPRQTPIFDPTVHWLFTTCGASGPHGPTQAQCN | |
| | NAYQNSNLSVEVGSEGPLKGIQIWKVPATDTYSISGYGAAGGKGGKNTMMRSHG | |
| | VSVLGIFNLEKDDMLYILVGQQGEDACPSTNQLIQKVCIGENNVIEEEIRVNRS | |
| | VHEWAGGGGGGGGATYVFKMKDGVPVPLIIAAGGGGRAYGAKTDTFHPERLENN | |
| | SSVLGLNGNSGAAGGGGGWNDNTSLLWAGKSLQEGATGGHSCPQAMKKWGWETR | |
| | GGFGGGGGGCSSGGGGGGYIGGNAASNNDPEMDGEDGVSFISPLGILYTPALKV | |
| | PDVEVKGECSSYYLLLQGNGNRRCKATLIHSANQINGSFALNLIHGKMKTKTEE | |
| | AKLSFPFDLLSLPHFSGEQIVQREKQLANVQVLALEECLKRRKLAKQPETVSVA | |
| | ELKSLLVLTRKHFLDYFDAVIPKMILRKMDKIKTFNILNDFSPVEPNSSSLMET | |
| | NPLEWPERHVLQNLETFEKTKQKMRTGSLPHSSEQLLGHKEGPRDSITLLDAKE | |
| | LLKYFTSDGLPIGDLQPLPIQKGEKTFVLTPELSPGKLQVLPFEKASVCHYHGI | |
| | EYCLDDRKALERDGGFSELQSRLIRYETQTTCTRESFPVPTVLSPLPSPVVSSD | |
| | PGSVPDGEVLQNELRTEVSRLKRRSKDLNCLYPRKRLVKSESSESLLSQTTGNS | |
| | NHYHHHVTSRKPQTERSLPVTCPLVPIPSCETPKLATKTSSGQKSMHESKTSRQ | |
| | IKESRSQKHTRILKEVVTETLKKHSITETHECFTACSQRLFEISKFYLKDLKTS | |
| | RGLFEEMKKTANNNAVQVIDWVLEKTSKK | |
| ALK- | MGAIGLLWLLPLLLSTAAVGSGMGTGQRAGSPAAGPPLQPREPLSYSRLQRKSL | 67 |
| SLC30A6 | AVDFVVPSLFRVYARDLLLPPSSSELKAGRPEARGSLALDCAPLLRLLGPAPGV | |
| | SWTAGSPAPAEARTLSRVLKGGSVRKLRRAKQLVLELGEEAILEGCVGPPGEAA | |
| | VGLLQFNLSELFSWWIRQGEGRLRIRLMPEKKASEVGREGRLSAAIRASQPRLL | |
| | FQIFGTGHSSLESPTNMPSPSPDYFTWNLTWIMKDSFPFLSHRSRYGLECSFDF | |
| | PCELEYSPPLHDLRNQSWSWRRIPSEEASQMDLLDGPGAERSKEMPRGSFLLLN | |
| | TSADSKHTILSPWMRSSSEHCTLAVSVHRHLQPSGRYIAQLLPHNEAAREILLM | |
| | PTPGKHGWTVLQGRIGRPDNPFRVALEYISSGNRSLSAVDFFALKNCSEGTSPG | |
| | SKMALQSSFTCWNGTVLQLGQACDFHQDCAQGEDESQMCRKLPVGFYCNFEDGE | |
| | CGWTQGTLSPHTPQWQVRTLKDARFQDHQDHALLLSTTDVPASESATVTSATFP | |
| | APIKSSPCELRMSWLIRGVLRGNVSLVLVENKTGKEQGRMVWHVAAYEGLSLWQ | |
| | WMVLPLLDVSDREWLQMVAWWGQGSRAIVAFDNISISLDCYLTISGEDKILQNT | |
| | APKSRNLFERNPNKELKPGENSPRQTPIFDPTVHWLFTTCGASGPHGPTQAQCN | |
| | NAYQNSNLSVEVGSEGPLKGIQIWKVPATDTYSISGYGAAGGKGGKNTMMRSHG | |
| | VSVLGIFNLEKDDMLYILVGQQGEDACPSTNQLIQKVCIGENNVIEEEIRVNRS | |
| | VHEWAGGGGGGGGATYVFKMKDGVPVPLIIAAGGGGRAYGAKTDTFHPERLENN | |
| | SSVLGLNGNSGAAGGGGGWNDNTSLLWAGKSLQEGATGGHSCPQAMKKWGWETR | |
| | GGFGGGGGGCSSGGGGGGYIGGNAASNNDPEMDGEDGVSFISPLGILYTPALKV | |
| | MEGHGEVNIKHYLNCSHCEVDECHMDPESHKVICFCDHGTVLAEDGVSCIALTA | |
| | YTYLTIFDLFSLMTCLISYWVTLRKPSPVYSFGFERLEVLAVFASTVLAQLGAL | |
| | FILKESAERFLEQPEIHTGRLLVGTFVALCENLFTMLSIRNKPFAYVSEAASTS | |
| | WLQEHVADLSRSLCGIIPGLSSIFLPRMNPFVLIDLAGAFALCITYMLIEINNY | |
| | FAVDTASAIAIALMTFGTMYPMSVYSGKVLLQTTPPHVIGQLDKLIREVSTLDG | |
| | VLEVRNEHFWTLGFGSLAGSVHVRIRRDANEQMVLAHVTNRLYTLVSTLTVQIF | |
| | KDDWIRPALLSGPVAANVLNFSDHHVIPMPLLKGTDDLNPVTSTPAKPSSPPPE | |
| | FSFNTPGKNVNPVILLNTQTRPYGFGLNHGHTPYSSMLNQGLGVPGIGATQGLR | |
| | TGFTNIPSRYGTNNRIGQPRP | |
| ALK- | MGAIGLLWLLPLLLSTAAVGSGMGTGQRAGSPAAGPPLQPREPLSYSRLQRKSL | 68 |
| GMCL1 | AVDFVVPSLFRVYARDLLLPPSSSELKAGRPEARGSLALDCAPLLRLLGPAPGV | |
| | SWTAGSPAPAEARTLSRVLKGGSVRKLRRAKQLVLELGEEAILEGCVGPPGEAA | |
| | VGLLQFNLSELFSWWIRQGEGRLRIRLMPEKKASEVGREGRLSAAIRASQPRLL | |
| | FQIFGTGHSSLESPTNMPSPSPDYFTWNLTWIMKDSFPFLSHRSRYGLECSEDE | |
| | PCELEYSPPLHDLRNQSWSWRRIPSEEASQMDLLDGPGAERSKEMPRGSFLLLN | |
| | TSADSKHTILSPWMRSSSEHCTLAVSVHRHLQPSGRYIAQLLPHNEAAREILLM | |
| | PTPGKHGWTVLQGRIGRPDNPERVALEYISSGNRSLSAVDFFALKNCSEGTSPG | |
| | SKMALQSSFTCWNGTVLQLGQACDFHQDCAQGEDESQMCRKLPVGFYCNFEDGE | |
| | CGWTQGTLSPHTPQWQVRTLKDARFQDHQDHALLLSTTDVPASESATVTSATFP | |
| | APIKSSPCELRMSWLIRGVLRGNVSLVLVENKTGKEQGRMVWHVAAYEGLSLWQ | |
| | WMVLPLLDVSDREWLQMVAWWGQGSRAIVAFDNISISLDCYLTISGEDKILQNT | |
| | APKSRNLFERNPNKELKPGENSPRQTPIFDPTVHWLFTTCGASGPHGPTQAQCN | |
| | NAYQNSNLSVEVGSEGPLKGIQIWKVPATDTYSISGYGAAGGKGGKNTMMRSHG | |
| | VSVLGIFNLEKDDMLYILVGQQGEDACPSTNQLIQKVCIGENNVIEEEIRVNRS | |
| | VHEWAGGGGGGGGATYVFKMKDGVPVPLIIAAGGGGRAYGAKTDTFHPERLENN | |
| | SSVLGLNGNSGAAGGGGGWNDNTSLLWAGKSLQEGATGGHSCPQAMKKWGWETR | |
| | GGFGGGGGGCSSGGGGGGYIGGNAASNNDPEMDGEDGVSFISPLGILYTPALKV | |
| | MEGHGEVNIKHYLNCSHCEVDECHMDPESHKVICFCDHGTVLAEDGVSCIVSPT | |
| | PEPHLPLSLILSVVTSALVAALVLAFSGIMIVYRRKHQELQAMQMELQSPEYKL | |
| | SKLRTSTIMTDYNPNYCFAGKTSSISDLKEVPRKNITLIRGLGHGAFGEVYEGQ | |
| | VSGMPNDPSPLQVAVKTLPEVCSEQDELDELMEALIISKENHQNIVRCIGVSLQ | |
| | SLPRFILLELMAGGDLKSFLRETRPRPSQPSSLAMLDLLHVARDIACGCQYLEE | |
| | NHFIHRDIAARNCLLTCPGPGRVAKIGDFGMARDIYRASYYRKGGCAMLPVKWM | |
| | PPEAFMEGIFTSKTDTWSFGVLLWEIFSLGYMPYPSKSNQEVLEFVTSGGRMDP | |
| | PKNCPGPVINVMKQLIGSSNLFVMQVEMDIYTALKKWMFLQLVPSWNGSLKQLL | |
| | TETDVWFSKQRKDFEGMAFLETEQGKPFVSVFRHLRLQYIISDLASARIIEQDA | |
| | VVPSEWLSSVYKQQWFAMLRAEQDSEVGPQEINKEELEGNSMRCGRKLAKDGEY | |
| | CWRWTGFNFGFDLLVTYTNRYIIFKRNTLNQPCSGSVSLQPRRSIAFRLRLASF | |
| | DSSGKLICSRTTGYQILTLEKDQEQVVMNLDSRLLIFPLYICCNFLYISPEKKN | |
| ALK- | MGAIGLLWLLPLLLSTAAVGSGMGTGQRAGSPAAGPPLQPREPLSYSRLQRKSL | 69 |
| AGAP1 | AVDFVVPSLFRVYARDLLLPPSSSELKAGRPEARGSLALDCAPLLRLLGPAPGV | |
| | SWTAGSPAPAEARTLSRVLKGGSVRKLRRAKQLVLELGEEAILEGCVGPPGEAA | |
| | VGLLQFNLSELFSWWIRQGEGRLRIRLMPEKKASEVGREGRLSAAIRASQPRLL | |
| | FQIFGTGHSSLESPTNMPSPSPDYFTWNLTWIMKDSFPFLSHRSRYGLECSFDF | |
| | PCELEYSPPLHDLRNQSWSWRRIPSEEASQMDLLDGPGAERSKEMPRGSFLLLN | |
| | TSADSKHTILSPWMRSSSEHCTLAVSVHRHLQPSGRYIAQLLPHNEAAREILLM | |
| | PTPGKHGWTVLQGRIGRPDNPFRVALEYISSGNRSLSAVDFFALKNCSEGTSPG | |
| | SKMALQSSFTCWNGTVLQLGQACDFHQDCAQGEDESQMCRKLPVGFYCNFEDGE | |
| | CGWTQGTLSPHTPQWQVRTLKDARFQDHQDHALLLSTTDVPASESATVTSATFP | |
| | APIKSSPCELRMSWLIRGVLRGNVSLVLVENKTGKEQGRMVWHVAAYEGLSLWQ | |
| | WMVLPLLDVSDREWLQMVAWWGQGSRAIVAFDNISISLDCYLTISGEDKILQNT | |
| | APKSRNLFERNPNKELKPGENSPRQTPIFDPTVHWLFTTCGASGPHGPTQAQCN | |
| | NAYQNSNLSVEVGSEGPLKGIQIWKVPATDTYSISGYGAAGGKGGKNTMMRSHG | |
| | VSVLGIFNLEKDDMLYILVGQQGEDACPSTNQLIQKVCIGENNVIEEEIRVNRS | |
| | VHEWAGGGGGGGGATYVFKMKDGVPVPLIIAAGGGGRAYGAKTDTFHPERLENN | |
| | SSVLGLNGNSGAAGGGGGWNDNTSLLWAGKSLQEGATGGHSCPQAMKKWGWETR | |
| | GGFGGGGGGCSSGGGGGGYIGGNAASNNDPEMDGEDGVSFISPLGILYTPALKV | |
| | MEGHGEVNIKHYLNCSHCEVDECHMDPESHKVICFCDHGTVLAEDGVSCIVSPT | |
| | PEPHLPLSLILSVVTSALVAALVLAFSGIMIVAQKIVATRKKQQLSIGPCKSLP | |
| | NSPSHSSVCSAQVSAVHISQTSNGGGSLSDYSSSVPSTPSTSQKELRIDVPPTA | |
| | NTPTPVRKQSKRRSNLFTSRKGSDPDKEKKGLESRADSIGSGRAIPIKQGMLLK | |
| | RSGKSLNKEWKKKYVTLCDNGVLTYHPSLHDYMQNVHGKEIDLLRTTVKVPGKR | |
| | PPRATSACAPISSPKINGLSKDMSSLHISPNSDTGLGDSVCSSPSISSTTSPKL | |
| | DPPPSPHANRKKHRRKKSTSNFKADGLSGTAEEQEENFEFIIVSLTGQTWHFEA | |
| | TTYEERDAWVQAIESQILASLQSCESSKNKSRLTSQSEAMALQSIRNMRGNSHC | |
| | VDCETQNPNWASLNLGALMCIECSGIHRNLGTHLSRVRSLDLDDWPVELIKVMS | |
| | SIGNELANSVWEESSQGRTKPSVDSTREEKERWIRAKYEQKLFLAPLPCTELSL | |
| | GQHLLRATADEDLRTAILLLAHGSRDEVNETCGEGDGRTALHLACRKGNVVLAQ | |
| | LLIWYGVDVTARDAHGNTALAYARQASSQECIDVLLQYGCPDERFVLMATPNLS | |
| | RRNNNRNNSSGRVPTII | |
| ALK- | MGAIGLLWLLPLLLSTAAVGSGMGTGQRAGSPAAGPPLQPREPLSYSRLQRKSL | 70 |
| ZNF454 | AVDFVVPSLFRVYARDLLLPPSSSELKAGRPEARGSLALDCAPLLRLLGPAPGV | |
| | SWTAGSPAPAEARTLSRVLKGGSVRKLRRAKQLVLELGEEAILEGCVGPPGEAA | |
| | VGLLQFNLSELFSWWIRQGEGRLRIRLMPEKKASEVGREGRLSAAIRASQPRLL | |
| | FQIFGTGHSSLESPTNMPSPSPDYFTWNLTWIMKDSFPFLSHRSRYGLECSFDF | |
| | PCELEYSPPLHDLRNQSWSWRRIPSEEASQMDLLDGPGAERSKEMPRGSFLLLN | |
| | TSADSKHTILSPWMRSSSEHCTLAVSVHRHLQPSGRYIAQLLPHNEAAREILLM | |
| | PTPGKHGWTVLQGRIGRPDNPFRVALEYISSGNRSLSAVDFFALKNCSEGTSPG | |
| | SKMALQSSFTCWNGTVLQLGQACDFHQDCAQGEDESQMCRKLPVGFYCNFEDGF | |
| | CGWTQGTLSPHTPQWQVRTLKDARFQDHQDHALLLSTTDVPASESATVTSATFP | |
| | APIKSSPCELRMSWLIRGVLRGNVSLVLVENKTGKEQGRMVWHVAAYEGLSLWQ | |
| | WMVLPLLDVSDREWLQMVAWWGQGSRAIVAFDNISISLDCYLTISGEDKILQNT | |
| | APKSRNLFERNPNKELKPGENSPRQTPIFDPTVHWLFTTCGASGPHGPTQAQCN | |
| | NAYQNSNLSVEVGSEGPLKGIQIWKVPATDTYSISGYGAAGGKGGKNTMMRSHG | |
| | VSVLGIFNLEKDDMLYILVGQQGEDACPSTNQLIQKVCIGENNVIEEEIRVNRS | |
| | VHEWAGGGGGGGGATYVFKMKDGVPVPLIIAAGGGGRAYGAKTDTFHPERLENN | |
| | SSVLGLNGNSGAAGGGGGWNDNTSLLWAGKSLQEGATGGHSCPQAMKKWGWETR | |
| | GGFGGGGGGCSSGGGGGGYIGGNAASNNDPEMDGEDGVSFISPLGILYTPALKV | |
| | MEGHGEVNIKHYLNCSHCEVDECHMDPESHKVICFCDHGTVLAEDGVSCIVSPT | |
| | PEPHLPLSLILSVVTSALVAALVLAFSGIMIDWMTMPASKKSTVKAEIPEEELD | |
| | QWTIKERFSSSSHWKCASLLEWQCGGQEISLQRVVLTHPNTPSQECDESGSTMS | |
| | SSLHSDQSQGFQPSKNAFECSECGKVFSKSSTLNKHQKIHNEKNANQKIHIKEK | |
| | RYECRECGKAFHQSTHLIHHQRIHTGEKPYECKECGKAFSVSSSLTYHQKIHTG | |
| | EKPFECNLCGKAFIRNIHLAHHHRIHTGEKPFKCNICEKAFVCRAHLTKHQNIH | |
| | SGEKPYKCNECGKAFNQSTSFLQHQRIHTGEKPFECNECGKAFRVNSSLTEHQR | |
| | IHTGEKPYKCNECGKAFRDNSSFARHRKIHTGEKPYRCGLCEKAFRDQSALAQH | |
| | QRIHTGEKPYTCNICEKAFSDHSALTQHKRIHTREKPYKCKICEKAFIRSTHLT | |
| | QHQRIHTGEKPYKCNKCGKAFNQTANLIQHQRHHIGEK | |
| ALK- | MEQSPPPAPEPTQGPTPARSRRRREPESPPASAPTNQLIQKVCIGENNVIEEEI | 71 |
| TTC28 | RVNRSVHEWAGGGGGGGGATYVFKMKDGVPVPLIIAAGGGGRAYGAKTDTFHPE | |
| | RLENNSSVLGLNGNSGAAGGGGGWNDNTSLLWAGKSLQEGATGGHSCPQAMKKW | |
| | GWETRGGFGGGGGGCSSGGGGGGYIGGNAASNNDPEMDGEDGVSFISPLGILYT | |
| | PALKVMEGHGEVNIKHYLNCSHCEVDECHMDPESHKVICFCDHGTVLAEDGVSC | |
| | IVSPTPEPHLPLSLILSVVTSALVAALVLAFSGIMIVYRRKHQELQAMQMELQS | |
| | PEYKLSKLRISTIMTDYNPNYCFAGKTSSISDLKEVPRKNITLIRGLGHGAFGE | |
| | VYEGQVSGMPNDPSPLQVAVKTLPEVCSEQDELDFLMEALIISKENHQNIVRCI | |
| | GVSLQSLPRFILLELMAGGDLKSFLRETRPRPSQPSSLAMLDLLHVARDIACGC | |
| | QYLEENHFIHRDIAARNCLLTCPGPGRVAKIGDFGMARDIYRASYYRKGGCAML | |
| | PVKWMPPEAFMEGIFTSKTDTWSFGVLLWEIFSLGYMPYPSKSNQEVLEFVTSG | |
| | GRMDPPKNCPGPVYRIMTQCWQHQPEDRPNFAIILERIEYCTQDPDVINTALPI | |
| | EYGPLVEEEEKVPVRPKDPEGVPPLLVSQQAKREEERSPAAPPPLPTTSSGKAA | |
| | KKPTAAEISVRVPRGPAVEGGHVNMAFSQSNPPSELHKVHGSRNKPTSLWNPTY | |
| | GSWFTEKPTKKNNPIAKKEPHDRGNLGLEGSCTVPPNVATGRLPGASLLLEPSS | |
| | LTANMKEVPLFRLRHFPCGNVNYGYQQQGLPLEAATAPGAGHYEDTILKSKNSM | |
| | NQPGP | |
| ALK- | MESARENIDLQTNQLIQKVCIGENNVIEEEIRVNRSVHEWAGGGGGGGGATYVF | 72 |
| NINJ2 | KMKDGVPVPLIIAAGGGGRAYGAKTDTFHPERLENNSSVLGLNGNSGAAGGGGG | |
| | WNDNTSLLWAGKSLQEGATGGHSCPQAMKKWGWETRGGFGGGGGGCSSGGGGGG | |
| | YIGGNAASNNDPEMDGEDGVSFISPLGILYTPALKVMEGHGEVNIKHYLNCSHC | |
| | EVDECHMDPESHKVICFCDHGTVLAEDGVSCIVSPTPEPHLPLSLILSVVTSAL | |
| | VAALVLAFSGIMIVYRRKHQELQAMQMELQSPEYKLSKLRTSTIMTDYNPNYCF | |
| | AGKTSSISDLKEVPRKNITLIRGLGHGAFGEVYEGQVSGMPNDPSPLQVAVKTL | |
| | PEVCSEQDELDFLMEALIISKFNHQNIVRCIGVSLQSLPRFILLELMAGGDLKS | |
| | FLRETRPRPSQPSSLAMLDLLHVARDIACGCQYLEENHFIHRDIAARNCLLTCP | |
| | GPGRVAKIGDFGMARDIYRASYYRKGGCAMLPVKWMPPEAFMEGIFTSKTDTWS | |
| | FGVLLWEIFSLGYMPYPSKSNQEVLEFVTSGGRMDPPKNCPGPVYRIMTQCWQH | |
| | QPEDRPNFAIILERIEYCTQDPDVINTALPIEYGPLVEEEEKVPVRPKDPEGVP | |
| | PLLVSQQAKREEERSPAAPPPLPTTSSGKAAKKPTAAEISVRVPRGPAVEGGHV | |
| | NMAFSQSNPPSELHKVHGSRNKPTSLWNPTYGSWFTEKPTKKNNPIAKKEPHDR | |
| | GNLGLEGSCTVPPNVATGRLPGASLLLEPSSLTANMKEVPLFRLRHFPCGNVNY | |
| | GYQQQGLPLEAATAPGAGHYEDTILKSKNSMNQPGP | |
| ALK- | MAKYGEHEASPDNGQNEFSDIIKSRSDEHNDVQKKTFTKWINARFSKSGKPPIN | 73 |
| UTRN | DMFTDLKDGRKLLDLLEGLTGTSLPKERGSTRVHALNNVNRVLQVLHQNNVELV | |
| | NIGGTDIVDGNHKLTLGLLWSIILHWQVKDVMKDVMSDLQQTNSEKILLSWVRQ | |
| | TTRPYSQVNVLNFTTSWTDGLAFNAVLHRHKPDLFSWDKVVKMSPIERLEHAFS | |
| | KAQTYLGIEKLLDPEDVAVQLPDKKSIIMYLTSLFEVLPQQVTIDAIREVETLP | |
| | RKYKKECEEEAINIQSTAPEEEHESPRAETPSTVTEVDMDLDSYQIALEEVLTW | |
| | LLSAEDTFQEQDDISDDVEEVKDQFATHEAFMMELTAHQSSVGSVLQAGNQLIT | |
| | QGTLSDEEEFEIQEQMTLLNARWEALRVESMDRQSRLHDVLMELQKKQLQQLSA | |
| | WLTLTEERIQKMETCPLDDDVKSLQKLLEEHKSLQSDLEAEQVKVNSLTHMVVI | |
| | VDENSGESATAILEDQLQKLGERWTAVCRWTEERWNRLQEINILWQELLEEQCL | |
| | LKAWLTEKEEALNKVQTSNFKDQKELSVSVRRLAILKEDMEMKRQTLDQLSEIG | |
| | QDVGQLLDNSKASKKINSDSEELTQRWDSLVQRLEDSSNQVTQAVAKLGMSQIP | |
| | QKDLLETVRVREQAITKKSKQELPPPPPPKKRQIHVDIEAKKKFDAISAELLNW | |
| | ILKWKTAIQTTEIKEYMKMQDTSEMKKKLKALEKEQRERIPRADELNQTGQILV | |
| | EQMGKEGLPTEEIKNVLEKVSSEWKNVSQHLEDLERKIQLQEDINAYFKQLDEL | |
| | EKVIKTKEEWVKHTSISESSRQSLPSLKDSCQRELTNLLGLHPKIEMARASCSA | |
| | LMSQPSAPDFVQRGFDSFLGRYQAVQEAVEDRQQHLENELKGQPGHAYLETLKT | |
| | LKDVLNDSENKAQVSLNVLNDLAKVEKALQEKKILDEILENQKPALHKLAEETK | |
| | ALEKNVHPDVEKLYKQEFDDVQGKWNKLKVLVSKDLHLLEEIALTLRAFEADST | |
| | VIEKWMDGVKDFLMKQQAAQGDDAGLQRQLDQCSAFVNEIETIESSLKNMKEIE | |
| | TNLRSGPVAGIKTWVQTRLGDYQTQLEKLSKEIATQKSRLSESQEKAANLKKDL | |
| | AEMQEWMTQAEEEYLERDFEYKSPEELESAVEEMKRAKEDVLQKEVRVKILKDN | |
| | IKLLAAKVPSGGQELTSELNVVLENYQLLCNRIRGKCHTLEEVWSCWIELLHYL | |
| | DLETTWLNTLEERMKSTEVLPEKTDAVNEALESLESVLRHPADNRTQIRELGQT | |
| | LIDGGILDDIISEKLEAFNSRYEDLSHLAESKQISLEKQLQVLRETDQMLQVLQ | |
| | ESLGELDKQLTTYLTDRIDAFQVPQEAQKIQAEISAHELTLEELRRNMRSQPLT | |
| | SPESRTARGGSQMDVLQRKLREVSTKFQLFQKPANFEQRMLDCKRVLDGVKAEL | |
| | HVLDVKDVDPDVIQTHLDKCMKLYKTLSEVKLEVETVIKTGRHIVQKQQTDNPK | |
| | GMDEQLTSLKVLYNDLGAQVTEGKQDLERASQLARKMKKEAASLSEWLSATETE | |
| | LVQKSTSEGLLGDLDTEISWAKNVLKDLEKRKADLNTITESSAALQNLIEGSEP | |
| | ILEERLCVLNAGWSRVRTWTEDWCNTLMNHQNQLEIFDGNVAHISTWLYQAEAL | |
| | LDEIEKKPTSKQEEIVKRLVSELDDANLQVENVRDQALILMNARGSSSRELVEP | |
| | KLAELNRNFEKVSQHIKSAKLLIAQEPLYQCLVTTETFETGVPFSDLEKLENDI | |
| | ENMLKFVEKHLESSDEDEKMDEESAQIEEVLQRGEEMLHQPMEDNKKEKIRLQL | |
| | LLLHTRYNKIKAIPIQQRKMGQLASGIRSSLLPTDYLVEINKILLCMDDVELSL | |
| | NVPELNTAIYEDFSFQEDSLKNIKDQLDKLGEQIAVIHEKQPDVILEASGPEAI | |
| | QIRDTLTQLNAKWDRINRMYSDRKGCFDRAMEEWRQFHCDLNDLTQWITEAEEL | |
| | LVDTCAPGGSLDLEKARIHQQELEVGISSHQPSFAALNRTGDGIVQKLSQADGS | |
| | FLKEKLAGLNQRWDAIVAEVKDRQPRLKGESKQVMKYRHQLDEIICWLTKAEHA | |
| | MQKRSTTELGENLQELRDLTQEMEVHAEKLKWLNRTELEMLSDKSLSLPERDKI | |
| | SESLRTVNMTWNKICREVPTTLKECIQEPSSVSQTRIAAHPNVQKVVLVSSASD | |
| | IPVQSHRTSEISIPADLDKTITELADWLVLIDQMLKSNIVTVGDVEEINKTVSR | |
| | MKITKADLEQRHPQLDYVFTLAQNLKNKASSSDMRTAITEKLERVKNQWDGTQH | |
| | GVELRQQQLEDMIIDSLQWDDHREETEELMRKYEARLYILQQARRDPLTKQISD | |
| | NQILLQELGPGDGIVMAFDNVLQKLLEEYGSDDTRNVKETTEYLKTSWINLKQS | |
| | IADRQNALEAEWRTVQASRRDLENFLKWIQEAETTVNVLVDASHRENALQDSIL | |
| | ARELKQQMQDIQAEIDAHNDIFKSIDGNRQKMVKALGNSEEATMLQHRLDDMNQ | |
| | RWNDLKAKSASIRAHLEASAEKWNRLLMSLEELIKWLNMKDEELKKQMPIGGDV | |
| | PALQLQYDHCKALRRELKEKEYSVLNAVDQARVFLADQPIEAPEEPRRNLQSKT | |
| | ELTPEERAQKIAKAMRKQSSEVKEKWESLNAVTSNWQKQVDKALEKLRDLQGAM | |
| | DDLDADMKEAESVRNGWKPVGDLLIDSLQDHIEKIMAFREEIAPINFKVKTVND | |
| | LSSQLSPLDLHPSLKMSRQLDDLNMRWKLLQVSVDDRLKQLQEAHRDFGPSSQH | |
| | FLSTSVQLPWQRSISHNKVPYYINHQTQTTCWDHPKMTELFQSLADLNNVRESA | |
| | YRTAIKIRRLQKALCLDLLELSTTNEIFKQHKLNQNDQLLSVPDVINCLTTTYD | |
| | GLEQMHKDLVNVPLCVDMCLNWLLNVYDTGRTGKIRVQSLKIGLMSLSKGLLEE | |
| | KYRYLFKEVAGPTEMCDQRQLGLLLHDAIQIPRQLGEVAAFGGSNIEPSVRSCF | |
| | QQNNNKPEISVKEFIDWMHLEPQSMVWLPVLHRVAAAETAKHQAKCNICKECPI | |
| | VGFRYRSLKHFNYDVCQSCFFSGRTAKGHKLHYPMVEYCIPTTSGEDVRDFTKV | |
| | LKNKFRSKKYFAKHPRLGYLPVQTVLEGDNLETPITLISMWPEHYDPSQSPQLF | |
| | HDDTHSRIEQYATRLAQMERINGSFLTDSSSTTGSVEDEHALIQQYCQTLGGES | |
| | PVSQPQSPAQILKSVEREERGELERIIADLEEEQRNLQVEYEQLKDQHLRRGLP | |
| | VGSPPESIISPHHTSEDSELIAEAKLLRQHKGRLEARMQILEDHNKQLESQLHR | |
| | LRQLLEQPESDSRINGVSPWASPQHSALSYSLDPDASGPQFHQAVSPTPEPHLP | |
| | LSLILSVVTSALVAALVLAFSGIMIVYRRKHQELQAMQMELQSPEYKLSKIRTS | |
| | TIMTDYNPNYCFAGKTSSISDLKEVPRKNITLIRGLGHGAFGEVYEGQVSGMPN | |
| | DPSPLQVAVKTLPEVCSEQDELDELMEALIISKENHQNIVRCIGVSLQSLPRFI | |
| | LLELMAGGDLKSFLRETRPRPSQPSSLAMLDLLHVARDIACGCQYLEENHFIHR | |
| | DIAARNCLLTCPGPGRVAKIGDFGMARDIYRASYYRKGGCAMLPVKWMPPEAFM | |
| | EGIFTSKTDTWSFGVLLWEIFSLGYMPYPSKSNQEVLEFVTSGGRMDPPKNCPG | |
| | PVYRIMTQCWQHQPEDRPNFAIILERIEYCTQDPDVINTALPIEYGPLVEEEEK | |
| | VPVRPKDPEGVPPLLVSQQAKREEERSPAAPPPLPTTSSGKAAKKPTAAEISVR | |
| | VPRGPAVEGGHVNMAFSQSNPPSELHKVHGSRNKPTSLWNPTYGSWFTEKPTKK | |
| | NNPIAKKEPHDRGNLGLEGSCTVPPNVATGRLPGASLLLEPSSLTANMKEVPLF | |
| | RLRHFPCGNVNYGYQQQGLPLEAATAPGAGHYEDTILKSKNSMNQPGP | |
| ALK- | MVDYHAANQSYQYGPSSAGNGAGGGGSMGDYMAQEDDWDRDLLLDPAWEKQQRK | 74 |
| ACTN4 | TFTAWCNSHLRKAGTQIENIDEDFRDGLKLMLLLEVISGERLPKPERGKMRVHK | |
| | INNVNKALDFIASKGVKLVSIGAEEIVDGNAKMTLGMIWTIILRFAIQDISVEE | |
| | TSAKEGLLLWCQRKTAPYKNVNVQNFHISWKDGLAFNALIHRHRPELIEYDKLR | |
| | KDDPVTNLNNAFEVAEKYLDIPKMLDAEVYRRKHQELQAMQMELQSPEYKLSKL | |
| | RTSTIMTDYNPNYCFAGKTSSISDLKEVPRKNITLIRGLGHGAFGEVYEGQVSG | |
| | MPNDPSPLQVAVKTLPEVCSEQDELDFLMEALIISKFNHQNIVRCIGVSLQSLP | |
| | RFILLELMAGGDLKSFLRETRPRPSQPSSLAMLDLLHVARDIACGCQYLEENHF | |
| | IHRDIAARNCLLTCPGPGRVAKIGDFGMARDIYRASYYRKGGCAMLPVKWMPPE | |
| | AFMEGIFTSKTDTWSFGVLLWEIFSLGYMPYPSKSNQEVLEFVTSGGRMDPPKN | |
| | CPGPVYRIMTQCWQHQPEDRPNFAIILERIEYCTQDPDVINTALPIEYGPLVEE | |
| | EEKVPVRPKDPEGVPPLLVSQQAKREEERSPAAPPPLPTTSSGKAAKKPTAAEI | |
| | SVRVPRGPAVEGGHVNMAFSQSNPPSELHKVHGSRNKPTSLWNPTYGSWFTEKP | |
| | TKKNNPIAKKEPHDRGNLGLEGSCTVPPNVATGRLPGASLLLEPSSLTANMKEV | |
| | PLFRLRHFPCGNVNYGYQQQGLPLEAATAPGAGHYEDTILKSKNSMNQPGP | |
| ALK- | MGRPESAGGGSRGPFEGGGRARRAGGIFLTLSILRTRDLPSGAMSEGVDLIDIY | 75 |
| CPSF7 | ADEEFNQDPEFNNTDQIDLYDDVLTATSQPSDDRSSSTEPPPPVRQEPSPKPNN | |
| | KTPAILYTYSGLRNRRAAVYVGSFSWWTTDQQLIQVIRSIGVYDVVELKFAENR | |
| | ANGQSKGYAEVVVASENSVHKLLELLPGKVLNGEKVDVRPATRQNLSQFEAQAR | |
| | KRECVRVPRGVYRRKHQELQAMQMELQSPEYKLSKLRISTIMTDYNPNYCFAGK | |
| | TSSISDLKEVPRKNITLIRGLGHGAFGEVYEGQVSGMPNDPSPLQVAVKTLPEV | |
| | CSEQDELDELMEALIISKENHQNIVRCIGVSLQSLPRFILLELMAGGDLKSFLR | |
| | ETRPRPSQPSSLAMLDLLHVARDIACGCQYLEENHFIHRDIAARNCLLTCPGPG | |
| | RVAKIGDFGMARDIYRASYYRKGGCAMLPVKWMPPEAFMEGIFTSKTDTWSFGV | |
| | LLWEIFSLGYMPYPSKSNQEVLEFVTSGGRMDPPKNCPGPVYRIMTQCWQHQPE | |
| | DRPNFAIILERIEYCTQDPDVINTALPIEYGPLVEEEEKVPVRPKDPEGVPPLL | |
| | VSQQAKREEERSPAAPPPLPTTSSGKAAKKPTAAEISVRVPRGPAVEGGHVNMA | |
| | FSQSNPPSELHKVHGSRNKPTSLWNPTYGSWFTEKPTKKNNPIAKKEPHDRGNL | |
| | GLEGSCTVPPNVATGRLPGASLLLEPSSLTANMKEVPLFRLRHFPCGNVNYGYQ | |
| | QQGLPLEAATAPGAGHYEDTILKSKNSMNQPGP | |
| ALK- | MAAAKVALTKRADPAELRTIFLKYASIEKNGEFFMSPNDFVTRYLNIFGESQPN | 76 |
| SLC25A13 | PKTVELLSGVVDQTKDGLISFQEFVAFESVLCAPDALFMVAFQLEDKAGKGEVT | |
| | FEDVKQVFGQTTIHQHIPFNWDSEFVQLHFGKERKRHLTYAEFTQFLLEIQLEH | |
| | AKQAFVQRDNARTGRVTAIDERDIMVTIRPHVLTPFVEECLVAAAGGTTSHQVS | |
| | FSYFNGFNSLLNNMELIRKIYSTLAGTRKDVEVTKEEFVLAAQKFGQVTPMEVD | |
| | ILFQLADLYEPRGRMTLADIERIAPLEEGTLPFNLAEAQRQKASGDSARPVLLQ | |
| | VAESAYRFGLGSVAGVYRRKHQELQAMQMELQSPEYKLSKLRISTIMTDYNPNY | |
| | CFAGKTSSISDLKEVPRKNITLIRGLGHGAFGEVYEGQVSGMPNDPSPLQVAVK | |
| | TLPEVCSEQDELDFLMEALIISKFNHQNIVRCIGVSLQSLPRFILLELMAGGDL | |
| | KSFLRETRPRPSQPSSLAMLDLLHVARDIACGCQYLEENHFIHRDIAARNCLLT | |
| | CPGPGRVAKIGDFGMARDIYRASYYRKGGCAMLPVKWMPPEAFMEGIFTSKTDT | |
| | WSFGVLLWEIFSLGYMPYPSKSNQEVLEFVTSGGRMDPPKNCPGPVYRIMTQCW | |
| | QHQPEDRPNFAIILERIEYCTQDPDVINTALPIEYGPLVEEEEKVPVRPKDPEG | |
| | VPPLLVSQQAKREEERSPAAPPPLPTTSSGKAAKKPTAAEISVRVPRGPAVEGG | |
| | HVNMAFSQSNPPSELHKVHGSRNKPTSLWNPTYGSWFTEKPTKKNNPIAKKEPH | |
| | DRGNLGLEGSCTVPPNVATGRLPGASLLLEPSSLTANMKEVPLFRLRHFPCGNV | |
| | NYGYQQQGLPLEAATAPGAGHYEDTILKSKNSMNQPGP | |
| ALK- | MDDSEVESTASILASVKEQEAQFEKLTRALEEERRHVSAQLERVRVSPQDANPL | 77 |
| CTNND1 | MANGTLTRRHQNGRFVGDADLERQKFSDLKLNGPQDHSHLLYSTIPRMQEPGQI | |
| | VETYTEEDPEGAMSVVSVETSDDGTTRRTETTVKKVVKTVTTRTVQPVAMGPDG | |
| | LPVDASSVSNNYIQTLGRDFRKNGNGGPGPYVGQAGTATLPRNFHYPPDGYSRH | |
| | YEDGYPGGSDNYGSLSRVTRIEERYRPSMEGYRAPSRQDVYGPQPQVRVGGSSV | |
| | DLHRFHPEPYGLEDDQRSMGYDDLDYGMMSDYGTARRTGTPSDPRRRLRSYEDM | |
| | IGEEVPSDQYYWAPLAQHERGSLASLDSLRKGGPPPPNWRQPELPEVIAMLGER | |
| | LDAVKSNAAAYLQHLCYRNDKVKTDVRKLKGIPVLVGLLDHPKKEVHLGACGAL | |
| | KNISFGRDQDNKIAIKNCDGVPALVRLLRKARDMDLTEVITGTLWNLSSHDSIK | |
| | MEIVDHALHALTDEVIIPHSGWEREPNEDCKPRHIEWESVLINTAGCLRNVSSE | |
| | RSEARRKLRECDGLVDALIFIVQAEIGQKDSDSKLVENCVCLLRNLSYQVHREI | |
| | PQAERYQEAAPNVANNTGPHAASCFGAKKGKGKKPIEDPANDTVDFPKRISPAR | |
| | GYELLFQPEVVRIYISLLKESKTPAILEASAGAIQNLCAGRWTYGRYIRSALRQ | |
| | EKALSAIADLLTNEHERVVKAASGALRNLAVDARNKELIGKHAIPNLVKNLPGG | |
| | QQNSSWNFSEDTVISILNTINEVIAENLEAAKKLRETQGIEKLVLINKSGNRSE | |
| | KEVRAAALVLQTIWGYKELRKPLEKEGWKKSDFQVNLNNASRSQSSHSYDDSTL | |
| | PLIDRNQKSVYRRKHQELQAMQMELQSPEYKLSKLRTSTIMTDYNPNYCFAGKT | |
| | SSISDLKEVPRKNITLIRGLGHGAFGEVYEGQVSGMPNDPSPLQVAVKTLPEVC | |
| | SEQDELDELMEALIISKENHQNIVRCIGVSLQSLPRFILLELMAGGDLKSFLRE | |
| | TRPRPSQPSSLAMLDLLHVARDIACGCQYLEENHFIHRDIAARNCLLTCPGPGR | |
| | VAKIGDFGMARDIYRASYYRKGGCAMLPVKWMPPEAFMEGIFTSKIDTWSFGVL | |
| | LWEIFSLGYMPYPSKSNQEVLEFVTSGGRMDPPKNCPGPVYRIMTQCWQHQPED | |
| | RPNFAIILERIEYCTQDPDVINTALPIEYGPLVEEEEKVPVRPKDPEGVPPLLV | |
| | SQQAKREEERSPAAPPPLPTTSSGKAAKKPTAAEISVRVPRGPAVEGGHVNMAF | |
| | SQSNPPSELHKVHGSRNKPTSLWNPTYGSWFTEKPTKKNNPIAKKEPHDRGNLG | |
| | LEGSCTVPPNVATGRLPGASLLLEPSSLTANMKEVPLFRLRHFPCGNVNYGYQQ | |
| | QGLPLEAATAPGAGHYEDTILKSKNSMNQPGP | |
| ALK-KLC4 | MSGLVLGQRDEPAGHRLSQEEILGSTRLVSQGLEALRSEHQAVLQSLSQTIECL | 78 |
| | QQGGHEEGLVHEKARQLRRSMENIELGLSEAQVMLALASHLSTVESEKQKLRAQ | |
| | VRRLCQENQWLRDELAGTQQRLQRSEQAVAQLEEEKKHLEFLGQLRQYDEDGHT | |
| | SEEKEGDATKDSLDDLFPNEEEEDPSNGLSRGQGATAAQQGGYEIPARLRTLHN | |
| | LVIQYAAQGRYEVAVPLCKQALEDLERTSGRGHPDVATMLNILALVYRDQNKYK | |
| | EAAHLLNDALSIRESTLGPDHPAVAATLNNLAVLYGKRGKYKEAEPLCQRALEI | |
| | REKVLGTNHPDVAKQLNNLALLCQNQGKYEAVERYYQRALAIYEGQLGPDNPNV | |
| | ARTKNNLASCYLKQGKYAEAETLYKEILTRAHVQEFGSVDVYRRKHQELQAMQM | |
| | ELQSPEYKLSKLRISTIMTDYNPNYCFAGKTSSISDLKEVPRKNITLIRGLGHG | |
| | AFGEVYEGQVSGMPNDPSPLQVAVKTLPEVCSEQDELDELMEALIISKENHQNI | |
| | VRCIGVSLQSLPRFILLELMAGGDLKSFLRETRPRPSQPSSLAMLDLLHVARDI | |
| | ACGCQYLEENHFIHRDIAARNCLLTCPGPGRVAKIGDFGMARDIYRASYYRKGG | |
| | CAMLPVKWMPPEAFMEGIFTSKTDTWSFGVLLWEIFSLGYMPYPSKSNQEVLEF | |
| | VTSGGRMDPPKNCPGPVYRIMTQCWQHQPEDRPNFAIILERIEYCTQDPDVINT | |
| | ALPIEYGPLVEEEEKVPVRPKDPEGVPPLLVSQQAKREEERSPAAPPPLPTTSS | |
| | GKAAKKPTAAEISVRVPRGPAVEGGHVNMAFSQSNPPSELHKVHGSRNKPTSLW | |
| | NPTYGSWFTEKPTKKNNPIAKKEPHDRGNLGLEGSCTVPPNVATGRLPGASLLL | |
| | EPSSLTANMKEVPLFRLRHFPCGNVNYGYQQQGLPLEAATAPGAGHYEDTILKS | |
| | KNSMNQPGP | |
| ALK- | MSNSHPLRPFTAVGEIDHVHILSEHIGALLIGEEYGDVTFVVEKKRFPAHRVIL | 79 |
| BTBD9 | AARCQYFRALLYGGMRESQPEAEIPLQDTTAEAFTMLLKYIYTGRATLTDEKEE | |
| | VLLDFLSLAHKYGFPELEDSTSEYLCTILNIQNVCMTFDVASLYSLPKLTCMCC | |
| | MFMDRNAQEVLSSEGFLSLSKTALLNIVLRDSFAAPEKDIFLALLNWCKANSKE | |
| | NHAEIMQAVRLPLMSLTELLNVVRPSGLLSPDAILDAIKVRSESRDMDLNYRGM | |
| | LMYRRKHQELQAMQMELQSPEYKLSKLRISTIMTDYNPNYCFAGKTSSISDLKE | |
| | VPRKNITLIRGLGHGAFGEVYEGQVSGMPNDPSPLQVAVKTLPEVCSEQDELDF | |
| | LMEALIISKFNHQNIVRCIGVSLQSLPRFILLELMAGGDLKSFLRETRPRPSQP | |
| | SSLAMLDLLHVARDIACGCQYLEENHFIHRDIAARNCLLTCPGPGRVAKIGDFG | |
| | MARDIYRASYYRKGGCAMLPVKWMPPEAFMEGIFTSKIDTWSFGVLLWEIFSLG | |
| | YMPYPSKSNQEVLEFVTSGGRMDPPKNCPGPVYRIMTQCWQHQPEDRPNFAIIL | |
| | ERIEYCTQDPDVINTALPIEYGPLVEEEEKVPVRPKDPEGVPPLLVSQQAKREE | |
| | ERSPAAPPPLPTTSSGKAAKKPTAAEISVRVPRGPAVEGGHVNMAFSQSNPPSE | |
| | LHKVHGSRNKPTSLWNPTYGSWFTEKPTKKNNPIAKKEPHDRGNLGLEGSCTVP | |
| | PNVATGRLPGASLLLEPSSLTANMKEVPLFRLRHFPCGNVNYGYQQQGLPLEAA | |
| | TAPGAGHYEDTILKSKNSMNQPGP | |
| ALK-CPQ | MKFLIFAFFGGVHLLSLCSGKAICKNGISKRTFEEIKEEIASCGDVAKAIINLA | 81 |
| | VYGKAQNRSYERLALLVDTVGPRLSGSKNLEKAIQIMYQNLQQDGLEKVHLEPV | |
| | RIPHWERGEESAVMLEPRIHKIAILGLGSSIGTPPEGITAEVLVVTSFDELQRR | |
| | ASEARGKIVVYNQPYINYSRTVQYRTQGAVEAAKVGALASLIRSVASFSIYSPH | |
| | TGIQEYQDGVPKIPTACITVEDAEMMSRMASHGIKIVIQLKMGAKTYPDTDSEN | |
| | TVAEITGSKYPEQVVLVSGHLDSWDVGQGAMDDGGGAFISWEALSLIKDLGLRP | |
| | KRTLRLVLWTAEEQGGVGAFQYYQLHKVNISNYSLVMESDAGTFLPTGLQFTGS | |
| | EKARAIMEEVMSLLQPLNITQVLSHGEGTDINFWIQAGVPVYRRKHQELQAMQM | |
| | ELQSPEYKLSKLRISTIMTDYNPNYCFAGKTSSISDLKEVPRKNITLIRGLGHG | |
| | AFGEVYEGQVSGMPNDPSPLQVAVKTLPEVCSEQDELDELMEALIISKENHQNI | |
| | VRCIGVSLQSLPRFILLELMAGGDLKSFLRETRPRPSQPSSLAMLDLLHVARDI | |
| | ACGCQYLEENHFIHRDIAARNCLLTCPGPGRVAKIGDFGMARDIYRASYYRKGG | |
| | CAMLPVKWMPPEAFMEGIFTSKTDTWSFGVLLWEIFSLGYMPYPSKSNQEVLEF | |
| | VTSGGRMDPPKNCPGPVYRIMTQCWQHQPEDRPNFAIILERIEYCTQDPDVINT | |
| | ALPIEYGPLVEEEEKVPVRPKDPEGVPPLLVSQQAKREEERSPAAPPPLPTTSS | |
| | GKAAKKPTAAEISVRVPRGPAVEGGHVNMAFSQSNPPSELHKVHGSRNKPTSLW | |
| | NPTYGSWFTEKPTKKNNPIAKKEPHDRGNLGLEGSCTVPPNVATGRLPGASLLL | |
| | EPSSLTANMKEVPLFRLRHFPCGNVNYGYQQQGLPLEAATAPGAGHYEDTILKS | |
| | KNSMNQPGP | |
| ALK- | MADPAECSIKVMCRFRPLNEAEILRGDKFIPKFKGDETVVIGQGKPYVFDRVLP | 83 |
| KIF5C | PNTTQEQVYNACAKQIVKDVLEGYNGTIFAYGQTSSGKTHTMEGKLHDPQLMGI | |
| | IPRIAHDIFDHIYSMDENLEFHIKVSYFEIYLDKIRDLLDVSKINLAVHEDKNR | |
| | VPYVKGCTERFVSSPEEVMDVIDEGKANRHVAVINMNEHSSRSHSIFLINIKQE | |
| | NVETEKKLSGKLYLVDLAGSEKVSKTGAEGAVLDEAKNINKSLSALGNVISALA | |
| | EGTKTHVPYRDSKMTRILQDSLGGNCRTTIVICCSPSVENEAETKSTLMFGQRA | |
| | KTIKNTVSVNLELTAEEWKKKYEKEKEKNKTLKNVIQHLEMELNRWRNVYRRKH | |
| | QELQAMQMELQSPEYKLSKLRISTIMTDYNPNYCFAGKTSSISDLKEVPRKNIT | |
| | LIRGLGHGAFGEVYEGQVSGMPNDPSPLQVAVKTLPEVCSEQDELDELMEALII | |
| | SKFNHQNIVRCIGVSLQSLPRFILLELMAGGDLKSFLRETRPRPSQPSSLAMLD | |
| | LLHVARDIACGCQYLEENHFIHRDIAARNCLLTCPGPGRVAKIGDFGMARDIYR | |
| | ASYYRKGGCAMLPVKWMPPEAFMEGIFTSKTDTWSFGVLLWEIFSLGYMPYPSK | |
| | SNQEVLEFVTSGGRMDPPKNCPGPVYRIMTQCWQHQPEDRPNFAIILERIEYCT | |
| | QDPDVINTALPIEYGPLVEEEEKVPVRPKDPEGVPPLLVSQQAKREEERSPAAP | |
| | PPLPTTSSGKAAKKPTAAEISVRVPRGPAVEGGHVNMAFSQSNPPSELHKVHGS | |
| | RNKPTSLWNPTYGSWFTEKPTKKNNPIAKKEPHDRGNLGLEGSCTVPPNVATGR | |
| | LPGASLLLEPSSLTANMKEVPLFRLRHFPCGNVNYGYQQQGLPLEAATAPGAGH | |
| | YEDTILKSKNSMNQPGP | |
| ALK- | MAVASDFYLRYYVGHKGKFGHEFLEFEFRPDVYRRKHQELQAMQMELQSPEYKL | 84 |
| MAGOHB | SKLRTSTIMTDYNPNYCFAGKTSSISDLKEVPRKNITLIRGLGHGAFGEVYEGQ | |
| | VSGMPNDPSPLQVAVKTLPEVCSEQDELDELMEALIISKENHQNIVRCIGVSLQ | |
| | SLPRFILLELMAGGDLKSFLRETRPRPSQPSSLAMLDLLHVARDIACGCQYLEE | |
| | NHFIHRDIAARNCLLTCPGPGRVAKIGDFGMARDIYRASYYRKGGCAMLPVKWM | |
| | PPEAFMEGIFTSKTDTWSFGVLLWEIFSLGYMPYPSKSNQEVLEFVTSGGRMDP | |
| | PKNCPGPVYRIMTQCWQHQPEDRPNFAIILERIEYCTQDPDVINTALPIEYGPL | |
| | VEEEEKVPVRPKDPEGVPPLLVSQQAKREEERSPAAPPPLPTTSSGKAAKKPTA | |
| | AEISVRVPRGPAVEGGHVNMAFSQSNPPSELHKVHGSRNKPTSLWNPTYGSWFT | |
| | EKPTKKNNPIAKKEPHDRGNLGLEGSCTVPPNVATGRLPGASLLLEPSSLTANM | |
| | KEVPLFRLRHFPCGNVNYGYQQQGLPLEAATAPGAGHYEDTILKSKNSMNQPGP | |
| ALK- | MMSFVQKGSWLLLALLHPTIILAQQEAVEGGCSHLGQSYADRDVWKPEPCQICV | 86 |
| COL3A1 | CDSGSVLCDDIICDDQELDCPNPEIPFGECCAVCPQPPTAPTRPPNGQGPQGPK | |
| | GDPGPPGIPGRNGDPGIPGQPGSPGSPGPPGICESCPTGPQNYSPQYDSYDVKS | |
| | GVAVGGLAGYPGPAGPPGPPGPPGTSGHPGSPGSPGYQGPPGEPGQAGPSGPPG | |
| | PPGAIGPSGPAGKDGESGRPGRPGERGLPGPPGIKGPAGIPGFPGMKGHRGFDG | |
| | RNGEKGETGAPGLKGENGLPGENGAPGPMGPRGAPGERGRPGLPGAAGARGNDG | |
| | ARGSDGQPGPPGPPGTAGFPGSPGAKGEVGPAGSPGSNGAPGQRGEPGPQGHAG | |
| | AQGPPGPPGINGSPGGKGEMGPAGIPGAPGLMGARGPPGPAGANGAPGLRGGAG | |
| | EPGKNGAKGEPGPRGERGEAGIPGVPGAKGEDGKDGSPGEPGANGLPGAAGERG | |
| | APGFRGPAGPNGIPGEKGPAGERGAPGPAGPRGAAGEPGRDGVPGGPGMRGMPG | |
| | SPGGPGSDGKPGPPGSQGESGRPGPPGPSGPRGQPGVMGFPGPKGNDGAPGKNG | |
| | ERGGPGGPGPQGPPGKNGETGPQGPPGPTGPGGDKGDTGPPGPQGLQGLPGTGG | |
| | PPGENGKPGEPGPKGDAGAPGAPGGKGDAGAPGERGPPGLAGAPGLRGGAGPPG | |
| | PEGGKGAAGPPGPPGAAGTPGLQGMPGERGGLGSPGPKGDKGEPGGPGADGVPG | |
| | KDGPRGPTGPIGPPGPAGQPGDKGEGGAPGLPGIAGPRGSPGERGETGPPGPAG | |
| | FPGAPGQNGEPGGKGERGAPGEKGEGGPPGVAGPPGGSGPAGPPGPQGVKGERG | |
| | SPGGPGAAGFPGARGLPGPPGSNGNPGPPGPSGSPGKDGPPGPAGNTGAPGSPG | |
| | VSGPKGDAGQPGEKGSPGAQGPPGAPGPLGIAGITGARGLAGPPGMPGPRGSPG | |
| | PQGVKGESGKPGANGLSGERGPPGPQGLPGLAGTAGEPGRDGNPGSDGLPGRDG | |
| | SPGGKGDRGENGSPGAPGAPGHPGPPGPVGPAGKSGDRGESGPAGPAGAPGPAG | |
| | SRGAPGPQGPRGDKGETGERGAAGIKGHRGFPGNPGAPGSPGPAGQQGAIGSPG | |
| | PAGPRGPVGPSGPPGKDGTSGHPGPIGPPGPRGNRGERGSEGSPGHPGQPGPPG | |
| | PPGAPGPCCGGVGAAAIAGIGGEKAGGFAPYYGDEPMDFKINTDEIMTSLKSVN | |
| | GQIESLISPDGSRKNPARNCRDLKFCHPELKSVSPTPEPHLPLSLILSVVTSAL | |
| | VAALVLAFSGIMIVYRRKHQELQAMQMELQSPEYKLSKLRTSTIMTDYNPNYCF | |
| | AGKTSSISDLKEVPRKNITLIRGLGHGAFGEVYEGQVSGMPNDPSPLQVAVKTL | |
| | PEVCSEQDELDELMEALIISKENHQNIVRCIGVSLQSLPRFILLELMAGGDLKS | |
| | FLRETRPRPSQPSSLAMLDLLHVARDIACGCQYLEENHFIHRDIAARNCLLTCP | |
| | GPGRVAKIGDFGMARDIYRASYYRKGGCAMLPVKWMPPEAFMEGIFTSKTDTWS | |
| | FGVLLWEIFSLGYMPYPSKSNQEVLEFVTSGGRMDPPKNCPGPVYRIMTQCWQH | |
| | QPEDRPNFAIILERIEYCTQDPDVINTALPIEYGPLVEEEEKVPVRPKDPEGVP | |
| | PLLVSQQAKREEERSPAAPPPLPTTSSGKAAKKPTAAEISVRVPRGPAVEGGHV | |
| | NMAFSQSNPPSELHKVHGSRNKPTSLWNPTYGSWFTEKPTKKNNPIAKKEPHDR | |
| | GNLGLEGSCTVPPNVATGRLPGASLLLEPSSLTANMKEVPLFRLRHFPCGNVNY | |
| | GYQQQGLPLEAATAPGAGHYEDTILKSKNSMNQPGP | |

In some embodiments, the ALK-COL3A1 fusion polypeptide of the disclosure comprises the amino acid sequence of SEQ ID NO: 44, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-CDC42BPA fusion polypeptide of the disclosure comprises the amino acid sequence of SEQ ID NO: 45, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-EPHA2 fusion polypeptide of the disclosure comprises the amino acid sequence of SEQ ID NO: 46, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MYOSC fusion polypeptide of the disclosure comprises the amino acid sequence of SEQ ID NO: 47, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TRIM24 fusion polypeptide of the disclosure comprises the amino acid sequence of SEQ ID NO: 48, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-SKAP1 fusion polypeptide of the disclosure comprises the amino acid sequence of SEQ ID NO: 49, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-UBE3B fusion polypeptide of the disclosure comprises the amino acid sequence of SEQ ID NO: 50, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TNS3 fusion polypeptide of the disclosure comprises the amino acid sequence of SEQ ID NO: 51, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-C2orf73 fusion polypeptide of the disclosure comprises the amino acid sequence of SEQ ID NO: 52, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-AZI2 fusion polypeptide of the disclosure comprises the amino acid sequence of SEQ ID NO: 53, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MANBA fusion polypeptide of the disclosure comprises the amino acid sequence of SEQ ID NO: 54, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-CNTNAP5 fusion polypeptide of the disclosure comprises the amino acid sequence of SEQ ID NO: 55, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TANGO6 fusion polypeptide of the disclosure comprises the amino acid sequence of SEQ ID NO: 57, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-NFIA fusion polypeptide of the disclosure comprises the amino acid sequence of SEQ ID NO: 58, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-RPS6KA5 fusion polypeptide of the disclosure comprises the amino acid sequence of SEQ ID NO: 59, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TG fusion polypeptide of the disclosure comprises the amino acid sequence of SEQ ID NO: 60, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-LRRFIP2 fusion polypeptide of the disclosure comprises the amino acid sequence of SEQ ID NO: 61, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MYO5C fusion polypeptide of the disclosure comprises the amino acid sequence of SEQ ID NO: 62, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-AGAP1 fusion polypeptide of the disclosure comprises the amino acid sequence of SEQ ID NO: 63, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MED13L fusion polypeptide of the disclosure comprises the amino acid sequence of SEQ ID NO: 64, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MTBP fusion polypeptide of the disclosure comprises the amino acid sequence of SEQ ID NO: 65, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-SLC30A6 fusion polypeptide of the disclosure comprises the amino acid sequence of SEQ ID NO: 67, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-GMCL1 fusion polypeptide of the disclosure comprises the amino acid sequence of SEQ ID NO: 68, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-AGAP1 fusion polypeptide of the disclosure comprises the amino acid sequence of SEQ ID NO: 69, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-ZNF454 fusion polypeptide of the disclosure comprises the amino acid sequence of SEQ ID NO: 70, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-TTC28 fusion polypeptide of the disclosure comprises the amino acid sequence of SEQ ID NO: 71, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-NINJ2 fusion polypeptide of the disclosure comprises the amino acid sequence of SEQ ID NO: 72, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-UTRN fusion polypeptide of the disclosure comprises the amino acid sequence of SEQ ID NO: 73, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-ACTN4 fusion polypeptide of the disclosure comprises the amino acid sequence of SEQ ID NO: 74, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-CPSF7 fusion polypeptide of the disclosure comprises the amino acid sequence of SEQ ID NO: 75, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-SLC25A13 fusion polypeptide of the disclosure comprises the amino acid sequence of SEQ ID NO: 76, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-CTNND1 fusion polypeptide of the disclosure comprises the amino acid sequence of SEQ ID NO: 77, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-KLC4 fusion polypeptide of the disclosure comprises the amino acid sequence of SEQ ID NO: 78, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-BTBD9 fusion polypeptide of the disclosure comprises the amino acid sequence of SEQ ID NO: 79, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-CPQ fusion polypeptide of the disclosure comprises the amino acid sequence of SEQ ID NO: 81, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-KIFSC fusion polypeptide of the disclosure comprises the amino acid sequence of SEQ ID NO: 83, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-MAGOHB fusion polypeptide of the disclosure comprises the amino acid sequence of SEQ ID NO: 84, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto. In some embodiments, the ALK-COL3A1 fusion polypeptide of the disclosure comprises the amino acid sequence of SEQ ID NO: 86, or an amino acid sequence having at least about 70% (e.g., any of about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%) sequence homology thereto.

(iii) Cancers and Methods Related Thereto

Certain aspects of the present disclosure relate to methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; assessing a fusion nucleic acid molecule or polypeptide in a cancer in an individual; detecting the presence or absence of a cancer in an individual; monitoring progression or recurrence of a cancer in an individual; or identifying a candidate treatment for a cancer in an individual in need thereof.

In some embodiments of any of the methods provided herein, the methods comprise acquiring knowledge of or detecting in a sample from an individual having cancer, suspected of having cancer, being tested for cancer, or being treated for cancer, an ALK fusion nucleic acid molecule of the disclosure, e.g., an ALK fusion nucleic acid molecule described above, in Tables 1-7 and 9, and/or in the Examples herein, such as an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIFSC, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYOSC, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein. In other embodiments, the methods comprise acquiring knowledge of or detecting in a sample from an individual having cancer, suspected of having cancer, being tested for cancer, or being treated for cancer, an ALK fusion polypeptide of the disclosure, e.g., an ALK fusion polypeptide described above, and/or in Table 8, and/or in the Examples herein, such as an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion polypeptide described herein.

In some embodiments of any of the methods provided herein, detection of an ALK fusion nucleic acid molecule or polypeptide of the disclosure in a sample from an individual (e.g., an individual having cancer, suspected of having cancer, being tested for cancer, or being treated for cancer) identifies the individual as one who may benefit from a treatment comprising an anti-cancer therapy, such as an anti-cancer therapy provided herein, e.g., an ALK-targeted therapy.

In some embodiments, the methods comprise detecting, in a first sample obtained from the individual at a first time point, the presence or absence of an ALK fusion nucleic acid molecule or polypeptide of the disclosure. In some embodiments, the methods further comprise detecting, in a second sample obtained from the individual at a second time point after the first time point, the presence or absence of an ALK fusion nucleic acid molecule or polypeptide of the disclosure. In some embodiments, the methods further comprise providing an assessment of cancer progression or cancer recurrence in the individual based, at least in part, on the presence or absence of the ALK fusion nucleic acid molecule or polypeptide in the first sample and/or in the second sample. In some embodiments, the presence of the ALK fusion nucleic acid molecule or polypeptide in the first sample and/or in the second sample identifies the individual as having increased risk of cancer progression or cancer recurrence. In some embodiments, the methods further comprise selecting a treatment, administering a treatment, adjusting a treatment, adjusting the dose of a treatment, or applying a treatment to the individual based, at least in part, on detecting the presence of the ALK fusion nucleic acid molecule or polypeptide in the first sample and/or in the second sample, wherein the treatment comprises an anti-cancer therapy, such as an anti-cancer therapy provided herein, e.g., an ALK-targeted therapy.

In some embodiments, the methods comprise performing DNA sequencing on a sample obtained from an individual (e.g., an individual having cancer, suspected of having cancer, being tested for cancer, or being treated for cancer) to determine a sequencing mutation profile on a group of genes comprising one or more of ALK, ABCB11, ACTN4, AGAP1, APH1A, AZI2, BTBD9, C2orf73, CAPN14, CARMIL1, CASP8, CDC42BPA, CIB4, CNTNAP5, COL3A1, CPQ, CPSF7, CREBBP, CTBP1, CTNND1, CYP51A1, CYS1, EPHA2, FHOD3, FILIP1L, GMCL1, GPN1, GPR113, HADHA, HS1BP3, INTS9, ITGA6, KCTD18, KIF5C, KLC4, LINC00535, LRRFIP2, MAGOHB, MAMDC4, MANBA, MAP3K9, MED13L, METTL25, MTBP, MYH10, MYO5C, NFIA, NINJ2, OPRM1, OTX1, PAQR4, PDCD10, PDE3A, PELI1, PLEC, PTGER4, PTPRJ, QKI, RPS6KA5, SASH1, SEC16B, SKAP1, SLC25A13, SLC30A6, SNX17, SOX13, SRSF7, TANGO6, TG, TMCO3, TNS3, TRIM24, TTC28, UBE2L3, UBE3B, UTRN, VASP, WDR92, YPEL5, ZNF446, ZNF454, ZNF513, or ZSWIM2, or a gene listed in Table 1, or any combination thereof, wherein the sequencing mutation profile identifies the presence or absence of an ALK fusion nucleic acid molecule of the disclosure. In some embodiments, the methods further comprise identifying a candidate treatment for a cancer in an individual, based at least in part on the sequencing mutation profile. In some embodiments, the candidate treatment comprises an anti-cancer therapy, such as an anti-cancer therapy provided herein, e.g., an ALK-targeted therapy. In some embodiments, the sequencing mutation profile identifies the presence or absence of a fragment of the ALK fusion nucleic acid molecule, wherein the fragment comprises a breakpoint or fusion junction, e.g., one or more of the corresponding breakpoints described herein. In some embodiments, the presence of the ALK fusion nucleic acid molecule in the sample identifies the individual as one who may benefit from a treatment comprising an anti-cancer therapy, e.g., an anti-cancer therapy provided herein, such as an ALK-targeted therapy. In some embodiments, the presence of the ALK fusion nucleic acid molecule in the sample predicts the individual to have longer survival when treated with a treatment comprising an anti-cancer therapy, e.g., an ALK-targeted therapy, as compared to survival of an individual whose cancer does not comprise the ALK fusion nucleic acid molecule.

In some embodiments of any of the methods provided herein, the methods further comprise generating a report comprising one or more treatment options identified for the individual based at least in part on detection of the ALK fusion nucleic acid molecule or polypeptide in the sample, wherein the one or more treatment options comprise an anti-cancer therapy, such as an anti-cancer therapy provided herein, e.g., an ALK-targeted therapy.

In some embodiments of any of the methods provided herein, responsive to acquisition of knowledge of an ALK fusion nucleic acid molecule or polypeptide of the disclosure in a sample from an individual (e.g., an individual having cancer, suspected of having cancer, being tested for cancer, or being treated for cancer): (i) the individual is classified as a candidate to receive a treatment comprising an anti-cancer therapy, such as an anti-cancer therapy provided herein, e.g., an ALK-targeted therapy; and/or (ii) the individual is identified as likely to respond to a treatment that comprises an anti-cancer therapy, such as an anti-cancer therapy provided herein, e.g., an ALK-targeted therapy. In some embodiments, responsive to acquisition of knowledge of the ALK fusion nucleic acid molecule or polypeptide in a sample from the individual, the individual is predicted to have longer survival when treated with a treatment comprising an anti-cancer therapy, such as an anti-cancer therapy provided herein, e.g., an ALK-targeted therapy, as compared to survival of an individual whose cancer does not comprise or exhibit the ALK fusion nucleic acid molecule or polypeptide. In some embodiments, responsive to acquisition of knowledge of the ALK fusion nucleic acid molecule or polypeptide in a sample from the individual, the individual is predicted to have resistance to an anti-cancer therapy (e.g., a non-ALK-targeted therapy, and/or a prior anti-cancer therapy administered to the individual), the individual is predicted to respond to an anti-cancer therapy (e.g., an anti-cancer therapy provided herein, such as an ALK-targeted therapy), and/or the individual is predicted to have poor prognosis, e.g., when treated with a non-ALK-targeted therapy, as compared to an individual whose cancer does not comprise the fusion nucleic acid molecule or polypeptide.

In some embodiments, responsive to acquisition of knowledge of an ALK fusion nucleic acid molecule or polypeptide of the disclosure in a sample from an individual (e.g., an individual having cancer, suspected of having cancer, being tested for cancer, or being treated for cancer), the methods comprise administering to the individual an effective amount of a treatment that comprises an anti-cancer therapy, such as an anti-cancer therapy provided herein, e.g., an ALK-targeted therapy.

In some embodiments of any of the methods provided herein, the methods further comprise generating a report comprising one or more treatment options identified for the individual based, at least in part, on knowledge of an ALK fusion nucleic acid molecule or polypeptide of the disclosure in a sample from the individual, wherein the one or more treatment options comprise an anti-cancer therapy, such as an anti-cancer therapy provided herein, e.g., an ALK targeted therapy.

In some embodiments, acquiring knowledge of an ALK fusion nucleic acid molecule or polypeptide of the disclosure in a sample comprises detecting the ALK fusion nucleic acid molecule or polypeptide in the sample. In some embodiments of any of the methods provided herein, detecting an ALK fusion nucleic acid molecule of the disclosure comprises detecting a fragment of the fusion nucleic acid molecule comprising a breakpoint or fusion junction, e.g., one or more of the corresponding breakpoints described herein. In some embodiments of any of the methods provided herein, detecting an ALK fusion polypeptide of the disclosure comprises detecting a portion of the fusion polypeptide that is encoded by a fragment of an ALK fusion nucleic acid molecule that comprises a breakpoint or a fusion junction, e.g., one or more of the corresponding breakpoints described herein. In some embodiments of any of the methods provided herein, detecting an ALK fusion polypeptide of the disclosure comprises detecting a portion of the fusion polypeptide that comprises the fusion between the ALK polypeptide, or the portion thereof, and the polypeptide encoded by the other gene in the fusion, or the portion thereof.

In some embodiments, the methods of the disclosure further comprise providing an assessment of the ALK fusion nucleic acid molecule or polypeptide of the disclosure.

In some embodiments of any of the methods provided herein, the anti-cancer therapy in an ALK-targeted therapy. In some embodiments, the ALK-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an ALK fusion nucleic acid molecule or polypeptide, a treatment for ALK-positive or ALK-rearranged cancer, an ALK-targeted therapy being tested in a clinical trial, a treatment for cancer being tested in a clinical trial, a targeted therapy, a treatment being tested in a clinical trial for cancer comprising an ALK fusion nucleic acid molecule or polypeptide, or any combination thereof, e.g., a described in further detail below. In some embodiments, the ALK-targeted therapy is a kinase inhibitor, such as a kinase inhibitor described herein or known in the art. In some embodiments, the ALK-targeted therapy is a tyrosine kinase inhibitor described herein or known in the art. In some embodiments, the ALK-targeted therapy is a kinase inhibitor that inhibits the kinase activity of an ALK polypeptide. In some embodiments, the ALK-targeted therapy is a multi-kinase inhibitor or an ALK-specific inhibitor. In some embodiments, the ALK-targeted therapy comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707 (see, e.g., clinicaltrials.gov/ct2/show/NCT02695550), WX-0593 (see, e.g., clinicaltrials.gov/ct2/show/NCT04641754), alkotinib (see, e.g., clinicaltrials.gov/ct2/show/NCTO4211922), SIM1803-1A (see, e.g., clinicaltrials.gov/ct2/show/NCT04671849), PLB1003 (see, e.g., clinicaltrials.gov/ct2/show/NCT03130881), SAF-189s (see, e.g., clinicaltrials.gov/ct2/show/NCT04237805), PF03446962 (see, e.g., clinicaltrials.gov/ct2/show/NCT01620970), TQ-B3101 (see, e.g., clinicaltrials.gov/ct2/show/NCT04412564), APG-2449 (see, e.g., clinicaltrials.gov/ct2/show/NCT03917043), X-376 (see, e.g., Awad et al., Clinical advances in hematology & oncology: H&O vol. 12, 7 (2014):429-39), CEP-28122 (see, e.g., Cheng et al., Molecular cancer therapeutics vol. 11, 3 (2012): 670-9; and Spagnuolo et al., Expert opinion on emerging drugs vol. 23, 3 (2018): 231-241), and GSK1838705A (see, e.g., Sabbatini, Peter et al., Molecular cancer therapeutics vol. 8, 10 (2009): 2811-20). In some embodiments, the ALK-targeted therapy inhibits the expression of an ALK fusion nucleic acid molecule or polypeptide of the disclosure. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy. In some embodiments, the nucleic acid inhibits the expression of an ALK fusion nucleic acid molecule or polypeptide of the disclosure. In some embodiments, the nucleic acid comprises a double-stranded RNA (dsRNA), a small interfering RNA (siRNA), or a small hairpin RNA (shRNA), e.g., as described herein. In some embodiments, the ALK-targeted therapy is a first-line or front-line treatment for cancer.

In some embodiments of any of the methods provided herein, the methods further comprise acquiring knowledge of or detecting in a sample from an individual (e.g., an individual having cancer, suspected of having cancer, being tested for cancer, or being treated for cancer) a base substitution, a short insertion/deletion (indel), a copy number alteration, or a genomic rearrangement in one or more genes. In some embodiments, the one or more genes comprise one or more of ALK, ABCB11, ACTN4, AGAP1, APH1A, AZI2, BTBD9, C2orf73, CAPN14, CARMIL1, CASP8, CDC42BPA, CIB4, CNTNAP5, COL3A1, CPQ, CPSF7, CREBBP, CTBP1, CTNND1, CYP51A1, CYS1, EPHA2, FHOD3, FILIP1L, GMCL1, GPN1, GPR113, HADHA, HS1BP3, INTS9, ITGA6, KCTD18, KIF5C, KLC4, LINC00535, LRRFIP2, MAGOHB, MAMDC4, MANBA, MAP3K9, MED13L, METTL25, MTBP, MYH10, MYO5C, NFIA, NINJ2, OPRM1, OTX1, PAQR4, PDCD10, PDE3A, PELI1, PLEC, PTGER4, PTPRJ, QKI, RPS6KA5, SASH1, SEC16B, SKAP1, SLC25A13, SLC30A6, SNX17, SOX13, SRSF7, TANGO6, TG, TMCO3, TNS3, TRIM24, TTC28, UBE2L3, UBE3B, UTRN, VASP, WDR92, YPEL5, ZNF446, ZNF454, ZNF513, or ZSWIM2, or a gene listed in Table 1, or a panel of known/suspected oncogenes and/or tumor suppressors, or any combination thereof.

In some embodiments of any of the methods provided herein, the treatment or the one or more treatment options, e.g., the ALK-targeted therapy, further comprise an additional anti-cancer therapy, e.g., an ALK-targeted therapy in combination with an additional anti-cancer therapy. In some embodiments of any of the methods provided herein, the treatment or the one or more treatment options, e.g., the ALK-targeted therapy, further comprise administering an additional anti-cancer therapy to the individual, e.g., administering an ALK-targeted therapy in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof. In some embodiments, the additional anti-cancer therapy is an immunotherapy, such as an immune checkpoint inhibitor. In some embodiments, the additional anti-cancer therapy is a PD-1-, a CTLA4-, or a PD-L1-targeted agent. In some embodiments, the additional anti-cancer therapy is a heat shock protein 90 inhibitor (Golding et al., Molecular cancer vol. 17, 1 52, 2018; Pall, Current opinion in oncology vol. 27, 2 (2015):118-24), an EGFR inhibitor (Golding et al., Molecular cancer vol. 17, 1 52, 2018), a SHP2 inhibitor (Dardaei et al., Nature medicine vol. 24, 4 (2018): 512-517), a MEK inhibitor (Shrestha et al., Scientific reports vol. 9, 1 18842, 2019; Shrestha et al., The Journal of pharmacology and experimental therapeutics vol. 374, 1 (2020):134-140), an IGF-1R inhibitor (George, Journal of hematology & oncology vol. 12, 1 80, 2019), a vascular endothelial growth factor (VEGF)-targeted therapy (Makimoto et al., Acta medica Okayama vol. 74, 5 (2020): 371-379; Gristina et al., Pharmaceuticals (Basel, Switzerland) vol. 13, 12 474, 2020), an mTOR inhibitor (Kim et al., Anticancer research vol. 40, 3 (2020): 1395-1403), or any combination thereof.

In some embodiments, the individual has been previously treated, or is being treated, for cancer with a treatment for cancer, e.g., an anti-cancer therapy described herein or any other anti-cancer therapy or treatment known in the art. In some embodiments, the individual has been previously treated, or is being treated, for cancer with a kinase inhibitor. In some embodiments, an ALK fusion nucleic acid molecule and/or an ALK fusion polypeptide of the disclosure confer resistance of a cancer to a treatment for cancer, e.g., a prior treatment for cancer. In some embodiments, the cancer progressed on a prior treatment, such as a kinase inhibitor. In some embodiments, the individual has not been previously treated for cancer. In some embodiments, the individual, or the cancer, has not been previously treated with a kinase inhibitor. In some embodiments, the individual, or the cancer, is kinase inhibitor naïve.

In some embodiments of any of the methods provided herein, the cancer is a carcinoma, a sarcoma, a lymphoma, a leukemia, a myeloma, a germ cell cancer, or a blastoma. In some embodiments, the cancer is a solid tumor. In some embodiments, the cancer is a hematologic malignancy. In some embodiments, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments, the cancer is a non-small cell lung carcinoma, a leiomyosarcoma, a thyroid carcinoma, a colorectal cancer, a pancreatic cancer, or a malignant peritoneal mesothelioma. In some embodiments, the cancer is anus squamous cell carcinoma, brain glioblastoma (GBM), breast cancer (NOS), breast carcinoma (NOS), breast invasive ductal carcinoma (IDC), colon adenocarcinoma (CRC), esophagus adenocarcinoma, fallopian tube serous carcinoma, gallbladder adenocarcinoma, gallbladder carcinoma, gastroesophageal junction adenocarcinoma, lung adenocarcinoma, lung non-small cell lung carcinoma (NOS), lymph node Castleman's disease, lymph node lymphoma t-cell (NOS), ovary clear cell carcinoma, ovary endometrioid adenocarcinoma, ovary epithelial carcinoma (NOS), ovary high grade serous carcinoma, ovary serous carcinoma, pancreas cancer (NOS), pancreas ductal adenocarcinoma, pediatric bone osteosarcoma, pediatric skin melanoma, pediatric soft tissue sarcoma (NOS), pediatric soft tissue sarcoma undifferentiated, peritoneum serous carcinoma, prostate acinar adenocarcinoma, skin melanoma, small intestine adenocarcinoma, soft tissue leiomyosarcoma, soft tissue liposarcoma, soft tissue sarcoma (NOS), soft tissue sarcoma undifferentiated, thyroid papillary carcinoma, unknown primary adenocarcinoma, unknown primary carcinoma (NOS), unknown primary malignant neoplasm (NOS), unknown primary myoepithelial carcinoma, uterus carcinosarcoma, uterus endometrial adenocarcinoma endometrioid, uterus leiomyosarcoma, or vulva squamous cell carcinoma (SCC).

In some embodiments, the methods further comprise detecting the presence or absence of a cancer in a sample from the individual. In some embodiments, the methods further comprise administering an effective amount of anti-cancer therapy to the individual, e.g., an anti-cancer therapy described herein, such as an ALK-targeted therapy.

In some embodiments, any of the cancers described herein comprise any of the ALK fusion nucleic acid molecules of the disclosure, e.g., an ALK fusion nucleic acid molecule described above and/or in the Examples herein. In other embodiments, any of the cancers described herein comprise any of the ALK fusion polypeptides of the disclosure, e.g., an ALK fusion polypeptide described above and/or in the Examples herein. In some embodiments, the methods provided herein comprise acquiring knowledge of or detecting any of the ALK fusion nucleic acid molecules of the disclosure in a sample from an individual having any cancer known in the art, or any of the cancers described herein. In some embodiments, the methods provided herein comprise acquiring knowledge of or detecting any of the ALK fusion polypeptides of the disclosure in a sample from an individual having any cancer known in the art, or any of the cancers described herein. In some embodiments, the cancer is a cancer listed in Table 9, and the ALK fusion nucleic acid molecule is the corresponding ALK fusion nucleic acid molecule or polypeptide as listed in Table 9.

TABLE 9
Cancers and exemplary ALK fusions.

| ALK Fusion Nucleic Acid | |
|---|---|
| Molecule or Polypeptide | Cancer |
| ALK-COL3A1 | Soft tissue sarcoma (NOS) |
| ALK-CDC42BPA | Ovary serous carcinoma |
| ALK-EPHA2 | Colon adenocarcinoma (CRC) |
| ALK-MYO5C | Lung adenocarcinoma |
| ALK-TRIM24 | Lung adenocarcinoma |
| ALK-SKAP1 | Ovary endometrioid adenocarcinoma |
| ALK-UBE3B | Lung adenocarcinoma |
| ALK-TNS3 | Lymph node Castleman's disease |
| ALK-C2orf73 | Lung adenocarcinoma |
| ALK-AZI2 | Gastroesophageal junction adenocarcinoma |
| ALK-MANBA | Lung adenocarcinoma |
| ALK-CNTNAP5 | Lung adenocarcinoma |
| ALK-TANGO6 | Soft tissue sarcoma undifferentiated |
| ALK-NFIA | Soft tissue liposarcoma |
| ALK-RPS6KA5 | Peritoneum serous carcinoma |
| ALK-TG | Thyroid papillary carcinoma |
| ALK-LRRFIP2 | Lung non-small cell lung carcinoma (NOS) |
| ALK-MYO5C | Lung adenocarcinoma |
| ALK-AGAP1 | Unknown primary myoepithelial carcinoma |
| ALK-MED13L | PEDIATRIC Soft tissue sarcoma |
| | undifferentiated |
| ALK-MTBP | Ovary serous carcinoma |
| ALK-SLC30A6 | Lung non-small cell lung carcinoma (NOS) |
| ALK-GMCL1 | Prostate acinar adenocarcinoma |
| ALK-AGAP1 | Lung adenocarcinoma |
| ALK-ZNF454 | Lung non-small cell lung carcinoma (NOS) |
| ALK-TTC28 | Uterus carcinosarcoma |
| ALK-NINJ2 | Breast invasive ductal carcinoma (IDC) |
| ALK-UTRN | Pancreas ductal adenocarcinoma |
| ALK-ACTN4 | Lung adenocarcinoma |
| ALK-CPSF7 | Breast carcinoma (NOS) |
| ALK-SLC25A13 | Lung adenocarcinoma |
| ALK-CTNND1 | Lung adenocarcinoma |
| ALK-KLC4 | Lung adenocarcinoma |
| ALK-BTBD9 | Lung non-small cell lung carcinoma (NOS) |
| ALK-CPQ | Lung non-small cell lung carcinoma (NOS) |
| ALK-KIF5C | Unknown primary carcinoma (NOS) |
| ALK-MAGOHB | Anus squamous cell carcinoma |
| ALK-COL3A1 | Uterus leiomyosarcoma |
| ALK-OPRM1 | Lung adenocarcinoma |
| ALK-GPN1 | Skin melanoma |
| ALK-SEC16B | Esophagus adenocarcinoma |
| ALK-UBE2L3 | Skin melanoma |
| ALK-METTL25 | Lung adenocarcinoma |
| ALK-CYS1 | Lung adenocarcinoma |
| ALK-ABCB11 | Lung non-small cell lung carcinoma (NOS) |
| ALK-INTS9 | Lung adenocarcinoma |
| ALK-CIB4 | Lung adenocarcinoma |
| ALK-WDR92 | Soft tissue leiomyosarcoma |
| ALK-OTX1 | Lung adenocarcinoma |
| ALK-PDCD10 | Breast carcinoma (NOS) |
| ALK-PTGER4 | Prostate acinar adenocarcinoma |
| ALK-PTPRJ | Lung non-small cell lung carcinoma |
| | (NSCLC) (NOS) |
| ALK-ZSWIM2 | Lung adenocarcinoma |
| ALK-FHOD3 | Lung non-small cell lung carcinoma |
| | (NSCLC) (NOS) |
| ALK-FILIP1L | Pancreas ductal adenocarcinoma |
| ALK-ITGA6 | Pancreas cancer (NOS) |
| ALK-KCTD18 | Lung adenocarcinoma |
| ALK-MAMDC4 | Ovary clear cell carcinoma |
| ALK-PELI1 | Breast cancer (NOS) |
| ALK-LINC00535 | Lung non-small cell lung carcinoma (NOS) |
| ALK-CTBP1 | Lung adenocarcinoma |
| ALK-CARMIL1 | Lung adenocarcinoma |
| ALK-ZNF513 | Gallbladder adenocarcinoma |
| ALK-TMCO3 | Unknown primary adenocarcinoma |
| ALK-SRSF7 | Colon adenocarcinoma (CRC) |
| ALK-CASP8 | Vulva squamous cell carcinoma (SCC) |
| ALK-CYP51A1 | Ovary high grade serous carcinoma |
| ALK-GPR113 | Prostate acinar adenocarcinoma |
| ALK-HADHA | Fallopian tube serous carcinoma |
| ALK-LRRFIP2 | Lung adenocarcinoma |
| ALK-MYH10 | PEDIATRIC Soft tissue sarcoma (NOS) |
| ALK-PDE3A | PEDIATRIC Bone osteosarcoma |
| ALK-PLEC | Uterus endometrial adenocarcinoma |
| | endometrioid |
| ALK-QKI | Brain glioblastoma (GBM) |
| ALK-SASH1 | Brain glioblastoma (GBM) |
| ALK-SRSF7 | Pancreas ductal adenocarcinoma |
| ALK-VASP | Ovary clear cell carcinoma |
| ALK-ZNF446 | Ovary serous carcinoma |
| ALK-SOX13 | PEDIATRIC Skin melanoma |
| ALK-YPEL5 | Brain glioblastoma (GBM) |
| ALK-CAPN14 | Small intestine adenocarcinoma |
| ALK-MAP3K9 | Breast carcinoma (NOS) |
| ALK-SNX17 | Prostate acinar adenocarcinoma |
| ALK-HS1BP3 | Lung adenocarcinoma |
| ALK-CREBBP | Ovary serous carcinoma |
| ALK-PAQR4 | Breast carcinoma (NOS) |
| ALK-APH1A | Uterus leiomyosarcoma |

In some embodiments, the cancer is soft tissue sarcoma (NOS), and the ALK fusion nucleic acid molecule is any of the ALK-COL3A1 fusion nucleic acid molecules described herein or the ALK fusion polypeptide is any of the ALK-COL3A1 fusion polypeptides described herein. In some embodiments, the cancer is ovary serous carcinoma, and the ALK fusion nucleic acid molecule is any of the ALK-CDC42BPA fusion nucleic acid molecules described herein or the ALK fusion polypeptide is any of the ALK-CDC42BPA fusion polypeptides described herein. In some embodiments, the cancer is colon adenocarcinoma (CRC), and the ALK fusion nucleic acid molecule is any of the ALK-EPHA2 fusion nucleic acid molecules described herein or the ALK fusion polypeptide is any of the ALK-EPHA2 fusion polypeptides described herein. In some embodiments, the cancer is lung adenocarcinoma, and the ALK fusion nucleic acid molecule is any of the ALK-MYO5C fusion nucleic acid molecules described herein or the ALK fusion polypeptide is any of the ALK-MYO5C fusion polypeptides described herein. In some embodiments, the cancer is lung adenocarcinoma, and the ALK fusion nucleic acid molecule is any of the ALK-TRIM24 fusion nucleic acid molecules described herein or the ALK fusion polypeptide is any of the ALK-TRIM24 fusion polypeptides described herein. In some embodiments, the cancer is ovary endometrioid adenocarcinoma, and the ALK fusion nucleic acid molecule is any of the ALK-SKAP1 fusion nucleic acid molecules described herein or the ALK fusion polypeptide is any of the ALK-SKAP1 fusion polypeptides described herein. In some embodiments, the cancer is lung adenocarcinoma, and the ALK fusion nucleic acid molecule is any of the ALK-UBE3B fusion nucleic acid molecules described herein or the ALK fusion polypeptide is any of the ALK-UBE3B fusion polypeptides described herein. In some embodiments, the cancer is lymph node Castleman's disease, and the ALK fusion nucleic acid molecule is any of the ALK-TNS3 fusion nucleic acid molecules described herein or the ALK fusion polypeptide is any of the ALK-TNS3 fusion polypeptides described herein. In some embodiments, the cancer is lung adenocarcinoma, and the ALK fusion nucleic acid molecule is any of the ALK-C2orf73 fusion nucleic acid molecules described herein or the ALK fusion polypeptide is any of the ALK-C2orf73 fusion polypeptides described herein. In some embodiments, the cancer is gastroesophageal junction adenocarcinoma, and the ALK fusion nucleic acid molecule is any of the ALK-AZI2 fusion nucleic acid molecules described herein or the ALK fusion polypeptide is any of the ALK-AZI2 fusion polypeptides described herein. In some embodiments, the cancer is lung adenocarcinoma, and the ALK fusion nucleic acid molecule is any of the ALK-MANBA fusion nucleic acid molecules described herein or the ALK fusion polypeptide is any of the ALK-MANBA fusion polypeptides described herein. In some embodiments, the cancer is lung adenocarcinoma, and the ALK fusion nucleic acid molecule is any of the ALK-CNTNAP5 fusion nucleic acid molecules described herein or the ALK fusion polypeptide is any of the ALK-CNTNAP5 fusion polypeptides described herein. In some embodiments, the cancer is soft tissue sarcoma undifferentiated, and the ALK fusion nucleic acid molecule is any of the ALK-TANGO6 fusion nucleic acid molecules described herein or the ALK fusion polypeptide is any of the ALK-TANGO6 fusion polypeptides described herein. In some embodiments, the cancer is soft tissue liposarcoma, and the ALK fusion nucleic acid molecule is any of the ALK-NFIA fusion nucleic acid molecules described herein or the ALK fusion polypeptide is any of the ALK-NFIA fusion polypeptides described herein. In some embodiments, the cancer is peritoneum serous carcinoma, and the ALK fusion nucleic acid molecule is any of the ALK-RPS6KA5 fusion nucleic acid molecules described herein or the ALK fusion polypeptide is any of the ALK-RPS6KA5 fusion polypeptides described herein. In some embodiments, the cancer is thyroid papillary carcinoma, and the ALK fusion nucleic acid molecule is any of the ALK-TG fusion nucleic acid molecules described herein or the ALK fusion polypeptide is any of the ALK-TG fusion polypeptides described herein. In some embodiments, the cancer is lung non-small cell lung carcinoma (NOS), and the ALK fusion nucleic acid molecule is any of the ALK-LRRFIP2 fusion nucleic acid molecules described herein or the ALK fusion polypeptide is any of the ALK-LRRFIP2 fusion polypeptides described herein. In some embodiments, the cancer is lung adenocarcinoma, and the ALK fusion nucleic acid molecule is any of the ALK-MYO5C fusion nucleic acid molecules described herein or the ALK fusion polypeptide is any of the ALK-MYO5C fusion polypeptides described herein. In some embodiments, the cancer is unknown primary myoepithelial carcinoma, and the ALK fusion nucleic acid molecule is any of the ALK-AGAP1 fusion nucleic acid molecules described herein or the ALK fusion polypeptide is any of the ALK-AGAP1 fusion polypeptides described herein. In some embodiments, the cancer is pediatric soft tissue sarcoma undifferentiated, and the ALK fusion nucleic acid molecule is any of the ALK-MED13L fusion nucleic acid molecules described herein or the ALK fusion polypeptide is any of the ALK-MED13L fusion polypeptides described herein. In some embodiments, the cancer is ovary serous carcinoma, and the ALK fusion nucleic acid molecule is any of the ALK-MTBP fusion nucleic acid molecules described herein or the ALK fusion polypeptide is any of the ALK-MTBP fusion polypeptides described herein. In some embodiments, the cancer is lung non-small cell lung carcinoma (NOS), and the ALK fusion nucleic acid molecule is any of the ALK-SLC30A6 fusion nucleic acid molecules described herein or the ALK fusion polypeptide is any of the ALK-SLC30A6 fusion polypeptides described herein. In some embodiments, the cancer is prostate acinar adenocarcinoma, and the ALK fusion nucleic acid molecule is any of the ALK-GMCL1 fusion nucleic acid molecules described herein or the ALK fusion polypeptide is any of the ALK-GMCL1 fusion polypeptides described herein. In some embodiments, the cancer is lung adenocarcinoma, and the ALK fusion nucleic acid molecule is any of the ALK-AGAP1 fusion nucleic acid molecules described herein or the ALK fusion polypeptide is any of the ALK-AGAP1 fusion polypeptides described herein. In some embodiments, the cancer is lung non-small cell lung carcinoma (NOS), and the ALK fusion nucleic acid molecule is any of the ALK-ZNF454 fusion nucleic acid molecules described herein or the ALK fusion polypeptide is any of the ALK-ZNF454 fusion polypeptides described herein. In some embodiments, the cancer is uterus carcinosarcoma, and the ALK fusion nucleic acid molecule is any of the ALK-TTC28 fusion nucleic acid molecules described herein or the ALK fusion polypeptide is any of the ALK-TTC28 fusion polypeptides described herein. In some embodiments, the cancer is breast invasive ductal carcinoma (IDC), and the ALK fusion nucleic acid molecule is any of the ALK-NINJ2 fusion nucleic acid molecules described herein or the ALK fusion polypeptide is any of the ALK-NINJ2 fusion polypeptides described herein. In some embodiments, the cancer is pancreas ductal adenocarcinoma, and the ALK fusion nucleic acid molecule is any of the ALK-UTRN fusion nucleic acid molecules described herein or the ALK fusion polypeptide is any of the ALK-UTRN fusion polypeptides described herein. In some embodiments, the cancer is lung adenocarcinoma, and the ALK fusion nucleic acid molecule is any of the ALK-ACTN4 fusion nucleic acid molecules described herein or the ALK fusion polypeptide is any of the ALK-ACTN4 fusion polypeptides described herein. In some embodiments, the cancer is breast carcinoma (NOS), and the ALK fusion nucleic acid molecule is any of the ALK-CPSF7 fusion nucleic acid molecules described herein or the ALK fusion polypeptide is any of the ALK-CPSF7 fusion polypeptides described herein. In some embodiments, the cancer is lung adenocarcinoma, and the ALK fusion nucleic acid molecule is any of the ALK-SLC25A13 fusion nucleic acid molecules described herein or the ALK fusion polypeptide is any of the ALK-SLC25A13 fusion polypeptides described herein. In some embodiments, the cancer is lung adenocarcinoma, and the ALK fusion nucleic acid molecule is any of the ALK-CTNND1 fusion nucleic acid molecules described herein or the ALK fusion polypeptide is any of the ALK-CTNND1 fusion polypeptides described herein. In some embodiments, the cancer is lung adenocarcinoma, and the ALK fusion nucleic acid molecule is any of the ALK-KLC4 fusion nucleic acid molecules described herein or the ALK fusion polypeptide is any of the ALK-KLC4 fusion polypeptides described herein. In some embodiments, the cancer is lung non-small cell lung carcinoma (NOS), and the ALK fusion nucleic acid molecule is any of the ALK-BTBD9 fusion nucleic acid molecules described herein or the ALK fusion polypeptide is any of the ALK-BTBD9 fusion polypeptides described herein. In some embodiments, the cancer is lung non-small cell lung carcinoma (NOS), and the ALK fusion nucleic acid molecule is any of the ALK-CPQ fusion nucleic acid molecules described herein or the ALK fusion polypeptide is any of the ALK-CPQ fusion polypeptides described herein. In some embodiments, the cancer is unknown primary carcinoma (NOS), and the ALK fusion nucleic acid molecule is any of the ALK-KIF5C fusion nucleic acid molecules described herein or the ALK fusion polypeptide is any of the ALK-KIF5C fusion polypeptides described herein. In some embodiments, the cancer is anus squamous cell carcinoma, and the ALK fusion nucleic acid molecule is any of the ALK-MAGOHB fusion nucleic acid molecules described herein or the ALK fusion polypeptide is any of the ALK-MAGOHB fusion polypeptides described herein. In some embodiments, the cancer is uterus leiomyosarcoma, and the ALK fusion nucleic acid molecule is any of the ALK-COL3A1 fusion nucleic acid molecules described herein or the ALK fusion polypeptide is any of the ALK-COL3A1 fusion polypeptides described herein. In some embodiments, the cancer is lung adenocarcinoma, and the ALK fusion nucleic acid molecule is any of the ALK-OPRM1 fusion nucleic acid molecules described herein or the ALK fusion polypeptide is any of the ALK-OPRM1 fusion polypeptides described herein. In some embodiments, the cancer is skin melanoma, and the ALK fusion nucleic acid molecule is any of the ALK-GPN1 fusion nucleic acid molecules described herein or the ALK fusion polypeptide is any of the ALK-GPN1 fusion polypeptides described herein. In some embodiments, the cancer is esophagus adenocarcinoma, and the ALK fusion nucleic acid molecule is any of the ALK-SEC16B fusion nucleic acid molecules described herein or the ALK fusion polypeptide is any of the ALK-SEC16B fusion polypeptides described herein. In some embodiments, the cancer is skin melanoma, and the ALK fusion nucleic acid molecule is any of the ALK-UBE2L3 fusion nucleic acid molecules described herein or the ALK fusion polypeptide is any of the ALK-UBE2L3 fusion polypeptides described herein. In some embodiments, the cancer is lung adenocarcinoma, and the ALK fusion nucleic acid molecule is any of the ALK-METTL25 fusion nucleic acid molecules described herein or the ALK fusion polypeptide is any of the ALK-METTL25 fusion polypeptides described herein. In some embodiments, the cancer is lung adenocarcinoma, and the ALK fusion nucleic acid molecule is any of the ALK-CYS1 fusion nucleic acid molecules described herein or the ALK fusion polypeptide is any of the ALK-CYS1 fusion polypeptides described herein. In some embodiments, the cancer is lung non-small cell lung carcinoma (NOS), and the ALK fusion nucleic acid molecule is any of the ALK-ABCB11 fusion nucleic acid molecules described herein or the ALK fusion polypeptide is any of the ALK-ABCB11 fusion polypeptides described herein. In some embodiments, the cancer is lung adenocarcinoma, and the ALK fusion nucleic acid molecule is any of the ALK-INTS9 fusion nucleic acid molecules described herein or the ALK fusion polypeptide is any of the ALK-INTS9 fusion polypeptides described herein. In some embodiments, the cancer is lung adenocarcinoma, and the ALK fusion nucleic acid molecule is any of the ALK-CIB4 fusion nucleic acid molecules described herein or the ALK fusion polypeptide is any of the ALK-CIB4 fusion polypeptides described herein. In some embodiments, the cancer is soft tissue leiomyosarcoma, and the ALK fusion nucleic acid molecule is any of the ALK-WDR92 fusion nucleic acid molecules described herein or the ALK fusion polypeptide is any of the ALK-WDR92 fusion polypeptides described herein. In some embodiments, the cancer is lung adenocarcinoma, and the ALK fusion nucleic acid molecule is any of the ALK-OTX1 fusion nucleic acid molecules described herein or the ALK fusion polypeptide is any of the ALK-OTX1 fusion polypeptides described herein. In some embodiments, the cancer is breast carcinoma (NOS), and the ALK fusion nucleic acid molecule is any of the ALK-PDCD10 fusion nucleic acid molecules described herein or the ALK fusion polypeptide is any of the ALK-PDCD10 fusion polypeptides described herein. In some embodiments, the cancer is prostate acinar adenocarcinoma, and the ALK fusion nucleic acid molecule is any of the ALK-PTGER4 fusion nucleic acid molecules described herein or the ALK fusion polypeptide is any of the ALK-PTGER4 fusion polypeptides described herein. In some embodiments, the cancer is lung non-small cell lung carcinoma (NSCLC) (NOS), and the ALK fusion nucleic acid molecule is any of the ALK-PTPRJ fusion nucleic acid molecules described herein or the ALK fusion polypeptide is any of the ALK-PTPRJ fusion polypeptides described herein. In some embodiments, the cancer is lung adenocarcinoma, and the ALK fusion nucleic acid molecule is any of the ALK-ZSWIM2 fusion nucleic acid molecules described herein or the ALK fusion polypeptide is any of the ALK-ZSWIM2 fusion polypeptides described herein. In some embodiments, the cancer is lung non-small cell lung carcinoma (NSCLC) (NOS), and the ALK fusion nucleic acid molecule is any of the ALK-FHOD3 fusion nucleic acid molecules described herein or the ALK fusion polypeptide is any of the ALK-FHOD3 fusion polypeptides described herein. In some embodiments, the cancer is pancreas ductal adenocarcinoma, and the ALK fusion nucleic acid molecule is any of the ALK-FILIP1L fusion nucleic acid molecules described herein or the ALK fusion polypeptide is any of the ALK-FILIP1L fusion polypeptides described herein. In some embodiments, the cancer is pancreas cancer (NOS), and the ALK fusion nucleic acid molecule is any of the ALK-ITGA6 fusion nucleic acid molecules described herein or the ALK fusion polypeptide is any of the ALK-ITGA6 fusion polypeptides described herein. In some embodiments, the cancer is lung adenocarcinoma, and the ALK fusion nucleic acid molecule is any of the ALK-KCTD18 fusion nucleic acid molecules described herein or the ALK fusion polypeptide is any of the ALK-KCTD18 fusion polypeptides described herein. In some embodiments, the cancer is ovary clear cell carcinoma, and the ALK fusion nucleic acid molecule is any of the ALK-MAMDC4 fusion nucleic acid molecules described herein or the ALK fusion polypeptide is any of the ALK-MAMDC4 fusion polypeptides described herein. In some embodiments, the cancer is breast cancer (NOS), and the ALK fusion nucleic acid molecule is any of the ALK-PELI1 fusion nucleic acid molecules described herein or the ALK fusion polypeptide is any of the ALK-PELI1 fusion polypeptides described herein. In some embodiments, the cancer is lung non-small cell lung carcinoma (NOS), and the ALK fusion nucleic acid molecule is any of the ALK-LINC00535 fusion nucleic acid molecules described herein or the ALK fusion polypeptide is any of the ALK-LINC00535 fusion polypeptides described herein. In some embodiments, the cancer is lung adenocarcinoma, and the ALK fusion nucleic acid molecule is any of the ALK-CTBP1 fusion nucleic acid molecules described herein or the ALK fusion polypeptide is any of the ALK-CTBP1 fusion polypeptides described herein. In some embodiments, the cancer is lung adenocarcinoma, and the ALK fusion nucleic acid molecule is any of the ALK-CARMIL1 fusion nucleic acid molecules described herein or the ALK fusion polypeptide is any of the ALK-CARMIL1 fusion polypeptides described herein. In some embodiments, the cancer is gallbladder adenocarcinoma, and the ALK fusion nucleic acid molecule is any of the ALK-ZNF513 fusion nucleic acid molecules described herein or the ALK fusion polypeptide is any of the ALK-ZNF513 fusion polypeptides described herein. In some embodiments, the cancer is unknown primary adenocarcinoma, and the ALK fusion nucleic acid molecule is any of the ALK-TMCO3 fusion nucleic acid molecules described herein or the ALK fusion polypeptide is any of the ALK-TMCO3 fusion polypeptides described herein. In some embodiments, the cancer is colon adenocarcinoma (CRC), and the ALK fusion nucleic acid molecule is any of the ALK-SRSF7 fusion nucleic acid molecules described herein or the ALK fusion polypeptide is any of the ALK-SRSF7 fusion polypeptides described herein. In some embodiments, the cancer is vulva squamous cell carcinoma (SCC), and the ALK fusion nucleic acid molecule is any of the ALK-CASP8 fusion nucleic acid molecules described herein or the ALK fusion polypeptide is any of the ALK-CASP8 fusion polypeptides described herein. In some embodiments, the cancer is ovary high grade serous carcinoma, and the ALK fusion nucleic acid molecule is any of the ALK-CYP51A1 fusion nucleic acid molecules described herein or the ALK fusion polypeptide is any of the ALK-CYP51A1 fusion polypeptides described herein. In some embodiments, the cancer is prostate acinar adenocarcinoma, and the ALK fusion nucleic acid molecule is any of the ALK-GPR113 fusion nucleic acid molecules described herein or the ALK fusion polypeptide is any of the ALK-GPR113 fusion polypeptides described herein. In some embodiments, the cancer is fallopian tube serous carcinoma, and the ALK fusion nucleic acid molecule is any of the ALK-HADHA fusion nucleic acid molecules described herein or the ALK fusion polypeptide is any of the ALK-HADHA fusion polypeptides described herein. In some embodiments, the cancer is lung adenocarcinoma, and the ALK fusion nucleic acid molecule is any of the ALK-LRRFIP2 fusion nucleic acid molecules described herein or the ALK fusion polypeptide is any of the ALK-LRRFIP2 fusion polypeptides described herein. In some embodiments, the cancer is pediatric soft tissue sarcoma (NOS), and the ALK fusion nucleic acid molecule is any of the ALK-MYH10 fusion nucleic acid molecules described herein or the ALK fusion polypeptide is any of the ALK-MYH10 fusion polypeptides described herein. In some embodiments, the cancer is pediatric bone osteosarcoma, and the ALK fusion nucleic acid molecule is any of the ALK-PDE3A fusion nucleic acid molecules described herein or the ALK fusion polypeptide is any of the ALK-PDE3A fusion polypeptides described herein. In some embodiments, the cancer is uterus endometrial adenocarcinoma endometrioid, and the ALK fusion nucleic acid molecule is any of the ALK-PLEC fusion nucleic acid molecules described herein or the ALK fusion polypeptide is any of the ALK-PLEC fusion polypeptides described herein. In some embodiments, the cancer is brain glioblastoma (GBM), and the ALK fusion nucleic acid molecule is any of the ALK-QKI fusion nucleic acid molecules described herein or the ALK fusion polypeptide is any of the ALK-QKI fusion polypeptides described herein. In some embodiments, the cancer is brain glioblastoma (GBM), and the ALK fusion nucleic acid molecule is any of the ALK-SASH1 fusion nucleic acid molecules described herein or the ALK fusion polypeptide is any of the ALK-SASH1 fusion polypeptides described herein. In some embodiments, the cancer is pancreas ductal adenocarcinoma, and the ALK fusion nucleic acid molecule is any of the ALK-SRSF7 fusion nucleic acid molecules described herein or the ALK fusion polypeptide is any of the ALK-SRSF7 fusion polypeptides described herein. In some embodiments, the cancer is ovary clear cell carcinoma, and the ALK fusion nucleic acid molecule is any of the ALK-VASP fusion nucleic acid molecules described herein or the ALK fusion polypeptide is any of the ALK-VASP fusion polypeptides described herein. In some embodiments, the cancer is ovary serous carcinoma, and the ALK fusion nucleic acid molecule is any of the ALK-ZNF446 fusion nucleic acid molecules described herein or the ALK fusion polypeptide is any of the ALK-ZNF446 fusion polypeptides described herein. In some embodiments, the cancer is pediatric skin melanoma, and the ALK fusion nucleic acid molecule is any of the ALK-SOX13 fusion nucleic acid molecules described herein or the ALK fusion polypeptide is any of the ALK-SOX13 fusion polypeptides described herein. In some embodiments, the cancer is brain glioblastoma (GBM), and the ALK fusion nucleic acid molecule is any of the ALK-YPEL5 fusion nucleic acid molecules described herein or the ALK fusion polypeptide is any of the ALK-YPEL5 fusion polypeptides described herein. In some embodiments, the cancer is small intestine adenocarcinoma, and the ALK fusion nucleic acid molecule is any of the ALK-CAPN14 fusion nucleic acid molecules described herein or the ALK fusion polypeptide is any of the ALK-CAPN14 fusion polypeptides described herein. In some embodiments, the cancer is breast carcinoma (NOS), and the ALK fusion nucleic acid molecule is any of the ALK-MAP3K9 fusion nucleic acid molecules described herein or the ALK fusion polypeptide is any of the ALK-MAP3K9 fusion polypeptides described herein. In some embodiments, the cancer is prostate acinar adenocarcinoma, and the ALK fusion nucleic acid molecule is any of the ALK-SNX17 fusion nucleic acid molecules described herein or the ALK fusion polypeptide is any of the ALK-SNX17 fusion polypeptides described herein. In some embodiments, the cancer is lung adenocarcinoma, and the ALK fusion nucleic acid molecule is any of the ALK-HS1BP3 fusion nucleic acid molecules described herein or the ALK fusion polypeptide is any of the ALK-HS1BP3 fusion polypeptides described herein. In some embodiments, the cancer is ovary serous carcinoma, and the ALK fusion nucleic acid molecule is any of the ALK-CREBBP fusion nucleic acid molecules described herein or the ALK fusion polypeptide is any of the ALK-CREBBP fusion polypeptides described herein. In some embodiments, the cancer is breast carcinoma (NOS), and the ALK fusion nucleic acid molecule is any of the ALK-PAQR4 fusion nucleic acid molecules described herein or the ALK fusion polypeptide is any of the ALK-PAQR4 fusion polypeptides described herein. In some embodiments, the cancer is uterus leiomyosarcoma, and the ALK fusion nucleic acid molecule is any of the ALK-APH1A fusion nucleic acid molecules described herein or the ALK fusion polypeptide is any of the ALK-APH1A fusion polypeptides described herein.

In some embodiments of any of the methods provided herein, the sample is a sample described below. In some embodiments, the sample is obtained from the individual or from the cancer. In some embodiments, the methods further comprise obtaining the sample, e.g., from the individual or from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof. In some embodiments, the fusion nucleic acid molecule or polypeptide is detected in a tissue biopsy sample, in a liquid biopsy sample, or in both a tissue biopsy sample and a liquid biopsy sample, from the individual.

### B. Detection of ALK Fusion Nucleic Acid Molecules and Polypeptides

Certain aspects of the present disclosure relate to detection of an ALK fusion nucleic acid molecule of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein) e.g., in a patient sample. In some embodiments, the fusion nucleic acid molecule is detected in vitro.

Other aspects of the present disclosure relate to detection of an ALK fusion polypeptide of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein) e.g., in a patient sample. In some embodiments, the fusion polypeptide is detected in vitro.

### (i) Detection of ALK Fusion Nucleic Acid Molecules

Methods for detecting an ALK fusion nucleic acid molecule of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein), are known in the art. For example, in some embodiments, an ALK fusion nucleic acid molecule is detected by sequencing part or all of a gene involved in the fusion nucleic acid molecule, e.g., an ALK gene, and/or a corresponding fusion partner gene described herein (e.g., as described herein, for example in any of Tables 1-7, and/or in the Examples herein), by next-generation or other sequencing of DNA, RNA, or cDNA. In some embodiments, an ALK fusion nucleic acid molecule of the disclosure is detected by PCR amplification of DNA, RNA, or cDNA. In some embodiments, an ALK fusion nucleic acid molecule of the disclosure is detected by in situ hybridization using one or more polynucleotides that hybridize to a locus involved in the fusion nucleic acid molecule, e.g., an ALK locus, and/or a corresponding fusion partner gene locus described herein (e.g., in Tables 1-7, and/or in the Examples herein), e.g., using fluorescence in situ hybridization (FISH). In some embodiments, an ALK fusion nucleic acid molecule of the disclosure is detected in a cancer or tumor cell, e.g., using tumor tissue, such as from a tumor biopsy or other tumor specimen; in a circulating cancer or tumor cell, e.g., using a liquid biopsy, such as from blood, plasma, cerebrospinal fluid, sputum, stool, urine, or saliva; or in circulating tumor DNA (ctDNA), e.g., using a liquid biopsy, such as from blood, plasma, cerebrospinal fluid, sputum, stool, urine, or saliva.

Exemplary and non-limiting methods for detecting an ALK fusion nucleic acid molecule of the disclosure are provided below.

In some embodiments, an ALK fusion nucleic acid molecule of the disclosure is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a nucleic acid molecule, are described in U.S. Pat. No. 9,340,830 and in WO2012092426A1, which are hereby incorporated by reference in their entirety. In some embodiments, an ALK fusion nucleic acid molecule of the disclosure is detected by sequencing. In some embodiments, the sequencing comprises a massively parallel sequencing (MPS) technique, whole genome sequencing (WGS), whole exome sequencing, targeted sequencing, direct sequencing, or a Sanger sequencing technique. In some embodiments, the massively parallel sequencing (MPS) technique comprises next-generation sequencing (NGS).

In some embodiments, an ALK fusion nucleic acid molecule of the disclosure is detected using an in situ hybridization method, such as a fluorescence in situ hybridization (FISH) method.

In some embodiments, FISH analysis is used to identify the chromosomal rearrangement resulting in an ALK fusion nucleic acid molecule as described herein. In some embodiments, FISH analysis is used to identify an RNA molecule comprising or encoding an ALK fusion nucleic acid molecule of the disclosure. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion, e.g., at one or more exons and or introns of the gene, are utilized. In normal cells (i.e., cells not having a fusion nucleic acid molecule described herein), both probes are observed (or a secondary color is observed due to the close proximity of the two genes of the gene fusion); and in cells having a fusion nucleic acid molecule described herein, only a single gene probe is observed due to the presence of a rearrangement resulting in the fusion nucleic acid molecule.

In some embodiments, an ALK fusion nucleic acid molecule of the disclosure is detected using an array-based method, such as array-based comparative genomic hybridization (CGH) methods. In array-based CGH methods, a first sample of nucleic acids (e.g., from a sample, such as from a tumor, or a tissue or liquid biopsy) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor, or a tissue or liquid biopsy) is labeled and hybridized to a second array (with identical content) and absolute signals are read. Copy number differences are calculated based on absolute signals from the two arrays.

In some embodiments, an ALK fusion nucleic acid molecule of the disclosure is detected using an amplification-based method. As is known in the art, in such amplification-based methods, a sample of nucleic acids, such as a sample obtained from an individual, a tumor or a tissue or liquid biopsy, is used as a template in an amplification reaction (e.g., Polymerase Chain Reaction (PCR)) using one or more oligonucleotides or primers, e.g., such as one or more oligonucleotides or primers provided herein. The presence of an ALK fusion nucleic acid molecule of the disclosure in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

In some embodiments, an ALK fusion nucleic acid molecule of the disclosure is detected using a sequencing method. Any method of sequencing known in the art can be used to detect an ALK fusion nucleic acid molecule provided herein. Exemplary sequencing methods that may be used to detect an ALK fusion nucleic acid molecule provided herein include those based on techniques developed by Maxam and Gilbert or Sanger. Automated sequencing procedures may also be used, e.g., including sequencing by mass spectrometry.

In some embodiments, an ALK fusion nucleic acid molecule of the disclosure is detected using hybrid capture-based sequencing (hybrid capture-based NGS), e.g., using adaptor ligation-based libraries. See, e.g., Frampton, G. M. et al. (2013) Nat. Biotech. 31:1023-1031, which is hereby incorporated by reference. In some embodiments, an ALK fusion nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 10 molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect an ALK fusion nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect an ALK fusion nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

In some embodiments of any of the methods provided herein, the methods may comprise one or more of the steps of: (i) obtaining a sample from an individual (e.g., an individual having, suspected of having, or determined to have cancer), (ii) extracting nucleic acid molecules (e.g., a mixture of tumor or cancer nucleic acid molecules and non-tumor or non-cancer nucleic acid molecules) from the sample, (iii) ligating one or more adapters to the nucleic acid molecules extracted from the sample (e.g., one or more amplification primers, flow cell adapter sequences, substrate adapter sequences, sample index sequences, or unique molecular identifier (UMI) sequences), (iv) amplifying the nucleic acid molecules (e.g., using a polymerase chain reaction (PCR) amplification technique, a non-PCR amplification technique, or an isothermal amplification technique), (v) capturing nucleic acid molecules from the amplified nucleic acid molecules (e.g., by hybridization to one or more bait molecules, where the bait molecules each comprise one or more nucleic acid molecules (e.g., capture nucleic acid molecules) that each comprise a region that is complementary to a region of a captured nucleic acid molecule), (vi) sequencing the nucleic acid molecules extracted from the sample (or library proxies derived therefrom) using, e.g., a next-generation (massively parallel) sequencing technique, a whole genome sequencing (WGS) technique, a whole exome sequencing technique, a targeted sequencing technique, a direct sequencing technique, or a Sanger sequencing technique) using, e.g., a next-generation (massively parallel) sequencer, and (vii) generating, displaying, transmitting, and/or delivering a report (e.g., an electronic, web-based, or paper report) to the individual (or patient), a caregiver, a healthcare provider, a physician, an oncologist, an electronic medical record system, a hospital, a clinic, a third-party payer, an insurance company, or a government office. In some instances, the report comprises output from the methods described herein. In some instances, all or a portion of the report may be displayed in a graphical user interface of an online or web-based healthcare portal. In some instances, the report is transmitted via a computer network or peer-to-peer connection.

In some embodiments of any of the methods provided herein, the methods may comprise one or more of the steps of: (a) providing a plurality of nucleic acid molecules obtained from a sample from an individual (e.g., an individual having, suspected of having or determined to have cancer), wherein the plurality of nucleic acid molecules comprises nucleic acid molecules corresponding to an ALK fusion nucleic acid molecule of the disclosure; (b) ligating one or more adapters onto one or more nucleic acid molecules from the plurality of nucleic acid molecules; (c) amplifying the one or more ligated nucleic acid molecules from the plurality of nucleic acid molecules; (d) capturing amplified nucleic acid molecules from the amplified nucleic acid molecules; (e) sequencing, by a sequencer, the captured nucleic acid molecules to obtain a plurality of sequence reads that represent the captured nucleic acid molecules, wherein one or more of the plurality of sequence reads correspond to the ALK fusion nucleic acid molecule; (f) analyzing the plurality of sequence reads; and (g) based on the analysis, detecting the presence or absence of the ALK fusion nucleic acid molecule in the sample. In some embodiments, the methods further comprise receiving, at one or more processors, sequence read data for the plurality of sequence reads. In some embodiments, the analyzing the plurality of sequence reads comprises identifying, using the one or more processors, the presence or absence of sequence reads corresponding to the fusion nucleic acid molecule. In some embodiments, the amplified nucleic acid molecules are captured by hybridization with one or more bait molecules.

In some embodiments of any of the methods provided herein, the methods may comprise one or more of the steps of: (a) providing a sample from an individual (e.g., an individual having, suspected of having or determined to have cancer), wherein the sample comprises a plurality of nucleic acid molecules; (b) preparing a nucleic acid sequencing library from the plurality of nucleic acid molecules in the sample; (c) amplifying said library; (d) selectively enriching for one or more nucleic acid molecules comprising nucleotide sequences corresponding to an ALK fusion nucleic acid molecule of the disclosure in said library to produce an enriched sample; (e) sequencing the enriched sample, thereby producing a plurality of sequence reads; (f) analyzing the plurality of sequence reads for the presence of the ALK fusion nucleic acid molecule; (g) detecting, based on the analyzing step, the presence or absence of the ALK fusion nucleic acid molecule in the sample from the individual.

In some embodiments of any of the methods provided herein, the plurality of nucleic acid molecules comprises a mixture of cancer nucleic acid molecules and non-cancer nucleic acid molecules. In some embodiments, the cancer nucleic acid molecules are derived from a tumor portion of a heterogeneous tissue biopsy sample, and the non-cancer nucleic acid molecules are derived from a normal portion of the heterogeneous tissue biopsy sample. In some embodiments, the sample comprises a liquid biopsy sample, and the cancer nucleic acid molecules are derived from a circulating tumor DNA (ctDNA) fraction of the liquid biopsy sample; and the non-cancer nucleic acid molecules are derived from a non-tumor fraction of the liquid biopsy sample or a cell-free DNA (cfDNA) fraction of the liquid biopsy sample.

In some embodiments of any of the methods, the one or more adapters comprise amplification primers, flow cell adaptor sequences, substrate adapter sequences, sample index sequences, or unique molecular identifier (UMI) sequences. In some embodiments, the one or more adapters comprise one or more sample index sequences. As is known in the art, sample indexes allow the sequencing of multiple samples on the same instrument flow cell or chip (i.e., multiplexing). Sample indexes are typically between about 8 and about 10 bases in length, and comprise a nucleotide sequence specific to a sample that is used to assign sequence reads to the correct sample during data analysis. In some embodiments, the one or more adapters comprise one or more unique molecule identifiers (UMIs). As is known in the art, UMIs comprise short nucleotide sequences that include a unique barcode that is incorporated into each molecule in a given sample library. UMIs are useful for identifying PCR duplicates created during library amplification steps, and/or for reducing the rate of false-positive variant calls and increasing variant detection, since variant alleles present in the original sample (true variants) can be distinguished from errors introduced during library preparation, target enrichment, or sequencing.

In some embodiments, the selectively enriching comprises: (a) combining one or more bait molecules with the library, thereby hybridizing the one or more bait molecules to one or more nucleic acid molecules comprising nucleotide sequences corresponding to the ALK fusion nucleic acid molecule and producing nucleic acid hybrids; and (b) isolating the nucleic acid hybrids to produce the enriched sample. In some embodiments, the captured nucleic acid molecules are captured from the amplified nucleic acid molecules by hybridization to one or more bait molecules. In some embodiments, the amplifying comprises performing a polymerase chain reaction (PCR) amplification technique, a non-PCR amplification technique, or an isothermal amplification technique. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the sequencing comprises a massively parallel sequencing technique, and the massively parallel sequencing technique comprises next generation sequencing (NGS). In some embodiments, the sequencer comprises a next generation sequencer.

In some embodiments of any of the methods provided herein, the methods further comprise selectively enriching for one or more nucleic acids in the sample comprising nucleotide sequences corresponding to the ALK fusion nucleic acid molecule of the disclosure. In some embodiments, the selectively enriching produces an enriched sample. In some embodiments, the selectively enriching comprises: (a) combining one or more bait molecules with the sample, thereby hybridizing the one or more bait molecules to one or more nucleic acids in the sample comprising nucleotide sequences corresponding to the ALK fusion nucleic acid molecule and producing nucleic acid hybrids; and (b) isolating the nucleic acid hybrids to produce the enriched sample. In some embodiments, the selectively enriching comprises amplifying the one or more nucleic acids comprising nucleotide sequences corresponding to the ALK fusion nucleic acid molecule using a polymerase chain reaction (PCR) to produce an enriched sample. In some embodiments, the methods further comprise sequencing the enriched sample.

In some embodiments of any of the methods provided herein, the methods further comprise generating a molecular profile for the individual or the sample, based, at least in part, on detecting the presence or absence of the ALK fusion nucleic acid molecule. In some embodiments, the molecular profile for the individual or sample further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test.

In some embodiments of any of the methods provided herein, the methods further comprise selecting a treatment, administering a treatment, or applying a treatment to the individual based on the generated molecular profile, wherein the treatment comprises an anti-cancer therapy, e.g., as described herein, e.g., an ALK-targeted therapy.

In some embodiments of any of the methods provided herein, the methods further comprise generating a report indicating the presence or absence of the ALK fusion nucleic acid molecule in the sample. In some embodiments, the methods further comprise generating, by the one or more processors, a report indicating the presence or absence of the ALK fusion nucleic acid molecule in the sample. In some embodiments, the methods further comprise transmitting the report to a healthcare provider. In some embodiments, the report is transmitted via a computer network or a peer-to-peer connection.

In some embodiments of any of the methods provided herein, the methods further comprise acquiring knowledge of or detecting in a sample from the individual a base substitution, a short insertion/deletion (indel), a copy number alteration, or a genomic rearrangement in one or more genes. In some embodiments, the one or more genes comprise one or more of ALK, ABCB11, ACTN4, AGAP1, APH1A, AZI2, BTBD9, C2orf73, CAPN14, CARMIL1, CASP8, CDC42BPA, CIB4, CNTNAP5, COL3A1, CPQ, CPSF7, CREBBP, CTBP1, CTNND1, CYP51A1, CYS1, EPHA2, FHOD3, FILIP1L, GMCL1, GPN1, GPR113, HADHA, HS1BP3, INTS9, ITGA6, KCTD18, KIF5C, KLC4, LINC00535, LRRFIP2, MAGOHB, MAMDC4, MANBA, MAP3K9, MED13L, METTL25, MTBP, MYH10, MYO5C, NFIA, NINJ2, OPRM1, OTX1, PAQR4, PDCD10, PDE3A, PELI1, PLEC, PTGER4, PTPRJ, QKI, RPS6KA5, SASH1, SEC16B, SKAP1, SLC25A13, SLC30A6, SNX17, SOX13, SRSF7, TANGO6, TG, TMCO3, TNS3, TRIM24, TTC28, UBE2L3, UBE3B, UTRN, VASP, WDR92, YPEL5, ZNF446, ZNF454, ZNF513, or ZSWIM2, or a gene listed in Table 1, or a panel of known/suspected oncogenes and/or tumor suppressors, or any combination thereof.

The disclosed methods may be used with any of a variety of samples, e.g., as described in further detail below. For example, in some instances, the sample may comprise a tissue biopsy sample, a liquid biopsy sample, or a normal control. In some instances, the sample may be a liquid biopsy sample and may comprise blood, plasma, cerebrospinal fluid, sputum, stool, urine, or saliva. In some instances, the sample may be a liquid biopsy sample and may comprise circulating tumor cells (CTCs). In some instances, the sample may be a liquid biopsy sample and may comprise cell-free DNA (cfDNA), circulating tumor DNA (ctDNA), or any combination thereof.

In some instances, the nucleic acid molecules extracted from a sample may comprise a mixture of tumor or cancer nucleic acid molecules and non-tumor or non-cancer nucleic acid molecules. In some instances, the tumor nucleic acid molecules may be derived from a tumor portion of a heterogeneous tissue biopsy sample, and the non-tumor nucleic acid molecules may be derived from a normal portion of the heterogeneous tissue biopsy sample. In some instances, the sample may comprise a liquid biopsy sample, and the tumor or cancer nucleic acid molecules may be derived from a circulating tumor DNA (ctDNA) fraction of the liquid biopsy sample while the non-tumor or non-cancer nucleic acid molecules may be derived from a non-tumor or non-cancer, cell-free DNA (cfDNA) fraction of the liquid biopsy sample. In some embodiments of any of the methods provided herein, the method further comprises determining the circulating tumor DNA (ctDNA) fraction of a liquid biopsy sample.

### (ii) Detection of Fusion Polypeptides

Also provided herein are methods of detecting an ALK fusion polypeptide of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion polypeptide described herein), or a fragment thereof.

An ALK fusion polypeptide provided herein, or a fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, an ALK fusion polypeptide provided herein, or a fragment thereof, can be distinguished from a reference polypeptide, e.g., a non-mutant or wild type protein or polypeptide, with an antibody or antibody fragment that reacts differentially with a mutant protein or polypeptide (e.g., an ALK fusion polypeptide provided herein or a fragment thereof) as compared to a reference protein or polypeptide. In some embodiments, an ALK fusion polypeptide of the disclosure, or a fragment thereof, can be distinguished from a reference polypeptide, e.g., a non-mutant or wild type protein or polypeptide, by reaction with a detection reagent, e.g., a substrate, e.g., a substrate for catalytic activity, e.g., phosphorylation.

In some aspects, methods of detection of an ALK fusion polypeptide of the disclosure, or a fragment thereof, are provided, comprising contacting a sample, e.g., a sample described herein, comprising an ALK fusion polypeptide described herein, with a detection reagent provided herein (e.g., an antibody of the disclosure), and determining if the ALK fusion polypeptide is present in the sample.

(iii) Detection Reagents

In some aspects, provided herein are reagents for detecting an ALK fusion nucleic acid molecule of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein), or a fragment thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid molecule, e.g., a nucleic acid molecule that is or comprises an ALK fusion nucleic acid molecule described herein or a fragment or portion thereof.

In other aspects, provided herein are reagents for detecting an ALK fusion polypeptide of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion polypeptide described herein), or a fragment thereof, e.g., according to the methods of detection provided herein. In some embodiments, a detection reagent provided herein comprises an antibody or antibody fragment that specifically binds to an ALK fusion polypeptide of the disclosure, or to a fragment thereof.

### Baits

In some embodiments, nucleic acids corresponding to a gene involved in an ALK fusion nucleic acid molecule described herein, e.g., an ALK gene, and/or a corresponding gene fusion partner as described herein (e.g., in Tables 1-7, and/or in the Examples herein), are captured (e.g., from amplified nucleic acids) by hybridization with a bait molecule. Provided herein are bait molecules suitable for the detection of an ALK fusion nucleic acid molecule of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein).

In some embodiments, a bait molecule comprises a capture nucleic acid molecule configured to hybridize to a target nucleic acid molecule comprising an ALK fusion nucleic acid molecule of the disclosure, or a fragment or portion thereof. In some embodiments, the capture nucleic acid molecule is configured to hybridize to the ALK fusion nucleic acid molecule of the target nucleic acid molecule.

In some embodiments, the capture nucleic acid molecule is configured to hybridize to a fragment of an ALK fusion nucleic acid molecule of the disclosure. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the fragment comprises a breakpoint or fusion junction of an ALK fusion nucleic acid molecule of the disclosure.

In some embodiments, the capture nucleic acid molecule comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is configured to hybridize to a breakpoint of an ALK fusion nucleic acid molecule of the disclosure, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the capture nucleic acid molecule is configured to hybridize to a nucleotide sequence in an intron or an exon of an ALK gene, or in a breakpoint joining the introns or exons of an ALK gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides), and/or to an intron or exon of another gene (e.g., a corresponding gene fusion partner as described herein, e.g., in Tables 1-7, and/or in the Examples herein).

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label, a tag or detection reagent. In some embodiments, the label, tag or detection reagent is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag or reagent, e.g., that allows capture and isolation of a hybrid formed by a bait and a nucleic acid molecule hybridized to the bait. In some embodiments, the affinity tag or reagent is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

In some embodiments, a bait provided herein hybridizes to a nucleotide sequence corresponding to an intron or an exon of one gene of an ALK fusion molecule described herein (e.g., an ALK gene), in an intron or an exon of the other gene of an ALK fusion molecule described herein (e.g., a corresponding gene fusion partner as described herein, e.g., in any of Tables 1-7, and/or in the Examples herein), and/or a breakpoint joining the introns and/or exons.

The baits described herein can be used for selection of exons and short target sequences.

In some embodiments, a bait of the disclosure distinguishes a nucleic acid molecule, e.g., a genomic or transcribed nucleic acid molecule, e.g., a cDNA or RNA, having a breakpoint of an ALK fusion nucleic acid molecule described herein from a reference nucleotide sequence, e.g., a nucleotide sequence not having the breakpoint.

In some embodiments, the bait hybridizes to a breakpoint of an ALK fusion nucleic acid molecule described herein and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Probes

Also provided herein are probes, e.g., nucleic acid molecules, suitable for the detection of an ALK fusion nucleic acid molecule of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein). In some embodiments, a probe provided herein comprises a nucleic acid sequence configured to hybridize to a target nucleic acid molecule that is or comprises an ALK fusion nucleic acid molecule of the disclosure, or a fragment or portion thereof. In some embodiments, the probe comprises a nucleic acid sequence configured to hybridize to the ALK fusion nucleic acid molecule of the disclosure, or the fragment or portion thereof, of the target nucleic acid molecule. In some embodiments, the probe comprises a nucleic acid sequence configured to hybridize to a fragment or portion of the ALK fusion nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a breakpoint of an ALK fusion nucleic acid molecule of the disclosure, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the breakpoint.

In some embodiments, the probe comprises a nucleotide sequence configured to hybridize to a nucleotide sequence in an intron or an exon of a gene involved in an ALK fusion nucleic acid molecule described herein, e.g., an ALK gene, or in a breakpoint joining the introns or exons of the gene (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides), and/or to an intron or exon of another gene (e.g., a corresponding gene fusion partner as described herein, e.g., in any of Tables 1-7, and/or in the Examples herein).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid molecule hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of ALK fusion nucleic acid molecules provided herein. For example, a probe provided herein may be used for detecting an ALK fusion nucleic acid molecule of the disclosure in a sample, e.g., a sample obtained from an individual. In some embodiments, the probe may be used for identifying cells or tissues that express an ALK fusion nucleic acid molecule of the disclosure, e.g., by measuring levels of the ALK fusion nucleic acid molecule. In some embodiments, the probe may be used for detecting levels of an ALK fusion nucleic acid molecule of the disclosure, e.g., mRNA levels, in a sample of cells from an individual.

In some embodiments, a probe provided herein specifically hybridizes to a nucleic acid molecule comprising a rearrangement (e.g., a deletion, inversion, insertion, duplication, or other rearrangement) resulting in an ALK fusion nucleic acid molecule of the disclosure.

In some embodiments, a probe of the disclosure distinguishes a nucleic acid, e.g., a genomic or transcribed nucleic acid, e.g., a cDNA or RNA, having a breakpoint of an ALK fusion nucleic acid molecule of the disclosure, from a reference nucleotide sequence, e.g., a nucleotide sequence not having the breakpoint.

Also provided herein are isolated pairs of allele-specific probes, wherein, for example, the first probe of the pair specifically hybridizes to an ALK fusion nucleic acid molecule of the disclosure, and the second probe of the pair specifically hybridizes to a corresponding wild type sequence. Probe pairs can be designed and produced for any of the ALK fusion nucleic acid molecules described herein and are useful in detecting a somatic mutation in a sample. In some embodiments, a first probe of a pair specifically hybridizes to a mutation (e.g., the breakpoint of an alteration, rearrangement, inversion, duplication, deletion, insertion or translocation resulting in an ALK fusion nucleic acid molecule described herein), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 10′ nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA that is or comprises an ALK fusion nucleic acid molecule of the disclosure.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in an ALK fusion nucleic acid molecule of the disclosure. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations, reciprocal translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules may be used, or further processing may be performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to a breakpoint of an ALK fusion nucleic acid molecule of the disclosure, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule that is or comprises an ALK fusion nucleic acid molecule of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYOSC, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein), or a fragment or portion thereof. In some embodiments, the oligonucleotide comprises a nucleotide sequence configured to hybridize to the ALK fusion nucleic acid molecule of the target nucleic acid molecule. In some embodiments, the oligonucleotide comprises a nucleotide sequence configured to hybridize to a fragment or portion of the ALK fusion nucleic acid molecule of the target nucleic acid molecule.

In some embodiments, the oligonucleotide, e.g., the primer, comprises a nucleotide sequence configured to hybridize to a breakpoint of an ALK fusion nucleic acid molecule of the disclosure, and may be further configured to hybridize to between about 10 and about 12, about 12 and about 15, about 15 and about 17, about 17 and about 20, about 20 and about 25, or about 25 and about 30, or more nucleotides flanking either side of the breakpoint.

In some embodiments, the oligonucleotide, e.g., the primer, comprises a nucleotide sequence configured to hybridize to a nucleotide sequence in an intron or an exon of a gene involved in an ALK fusion nucleic acid mole of the disclosure (e.g., an ALK gene), to a breakpoint of a fusion nucleic acid molecule described herein, and/or to an intron or exon of another gene (e.g., a corresponding gene fusion partner as described herein, e.g., in any of Tables 1-7, and/or in the Examples herein).

In some embodiments, the oligonucleotide comprises a nucleotide sequence corresponding to an ALK fusion nucleic acid molecule of the disclosure. In some embodiments, the oligonucleotide comprises a nucleotide sequence corresponding to a fragment or a portion of the ALK fusion nucleic acid molecule. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to an ALK fusion nucleic acid molecule provided herein. In some embodiments, the oligonucleotide comprises a nucleotide sequence complementary to a fragment or a portion of the ALK fusion nucleic acid molecule provided herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule that is or comprises an ALK fusion nucleic acid molecule of the disclosure, or a fragment thereof, e.g., using PCR. In some embodiments, the oligonucleotide may be used to sequence a nucleic acid molecule that is or comprises an ALK fusion nucleic acid molecule provided herein, or a fragment thereof. In some embodiments, the oligonucleotide may be used to amplify a nucleic acid molecule comprising a breakpoint of an ALK fusion nucleic acid molecule described herein, e.g., using PCR. In some embodiments, the oligonucleotide may be used to sequence a nucleic acid molecule comprising a breakpoint of an ALK fusion nucleic acid molecule described herein.

In some embodiments, pairs of oligonucleotides, e.g., pairs of primers, are provided herein, which are configured to hybridize to a nucleic acid molecule that is or comprises an ALK fusion nucleic acid molecule of the disclosure, or a fragment thereof. In some embodiments, a pair of oligonucleotides of the disclosure may be used for directing amplification of the ALK fusion nucleic acid molecule or fragment thereof, e.g., using a PCR reaction. In some embodiments, pairs of oligonucleotides, e.g., pairs of primers, are provided herein, which are configured to hybridize to a nucleic acid molecule comprising a breakpoint of an ALK fusion nucleic acid molecule described herein, e.g., for use in directing amplification of the corresponding fusion nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., an ALK fusion nucleic acid molecule of the disclosure, or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having a breakpoint of an ALK fusion nucleic acid molecule described herein, from a reference nucleotide sequence, e.g., a nucleotide sequence not having the breakpoint.

In one aspect, provided herein is a primer or primer set for amplifying a nucleic acid molecule comprising a cytogenetic abnormality such as an alteration, rearrangement, chromosomal inversion, deletion, translocation, duplication, or other rearrangement resulting in an ALK fusion nucleic acid molecule of the disclosure. In another aspect, provided herein is a primer or primer set for amplifying a nucleic acid molecule comprising an alteration, rearrangement, chromosomal inversion, insertion, deletion, translocation, duplication or other rearrangement resulting in an ALK fusion nucleic acid molecule of the disclosure. In certain aspects, provided herein are allele-specific oligonucleotides, e.g., primers, wherein a first oligonucleotide of a pair specifically hybridizes to a mutation (e.g., a breakpoint of an ALK fusion nucleic acid molecule described herein), and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., a breakpoint of an ALK fusion nucleic acid molecule described herein), and a second oligonucleotide of the pair specifically hybridizes to a sequence downstream of the mutation.

In some embodiments, the oligonucleotide, e.g., the primer, hybridizes to a breakpoint of an ALK fusion nucleic acid molecule described herein, and a sequence on either side of the breakpoint (e.g., any of 1, 2, 3, 4, 5, 6, 7, 8, 9, or 10 nucleotides on either side of the breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the breakpoint).

### Antibodies

Provided herein are antibodies or antibody fragments that specifically bind to an ALK fusion polypeptide of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion polypeptide described herein), or a fragment thereof.

The antibody may be of any suitable type of antibody, including, but not limited to, a monoclonal antibody, a polyclonal antibody, a multi-specific antibody (e.g., a bispecific antibody), or an antibody fragment, so long as the antibody or antibody fragment exhibits a specific antigen binding activity, e.g., binding to an ALK fusion polypeptide of the disclosure, or a fragment thereof.

In some embodiments, a fusion polypeptide of the disclosure, or a fragment thereof, is used as an immunogen to generate one or more antibodies of the disclosure, e.g., using standard techniques for polyclonal and monoclonal antibody preparation. In some embodiments, an ALK fusion polypeptide provided herein, is used to provide antigenic peptide fragments (e.g., comprising any of at least about 8, at least about 10, at least about 15, at least about 20, at least about 30 or more amino acids) for use as immunogens to generate one or more antibodies of the disclosure, e.g., using standard techniques for polyclonal and monoclonal antibody preparation. As is known in the art, an antibody of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., an ALK fusion polypeptide of the disclosure, or a fragment thereof. The preparation can further include an adjuvant, such as Freund's complete or incomplete adjuvant, or a similar immunostimulatory agent.

In some embodiments, an antibody provided herein is a polyclonal antibody. Methods of producing polyclonal antibodies are known in the art. In some embodiments, an antibody provided herein is a monoclonal antibody, wherein a population of the antibody molecules contain only one species of an antigen binding site capable of immunoreacting or binding with a particular epitope, e.g., an epitope on an ALK fusion polypeptide provided herein. Methods of preparation of monoclonal antibodies are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody of the disclosure may also be identified and isolated by screening a recombinant combinatorial immunoglobulin library (e.g., an antibody phage display library) with the polypeptide of interest, e.g., an ALK fusion polypeptide provided herein or a fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies of the disclosure are recombinant antibodies, such as chimeric and humanized monoclonal antibodies, comprising both human and non-human portions. Such chimeric and/or humanized monoclonal antibodies can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody of the disclosure is a human monoclonal antibody. In some embodiments, human monoclonal antibodies are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies, and protocols for producing such antibodies, see, e.g., U.S. Pat. Nos. 5,625,126; 5,633,425; 5,569,825; 5,661,016; and U.S. Pat. No. 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody of the disclosure can be used to isolate an ALK fusion polypeptide provided herein, or a fragment thereof, by standard techniques, such as affinity chromatography or immunoprecipitation. In some embodiments, an antibody of the disclosure can be used to detect an ALK fusion polypeptide provided herein, or a fragment thereof, e.g., in a tissue sample, cellular lysate, or cell supernatant, in order to evaluate the level and/or pattern of expression of the fusion polypeptide. Detection can be facilitated by coupling the antibody to a detectable substance. Thus, in some embodiments, an antibody of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ²I, ¹³¹I, ³⁵S or ³H.

An antibody or antibody fragment of the disclosure may also be used diagnostically, e.g., to detect and/or monitor protein levels (e.g., protein levels of an ALK fusion polypeptide provided herein) in tissues or body fluids (e.g., in a tumor cell-containing tissue or body fluid), e.g., according to the methods provided herein.

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g., 10⁻⁸M or less, e.g., from 10⁻⁸ M to 10⁻¹³ M, e.g., from 10⁻⁹ M to 10⁻¹³ M). Methods of measuring antibody affinity (e.g., Kd) are known in the art, and include, without limitation, a radiolabeled antigen binding assay (RIA) and a BIACORE® surface plasmon resonance assay. In some embodiments, antibody affinity (e.g., Kd) is determined using the Fab version of an antibody of the disclosure and its antigen (e.g., an ALK fusion polypeptide provided herein). In some embodiments, a RIA is performed with the Fab version of an antibody of the disclosure and its antigen (e.g., an ALK fusion polypeptide provided herein).

In certain embodiments, an antibody provided herein is an antibody fragment. Antibody fragments include, but are not limited to, Fab, Fab′, Fab′-SH, F(ab′)₂, Fv, and single-chain antibody molecule (e.g., scFv) fragments, and other fragments described herein or known in the art.

In certain embodiments, an antibody provided herein is a diabody. Diabodies are antibody fragments with two antigen-binding sites that may be bivalent or bispecific. In certain embodiments, an antibody provided herein is a triabody or a tetrabody.

In certain embodiments, an antibody provided herein is a single-domain antibody. Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody, as well as production by recombinant host cells (e.g., E. coli or phage), as known in the art and as described herein.

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey), and a human constant region. In a further example, a chimeric antibody is a “class switched” antibody, in which the class or subclass of the antibody has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof), are derived from a non-human antibody, and framework regions (FRs) (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity. Humanized antibodies and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

In certain embodiments, an antibody provided herein is a human antibody. Human antibodies can be produced using various techniques known in the art. For example, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies can also be made by hybridoma-based methods known in the art, e.g., using known human myeloma and mouse-human heteromyeloma cell lines for the production of human monoclonal antibodies. Human antibodies may also be generated by isolating Fv clone variable domain sequences selected from human-derived phage display libraries. Such variable domain sequences may then be combined with a desired human constant domain. Techniques for selecting human antibodies from antibody libraries are known in the art and described herein.

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies to a wide range of non-self and also self antigens without any immunization. Naive libraries can also be made synthetically by cloning un-rearranged V-gene segments from stem cells, and using PCR primers containing random sequences to amplify the highly variable CDR3 regions and to accomplish rearrangement in vitro. Antibodies or antibody fragments isolated from human antibody libraries are considered human antibodies or human antibody fragments herein.

In certain embodiments, an antibody provided herein is a multispecific antibody, e.g., a bispecific antibody. Multispecific antibodies are monoclonal antibodies that have binding specificities for at least two different sites or at least two different antigens. For example, one of the binding specificities can be to an ALK fusion polypeptide of the disclosure, and the other can be to any other antigen. Multispecific antibodies can be prepared as full length antibodies or as antibody fragments. Techniques for making multispecific antibodies are known in the art and include, but are not limited to, recombinant co-expression of two immunoglobulin heavy chain-light chain pairs having different specificities, and “knob-in-hole” engineering. Multispecific antibodies may also be made by engineering electrostatic steering effects (e.g., by introducing mutations in the constant region) for making heterodimeric Fes; cross-linking two or more antibodies or fragments; using leucine zippers to produce bispecific antibodies; using “diabody” technology for making bispecific antibody fragments; using single-chain Fv (scFv) dimers; and preparing trispecific antibodies. Engineered antibodies with three or more functional antigen binding sites, including “Octopus antibodies,” are also included in the disclosure. Antibodies or antibody fragments of the disclosure also include “Dual Acting FAbs” or “DAF,” e.g., comprising an antigen binding site that binds to an ALK fusion polypeptide of the disclosure as well as another, different antigen.

In certain embodiments, amino acid sequence variants of the antibodies provided herein are contemplated. For example, it may be desirable to improve the binding affinity and/or other biological properties of the antibody. Amino acid sequence variants of an antibody of the disclosure may be prepared by introducing appropriate modifications into the nucleotide sequence encoding the antibody, or by peptide synthesis. Such modifications include, for example, deletions, and/or insertions, and/or substitutions of residues within the amino acid sequences of the antibody. Any combination of deletions, insertions, and substitutions can be made to arrive at the final antibody, provided that the final antibody possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody of the present disclosure is altered to increase or to decrease the extent to which the antibody is glycosylated. Addition or deletion of glycosylation sites to an antibody may be conveniently accomplished by altering the amino acid sequence of the antibody, such that one or more glycosylation sites is created or removed. Antibody variants having bisected oligosaccharides are further provided, e.g., in which a biantennary oligosaccharide attached to the Fc region of the antibody is bisected by GlcNAc. In some embodiments, antibody variants of the disclosure may have increased fucosylation. In some embodiments, antibody variants of the disclosure may have reduced fucosylation. In some embodiments, antibody variants of the disclosure may have improved ADCC function. In some embodiments, antibody variants of the disclosure may have decreased ADCC function. Antibody variants with at least one galactose residue in the oligosaccharide attached to the Fc region are also provided. Such antibody variants may have improved CDC function. In some embodiments, antibody variants of the disclosure may have increased CDC function. In some embodiments, antibody variants of the disclosure may have decreased CDC function.

In certain embodiments, one or more amino acid modifications may be introduced into the Fc region of an antibody of the present disclosure, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody variants with improved or diminished binding to FcRs are also included in the disclosure. In certain embodiments, an antibody variant comprises an Fc region with one or more amino acid substitutions that improve ADCC, e.g., substitutions at positions 298, 333, and/or 334 of the Fc region. In some embodiments, numbering of Fc region residues is according to EU numbering of residues. In some embodiments, alterations are made in the Fc region that result in altered (i.e., either improved or diminished) C1q binding and/or CDC. In some embodiments, antibodies of the disclosure include antibodies with increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody provided herein is a cysteine-engineered antibody, e.g., “thioMAb,” in which one or more residues of the antibody are substituted with cysteine residues. In some embodiments, the substituted residues occur at accessible sites of the antibody. By substituting those residues with cysteine, reactive thiol groups are thereby positioned at accessible sites of the antibody, and may be used to conjugate the antibody to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyamino acids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody may vary, and if more than one polymer is attached, the polymers can be the same or different molecules. In general, the number and/or type of polymers used for derivatization can be determined based on considerations including, but not limited to, the particular properties or functions of the antibody to be improved, or whether the antibody derivative will be used in a therapy under defined conditions. In some embodiments, provided herein are antibodies conjugated to carbon nanotubes, e.g., for use in methods to selectively heat the antibody using radiation to a temperature at which cells proximal to the antibody are killed.

### (iv) Samples

A variety of materials can be the source of, or serve as, samples for use in any of the methods of the disclosure, such as the methods for detection of an ALK fusion nucleic acid molecule or polypeptide of the disclosure, or fragments thereof.

For example, the sample can be, or be derived from: solid tissue such as from a fresh, frozen and/or preserved organ, tissue sample, biopsy (e.g., tumor, tissue or liquid biopsy), resection, smear, or aspirate; scrapings; bone marrow or bone marrow specimens; a bone marrow aspirate; blood or any blood constituents; blood cells; bodily fluids such as cerebrospinal fluid, amniotic fluid, urine, saliva, sputum, peritoneal fluid or interstitial fluid; pleural fluid; ascites; tissue or fine needle biopsy samples; surgical specimens; cell-containing body fluids; free-floating nucleic acids; feces; lymph; gynecological fluids; skin swabs; vaginal swabs; oral swabs; nasal swabs; washings or lavages such as ductal lavages or bronchoalveolar lavages; cells from any time in gestation or development of an individual; cells from a cancer or tumor; other body fluids, secretions, and/or excretions, and/or cells therefrom. In some embodiments, a sample is or comprises cells obtained from an individual. In some embodiments, the sample is or is derived from blood or blood constituents, e.g., obtained from a liquid biopsy. In some embodiments, the sample is or is derived from a tumor sample. In some embodiments, the sample is or comprises biological tissue or fluid. In some embodiments, the sample can contain compounds that are not naturally intermixed with the source of the sample in nature, such as preservatives, anticoagulants, buffers, fixatives, nutrients, antibiotics or the like. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In one embodiment, the sample is or is acquired from a liquid biopsy of blood, plasma, cerebrospinal fluid, sputum, stool, urine, or saliva. In some embodiments, the sample includes cell-free DNA (cfDNA) and/or circulating tumor DNA (ctDNA), e.g., from a biopsy of blood, plasma, cerebrospinal fluid, sputum, stool, urine, or saliva. In another embodiment, the sample includes one or more circulating tumor cells (CTCs) (e.g., a CTC acquired from a blood sample). In one embodiment, the sample is a cell not associated with a tumor or cancer, e.g., a non-tumor or non-cancer cell or a peripheral blood lymphocyte.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, nucleic acids (e.g., for use in any of the methods for detection of ALK fusion nucleic acid molecules provided herein) or proteins (e.g., for use in any of the methods for detection of ALK fusion polypeptides provided herein) extracted from a sample or obtained by subjecting a primary sample to techniques such as amplification methods, reverse transcription of mRNA, or isolation and/or purification of certain components such as nucleic acids and/or proteins.

In some embodiments, the sample comprises nucleic acids, e.g., genomic DNA, cDNA, or mRNA. In some embodiments, the sample comprises cell-free DNA (cfDNA). In some embodiments, the sample comprises cell-free RNA (cfRNA). In some embodiments, the sample comprises circulating tumor DNA (ctDNA). In certain embodiments, the nucleic acids are purified or isolated (e.g., removed from their natural state). In some embodiments, the sample comprises tumor or cancer nucleic acids, such as nucleic acids from a tumor or cancer sample, e.g., genomic DNA, RNA, or cDNA derived from RNA, or from a liquid biopsy, e.g., ctDNA from blood, plasma, cerebrospinal fluid, sputum, stool, urine, or saliva. In certain embodiments, a tumor or cancer nucleic acid sample, or a ctDNA sample, is purified or isolated (e.g., it is removed from its natural state).

In some embodiments, the sample comprises tumor or cancer proteins or polypeptides, such as proteins or polypeptides from a tumor or a cancer sample, or from a liquid biopsy, e.g., from blood, plasma, cerebrospinal fluid, sputum, stool, urine, or saliva. In certain embodiments, the proteins or polypeptides are purified or isolated (e.g., removed from their natural state).

In some embodiments, the sample is obtained from an individual having a cancer, such as a cancer described herein. In some embodiments, the sample comprises an ALK fusion nucleic acid molecule or polypeptide of the disclosure.

In some embodiments, the sample is a control sample or a reference sample, e.g., not containing an ALK fusion nucleic acid molecule or polypeptide described herein. In certain embodiments, the reference sample is purified or isolated (e.g., it is removed from its natural state). In certain embodiments, the reference or control sample comprises a wild type or a non-mutated nucleic acid molecule or polypeptide counterpart to any of the ALK fusion nucleic acid molecules or polypeptides described herein. In other embodiments, the reference sample is from a non-tumor or cancer sample, e.g., a blood control, a normal adjacent tumor (NAT), or any other non-cancerous sample from the same or a different individual.

In some embodiments, an ALK fusion nucleic acid molecule of the disclosure is detected in a sample comprising genomic or subgenomic DNA fragments, or RNA (e.g., mRNA), isolated from a sample, e.g., a tumor or cancer sample, a normal adjacent tissue (NAT) sample, a tissue sample, or a blood, plasma, cerebrospinal fluid, sputum, stool, urine, or saliva sample obtained from an individual.

In some embodiments, the sample comprises cDNA derived from an mRNA sample or from a sample comprising mRNA. In some embodiments, an ALK fusion nucleic acid molecule of the disclosure is detected in a sample comprising cell-free DNA (cfDNA), cell-free RNA, and/or circulating tumor DNA (ctDNA). In some embodiments, an ALK fusion nucleic acid molecule of the disclosure is detected in a sample comprising cell-free DNA (cfDNA) and/or circulating tumor DNA (ctDNA). In some embodiments, an ALK fusion nucleic acid molecule of the disclosure is detected in a sample comprising circulating tumor DNA (ctDNA).

### C. Anti-Cancer Therapies

Certain aspects of the present disclosure relate to anti-cancer therapies, as well as methods for identifying an individual having a cancer who may benefit from a treatment comprising an anti-cancer therapy; selecting a treatment for an individual having a cancer; identifying one or more treatment options for an individual having a cancer; predicting survival of an individual having a cancer; treating or delaying progression of cancer; monitoring, evaluating or screening an individual having a cancer; detecting the presence or absence of a cancer in an individual; monitoring progression or recurrence of a cancer in an individual; or identifying a candidate treatment for a cancer in an individual in need thereof. The present disclosure also provides uses for anti-cancer therapies (e.g., in methods of treating or delaying progression of cancer in an individual, or in methods for manufacturing a medicament for treating or delaying progression of cancer). In some instances, the methods of the disclosure can include administering an anti-cancer therapy or applying an anti-cancer therapy to an individual based on a generated genomic and/or sequencing mutation profile. An anti-cancer therapy can refer to a compound that is effective in the treatment of cancer cells. Examples of anti-cancer agents or anti-cancer therapies include, but not limited to, alkylating agents, antimetabolites, natural products, hormones, chemotherapy, radiation therapy, immunotherapy, surgery, or a therapy configured to target a defect in a specific cell signaling pathway, e.g., a defect in a DNA mismatch repair (MMR) pathway.

In some embodiments, an anti-cancer therapy of the disclosure is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer comprising an ALK fusion nucleic acid molecule or polypeptide of the disclosure, a treatment for cancer being tested in a clinical trial, a targeted therapy, a treatment being tested in a clinical trial for cancer comprising an ALK fusion nucleic acid molecule or polypeptide of the disclosure, or any combination thereof, e.g., a described in further detail below. In some embodiments, the anti-cancer therapy is an ALK-targeted therapy. In some embodiments, the anti-cancer therapy is a kinase inhibitor, such as a kinase inhibitor described herein or known in the art. In some embodiments, the kinase inhibitor is a multi-kinase inhibitor or an ALK-specific inhibitor known in the art or described herein. In some embodiments, the nucleic acid inhibits the expression of an ALK fusion nucleic acid molecule or polypeptide of the disclosure.

In some embodiments, an anti-cancer therapy of the disclosure is an ALK-targeted therapy, e.g., as described herein or known in the art. In some embodiments, the ALK-targeted therapy is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof. In some embodiments, the ALK-targeted therapy is a kinase inhibitor known in the art or described herein. In some embodiments, the ALK-targeted therapy is a tyrosine kinase inhibitor known in the art or described herein. In some embodiments, the ALK-targeted therapy is a multi-kinase inhibitor or an ALK-specific inhibitor known in the art or described herein. In some embodiments, the kinase inhibitor inhibits the kinase activity of an ALK polypeptide. In some embodiments, the ALK-targeted therapy comprises one or more of crizotinib, alectinib (AF802, CH5424802), ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A. In some embodiments, the nucleic acid inhibits the expression of an ALK fusion nucleic acid molecule or polypeptide of the disclosure. In some embodiments, the ALK-targeted therapy is an ALK kinase inhibitor, e.g., as described in examples 3-39 of WO2005016894, which is incorporated herein by reference.

In some embodiments, the ALK-targeted therapy comprises alectinib. In a Phase 3 study comparing alectinib with crizotinib in ALK-rearranged, inhibitor-naive NSCLC, patients treated with alectinib experienced significantly improved median PFS (34.8 vs. 10.9 months; HR=0.43), with PFS benefit observed in patients with EML4-ALK variants 1, 2, and 3a/b (Peters et al., The New England journal of medicine, vol. 377, 9 (2017): 829-838; Camidge et al., Journal of thoracic oncology, vol. 14, 7 (2019): 1233-1243). Similar results have been reported in the J-ALEX trial for inhibitor-naive Japanese patients with ALK-positive NSCLC (Hida et al., Lancet, vol. 390, 10089 (2017): 29-39; Nakagawa et al., Lung cancer, vol. 139 (2020): 195-199). For patients with crizotinib-refractory ALK-rearranged NSCLC, Phase 1/2 and Phase 2 trials of alectinib reported ORRs of 45% to 55% (Shaw et al., The Lancet. Oncology, vol. 17, 2 (2016): 234-242; Gadgeel et al., The Lancet. Oncology, vol. 15, 10 (2014): 1119-28; Ou et al., Journal of clinical oncology, vol. 34, 7 (2016): 661-8), and the Phase 3 ALUR trial showed that alectinib significantly improved PFS compared with chemotherapy (7.1 vs. 1.6 months; HR=0.32) (Novello et al., Annals of oncology, vol. 29, 6 (2018): 1409-1416). Alectinib combined with atezolizumab led to an ORR of 81% (17/21) as first-line treatment for PD-L1 unselected, ALK+NSCLC (Kim et al., Journal of Clinical Oncology 2018 36:15_suppl, 9009-9009). Accordingly, an individual having a cancer comprising any of the ALK fusion nucleic acid molecules or polypeptides provided herein may benefit from a treatment comprising alectinib.

In some embodiments, the ALK-targeted therapy comprises brigatinib. An initial Phase 1/2 study of brigatinib for the treatment of patients with ALK-rearranged NSCLC reported responses in 71.8% (51/71) of crizotinib-pretreated patients and in 100.0% (8/8) of crizotinib-naive patients with median PFSs of 13.2 months and unreached, respectively (Gettinger et al., The Lancet. Oncology, vol. 17, 12 (2016): 1683-1696). Interim analyses of the Phase 3 ALTA-1L study comparing brigatinib to crizotinib for front-line treatment of patients with ALK-positive NSCLC show a superior median PFS (24.0 vs. 11.0 months, HR=0.49) and a significantly higher intracranial ORR (78% vs. 30%) with brigatinib (Camidge et al., The New England journal of medicine, vol. 379, 21 (2018): 2027-2039; Camidge et al., Annals of Oncology (2019) 30 (suppl_9): ix183-ix202). The Phase 2 ALTA study demonstrated the activity of brigatinib with 2 different dosing regimens following progression on crizotinib for patients with ALK-positive NSCLC, reporting ORRs of 46% and 56%, median PFSs of 9.2 and 16.7 months, and median OSs of 29.5 and 34.1 months (Huber et al., Journal of thoracic oncology, vol. 15, 3 (2020): 404-415). A retrospective study reported an ORR of 16.7% (3/18), a DCR of 66.7% (12/18), and a median PFS of 4.4 months for patients with ALK-positive alectinib-refractory NSCLC treated with brigatinib (Lin et al., Journal of thoracic oncology, vol. 13, 10 (2018): 1530-1538). Accordingly, an individual having a cancer comprising any of the ALK fusion nucleic acid molecules or polypeptides provided herein may benefit from a treatment comprising brigatinib.

In some embodiments, the ALK-targeted therapy comprises ceritinib. A Phase 3 study of ceritinib for ALK inhibitor-naive patients with ALK-rearranged NSCLC observed a whole-body (WB) ORR of 63.7%, a WB DCR of 89.5%, and PFS of 11.1 months (Felip et al., Journal of Clinical Oncology 2015 33:15_suppl, 8060-8060). Following progression on prior chemotherapy and crizotinib, patients with ALK-rearranged NSCLC achieved a WB ORR of 38.6%, WB DCR of 77.1%, and PFS of 5.4 months on ceritinib (Mok et al., Journal of Clinical Oncology 2015 33:15_suppl, 8059-8059). A Phase 1 study of ceritinib reported a 58% response rate for 114 patients with ALK-rearranged NSCLC and a response rate of 56% for 80 of these patients who had previously been treated with crizotinib (Shaw et al., The New England journal of medicine, vol. 370, 13 (2014): 1189-97). A patient with colorectal carcinoma and a STRN-ALK fusion exhibited clinical benefit from ceritinib (Yakirevich et al., Clinical cancer research, vol. 22, 15 (2016): 3831-40). A patient with pancreatic ductal adenocarcinoma harboring an EML4-ALK fusion was reported to have a response lasting 2 months (Singhi et al., 2016; USCAP Abstract 1775). Accordingly, an individual having a cancer comprising any of the ALK fusion nucleic acid molecules or polypeptides provided herein may benefit from a treatment comprising ceritinib.

In some embodiments, the ALK-targeted therapy comprises crizotinib. Three Phase 3 studies for patients with ALK-positive NSCLC reported superior PFS (7.7-11.1 vs. 3.0-7.0 months) and ORR (65.0%-87.5% vs. 20.0%-45.6%) when treated with crizotinib compared with chemotherapy regimens in various settings (Lu et al., Journal of Clinical Oncology 2016 34:15_suppl, 9058-9058; Shaw et al., Journal of Clinical Oncology 2016 34:15_suppl, 9066-9066; Solomon et al., The New England journal of medicine, vol. 371, 23 (2014): 2167-77). A pooled retrospective analysis of patients with ALK-rearranged NSCLC and concurrent brain metastases from the PROFILE 1007 and 1005 studies reported 12-week intracranial DCRs of 56% versus 62% and intracranial ORRs of 18% versus 33% for patients with previously untreated versus previously treated brain metastases (Costa et al., Journal of clinical oncology, vol. 33, 17 (2015): 1881-8). In a retrospective study of 90 patients with brain metastases from ALK-rearranged NSCLC, the median OS after diagnosis of brain metastasis was 49.5 months; lack of prior targeted therapy, absence of extracranial metastasis, and a Karnofsky performance score of 90 or higher were significantly associated with improved OS (Johung et al., Journal of clinical oncology, vol. 34, 2 (2016): 123-9). Upon disease progression, further survival benefit has been observed for patients with ALK-positive NSCLC who continue crizotinib treatment (Ou et al., Annals of oncology, vol. 25, 2 (2014): 415-22). Accordingly, an individual having a cancer comprising any of the ALK fusion nucleic acid molecules or polypeptides provided herein may benefit from a treatment comprising crizotinib.

In some embodiments, the ALK-targeted therapy comprises entrectinib. Phase 1 trials have reported a combined 66.7% ORR (1 CR, 5 PRs; n=9) for patients with ALK-rearranged solid tumors treated with entrectinib; responses were observed for patients with NSCLC (2/4), renal cell carcinoma (1/1), colorectal cancer ([CRC], 1/1), and inflammatory myofibroblastic tumors (2/2) (Drilon et al., Cancer discovery vol. 7, 4 (2017): 400-409; Desai et al., Journal of Clinical Oncology 2020 38:15_suppl, 107-107). Accordingly, an individual having a cancer comprising any of the ALK fusion nucleic acid molecules or polypeptides provided herein may benefit from a treatment comprising entrectinib.

In some embodiments, the ALK-targeted therapy comprises lorlatinib. In the Phase 3 CROWN study for the first-line treatment of ALK-positive non-small cell lung cancer (NSCLC), lorlatinib was shown to be superior to crizotinib, with significantly improved median PFS (mPFS; not estimable vs. 9.3 months, HR=0.28) and higher overall ORR (76% vs. 58%) and intracranial ORR (82.4% [14/17] vs. 23.1% [3/13]) (Shaw et al., The New England journal of medicine, vol. 383, 21 (2020): 2018-2029). In a pivotal Phase 2 study for patients with ALK-positive NSCLC who had progressed on 1 or more ALK TKIs, lorlatinib elicited a 47% ORR, a 63.0% (51/81) intracranial ORR, and an mPFS of 7.3 months (Solomon et al., The Lancet. Oncology, vol. 19, 12 (2018): 1654-1667); the ORR was 40% for patients previously treated with 1 or more second-generation TKIs (Bauer et al., 2020; AACR Abstract CT025). For patients whose tumors harbored 1 or more ALK kinase domain mutations, lorlatinib led to responses for 64.4% (29/45), including 57.1% (16/28) of those with the ALK G1202R resistance mutation (Shaw et al., Journal of clinical oncology, vol. 37, 16 (2019): 1370-1379); G1202, therefore, does not appear to represent a major mechanism of lorlatinib resistance (Yoda et al., Cancer discovery, vol. 8, 6 (2018): 714-729; Dagogo-Jack, Ibiayi et al., JCO precision oncology, vol. 2018 (2018): PO.17.00160; Ou et al., Lung cancer, vol. 106 (2017): 110-114). In an expansion cohort of a Phase 1/2 study, patients with ALK-positive NSCLC who remained on lorlatinib following progression exhibited improved median OS and median OS post-PD relative to patients who received another treatment or no subsequent treatment (Ou et al., 2020; ESMO Abstract 1302P). In the JAVELIN Lung 101 study, the combination of lorlatinib and avelumab led to an ORR of 46.4% (13/28) for patients with previously treated ALK-positive NSCLC (Shaw et al., Journal of Clinical Oncology 2018 36:15_suppl, 9008-9008). In the fourth-line setting, lorlatinib led to disappearance of leptomeningeal disease for a patient with ALK-rearranged metastatic inflammatory myofibroblastic sarcoma (Yuan et al., The American journal of case reports, vol. 18 799-804. 17 Jul. 2017; Parker et al., Journal of oncology pharmacy practice, vol. 25, 5 (2019): 1226-1230). Accordingly, an individual having a cancer comprising any of the ALK fusion nucleic acid molecules or polypeptides provided herein may benefit from a treatment comprising lorlatinib.

In some embodiments, an anti-cancer therapy of the disclosure (e.g., an ALK-targeted therapy) is administered in combination with an additional anti-cancer therapy. In some embodiments, the additional anti-cancer therapy is any anti-cancer therapy known in the art or described herein. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

In some embodiments, an anti-cancer therapy of the disclosure comprises a cyclin-dependent kinase (CDK) inhibitor, e.g., alone or in combination with an ALK-targeted therapy. In some embodiments, the CDK inhibitor inhibits CDK4. In some embodiments, the CDK inhibitor inhibits Cyclin D/CDK4. In some embodiments, the CDK inhibitor is (a) a small molecule that inhibits one or more enzymatic activities of CDK4, (b) an antibody that inhibits one or more activities of CDK4 (e.g., by binding to and inhibiting one or more activities of CDK4, binding to and inhibiting expression of CDK4, and/or binding to and inhibiting one or more activities of a cell expressing CDK4, such as by inducing antibody-dependent cellular cytotoxicity, ADCC, or phagocytosis, ADCP), or (c) a nucleic acid that inhibits expression of CDK4 (e.g., an antisense oligonucleotide, miRNA, siRNA, morpholino, CRISPR-based therapeutic, and the like). In some embodiments, the CDK inhibitor inhibits CDK4 and CDK6. In some embodiments, the CDK inhibitor is a small molecule inhibitor of CDK4 (e.g., a competitive or non-competitive inhibitor). Non-limiting examples of CDK inhibitors include palbociclib, ribociclib, and abemaciclib, as well as pharmaceutically acceptable salts thereof.

In some embodiments, an anti-cancer therapy of the disclosure comprises a murine double minute 2 homolog (MDM2) inhibitor, e.g., alone or in combination with an ALK-targeted therapy. In some embodiments, the MDM2 inhibitor is (a) a small molecule that inhibits one or more activities of MDM2 (e.g., binding to p53), (b) an antibody that inhibits one or more activities of MDM2 (e.g., by binding to and inhibiting one or more activities of MDM2, binding to and inhibiting expression of MDM2, and/or binding to and inhibiting one or more activities of a cell expressing MDM2, such as by inducing antibody-dependent cellular cytotoxicity, ADCC, or phagocytosis, ADCP), or (c) a nucleic acid that inhibits expression of MDM2 (e.g., an antisense oligonucleotide, miRNA, siRNA, morpholino, CRISPR-based therapeutic, and the like). In some embodiments, the MDM2 inhibitor is a small molecule inhibitor of MDM2 (e.g., a competitive or non-competitive inhibitor). Non-limiting examples of MDM2 inhibitors include nutlin-3a, RG7112, idasanutlin (RG7388), AMG-232, MI-63, MI-291, MI-391, MI-77301 (SAR405838), APG-115, DS-3032b, NVP-CGMO97, and HDM-201 (siremadlin), as well as pharmaceutically acceptable salts thereof. In some embodiments, the MDM2 inhibitor inhibits or disrupts interaction between MDM2 and p53.

In some embodiments, an anti-cancer therapy of the disclosure comprises (alone or in combination with an ALK-targeted therapy) one or more of an antimetabolite, DNA-damaging agent, or platinum-containing therapeutic (e.g., 5-azacitadine, 5-fluorouracil, acadesine, busulfan, carboplatin, cisplatin, chlorambucil, CPT-11, cytarabine, daunorubicin, decitabine, doxorubicin, etoposide, fludarabine, gemcitabine, idarubicin, radiation, oxaliplatin, temozolomide, topotecan, trabectedin, GSK2830371, or rucaparib); a pro-apoptotic agent (e.g., a BCL2 inhibitor or downregulator, SMAC mimetic, or TRAIL agonist such as ABT-263, ABT-737, oridonin, venetoclax, combination of venetoclax and an anti-CD20 antibody such as obinutuzumab or rituximab, 1396-11, ABT-10, SM-164, D269H/E195R, or rhTRAIL); a tyrosine kinase inhibitor (e.g., as described herein); an inhibitor of RAS, RAF, MEK, or the MAPK pathway (e.g., AZD6244, dabrafenib, LGX818, PD0325901, pimasertib, trametinib, or vemurafenib); an inhibitor of PI3K, mTOR, or Akt (e.g., as described herein); a CDK inhibitor (e.g., as described herein); a PKC inhibitor (e.g., LXS196 or sotrastaurin); an antibody-based therapeutic (e.g., an anti-PD-1 or anti-PDL1 antibody such as atezolizumab, pembrolizumab, nivolumab, or spartalizumab; an anti-CD20 antibody such as obinutuzumab or rituximab; or an anti-DR5 antibody such as drozitumab); a proteasome inhibitor (e.g., bortezomib, carfilzomib, ixazomib, or MG-132); an HDAC inhibitor (e.g., SAHA or VPA); an antibiotic (e.g., actinomycin D); a zinc-containing therapeutic (e.g., zinc or ZMC1); an HSP inhibitor (e.g., geldanamycin); an ATPase inhibitor (e.g., archazolid); a mitotic inhibitor (e.g., paclitaxel or vincristine); metformin; methotrexate; tanshinone IIA; and/or P5091.

In some embodiments, an anti-cancer therapy of the disclosure comprises a tyrosine kinase inhibitor, e.g., alone or in combination with an ALK-targeted therapy. In some embodiments, the tyrosine kinase inhibitor is an ALK-targeted anti-cancer therapy or treatment. In some embodiments, the tyrosine kinase inhibitor is (a) a small molecule that inhibits one or more enzymatic activities of a tyrosine kinase, (b) an antibody that inhibits one or more activities of a tyrosine kinase (e.g., by binding to and inhibiting one or more activities of the tyrosine kinase, binding to and inhibiting expression, such as cell surface expression, of the tyrosine kinase, and/or binding to and inhibiting one or more activities of a cell expressing the tyrosine kinase, such as by inducing antibody-dependent cellular cytotoxicity, ADCC, or phagocytosis, ADCP), or (c) a nucleic acid that inhibits expression of a tyrosine kinase (e.g., an antisense oligonucleotide, miRNA, siRNA, morpholino, CRISPR-based therapeutic, and the like). In some embodiments, the tyrosine kinase inhibitor is a small molecule inhibitor of a tyrosine kinase (e.g., a competitive or non-competitive inhibitor). Non-limiting examples of tyrosine kinase inhibitors include imatinib, crenolanib, linifanib, ninetedanib, axitinib, dasatinib, imetelstat, midostaurin, pazopanib, sorafenib, sunitinb, motesanib, masitinib, vatalanib, cabozanitinib, tivozanib, OSI-930, Ki8751, telatinib, dovitinib, tyrphostin AG 1296, and amuvatinib, as well as pharmaceutically acceptable salts thereof.

In some embodiments, an anti-cancer therapy of the disclosure comprises a mitogen-activated protein kinase (MEK) inhibitor, e.g., alone or in combination with an ALK-targeted therapy. In some embodiments, the MEK inhibitor inhibits one or more activities of MEK1 and/or MEK2. In some embodiments, the anti-cancer therapy/MEK inhibitor is (a) a small molecule that inhibits one or more enzymatic activities of MEK, (b) an antibody that inhibits one or more activities of MEK (e.g., by binding to and inhibiting one or more activities of MEK, binding to and inhibiting expression of MEK, and/or binding to and inhibiting one or more activities of a cell expressing MEK, such as by inducing antibody-dependent cellular cytotoxicity, ADCC, or phagocytosis, ADCP), or (c) a nucleic acid that inhibits expression of MEK (e.g., an antisense oligonucleotide, miRNA, siRNA, morpholino, CRISPR-based therapeutic, and the like). In some embodiments, the MEK inhibitor is a small molecule inhibitor of MEK (e.g., a competitive or non-competitive inhibitor). Non-limiting examples of MEK inhibitors include trametinib, cobimetinib, binimetinib, CI-1040, PD0325901, selumetinib, AZD8330, TAK-733, GDC-0623, refametinib, pimasertib, R04987655, R05126766, WX-544, and HL-085, as well as pharmaceutically acceptable salts thereof. In some embodiments, the anti-cancer therapy inhibits one or more activities of the Raf/MEK/ERK pathway, including inhibitors of Raf, MEK, and/or ERK.

In some embodiments, an anti-cancer therapy of the disclosure comprises a mammalian target of rapamycin (mTOR) inhibitor, e.g., alone or in combination with an ALK-targeted therapy. In some embodiments, the mTOR inhibitor is (a) a small molecule that inhibits one or more enzymatic activities of mTOR, (b) an antibody that inhibits one or more activities of mTOR (e.g., by binding to and inhibiting one or more activities of mTOR, binding to and inhibiting expression of mTOR, and/or binding to and inhibiting one or more activities of a cell expressing mTOR, such as by inducing antibody-dependent cellular cytotoxicity, ADCC, or phagocytosis, ADCP), or (c) a nucleic acid that inhibits expression of mTOR (e.g., an antisense oligonucleotide, miRNA, siRNA, morpholino, CRISPR-based therapeutic, and the like). In some embodiments, the mTOR inhibitor is a small molecule inhibitor of mTOR (e.g., a competitive inhibitor, such as an ATP-competitive inhibitor, or a non-competitive inhibitor, such as a rapamycin analog). Non-limiting examples of mTOR inhibitors include temsirolimus, everolimus, ridaforolimus, dactolisib, GSK2126458, XL765, AZD8055, AZD2014, MLN128, PP242, NVP-BEZ235, LY3023414, PQR309, PKI587, and OSI027, as well as pharmaceutically acceptable salts thereof. In some embodiments, the anti-cancer therapy inhibits one or more activities of the Akt/mTOR pathway, including inhibitors of Akt and/or mTOR.

In some embodiments, an anti-cancer therapy of the disclosure comprises a PI3K inhibitor or Akt inhibitor, e.g., alone or in combination with an ALK-targeted therapy. In some embodiments, the PI3K inhibitor inhibits one or more activities of PI3K. In some embodiments, the anti-cancer therapy/PI3K inhibitor is (a) a small molecule that inhibits one or more enzymatic activities of PI3K, (b) an antibody that inhibits one or more activities of PI3K (e.g., by binding to and inhibiting one or more activities of PI3K, binding to and inhibiting expression of PI3K, and/or binding to and inhibiting one or more activities of a cell expressing PI3K, such as by inducing antibody-dependent cellular cytotoxicity, ADCC, or phagocytosis, ADCP), or (c) a nucleic acid that inhibits expression of PI3K (e.g., an antisense oligonucleotide, miRNA, siRNA, morpholino, CRISPR-based therapeutic, and the like). In some embodiments, the PI3K inhibitor is a small molecule inhibitor of PI3K (e.g., a competitive or non-competitive inhibitor). Non-limiting examples of PI3K inhibitors include GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDCO0032, IPI145, INK1117 (MLN1117), SAR260301, KIN-193 (AZD6482), duvelisib, GS-9820, GSK2636771, GDC-0980, AMG319, pazobanib, and alpelisib (BYL719, Piqray), as well as pharmaceutically acceptable salts thereof. In some embodiments, the AKT inhibitor inhibits one or more activities of AKT (e.g., AKT1). In some embodiments, the AKT inhibitor is (a) a small molecule that inhibits one or more enzymatic activities of AKT1, (b) an antibody that inhibits one or more activities of AKT1 (e.g., by binding to and inhibiting one or more activities of AKT1, binding to and inhibiting expression of AKT1, and/or binding to and inhibiting one or more activities of a cell expressing AKT1, such as by inducing antibody-dependent cellular cytotoxicity, ADCC, or phagocytosis, ADCP), or (c) a nucleic acid that inhibits expression of AKT1 (e.g., an antisense oligonucleotide, miRNA, siRNA, morpholino, CRISPR-based therapeutic, and the like). In some embodiments, the AKT1 inhibitor is a small molecule inhibitor of AKT1 (e.g., a competitive or non-competitive inhibitor). Non-limiting examples of AKT1 inhibitors include GSK690693, GSK2141795 (uprosertib), GSK2110183 (afuresertib), AZD5363, GDC-0068 (ipatasertib), AT7867, CCT128930, MK-2206, BAY 1125976, AKT1 and AKT2-IN-1, perifosine, and VIII, as well as pharmaceutically acceptable salts thereof. In some embodiments, the AKT1 inhibitor is a pan-Akt inhibitor.

In some embodiments, an anti-cancer therapy of the disclosure comprises a hedgehog (Hh) inhibitor, e.g., alone or in combination with an ALK-targeted therapy. In some embodiments, the Hh inhibitor is (a) a small molecule that inhibits one or more enzymatic activities of Hh, (b) an antibody that inhibits one or more activities of Hh (e.g., by binding to and inhibiting one or more activities of Hh, binding to and inhibiting expression of Hh, and/or binding to and inhibiting one or more activities of a cell expressing Hh, such as by inducing antibody-dependent cellular cytotoxicity, ADCC, or phagocytosis, ADCP), or (c) a nucleic acid that inhibits expression of Hh (e.g., an antisense oligonucleotide, miRNA, siRNA, morpholino, CRISPR-based therapeutic, and the like). In some embodiments, the Hh inhibitor is a small molecule inhibitor of Hh (e.g., a competitive or non-competitive inhibitor). Non-limiting examples of Hh inhibitors include sonidegib, vismodegib, erismodegib, saridegib, BMS833923, PF-04449913, and LY2940680, as well as pharmaceutically acceptable salts thereof.

In some embodiments, an anti-cancer therapy of the disclosure comprises a heat shock protein (HSP) inhibitor, a MYC inhibitor, an HDAC inhibitor, an immunotherapy, a neoantigen, a vaccine, or a cellular therapy, e.g., alone or in combination with an ALK-targeted therapy.

In some embodiments, the anti-cancer therapy comprises one or more of an immune checkpoint inhibitor, a chemotherapy, a VEGF inhibitor, an Integrin β3 inhibitor, a statin, an EGFR inhibitor, an mTOR inhibitor, a PI3K inhibitor, a MAPK inhibitor, or a CDK4/6 inhibitor, e.g., alone or in combination with an ALK-targeted therapy.

In some embodiments, the anti-cancer therapy comprises a kinase inhibitor, e.g., alone or in combination with an ALK-targeted therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the kinase inhibitor is an ALK kinase inhibitor, e.g., as described herein and/or in examples 3-39 of WO2005016894, which is incorporated herein by reference.

In some embodiments, the anti-cancer therapy comprises a heat shock protein (HSP) inhibitor, e.g., alone or in combination with an ALK-targeted therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor, e.g., alone or in combination with an ALK-targeted therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor, e.g., alone or in combination with an ALK-targeted therapy. In some embodiments, the HDAC inhibitor is belinostat (PXD101, e.g., Beleodaq®), SAHA (vorinostat, suberoylanilide hydroxamine, e.g., Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor, e.g., alone or in combination with an ALK-targeted therapy. In some embodiments, the VEGF inhibitor is Bevacizumab (e.g., Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin 03 inhibitor, e.g., alone or in combination with an ALK-targeted therapy. In some embodiments, the integrin 03 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor, e.g., alone or in combination with an ALK-targeted therapy. In some embodiments, the αIIbβ3 integrin inhibitor is abciximab, eptifibatide (e.g., Integrilin®), or tirofiban (e.g., Aggrastat®).

In some embodiments, the anti-cancer therapy comprises an mTOR inhibitor, e.g., alone or in combination with an ALK-targeted therapy. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a statin or a statin-based agent, e.g., alone or in combination with an ALK-targeted therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises a MAPK inhibitor, e.g., alone or in combination with an ALK-targeted therapy. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises an EGFR inhibitor, e.g., alone or in combination with an ALK-targeted therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (ACO0010), rociletinib (CO-1686), BMS-690514, YH5448, PF-06747775, ASP8273, PF299804, AP26113, necitumumab (e.g., Portrazza®), or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., alone or in combination with an ALK-targeted therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen corresponding to a fusion nucleic acid molecule or polypeptide of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine, e.g., alone or in combination with an ALK-targeted therapy. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21, 2013). In some embodiments, such cancer vaccines augment the anti-cancer response.

In some embodiments, the cancer vaccine comprises a polynucleotide that encodes a neoantigen, e.g., a neoantigen corresponding to an ALK fusion nucleic acid molecule or polypeptide of the disclosure. In some embodiments, the cancer vaccine comprises DNA that encodes a neoantigen, e.g., a neoantigen corresponding to an ALK fusion nucleic acid molecule or polypeptide of the disclosure. In some embodiments, the cancer vaccine comprises RNA that encodes a neoantigen, e.g., a neoantigen corresponding to an ALK fusion nucleic acid molecule or polypeptide of the disclosure. In some embodiments, the cancer vaccine comprises a polynucleotide that encodes a neoantigen, e.g., a neoantigen corresponding to an ALK fusion nucleic acid molecule or polypeptide of the disclosure. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (e.g., Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (e.g., Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (e.g., Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen corresponding to an ALK fusion nucleic acid molecule or polypeptide of the disclosure.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen corresponding to a fusion nucleic acid molecule or polypeptide of the disclosure.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen corresponding to an ALK fusion nucleic acid molecule or polypeptide of the disclosure, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., a neoantigen corresponding to an ALK fusion nucleic acid molecule or polypeptide of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (e.g., PROLEUKIN®; interleukin-2), interferon alfa-2a (e.g., ROFERON®-A), interferon alfa-2b (e.g., INTRON®-A), and peginterferon alfa-2b (e.g., PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor, e.g., alone or in combination with an ALK-targeted therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSFIR, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCN1 (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No.Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is one or more of MDX-1 106 (nivolumab), MK-3475 (pembrolizumab, e.g., Keytruda®), MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (e.g., LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, e.g., Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No.Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1 105, MED14736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (IBI310, BMS-734016, MDX010, MDX-CTLA4, MED14736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, e.g., Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine, e.g., alone or in combination with an ALK-targeted therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3a (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises an anti-cancer agent that inhibits expression of a nucleic acid that comprises or encodes an ALK fusion nucleic acid molecule of the disclosure or a portion thereof, or an ALK fusion polypeptide of the disclosure, or a portion thereof. In some embodiments, the anti-cancer therapy comprises a nucleic acid molecule, such as a dsRNA, an siRNA, or an shRNA. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a nucleic acid that comprises or encodes an ALK fusion nucleic acid molecule of the disclosure or a portion thereof comprising a breakpoint. Methods for designing, optimizing, producing, and using dsRNAs, siRNAs, or shRNAs, are known in the art.

In some embodiments, the anti-cancer therapy comprises a chemotherapy, e.g., alone or in combination with an ALK-targeted therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′, 2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor, e.g., alone or in combination with an ALK-targeted therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR2, FGFR3, FGFR4, CSF1R, c-Met, ROSI, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, c-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent, e.g., alone or in combination with an ALK-targeted therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy, e.g., alone or in combination with an ALK-targeted therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer, e.g., alone or in combination with an ALK-targeted therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent, e.g., alone or in combination with an ALK-targeted therapy. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (e.g., Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (e.g., ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (e.g., Humira®), certolizumab pegol (e.g., Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, e.g., Ala-Cort®, Hydrocort Acetate®, hydrocortone phosphate Lanacort®, Solu-Cortef®), decadron (dexamethasone, dexamethasone acetate, dexamethasone sodium phosphate, e.g., Dexasone®, Diodex®, Hexadrol®, Maxidex®), methylprednisolone (6-methylprednisolone, methylprednisolone acetate, methylprednisolone sodium succinate, e.g., Duralone®, Medralone®, Medrol®, M-Prednisol®, Solu-Medrol®), prednisolone (e.g., Delta-Cortef®, ORAPRED®, Pediapred®, Prezone®), and prednisone (e.g., Deltasone®, Liquid Pred®, Meticorten®, Orasone®), and bisphosphonates (e.g., pamidronate (Aredia®), and zoledronic acid (e.g., Zometac®).

In some embodiments, the anti-cancer therapy comprises an anti-hormonal agent, e.g., alone or in combination with an ALK-targeted therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent, e.g., alone or in combination with an ALK-targeted therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (e.g., GEMZAR®), 5-fluorouracil (5-FU), capecitabine (e.g., XELODA™), 6-mercaptopurine, methotrexate, 6-thioguanine, pemetrexed, raltitrexed, arabinosylcytosine ARA-C cytarabine (e.g., CYTOSAR-U®), dacarbazine (DTIC-DOMED), azocytosine, deoxycytosine, pyridmidene, fludarabine (e.g., FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent, e.g., alone or in combination with an ALK-targeted therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein (e.g., an ALK-targeted therapy, and/or any other anti-cancer therapy provided herein), and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, an anti-cancer therapy of the disclosure (e.g., an ALK-targeted therapy) is administered as a monotherapy. In some embodiments, the anti-cancer therapy is administered in combination with one or more additional anti-cancer therapies or treatments, e.g., as described herein. In some embodiments, the one or more additional anti-cancer therapies or treatments include one or more anti-cancer therapies described herein. In some embodiments, the methods of the present disclosure comprise administration of any combination of any of the anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy. In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

### D. Reporting

In some embodiments, the methods provided herein comprise generating a report, and/or providing a report to party.

In some embodiments, a report according to the present disclosure comprises information about one or more of: an ALK fusion nucleic acid molecule or polypeptide of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule or polypeptide described herein); a cancer of the disclosure, e.g., comprising an ALK fusion nucleic acid molecule or polypeptide of the disclosure; or a treatment, a therapy, or one or more treatment options for an individual having a cancer, such as a cancer of the disclosure (e.g., comprising an ALK fusion nucleic acid molecule or polypeptide described herein).

In some embodiments, a report according to the present disclosure comprises information about the presence or absence of an ALK fusion nucleic acid molecule or polypeptide of the disclosure in a sample obtained from an individual, such as an individual having a cancer, e.g., a cancer provided herein. In one embodiment, a report according to the present disclosure indicates that an ALK fusion nucleic acid molecule or polypeptide of the disclosure is present in a sample obtained from the individual. In one embodiment, a report according to the present disclosure indicates that an ALK fusion nucleic acid molecule or polypeptide of the disclosure is not present in a sample obtained from the individual. In one embodiment, a report according to the present disclosure indicates that an ALK fusion nucleic acid molecule or polypeptide of the disclosure has been detected in a sample obtained from the individual. In one embodiment, a report according to the present disclosure indicates that an ALK fusion nucleic acid molecule or polypeptide of the disclosure has not been detected in a sample obtained from the individual. In some embodiments, the report comprises an identifier for the individual from which the sample was obtained.

In some embodiments, the report includes information on the role of an ALK fusion nucleic acid molecule or polypeptide of the disclosure in disease, such as in cancer. Such information can include one or more of: information on prognosis of a cancer, such as a cancer provided herein, e.g., comprising an ALK fusion nucleic acid molecule or polypeptide described herein; information on resistance of a cancer, such as a cancer provided herein, e.g., comprising an ALK fusion nucleic acid molecule or polypeptide described herein, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., comprising an ALK fusion nucleic acid molecule or polypeptide described herein and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting an ALK fusion nucleic acid molecule or polypeptide of the disclosure in the sample, or acquiring knowledge of the presence of the ALK fusion nucleic acid molecule or polypeptide of the disclosure in the sample; and generating a report. In some embodiments, a report generated according to the methods provided herein comprises one or more of: information about the presence or absence of an ALK fusion nucleic acid molecule or polypeptide of the disclosure in the sample; an identifier for the individual from which the sample was obtained; information on the role of the ALK fusion nucleic acid molecule or polypeptide of the disclosure, or its wild type counterparts, in disease (e.g., such as in cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient having, suspected of having, or being tested for a cancer, such as a cancer provided herein, e.g., comprising an ALK fusion nucleic acid molecule or polypeptide of the disclosure), or to an individual or entity other than the individual or patient, such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from the individual. In some embodiments, the report is provided or delivered to an individual or entity within any of about 1 day or more, about 7 days or more, about 14 days or more, about 21 days or more, about 30 days or more, about 45 days or more, or about 60 days or more from detecting an ALK fusion nucleic acid molecule or polypeptide of the disclosure in a sample obtained from the individual. In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of an ALK fusion nucleic acid molecule or polypeptide of the disclosure in a sample obtained from the individual.

### E. Software, Systems, and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

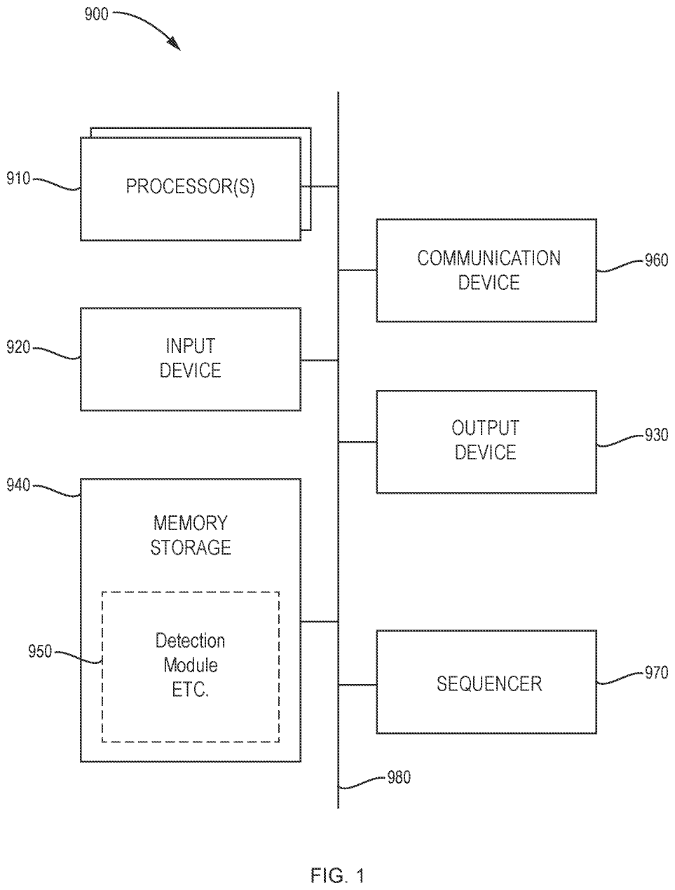

FIG. 1 illustrates an example of a computing device or system in accordance with one embodiment. Device 900 can be a host computer connected to a network. Device 900 can be a client computer or a server. As shown in FIG. 1, device 900 can be any suitable type of microprocessor-based device, such as a personal computer, workstation, server or handheld computing device (portable electronic device) such as a phone or tablet. The device can include, for example, one or more processor(s) 910, input devices 920, output devices 930, memory or storage devices 940, communication devices 960, and nucleic acid sequencers 970. Software 950 residing in memory or storage device 940 may comprise, e.g., an operating system as well as software for executing the methods described herein, e.g., for detecting an ALK fusion nucleic acid molecule of the disclosure. Input device 920 and output device 930 can generally correspond to those described herein, and can either be connectable or integrated with the computer.

Input device 920 can be any suitable device that provides input, such as a touch screen, keyboard or keypad, mouse, or voice-recognition device. Output device 930 can be any suitable device that provides output, such as a touch screen, haptics device, or speaker.

Storage 940 can be any suitable device that provides storage (e.g., an electrical, magnetic or optical memory including a RAM (volatile and non-volatile), cache, hard drive, or removable storage disk). Communication device 960 can include any suitable device capable of transmitting and receiving signals over a network, such as a network interface chip or device. The components of the computer can be connected in any suitable manner, such as via a wired media (e.g., a physical system bus 980, Ethernet connection, or any other wire transfer technology) or wirelessly (e.g., Bluetooth®, Wi-Fi®, or any other wireless technology).

Software module 950, which can be stored as executable instructions in storage 940 and executed by processor(s) 910, can include, for example, an operating system and/or the processes that embody the functionality of the methods of the present disclosure, e.g., for detecting an ALK fusion nucleic acid molecule of the disclosure (e.g., as embodied in the devices as described herein).

Software module 950 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 940, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Software module 950 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

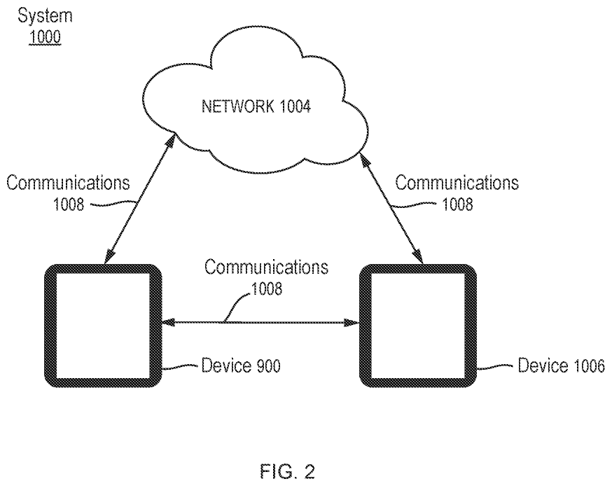

Device 900 may be connected to a network (e.g., network 1004, as shown in FIG. 2 and described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 900 can be implemented using any operating system, e.g., an operating system suitable for operating on the network. Software module 950 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, the operating system is executed by one or more processors, e.g., processor(s) 910.

Device 900 can further include a sequencer 970, which can be any suitable nucleic acid sequencing instrument. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

FIG. 2 illustrates an example of a computing system in accordance with one embodiment. In computing system 1000, device 900 (e.g., as described above and illustrated in FIG. 1) is connected to network 1004, which is also connected to device 1006. In some embodiments, device 1006 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system.

Devices 900 and 1006 may communicate, e.g., using suitable communication interfaces via network 1004, such as a Local Area Network (LAN), Virtual Private Network (VPN), or the Internet. In some embodiments, network 1004 can be, for example, the Internet, an intranet, a virtual private network, a cloud network, a wired network, or a wireless network. Devices 900 and 1006 may communicate, in part or in whole, via wireless or hardwired communications, such as Ethernet, IEEE 802.11b wireless, or the like. Additionally, devices 900 and 1006 may communicate, e.g., using suitable communication interfaces, via a second network, such as a mobile/cellular network. Communication between devices 900 and 1006 may further include or communicate with various servers such as a mail server, mobile server, media server, telephone server, and the like. In some embodiments, devices 900 and 1006 can communicate directly (instead of, or in addition to, communicating via network 1004), e.g., via wireless or hardwired communications, such as Ethernet, IEEE 802.11b wireless, or the like. In some embodiments, devices 900 and 1006 communicate via communications 1008, which can be a direct connection or can occur via a network (e.g., network 1004).

One or all of devices 900 and 1006 generally include logic (e.g., http web server logic) or are programmed to format data, accessed from local or remote databases or other sources of data and content, for providing and/or receiving information via network 1004 according to various examples described herein.

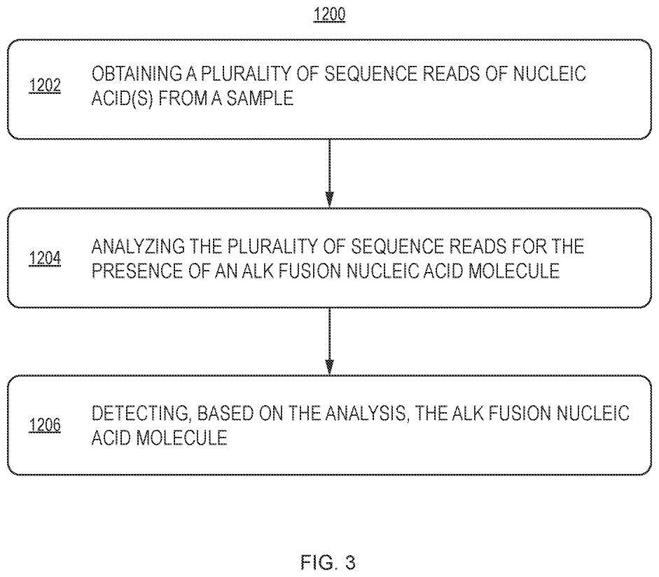

FIG. 3 illustrates an exemplary process 1200 for detecting an ALK fusion nucleic acid molecule of the disclosure in a sample, in accordance with some embodiments of the present disclosure. Process 1200 is performed, for example, using one or more electronic devices implementing a software program. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In some embodiments, the executed steps can be executed across many systems, e.g., in a cloud environment. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At block 1202, a plurality of sequence reads of one or more nucleic acid molecules is obtained, wherein the one or more nucleic acid molecules are derived from a sample obtained from an individual, e.g., as described herein. In some embodiments, the sample is obtained from an individual having, suspected of having, or being tested for a cancer, such as a cancer described herein. In some embodiments, the sequence reads are obtained using a sequencer, e.g., as described herein or otherwise known in the art. In some embodiments, the nucleic acid molecules comprise one or more nucleic acid molecules corresponding to: an ALK fusion nucleic acid molecule of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein); or a gene involved in an ALK fusion nucleic acid molecule of the disclosure; or fragments thereof. Optionally, prior to obtaining the sequence reads, the sample is purified, enriched (e.g., for nucleic acid(s) corresponding to: an ALK fusion nucleic acid molecule of the disclosure; or a gene involved in an ALK fusion nucleic acid molecule of the disclosure; or fragments thereof), and/or subjected to PCR amplification. At block 1204, an exemplary system (e.g., one or more electronic devices) analyzes the plurality of sequence reads for the presence of an ALK fusion nucleic acid molecule of the disclosure, or a fragment thereof. At block 1206, the system detects (e.g., based on the analysis) an ALK fusion nucleic acid molecule of the disclosure, or a fragment thereof, in the sample.

In some embodiments of any of the methods, systems, devices, non-transitory computer readable storage media, or processes of the disclosure, the ALK fusion nucleic acid molecule is any of the ALK fusion nucleic acid molecules provides herein. In some embodiments of any of the methods, systems, devices, non-transitory computer readable storage media, or processes of the disclosure, the ALK fusion nucleic acid molecule comprises a fusion between an ALK gene, or a portion thereof, and a gene listed in Table 1, or a portion thereof. In some embodiments of any of the methods, systems, devices, non-transitory computer readable storage media, or processes of the disclosure, the ALK fusion nucleic acid molecule is an ALK fusion nucleic acid molecule listed in Table 2, and wherein the order of the genes in the fusion in the 5′ to 3′ direction is as listed in Table 2. In some embodiments of any of the methods, systems, devices, non-transitory computer readable storage media, or processes of the disclosure, the ALK fusion nucleic acid molecule is an ALK fusion nucleic acid molecule listed in Table 3, and wherein the ALK fusion nucleic acid molecule comprises or results from a corresponding 5′ breakpoint and/or 3′ breakpoint within the exons or introns as listed in Table 3. In some embodiments of any of the methods, systems, devices, non-transitory computer readable storage media, or processes of the disclosure, the ALK fusion nucleic acid molecule is an ALK fusion nucleic acid molecule listed in Table 4, and wherein the ALK fusion nucleic acid molecule comprises or results from a corresponding 5′ breakpoint within the chromosomal coordinates as listed in Table 4, and/or a corresponding 3′ breakpoint within the chromosomal coordinates as listed in Table 4. In some embodiments of any of the methods, systems, devices, non-transitory computer readable storage media, or processes of the disclosure, the ALK fusion nucleic acid molecule is an ALK fusion nucleic acid molecule listed in Table 5, and wherein the ALK fusion nucleic acid molecule comprises or results from a fusion of a corresponding 5′ exon as listed in Table 5, or a portion thereof, fused to a corresponding 3′ exon as listed in Table 5, or a portion thereof. In some embodiments of any of the methods, systems, devices, non-transitory computer readable storage media, or processes of the disclosure, the ALK fusion nucleic acid molecule is an ALK fusion nucleic acid molecule listed in Table 6, and wherein the ALK fusion nucleic acid molecule comprises, in the 5′ to 3′ direction, the corresponding exons or portions thereof as listed in Table 6. In some embodiments of any of the methods, systems, devices, non-transitory computer readable storage media, or processes of the disclosure, the ALK fusion nucleic acid molecule is an ALK fusion nucleic acid molecule listed in Table 7, and wherein the ALK fusion nucleic acid molecule comprises a corresponding nucleotide sequence as listed in Table 7, or a nucleotide sequence with at least about 70% homology thereto. In some embodiments of any of the methods, systems, devices, non-transitory computer readable storage media, or processes of the disclosure, the ALK fusion nucleic acid molecule encodes an ALK fusion polypeptide. In some embodiments of any of the methods, systems, devices, non-transitory computer readable storage media, or processes of the disclosure, the ALK fusion nucleic acid molecule comprises a nucleotide sequence encoding an ALK fusion polypeptide that comprises an amino acid sequence as listed in Table 8, or an amino acid sequence with at least about 70% homology thereto. In some embodiments of any of the methods, systems, devices, non-transitory computer readable storage media, or processes of the disclosure, the encoded ALK fusion polypeptide comprises an ALK kinase domain, or a fragment of an ALK kinase domain, having ALK kinase activity. In some embodiments of any of the methods, systems, devices, non-transitory computer readable storage media, or processes of the disclosure, the encoded ALK fusion polypeptide has ALK kinase activity. In some embodiments of any of the methods, systems, devices, non-transitory computer readable storage media, or processes of the disclosure, the encoded ALK fusion polypeptide has a constitutive ALK kinase activity. In some embodiments of any of the methods, systems, devices, non-transitory computer readable storage media, or processes of the disclosure, the encoded ALK fusion polypeptide is oncogenic. In some embodiments of any of the methods, systems, devices, non-transitory computer readable storage media, or processes of the disclosure, the encoded ALK fusion polypeptide promotes cancer cell survival, angiogenesis, cancer cell proliferation, and any combination thereof. In some embodiments of any of the methods, systems, devices, non-transitory computer readable storage media, or processes of the disclosure, the encoded ALK fusion polypeptide is capable of dimerizing with an ALK polypeptide or with another ALK fusion polypeptide in a cancer cell. In some embodiments of any of the methods, systems, devices, non-transitory computer readable storage media, or processes of the disclosure, the encoded ALK fusion polypeptide is an ALK fusion polypeptide listed in Table 8, and wherein the encoded ALK fusion polypeptide comprises a corresponding amino acid sequence as listed in Table 8, or an amino acid sequence with at least about 70% homology thereto.

In some embodiments of any of the methods, systems, devices, non-transitory computer readable storage media, or processes of the disclosure, the cancer is a carcinoma, a sarcoma, a lymphoma, a leukemia, a myeloma, a germ cell cancer, or a blastoma. In some embodiments of any of the methods, systems, devices, non-transitory computer readable storage media, or processes of the disclosure, the cancer is a solid tumor or a hematologic malignancy. In some embodiments of any of the methods, systems, devices, non-transitory computer readable storage media, or processes of the disclosure, the cancer is a lymphoma. In some embodiments of any of the methods, systems, devices, non-transitory computer readable storage media, or processes of the disclosure, the cancer is a non-small cell lung carcinoma, a leiomyosarcoma, a thyroid carcinoma, a colorectal cancer, a pancreatic cancer, or a malignant peritoneal mesothelioma. In some embodiments of any of the methods, systems, devices, non-transitory computer readable storage media, or processes of the disclosure, the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor. In some embodiments of any of the methods, systems, devices, non-transitory computer readable storage media, or processes of the disclosure, the cancer is an anus squamous cell carcinoma, brain glioblastoma (GBM), breast cancer, breast carcinoma, breast invasive ductal carcinoma (IDC), colon adenocarcinoma (CRC), esophagus adenocarcinoma, fallopian tube serous carcinoma, gallbladder adenocarcinoma, gallbladder carcinoma, gastroesophageal junction adenocarcinoma, lung adenocarcinoma, lung non-small cell lung carcinoma, lung non-small cell lung carcinoma, lymph node Castleman's disease, lymph node lymphoma T-cell, ovary clear cell carcinoma, ovary endometrioid adenocarcinoma, ovary epithelial carcinoma, ovary high grade serous carcinoma, ovary serous carcinoma, pancreas cancer, pancreas ductal adenocarcinoma, pediatric bone osteosarcoma, bone osteosarcoma, pediatric skin melanoma, skin melanoma, pediatric soft tissue sarcoma, soft tissue sarcoma, pediatric soft tissue sarcoma undifferentiated, soft tissue sarcoma undifferentiated, peritoneum serous carcinoma, prostate acinar adenocarcinoma, small intestine adenocarcinoma, soft tissue leiomyosarcoma, soft tissue liposarcoma, thyroid papillary carcinoma, unknown primary adenocarcinoma, unknown primary carcinoma, unknown primary malignant neoplasm, unknown primary myoepithelial carcinoma, uterus carcinosarcoma, uterus endometrial adenocarcinoma endometrioid, uterus leiomyosarcoma, or vulva squamous cell carcinoma (SCC). In some embodiments of any of the methods, systems, devices, non-transitory computer readable storage media, or processes of the disclosure, the ALK fusion nucleic acid molecule is an ALK fusion nucleic acid molecule listed in Table 9, or the encoded ALK fusion polypeptide is an ALK fusion polypeptide listed in Table 9, and wherein the cancer is the corresponding cancer as listed in Table 9.

In some embodiments of any of the methods, systems, devices, non-transitory computer readable storage media, or processes of the disclosure, the plurality of sequence reads is obtained by sequencing nucleic acids obtained from any of the samples described herein, e.g., tissue and/or liquid biopsies, etc. In some embodiments, the sample is obtained from the cancer. In some embodiments, the sample comprises a tissue biopsy sample, a liquid biopsy sample, or a normal control. In some embodiments, the sample is from a tumor biopsy, tumor specimen, or circulating tumor cell. In some embodiments, the sample is a liquid biopsy sample and comprises blood, plasma, cerebrospinal fluid, sputum, stool, urine, or saliva. In some embodiments, the sample comprises cells and/or nucleic acids from the cancer. In some embodiments, the sample comprises mRNA, DNA, circulating tumor DNA (ctDNA), cell-free DNA, or cell-free RNA from the cancer. In some embodiments, the sample is a liquid biopsy sample and comprises circulating tumor cells (CTCs). In some embodiments, the sample is a liquid biopsy sample and comprises cell-free DNA (cfDNA), circulating tumor DNA (ctDNA), or any combination thereof.

In some embodiments of any of the methods, systems, devices, non-transitory computer readable storage media, or processes of the disclosure, the plurality of sequence reads is obtained by sequencing. In some embodiments, the sequencing comprises use of a massively parallel sequencing (MPS) technique, whole genome sequencing (WGS), whole exome sequencing, targeted sequencing, direct sequencing, or a Sanger sequencing technique. In some embodiments, the massively parallel sequencing technique comprises next generation sequencing (NGS).

In some embodiments of any of the methods, systems, devices, non-transitory computer readable storage media, or processes of the disclosure, the disclosed methods for determining the presence or absence of a fusion nucleic acid molecule of the disclosure may be implemented as part of a genomic profiling process that comprises identification of the presence of variant sequences at one or more gene loci in a sample derived from an individual as part of detecting, monitoring, predicting a risk factor, or selecting a treatment for a particular disease, e.g., cancer. In some instances, the variant panel selected for genomic profiling may comprise the detection of variant sequences at a selected set of gene loci. In some instances, the variant panel selected for genomic profiling may comprise detection of variant sequences at a number of gene loci through comprehensive genomic profiling (CGP), a next-generation sequencing (NGS) approach used to assess hundreds of genes (including relevant cancer biomarkers) in a single assay. Inclusion of the disclosed methods for determining the presence or absence of an ALK fusion nucleic acid molecule of the disclosure as part of a genomic profiling process can improve the validity of, e.g., disease detection calls, made on the basis of the molecular profile by, for example, independently confirming the presence of the ALK fusion nucleic acid molecule of the disclosure in a given patient sample.

In some instances, a molecular profile may comprise information on the presence of genes (or variant sequences thereof), copy number variations, epigenetic traits, proteins (or modifications thereof), and/or other biomarkers in an individual's genome and/or proteome, as well as information on the individual's corresponding phenotypic traits and the interaction between genetic or genomic traits, phenotypic traits, and environmental factors.

In some instances, a molecular profile for the individual may comprise results from a comprehensive genomic profiling (CGP) test, a nucleic acid sequencing-based test, a gene expression profiling test, a cancer hotspot panel test, a DNA methylation test, a DNA fragmentation test, an RNA fragmentation test, or any combination thereof.

Accordingly, in some embodiments of any of the methods, systems, devices, non-transitory computer readable storage media, or processes of the disclosure, a molecular profile for the sample or for the individual is generated based, at least in part, on detecting an ALK fusion nucleic acid molecule of the disclosure, or a fragment thereof, in the sample. In some embodiments, the individual is administered a treatment based at least in part on the molecular profile, e.g., a treatment described herein such as an ALK-targeted therapy. In some embodiments, the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof. In some embodiments, the molecular profile further comprises results from a nucleic acid sequencing-based test. In some embodiments, the molecular profile further comprises/indicates/comprises information on presence or absence of mutations in one or more additional genes, e.g., a panel of known/suspected oncogenes and/or tumor suppressors. In some embodiments, the molecular profile is obtained from a genomic profiling assay (such as a cancer- or tumor-related genomic profiling assay), e.g., as obtained using any of the sequencing methodologies described herein. In some embodiments, the molecular profile includes information from whole-genome or whole-exome sequencing. In some embodiments, the molecular profile includes information from targeted sequencing. In some embodiments, the molecular profile includes information from NGS. In some embodiments, the molecular profile comprises/indicates/comprises information on presence or absence of mutations such as short variant alterations (e.g., a base substitution, insertion, or deletion), copy-number alterations (e.g., an amplification or a homozygous deletion), and/or rearrangements (e.g., a gene fusion or other genomic or chromosomal rearrangement) of one or more genes, e.g., a panel of known/suspected oncogenes and/or tumor suppressors.

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### IV. Articles of Manufacture or Kits

Provided herein are kits or articles of manufacture comprising one or more reagents for detecting an ALK fusion nucleic acid molecule or polypeptide of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule or polypeptide described herein) in a sample.

In some embodiments, the kits or articles of manufacture comprise one or more probes of the disclosure for detecting an ALK fusion nucleic acid molecule of the disclosure in a sample, e.g., according to any detection method known in the art or described herein. In some embodiments, the kits or articles of manufacture comprise one or more baits (e.g., one or more bait molecules) of the disclosure for detecting an ALK fusion nucleic acid molecule of the disclosure in a sample, e.g., according to any detection method known in the art or described herein. In some embodiments, the kits or articles of manufacture comprise one or more oligonucleotides (e.g., one or more primers) of the disclosure for detecting an ALK fusion nucleic acid molecule of the disclosure in a sample, e.g., according to any detection method known in the art or described herein. In some embodiments of any of the kits or articles of manufacture provided herein, the kit or article of manufacture comprises a reagent (e.g., one or more oligonucleotides, primers, probes or baits of the present disclosure) for detecting a wild-type counterpart of an ALK fusion nucleic acid molecule of the disclosure (e.g., a wild type ALK gene, and/or a wild type fusion partner gene described herein and/or in Tables 1-7, and/or in the Examples herein). In some embodiments, one or more oligonucleotides, primers, probes or baits are capable of hybridizing to an ALK fusion nucleic acid molecule of the disclosure, or to a wild-type counterpart of the ALK fusion nucleic acid molecule (e.g., a wild type ALK gene, and/or a wild type fusion partner gene described herein and/or in Tables 1-7, and/or in the Examples herein). In some embodiments, the one or more oligonucleotides, primers, probes or baits of the present disclosure are capable of distinguishing an ALK fusion nucleic acid molecule of the disclosure from a wild-type counterpart of the ALK fusion nucleic acid molecule (e.g., a wild type ALK gene, and/or a wild type fusion partner gene described herein and/or in Tables 1-7, and/or in the Examples herein). In some embodiments, the kit is for use according to any method of detecting fusion nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting an ALK fusion nucleic acid molecule of the disclosure, e.g., using one or more oligonucleotides, primers, probes or baits of the present disclosure.

In some embodiments, the kits or articles of manufacture comprise one or more antibodies or antibody fragments of the disclosure for detecting an ALK fusion polypeptide encoded of the disclosure, e.g., according to any detection method known in the art or described herein. In some embodiments, the kit or article of manufacture comprises a reagent (e.g., one or more antibodies of the present disclosure) for detecting the wild-type counterparts of an ALK fusion polypeptide provided herein (e.g., a wild type ALK polypeptide, and/or a wild type polypeptide encoded by a fusion partner gene described herein and/or in Tables 1-8, and/or in the Examples herein). In some embodiments, the kits or articles of manufacture comprise one or more antibodies of the present disclosure capable of binding to an ALK fusion polypeptide provided herein, or to wild-type counterparts of the ALK fusion polypeptide provided herein (e.g., a wild type ALK polypeptide, and/or a wild type polypeptide encoded by a fusion partner gene described herein and/or in Tables 1-8, and/or in the Examples herein). In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting an ALK fusion polypeptide of the disclosure, e.g., using one or more antibodies of the present disclosure.

Further provided herein are kits or articles of manufacture comprising an anti-cancer therapy, such as an anti-cancer therapy described herein, and a package insert comprising instructions for using the anti-cancer therapy in a method of treating or delaying progression of cancer, e.g., by administration to an individual from whom a sample comprising an ALK fusion nucleic acid molecule or polypeptide of the disclosure has been obtained. In some embodiments, the anti-cancer therapy is any of the anti-cancer therapies described herein for use in any of the methods for treating or delaying progression of cancer of the disclosure. In some embodiments, the anti-cancer therapy is an ALK-targeted therapy.

The kit or article of manufacture may include, for example, a container and a label or package insert on or associated with the container. Suitable containers include, for example, bottles, vials, syringes, and the like. The container may be formed from a variety of materials such as glass or plastic. The container holds or contains a composition comprising one or more reagents for detecting an ALK fusion nucleic acid molecule or polypeptide of the disclosure (e.g., one or more oligonucleotides, primers, probes, baits, antibodies or antibody fragments of the present disclosure) or one or more anti-cancer therapies of the disclosure. In some embodiments, the container holds or contains a composition comprising one or more anti-cancer therapies of the disclosure and may have a sterile access port (e.g., the container may be an intravenous solution bag or a vial having a stopper pierceable by a hypodermic injection needle).

The kit or article of manufacture may further include a second container comprising a diluent or buffer, e.g., a pharmaceutically-acceptable diluent buffer, such as bacteriostatic water for injection (BWFI), phosphate-buffered saline, Ringer's solution, and/or dextrose solution. The article of manufacture may further include other materials desirable from a commercial and user standpoint, including other buffers, diluents, filters, needles, and syringes.

The kit or article of manufacture of the present disclosure also includes information or instructions, for example in the form of a package insert, indicating that the one or more reagents and/or anti-cancer therapies are used for detecting an ALK fusion nucleic acid molecule or polypeptide of the disclosure, or for treating cancer, as described herein. The insert or label may take any form, such as paper or on electronic media such as a magnetically recorded medium (e.g., floppy disk), a CD-ROM, a Universal Serial Bus (USB) flash drive, and the like. The label or insert may also include other information concerning the pharmaceutical compositions and dosage forms in the kit or article of manufacture.

### V. Nucleic Acids, Vectors, Host Cells and Recombinant Cells

Provided herein are nucleic acids and vectors comprising or encoding an ALK fusion nucleic acid molecule of the disclosure (e.g., an ALK-ABCB11, ALK-ACTN4, ALK-AGAP1, ALK-APH1A, ALK-AZI2, ALK-BTBD9, ALK-C2orf73, ALK-CAPN14, ALK-CARMIL1, ALK-CASP8, ALK-CDC42BPA, ALK-CIB4, ALK-CNTNAP5, ALK-COL3A1, ALK-CPQ, ALK-CPSF7, ALK-CREBBP, ALK-CTBP1, ALK-CTNND1, ALK-CYP51A1, ALK-CYS1, ALK-EPHA2, ALK-FHOD3, ALK-FILIP1L, ALK-GMCL1, ALK-GPN1, ALK-GPR113, ALK-HADHA, ALK-HS1BP3, ALK-INTS9, ALK-ITGA6, ALK-KCTD18, ALK-KIF5C, ALK-KLC4, ALK-LINC00535, ALK-LRRFIP2, ALK-MAGOHB, ALK-MAMDC4, ALK-MANBA, ALK-MAP3K9, ALK-MED13L, ALK-METTL25, ALK-MTBP, ALK-MYH10, ALK-MYO5C, ALK-NFIA, ALK-NINJ2, ALK-OPRM1, ALK-OTX1, ALK-PAQR4, ALK-PDCD10, ALK-PDE3A, ALK-PELI1, ALK-PLEC, ALK-PTGER4, ALK-PTPRJ, ALK-QKI, ALK-RPS6KA5, ALK-SASH1, ALK-SEC16B, ALK-SKAP1, ALK-SLC25A13, ALK-SLC30A6, ALK-SNX17, ALK-SOX13, ALK-SRSF7, ALK-TANGO6, ALK-TG, ALK-TMCO3, ALK-TNS3, ALK-TRIM24, ALK-TTC28, ALK-UBE2L3, ALK-UBE3B, ALK-UTRN, ALK-VASP, ALK-WDR92, ALK-YPEL5, ALK-ZNF446, ALK-ZNF454, ALK-ZNF513, or ALK-ZSWIM2 fusion nucleic acid molecule described herein), or a bait, a probe, or an oligonucleotide described herein, or fragments thereof.

In some embodiments, a nucleic acid or vector provided herein comprises or encodes an ALK fusion nucleic acid molecule of the disclosure, or a nucleic acid molecule encoding an ALK fusion polypeptide described herein.

In some embodiments, a vector provided herein is a nucleic acid molecule capable of transporting another nucleic acid to which it has been linked (e.g., ALK fusion nucleic acid molecules, baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising ALK fusion nucleic acid molecules, baits, probes, or oligonucleotides described herein, or fragments thereof) are also contemplated herein, including, e.g., replication defective retroviruses, adenoviruses and adeno-associated viruses.

In some embodiments, a nucleic acid or vector provided herein comprises an ALK fusion nucleic acid molecule, a bait, a probe, or an oligonucleotide of the disclosure in a form suitable for expression thereof in a host cell. In some embodiments, the nucleic acid or vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., ALK fusion nucleic acid molecules, baits, probes, or oligonucleotides described herein, or fragments thereof). In some embodiments, a regulatory sequence directs tissue-specific expression of a nucleotide sequence (e.g., AKJ fusion nucleic acid molecules, baits, probes, or oligonucleotides described herein, or fragments thereof). In some embodiments, a regulatory sequence directs inducible expression of a nucleotide sequence (e.g., ALK fusion nucleic acid molecules, baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises or encodes an ALK fusion nucleic acid molecule, a bait, a probe, or an oligonucleotide of the disclosure in the sense or the anti-sense orientation. In some embodiments, a nucleic acid or vector (e.g., an expression vector) provided herein is introduced into host cells to thereby produce a polypeptide, e.g., an ALK fusion polypeptide described herein, or a fragment or mutant form thereof.

In some embodiments, the design of a nucleic acid or vector provided herein depends on such factors as the choice of the host cell to be transformed, the level of expression desired, and the like. In some embodiments, expression vectors are designed for the expression of the ALK fusion nucleic acid molecules, baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises or encodes an ALK fusion nucleic acid molecule described herein, wherein the nucleotide sequence of the ALK fusion nucleic acid molecule described herein has been altered (e.g., codon optimized) so that the individual codons for each encoded amino acid are those preferentially utilized in the host cell.

Also provided herein are host cells, e.g., comprising ALK fusion nucleic acid molecules, ALK fusion polypeptides, baits, probes, nucleic acids, vectors, or oligonucleotides of the disclosure. In some embodiments, a host cell (e.g., a recombinant host cell or recombinant cell) comprises a vector described herein (e.g., an expression vector described herein). In some embodiments, an ALK fusion nucleic acid molecule, bait, probe, nucleic acid, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

ALK fusion nucleic acid molecules, baits, probes, nucleic acids, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing an ALK fusion polypeptide of the disclosure, e.g., by culturing a host cell described herein (e.g., into which a recombinant expression vector encoding an ALK fusion polypeptide has been introduced) in a suitable medium such that the ALK fusion polypeptide is produced. In another embodiment, the method further includes isolating an ALK fusion polypeptide from the medium or the host cell.

### VI. Exemplary Embodiments

The following exemplary embodiments are representative of some aspects of the invention: Exemplary Embodiment 1: A method of identifying an individual having a cancer who may benefit from a treatment comprising an anaplastic lymphoma kinase (ALK)-targeted therapy, the method comprising detecting in a sample from the individual:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule,
wherein detection of the ALK fusion nucleic acid molecule or polypeptide in the sample identifies the individual as one who may benefit from the treatment comprising the ALK-targeted therapy.

Exemplary Embodiment 2: A method of selecting a therapy for an individual having a cancer, the method comprising detecting in a sample from the individual:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule,
wherein detection of the ALK fusion nucleic acid molecule or polypeptide in the sample identifies the individual as one who may benefit from a treatment comprising an ALK-targeted therapy.

Exemplary Embodiment 3: A method of identifying one or more treatment options for an individual having a cancer, the method comprising:
(a) detecting in a sample from the individual:
   (i) an ALK fusion nucleic acid molecule comprising a fusion between an ALK gene, or a portion thereof, and a gene listed in Table 1, or a portion thereof, or
   (ii) an ALK fusion polypeptide encoded by the ALK fusion nucleic acid molecule; and
(b) generating a report comprising one or more treatment options identified for the individual based at least in part on detection of the ALK fusion nucleic acid molecule or polypeptide in the sample, wherein the one or more treatment options comprise an ALK-targeted therapy.

Exemplary Embodiment 4: A method of identifying one or more treatment options for an individual having a cancer, the method comprising:
(a) acquiring knowledge of:
   (i) an ALK fusion nucleic acid molecule comprising a fusion between an ALK gene, or a portion thereof, and a gene listed in Table 1, or a portion thereof, or
   (ii) an ALK fusion polypeptide encoded by the ALK fusion nucleic acid molecule, in a sample from the individual; and
(b) generating a report comprising one or more treatment options identified for the individual based at least in part on said knowledge, wherein the one or more treatment options comprise an ALK-targeted therapy.

Exemplary Embodiment 5: A method of selecting a treatment for an individual having cancer, comprising acquiring knowledge of:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule, in a sample from an individual having a cancer,
wherein responsive to the acquisition of said knowledge: (i) the individual is classified as a candidate to receive a treatment comprising an ALK-targeted therapy; and/or (ii) the individual is identified as likely to respond to a treatment that comprises an ALK-targeted therapy.

Exemplary Embodiment 6: A method of predicting survival of an individual having a cancer, comprising acquiring knowledge of:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule, in a sample from the individual,
wherein responsive to the acquisition of said knowledge, the individual is predicted to have longer survival when treated with a treatment comprising an ALK-targeted therapy, as compared to survival of an individual whose cancer does not comprise an ALK fusion nucleic acid molecule or polypeptide.

Exemplary Embodiment 7: A method of predicting survival of an individual having a cancer treated with a treatment comprising an ALK-targeted therapy, the method comprising acquiring knowledge of:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule, in a sample from the individual,
wherein responsive to the acquisition of said knowledge, the individual is predicted to have longer survival when treated with a treatment comprising an ALK-targeted therapy, as compared to an individual whose cancer does not exhibit an ALK fusion nucleic acid molecule or polypeptide.

Exemplary Embodiment 8: A method of treating or delaying progression of cancer, comprising:
(a) acquiring knowledge of:
   (i) an ALK fusion nucleic acid molecule comprising a fusion between an ALK gene, or a portion thereof, and a gene listed in Table 1, or a portion thereof, or
   (ii) an ALK fusion polypeptide encoded by the ALK fusion nucleic acid molecule, in a sample from an individual having a cancer; and
(b) responsive to said knowledge, administering to the individual an effective amount of a treatment that comprises an ALK-targeted therapy.

Exemplary Embodiment 9: A method of treating or delaying progression of cancer, comprising administering to an individual having cancer an effective amount of a treatment that comprises an ALK-targeted therapy, wherein the ALK-targeted therapy is administered responsive to acquiring knowledge of:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule, in a sample from the individual.

Exemplary Embodiment 10: A method of monitoring, evaluating or screening an individual having a cancer, comprising acquiring knowledge of:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule, in a sample from the individual,
wherein responsive to the acquisition of said knowledge, the individual is predicted to have increased risk of cancer recurrence, aggressive cancer, anti-cancer therapy resistance, increased ALK expression, clinical benefit from an ALK-targeted therapy, or poor prognosis, as compared to an individual whose cancer does not comprise an ALK fusion nucleic acid molecule or polypeptide.

Exemplary Embodiment 11: The method of embodiment 10, wherein responsive to the acquisition of said knowledge, the individual is predicted to have resistance to a non-ALK-targeted anti-cancer therapy.

Exemplary Embodiment 12: A method of assessing an ALK fusion nucleic acid molecule or polypeptide in a cancer in an individual, the method comprising:
(a) detecting in a sample from the individual:
   (i) an ALK fusion nucleic acid molecule comprising a fusion between an ALK gene, or a portion thereof, and a gene listed in Table 1, or a portion thereof, or
   (ii) an ALK fusion polypeptide encoded by the ALK fusion nucleic acid molecule; and
(b) providing an assessment of the ALK fusion nucleic acid molecule or polypeptide.

Exemplary Embodiment 13: A method of detecting an ALK fusion nucleic acid molecule or polypeptide, the method comprising detecting in a sample from an individual having a cancer:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule.

Exemplary Embodiment 14: A method of detecting the presence or absence of a cancer in an individual, the method comprising:
(a) detecting the presence or absence of a cancer in a sample from the individual; and
(b) detecting in a sample from the individual the presence or absence of:
   (i) an ALK fusion nucleic acid molecule comprising a fusion between an ALK gene, or a portion thereof, and a gene listed in Table 1, or a portion thereof, or
   (ii) an ALK fusion polypeptide encoded by the ALK fusion nucleic acid molecule.

Exemplary Embodiment 15: The method of embodiment 14, comprising detecting the presence of the cancer in a sample from the individual.

Exemplary Embodiment 16: The method of embodiment 14 or embodiment 15, comprising detecting the presence of the ALK fusion nucleic acid molecule, or the ALK fusion polypeptide encoded by the ALK fusion nucleic acid molecule, in a sample from the individual.

Exemplary Embodiment 17: A method for monitoring progression or recurrence of a cancer in an individual, the method comprising:
(a) detecting, in a first sample obtained from the individual at a first time point, the presence or absence of: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule;
(b) detecting, in a second sample obtained from the individual at a second time point after the first time point, the presence or absence of: (i) an ALK fusion nucleic acid molecule comprising a fusion between an ALK gene, or a portion thereof, and a gene listed in Table 1, or a portion thereof, or (ii) an ALK fusion polypeptide encoded by the ALK fusion nucleic acid molecule; and
(c) providing an assessment of cancer progression or cancer recurrence in the individual based, at least in part, on the presence or absence of the ALK fusion nucleic acid molecule or the ALK fusion polypeptide in the first sample and/or in the second sample.

Exemplary Embodiment 18: The method of embodiment 17, wherein the presence of the ALK fusion nucleic acid molecule or the ALK fusion polypeptide in the first sample and/or in the second sample identifies the individual as having increased risk of cancer progression or cancer recurrence.

Exemplary Embodiment 19: The method of embodiment 17 or embodiment 18, further comprising selecting a treatment, administering a treatment, adjusting a treatment, adjusting the dose of a treatment, or applying a treatment to the individual based, at least in part, on detecting the presence of the ALK fusion nucleic acid molecule or the ALK fusion polypeptide in the first sample and/or in the second sample, wherein the treatment comprises an ALK-targeted therapy.

Exemplary Embodiment 20: A method of detecting an ALK fusion nucleic acid molecule, the method comprising:
(a) providing a plurality of nucleic acid molecules obtained from a sample from an individual having a cancer, wherein the plurality of nucleic acid molecules comprises nucleic acid molecules corresponding to an ALK fusion nucleic acid molecule comprising a fusion between an ALK gene, or a portion thereof, and a gene listed in Table 1, or a portion thereof;
(b) optionally, ligating one or more adapters onto one or more nucleic acid molecules from the plurality of nucleic acid molecules;
(c) optionally, amplifying the one or more ligated nucleic acid molecules from the plurality of nucleic acid molecules;
(d) optionally, capturing amplified nucleic acid molecules from the amplified nucleic acid molecules;
(e) sequencing, by a sequencer, the captured nucleic acid molecules to obtain a plurality of sequence reads that represent the captured nucleic acid molecules, wherein one or more of the plurality of sequence reads correspond to the ALK fusion nucleic acid molecule;
(f) analyzing the plurality of sequence reads; and
(g) based on the analysis, detecting the presence or absence of the ALK fusion nucleic acid molecule in the sample.

Exemplary Embodiment 21: The method of embodiment 20, further comprising receiving, at one or more processors, sequence read data for the plurality of sequence reads.

Exemplary Embodiment 22: The method of embodiment 21, wherein the analyzing the plurality of sequence reads comprises identifying, using the one or more processors, the presence or absence of sequence reads corresponding to the ALK fusion nucleic acid molecule.

Exemplary Embodiment 23: The method of any one of embodiments 20-22, wherein the amplified nucleic acid molecules are captured by hybridization with one or more bait molecules.

Exemplary Embodiment 24: A method of detecting an ALK fusion nucleic acid molecule, the method comprising:
(a) providing a sample from an individual having a cancer, wherein the sample comprises a plurality of nucleic acid molecules;
(b) preparing a nucleic acid sequencing library from the plurality of nucleic acid molecules in the sample;
(c) amplifying said library;
(d) selectively enriching for one or more nucleic acid molecules comprising nucleotide sequences corresponding to an ALK fusion nucleic acid molecule in said library to produce an enriched sample, wherein the ALK fusion nucleic acid molecule comprises a fusion between an ALK gene, or a portion thereof, and a gene listed in Table 1, or a portion thereof;
(e) sequencing the enriched sample, thereby producing a plurality of sequence reads;
(f) analyzing the plurality of sequence reads for the presence of the ALK fusion nucleic acid molecule; and
(g) detecting, based on the analyzing step, the presence or absence of the ALK fusion nucleic acid molecule in the sample from the individual.

Exemplary Embodiment 25: The method of any one of embodiments 20-24, wherein the plurality of nucleic acid molecules comprises a mixture of cancer nucleic acid molecules and non-cancer nucleic acid molecules.

Exemplary Embodiment 26: The method of embodiment 25, wherein the cancer nucleic acid molecules are derived from a tumor portion of a heterogeneous tissue biopsy sample, and the non-cancer nucleic acid molecules are derived from a normal portion of the heterogeneous tissue biopsy sample.

Exemplary Embodiment 27: The method of embodiment 25, wherein the sample comprises a liquid biopsy sample, and wherein the cancer nucleic acid molecules are derived from a circulating tumor DNA (ctDNA) fraction of the liquid biopsy sample, and the non-cancer nucleic acid molecules are derived from a non-tumor fraction of the liquid biopsy sample.

Exemplary Embodiment 28: The method of any one of embodiments 20-23 and 25-27, wherein the one or more adapters comprise amplification primers, flow cell adapter sequences, substrate adapter sequences, sample index sequences, or unique molecular identifier (UMI) sequences.

Exemplary Embodiment 29: The method of any one of embodiments 24-27, wherein the selectively enriching comprises: (a) combining one or more bait molecules with the library, thereby hybridizing the one or more bait molecules to one or more nucleic acid molecules comprising nucleotide sequences corresponding to the ALK fusion nucleic acid molecule and producing nucleic acid hybrids; and (b) isolating the nucleic acid hybrids to produce the enriched sample.

Exemplary Embodiment 30: The method of any one of embodiments 20-23 and 25-28, wherein the captured nucleic acid molecules are captured from the amplified nucleic acid molecules by hybridization to one or more bait molecules.

Exemplary Embodiment 31: The method of any one of embodiments 20-30, wherein the amplifying comprises performing a polymerase chain reaction (PCR) amplification technique, a non-PCR amplification technique, or an isothermal amplification technique.

Exemplary Embodiment 32: The method of any one of embodiments 20-31, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 33: The method of embodiment 32, wherein the sequencing comprises a massively parallel sequencing technique, and the massively parallel sequencing technique comprises next generation sequencing (NGS).

Exemplary Embodiment 34: The method of any one of embodiments 20-23, 25-28, and 30-33, wherein the sequencer comprises a next generation sequencer.

Exemplary Embodiment 35: The method of any one of embodiments 20-34, further comprising generating a molecular profile for the individual, based, at least in part, on detecting the presence or absence of the ALK fusion nucleic acid molecule.

Exemplary Embodiment 36: The method of embodiment 35, wherein the molecular profile for the individual further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Embodiment 37: The method of embodiment 35 or embodiment 36, wherein the molecular profile for the individual further comprises results from a nucleic acid sequencing-based test.

Exemplary Embodiment 38: The method of any one of embodiments 35-37, further comprising selecting a treatment, administering a treatment, or applying a treatment to the individual based on the generated molecular profile, wherein the treatment comprises an ALK-targeted therapy.

Exemplary Embodiment 39: The method of any one of embodiments 20-38, further comprising generating a report indicating the presence or absence of the ALK fusion nucleic acid molecule in the sample.

Exemplary Embodiment 40: The method of embodiment 21 or embodiment 22, further comprising generating, by the one or more processors, a report indicating the presence or absence of the ALK fusion nucleic acid molecule in the sample.

Exemplary Embodiment 41: The method of embodiment 39 or embodiment 40, further comprising transmitting the report to the individual, a caregiver, a healthcare provider, a physician, an oncologist, an electronic medical record system, a hospital, a clinic, a third-party payer, an insurance company, or a government office.

Exemplary Embodiment 42: The method of embodiment 41, wherein the report is transmitted via a computer network or a peer-to-peer connection.

Exemplary Embodiment 43: A method of identifying a candidate treatment for a cancer in an individual in need thereof, comprising performing DNA sequencing on a sample obtained from the individual to determine a sequencing mutation profile on a group of genes comprising one or more of ALK, one or more genes listed in Table 1, or any combination thereof, wherein the sequencing mutation profile identifies the presence or absence of an ALK fusion nucleic acid molecule, wherein the ALK fusion nucleic acid molecule comprises a fusion between an ALK gene, or a portion thereof, and a gene listed in Table 1, or a portion thereof.

Exemplary Embodiment 44: The method of embodiment 43, wherein the candidate treatment comprises an ALK-targeted therapy.

Exemplary Embodiment 45: The method of embodiment 43 or embodiment 44, wherein the presence of the ALK fusion nucleic acid molecule in the sample identifies the individual as one who may benefit from a treatment comprising an ALK-targeted therapy.

Exemplary Embodiment 46: The method of any one of embodiments 43-45, wherein the presence of the ALK fusion nucleic acid molecule in the sample predicts the individual to have longer survival when treated with a treatment comprising an ALK-targeted therapy, as compared to survival of an individual whose cancer does not comprise an ALK fusion nucleic acid molecule.

Exemplary Embodiment 47: The method of any one of embodiments 43-46, wherein the sequencing comprises use of a massively parallel sequencing (MPS) technique, whole genome sequencing (WGS), whole exome sequencing, targeted sequencing, direct sequencing, or a Sanger sequencing technique.

Exemplary Embodiment 48: The method of embodiment 47, wherein the sequencing comprises a massively parallel sequencing technique, and the massively parallel sequencing technique comprises next generation sequencing (NGS).

Exemplary Embodiment 49: The method of any one of embodiments 43-48, wherein the sequencing mutation profile identifies the presence or absence of a fragment of the ALK fusion nucleic acid molecule comprising a breakpoint or fusion junction.

Exemplary Embodiment 50: A method of treating or delaying progression of cancer, comprising:
(a) detecting in a sample from an individual having a cancer:
   (i) an ALK fusion nucleic acid molecule comprising a fusion between an ALK gene, or a portion thereof, and a gene listed in Table 1, or a portion thereof, or
   (ii) an ALK fusion polypeptide encoded by the ALK fusion nucleic acid molecule; and
(b) administering to the individual an effective amount of a treatment that comprises an ALK-targeted therapy.

Exemplary Embodiment 51: The method of any one of embodiments 1-50, wherein the ALK fusion nucleic acid molecule is an ALK fusion nucleic acid molecule listed in Table 2, and wherein the order of the genes in the fusion in the 5′ to 3′ direction is as listed in Table 2.

Exemplary Embodiment 52: The method of any one of embodiments 1-51, wherein the ALK fusion nucleic acid molecule is an ALK fusion nucleic acid molecule listed in Table 3, and wherein the ALK fusion nucleic acid molecule comprises or results from a corresponding 5′ breakpoint and/or 3′ breakpoint within the exons or introns as listed in Table 3.

Exemplary Embodiment 53: The method of any one of embodiments 1-52, wherein the ALK fusion nucleic acid molecule is an ALK fusion nucleic acid molecule listed in Table 4, and wherein the ALK fusion nucleic acid molecule comprises or results from a corresponding 5′ breakpoint within the chromosomal coordinates as listed in Table 4, and/or a corresponding 3′ breakpoint within the chromosomal coordinates as listed in Table 4.

Exemplary Embodiment 54: The method of any one of embodiments 1-53, wherein the ALK fusion nucleic acid molecule is an ALK fusion nucleic acid molecule listed in Table 5, and wherein the ALK fusion nucleic acid molecule comprises or results from a fusion of a corresponding 5′ exon as listed in Table 5, or a portion thereof, fused to a corresponding 3′ exon as listed in Table 5, or a portion thereof.

Exemplary Embodiment 55: The method of any one of embodiments 1-54, wherein the ALK fusion nucleic acid molecule is an ALK fusion nucleic acid molecule listed in Table 6, and wherein the ALK fusion nucleic acid molecule comprises, in the 5′ to 3′ direction, the corresponding exons or portions thereof as listed in Table 6.

Exemplary Embodiment 56: The method of any one of embodiments 1-55, wherein the ALK fusion nucleic acid molecule is an ALK fusion nucleic acid molecule listed in Table 7, and wherein the ALK fusion nucleic acid molecule comprises a corresponding nucleotide sequence as listed in Table 7, or a nucleotide sequence with at least about 70% homology thereto.

Exemplary Embodiment 57: The method of any one of embodiments 1-56, wherein the ALK fusion nucleic acid molecule encodes an ALK fusion polypeptide.

Exemplary Embodiment 58: The method of any one of embodiments 1-57, wherein the ALK fusion nucleic acid molecule comprises a nucleotide sequence encoding an ALK fusion polypeptide that comprises an amino acid sequence as listed in Table 8, or an amino acid sequence with at least about 70% homology thereto.

Exemplary Embodiment 59: The method of any one of embodiments 1-58, wherein the ALK fusion polypeptide encoded by the ALK fusion nucleic acid molecule comprises an ALK kinase domain, or a fragment of an ALK kinase domain having ALK kinase activity.

Exemplary Embodiment 60: The method of any one of embodiments 1-59, wherein the ALK fusion polypeptide encoded by the ALK fusion nucleic acid molecule has ALK kinase activity.

Exemplary Embodiment 61: The method of any one of embodiments 1-60, wherein the ALK fusion polypeptide encoded by the ALK fusion nucleic acid molecule has a constitutive ALK kinase activity.

Exemplary Embodiment 62: The method of any one of embodiments 1-61, wherein the ALK fusion polypeptide encoded by the ALK fusion nucleic acid molecule is oncogenic.

Exemplary Embodiment 63: The method of any one of embodiments 1-62, wherein the ALK fusion polypeptide encoded by the ALK fusion nucleic acid molecule promotes cancer cell survival, angiogenesis, cancer cell proliferation, and any combination thereof.

Exemplary Embodiment 64: The method of any one of embodiments 1-63, wherein the ALK fusion polypeptide encoded by the ALK fusion nucleic acid molecule is capable of dimerizing with an ALK polypeptide or with another ALK fusion polypeptide in a cancer cell.

Exemplary Embodiment 65: The method of any one of embodiments 1-64, wherein the ALK fusion polypeptide encoded by the ALK fusion nucleic acid molecule is an ALK fusion polypeptide listed in Table 8, and wherein the ALK fusion polypeptide comprises a corresponding amino acid sequence as listed in Table 8, or an amino acid sequence with at least about 70% homology thereto.

Exemplary Embodiment 66: The method of any one of embodiments 1-65, wherein the cancer is a carcinoma, a sarcoma, a lymphoma, a leukemia, a myeloma, a germ cell cancer, or a blastoma.

Exemplary Embodiment 67: The method of any one of embodiments 1-66, wherein the cancer is a solid tumor.

Exemplary Embodiment 68: The method of any one of embodiments 1-66, wherein the cancer is a hematologic malignancy.

Exemplary Embodiment 69: The method of any one of embodiments 1-66, wherein the cancer is a lymphoma.

Exemplary Embodiment 70: The method of any one of embodiments 1-66, wherein the cancer is a non-small cell lung carcinoma, a leiomyosarcoma, a thyroid carcinoma, a colorectal cancer, a pancreatic cancer, or a malignant peritoneal mesothelioma.

Exemplary Embodiment 71: The method of any one of embodiments 1-66, wherein the cancer is a B cell cancer, multiple myeloma, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Exemplary Embodiment 72: The method of any one of embodiments 1-66, wherein the cancer is an anus squamous cell carcinoma, brain glioblastoma (GBM), breast cancer, breast carcinoma, breast invasive ductal carcinoma (IDC), colon adenocarcinoma (CRC), esophagus adenocarcinoma, fallopian tube serous carcinoma, gallbladder adenocarcinoma, gallbladder carcinoma, gastroesophageal junction adenocarcinoma, lung adenocarcinoma, lung non-small cell lung carcinoma, lung non-small cell lung carcinoma, lymph node Castleman's disease, lymph node lymphoma T-cell, ovary clear cell carcinoma, ovary endometrioid adenocarcinoma, ovary epithelial carcinoma, ovary high grade serous carcinoma, ovary serous carcinoma, pancreas cancer, pancreas ductal adenocarcinoma, pediatric bone osteosarcoma, bone osteosarcoma, pediatric skin melanoma, skin melanoma, pediatric soft tissue sarcoma, soft tissue sarcoma, pediatric soft tissue sarcoma undifferentiated, soft tissue sarcoma undifferentiated, peritoneum serous carcinoma, prostate acinar adenocarcinoma, small intestine adenocarcinoma, soft tissue leiomyosarcoma, soft tissue liposarcoma, thyroid papillary carcinoma, unknown primary adenocarcinoma, unknown primary carcinoma, unknown primary malignant neoplasm, unknown primary myoepithelial carcinoma, uterus carcinosarcoma, uterus endometrial adenocarcinoma endometrioid, uterus leiomyosarcoma, or vulva squamous cell carcinoma (SCC).

Exemplary Embodiment 73: The method of any one of embodiments 51-65, wherein the ALK fusion nucleic acid molecule is an ALK fusion nucleic acid molecule listed in Table 9, or the ALK fusion polypeptide is an ALK fusion polypeptide listed in Table 9, and wherein the cancer is the corresponding cancer as listed in Table 9.

Exemplary Embodiment 74: The method of any one of embodiments 1-73, wherein the cancer is metastatic.

Exemplary Embodiment 75: The method of embodiment 74, wherein the cancer has metastasized to the brain of the individual.

Exemplary Embodiment 76: The method of embodiment 74, wherein the individual has an intracranial metastasis of the cancer.

Exemplary Embodiment 77: The method of embodiment 74, wherein the individual has an extracranial metastasis of the cancer.

Exemplary Embodiment 78: The method of any one of embodiments 1-74, wherein the cancer has not metastasized to the brain of the individual.

Exemplary Embodiment 79: The method of any one of embodiments 1-74, wherein the individual does not have an intracranial metastasis of the cancer.

Exemplary Embodiment 80: The method of any one of embodiments 1-73, wherein the individual does not have an extracranial metastasis of the cancer.

Exemplary Embodiment 81: The method of any one of embodiments 1-11, 19, 38, and 44-80, wherein the ALK-targeted therapy comprises one or more of a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof.

Exemplary Embodiment 82: The method of any one of embodiments 1-11, 19, 38, and 44-81, wherein the ALK-targeted therapy is a kinase inhibitor.

Exemplary Embodiment 83: The method of embodiment 82, wherein the ALK-targeted therapy is a tyrosine kinase inhibitor.

Exemplary Embodiment 84: The method of embodiment 82 or embodiment 83, wherein the ALK-targeted therapy is kinase inhibitor that inhibits the kinase activity of an ALK polypeptide.

Exemplary Embodiment 85: The method of any one of embodiments 82-84, wherein the ALK-targeted therapy is a multi-kinase inhibitor or an ALK-specific inhibitor.

Exemplary Embodiment 86: The method of any one of embodiments 82-85, wherein the ALK-targeted therapy comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A.

Exemplary Embodiment 87: The method of embodiment 81, wherein the nucleic acid inhibits the expression of the ALK fusion nucleic acid molecule or polypeptide.

Exemplary Embodiment 88: The method of embodiment 87, wherein the nucleic acid is a double-stranded RNA (dsRNA), a small interfering RNA (siRNA), or a small hairpin RNA (shRNA).

Exemplary Embodiment 89: The method of embodiment 81, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 90: The method of any one of embodiments 1-89, wherein the individual has received a prior anti-cancer treatment, or is being treated with an anti-cancer treatment.

Exemplary Embodiment 91: The method of embodiment 90, wherein the ALK fusion nucleic acid molecule, and/or the ALK fusion polypeptide encoded by the ALK fusion nucleic acid molecule, confers resistance of the cancer to the anti-cancer treatment.

Exemplary Embodiment 92: The method of embodiment 90 or embodiment 91, wherein the anti-cancer treatment is a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for cancer being tested in a clinical trial, an immunotherapy, a chemotherapy, a targeted therapy, a non-ALK-targeted anti-cancer therapy, or any combination thereof.

Exemplary Embodiment 93: The method of embodiment 92, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 94: The method of embodiment 92, wherein the nucleic acid comprises a double-stranded RNA (dsRNA), a small interfering RNA (siRNA), or a small hairpin RNA (shRNA).

Exemplary Embodiment 95: The method of any one of embodiments 1-89, wherein the cancer has not been previously treated.

Exemplary Embodiment 96: The method of any one of embodiments 1-11, 19, 38, 44-89, and 95, wherein the ALK-targeted therapy is a first-line or front-line treatment.

Exemplary Embodiment 97: The method of embodiment 95 or embodiment 96, wherein the ALK-targeted therapy is brigatinib.

Exemplary Embodiment 98: The method of embodiment 95 or embodiment 96, wherein the ALK-targeted therapy is crizotinib.

Exemplary Embodiment 99: The method of embodiment 95 or embodiment 96, wherein the ALK-targeted therapy is lorlatinib.

Exemplary Embodiment 100: The method of embodiment 95 or embodiment 96, wherein the ALK-targeted therapy is a kinase inhibitor in combination with a PD-1- or a PD-L1-targeted agent.

Exemplary Embodiment 101: The method of embodiment 100, wherein the kinase inhibitor is a tyrosine kinase inhibitor.

Exemplary Embodiment 102: The method of embodiment 100 or embodiment 101, wherein the kinase inhibitor inhibits the kinase activity of an ALK polypeptide.

Exemplary Embodiment 103: The method of any one of embodiments 100-102, wherein the kinase inhibitor is a multi-kinase inhibitor or an ALK-specific inhibitor.

Exemplary Embodiment 104: The method of any one of embodiments 100-103, wherein the kinase inhibitor comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, and TAE684 (NVP-TAE684).

Exemplary Embodiment 105: The method of any one of embodiments 100-104, wherein the PD-1-targeted agent is a PD-1 inhibitor.

Exemplary Embodiment 106: The method of embodiment 105, wherein the PD-1 inhibitor comprises one or more of nivolumab, pembrolizumab, cemiplimab, or dostarlimab.

Exemplary Embodiment 107: The method of any one of embodiments 100-104, wherein the PD-L1-targeted agent is a PD-L1 inhibitor.

Exemplary Embodiment 108: The method of embodiment 107, wherein the PD-L1 inhibitor comprises one or more of atezolizumab, avelumab, or durvalumab.

Exemplary Embodiment 109: The method of embodiment 100, wherein the ALK-targeted therapy is alectinib in combination with a PD-1- or a PD-L1-targeted agent.

Exemplary Embodiment 110: The method of embodiment 109, wherein the ALK-targeted therapy is alectinib in combination with atezolizumab.

Exemplary Embodiment 111: The method of any one of embodiments 95-110, wherein the cancer is a non-small cell lung carcinoma.

Exemplary Embodiment 112: The method of any one of embodiments 1-89, wherein the cancer has not been previously treated with crizotinib.

Exemplary Embodiment 113: The method of any one of embodiments 1-89, wherein the cancer is crizotinib-naïve.

Exemplary Embodiment 114: The method of embodiment 112 or embodiment 113, wherein the ALK-targeted therapy is brigatinib.

Exemplary Embodiment 115: The method of any one of embodiments 1-89, wherein the cancer is kinase inhibitor-naïve.

Exemplary Embodiment 116: The method of any one of embodiments 1-89, wherein the cancer has not been previously treated with a kinase inhibitor.

Exemplary Embodiment 117: The method of embodiment 115 or embodiment 116, wherein the ALK-targeted therapy is crizotinib or alectinib.

Exemplary Embodiment 118: The method of embodiment 115 or embodiment 116, wherein the ALK-targeted therapy is alectinib.

Exemplary Embodiment 119: The method of embodiment 115 or embodiment 116, wherein the ALK-targeted therapy is ceritinib.

Exemplary Embodiment 120: The method of any one of embodiments 115-119, wherein the cancer is a non-small cell lung carcinoma

Exemplary Embodiment 121: The method of any one of embodiments 115-120, wherein the cancer is a kinase inhibitor-naïve non-small cell lung carcinoma.

Exemplary Embodiment 122: The method of any one of embodiments 1-89, wherein the cancer has been previously treated with a kinase inhibitor.

Exemplary Embodiment 123: The method of any one of embodiments 1-89 and 122, wherein the cancer progressed on a prior treatment with a kinase inhibitor.

Exemplary Embodiment 124: The method of any one of embodiments 1-89 and 122-123, wherein the cancer is refractory to a prior kinase inhibitor treatment.

Exemplary Embodiment 125: The method of any one of embodiments 1-89 and 122-124, wherein the cancer progressed on a prior treatment with a chemotherapy and a kinase inhibitor.

Exemplary Embodiment 126: The method of any one of embodiments 122-125, wherein the kinase inhibitor is a tyrosine kinase inhibitor.

Exemplary Embodiment 127: The method of any one of embodiments 122-126, wherein the kinase inhibitor inhibits the kinase activity of an ALK polypeptide.

Exemplary Embodiment 128: The method of any one of embodiments 122-127, wherein the kinase inhibitor is a multi-kinase inhibitor or an ALK-specific inhibitor.

Exemplary Embodiment 129: The method of any one of embodiments 122-124, wherein the ALK-targeted therapy is lorlatinib.

Exemplary Embodiment 130: The method of any one of embodiments 122-124, wherein the kinase inhibitor is crizotinib, and the ALK-targeted therapy is alectinib, ceritinib or brigatinib.

Exemplary Embodiment 131: The method of any one of embodiments 122-124, wherein the kinase inhibitor is alectinib, and the ALK-targeted therapy is brigatinib.

Exemplary Embodiment 132: The method of any one of embodiments 122-124, wherein the cancer progressed on a prior crizotinib treatment, and wherein the ALK-targeted therapy comprises continued treatment with crizotinib.

Exemplary Embodiment 133: The method of any one of embodiments 122-124, wherein the cancer progressed on a prior lorlatinib treatment, and wherein the ALK-targeted therapy comprises continued treatment with lorlatinib.

Exemplary Embodiment 134: The method of any one of embodiments 125-128, wherein the kinase inhibitor is crizotinib, and the ALK-targeted therapy is ceritinib.

Exemplary Embodiment 135: The method of any one of embodiments 122-134, wherein the cancer is a non-small cell lung carcinoma.

Exemplary Embodiment 136: The method of any one of embodiments 1-67 and 70-89, wherein the cancer is a solid tumor, and wherein the ALK-targeted therapy is entrectinib.

Exemplary Embodiment 137: The method of embodiment 136, wherein the cancer is a non-small cell lung carcinoma, a renal cell carcinoma, a colorectal cancer, or an inflammatory myofibroblastic tumor.

Exemplary Embodiment 138: The method of any one of embodiments 1-11, 19, 38, and 44-89, wherein the ALK-targeted therapy is a fourth line treatment.

Exemplary Embodiment 139: The method of embodiment 138, wherein the ALK-targeted therapy is lorlatinib.

Exemplary Embodiment 140: The method of embodiment 138 or embodiment 139, wherein the cancer is an inflammatory myofibroblastic sarcoma, optionally, wherein the inflammatory myofibroblastic sarcoma is metastatic.

Exemplary Embodiment 141: The method of any one of embodiments 138-140, wherein the individual has leptomeningeal disease.

Exemplary Embodiment 142: The method of any one of embodiments 1-141, wherein the cancer further comprises one or more mutations in an ALK kinase domain encoded by an ALK gene.

Exemplary Embodiment 143: The method of any one of embodiments 1-142, wherein the ALK fusion nucleic acid molecule encodes an ALK fusion polypeptide comprising an ALK kinase domain, or a portion thereof, comprising one or more mutations in the ALK kinase domain.

Exemplary Embodiment 144: The method of any one of embodiments 1-19 and 50-142, wherein the ALK fusion polypeptide encoded by the ALK fusion nucleic acid molecule comprises an ALK kinase domain, or a portion thereof, comprising one or more mutations in the ALK kinase domain.

Exemplary Embodiment 145: The method of any one of embodiments 142-144, wherein the one or more mutations in the ALK kinase domain comprise an amino acid substitution at amino acid position G1202 of the ALK kinase domain, optionally wherein the amino acid substitution is a G1202R amino acid substitution.

Exemplary Embodiment 146: The method of any one of embodiments 142-145, wherein the ALK-targeted therapy is lorlatinib.

Exemplary Embodiment 147: The method of any one of embodiments 1-89, wherein the cancer was previously treated with crizotinib, and wherein the ALK-targeted therapy is brigatinib.

Exemplary Embodiment 148: The method of embodiment 147, wherein the cancer is a non-small cell lung carcinoma.

Exemplary Embodiment 149: The method of any one of embodiments 1-89, wherein the cancer is a colorectal cancer, and wherein the ALK-targeted therapy is ceritinib.

Exemplary Embodiment 150: The method of any one of embodiments 1-89, wherein the cancer is a pancreatic cancer, and wherein the ALK-targeted therapy is ceritinib.

Exemplary Embodiment 151: The method of embodiment 150, wherein the cancer is a pancreatic ductal adenocarcinoma.

Exemplary Embodiment 152: The method of any one of embodiments 1-11, 19, 38, and 44-89, wherein the ALK-targeted therapy is a kinase inhibitor in combination with a PD-1- or a PD-L1-targeted agent.

Exemplary Embodiment 153: The method of embodiment 152, wherein the kinase inhibitor is a tyrosine kinase inhibitor.

Exemplary Embodiment 154: The method of embodiment 152 or embodiment 153, wherein the kinase inhibitor inhibits the kinase activity of an ALK polypeptide.

Exemplary Embodiment 155: The method of any one of embodiments 152-154, wherein the kinase inhibitor is a multi-kinase inhibitor or an ALK-specific inhibitor.

Exemplary Embodiment 156: The method of any one of embodiments 152-155, wherein the kinase inhibitor comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, and TAE684 (NVP-TAE684).

Exemplary Embodiment 157: The method of any one of embodiments 152-156, wherein the PD-1-targeted agent is a PD-1 inhibitor.

Exemplary Embodiment 158: The method of embodiment 157, wherein the PD-1 inhibitor comprises one or more of nivolumab, pembrolizumab, cemiplimab, or dostarlimab.

Exemplary Embodiment 159: The method of any one of embodiments 152-156, wherein the PD-L1-targeted agent is a PD-L1 inhibitor.

Exemplary Embodiment 160: The method of embodiment 159, wherein the PD-L1 inhibitor comprises one or more of atezolizumab, avelumab, or durvalumab.

Exemplary Embodiment 161: The method of embodiment 152, wherein the ALK-targeted therapy is lorlatinib in combination with avelumab.

Exemplary Embodiment 162: The method of any one of embodiments 152-161, wherein the cancer is a non-small cell lung carcinoma.

Exemplary Embodiment 163: The method of any one of embodiments 1-11, 19, 38, and 44-162, wherein the treatment or the one or more treatment options further comprise an additional anti-cancer therapy.

Exemplary Embodiment 164: The method of embodiment 163, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof.

Exemplary Embodiment 165: The method of embodiment 164, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, a macrophage-based therapy, an induced pluripotent stem cell-based therapy, a B cell-based therapy, or a dendritic cell (DC)-based therapy.

Exemplary Embodiment 166: The method of embodiment 163, wherein the additional anti-cancer therapy comprises one or more of a heat shock protein 90 inhibitor, an EGFR inhibitor, a SHP2 inhibitor, a MEK inhibitor, an IGF-1R inhibitor, a vascular endothelial growth factor (VEGF)-targeted therapy, or an mTOR inhibitor.

Exemplary Embodiment 167: The method of embodiment 164, wherein the nucleic acid comprises a double-stranded RNA (dsRNA), a small interfering RNA (siRNA), or a small hairpin RNA (shRNA).

Exemplary Embodiment 168: The method of any one of embodiments 1-167, further comprising obtaining the sample from the individual.

Exemplary Embodiment 169: The method of any one of embodiments 1-168, wherein the sample is obtained from the cancer.

Exemplary Embodiment 170: The method of any one of embodiments 1-169, wherein the sample comprises a tissue biopsy sample, a liquid biopsy sample, or a normal control.

Exemplary Embodiment 171: The method of embodiment 170, wherein the sample is from a tumor biopsy, tumor specimen, or circulating tumor cell.

Exemplary Embodiment 172: The method of any one of embodiments 1-170, wherein the sample is a liquid biopsy sample and comprises blood, plasma, cerebrospinal fluid, sputum, stool, urine, or saliva.

Exemplary Embodiment 173: The method of any one of embodiments 1-172, wherein the sample comprises cells and/or nucleic acids from the cancer.

Exemplary Embodiment 174: The method of embodiment 173, wherein the sample comprises mRNA, DNA, circulating tumor DNA (ctDNA), cell-free DNA, or cell-free RNA from the cancer.

Exemplary Embodiment 175: The method of embodiment 172, wherein the sample is a liquid biopsy sample and comprises circulating tumor cells (CTCs).

Exemplary Embodiment 176: The method of embodiment 172, wherein the sample is a liquid biopsy sample and comprises cell-free DNA (cfDNA), circulating tumor DNA (ctDNA), or any combination thereof.

Exemplary Embodiment 177: The method of any one of embodiments 1-176, comprising acquiring knowledge of or detecting the ALK fusion nucleic acid molecule or the ALK fusion polypeptide encoded by the ALK fusion nucleic acid molecule in a tissue biopsy sample, in a liquid biopsy sample, or in both a tissue biopsy sample and a liquid biopsy sample, from the individual.

Exemplary Embodiment 178: The method of any one of embodiments 4-11 and 51-177, wherein the acquiring knowledge of the ALK fusion nucleic acid molecule or the ALK fusion polypeptide encoded by the ALK fusion nucleic acid molecule comprises detecting the ALK fusion nucleic acid molecule or polypeptide in the sample.

Exemplary Embodiment 179: The method of any one of embodiments 1-3, 12-42, and 50-178, wherein detecting the ALK fusion nucleic acid molecule in the sample comprises detecting a fragment of the ALK fusion nucleic acid molecule comprising a breakpoint or fusion junction between the ALK gene, or the portion thereof, and the gene listed in Table 1, or the portion thereof.

Exemplary Embodiment 180: The method of any one of embodiments 1-3, 12-19, and 50-179, wherein the ALK fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing.

Exemplary Embodiment 181: The method of embodiment 180, wherein the sequencing comprises a massively parallel sequencing (MPS) technique, whole genome sequencing (WGS), whole exome sequencing, targeted sequencing, direct sequencing, or a Sanger sequencing technique; and optionally wherein the massively parallel sequencing (MPS) technique comprises next-generation sequencing (NGS).

Exemplary Embodiment 182: The method of any one of embodiments 1-3, 12-19, and 50-178, wherein detecting the ALK fusion polypeptide comprises detecting a portion of the ALK fusion polypeptide that is encoded by a fragment of the ALK fusion nucleic acid molecule that comprises a breakpoint or fusion junction between the ALK gene, or the portion thereof, and the gene listed in Table 1, or the portion thereof.

Exemplary Embodiment 183: The method of any one of embodiments 1-3, 12-19, 50-178, and 182, wherein the ALK fusion polypeptide is detected in the sample by one or more of: immunoblotting, enzyme linked immunosorbent assay (ELISA), immunohistochemistry, or mass spectrometry.

Exemplary Embodiment 184: The method of any one of embodiments 1-3, 12-19, and 50-181, further comprising selectively enriching for one or more nucleic acids in the sample comprising nucleotide sequences corresponding to the ALK fusion nucleic acid molecule; wherein the selectively enriching produces an enriched sample.

Exemplary Embodiment 185: The method of embodiment 184, wherein the selectively enriching comprises: (a) combining one or more bait molecules with the sample, thereby hybridizing the one or more bait molecules to one or more nucleic acids in the sample comprising nucleotide sequences corresponding to the ALK fusion nucleic acid molecule and producing nucleic acid hybrids; and (b) isolating the nucleic acid hybrids to produce the enriched sample.

Exemplary Embodiment 186: The method of any one of embodiments 23, 29, 30, and 185, wherein the one or more bait molecules comprise a capture nucleic acid molecule configured to hybridize to a nucleotide sequence corresponding to the ALK fusion nucleic acid molecule.

Exemplary Embodiment 187: The method of embodiment 186, wherein the capture nucleic acid molecule comprises between about 10 and about 30 nucleotides, between about 50 and about 1000 nucleotides, between about 100 and about 500 nucleotides, between about 100 and about 300 nucleotides, or between about 100 and about 200 nucleotides.

Exemplary Embodiment 188: The method of any one of embodiments 23, 29, 30, and 185-187, wherein the one or more bait molecules are conjugated to an affinity reagent or to a detection reagent.

Exemplary Embodiment 189: The method of embodiment 188, wherein the affinity reagent is an antibody, an antibody fragment, or biotin, or wherein the detection reagent is a fluorescent marker.

Exemplary Embodiment 190: The method of any one of embodiments 186-189, wherein the capture nucleic acid molecule comprises a DNA, RNA, or mixed DNA/RNA molecule.

Exemplary Embodiment 191: The method of embodiment 24 or embodiment 184, wherein the selectively enriching comprises amplifying the one or more nucleic acid molecules comprising nucleotide sequences corresponding to the ALK fusion nucleic acid molecule using a polymerase chain reaction (PCR) to produce an enriched sample.

Exemplary Embodiment 192: The method of any one of embodiments 184-191, further comprising sequencing the enriched sample.

Exemplary Embodiment 193: The method of any one of embodiments 1-192, further comprising acquiring knowledge of or detecting in a sample from the individual a base substitution, a short insertion/deletion (indel), a copy number alteration, or a genomic rearrangement in one or more genes.

Exemplary Embodiment 194: The method of any one of embodiments 1-193, wherein the individual is a human.

Exemplary Embodiment 195: A kit comprising a probe or bait for detecting an ALK fusion nucleic acid molecule comprising a fusion between an ALK gene, or a portion thereof, and a gene listed in Table 1, or a portion thereof.

Exemplary Embodiment 196: A nucleic acid encoding an ALK fusion nucleic acid molecule, or a fragment thereof, comprising a fusion between an ALK gene, or a portion thereof, and a gene listed in Table 1, or a portion thereof.

Exemplary Embodiment 197: A vector comprising the nucleic acid of embodiment 196.

Exemplary Embodiment 198: A host cell comprising the vector of embodiment 197.

Exemplary Embodiment 199: An antibody or antibody fragment that specifically binds to an ALK fusion polypeptide, or to a portion thereof, wherein the ALK fusion polypeptide is encoded by an ALK fusion nucleic acid molecule comprising a fusion between an ALK gene, or a portion thereof, and a gene listed in Table 1, or a portion thereof.

Exemplary Embodiment 200: A kit comprising the antibody or antibody fragment of embodiment 199.

Exemplary Embodiment 201: In vitro use of one or more oligonucleotides for detecting an ALK fusion nucleic acid molecule comprising a fusion between an ALK gene, or a portion thereof, and a gene listed in Table 1, or a portion thereof.

Exemplary Embodiment 202: A kit comprising one or more oligonucleotides for detecting an ALK fusion nucleic acid molecule comprising a fusion between an ALK gene, or a portion thereof, and a gene listed in Table 1, or a portion thereof.

Exemplary Embodiment 203: A system, comprising:
a memory configured to store one or more program instructions; and
one or more processors configured to execute the one or more program instructions, the one or more program instructions when executed by the one or more processors are configured to:
   (a) obtain a plurality of sequence reads of one or more nucleic acid molecules, wherein the one or more nucleic acid molecules are derived from a sample obtained from an individual;
   (b) analyze the plurality of sequence reads for the presence of an ALK fusion nucleic acid molecule comprising a fusion between an ALK gene, or a portion thereof, and a gene listed in Table 1, or a portion thereof; and
   (c) detect, based on the analyzing, the ALK fusion nucleic acid molecule in the sample.

Exemplary Embodiment 204: A non-transitory computer readable storage medium comprising one or more programs executable by one or more computer processors for performing a method, comprising:
(a) obtaining, using the one or more processors, a plurality of sequence reads of one or more nucleic acid molecules, wherein the one or more nucleic acid molecules are derived from a sample obtained from an individual;
(b) analyzing, using the one or more processors, the plurality of sequence reads for the presence of an ALK fusion nucleic acid molecule comprising a fusion between an ALK gene, or a portion thereof, and a gene listed in Table 1, or a portion thereof; and
(c) detecting, using the one or more processors and based on the analyzing, the ALK fusion nucleic acid molecule in the sample.

Exemplary Embodiment 205: The system of embodiment 203, or the non-transitory computer readable storage medium of embodiment 204, wherein the sample is from an individual having a cancer.

Exemplary Embodiment 206: The system of embodiment 203 or embodiment 205, or the non-transitory computer readable storage medium of embodiment 204 or embodiment 205, wherein the plurality of sequence reads is obtained by sequencing, whole exome sequencing, whole genome sequencing, gene-targeted sequencing, or next-generation sequencing.

Exemplary Embodiment 207: The system of any one of embodiments 203 and 205-206, or the non-transitory computer readable storage medium of any one of embodiments 204-206, wherein the ALK fusion nucleic acid molecule is an ALK fusion nucleic acid molecule listed in Table 2, and wherein the order of the genes in the fusion in the 5′ to 3′ direction is as listed in Table 2.

Exemplary Embodiment 208: The system of any one of embodiments 203 and 205-207, or the non-transitory computer readable storage medium of any one of embodiments 204-207, wherein the ALK fusion nucleic acid molecule is an ALK fusion nucleic acid molecule listed in Table 3, and wherein the ALK fusion nucleic acid molecule comprises or results from a corresponding 5′ breakpoint and/or 3′ breakpoint within the exons or introns as listed in Table 3.

Exemplary Embodiment 209: The system of any one of embodiments 203 and 205-208, or the non-transitory computer readable storage medium of any one of embodiments 204-208, wherein the ALK fusion nucleic acid molecule is an ALK fusion nucleic acid molecule listed in Table 4, and wherein the ALK fusion nucleic acid molecule comprises or results from a corresponding 5′ breakpoint within the chromosomal coordinates as listed in Table 4, and/or a corresponding 3′ breakpoint within the chromosomal coordinates as listed in Table 4.

Exemplary Embodiment 210: The system of any one of embodiments 203 and 205-209, or the non-transitory computer readable storage medium of any one of embodiments 204-209, wherein the ALK fusion nucleic acid molecule is an ALK fusion nucleic acid molecule listed in Table 5, and wherein the ALK fusion nucleic acid molecule comprises or results from a fusion of a corresponding 5′ exon as listed in Table 5, or a portion thereof, fused to a corresponding 3′ exon as listed in Table 5, or a portion thereof.

Exemplary Embodiment 211: The system of any one of embodiments 203 and 205-210, or the non-transitory computer readable storage medium of any one of embodiments 204-210, wherein the ALK fusion nucleic acid molecule is an ALK fusion nucleic acid molecule listed in Table 6, and wherein the ALK fusion nucleic acid molecule comprises, in the 5′ to 3′ direction, the corresponding exons or portions thereof as listed in Table 6.

Exemplary Embodiment 212: The system of any one of embodiments 203 and 205-211, or the non-transitory computer readable storage medium of any one of embodiments 204-211, wherein the ALK fusion nucleic acid molecule is an ALK fusion nucleic acid molecule listed in Table 7, and wherein the ALK fusion nucleic acid molecule comprises a corresponding nucleotide sequence as listed in Table 7, or a nucleotide sequence with at least about 70% homology thereto.

Exemplary Embodiment 213: The system of any one of embodiments 203 and 205-212, or the non-transitory computer readable storage medium of any one of embodiments 204-212, wherein the ALK fusion nucleic acid molecule encodes an ALK fusion polypeptide.

Exemplary Embodiment 214: The system of any one of embodiments 203 and 205-213, or the non-transitory computer readable storage medium of any one of embodiments 204-213, wherein the ALK fusion nucleic acid molecule comprises a nucleotide sequence encoding an ALK fusion polypeptide that comprises an amino acid sequence as listed in Table 8, or an amino acid sequence with at least about 70% homology thereto.

Exemplary Embodiment 215: The system or the non-transitory computer readable storage medium of any one of embodiments 213-214, wherein the encoded ALK fusion polypeptide comprises an ALK kinase domain, or a fragment of an ALK kinase domain, having ALK kinase activity.

Exemplary Embodiment 216: The system or the non-transitory computer readable storage medium of any one of embodiments 213-215, wherein the encoded ALK fusion polypeptide has ALK kinase activity.

Exemplary Embodiment 217: The system or the non-transitory computer readable storage medium of any one of embodiments 213-216, wherein the encoded ALK fusion polypeptide has a constitutive ALK kinase activity.

Exemplary Embodiment 218: The system or the non-transitory computer readable storage medium of any one of embodiments 213-217, wherein the encoded ALK fusion polypeptide is oncogenic.

Exemplary Embodiment 219: The system or the non-transitory computer readable storage medium of any one of embodiments 213-218, wherein the encoded ALK fusion polypeptide promotes cancer cell survival, angiogenesis, cancer cell proliferation, and any combination thereof.

Exemplary Embodiment 220: The system or the non-transitory computer readable storage medium of any one of embodiments 213-219, wherein the encoded ALK fusion polypeptide is capable of dimerizing with an ALK polypeptide or with another ALK fusion polypeptide in a cancer cell.

Exemplary Embodiment 221: The system or the non-transitory computer readable storage medium of any one of embodiments 213-220, wherein the encoded ALK fusion polypeptide is an ALK fusion polypeptide listed in Table 8, and wherein the encoded ALK fusion polypeptide comprises a corresponding amino acid sequence as listed in Table 8, or an amino acid sequence with at least about 70% homology thereto.

Exemplary Embodiment 222: The system or the non-transitory computer readable storage medium of any one of embodiments 205-221, wherein the cancer is a carcinoma, a sarcoma, a lymphoma, a leukemia, a myeloma, a germ cell cancer, or a blastoma.

Exemplary Embodiment 223: The system or the non-transitory computer readable storage medium of any one of embodiments 205-222, wherein the cancer is a solid tumor.

Exemplary Embodiment 224: The system or the non-transitory computer readable storage medium of any one of embodiments 205-222, wherein the cancer is a hematologic malignancy.

Exemplary Embodiment 225: The system or the non-transitory computer readable storage medium of any one of embodiments 205-222, wherein the cancer is a lymphoma.

Exemplary Embodiment 226: The system or the non-transitory computer readable storage medium of any one of embodiments 205-222, wherein the cancer is a non-small cell lung carcinoma, a leiomyosarcoma, a thyroid carcinoma, a colorectal cancer, a pancreatic cancer, or a malignant peritoneal mesothelioma.

Exemplary Embodiment 227: The system or the non-transitory computer readable storage medium of any one of embodiments 205-222, wherein the cancer is a B cell cancer, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, multiple myeloma (MM), myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor.

Exemplary Embodiment 228: The system or the non-transitory computer readable storage medium of any one of embodiments 205-222, wherein the cancer is an anus squamous cell carcinoma, brain glioblastoma (GBM), breast cancer, breast carcinoma, breast invasive ductal carcinoma (IDC), colon adenocarcinoma (CRC), esophagus adenocarcinoma, fallopian tube serous carcinoma, gallbladder adenocarcinoma, gallbladder carcinoma, gastroesophageal junction adenocarcinoma, lung adenocarcinoma, lung non-small cell lung carcinoma, lung non-small cell lung carcinoma, lymph node Castleman's disease, lymph node lymphoma T-cell, ovary clear cell carcinoma, ovary endometrioid adenocarcinoma, ovary epithelial carcinoma, ovary high grade serous carcinoma, ovary serous carcinoma, pancreas cancer, pancreas ductal adenocarcinoma, pediatric bone osteosarcoma, bone osteosarcoma, pediatric skin melanoma, skin melanoma, pediatric soft tissue sarcoma, soft tissue sarcoma, pediatric soft tissue sarcoma undifferentiated, soft tissue sarcoma undifferentiated, peritoneum serous carcinoma, prostate acinar adenocarcinoma, small intestine adenocarcinoma, soft tissue leiomyosarcoma, soft tissue liposarcoma, thyroid papillary carcinoma, unknown primary adenocarcinoma, unknown primary carcinoma, unknown primary malignant neoplasm, unknown primary myoepithelial carcinoma, uterus carcinosarcoma, uterus endometrial adenocarcinoma endometrioid, uterus leiomyosarcoma, or vulva squamous cell carcinoma (SCC).

Exemplary Embodiment 229: The system or the non-transitory computer readable storage medium of any one of embodiments 207-222, wherein the ALK fusion nucleic acid molecule is an ALK fusion nucleic acid molecule listed in Table 9, or the encoded ALK fusion polypeptide is an ALK fusion polypeptide listed in Table 9, and wherein the cancer is the corresponding cancer as listed in Table 9.

Exemplary Embodiment 230: The system of any one of embodiments 203 and 205-229, or the non-transitory computer readable storage medium of any one of embodiments 204-229, wherein the plurality of sequence reads is obtained by sequencing; optionally wherein the sequencing comprises use of a massively parallel sequencing (MPS) technique, whole genome sequencing (WGS), whole exome sequencing, targeted sequencing, direct sequencing, or a Sanger sequencing technique; and optionally wherein the massively parallel sequencing technique comprises next generation sequencing (NGS).

Exemplary Embodiment 231: The system of any one of embodiments 203 and 205-230, wherein the one or more program instructions when executed by the one or more processors are further configured to generate, based at least in part on the detecting, a molecular profile for the sample.

Exemplary Embodiment 232: The non-transitory computer readable storage medium of any one of embodiments 204-230, wherein the method further comprises generating, based at least in part on the detecting, a molecular profile for the sample.

Exemplary Embodiment 233: The system of embodiment 231, or the non-transitory computer readable storage medium of embodiment 232, wherein the individual is administered a treatment based at least in part on the molecular profile.

Exemplary Embodiment 234: The system of embodiment 231 or embodiment 233, or the non-transitory computer readable storage medium of embodiment 232 or embodiment 233, wherein the treatment comprises an ALK-targeted therapy.

Exemplary Embodiment 235: The system of any one of embodiments 231 and 233-234, or the non-transitory computer readable storage medium of any one of embodiments 232-234, wherein the molecular profile further comprises results from a comprehensive genomic profiling (CGP) test, a gene expression profiling test, a cancer hotspot panel test, a DNA methylation test, a DNA fragmentation test, an RNA fragmentation test, or any combination thereof.

Exemplary Embodiment 236: The system of any one of embodiments 231 and 233-235, or the non-transitory computer readable storage medium of any one of embodiments 232-235, wherein the molecular profile further comprises results from a nucleic acid sequencing-based test.

Exemplary Embodiment 237: An ALK-targeted therapy for use in a method of treating or delaying progression of cancer, wherein the method comprises administering the ALK-targeted therapy to an individual, wherein:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule, is detected in a sample obtained from the individual.

Exemplary Embodiment 238: An ALK-targeted therapy for use in the manufacture of a medicament for treating or delaying progression of cancer, wherein the medicament is to be administered to an individual, wherein:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule, is detected in a sample obtained from the individual.

The method steps of the invention(s) described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes. To the extent that any reference incorporated by reference conflicts with the instant disclosure, the instant disclosure shall control.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Pan-Cancer Landscape of ALK Gene Fusions

The anaplastic lymphoma kinase (ALK) gene encodes a receptor tyrosine kinase. ALK is a member of the insulin receptor superfamily. Its activation induces downstream pathways associated with cell survival, angiogenesis, and cell proliferation (Grande et al., Molecular cancer therapeutics, vol. 10, 4 (2011): 569-79). Gene fusions involving the ALK gene have been identified as being oncogenic (see, e.g., Hallberg et al., Annals of oncology, vol. 27 Suppl 3 (2016): iii4-iii15), and may confer sensitivity of cancer to certain anti-cancer therapies such as ALK inhibitors and other ALK-targeted therapies.

This Example describes a comprehensive characterization of the pan-cancer landscape of gene fusions involving the anaplastic lymphoma kinase (ALK).

ALK gene fusions were identified in samples from patients with various cancer types using comprehensive genomic profiling (CGP), see, e.g., Frampton, G. M., et al. (2013) Nat. Biotechnol. 31(11):1023-31. As shown in Table 10, ALK gene fusions involving numerous gene fusion partners were identified.

TABLE 10
ALK gene fusions.

| Fusion | | Number of |
|---|---|---|
| Number | Fusion | Cases Identified |
| 1 | ALK-AGAP1 | 2 |
| 2 | ALK-COL3A1 | 2 |
| 3 | ALK-KIF5C | 1 |
| 4 | ALK-LRRFIP2 | 2 |
| 5 | ALK-MYO5C | 2 |
| 6 | ALK-SRSF7 | 2 |
| 7 | ALK-ABCB11 | 1 |
| 8 | ALK-ACTN4 | 1 |
| 9 | ALK-APH1A | 1 |
| 10 | ALK-AZI2 | 1 |
| 11 | ALK-BTBD9 | 1 |
| 12 | ALK-C2orf73 | 1 |
| 13 | ALK-CAPN14 | 1 |
| 14 | ALK-CARMIL1 | 1 |
| 15 | ALK-CASP8 | 1 |
| 16 | ALK-CDC42BPA | 1 |
| 17 | ALK-CIB4 | 1 |
| 18 | ALK-CNTNAP5 | 1 |
| 19 | ALK-CPQ | 1 |
| 20 | ALK-CPSF7 | 1 |
| 21 | ALK-CREBBP | 1 |
| 22 | ALK-CTBP1 | 1 |
| 23 | ALK-CTNND1 | 1 |
| 24 | ALK-CYP51A1 | 1 |
| 25 | ALK-CYS1 | 1 |
| 26 | ALK-EPHA2 | 1 |
| 27 | ALK-FHOD3 | 1 |
| 28 | ALK-FILIP1L | 1 |
| 29 | ALK-GMCL1 | 1 |
| 30 | ALK-GPN1 | 1 |
| 31 | ALK-GPR113 | 1 |
| 32 | ALK-HADHA | 1 |
| 33 | ALK-HS1BP3 | 1 |
| 34 | ALK-INTS9 | 1 |
| 35 | ALK-ITGA6 | 1 |
| 36 | ALK-KCTD18 | 1 |
| 37 | ALK-KLC4 | 1 |
| 38 | ALK-LINC00535 | 1 |
| 39 | ALK-MAGOHB | 1 |
| 40 | ALK-MAMDC4 | 1 |
| 41 | ALK-MANBA | 1 |
| 42 | ALK-MAP3K9 | 1 |
| 43 | ALK-MED13L | 1 |
| 44 | ALK-METTL25 | 1 |
| 45 | ALK-MTBP | 1 |
| 46 | ALK-MYH10 | 1 |
| 47 | ALK-NFIA | 1 |
| 48 | ALK-NINJ2 | 1 |
| 49 | ALK-OPRM1 | 1 |
| 50 | ALK-OTX1 | 1 |
| 51 | ALK-PAQR4 | 1 |
| 52 | ALK-PDCD10 | 1 |
| 53 | ALK-PDE3A | 1 |
| 54 | ALK-PELI1 | 1 |
| 55 | ALK-PLEC | 1 |
| 56 | ALK-PTGER4 | 1 |
| 57 | ALK-PTPRJ | 1 |
| 58 | ALK-QKI | 1 |
| 59 | ALK-RPS6KA5 | 1 |
| 60 | ALK-SASH1 | 1 |
| 61 | ALK-SEC16B | 1 |
| 62 | ALK-SKAP1 | 1 |
| 63 | ALK-SLC25A13 | 1 |
| 64 | ALK-SLC30A6 | 1 |
| 65 | ALK-SNX17 | 1 |
| 66 | ALK-SOX13 | 1 |
| 67 | ALK-TANGO6 | 1 |
| 68 | ALK-TG | 1 |
| 69 | ALK-TMCO3 | 1 |
| 70 | ALK-TNS3 | 1 |
| 71 | ALK-TRIM24 | 1 |
| 72 | ALK-TTC28 | 1 |
| 73 | ALK-UBE2L3 | 1 |
| 74 | ALK-UBE3B | 1 |
| 75 | ALK-UTRN | 1 |
| 76 | ALK-VASP | 1 |
| 77 | ALK-WDR92 | 1 |
| 78 | ALK-YPEL5 | 1 |
| 79 | ALK-ZNF446 | 1 |
| 80 | ALK-ZNF454 | 1 |
| 81 | ALK-ZNF513 | 1 |
| 82 | ALK-ZSWIM2 | 1 |

As shown in Table 11, detailed characterization of the identified ALK gene fusions revealed that the fusions were present in diverse cancer types, with various fusion partner genes and breakpoints.

TABLE 11
Detailed Characteristics of ALK Gene Fusions.

| Fusion | 5′ breakpoint/ | | Fusion transcript |
|---|---|---|---|
| Exon and breakpoint | 3′ breakpoint/ | | sequence |
| information | 5′ Exons/ | 5′ Refseq ID/ | Fusion protein |
| Tumor type | 3′ Exons/ | 3′ Refseq ID/ | sequence |
| ALK-COL3A1 | chr2: 189873851- | NM_000090/ | SEQ ID NO: 1 |
| 5′-COL3A1(ex1-48 | 18987389/ | NM_004304 | |
| NM_000090)-ALK | chr2: 29448329- | | SEQ ID NO: 44 |
| (ex19-29 NM_004304). | 29448369 | | |
| Breakpoints COL3A1 | COL3A1 ex1-48/ | | |
| intron 48, ALK intron 18. | ALK ex19-29 | | |
| Reciprocal: no | | | |
| Soft tissue sarcoma (NOS) | | | |
| In frame fusion, includes | | | |
| ALK kinase domain. | | | |
| ALK-CDC42BPA | chr1: 227258447- | NM_003607/ | SEQ ID NO: 2 |
| 5′-CDC42BPA(ex1-20 | 227258641/ | NM_004304 | |
| NM_003607)-ALK | chr2: 29447175- | | SEQ ID NO: 45 |
| (ex20-29 NM_004304). | 29447325 | | |
| Breakpoints CDC42BPA | CDC42BPA | | |
| intron 20, ALK intron 19 | ex1-20/ALK | | |
| Ovary serous carcinoma | ex20-29 | | |
| In frame fusion, includes | | | |
| ALK kinase domain. | | | |
| ALK-EPHA2 | chr1: 16473032- | NM_004431/ | SEQ ID NO: 3 |
| 5′-EPHA2(ex1-3 | 16473610/ | NM_004304 | |
| NM_004431)-ALK | chr2: 29449267- | | SEQ ID NO: 46 |
| (ex19-29 NM_004304). | 29449791 | | |
| Breakpoints ALK intron | EPHA2 ex1-3/ | | |
| 18, EPHA2 intron 3. | ALK ex19-29 | | |
| Reciprocal: yes | | | |
| Colon adenocarcinoma | | | |
| (CRC) | | | |
| In frame fusion, includes | | | |
| ALK kinase domain. | | | |
| ALK-MYO5C | chr15: 52513187- | NM_018728/ | SEQ ID NO: 4 |
| 5′-MYO5C (ex1-30 | 52513409/ | NM_004304 | |
| NM_018728)-ALK | chr2:29447042- | | SEQ ID NO: 47 |
| (ex20-29 NM_004304). | 29447276 | | |
| Breakpoints in MYO5C | MYO5C ex1-30/ | | |
| intron 30 and ALK intron | ALK ex20-29 | | |
| 19 | | | |
| Lung adenocarcinoma | | | |
| In frame fusion, includes | | | |
| ALK kinase domain. | | | |
| ALK-TRIM24 | chr7: 138259438- | NM_003852/ | SEQ ID NO: 5 |
| 5′-TRIM24(ex1-12 | 138259669/ | NM_004304 | |
| NM_003852)-ALK | chr2: 29447383- | | SEQ ID NO: 48 |
| (ex20-29 NM_004304). | 29447690 | | |
| Breakpoints TRIM24 | TRIM24 ex1-12/ | | |
| intron 12, ALK intron 19 | ALK ex20-29 | | |
| Lung adenocarcinoma | | | |
| In frame fusion, includes | | | |
| ALK kinase domain. | | | |
| ALK-SKAP1 | chr17: 46426149- | NM_003726/ | SEQ ID NO: 6 |
| 5′-SKAP1(ex1-3 | 46426398/ | NM_004304 | |
| NM_003726)-ALK | chr2: 29446499- | | SEQ ID NO: 49 |
| (ex20-29 NM_004304). | 29446749 | | |
| Breakpoints SKAP1 | SKAP1 ex1-3/ | | |
| intron 3, ALK intron 19 | ALK ex20-29 | | |
| Ovary endometrioid | | | |
| adenocarcinoma | | | |
| In frame fusion, includes | | | |
| ALK kinase domain. | | | |
| ALK-UBE3B | chr12: 109937277- | NM_130466/ | SEQ ID NO: 7 |
| 5′-UBE3B(ex1-11 | 109937406/ | NM_004304 | |
| NM_130466)-ALK | chr2: 29446869- | | SEQ ID NO: 50 |
| (ex20-29 NM_004304). | 29447287 | | |
| Breakpoints UBE3B | UBE3B ex1-11/ | | |
| intron 11, ALK intron 19. | ALK ex20-29 | | |
| Lung adenocarcinoma | | | |
| In frame fusion, includes | | | |
| ALK kinase domain. | | | |
| ALK-TNS3 | chr7: 47333336- | NM_022748/ | SEQ ID NO: 8 |
| 5′-TNS3(ex1-25 | 47333376/ | NM_004304 | |
| NM_022748)-ALK | chr2: 29446279- | | SEQ ID NO: 51 |
| (ex20-29 NM_004304). | 29446319 | | |
| Breakpoints TNS3 intron | TNS3 ex1-25/ | | |
| 25, ALK intron 19. | ALK ex20-29 | | |
| Reciprocal: no | | | |
| Lymph node Castleman’s | | | |
| disease | | | |
| In frame fusion, includes | | | |
| ALK kinase domain. | | | |
| ALK-C2orf73 | chr2: 54571576- | NM_001100396/ | SEQ ID NO: 9 |
| 5′-C2orf73(ex1-3 | 54571912/ | NM_004304 | |
| NM_001100396)-ALK | chr2: 29447055- | | SEQ ID NO: 52 |
| (ex20-29 NM_004304). | 29447357 | | |
| Breakpoints C2orf73 | C2orf73 ex1-3/ | | |
| intron 3, ALK intron 19 | ALK ex20-29 | | |
| Lung adenocarcinoma | | | |
| In frame fusion, includes | | | |
| ALK kinase domain. | | | |
| ALK-AZI2 | chr3: 28367651- | NM_022461/ | SEQ ID NO: 10 |
| 5′-AZI2(ex1-7 | 28368004/ | NM_004304 | |
| NM_022461)-ALK | chr2: 29446143- | | SEQ ID NO: 53 |
| (ex20-29 NM_004304). | 29446537 | | |
| Breakpoints AZI2 intron | AZI2 ex1-7/ | | |
| 7, ALK intron 19. | ALK ex20-29 | | |
| Reciprocal: no | | | |
| Gastroesophageal junction | | | |
| adenocarcinoma | | | |
| In frame fusion, includes | | | |
| ALK kinase domain. | | | |
| ALK-MANBA | chr4: 103560841- | NM_005908/ | SEQ ID NO: 11 |
| 5′-MANBA(ex1-14 | 103561084/ | NM_004304 | |
| NM_005908)-ALK | chr2: 29447666- | | SEQ ID NO: 54 |
| (ex20-29 NM_004304). | 29447971 | | |
| Breakpoints MANBA | MANBA ex1-14/ | | |
| intron 14, ALK intron 19. | ALK ex20-29 | | |
| Lung adenocarcinoma | | | |
| In frame fusion, includes | | | |
| ALK kinase domain. | | | |
| ALK-CNTNAP5 | chr2: 124982770- | NM_130773/ | SEQ ID NO: 12 |
| 5′-CNTNAP(ex1-2 | 124983057/ | NM_004304 | |
| NM_130773)-ALK | chr2: 29447716- | | SEQ ID NO: 55 |
| (ex20-29 NM_004304). | 29448231 | | |
| Breakpoints CNTNAP5 | CNTNAP5 ex1-2/ | | |
| intron 2, ALK intron 19. | ALK ex20-29 | | |
| Reciprocal: no. | | | |
| Lung adenocarcinoma | | | |
| In frame fusion, includes | | | |
| ALK kinase domain. | | | |
| ALK-TANGO6 | chr16: 68877512- | NM_024562/ | SEQ ID NO: 14 |
| 5′-TANGO6(ex1-1 | 68877552 | NM_004304 | |
| NM_024562)-ALK | chr2: 29940445- | | SEQ ID NO: 57 |
| (ex2-29 NM_004304). | 29940485 | | |
| Breakpoints TANGO6 | TANGO6 ex1-1/ | | |
| intron 1, ALK intron 1. | ALK ex2-29 | | |
| Soft tissue sarcoma | | | |
| undifferentiated | | | |
| In frame fusion, includes | | | |
| ALK kinase domain. | | | |
| ALK-NFIA | chr1: 61554202- | NM_005595/ | SEQ ID NO: 15 |
| 5′-NFIA(ex1-2 | 61554242/ | NM_004304 | |
| NM_005595)-ALK | chr2: 29917777- | | SEQ ID NO: 58 |
| (ex2-29 NM_004304). | 29917817 | | |
| Breakpoints NFIA | NFIA ex1-2/ | | |
| intron 2, ALK intron 1. | ALK ex2-29 | | |
| Reciprocal: no. | | | |
| Soft tissue liposarcoma | | | |
| In frame fusion, includes | | | |
| ALK kinase domain. | | | |
| ALK-RPS6KA5 | chr14: 91489071- | NM_004755/ | SEQ ID NO: 16 |
| 5′-RPS6KA5(ex1-1 | 91489199/ | NM_004304 | |
| NM_004755)-ALK | chr2: 29541078- | | SEQ ID NO: 59 |
| (ex8-29 NM_004304). | 29541335 | | |
| Breakpoints RPS6KA5 | RPS6KA5 ex1-1/ | | |
| intron 1, ALK intron 7 | ALK ex8-29 | | |
| Pertoneum serous | | | |
| carcinoma | | | |
| In frame fusion, includes | | | |
| ALK kinase domain. | | | |
| ALK-TG | chr8: 133902377- | NM_003235/ | SEQ ID NO: 17 |
| 5′-TG(ex1-10 | 133902916/ | NM_004304 | |
| NM_003235)-ALK | chr2: 29449335- | | SEQ ID NO: 60 |
| (ex19-29 NM_004304). | 29449926 | | |
| Breakpoints ALK intron | TG ex1-10/ | | |
| 18, TG intron 10. | ALK ex19-29 | | |
| Reciprocal: yes | | | |
| Thyroid papillary | | | |
| carcinoma | | | |
| In frame fusion, includes | | | |
| ALK kinase domain. | | | |
| ALK-LRRFIP2 | chr3: 37098839- | NM_006309/ | SEQ ID NO: 18 |
| 5′-LRRFIP2(ex1-25 | 37099184/ | NM_004304 | |
| NM_006309)-ALK | chr2: 29447444- | | SEQ ID NO: 61 |
| (ex20-29 NM_004304). | 29447873 | | |
| Breakpoints LRRFIP2 | LRRFIP2 ex1-25/ | | |
| intron 25, ALK intron 19 | ALK ex20-29 | | |
| Lung non-small cell | | | |
| lung carcinoma (NOS) | | | |
| In frame fusion, includes | | | |
| ALK kinase domain. | | | |
| ALK-MYO5C | chr15: 52512435- | NM_018728/ | SEQ ID NO: 19 |
| 5′MYO5C(ex1-30 | 52512748/ | NM_004304 | |
| NM_018728)-ALK | chr2: 29447704- | | SEQ ID NO: 62 |
| (ex20-29 NM_004304). | 29448298 | | |
| Breakpoints MYO5C | | | |
| intron 30, ALK intron 19 | | | |
| Lung adenocarcinoma | | | |
| In frame fusion, includes | | | |
| ALK kinase domain. | | | |
| ALK-AGAP1 | chr2: 236691113- | NM_014914/ | SEQ ID NO: 20 |
| 5′-AGAP1(ex1-6 | 236691343/ | NM_004304 | |
| NM_014914)-ALK | chr2: 29449663- | | SEQ ID NO: 63 |
| (ex18-29 NM_004304). | 29449982 | | |
| Breakpoints AGAP1 | AGAP1 ex1-6/ | | |
| intron 6, ALK intron 17 | ALK ex18-29 | | |
| Unknown primary | | | |
| myoepithelial carcinoma | | | |
| In frame fusion, includes | | | |
| ALK kinase domain. | | | |
| ALK-MED13L | chr2: 30142862- | NM_004304/ | SEQ ID NO: 21 |
| 5′-ALK(ex1-1 | 30142902/ | NM_015335 | |
| NM_004304)-MED13L | chr12: 116457697- | | SEQ ID NO: 64 |
| (ex6-31 NM_015335). | 116457737 | | |
| Breakpoints ALK | ALK ex1-1/ | | |
| intron 1, MED13L | MED13L ex6-31 | | |
| intron 5. | | | |
| Reciprocal: no | | | |
| Pediactric soft tissue | | | |
| sarcoma undifferentiated | | | |
| In frame fusion. | | | |
| ALK-MTBP | chr2: 29450415- | NM_004304/ | SEQ ID NO: 22 |
| 5′-ALK(ex1-17 | 29450642/ | NM_022045 | |
| NM_004304)-MTBP | chr8: 121498376- | | SEQ ID NO: 65 |
| (ex12-22 NM_022045). | 121498554 | | |
| Breakpoints ALK | | | |
| intron 17, MTBP intron 11 | | | |
| Ovary serous carcinoma | | | |
| In frame fusion. | | | |
| ALK-SLC30A6 | chr2: 29449705- | NM_004304/ | SEQ ID NO: 24 |
| 5′-ALK(ex1-18 | 29450046/ | NM_017964 | |
| NM_004304)-SLC30A6 | chr2: 32400119- | | SEQ ID NO: 67 |
| (ex4-14 NM_017964). | 32400478 | | |
| Breakpoints ALK | ALK ex1-18/ | | |
| intron 18, SLC30A6 | SLC30A6 ex4-14 | | |
| intron 3 | | | |
| Lung non-small cell lung | | | |
| carcinoma (NOS) | | | |
| In frame fusion. | | | |
| ALK-GMCL1 | chr2: 29420355- | NM_004304/ | SEQ ID NO: 25 |
| 5′-ALK(ex1-27 | 29420535 | NM_178439 | |
| NM_004304)-GMCL1 | chr2: 70072839- | | SEQ ID NO: 68 |
| (ex7-14 NM_178439). | 70073051 | | |
| Breakpoints ALK | ALK ex1-27/ | | |
| intron 27, GMCL1 | GMCL1 ex7-14 | | |
| intron 6 | | | |
| Prostate acinar | | | |
| adenocarcinoma | | | |
| In frame fusion. | | | |
| AKA-AGAP1 | chr2: 29447436- | NM_004304/ | SEQ ID NO: 26 |
| 5′-ALK(ex1-19 | 29447953/ | NM_014914 | |
| NM_004304)-AGAP1 | chr2: 236681905- | | SEQ ID NO: 69 |
| (ex7-17 NM_014914). | 236682235 | | |
| Breakpoints ALK | ALK ex1-19/ | | |
| intron 19, AGAP1 intron 6. | AGAP1 ex7-17 | | |
| Lung adenocarcinoma | | | |
| In frame fusion. | | | |
| ALK-ZNF454 | chr2: 29446550- | NM_004304/ | SEQ ID NO: 27 |
| 5′-ALK(ex1-19 | 29446728/ | NM_182594 | |
| NM_004304)-ZNF454 | chr5: 17380665- | | SEQ ID NO: 70 |
| (ex5-5 NM_182594). | 178380839 | | |
| Breakpoints ALK intron | ALK ex1-19/ | | |
| 19, AGAP1 intron 6. | ZNF454 ex5-5 | | |
| Lung adenocarcinoma | | | |
| In frame fusion. | | | |
| ALK-TTC28 | chr22: 29070004- | NM_001145418/ | SEQ ID NO: 28 |
| 5′-TTC28(ex1-1 | 29070137/ | NM_004304 | |
| NM_001145418)-ALK | chr2: 29456365- | | SEQ ID NO: 71 |
| (ex14-299 NM_004304). | 29456651 | | |
| Breakpoints TTC28 | TTC28 ex1-1/ | | |
| intron 1, ALK intron 13. | ALK ex14-29 | | |
| Reciprocal no | | | |
| Uterus carcinosarcoma | | | |
| In frame fusion, includes | | | |
| ALK kinase domain. | | | |
| ALK-NINJ2 | chr12: 758744- | NM_016533/ | SEQ ID NO: 29 |
| 5′-NINJ2(ex1-1 | 759024/ | NM_004304 | |
| NM_016533)-ALK | chr2: 29456325- | | SEQ ID NO: 72 |
| (ex14-29 NM_004304). | 29456573 | | |
| Breakpoints NINJ2 | NINJ2 ex1-1/ | | |
| intron 1, ALK intron 13 | ALK ex14-29 | | |
| Breast invasive ductal | | | |
| carcinoma (IDC) | | | |
| In frame fusion, includes | | | |
| ALK kinase domain. | | | |
| ALK-UTRN | chr6: 145163819- | NM_007124/ | SEQ ID NO: 30 |
| 5′-UTRN(ex1-72 | 145164312/ | NM_004304 | |
| NM_007124)-ALK | chr2: 29448627- | | SEQ ID NO: 73 |
| (ex19-29 NM_004304). | 29449031 | | |
| Breakpoints UTRN | UTRN ex1-72/ | | |
| intron 72, ALK intron 18 | ALK ex19-29 | | |
| Pancreas ductal | | | |
| adenocarcinoma | | | |
| In frame fusion, includes | | | |
| ALK kinase domain. | | | |
| ALK-ACTN4 | chr19: 39200190- | NM_004924/ | SEQ ID NO: 31 |
| 5′-ACTN4(ex1-7 | 39200558 | NM_004304 | |
| NM_004924)-ALK | vhr2: 29446977- | | SEQ ID NO: 74 |
| (ex20-29 NM_004304). | 29447411 | | |
| Breakpoints ACTN4 | ACTN4 ex1-7/ | | |
| intron 7, ALK intron 19 | ALK ex20-29 | | |
| Lung adenocarcinoma | | | |
| In frame fusion, includes | | | |
| ALK kinase domain. | | | |
| ALK-CPSF7 | chr11: 61186432- | NM_024811/ | SEQ ID NO: 32 |
| 5′-CPSF7 (ex1-5 | 61186683/ | NM_004304 | |
| NM_024811)-ALK | chr2: 29447932- | | SEQ ID NO: 75 |
| (ex20-29). | 29448250 | | |
| Breakpoints in CPSF7 | CPSF7 ex1-5/ | | |
| intron 5 and ALK | ALK ex20-29 | | |
| intron 19. | | | |
| In frame fusion, includes | | | |
| ALK kinase domain. | | | |
| ALK-SLC25A13 | chr7: 95814076- | NM_014251/ | SEQ ID NO: 33 |
| 5′-ALC25A13(ex1-10 | 95814403/ | NM_004304 | |
| NM_014251)-ALK | chr2: 29446496- | | SEQ ID NO: 76 |
| (ex20-29 NM_004304). | 29446887 | | |
| Breakpoints SLC25A13 | SLC25A13 ex1-10/ | | |
| intron 10, ALK intron 19 | ALK ex20-29 | | |
| Lung adenocarcinoma | | | |
| In fusion frame, includes | | | |
| ALK kinase domain. | | | |
| ALK-CTNND1 | chr11: 57580664- | NM_001331/ | SEQ ID NO: 34 |
| 5′-CTNND1(ex1-16 | 57580962/ | NM_004304 | |
| NM_001331)-ALK | chr2: 29446968- | | SEQ ID NO: 77 |
| (ex20-x29 NM_004304). | 29447253 | | |
| Breakpoints CTNND1 | CTNND1 ex1-16/ | | |
| intron 16 and ALK | ALK ex20-29 | | |
| intron 19. | | | |
| Reciprocal: no | | | |
| Lung adenocarcinoma | | | |
| In frame fusion, includes | | | |
| ALK kinase domain. | | | |
| ALK-KLC4 | chr6: 43039012- | NM_201521/ | SEQ ID NO: 35 |
| 5′-KLC4(ex1-9 | 43039212/ | NM_004304 | |
| NM_201521)-ALK | chr2: 29447007- | | SEQ ID NO: 78 |
| (ex20-29 NM_004304). | 29447235 | | |
| Breakpoints KLC4 | KLC4 ex1-9/ | | |
| intron 9, ALK intron 19. | ALK ex20-29 | | |
| Lung adenocarcinoma | | | |
| In frame fusion, includes | | | |
| ALK kinase domain. | | | |
| ALK-BTBD9 | chr6: 38549536- | NM_052893/ | SEQ ID NO: 36 |
| 5′-BTBD9(ex1-5 | 38549796/ | NM_004304 | |
| NM_052893)-ALK | chr2: 29447685- | | SEQ ID NO: 79 |
| (ex20-29 NM_004304). | 29447981 | | |
| Breakpoints BTBD9 | BTBD9 ex1-5/ | | |
| intron 5, ALK intron 19. | ALK ex20-29 | | |
| Lung non-small cell | | | |
| lung carcinoma (NOS) | | | |
| In frame fusion, includes | | | |
| ALK kinase domain. | | | |
| ALK-CPQ | chr8: 98095364- | NM_016134/ | SEQ ID NO: 38 |
| 5′-CPQ(ex1-7 | 98095503/ | NM_004304 | |
| NM_016134)-ALK | chr2: 29447780- | | SEQ ID NO: 81 |
| (ex20-29 NM_004304). | 29448006 | | |
| Breakpoints in CPQ | CPQ ex1-7/ | | |
| intron 7 and ALK intron 19 | ALK ex20-29 | | |
| Lung non-small cell lung | | | |
| carcinoma (NOS) | | | |
| In frame fusion, includes | | | |
| ALK kinase domain. | | | |
| ALK-KIF5C | chr8: 149821504- | NM_016134/ | SEQ ID NO: 38 |
| 5′-CPQ(ex1-7 | 98095503/ | NM_004304 | |
| NM_016134)-ALK | chr2: 29447780- | | SEQ ID NO: 81 |
| (ex20-29 NM_004304). | 29448006 | | |
| Breakpoints in CPQ | CPQ ex1-7/ | | |
| intron 7 and ALK intron 19 | ALK ex20-29 | | |
| Lung non-small cell lung | | | |
| carcinoma (NOS) | | | |
| In frame fusion, includes | | | |
| ALK kinase domain. | | | |
| ALK-KIF5C | chr2: 149821504- | NM_004522/ | SEQ ID NO: 40 |
| 5′-KIF5C(ex1-11 | 149821810/ | NM_004304 | |
| NM_004522)-ALK | chr2: 29446775- | | SEQ ID NO: 83 |
| (ex20-29 NM_004304). | 29447077 | | |
| Breakpoints KIF5C | KIF5C ex1-11/ | | |
| intron 11, ALK intron 19 | ALK ex20-29 | | |
| Unknown primary | | | |
| carcinoma (NOS) | | | |
| In frame fusion, includes | | | |
| ALK kinase domain. | | | |
| ALK-MAGOHB | chr12: 10765965- | NM_018048/ | SEQ ID NO: 41 |
| 5′-MAGOHB(ex1-1 | 10765965/ | NM_004304 | |
| NM_018048)-ALK | chr2: 29446915- | | SEQ ID NO: 84 |
| (ex20-29 NM_004304). | 29446915 | | |
| Breakpoints MAGOHB | MAGOHB ex1-1/ | | |
| intron 1, ALK intron 19 | ALK ex20-29 | | |
| Anus squamous cell | | | |
| carcinoma | | | |
| In frame fusion, includes | | | |
| ALK kinase domain. | | | |
| ALK-COL3A1 | chr2: 189873873- | NM_000090/ | SEQ ID NO: 43 |
| 5′-COL3A1(ex1-48 | 189873913/ | NM_004304 | |
| NM_000090)-ALK | chr2: 29448334- | | SEQ ID NO: 86 |
| (ex19-29 NM_004304). | 29448374 | | |
| Breakpoints COL3A1 | COL3A1 ex1-48/ | | |
| intron 48, ALK intron 18. | ALK ex19-29 | | |
| Reciprocal: no | | | |
| Uterus leiolyosarcoma | | | |
| In frame fusion, includes | | | |
| ALK kinase domain. | | | |
| ALK-OPRM1 | chr6: 154466451- | NM_001008503/ | |
| 5′-OPRM1(ex1-3 | 154466754/ | NM_004304 | |
| NM_001008503)-ALK | chr2: 29446794- | | |
| (ex20-29 NM_004304). | 29447151 | | |
| Breakpoints OPRM1 | OPRM1 ex1-3/ | | |
| intron 3, ALK intron 19. | ALK ex20-29 | | |
| Lung adenocarcinoma | | | |
| Fusion includes ALK | | | |
| kinase domain. | | | |
| ALK-GPN1 | chr2: 27851952- | NM_007266/ | |
| 5′-GPN1(ex1-1 | 27852701/ | NM_004304 | |
| NM_007266)-ALK | chr2: 29451600- | | |
| (ex16-29 NM_004304). | 29452075 | | |
| Breakpoints GPN1 | GPN1 ex1-1/ | | |
| intron 1, ALK intron 15. | ALK ex16-29 | | |
| Reciprocal: yes | | | |
| Skin melanoma | | | |
| Fusion includes ALK | | | |
| kinase domain. | | | |
| ALK-SEC16B | chr1: 177916828- | NM_033127/ | |
| 5′-SEC16B(ex1-13 | 177917021/ | NM_004304 | |
| NM_033127)-ALK | chr2: 29456427- | | |
| (ex14-29 NM_004304). | 29456627 | | |
| Breakpoints SEC16B | SEC16B ex1-13/ | | |
| intron 13, ALK intron 13 | ALK ex14-29 | | |
| Esophagus adenocarcinoma | | | |
| Fusion includes ALK | | | |
| kinase domain. | | | |
| ALK-UBE2L3 | chr22: 21931222- | NM_00347/ | |
| 5′-UBE2L3(ex1-1 | 21931915/ | NM_004304 | |
| NM_00347)-ALK | chr2: 29450281- | | |
| (ex18-29 NM_004304). | 29450692 | | |
| Breakpoints ALK intron | UBE2L3 ex1-1/ | | |
| 17, UBE2L3 intron 1; | ALK ex18-29 | | |
| Reciprocal: Yes | | | |
| Skin melanoma | | | |
| Fusion includes ALK | | | |
| kinase domain. | | | |
| ALK-METTL25 | chr12: 82843057- | NM_032230/ | |
| 5′-METTL25(ex1-8 | 82843199/ | NM_004304 | |
| NM_032230 )-ALK | chr2: 29446633- | | |
| (ex20-29 NM_004304). | 29446761 | | |
| Breakpoints in ALK | METTL25 ex1-8/ | | |
| intron 19 and METTL25 | ALK ex20-29 | | |
| intron 8 | | | |
| Lung adenocarcinoma | | | |
| Fusion includes ALK | | | |
| kinase domain. | | | |
| ALK-CYS1 | chr2: 10214734- | NM_001037160/ | |
| 5′-CYS1(ex1-1 | 10215222/ | NM_004304 | |
| NM_001037160)-ALK | chr2: 29447165- | | |
| (NM_004304). | 29447594 | | |
| Breakpoints CYS1 | CYS1 ex1-1/ | | |
| intron 1, ALK intron 19 | ALK ex20-29 | | |
| Lung adenocarcinoma | | | |
| Fusion includes ALK | | | |
| kinase domain. | | | |
| ALK-ABCB11 | chr2: 169782189- | NM_003742/ | |
| 5′-ABCB11 (ex1-26 | 169782398/ | NM_004304 | |
| NM_003742)-ALK | chr2: 29447182- | | |
| (ex20-29 NM_004304). | 29447532 | | |
| Breakpoints in ABCB11 | ABCB11 ex1-26/ | | |
| intron 26 and ALK | ALK ex20-29 | | |
| intron 19 | | | |
| Lung non-small cell lung | | | |
| carcinoma (NOS) | | | |
| Fusion includes ALK | | | |
| kinase domain. | | | |
| ALK-INTS9 | chr8: 28655333- | NM_018250/ | |
| 5′-INTS9(ex1-8 | 28655587/ | NM_004304 | |
| NM_018250)-ALK | chr2: 29447949- | | |
| (ex20-29 NM_004304). | 29448294 | | |
| Breakpoints INTS9 | INTS ex1-8/ | | |
| intron 8 and ALK intron 19. | ALK ex20-29 | | |
| Reciprocal: No | | | |
| Lung adenocarcinoma | | | |
| Fusion includes ALK | | | |
| kinase domain. | | | |
| ALK-CIB4 | chr2: 26842893- | NM_001029881/ | |
| 5′-CIB4(ex1-3 | 26843172/ | NM_004304 | |
| NM_001029881)-ALK | chr2: 29448034- | | |
| (ex20-29 NM_004304). | 29448163 | | |
| Breakpoints CIB4 intron 3, | CIB ex1-3/ | | |
| ALK intron 19. | ALK ex20-29 | | |
| Lung adenocarcinoma | | | |
| Fusion includes ALK | | | |
| kinase domain. | | | |
| ALK-WDR92 | chr2: 68361884- | NM_138458/ | |
| 5′WDR92(ex1-7 | 68361924/ | NM_004304 | |
| NM_138458)-ALK | chr2: 29917846- | | |
| (ex2-29 NM_004304). | 29917886 | | |
| Breakpoints WDR92 | WDR92 ex1-7/ | | |
| intron 7, ALK intron 1. | ALK ex2-29 | | |
| Reciprocal: no | | | |
| Soft tissue leiomyosarcoma | | | |
| Fusion includes ALK | | | |
| kinase domain. | | | |
| ALK-OTX1 | chr2: 29446763- | NM_004304/ | |
| 5′-ALK(ex1-19 | 29447082/ | NM_014562 | |
| NM_004304)-OTX1 | chr2: 63282360- | | |
| (ex5-5 NM_014562). | 63282649 | | |
| Breakpoints ALK intron | ALK ex1-19/ | | |
| 19, OTX1 intron 4. | OTX1 ex5-5 | | |
| Lung adenocarcinoma | | | |
| ALK-PDCD10 | chr2: 29455118- | NM_004304/ | |
| 5′-ALK(ex1-15 | 29455261 | NM_007217 | |
| NM_004304)-PDCD10 | chr3: 167443055- | | |
| (ex3-9 NM_007217). | 167443292 | | |
| Breakpoints ALK intron 15, | ALK ex1-15/ | | |
| PDCD10 intron 2 | PDCD10 ex3-9 | | |
| Breast carcinoma (NOS) | | | |
| ALK-PTGER4 | chr2: 29446495- | NM_004304/ | |
| 5′-ALK(ex1-19 | 29446590/ | NM_000958 | |
| NM_004304)-PTGER | chr5: 40688492- | | |
| (ex3-3 NM_000958). | 40688617 | | |
| Breakpoints ALK intron 19, | ALK ex1-19/ | | |
| PTGER4 intron 2 | PTGER4 ex3-3 | | |
| Prostate acinar | | | |
| adenocarcinoma | | | |
| ALK-PTPRJ | chr2: 29447003- | NM_004304/ | |
| 5′-ALK(ex1-19 | 29447320/ | NM_002843 | |
| NM_004304)-PTPRJ | chr11: 48037100- | | |
| (ex2-25 NM_002843). | 48037371 | | |
| Breakpoints ALK intron | ALK ex1-19/ | | |
| 19, PTPRJ intron 1. | PTPRJ ex2-25 | | |
| Lung non-small cell lung | | | |
| carcinoma (NSCLC) (NOS) | | | |
| ALK-ZSWIM2 | chr2: 29447843- | NM_004304/ | |
| 5′-ALK(ex1-19 | 29448180/ | NM_182521 | |
| NM_182521). | chr2: 187699208- | | |
| Breakpoints ALK intron 19, | 187699458 | | |
| ZSWIM2 intron 5. | ALK ex1-19/ | | |
| Lung adenocarcinoma | ZSWIM2 ex6-9 | | |
| ALK-FHOD3 | chr2: 29449375- | NM_004304/ | |
| 5′-ALK(ex1-18 | 29449493/ | NM_025135 | |
| NM_004304)-FHOD3 | chr18: 34195667- | | |
| (ex10-25 NM_025135). | 34195872 | | |
| Breakpoints ALK intron 18, | ALK ex1-18/ | | |
| FHOD3 intron 9 | FHOD3 ex10-25 | | |
| Lung non-small cell lung | | | |
| carcinoma (NSCLC) (NOS) | | | |
| ALK-FILIP1L | chr2: 29449660- | NM_004304/ | |
| 5′-ALK(1-18 | 29449973/ | NM_182909 | |
| NM_004304)-FILP1L | chr3: 99688248- | | |
| (ex2-6 NM_182909). | 99688442 | | |
| Breakpoints ALK intron | ALK ex1-18/ | | |
| 18 and FILIP1L intron 1. | FILP1L ex2-6 | | |
| Reciprocal no | | | |
| Pancreas ductal | | | |
| adenocarcinoma | | | |
| ALK-ITGA6 | chr2: 29447349- | NM_004304/ | |
| 5′-ALK(ex1-19 | 29447349- | NM_000210 | |
| NM_004304)-ITGA6 | chr2: 173326199- | | |
| (ex2-26 NM_000210). | 173326199 | | |
| Breakpoints ALK intron | ALK ex1-19/ | | |
| 19, ITGA6 intron 1 | ITAG6 ex2-26 | | |
| Pancreas cancer (NOS) | | | |
| ALK-KCTD18 | chr2: 29447134- | NM_004304/ | |
| 5′-ALK(ex1-19 | 29447442/ | NM_152387 | |
| NM_004304)-KCTD18 | chr2: 201373509- | | |
| (ex2-7 NM_152387). | 201373886 | | |
| Breakpoints ALK intron 19, | ALK ex1-19/ | | |
| KCTD18 intron 1 | KCTD18 ex2-7 | | |
| Lung adenocarcinoma | | | |
| ALK-MAMDC4 | chr2: 29446201- | NM_004304/ | |
| 5′-ALK(ex1-20 | 29446434/ | NM_206920 | |
| NM_004304)-MAMDC | chr9: 139751298- | | |
| (ec16-27 NM_206920). | 139751629 | | |
| Breakpoints ALK intron | ALK ex1-20/ | | |
| 20, MAMDC4 intron 15. | MAMCD4 ex16-27 | | |
| Ovary clear cell carcinoma | | | |
| ALK-PELI1 | chr2: 64360629- | NM_020651/ | |
| 5′-PELI1(ex1 NM_020651)- | 64360629/ | NM_004304 | |
| ALK(ex20-29 NM_004304). | chr2: 29448116- | | |
| Breakpoints PELI1 intron 1, | 29448116 | | |
| ALK intron 19 | PELI1 ex1/ | | |
| Breast cancer (NOS) | ALK ex20-29 | | |
| Fusion includes ALK | | | |
| kinase domain. | | | |
| PELI exon 1 includes only | | | |
| part of 5′UTR, no coding | | | |
| sequence. | | | |
| ALK-LINC00535 | chr8: 94669651- | NR_033858/ | |
| 5′-LINC00535(ex1-5 | 94669972/ | NM_004304 | |
| NR_033858)-ALK | chr2: 29447755- | | |
| (ex20-29 NM_004304). | 29448172 | | |
| Breakpoints LINC00535 | LINC00535 ex1-5/ | | |
| intron 5, ALK intron 19 | ALK ex20-29 | | |
| Lung non-small cell lung | | | |
| carcinoma (NOS) | | | |
| Fusion includes ALK | | | |
| kinase domain. | | | |
| LINC00535 exons 1-5 | | | |
| include only UTR, no | | | |
| coding sequence. | | | |
| LINC00535 is a non-protein | | | |
| coding gene. | | | |
| ALK-CTBP1 | chr4: 1206164- | NM_001328/ | |
| 5′-CTBP1(ex1-9 | 1206291/ | NM_004304 | |
| NM_001328)-ALK | chr2: 29447285- | | |
| (ex20-29 NM_004304). | 29447572 | | |
| Breakpoints CTBP1 exon 9, | CTBP1 ex1-9/ | | |
| ALK intron 19 | ALK ex20-29 | | |
| Lung adenocarcinoma | | | |
| Fusion includes ALK | | | |
| kinase domain. | | | |
| ALK-CARMIL1 | chr6: 25550917- | NM_017640/ | |
| 5′-CARMIL1(ex1-27 | 25551280/ | NM_004304 | |
| NM_017640)-ALK | chr2: 29447579- | | |
| (ex20-29 NM_004304). | 29447824 | | |
| Breakpoints CARMIL1 | CARMIL1 ex1-27/ | | |
| exon 27, ALK intron 19 | ALK ex20-29 | | |
| Lung adenocarcinoma | | | |
| Fusion includes ALK | | | |
| kinase domain. | | | |
| ALK-ZNF513 | chr2: 27603440- | NM_144631/ | |
| 5′-ZNF513(ex1-1 | 27603620/ | NM_004304 | |
| NM_144631)-ALK | chr2: 30143106- | | |
| (ex1-29 NM_004304). | 30143448 | | |
| Breakpoints ZNF513 | ZNF513 ex1-1/ | | |
| exon 1, ALK exon 1 | ALK ex1-29 | | |
| Gallbladder adenocarcinoma | | | |
| Fusion includes ALK | | | |
| kinase domain. | | | |
| ALK-TMCO3 | chr13: 114163479- | NM_017905/ | |
| 5′-TMCO3(ex1-6 | 114163962/ | NM_004304 | |
| NM_017905)-ALK | chr2: 29497970- | | |
| (ex11-29 NM_004304). | 29498226 | | |
| Breakpoints ALK exon 11, | TMCO3 ex1-6/ | | |
| TMCO3 intron 6 | ALK ex11-29 | | |
| Unknown primary | | | |
| adenocarcinoma | | | |
| Fusion includes ALK | | | |
| kinase domain. | | | |
| ALK-SRSF7 | chr2: 38975362- | NM_001031684/ | |
| 5′-SRSF7(ex1-4 | 38975710 | NM_004304 | |
| NM_0010613684))-ALK | chr2: 29449531- | | |
| (ex18-29 NM_004304). | 29449920 | | |
| Breakpoints SRSF7 intron 4, | SRSF7 ex1-4/ | | |
| ALK exon 18 | ALK ex18-29 | | |
| Colon adenocarcinoma | | | |
| (CRC) | | | |
| Fusion includes ALK | | | |
| kinase domain. | | | |
| ALK-CASP8 | chr2: 202151038- | NM_001228/ | |
| 5′-CASP8(ex1-10 | 202151249/ | NM_004304 | |
| NM_001228)-ALK | chr2: 29969128- | | |
| (ex2-29 NM_004304). | 29969572 | | |
| Breakpoints CASP8 exon | CASP8 ex1-10/ | | |
| 10, ALK intron 1 | ALK ex2-29 | | |
| Vulva squamous cell | | | |
| carcinoma (SCC) | | | |
| Fusion includes ALK | | | |
| kinase domain. | | | |
| ALK-CYP51A1 | chr7: 91745465- | NM_000786/ | |
| 5′-CYP51A1(ex1-9 | 91745693/ | NM_004304 | |
| NM_000786)-ALK | chr2: 29917599- | | |
| (ex3-29 NM_004304). | 29917771 | | |
| Breakpoints CYP51A1 | CYP51A1 ex1-9/ | | |
| intron 9, ALK exon 3 | ALK ex3-29 | | |
| Ovary high grade serous | | | |
| carcinoma | | | |
| Fusion includes ALK | | | |
| kinase domain. | | | |
| ALK-GPR113 | chr2: 26546664- | NM_153835/ | |
| 5′-GPR113(ex1-1 | 26546664/ | NM_004304 | |
| NM_153835)-ALK | chr2: 29448344- | | |
| (ex19-29 NM_004304). | 29448344 | | |
| Breakpoints GPR113 | GPR113 ex1-1/ | | |
| intron 1, ALK exon 19 | ALK ex19-29 | | |
| Prostate acinar | | | |
| adenocarcinoma | | | |
| Fusion includes ALK | | | |
| kinase domain. | | | |
| ALK-HADHA | chr2: 26456000- | NM_000182/ | |
| 5′-HADHA(ex1-5 | 26456242/ | NM_004304 | |
| NM_000182)-ALK | chr2: 29606503- | | |
| (ex5-29 NM_004304). | 29606657 | | |
| Breakpoints HADHA | HADHA ex1-5/ | | |
| intron 5, ALK exon 5 | ALK ex5-29 | | |
| Fallopian tube serous | | | |
| carcinoma | | | |
| Fusion includes ALK | | | |
| kinase domain. | | | |
| ALK-LRRFIP2 | chr3: 37111067- | NM_006309/ | |
| 5′-LRRFIP2(ex1-22 | 37111382/ | NM_004304 | |
| NM_006309)-ALK | chr2: 29446062- | | |
| (ex20-29 NM_004304). | 29446344 | | |
| Breakpoints LRRFIP2 | LRRFIP2 ex1-22/ | | |
| intron 22, ALK exon 20. | ALK ex20-29 | | |
| Lung adenocarcinoma | | | |
| Fusion includes ALK | | | |
| kinase domain. | | | |
| ALK-MYH10 | chr17: 8383515- | NM_005964/ | |
| 5′-MYH10(ex1-38 | 8383800/ | NM_004304 | |
| NM_005964)-ALK | chr2: 29446135- | | |
| (ex20-29 NM_004304). | 29446423 | | |
| Breakpoints MYH10 | MYH10 ex1-38/ | | |
| exon 38, ALK intron 19 | ALK ex20-29 | | |
| Pediactric Soft tissue | | | |
| sarcoma (NOS) | | | |
| Fusion includes ALK | | | |
| kinase domain. | | | |
| ALK-PDE3A | chr12: 20796733- | NM_000921/ | |
| 5′-PDE3A(ex1-10 | 20796837/ | NM_004304 | |
| NM_000921)-ALK | chr2: 29541064- | | |
| (ex8-29 NM_004304). | 29541218 | | |
| Breakpoints PDE3A | PDE3A ex1-10/ | | |
| introno 10, ALK exon 8 | ALK ex8-29 | | |
| Pediatric Bone | | | |
| osteosarcoma | | | |
| Fusion includes ALK | | | |
| kinase domain. | | | |
| ALK-PLEC | chr8: 145009233- | NM_000445/ | |
| 5′-PLEC(ex1-9 | 145009424/ | NM_004304 | |
| NM_000445)-ALK | chr2: 29447974- | | |
| (ex20-29 NM_004304). | 29448217 | | |
| Breakpoints PLEC exon 9, | PLEC ex1-9/ | | |
| ALK intron 19 | ALK ex20-29 | | |
| Uterus endometrial | | | |
| adenocarcinoma | | | |
| endometrioid | | | |
| Fusion includes ALK | | | |
| kinase domain. | | | |
| ALK-QKI | chr6: 163890860- | NM_006775/ | |
| 5′-QKI(ex1-2 | 163891168/ | NM_004304 | |
| NM_006775)-ALK | chr2: 29446065- | | |
| (ex20-29 NM_004304). | 29446378 | | |
| Breakpoints QKI intron 2, | QKI ex1-2/ | | |
| ALK exon 20 | ALK ex20-29 | | |
| Brain glioblastoma (GBM) | | | |
| Fusion includes ALK | | | |
| kinase domain. | | | |
| ALK-SASH1 | chr6: 148853694- | NM_015278/ | |
| 5′-SASH1(ex1-14 | 148853955/ | NM_004304 | |
| NM_015278)-ALK | chr2: 29449754- | | |
| (ex18-29 NM_004304). | 29449930 | | |
| Breakpoint SASH1 exon 14, | SASH1 ex1-14/ | | |
| ALK intron 17 | ALK ex18-29 | | |
| Brain glioblastoma (GBM) | | | |
| Fusion includes ALK | | | |
| kinase domain. | | | |
| ALK-SRSF7 | chr2: 38974723- | NM_001031684/ | |
| 5′-SRSF7(ex1-5 | 38975203/ | NM_004304 | |
| NM_001031684)-ALK | chr2: 29449640- | | |
| (ex18-29 NM_004304). | 29449923 | | |
| Breakpoints SRSF7 intron 5, | SRSF7 ex1-5/ | | |
| ALK exon 18. | ALK ex18-29 | | |
| Pancreas ductal | | | |
| adenocarcinoma | | | |
| Fusion includes ALK | | | |
| kinase domain. | | | |
| ALK-VASP | chr19: 46024804- | NM_003370/ | |
| 5′-VASP(ex1-4 | 46025190/ | NM_004304 | |
| NM_003370)-ALK | chr2: 29543503- | | |
| (ex7-29 NM_004304). | 29543740 | | |
| Breakpoints VASP intron 4, | VASP ex1-4/ | | |
| ALK exon 7 | ALK ex7-29 | | |
| Ovary clear cell carcinoma | | | |
| Fusion includes ALK | | | |
| kinase domain. | | | |
| ALK-ZNF446 | chr19: 58991381- | NM_017908/ | |
| 5′-ZNF446(ex1-7 | 58991664/ | NM_004304 | |
| NM_017908)-ALK | chr2: 29446677- | | |
| (ex20-29 NM_004304). | 29446960 | | |
| Breakpoints ZNF446 | ZNF446 ex1-7/ | | |
| exon 7, ALK intron 19 | ALK ex20-29 | | |
| Ovary serous carcinoma | | | |
| Fusion includes ALK | | | |
| kinase domain. | | | |
| ALK-SOX13 | chr1: 204042405- | NM_005686/ | |
| 5′-SOX13(ex 1 | 204042653/ | NM_004304 | |
| NM_005686)-ALK | chr2: 29449452- | | |
| (ex18-29 NM_004304). | 29449864 | | |
| Breakpoints SOX13 | SOX13 ex1/ | | |
| exon 1, ALK exon 18 | ALK ex18-29 | | |
| Pediatric Skin melanoma | | | |
| Fusion includes ALK | | | |
| kinase domain. | | | |
| SOX13 exon 1 includes | | | |
| only part of 5′UTR, | | | |
| no coding sequence. | | | |
| ALK-YPEL5 | chr2: 20378123- | NM_016061/ | |
| 5′ (YPEL5 ex 1 | 30378424 | NM_004304 | |
| NM_016061)-ALK | chr2: 29449645- | | |
| (ex18-29 NM_004304). | 29449918 | | |
| Breakpoints YPEL5 | YPEL5 ex1/ | | |
| intron 1 and ALK exon 18. | ALKex18-29 | | |
| Reciprocal: no. | | | |
| Fusion includes ALK | | | |
| kinase domain. | | | |
| YPEL5 exon 1 includes | | | |
| only part of 5′UTR, no | | | |
| coding sequence. | | | |
| ALK-CAPN14 | chr2: 29456468- | NM_004304/ | |
| 5′-ALK(ex1-14 | 29456549/ | NM_001145122 | |
| NM_004304)-CAPN14 | chr2: 31426279- | | |
| (ex3-22 NM_001145122). | 31426378 | | |
| Breakpoints ALK exon 14, | ALK ex1-14/ | | |
| CAPN14 intron 2 | CAPN14 ex3-22 | | |
| Small intestine | | | |
| adenocarcinoma | | | |
| ALK-MAP3J9 | chr2: 30142828- | NM_004304/ | |
| 5′-ALK(ex1-1 | 30143036/ | NM_033141 | |
| NM_004304)-MAP3K9 | chr14: 71216554- | | |
| (ex4-13 NM_033141). | 71216698 | | |
| Breakpoints ALK intron 1, | ALK ex1-1/ | | |
| MAP3K9 exon 4 | MAP3K9 ex4-13 | | |
| Breast carcinoma (NOS) | | | |
| ALK-SNX17 | chr2: 29462409- | NM_004304/ | |
| 5′-ALK(ex1-13 | 29462706/ | NM_014748 | |
| NM_004304)-SNX17 | chr2: 27595576- | | |
| (ex3-15 NM_014748). | 27595761 | | |
| Breakpoints ALK intron 13, | ALK ex1-13/ | | |
| SNX17 exon 3 | SNX17 ex3-15 | | |
| Prostate acinar | | | |
| adenocarcinoma | | | |
| ALK-HS1BP3 | chr2: 29446985- | NM_004304/ | |
| 5′-ALK(ex1-19 | 29447221/ | NM_022460 | |
| NM_004304)-HS1BP3 | chr2: 20838147- | | |
| (ex4-7 NM_022460). | 20838335 | | |
| Breakpoints in ALK | ALK ex1-19/ | | |
| intron 19 and HS1BP3 | HS1BP3 ex4-7 | | |
| exon 4. | | | |
| Lung adenocarcinoma | | | |
| ALK-CREBBP | chr2: 29797462- | NM_004304/ | |
| 5′-ALK(ex1-3 | 29797842/ | NM_004380 | |
| NM_004304)-CREBBP | chr16: 3900331- | | |
| (ex2-31 NM_004380). | 3900680 | | |
| Breakpoints ALK intron 3, | ALK ex1-3/ | | |
| CREBBP exon 2 | BREBBP ex2-31 | | |
| Ovary serous carcinoma | | | |
| ALK-PAQR4 | chr2: 29917567- | NM_004304/ | |
| 5′-ALK(ex1-3 | 299178551/ | NM_152341 | |
| NM_004304)-PAQR | chr16: 3019620- | | |
| (ex1-13 NM_152341). | 3019700 | | |
| Breakpoints ALK intron 3, | ALK ex1-3/ | | |
| PAQR4 exon 1 | PAQR4 ex1-3 | | |
| Breast carcinoma (NOS) | | | |
| ALK-APH1A | chr2: 29669577- | NM_004304/ | |
| 5′-ALK(ex1-4 | 29669873/ | NM_016022 | |
| NM_004304)-APH1A | chr1: 150239704- | | |
| (ex4-6 NM_016022). | 150239811 | | |
| Breakpoints ALK intron 4, | ALK ex1-4/ | | |
| APH1A exon 4 | APH1A ex4-6 | | |
| Uterus leiomyosarcoma | | | |

ALK rearrangements, such as the ALK gene fusions identified in this Example, may confer sensitivity of cancers to treatments, such as ALK-targeted therapies. Such ALK-targeted therapies include ALK inhibitors, such as alectinib, brigatinib, ceritinib, crizotinib, entrectinib, and lorlatinib (see, e.g., Peters et al., The New England journal of medicine vol. 377, 9 (2017): 829-838; Gettinger et al., The Lancet. Oncology vol. 17, 12 (2016): 1683-1696; Camidge et al., Journal of clinical oncology, vol. 36, 26 (2018): 2693-2701; Kim et al., The Lancet. Oncology vol. 17, 4 (2016): 452-463; Solomon et al., Journal of clinical oncology, vol. 36, 22 (2018): 2251-2258; Solomon et al., The New England journal of medicine, vol. 371, 23 (2014): 2167-77; Drilon et al., Cancer discovery vol. 7, 4 (2017): 400-409; Shaw et al., The Lancet. Oncology vol. 18, 12 (2017): 1590-1599; Shaw et al., Journal of Clinical Oncology 2018 36:15_suppl, 9008-9008; Besse et al., Journal of Clinical Oncology 2018 36:15_suppl, 9032-9032; and Shaw et al., Cancer Res Jul. 1 2018 (78) (13 Supplement) CT044).

1. A method of identifying an individual having a cancer who may benefit from a treatment comprising an anaplastic lymphoma kinase (ALK)-targeted therapy, the method comprising detecting in a sample from the individual:
(a) an ALK fusion nucleic acid molecule comprising a fusion between an ALK gene, or a portion thereof, and a gene listed in Table 1, or a portion thereof, or
(b) an ALK fusion polypeptide encoded by the ALK fusion nucleic acid molecule,
wherein detection of the ALK fusion nucleic acid molecule or polypeptide in the sample identifies the individual as one who may benefit from the treatment comprising the ALK-targeted therapy.

2. A method of identifying one or more treatment options for an individual having a cancer, the method comprising:
(a) detecting in a sample from the individual:
(i) an ALK fusion nucleic acid molecule comprising a fusion between an ALK gene, or a portion thereof, and a gene listed in Table 1, or a portion thereof, or
(ii) an ALK fusion polypeptide encoded by the ALK fusion nucleic acid molecule; and
(b) generating a report comprising one or more treatment options identified for the individual based at least in part on detection of the ALK fusion nucleic acid molecule or polypeptide in the sample, wherein the one or more treatment options comprise an ALK-targeted therapy.

3. A method of treating or delaying progression of cancer in an individual in need thereof, comprising:
(a) acquiring knowledge of:
(i) an ALK fusion nucleic acid molecule comprising a fusion between an ALK gene, or a portion thereof, and a gene listed in Table 1, or a portion thereof, or
(ii) an ALK fusion polypeptide encoded by the ALK fusion nucleic acid molecule,
in a sample from an individual having a cancer; and
(b) responsive to said knowledge, administering to the individual an effective amount of a treatment that comprises an ALK-targeted therapy.

4. The method of claim 1, wherein:
(a) the ALK fusion nucleic acid molecule is an ALK fusion nucleic acid molecule listed in Table 2, and wherein the order of the genes in the fusion in the 5′ to 3′ direction is as listed in Table 2;
(b) the ALK fusion nucleic acid molecule is an ALK fusion nucleic acid molecule listed in Table 3, and wherein the ALK fusion nucleic acid molecule comprises or results from a corresponding 5′ breakpoint and/or 3′ breakpoint within the exons or introns as listed in Table 3;
(c) the ALK fusion nucleic acid molecule is an ALK fusion nucleic acid molecule listed in Table 4, and wherein the ALK fusion nucleic acid molecule comprises or results from a corresponding 5′ breakpoint within the chromosomal coordinates as listed in Table 4, and/or a corresponding 3′ breakpoint within the chromosomal coordinates as listed in Table 4;
(d) the ALK fusion nucleic acid molecule is an ALK fusion nucleic acid molecule listed in Table 5, and wherein the ALK fusion nucleic acid molecule comprises or results from a fusion of a corresponding 5′ exon as listed in Table 5, or a portion thereof, fused to a corresponding 3′ exon as listed in Table 5, or a portion thereof;
(e) the ALK fusion nucleic acid molecule is an ALK fusion nucleic acid molecule listed in Table 6, and wherein the ALK fusion nucleic acid molecule comprises, in the 5′ to 3′ direction, the corresponding exons or portions thereof as listed in Table 6;
(f) the ALK fusion nucleic acid molecule is an ALK fusion nucleic acid molecule listed in Table 7, and wherein the ALK fusion nucleic acid molecule comprises a corresponding nucleotide sequence as listed in Table 7, or a nucleotide sequence with at least about 70% homology thereto; and/or
(g) the ALK fusion nucleic acid molecule comprises a nucleotide sequence encoding an ALK fusion polypeptide that comprises an amino acid sequence as listed in Table 8, or an amino acid sequence with at least about 70% homology thereto.

5. The method of claim 1, wherein the ALK fusion polypeptide encoded by the ALK fusion nucleic acid molecule:
(a) comprises an ALK kinase domain, or a fragment of an ALK kinase domain having ALK kinase activity;
(b) has ALK kinase activity;
(c) has constitutive ALK kinase activity;
(d) is oncogenic;
(e) promotes cancer cell survival, angiogenesis, cancer cell proliferation, and any combination thereof;
(f) is capable of dimerizing with an ALK polypeptide or with another ALK fusion polypeptide in a cancer cell; and/or
(g) is an ALK fusion polypeptide listed in Table 8, and wherein the ALK fusion polypeptide comprises a corresponding amino acid sequence as listed in Table 8, or an amino acid sequence with at least about 70% homology thereto.

6. The method of claim 1, wherein the cancer is:
(a) a carcinoma, a sarcoma, a lymphoma, a leukemia, a myeloma, a germ cell cancer, or a blastoma;
(b) a solid tumor;
(c) a hematologic malignancy;
(d) a lymphoma;
(e) a non-small cell lung carcinoma, a leiomyosarcoma, a thyroid carcinoma, a colorectal cancer, a pancreatic cancer, or a malignant peritoneal mesothelioma;
(f) a B cell cancer, multiple myeloma, a melanoma, breast cancer, lung cancer, bronchus cancer, colorectal cancer, prostate cancer, pancreatic cancer, stomach cancer, ovarian cancer, urinary bladder cancer, brain cancer, central nervous system cancer, peripheral nervous system cancer, esophageal cancer, cervical cancer, uterine cancer, endometrial cancer, cancer of an oral cavity, cancer of a pharynx, liver cancer, kidney cancer, testicular cancer, biliary tract cancer, small bowel cancer, appendix cancer, salivary gland cancer, thyroid gland cancer, adrenal gland cancer, osteosarcoma, chondrosarcoma, a cancer of hematological tissue, an adenocarcinoma, an inflammatory myofibroblastic tumor, a gastrointestinal stromal tumor (GIST), colon cancer, myelodysplastic syndrome (MDS), myeloproliferative disorder (MPD), acute lymphocytic leukemia (ALL), acute myelocytic leukemia (AML), chronic myelocytic leukemia (CML), chronic lymphocytic leukemia (CLL), polycythemia Vera, Hodgkin lymphoma, non-Hodgkin lymphoma (NHL), soft-tissue sarcoma, fibrosarcoma, myxosarcoma, liposarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, seminoma, embryonal carcinoma, Wilms' tumor, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroblastoma, retinoblastoma, follicular lymphoma, diffuse large B-cell lymphoma, mantle cell lymphoma, hepatocellular carcinoma, thyroid cancer, gastric cancer, head and neck cancer, small cell cancer, essential thrombocythemia, agnogenic myeloid metaplasia, hypereosinophilic syndrome, systemic mastocytosis, familiar hypereosinophilia, chronic eosinophilic leukemia, neuroendocrine cancers, or a carcinoid tumor; or
(g) an anus squamous cell carcinoma, brain glioblastoma (GBM), breast cancer, breast carcinoma, breast invasive ductal carcinoma (IDC), colon adenocarcinoma (CRC), esophagus adenocarcinoma, fallopian tube serous carcinoma, gallbladder adenocarcinoma, gallbladder carcinoma, gastroesophageal junction adenocarcinoma, lung adenocarcinoma, lung non-small cell lung carcinoma, lung non-small cell lung carcinoma, lymph node Castleman's disease, lymph node lymphoma T-cell, ovary clear cell carcinoma, ovary endometrioid adenocarcinoma, ovary epithelial carcinoma, ovary high grade serous carcinoma, ovary serous carcinoma, pancreas cancer, pancreas ductal adenocarcinoma, pediatric bone osteosarcoma, bone osteosarcoma, pediatric skin melanoma, skin melanoma, pediatric soft tissue sarcoma, soft tissue sarcoma, pediatric soft tissue sarcoma undifferentiated, soft tissue sarcoma undifferentiated, peritoneum serous carcinoma, prostate acinar adenocarcinoma, small intestine adenocarcinoma, soft tissue leiomyosarcoma, soft tissue liposarcoma, thyroid papillary carcinoma, unknown primary adenocarcinoma, unknown primary carcinoma, unknown primary malignant neoplasm, unknown primary myoepithelial carcinoma, uterus carcinosarcoma, uterus endometrial adenocarcinoma endometrioid, uterus leiomyosarcoma, or vulva squamous cell carcinoma (SCC).

7. The method of claim 1, wherein the ALK fusion nucleic acid molecule is an ALK fusion nucleic acid molecule listed in Table 9 or the ALK fusion polypeptide is an ALK fusion polypeptide listed in Table 9, and wherein the cancer is the corresponding cancer as listed in Table 9.

8. The method of claim 1, wherein:
(a) the cancer is metastatic;
(b) the cancer has metastasized to the brain of the individual;
(c) the individual has an intracranial metastasis of the cancer or an extracranial metastasis of the cancer;
(d) the cancer has not metastasized to the brain of the individual;
(e) the individual does not have an intracranial metastasis of the cancer; or
(f) the individual does not have an extracranial metastasis of the cancer.

9. The method of claim 1, wherein the ALK-targeted therapy:
(a) comprises one or more of a small molecule inhibitor, an antibody, a cellular therapy, a nucleic acid, a virus-based therapy, an antibody-drug conjugate, a recombinant protein, a fusion protein, a natural compound, a peptide, a PROteolysis-TArgeting Chimera (PROTAC), a treatment for ALK-positive or ALK-rearranged cancer, an ALK-targeted therapy being tested in a clinical trial, a treatment for ALK-positive or ALK-rearranged cancer being tested in a clinical trial, or any combination thereof;
(b) is a kinase inhibitor;
(c) is a tyrosine kinase inhibitor, a kinase inhibitor that inhibits the kinase activity of an ALK polypeptide, a multi-kinase inhibitor, or an ALK-specific inhibitor; or
(d) comprises one or more of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, TAE684 (NVP-TAE684), CT-707, WX-0593, alkotinib, SIM1803-1A, PLB1003, SAF-189s, PF03446962, TQ-B3101, APG-2449, X-376, CEP-28122, and GSK1838705A.

10. The method of claim 1, wherein:
(a) the individual has received a prior anti-cancer treatment, or is being treated with an anti-cancer treatment;
(b) the cancer has not been previously treated;
(c) the cancer has not been previously treated with crizotinib;
(d) the cancer has not been previously treated with a kinase inhibitor;
(e) the cancer has been previously treated with a kinase inhibitor; or
(f) the cancer progressed on a prior treatment with a kinase inhibitor or is refractory to a prior kinase inhibitor treatment.

11. The method of claim 10, wherein the ALK fusion nucleic acid molecule and/or the ALK fusion polypeptide encoded by the ALK fusion nucleic acid molecule confer resistance of the cancer to the prior anti-cancer treatment.

12. The method of claim 1, wherein the cancer further comprises one or more mutations or rearrangements in an ALK kinase domain encoded by an ALK gene, and/or the ALK fusion nucleic acid molecule encodes an ALK fusion polypeptide comprising an ALK kinase domain, or a portion thereof, comprising one or more mutations in the ALK kinase domain.

13. The method of claim 1, wherein the treatment further comprises an additional anti-cancer therapy.

14. The method of claim 13, wherein the additional anti-cancer therapy comprises: (a) one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, a vaccine, a small molecule agonist, a virus-based therapy, an antibody-drug conjugate, a recombinant protein, a fusion protein, a natural compound, a peptide, a PROteolysis-TArgeting Chimera (PROTAC), or any combination thereof; or (b) one or more of a heat shock protein 90 inhibitor, an EGFR inhibitor, a SHP2 inhibitor, a MEK inhibitor, an IGF-1R inhibitor, a vascular endothelial growth factor (VEGF)-targeted therapy, or an mTOR inhibitor.

15. The method of claim 1, wherein:
(a) the method further comprises obtaining the sample from the individual;
(b) the sample is obtained from the cancer;
(c) the sample comprises a tissue biopsy sample, a tumor biopsy sample, a tumor specimen, a liquid biopsy sample, a normal control, circulating tumor cells, circulating tumor DNA (ctDNA), cell-free DNA, or cell-free RNA; and/or
(d) the sample comprises cells and/or nucleic acids from the cancer.

16. The method of claim 3, wherein the acquiring knowledge of the ALK fusion nucleic acid molecule or the ALK fusion polypeptide encoded by the ALK fusion nucleic acid molecule comprises detecting the ALK fusion nucleic acid molecule or polypeptide in the sample.

17. The method of claim 1, wherein: (a) detecting the ALK fusion nucleic acid molecule in the sample comprises detecting a fragment of the ALK fusion nucleic acid molecule comprising a breakpoint or fusion junction between the ALK gene, or the portion thereof, and the gene listed in Table 1, or the portion thereof; and/or (b) detecting the ALK fusion polypeptide comprises detecting a portion of the ALK fusion polypeptide that is encoded by a fragment of the ALK fusion nucleic acid molecule that comprises a breakpoint or fusion junction between the ALK gene, or the portion thereof, and the gene listed in Table 1, or the portion thereof.

18. The method of claim 1, wherein:
(a) the ALK fusion nucleic acid molecule is detected in the sample by one or more of: a nucleic acid hybridization assay, an amplification-based assay, a polymerase chain reaction-restriction fragment length polymorphism (PCR-RFLP) assay, real-time PCR, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), mass-spectrometric genotyping, or sequencing; and/or
(b) the ALK fusion polypeptide is detected in the sample by one or more of:
immunoblotting, enzyme linked immunosorbent assay (ELISA), immunohistochemistry, or mass spectrometry.

19. The method of claim 1, further comprising acquiring knowledge of or detecting in a sample from the individual a base substitution, a short insertion/deletion (indel), a copy number alteration, or a genomic rearrangement in one or more genes.

20. The method of claim 1, wherein the individual is a human.
